(19) United States
(12) Patent Application Publication
(10) Pub. No.: US 2004/0005667 A1
(43) Pub. Date: Jan. 8, 2004

(54) Immunisation against chlamydia pneumoniae

(76) Inventors: Giuloi Ratti, Siena (IT); Guido Grandi, Siena (IT)

Correspondence Address: Rebecca Hale, Chiron Corporation, Intellectual Property Law Department R 3, PO Box 8097, Emeryville, CA, 94662-8097, US

(21) Appl. No.: 10/312,273

(22) PCT Filed: Jul. 3, 2001
(86) PCT No.: PCT/IB01/01445

(30) Foreign Application Priority Data
Jul. 3, 2000 (GB) 0016363.4
Jul. 11, 2000 (GB) 0017047.2
Jul. 21, 2000 (GB) 0017938.8
Aug. 7, 2000 (GB) 0019368.0
Aug. 18, 2000 (GB) 0020440.4
Sep. 14, 2000 (GB) 0022583.9
Nov. 10, 2000 (GB) 0027549.5
Dec. 22, 2000 (GB) 0031706.5

(51) Int. Cl. C07K 14/295; A61K 39/02; C07H 21/04; C12P 21/02; C12N 1/21; C12N 15/74; C12N 15/09; C12N 1/20; C12N 15/00; C12N 15/63; C12N 15/70; C07K 1/00; C07K 14/00; C07K 17/00

(52) U.S. Cl. 435/69.3; 435/320.1; 435/252.3; 530/350; 424/190.1; 536/23.7

(57) ABSTRACT

The published genomic of Chlamydia pneumoniae reveals over 1000 putative encoded proteins but does not itself indicate which of these might to useful antigens for immunisation and vaccination or for diagnosis. This difficulty is addressed by the invention, which provides a number of C. pneumoniae protein sequences suitable for vaccine production and development and/or for diagnostic purposes.

All documents cited herein are incorporated by reference in their entirety.

### TECHNICAL FIELD

This invention is in the field of immunisation against chlamydial infection, in particular against infection by Chlamydia pneumoniae.

### BACKGROUND ART

Chlamydiae are obligate intracellular parasites of eukaryotic cells which are responsible for endemic sexually transmitted infections and various other disease syndromes. They occupy an exclusive eubacterial phylogenic branch, having no close relationship to any other known organisms—they are classified in their own order (Chlamydiales) which contains a single family (Chlamydiaceae) which in turn contains a single genus (Chlamydia). A particular characteristic of the Chlamydiae is their unique life cycle, in which the bacterium alternates between two morphologically distinct forms: an extracellular infective form (elementary bodies, EB) and an intracellular non-infective form (reticulate bodies, RB). The life cycle is completed with the re-organization of RB into EB, which subsequently leave the disrupted host cell ready to infect further cells.

Four chlamydial species are currently; known—C. trachomatis, C. pneunioniae, C. pecorum and C. psittaci [e.g. Raulston (1995) Mol Microbiol 15:607-616; Everett (2000) Vet Microbiol 75:109-126]. C. pneumoniae is closely related to C. trachomatis, as the whole genome comparison of at least two isolates from each species has shown [Kalman et al. (1999) Nature Genetics 21:385-389; Read et al. (2000) Nucleic Acids Res 28:1397-406; Stephens et al. (1998) Science 282:754-759]. Based on surface reaction with patient immune sera, the current view is that only one serotype of C. pneumoniae exists world-wide.

C. pneumoniae is a common cause of human respiratory disease. It was first isolated from the conjunctiva of a child in Taiwan in 1965, and was established as a major respiratory pathogen in 1983. In the USA, C. pneumoniae causes approximately 10% of community-acquired pneumonia and 5% of pharyngitis, bronchitis, and sinusitis.

More recently, the spectrum of C. pneumoniae infections has been extended to include atherosclerosis, coronary heart disease, carotid artery stenosis, myocardial infarction, cerebrovascular disease, aortic aneurysm, claudication, and stroke. The association of C. pneumoniae with atherosclerosis is corroborated by the presence of the organism in atherosclerotic lesions throughout the arterial tree and the near absence of the organism in healthy arterial tissue. C. pneumoniae has also been isolated from coronary and carotid atheromatous plaques. The bacterium has also been associated with other acute and chronic respiratory diseases (e.g. otitis media, chronic obstructive pulmonary disease, pulmonary exacerbation of cystic fibrosis) as a result of sero-epidemiologic observations, case reports, isolation or direct detection of the organism in specimens, and successful response to anti-chlamydial antibiotics. To determine whether chronic infection plays a role in initiation or progression of disease, intervention studies in humans have been initiated, and animal models of C. pneumoniae infection have been developed.

Considerable knowledge of the epidemiology of C. pneumoniae infection has been derived from serologic studies using the C. pneunioniae-specific microimmunofluorescence test. Infection is ubiquitous, and it is estimated that virtually everyone is infected at some point in life, with common re-infection. Antibodies against C. pneumoniae are rare in children under the age of 5, except in developing and tropical countries. Antibody prevalence increases rapidly at ages 5 to 14, reaching 50% at the age of 20, and continuing to increase slowly to ˜80% by age 70.

A current hypothesis is that C. pneumoniae can persist in an asymptomatic low-grade infection in very large sections of the human population. When this condition occurs, it believed that the presence of C. pneumoniae, and/or the effects of the host reaction to the bacterium, can cause or help progress of cardiovascular illness.

It is not yet clear whether C. pneumoniae is actually a causative agent of cardiovascular disease, or whether it is just artefactually associated with it. It has been shown, however, that C. pneumoniae infection can induce LDL oxidation by human monocytes [Kalayoglu et al. (1999) J. Infect. Dis. 180:780-90; Kalayoglu et al. (1999) Am. Heart J. 138:S488-490]. As LDL oxidation products are highly atherogenic, this observation provides a possible mechanism whereby C. pneumoniae may cause atheromatous degeneration. If a causative effect is confirmed, vaccination (prophylactic and therapeutic) will be universally recommended.

Genomic sequence information has been published for C. pneumoniae [Kalman et al. (1999) supra; Read et al. (2000) supra; Shirai et al. (2000) J. Infect. Dis. 181 (Suppl 3):S524-S527; WO99/27105; WO00/27994] and is available from GenBank. Sequencing efforts have not, however, focused on vaccination, and the availability of genomic sequence does not in itself indicate which of the >1000 genes might encode useful antigens for immunisation and vaccination. WO99/27105, for instance, implies that every one of the 1296 ORFs identified in the C. pneumoniae strain CM1 genome is a useful vaccine antigen.

It is thus an object of the present invention to identify antigens useful for vaccine production and development from amongst the many proteins present in C. pneumoniae. It is a further object to identify antigens useful for diagnosis (e.g. immunodiagnosis) of C. pneumoniae.

### DISCLOSURE OF THE INVENTION

The invention provides proteins comprising the C. pneumoniae amino acid sequences disclosed in the examples.

It also provides proteins comprising sequences which share at least x% sequence identity with the C. pneumoniae amino acid sequences disclosed in the examples. Depending on the particular sequence, x is preferably 50% or more (e.g. 60%, 70%, 80%, 90%, 95%, 99% or more). These include mutants and allelic variants. Typically, 50% identity or more between two proteins is considered to be an indication of functional equivalence. Identity between proteins is preferably determined by the Smith-Waterman homology search algorithm as implemented in the MPSRCH program (Oxford Molecular), using an affine gap search with parameters gap open penalty=12 and gap extension penalty=1.

The invention further provides proteins comprising fragments of the C. pneumoniae amino acid sequences disclosed in the examples. The fragments should comprise at least n consecutive amino acids from the sequences and, depending on the particular sequence, n is 7 or more (e.g. 8, 10, 12, 14, 16, 18, 20, 30, 40, 50, 75, 100 or more). Preferably the fragments comprise one or more epitope(s) from the sequence. Other preferred fragments omit a signal peptide.

The proteins of the invention can, of course, be prepared by various means (e.g. native expression, recombinant expression, purification from cell culture, chemical synthesis etc.) and in various forms (e.g. native, fusions etc.). They are preferably prepared in substantially pure form (i.e. substantially free from other C. pneumoniae or host cell proteins). Heterologous expression in E. coli is a preferred preparative route.

According to a further aspect, the invention provides nucleic acid comprising the C. pneumoniae nucleotide sequences disclosed in the examples. In addition, the invention provides nucleic acid comprising sequences which share at least x% sequence identity with the C. pneumoniae nucleotide sequences disclosed in the examples. Depending on the particular sequence, x is preferably 50% or more (e.g. 60%, 70%, 80%, 90%, 95%, 99% or more).

Furthermore, the invention provides nucleic acid which can hybridise to the C. pneumoniae nucleic acid disclosed in the examples, preferably under “high stringency” conditions (e.g. 65° C. in a 0.1×SSC, 0.5% SDS solution).

Nucleic acid comprising fragments of these sequences are also provided. These should comprise at least n consecutive nucleotides from the C. pneumoniae sequences and, depending on the particular sequence, n is 10 or more (e.g. 12, 14, 15, 18, 20, 25, 30, 35, 40, 50, 75, 100, 200, 300 or more).

According to a further aspect, the invention provides nucleic acid encoding the proteins and protein fragments of the invention.

It should also be appreciated that the invention provides nucleic acid comprising sequences complementary to those described above (e.g. for antisense or probing purposes).

Nucleic acid according to the invention can, of course, be prepared in many ways (e.g. by chemical synthesis, from genomic or cDNA libraries, from the organism itself etc.) and can take various forms (e.g. single stranded, double stranded, vectors, probes etc.).

In addition, the term “nucleic acid” includes DNA and RNA, and also their analogues, such as those containing modified backbones, and also peptide nucleic acids (PNA) etc.

According to a further aspect, the invention provides vectors comprising nucleotide sequences of the invention (e.g. cloning or expression vectors) and host cells transformed therewith.

According to a further aspect, the invention provides immunogenic compositions comprising protein and/or nucleic acid according to the invention. These compositions are suitable for immunisation and vaccination purposes. Vaccines of the invention may be prophylactic or therapeutic, and will typically comprise an antigen which can induce antibodies capable of inhibiting (a) chlamydial adhesion, (b) chlamydial entry, and/or (c) successful replication within the host cell. The vaccines preferably induce any cell-mediated T-cell responses which are necessary for chlamydial clearance from the host.

The invention also provides nucleic acid or protein according to the invention for use as medicaments (e.g. as vaccines). It also provides the use of nucleic acid or protein according to the invention in the manufacture of a medicament (e.g. a vaccine or an immunogenic composition) for treating or preventing infection due to C. pneumoniae.

The invention also provides a method of treating (e.g. immunising) a patient, comprising administering to the patient a therapeutically effective amount of nucleic acid or protein according to the invention.

According to further aspects, the invention provides various processes.

A process for producing proteins of the invention is provided, comprising the step of culturing a host cell according to the invention under conditions which induce protein expression.

A process for producing protein or nucleic acid of the invention is provided, wherein the protein or nucleic acid is synthesised in part or in whole using chemical means.

A process for detecting C. pneumoniae in a sample is provided, wherein the sample is contacted with an antibody which binds to a protein of the invention.

A summary of standard techniques and procedures which may be employed in order to perform the invention (e.g. to utilise the disclosed sequences for immunisation) follows. This summary is not a limitation on the invention but, rather, gives examples that may be used, but are not required.

General

The practice of the present invention will employ, unless otherwise indicated, conventional techniques of molecular biology, microbiology, recombinant DNA, and immunology, which are within the skill of the art. Such techniques are explained fully in the literature e.g. Sambrook Molecular Cloning; A Laboratory Manual, Second Edition (1989) and Third Edition (2001); DNA Cloning, Volumes I and ii (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

Definitions

A composition containing X is “substantially free of” Y when at least 85% by weight of the total X+Y in the composition is X. Preferably, X comprises at least about 90% by weight of the total of X+Y in the composition, more preferably at least about 95% or even 99% by weight,

The term “comprising” means “including” as well as “consisting” e.g. a composition “comprising” X may consist exclusively of X or may include something additional to X, such as X+Y.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a Chlamydial sequence is heterologous to a mouse host cell. A further examples would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (e.g. 60%, 70%, 80%, 90%, 95%, 99% or more, calculated using the Smith-Waterman algorithm as described above). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions (e.g. see U.S. Pat. No. 5,753,235).

Expression Systems

The Chlamydial nucleotide sequences can be expressed in a variety of different expression systems; for example those used with mammalian cells, baculoviruses, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (e.g. structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation [Sambrook et al. (1989) “Expression of Cloned Genes in Mammalian Cells.” In Molecular Cloning: A Laboratory Manual, 2nd ed.].

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter [Maniatis et al. (1987) Science 236:1237; Alberts et al. (1989) Molecular Biology of the Cell, 2nd ed.]. Enhancer elements derived from viruses may be particularly useful, because they usually have a broader host range. Examples include the SV40 early gene enhancer [Dijkema et al (1985) EMBO J. 4:761] and the enhancer/promoters derived from the long terminal repeat (LTR) of the Rous Sarcoma Virus [Gorman et al. (1982) PNAS USA 79:6777] and from human cytomegalovirus [Boshart et al. (1985) Cell 41:521.] Additionally, some enhancers are regulatable and become active only in the presence of an inducer, such as a hormone or metal ion [Sassone-Corsi and Borelli (1986) Trends Genet. 2:215; Maniatis et al. (1987) Science 236:1237].

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation [Birnstiel et al. (1985) Cell 41:349; Proudfoot and Whitelaw (1988) “Termination and 3′ end processing of eukaryotic RNA. In Transcription and splicing (ed. B. D. Hames and D. M. Glover); Proudfoot (1989) Trends Biochem. Sci. 14:105]. These sequences direct the transcription of an mRNA which can be translated into the polypeptide encoded by the DNA. Examples of transcription terminater/polyadenylation signals include those derived from SV40 [Sambrook et al (1989) “Expression of cloned genes in cultured mammalian cells.” In Molecular Cloning: A Laboratory Manual].

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g. plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 [Gluzman (1981) Cell 23:175] or polyomavirus, replicate to extremely high copy number in the presence of the appropriate viral T antigen. Additional examples of mammalian replicons include those derived from bovine papillomavirus and Epstein-Barr virus. Additionally, the replicon may have two replicaton systems, thus allowing it to be maintained, for example, in mammalian cells for expression and in a prokaryotic host for cloning and amplification. Examples of such mammalian-bacteria shuttle vectors include pMT2 [Kaufman et al. (1989) Mol. Cell. Biol. 9:946] and pHEBO [Shimizu et al. (1986) Mol. Cell. Biol. 6:1074].

The transformation procedure used depends upon the host to be transformed. Methods for introduction of heterologous polynucleotides into mammalian cells are known in the art and include dextran-mediated transfection, calcium phosphate precipitation, polybrene-mediated transfection, protoplast fusion, electroporation, encapsulation of polynucleotide(s) in liposomes, direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g. Hep G2), and a number of other cell lines.

ii. Baculovirus Systems

The polynucleotide encoding the protein can also be inserted into a suitable insect expression vector, and is, operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media,

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego, Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence of interest, and transcription termination sequence, are usually assembled into an intermediate transplacement construct (transfer vector). This construct may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extrachromosomal element (e.g. plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATO to ATT, and which introduces a BamHI cloning site 32 basepairs downstream from the ATT; see Luckow and Summers, Virology (1989)17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol. 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon, Maeda et al., (1985), Nature 315:592; human gastrin-releasing peptide, Lebacq-Verheyden et al., (1988), Molec. Cell. Biol. 8:3129; human IL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect-cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91.The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between ˜1% and ˜5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skilled in the art. Namely, the plaques are screened under the light microscope for the presence (indicative of wild-type virus) or absence (indicative of recombinant virus). of occlusion bodies. “Current Protocols in Microbiology” Vol. 2 (Ausubel et al. eds) at 16.8 (Supp. 10, 1990); Summers & Smith, supra; Miller et al. (1989).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, e.g. Summers and Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g. HPLC, affinity chromatography, ion exchange chromatography, etc.; electrophoresis; density gradient centrifugation; solvent extraction, or the like. As appropriate, the product may be further purified, as required, so as to remove substantially any insect proteins which are also secreted in the medium or result from lysis of insect cells, so as to provide a product which is at least substantially free of host debris, e.g. proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iii. Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology,. Malcolm B. Wilkins, ed., 1984 Pitman Publishing Limited, London, pp. 21-52. References that describe other metabolically-regulated genes: Sheen, Plant Cell, 2:1027-1038(1990); Maas et al., EMBO J. 9:3447-3452 (1990); Benkel and Hickey, Proc. Natl. Acad. Sci. 84:1337-1339 (1987)

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11(2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome,. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension, These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacterial Systems

Bacterial expression techniques are known in the art. A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al. (1984) Annu. Rev. Genet. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (trp) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The g-laotamase (bla) promoter system [Weissmann (1981) “The cloning of interferon and other mistakes.” In Interferon 3 (ed. I. Gresser)], bacteriophage lambda PL [Shimatake et al. (1981) Nature 292:128] and T5 [U.S. Pat. No. 4,689,406] promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid trp-lac promoter comprised of both trp promoter and lac operon sequences that is regulated by the lac repressor [Amann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EPO-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of (he initiation codon [Shine et al. (1975) Nature 254:34]. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA.” In Biological Regulation and Development: Gene Expression (ed, R. F. Goldberger)]. To express eukaryotic genes and prokaryotic genes with weak ribosome-binding site [Sambrook et al. (1989) “Expression of cloned genes in Escherichia coli.” In Molecular Cloning: A Laboratory Manual].

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EPO-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene [Nagai et al. (1984) Nature 309:810]. Fusion proteins can also be made with sequences from the lacZ [Jia et al. (1987) Gene 60:1971, trpE [Allen et al. (1987) J. Biotechnol. 5:93; Makoff et al. (1989) J. Gen. Microbiol. 135:11], and Chey [EP-A-0 324 647] genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g. ubiquitin specific processing-protease) to cleave the ubiquitin from the foreign protein. Through this method, native foreign protein can be isolated [Miller et al. (1989) Bio/Technology 7:698].

Alternatively, foreign proteins can also be secreted from the cell by creating chimeric DNA molecules that encode a fusion protein comprised of a signal peptide sequence fragment that provides for secretion of the foreign protein in bacteria [U.S. Pat. No. 4,336,336]. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb et al. (1984) EMBO J. 3:2437] and the E. coli alkaline phosphatase signal sequence (phoA) [Oka et al. (1985) Proc. Natl. Acad. Sci. 82:7212). As an additional example, the signal sequence of the alpha-amylase gene from various Bacillus strains can be used to secrete heterologous proteins from B. subtilis [Palva et al. (1982) Proc. Natl. Acad. Sci. USA 79:5582; EP-A-0 244 042].

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EP-A- 0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Annu. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis [Palva et al. (1982) Proc. Natl. Acad. Sci. USA 79:5582; EP-A-0 036 259 and EP-A-0 063 953; WO 84/04541], Escherichia coli [Shimatake et al. (1981) Nature 292:128; Amann et al. (1985) Gene 40:183; Studier et al. (1986) J. Mol. Biol. 189:113; EP-A-0 036 776,EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. See e.g. [Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus], [Miller et al. (1988) Proc. Natl. Acad. Sci. 85:856; Wang et al. (1990) J. Bacteriol. 172:949, Campylobacter], [Cohen et al. (1973) Proc. Natl. Acad. Sci. 69:2110; Dower et al. (1988) Nucleic Acids Res. 16:6127;.Kushner (1978) “An improved method for transformation of Escherichia coli with ColE1-derived plasmids. In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus].

v. Yeast Expression

Yeast expression systems are also known to one of ordinary skill in the art. A yeast promoter is any DNA sequence capable of binding yeast RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PHO5 gene, encoding acid phosphatase, also provides useful promoter sequences [Myanohara et al. (1983) Proc. Natl. Acad. Sci. USA 80:1].

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (BPA-0 164 556). Furthermore, a yeast promoter can include naturally occurring promoters of non-yeast origin that have the ability to bind yeast RNA polymerase and initiate transcription. Examples of such promoters include, inter alia, [Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109;].

A DNA molecule may be expressed intracellularly in yeast, A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See e.g. EP-A-0 196 056. Another example is a ubiquitin fusion protein. Such a fusion protein is made with the ubiquitin region that preferably retains a site for a processing enzyme (e.g. ubiquitin-specific processing protease) to cleave the ubiquitin from the foreign protein. Through this method, therefore, native foreign protein can be isolated (e.g. WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the genes for invertase (EP-A-0012873; JPO 62,096,086) and A-factor (U.S. Pat. No. 4,588,684). Alternatively, leaders of non-yeast origin exit, such as an interferon leader, that also provide for secretion in yeast (EP-A-0060057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alphafactor. (e.g. see WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (e.g. plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 (Botstein et al. (1979) Gene 8:17-24], pC1/1 [Brake et al. (1984) Proc. Natl. Acad. Sci USA 81:4642-4646], and YRp17 [Stinchcomb et al. (1982) J. Mol. Biol. 158:157]. In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See e.g. Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome [Orr-Weaver et al. (1983) Methods in Enzymol. 101:228-245]. An integrating vector may be directed to a specific locus in yeast by selecting the appropriate homologous sequence for inclusion in the vector. See Orr-Weaver et al., supra. One or more expression construct may integrate, possibly affecting levels of recombinant protein produced [Rine et al. (1983) Proc. Natl. Acad. Sci. USA 80:6750]. The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions [Butt et al. (1987) Microbiol. Rev. 51:351].

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts: Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis [Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol. 25:141], Pichia pastoris [Cregg, et al. (1985) Mol. Cell. Biol. 5:3376; U.S. Pat. Nos. 4,837,148 and 4,929,555], Saccharomyces cerevisiae [Hinnen et al. (1978) Proc. Natl. Acad. Sci. USA 75:1929; Ito et al. (1983) J. Bacteriol. 153:163], Schizosaccharomyces pombe [Beach and Nurse (1981) Nature 300:706], and Yarrowia lipolytica [Davidow, et al. (1985) Curr. Genet. 10:380471 Gaillardin, et al. (1985) Curr. Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed, See e.g. [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al, (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 & 4,929,555; Pichia]; [Hinnen et al. (1978) Proc. Natl. Acad. Sci. USA 75;1929; Ito et al. (1983) J. Bacteriol, 153:163 Saccharomyces]; [Beach & Nurse (1981) Nature 300:706; Schizosaccharomyces]; [Davidow et al. (1985) Curr. Genet. 10:39; Gaillardin et al. (1985) Curr. Gentet. 10:49; Yarrowia].

Pharmaceutical Compositions

Pharmaceutical compositions can comprise polypeptides and/or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For purposes of the present invention, an effective dose will be from about 0.01 mg/ kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol, Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

Vaccines

Vaccines according to the invention may either be prophylactic (i.e. to prevent infection) or therapeutic (i.e. to treat disease after infection).

Such vaccines comprise immunising antigen(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59™ (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (e.g. IL-1, IL-2, IL-4, IL-5, IL-6, IL-7, IL-12, etc.), interferons (e.g. gamma interferon), macrophage colony stimulating factor (M-CSP), tumor necrosis factor (TNP), etc; and (6) other substances that act as immunostimulating agents to enhance the effectiveness of the composition. Alum and MF59™ are preferred.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (MTP-PE), etc.

The immunogenic compositions (e.g. the immunising antigen/immunogen/polypeptide/protein/ nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g. nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, e.g. by injection, either subcutaneously, intramuscularly, or transdermally/transcutaneously (e.g. WO98/20734). Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

As an alternative to protein-based vaccines, DNA vaccination may be employed [e.g. Robinson & Torres (1997) Seminars in Immunology 9:271-283; Donnelly et al. (1997) Annu Rev Immunol 15:617-648; see later herein].

Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153.

Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see O'Neill (1985) J. Virol. 53:160) polytropic retroviruses e.g. MCF and MCF-MLV (see Kelly (1983) J. Virol. 45:291), spumaviruses and lentiviruses. See RNA Tumor Viruses, Second Edition, Cold Spring Harbor Laboratory, 1985.

Portions of the retroviral gene therapy vector may be derived from different retroviruses, For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (e.g. HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J Virol 19:19-25), Abelson (ATCC No. VR-999), Friend (ATCC No. VR-245), Graffi, Gross (ATCC Nol VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936, WO94/03622, WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-967; Ram (1993) Cancer Res 53 (1993) 83-88; Takamiya (1992) J Neurosci Res 33:493-503; Baba (1993) J Neurosurg 79:729-735; Mann (1983) Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/11984, WO95/00655, WO95/27071, WO95/29993, WO95/34671, WO96/05320, WO94/08026, WO94/11506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curiel (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (i.e. there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector. Construction of the Double-D ITR vector is disclosed in U.S. Pat. No. 5,478,745. Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar), pHSVlac described in Geller (1988) Science 241:1667-1669 and in WO90/09441 & WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with ATCC as accession numbers ATCC VR-977 and ATCC VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995,WO94/21792, WO92/10578, WO95/07994, U.S. Pat. No. 5,091,309 and U.S. Pat. No. 5,217,879 arc employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679640).

DNA vector systems such as eukaryotic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1110 and those described in Arnold (1990) J Cell Biochent L401;,pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243, Kyzylagach virus, for example ATCC VR-927; Mayaro virus, for example ATCC VR-66; Mucambo virus, for example ATCC VR-580 and ATCC VR-1244; Ndumu virus, for example ATCC VR-371; Pixuna virus, for example ATCC VR-372 and ATCC VR-1245; Tonate virus, for example ATCC VR-925; Triniti virus, for example ATCC VR-469, Una virus, for example ATCC VR-374; Whataroa virus, for example ATCC VR-926; Y-62-33 virus, for example ATCC VR-375; O'Nyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curiel (1992) Hum Gene Ther 3:147-154 ligand linked DNA, for example see Wu (1989) J Biol Chem 264:16985-16987, eucaryotic cell delivery vehicles cells, for example see U.S. Ser. No.08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/11033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Exemplary naked DNA introduction methods are described in WO90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then he incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033

Exemplary liposome and polycationic gene delivery vehicles are those described in U.S. Pat. No. 5,422,120 and 4,762,915; in WO 95/13796; WO94/23697; and WO91/14445; in EP-0524968; and in Stryer, Biochemistry, pages 236-240 (1975) W. H. Freeman, San Francisco; Szoka (1980) Biochem Biophys Acta 600:1; Bayer (1979) Biochem Biophys Acta 550:464; Rivnay (1987) Meth Enzymol 149:119; Wang (1987) Proc Natl Acad Sci 84:7851; Plant (1989) Anal Biochem 176:420.

A polynucleotide composition can comprises therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

Delivery Methods

Once formulated, the polynucleotide compositions of the invention can be administered (I) directly to the subject; (2) delivered ex vivo, to cells derived from the subject; or (3) in vitro for recombinant protein expression. The subjects to be treated can be mammals or birds. Also, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

Methods for the ex vivo delivery and reimplantation of transformed cells into a subject are known in the art and described in e.g. WO93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Polynucleotide and Polypeptide Pharmaceutical Compositions

In addition to the pharmaceutically acceptable carriers and salts described above, the following additional agents can be used with polynucleotide and/or polypeptide compositions.

A. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

B. Hormones, Vitamins, etc.

Other groups that can be included are, for example: hormones, steroids, androgens, estrogens, thyroid hormone, or vitamins, folic acid.

C. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

D. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Feigner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N[1,2,3-dioleyloxy)propyl]-N,N,N-triethylammonium (DOTMA) liposomes are available under the trademark Lipofectin, from GIBCO BRL, Grand Island, N.Y. (See, also, Feigner supra). Other commercially available liposomes include transfectace (DDAB/DOPE) and DOTAP/DOPE (Boerhinger). Other cationic liposomes can be prepared from readily available materials using techniques well known in the art. See, e.g. Szoka (1978) Proc. Natl. Acad. Sci, USA 75:4194-4198; WO90/11092 for a description of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios, Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See e.g. Straubinger (1983) Meth. Immuol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145;Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

E. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein, For example, naturally occurring chylomicrons comprises of A, B, C, & E, over time these lipoproteins lose A and acquire C and E apoproteins. VLDL comprises A, B, C, & E apoproteins, LDL comprises apoprotein B; HDL comprises apoproteins A, C, & E.

The amino acid of these apoproteins are known and are described in, for example, Breslow (1985) Annu Rev. Biochem 54:699; Law (1986) Adv. Exp Med. Biol, 151:162; Chen (1986) J Biol Chem 261:12918; Kane (1980) Proc Natl Acad Sci USA 77:2465; and Utermann (1984) Hum Genet 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443. Lipoproteins can also be purchased from commercial suppliers, such as Biomedical Techniologies, Inc., Stoughton, Mass., USA. Further description of lipoproteins can be found in Zuckermann et al. PCT/US97/14465.

F. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (X174, transcriptional factors also contain domains that bind DNA and therefore may be useful as nucleic aid condensing agents. Briefly, transcriptional factors such as C/CEBP, c-jun, c-fos, AP-1, AP-2, AP-3, CPP, Prot-1, Sp-1, Oct-1, Oct-2, CREP, and TFIID contain basic domains that bind DNA sequences.

Organic polycationic agents include: spermine, spermidine, and purtrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene, Lipofectin™, and lipofectAMINE™ are monomers that form polycationic complexes when combined with polynocleotides/polypeptides.

Nucleic Acid Hybridisation

“Hybridization” refers to the association of two nucleic acid sequences to one another by hydrogen bonding, Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. [supra] vol.2, chapt.9, pp.9.47 to 9.57.

“Stringency” refers to conditions in a hybridization reaction that favor association of very similar sequences over sequences that differ. For example, the combination of temperature and salt concentration should be chosen that is approximately 120 to 200° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:

Tm=81+16.6(log₁₀Ci)+0.4[%(G+C)]−0.6(% formamide)−600/n −1.5 (% mismatch).

where Ci is the salt concentration (monovalent ions) and n is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (i.e. stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double stranded complex, which is stable enough to be detected.

The nucleic acid probes will hybridize to the Chlamydial nucleotide sequences of the invention (including both sense and antisense strands). Though many different nucleotide sequences will encode the amino acid sequence, the native Chlamydial sequence is preferred because it is the actual sequence present in cells. mRNA represents a coding sequence and so a probe should be complementary to the coding sequence; single-stranded cDNA is complementary to mRNA, and so a cDNA probe should be complementary to the non-coding sequence.

The probe sequence need not be identical to the Chlamydial sequence (or its complement)—some variation in the sequence and length can lead to increased assay sensitivity if the nucleic acid probe can form a duplex with target nucleotides, which can be detected. Also, the nucleic acid probe can include additional nucleotides to stabilize the formed duplex. Additional Chlamydial sequence may also be helpful as a label to detect the formed duplex. For example, a non-complementary nucleotide sequence may be attached to the 5′ end of the probe, with the remainder of the probe sequence being complementary to a Chlamydial sequence. Alternatively, non-complementary bases or longer sequences can be interspersed into the probe, provided that the probe sequence has sufficient complementarity with the a Chlamydial sequence in order to hybridize therewith and thereby form a duplex which can be detected.

The exact length and sequence of the probe will depend on the hybridization conditions, such as temperature, salt condition and the like. For example, for diagnostic applications, depending on the complexity of the analyte sequence, the nucleic acid probe typically contains at least 10-20 nucleotides, preferably 15-25, and more preferably ≧30 nucleotides, although it may be shorter than this. Short primers generally require cooler temperatures to form sufficiently stable hybrid complexes with the template.

Probes may be produced by synthetic procedures, such as the triester method of Matteucci et al. [J. Am. Chem. Soc. (1981) 103:3185], or according to Urdea et al. [Proc. Natl. Acad. Sci. USA (1983) 80: 7461], or using commercially available automated oligonucleotide synthesizers.

The chemical nature of the probe can be selected according to preference. For certain applications, DNA or RNA are appropriate. For other applications, modifications may be incorporated e.g. backbone modifications, such as phosphorothioates or methylphosphonates, can be used to increase in viva half-life, alter RNA affinity, increase nuclease resistance etc. [e.g. see Agrawal & Iyer (1995) Curr Opin Biotechnol 6:12-19; Agrawal (1996) TIBTECH 14:376-387]; analogues such as peptide nucleic acids may also be used (e.g. see Corey (1997) TIBTECH 15:224-229; Buchardt et al. (1993) TIBTECH 11:384-386].

Alternatively, the polymerase chain reaction (PCR) is another well-known means for detecting small amounts of target nucleic acids. The assay is described in: Mullis et al. [Meth. Enzymzol. (1987) 155: 335-350]; U.S. Pat. Nos. 4,683,195 & 4,683,202. Two ‘primers’ hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired Chlamydial sequence.

A thermostable polymerase creates copies of target nucleic acids from the primers using the original target nucleic acids as a template. After a threshold amount of target nucleic acids are generated by the polymerase, they can be detected by more traditional methods, such as Southern blots. When using the Southern blot method, the labelled probe will hybridize to the Chlamydial sequence (or its complement).

Also, mRNA or cDNA can be detected by traditional blotting techniques described in Sambrook et al [supra]. mRNA, or cDNA generated from mRNA using a polymerase enzyme, can be purified and separated using gel electrophoresis. The nucleic acids on the gel are then blotted onto a solid support, such as nitrocellulose. The solid support is exposed to a labelled probe and then washed to remove any unhybridized probe. Next, the duplexes containing the labeled probe are detected. Typically, the probe is labelled with a radioactive moiety.

### BRIEF DESCRIPTION OF THE DRAWINGS

FIGS. 1-189 show data pertaining to examples 1-189.

FIG. 190 shows a representative 2D gel of proteins in elementary bodies.

FIG. 191 shows an alignment of sequences in five (six) proteins of the invention.

### EXAMPLES

The examples indicate C. pneumoniae proteins, together with evidence to support the view that the proteins are useful antigens for vaccine production and development or for diagnostic purposes. This evidence takes the form of:

Computer prediction based on sequence information from CWL029 strain (e.g. using the PSORT algorithm available from www.psort.nibb.ac.jp).

Data on recombinant expression and purification of the proteins cloned from IOL207 strain.

Western blots to demonstrate immunoreactivity in serum (typically a blot of an EB extract of C. pneumoniae strain FB/96 stained with mouse antiserum against the recombinant protein).

FACS analysis of C. pneumoniae bacteria or purified EBs to confirm accessibility of the antigen to the immune system (see also table III).

An indication if the protein was identified by MALDI-TOF from a 2D gel electrophoresis map of proteins from purified elementary bodies from strain FB/96. This confirms that the protein is expressed in vivo (see also table V).

Various tests can be used to assess the in vivo immunogenicity of the proteins identified in the examples. For example, the proteins can be expressed recombinantly and used to screen patient sera by immunoblot. A positive reaction between the protein and patient serum indicates that the patient has previously mounted an immune response to the protein in question i.e. the protein is an immunogen. This method can also be used to identify immunodominant proteins.

The recombinant protein can also be conveniently used to prepare antibodies e.g. in a mouse. These can be used for direct confirmation that a protein is located on the cell-surface. Labelled antibody (e.g. fluorescent labelling for FACS) can be incubated with intact bacteria and the presence of label on the bacterial surface confirms the location of the protein.

In particular, the following methods (A) to (O) were used to express, purify and biochemically characterise the proteins of the invention:

### Cloning of CPN ORFs for Expression in E. coli

ORFs of Chlamydia pneumoniae (Cpn) were cloned in such a way as to potentially obtain three different kind of proteins:

a) proteins having an hexa-histidine tag at the C-terminus (cpn-His)

b) proteins having a GST fusion partner at the N-terminus (Gst-cpn)

c) proteins having both hexa-histidine tag at the C-terminus and GST at the N-terminus (GST/His fusion; NH₂-GST-cpn-(His)₆-COOH)

The type a) proteins were obtained upon cloning in the pET21b+ (Novagen). The type b) and c) proteins were obtained upon cloning in modified pGEX-KG vectors [Guan & Dixon (1991) Anal. Biochem. 192:262]. For instance pGEX-KG was modified to obtain pGEX-NN, then by modifying pGEX-NN to obtain pGEX-NNH. The Gst-cpn and Gst-cpn-His proteins were obtained in pGEX-NN and pGEX-NNH respectively.

The modified versions of pGEX-KG vector were made with the aim of allowing the cloning of single amplification products in all three vectors after only one double restriction enzyme digestion and to minimise the presence of extraneous amino acids in the final recombinant proteins.

(A) Construction of pGEX-NN and pGEX-NNH Expression Vectors

Two couples of complementary oligodeoxyribonucleotides were synthesised using the DNA synthesiser ABI394 (Perkin Elmer) and the reagents from Cruachem (Glasgow, Scotland). Equimolar amounts of the oligo pairs (50 ng each oligo) were annealed in T4 DNA ligase buffer (New England Biolabs) for 10 min in a final volume of 50 μl and then were left to cool slowly at room temperature. With the described procedure he following DNA linkers were obtained:

```
gexNN linker:
NdeI NheI XmaI EcoRI NcoI SalI XhoI SacI NotI
GATCCCATATGGCTAGCCCGGGGAATTCGTCCATGGAGTGAGTCGACTGACTCGAGTGATCGAGCTCCTGAGCGGCCGCATGAA
        GGTATACCGATCGGGCCCCTTAAGCAGGTACCTCACTCAGCTGACTGAGCTCACTAGCTCGAGGACTCGCCGGCGTACTTTCGA
gexNNH linker:
                      HindIII NotI XhoI --Hexa-Histidine--
            TCGACAAGCTTGCGGCCGCACTCGAGCATCACCATCACCATCACTGAT
                GTTCGAACGCCGGCGTGAGCACGTAGAGGTAGTGGTAGTGACTATCGA
```

The plasmid pGEX-KG was digested with BamHI and HindIII and 100 ng were ligated overnight at 16° C. to the linker gexNN with a molar ratio of 3:1 linker/plasmid using 200 units of T4 DNA ligase (New england Biolabs). After transformation of the ligation product in E. coli DH5, a clone containing the pGEX-NN plasmid, having the correct linker, was selected by means of restriction enzyme analysis and DNA sequencing.

The new plasmid pGEX-NN was digested with SalI and HindIII and ligated to the linker gex-NNH. After transformation of the ligation product in E. coli DH5, a clone containing the pGEX-NNH plasmid, having the correct linker, was selected by means of restriction enzyme analysis and DNA sequencing.

(B) Chromosomal DNA Preparation

The chromosomal DNA of elementary bodies (EB) of C. pneumoniae strain IOL-207 was prepared by adding 1.5 ml of lysis buffer (10 mM Tris-HCl, 150 mM NaCl, 2 mM EDTA, 0,6 % SDS, 100 μg/ml Proteinase K, pH 8) to 450 μl EB suspension (400.000/μl) and incubating overnight at 37° C. After sequential extraction with phenol, phenol-chloroform, and chloroform, the DNA was precipitated with 0.3 M sodium acetate, pH 5.2 and 2 volumes of absolute ethanol. The DNA pellet was washed with 70 % ethanol. After solubilization with distilled water and treatment with 20 μg/ml RNAse A for 1 hour at RT, the DNA was extracted again with phenol-chloroform, alcohol precipitated and suspended with 300 μl 1 mM Tris-HCl pH 8.5. The DNA concentration was evaluated by measuring OD₂₆₀ of the sample.

(C) Oligonucleotide Design

Synthetic oligonucleotide primers were designed on the basis of the coding sequence of each ORF using the sequence of C. pneumoniae strain CWL029. Any predicted signal peptide were omitted, by deducing the 5′ end amplification primer sequence immediately downstream from the predicted leader sequence. For most ORFs, the 5′ tail of the primers (table I) included only one restriction enzyme recognition site (NdeI, or NheI, or SpeI depending on the gene's own restriction pattern); the 3′ primer tails (tablet) included a XhoI or a NotI or a HindIII restriction site.

TABLE I

Oligonucleotide tails of the primers
used to amplify Cpn genes.

| | |
|---|---|
| 5′ tails | 3′ tails |

| | | | |
|---|---|---|---|
| NdeI | 5′ GTGCGTCATATG 3′ | XhoI | 5′ GCGTCTCGAG 3′ |
| NheI | 5′ GTGCGTGCTAGC 3′ | NotI | 5′ ACTCGCTAGCGGCCGC |

| | | |
|---|---|---|
| SpeI | 5′ GTGCGTACTAGT 3′ | 3′ |

| | |
|---|---|
| Hind III | 5′ GCGTAAGCTT 3′ |

As well as containing the restriction enzyme recognition sequences, the primers included nucleotides which hybridized to the sequence to be amplified. The number of hybridizing nucleotides depended on the melting temperature of the primers which was determined as described [(Breslauer et al. (1986) PNAS USA 83:3746-50]. The average melting temperature of the selected oligos was 50-55° C. for the hybridizing region alone and 65-75° C. for the whole oligos. Table II shows the forward and reverse primers used for each amplification.

(D) Amplification

The standard PCR protocol was as follow: 50 ng genomic DNA were used as template in the presence of 0.2 μM each primer, 200 μM each dNTP, 1.5 mM MgCl₂, 1× PCR buffer minus Mg (Gibco-BRL), and 2 units of Taq DNA polymerase (Platinum Taq, Gibco-BRL) in a final volume of 100 μl. Each sample underwent a double-step amplification: the first 5 cycles were performed using as the hybridizing temperature the one of the oligos excluding the restriction enzyme tail, followed by 25 cycles performed according to the hybridization temperature of the whole lenght primers. The standard cycles were as follow:

| denaturation: 94° C., 2 min | | |
|---|---|---|
| denaturation: 94° C., 30 seconds | | |
| | {close oversize brace} | 5 cycles |
| hybridization: 51° C., 50 seconds | | |
| elongation: 72° C., 1 min or 2 min and 40 sec | | |
| denaturation: 94° C., 30 seconds | | |
| | {close oversize brace} | 25 cycles |
| hybridization: 70° C., 50 seconds | | |

elongation: 72° C., 1 min or 2 min and 40 sec
72° C., 7 min
4° C.

The elongation time was 1 min for ORFs shorter than 2000 bp, and 2 min and 40 seconds for ORFs longer than 2000 bp. The amplifications were performed using a Gene Amp PCR system 9600 (Perkin Elmer).

To check the amplification results, 4 μl of each PCR product was loaded onto 1-1.5 agarose gel and the size of amplified fragments compared with DNA molecular weight standards (DNA markers III or IX, Roche). The PCR products were loaded on agarose gel and after electrophoresis the right size bands were excised from the gel. The DNA was purified from the agarose using the Gel Extraction Kit (Qiagen) following the instruction of the manufacturer. The final elution volume of the DNA was 50 μl TE (10 mM Tris-HCl, 1 mM EDTA, pH 8). One μl of each purified DNA was loaded onto agarose gel to evaluate the yield.

(E) Digestion of PCR Fragments

One-two μg of purified PCR product were double digested overnight at 37° C. with the appropriate restriction enzymes (60 units of each enzyme) using the appropriate restriction buffer in 100 μl final volume. The restriction enzymes and the digestion buffers were from New England Biolabs. After purification of the digested DNA (PCR purification Kit, Qiagen) and elution with 30 μl TE, 1 μl was subjected to agarose gel electrophoresis to evaluate the yield in comparison to titrated molecular weight standards (DNA markers III or IX, Roche).

(F) Digestion of the Cloning Vectors (pET21b+, pGEX-NN, and pGEX-NNH)

10 μg of plasmid was double digested with 100 units of each restriction enzyme in 400 μl reaction volume in the presence of appropriate buffer by overnight incubation at 37° C. After electrophoresis on a 1% agarose gel, the band corresponding to the digested vector was purified from the gel using the Qiagen Qiaex II Gel Extraction Kit and the DNA was eluted with 50 μl TE. The DNA concentration was evaluated by measuring OD₂₆₀ of the sample.

(G) Cloning

75 ng of the appropriately digested and purified vectors and the digested and purified fragments corresponding to each ORF, were ligated in final volumes of 1.0-20 μl with a molar ratio of 1:1 fragment/vector, using 400 units T4 DNA ligase (New England Biolabs) in the presence of the buffer supplied by the manufacturer. The reactions were incubated overnight at 16° C.

Transformation in E. coli DH5 competent cells was performed as follow: the ligation reaction was mixed with 200 μl of competent DH5 cells and incubated on ice for 30 min and then at 42° C. for 90 seconds. After cooling on ice, 0.8 ml LB was added and the cells were incubated for 45 min at 37° C. under shaking. 100 and 900 μl of cell suspensions were plated on separate plates of agar LB 100 μg/ml Ampicillin and the plates were incubated overnight at 37° C. The screening of the transformants was done by growing randomly chosen clones in 6 ml LB 100 μg/ml Ampicillin, by extracting the DNA using the Qiagen Qiaprep Spin Miniprep Kit following the manufacturer instructions, and by digesting 2 μl of plasmid minipreparation with the restriction enzymes specific for the restriction cloning sites. After agarose gel electrophoresis of the digested plasmid mini-preparations, positive clones were chosen on the basis of the correct size of the restriction fragments, as evaluated by comparison with appropriate molecular weight markers (DNA markers III or IX, Roche).

(H) Expression

1 μl of each right plasmid mini-preparation was transformed in 200 μl of competent E. coli strain suitable for expression of the recombinant protein. All pET21b+ recombinant plasmids were transformed in BL21 DE3 (Novagen) E. coli cells, whilst all pGEX-NN and all pGEX-NNH recombinant plasmids were transformed in BL21 cells (Novagen). After plating transformation mixtures on LB/Amp agar plates and incubation overnight at 37° C., single colonies were inoculated in 3 ml LB 100 μg/ml Ampicillin and grown at 37° C. overnight. 70 μl of the overnight culture was inoculated in 2 ml LB/Amp and grown at 37° C. until OD₆₀₀ of the pET clones reached the 0.4-0.8 value or until OD₆₀₀ of the pGEX clones reached the 0.8-1 value. Protein expression was then induced by adding IPTG (Isopropil β-D thio-galacto-piranoside) to the mini-cultures. pET clones were induced using 1 mM IPTG, whilst pGEX clones were induced using 0.2 mM IPTG. After 3 hours incubation at 37° C. the final OD₆₀₀ was checked and the cultures were cooled on ice. After centrifugation of 0.5 ml culture, the cell pellet was suspended in 50 μl of protein Loading Sample Buffer (60 mM TRIS-HCl pH 6.8, 5% w/v SDS, 10% v/v glycerin, 0.1% w/v Bromophenol Blue, 100 mM DTT) and incubated at 100° C. for 5 min. A volume of boiled sample corresponding to 0.1 OD₆₀₀ culture was analysed by SDS-PAGE and Coomassie Blue staining to verify the presence of induced protein band.

### Purification of the Recombinant Proteins

Single colonies were inoculated in 25 ml LB 100 μg/ml Ampicillin and grown at 37° C. overnight. The overnight culture was inoculated in 500 ml LB/Amp and grown under shaking at 25° C. until OD₆₀₀ 0.4-0.8 value for the pET clones, or until OD₆₀₀ 0.8-1 value for the pGEX clones. Protein expression was then induced by adding IPTG to the cultures. pET clones were induced using 1 mM IPTG, whilst pGEX clones were induced using 0.2 mM IPTG. After 4 hours incubation at 25° C. the final OD₆₀₀ was checked and the cultures were cooled on ice. After centrifugation at 6000 rpm (JA10 rotor, Beckman), the cell pellet was processed for purification or frozen at −20° C.

(I) Procedure for the Purification of Soluble His-tagged Proteins from E. coli

1. Transfer the pellets from −20° C. to ice bath and reconstitute with 10 ml 50 mM NaHPO₄ buffer, 300 mM NaCl, pH 8.0, pass in 40-50 ml centrifugation tubes and break the cells as per the following outline:

2. Break the pellets in the French Press performing three passages with in-line washing.

3. Centrifuge at about 30-40000×g per 15-20 min. If possible use rotor JA 25.50 (21000 rpm, 15 min.) or JA-20 (18000 rpm, 15 min.)

4. Equilibrate the Poly-Prep columns with 1 ml Fast Flow Chelating Sepharose resin with 50 mM phosphate buffer, 300 mM NaCl, pH 8.0.

5. Store the centrifugation pellet at −20° C., and load the supernatant in the columns.

6. Collect the flow through.

7. Wash the columns with 10 ml (2 ml+2 ml+4 ml) 50 mM phosphate buffer, 300 mM NaCl, pH 8.0.

8. Wash again with 10 ml 20 mM imidazole buffer, 50 mM phosphate, 300 mM NaCl, pH 8.0.

9. Elute the proteins bound to the columns with 4.5 ml (1.5 ml+1.5 ml+1.5 ml) 250 mM imidazole buffer, 50 mM phosphate, 300 mM NaCl, pH 8.0 and collect the 3 corresponding fractions of ˜1.5 ml each. Add to each tube 15 μl DTT 200 mM (final concentration 2 mM)

10. Measure the protein concentration of the first two fractions with the Bradford method, collect a 10 μg aliquot of proteins from each sample and analyse by SDS-PAGE. (N.B.: should the sample be too diluted, load 21 μl+7 μl loading buffer).

11. Store the collected fractions at +4° C. while waiting for the results of the SDS-PAGE analysis.

12. For immunisation prepare 4-5 aliquots of 100 μg each in 0.5 ml in 40% glycerol. The dilution buffer is the above elution buffer, plus 2 mM DTT. Store the aliquots at −20° C. until immunisation.

(J) Purification of His-tagged Proteins from Inclusion Bodies

Purifications were carried out essentially according the following protocol:

1. Bacteria are collected from 500 ml cultures by centrifugation. If required store bacterial pellets at −20° C. For extraction, resuspend each bacterial pellet in 10 ml 50 mM TRIS-HCl buffer, pH 8.5 on an ice bath.

2. Disrupt the resuspended bacteria with a French Press, performing two passages.

3. Centrifuge at 35000×g for 15 min and collect the pellets. Use a Beckman rotor JA 25.50 (21000 rpm, 15 min.) or JA-20 (18000 rpm, 15 min.).

4. Dissolve the centrifugation pellets with 50 mM TRIS-HCl, 1 mM TCEP {Tris(2-carboxyethyl)phosphine hydrochloride, Pierce), 6M guanidium chloride, pH 8.5. Stir for ˜10 min. with a magnetic bar.

5. Centrifuge as described above, and collect the supernatant.

6. Prepare an adequate number of Poly-Prep (Bio-Rad) columns containing 1 ml of Fast Flow Chelating Sepharose (Pharmacia) saturated with Nichel according to manufacturer recommendations. Wash the columns twice with 5 ml of H₂O and equilibrate with 50 mM TRIS-HCl, 1 mM TCEP, 6M guanidinium chloride, pH 8.5.

7. Load the supernatants from step 5 onto the columns, and wash with 5 ml of 50 mM TRIS-Hcl buffer, 1 mM TCEP, 6M urea, pH 8.5

8. Wash the columns with 10 ml of 20 mM imidazole, 50 mM TRIS-HCl, 6M urea, 1 mM TCEP, pH 8.5. Collect and set aside the first 5 ml for possible further controls.

9. Elute the proteins bound to the columns with 4.5 ml of a buffer containing 250 mM imidazole, 50 mM TRIS-HCl, 6M urea, 1 mM TCEP, pH 8.5. Add the elution buffer in three 1.5 ml aliquots, and collect the corresponding 3 fractions. Add to each fraction 15 μl DTT (final concentration 2 mM).

10. Measure eluted protein concentration with the Bradford method, and analyze aliquots of ca 10 μg of protein by SDS-PAGE.

11. Store proteins at −20° C. in 40% (v/v) glycerol, 50 mM TRIS-HCl, 2M urea, 0.5 M arginine, 2 mM DTT, 0.3 mM TCEP, 83.3 mM imidazole, pH 8.5

(K) Procedure for the Purification of GST-fusion Proteins from E. coli

1. Transfer the bacterial pellets from −20° C. to an ice bath and resuspend with 7.5 ml PBS, pH 7.4 to which a mixture of protease inhibitors (CØMPLETE™—Boehringer Mannheim, 1 tablet every 25 ml of buffer) has been added. Transfer to 40-50 ml centrifugation tubes and sonicate according to the following procedure:

a) Position the probe at about 0.5 cm from the bottom of the tube

b) Block the tube with the clamp

c) Dip the tube in an ice bath

d) Set the sonicator as follows: Timer→Hold, Duty Cycle→55, Out. Control→6.

e) perform 5 cycles of 10 impulses at a time lapse of 1 minute (i.e. one cycle=10 impulses+˜45″ hold; b. 10 impulses+˜45″ hold; c. 10 impulses+˜45″ hold; d. 10 impulses+˜45″ hold; e. 10 impulses+˜45″ hold)

2. Centrifuge at about 30-40000×g for 15-20 min. E.g.: use rotor Beckman JA 25.50 at 21000 rpm, for 15 min.

3. Store the centrifugation pellets at −20° C., and load the supernatants on the chromatography columns, as follows

4. Equilibrate the Poly-Prep (Bio-Rad) columns with 0.5 ml (≅1 ml suspension) of Glutathione-Sepharose 4B resin, wash with 2 ml (1+1) H₂O, and then with 10 ml (2+4+4) PBS, pH 7.4.

5. Load the supernatants on the columns and discard the flow through.

6. Wash the columns with 10 ml (2+4+4) PBS, pH 7.4.

7. Elute the proteins bound to the columns with 4.5 ml of 50 mM TRIS buffer, 10 mM reduced glutathione, pH 8.0, adding 1.5 ml+1.5 ml+1.5 ml and collecting the respective 3 fractions of ˜1.5 ml each.

8. Measure the protein concentration of the first two fractions with the Bradford method, analyse a 10 μg aliquot of proteins from each sample by SDS-PAGE. (N.B.: if the sample is too diluted load 21 μl (+7 μl loading buffer).

9. Store the collected fractions at +4° C. while waiting for the results of the SDS-PAGE analysis.

10. For each protein destined to the immunisation prepare 4-5 aliquots of 100 μg each in 0.5 ml of 40% glycerol. The dilution buffer is 50 mM TRIS.HCl, 2 mM DTT, pH 8.0. Store the aliquots at −20° C. until immunisation.

### Serology

(L) Protocol of Immunization

1. Groups of four CDI female mice aged between 6 and 7 weeks were immunized with 20 μg of recombinant protein resuspended in 100 μl.

2. Four mice for each group received 3 doses with a 14 days interval schedule.

3. Immunization was performed through intra-peritoneal injection of the protein with an equal volume of Complete Freund's Adjuvant (CFA) for the first dose and Incomplete Freund's Adjuvant (IFA) for the following two doses.

4. Sera were collected before each immunization. Mice were sacrified 14 days after the third immunization and the collected sera were pooled and stored at −20° C.

(M) Western Blot Analysis of Cpn Elementary Body Proteins with Mouse Sera

Aliquots of elementary bodies containing approximately 4 μg of proteins, mixed with SDS loading buffer (1×: 60 mM TRIS-HCl pH 6.8, 5% w/v SDS, 10% v/v glycerin, 0.1% Bromophenol Blue, 100 mM DTT) and boiled 5 minutes at 95° C., were loaded on a 12% SDS-PAGE gel. The gel was run using a SDS-PAGE running buffer containing 250 mM TRIS, 2.5 mM Glycine and 0.1% SDS. The gel was electroblotted onto nitrocellulose membrane at 200 mA for 30 minutes. The membrane was blocked for 30 minutes with PBS, 3% skimmed milk powder and incubated O/N at 4° C. with the appropriate dilution (1/100) of the sera. After washing twice with PBS+0.1% Tween (Sigma) the membrane was incubated for 2 hours with peroxidase-conjugated secondary anti-mouse antibody (Sigma) diluted 1:3000. The nitrocellulose was washed twice for 10 minutes with PBS+0.1% Tween-20 and once with PBS and thereafter developed by Opti-4CN Substrate Kit (Biorad).

Lanes shown in Western blots are: (P)=pre-immune control serum; (I)=immune serum.

(N) FACS Analysis of Chlamydia pneumoniae Elementary Bodies with Mouse Sera

1. 2×10⁵ Elementary Bodies (EB)/well were washed with 200 μl of PBS-0.1% BSA in a 96 wells U bottom plate and centrifuged for 10 min. at 1200 rpm, at 4° C.

2. The supernatant was discarded and the E.B. resuspended in 10 μl of PBS-0.01% BSA.

3. 10 μl mouse sera diluted in PBS-0.1% BSA were added to the E.B. suspention to a final dilution of 1:400, and incubated on ice for 30 min.

4. EB were washed by adding 180 μl PBS-0.1% BSA and centrifuged for 10min. at 1200 rpm, 4° C.

5. The supernatant was discarded and the E.B. resuspended in 101 of PBS-0.1% BSA.

6. 10 μl of a goat anti-mouse IgG, F(ab′)₂ fragment specific-R-Phycoerythrin-conjugated (Jackson Immunoresearch Laboratories Inc., cat.N°115-116-072) was added to the EB suspension to a final dilution of 1:100, and incubated on ice for 30 min. in the dark.

7. EB were washed by adding 180 μl PBS-0.1% BSA and centrifuged for 10 min. at 1200 rpm, 4° C.

8. The supernatant was discarded and the E.B. resuspended in 150 μl of PBS-0.1% BSA.

9. E.B. suspension was passed through a cytometric chamber of a FACS Calibur (Becton Dikinson, Mountain View, Calif. USA) and 10.000 events were acquired.

10. Data were analysed using Cell Quest Software (Becton Dikinson, Mountain View, Calif. USA) by drawing a morphological dot plot (using forward and side scatter parameters) on E.B. signals. An histogram plot was then created on FL2 intensity of fluorescence log scale recalling the morphological region of EB.

NB: the results of FACS depend not only on the extent of accessibility of the native antigens but also on the quality of the antibodies elicited by the recombinant antigens, which may have structures with a variable degree of correct folding as compared with the native protein structures. Therefore, even if a FACS assay appears negative this does not necessarily mean that the protein is not abundant or accessible on the surface. PorB antigen, for instance, gave negative results in FACS but is a surface-exposed neutralising antigen [Kubo & Stephens (2000) Mol. Microbiol. 38:772-780].

(O) Mass Spectrometry Analysis of Two-dimensional Electrophoretic Protein Maps

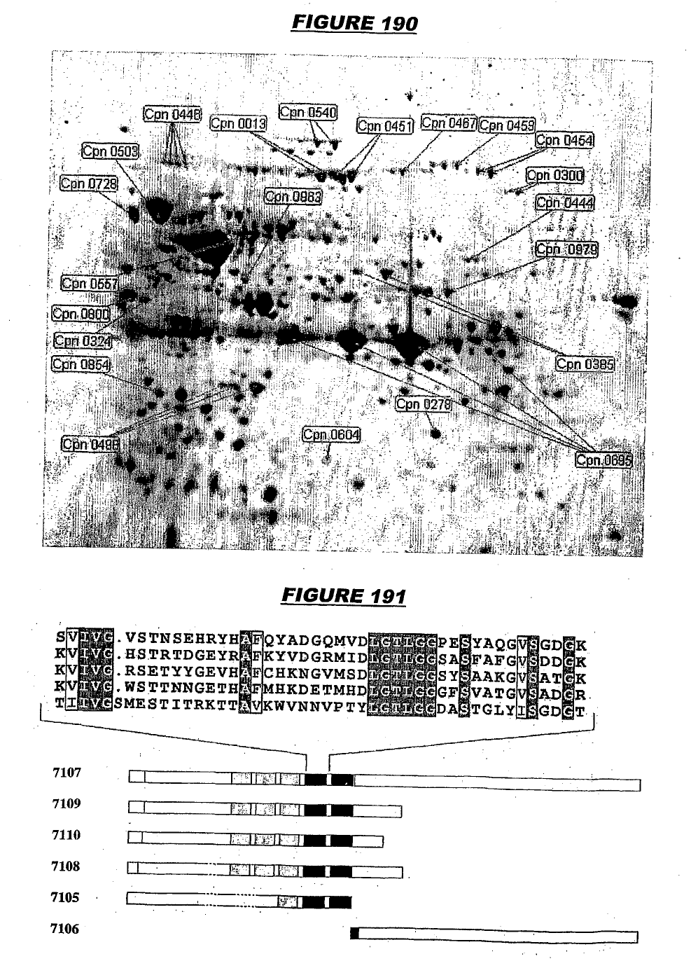

Gradient purified EBs from strain FB/96 were solubilized at a final concentration of 5.5 mg/ml with immobiline rehydratation buffer (7M urea, 2M thiourea, 2% (w/v) CHAPS, 2% (w/v) ASB 14 [Chevallet et al. (1998) Electrophor. 19:1901-9], 2% (v/v) C.A 3-10NL (Amersham Pharmacia Biotech), 2 mM tributyl phosphine, 65 mM DTT). Samples (250 μg protein) were adsorbed overnight on Immobiline DryStrips (7 cm, pH 3-10 non linear). Electrophocusing was performed in a IPGphor Isoelectric Focusing Unit (Amersham Pharmacia Biotech). Before PAGE separation, the focused strips were incubated in 4M urea, 2M thiourea, 30% (v/v) glycerol, 2% (w/v) SDS, 5 mM tributyl phosphine 2.5%(w/v) acrylamide, 50 mM Tris-HCl pH 8.8, as described [Herbert et al. (1998) Electrophor. 19:845-51]. SDS-PAGE was performed on linear 9-16% acrylamide gradients. Gels were stained with colloidal Coomassie (Novex, San Diego) [Doherty et al. (1998) Electrophor. 19:355-63]. Stained gels were scanned with a Personal Densitometer SI (Molecular Dynamics) at 8 bits and 50 μm per pixel. Map images were annotated with the software Image Master 2D Elite, version 3.10 (Amersham Pharmacia Biotech). Protein spots were excised from the gel, using an Ettan Spot picker (Amersham Pharmacia Biotech), and dried in a vacuum centrifuge. In-gel digestion of samples for mass spectrometry and extraction of peptides were performed as described by Wilm et al. [Nature (1996) 379:466-9]. Samples were desalted with a ZIP TIP (Millipore), eluted with a saturated solution of alpha-cyano-4-hydroxycinnamic acid in 50% acetonitrile, 0.1% TFA and directly loaded onto a SCOUT 381 multiprobe plate (Bruker). Spectra were acquired on a Bruker Biflex II MALDI-TOF. Spectra were calibrated using a combination of known standard peptides, located in spots adjacent to the samples. Resulting values for monoisotopic peaks were used for database searches using the computer program Mascot (www.matrixscience.com). All searches were performed using an error of 200-500 ppm as constraint. A representative gel is shown in FIG. 190.

### Example 1

The following C. pneumoniae protein (PID 4376552) was expressed <SEQ ID 1; cp6552>:

| | |
|---|---|
| 1 | MKKKLSLLVG LIFVLSSCHK EDAQNKIRIV ASPTPHAELL ESLQEEAKDL |
| 51 | GIKLKILPVD DYRIPNRLLL DKQVDANYFQ HQAFLDDECE RYDCKGELVV |
| 101 | IAKVHLEPQA IYSKKHSSLE RLKSQKKLTI AIPVDRTNAQ RALHLLEECG |
| 151 | LIVCKGPANL NMTAKDVCGK ENRSINILEV SAPLLVGSLP DVDAAVIPGN |
| 201 | FAIAANLSPK KDSLCLEDLS VSKYTNLVVI RSEDVGSPKM IKLQKLFQSP |
| 251 | SVQHFFDTKY HGNILTMTQD NG* |

A predicted signal peptide is highlighted.

The cp6552 nucleotide sequence <SEQ ID 2> is:

| | |
|---|---|
| 1 | ATGAAAAAAA AATTATCATT ACTTGTAGGT TTAATTTTTG TTTTGAGTTC |
| 51 | TTGCCATAAG GAAGATGCTC AGAATAAAAT ACGTATTGTA GCCAGTCCGA |
| 101 | CACCTCATGC GGAATTATTG GAGAGTTTAC AGGAAGAGGC TAAAGATCTT |
| 151 | GGAATCAAGC TGAAAATACT TCCAGTAGAT GATTATCGTA TTCCTAATCG |
| 201 | TTTGCTTTTG GATAAACAAG TAGATGCAAA TTACTTTCAA CATCAAGCTT |
| 251 | TTCTTGATGA CGAATGCGAG CGTTATGATT GTAAGGGTGA ATTAGTTGTT |
| 301 | ATCGCTAAAG TTCATTTGGA ACCTCAAGCA ATTTATTCTA AGAAACATTC |
| 351 | TTCTTTAGAG CGCTTAAAAA GCCAGAAGAA ACTGACTATA GCGATTCCTG |
| 401 | TGGATCGTAC GAATGCTCAG CGTGCTCTAC ACTTGTTAGA AGAGTGCGGA |
| 451 | CTCATTGTTT GCAAAGGGCC TGCTAATTTA AATATGACAG CTAAAGATGT |
| 501 | CTGTGGGAAA GAAAATAGAA GTATCAACAT ATTAGAGGTG TCAGCTCCTC |
| 551 | TTCTTGTCGG ATCTCTTCCT GACGTTGATG CTGCTGTCAT TCCTGGAAAT |
| 601 | TTTGCTATAG CAGCAAACCT TTCTCCAAAG AAAGATAGTC TTTGTTTAGA |
| 651 | GGATCTTTCG GTATCTAAGT ATACAAACCT TGTTGTCATT CGTTCTGAAG |
| 701 | ACGTAGGTTC TCCTAAAATG ATAAAATTAC AGAAGCTGTT TCAATCTCCT |
| 751 | TCTGTACAAC ATTTTTTTGA TACAAAATAT CATGGGAATA TTTTGACAAT |
| 801 | GACTCAAGAC AATGGTTAG |

The PSORT algorithm predicts an inner membrane location (0.127).

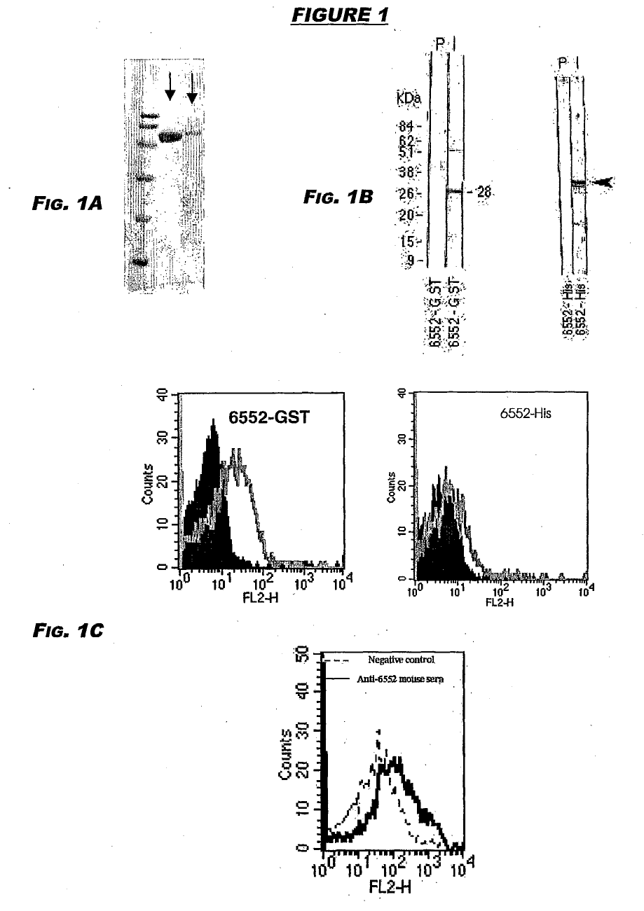

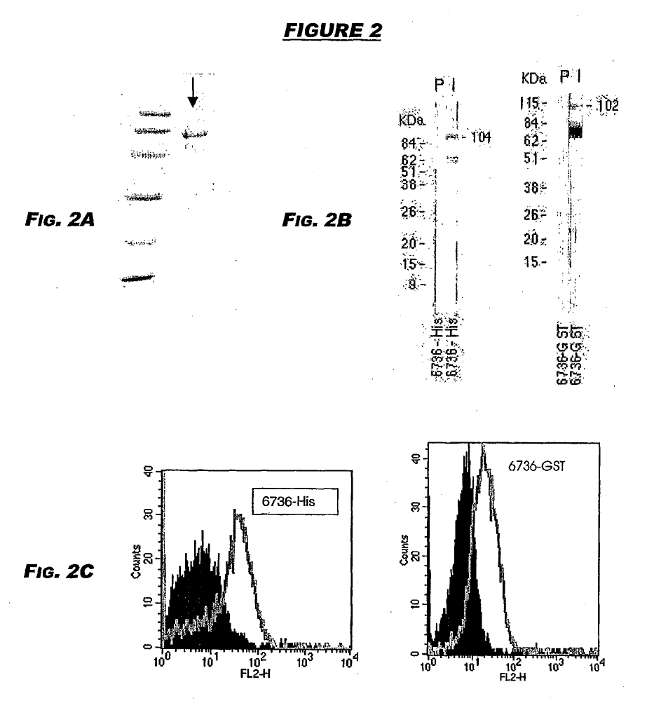

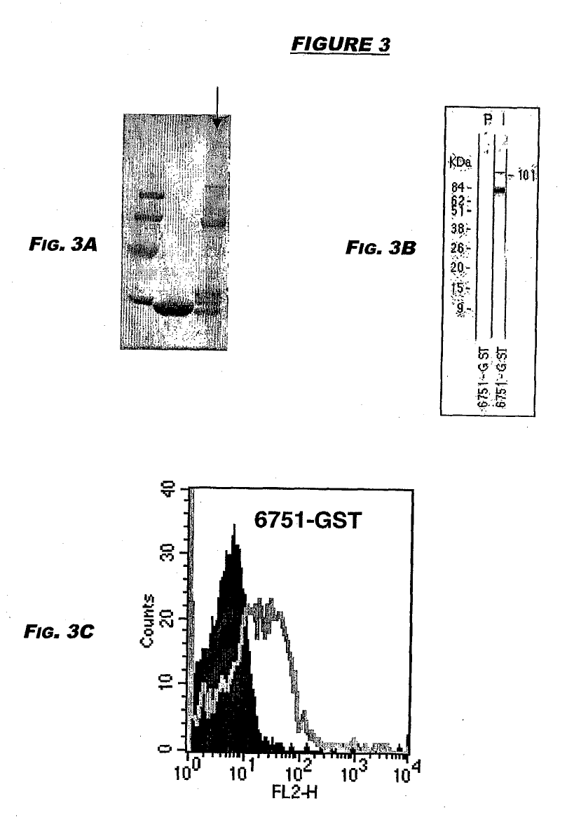

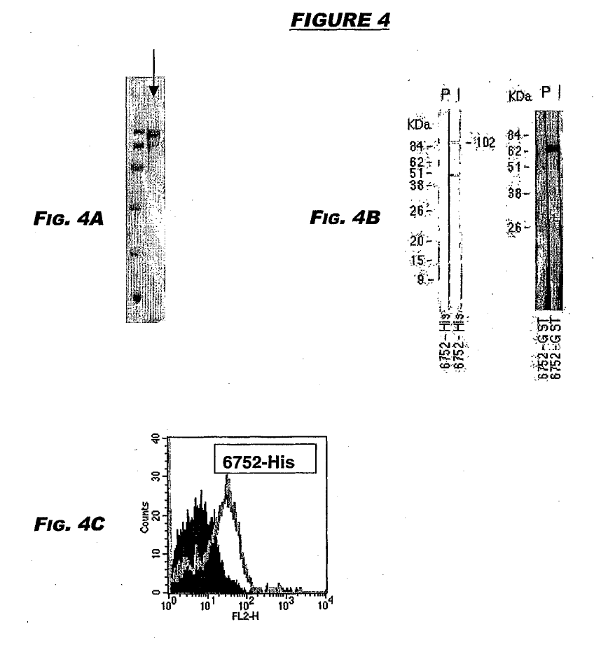

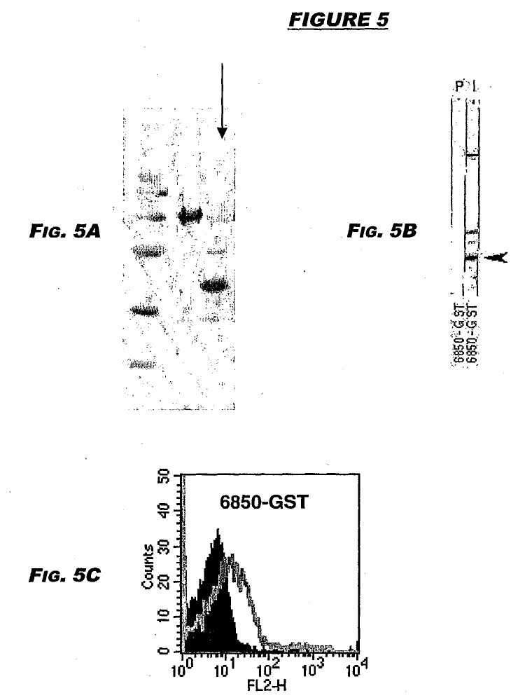

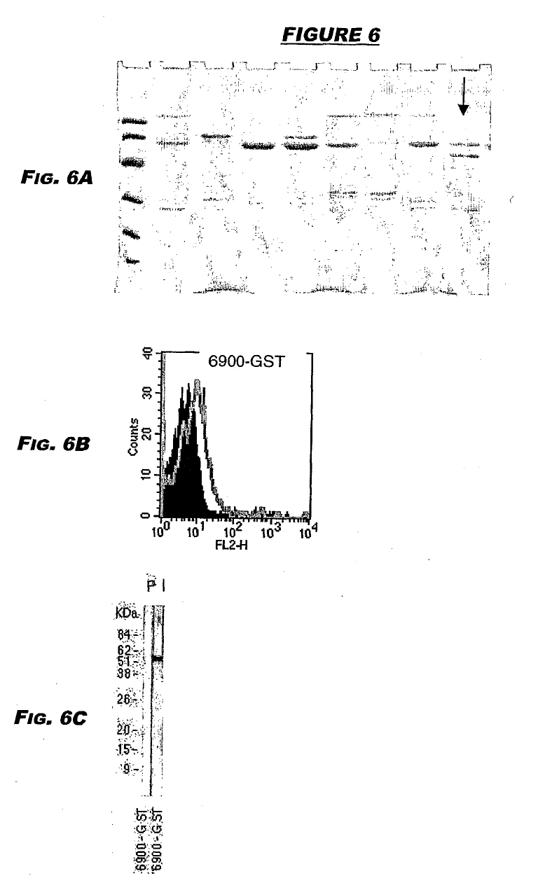

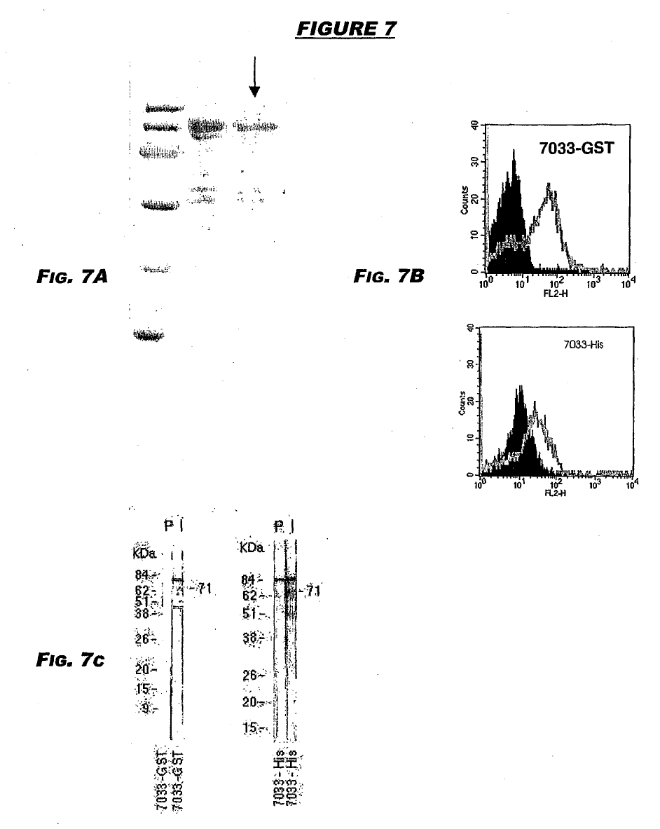

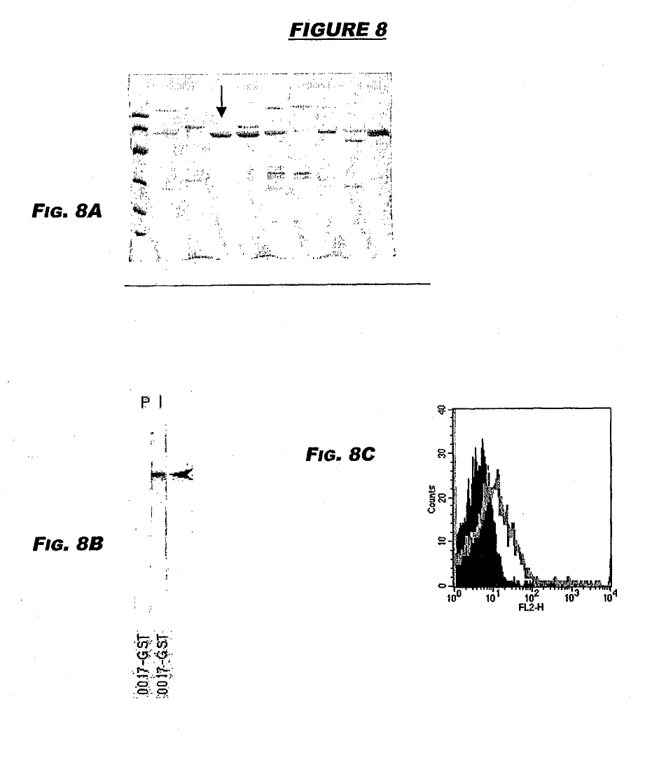

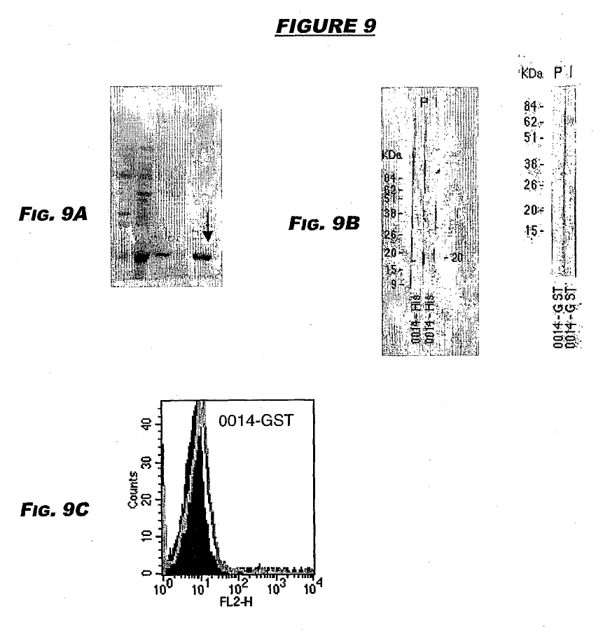

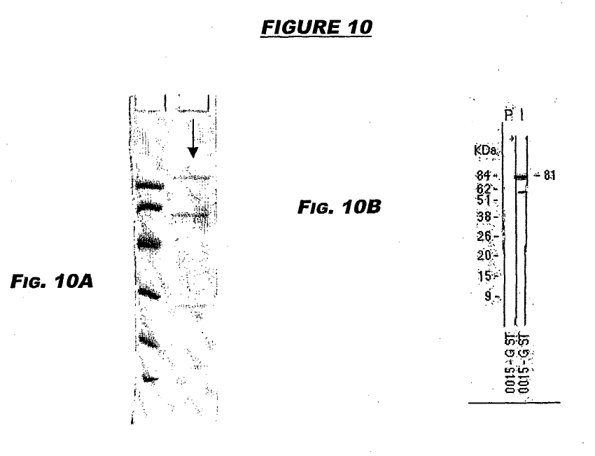

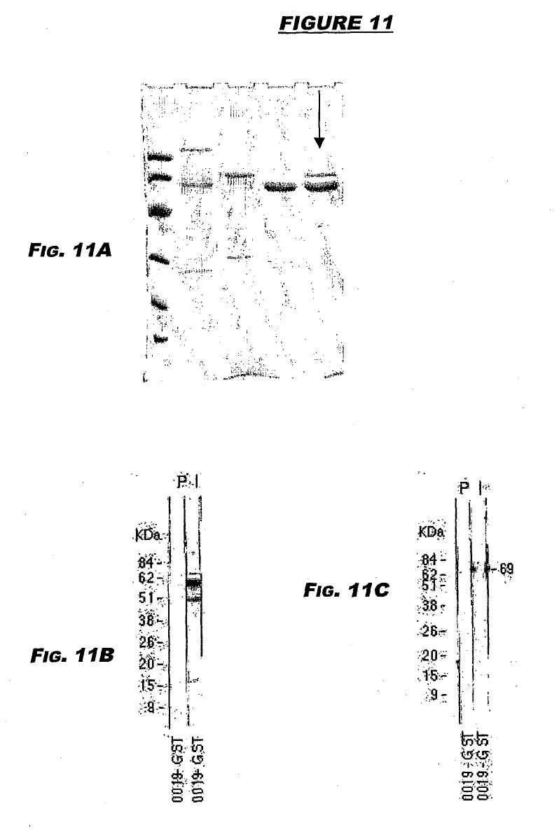

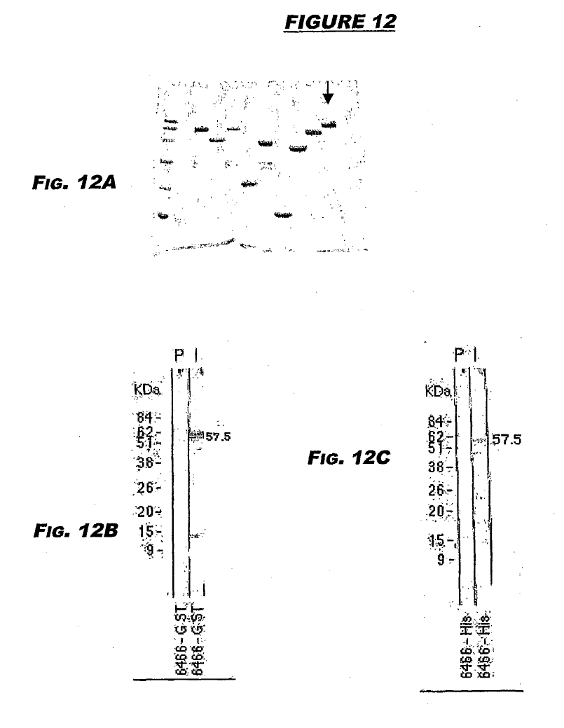

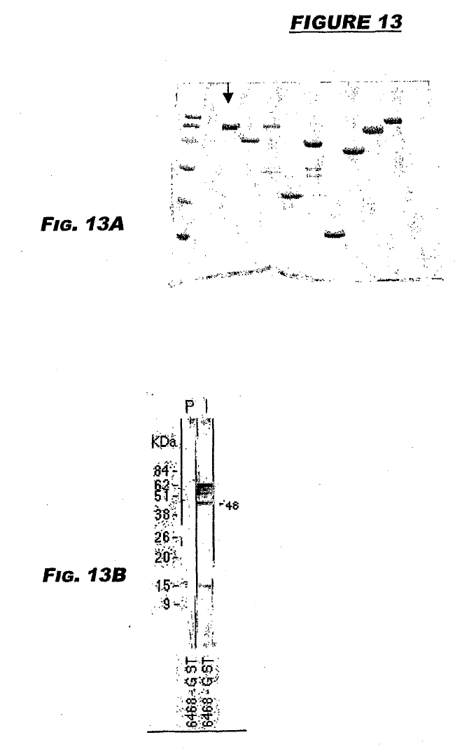

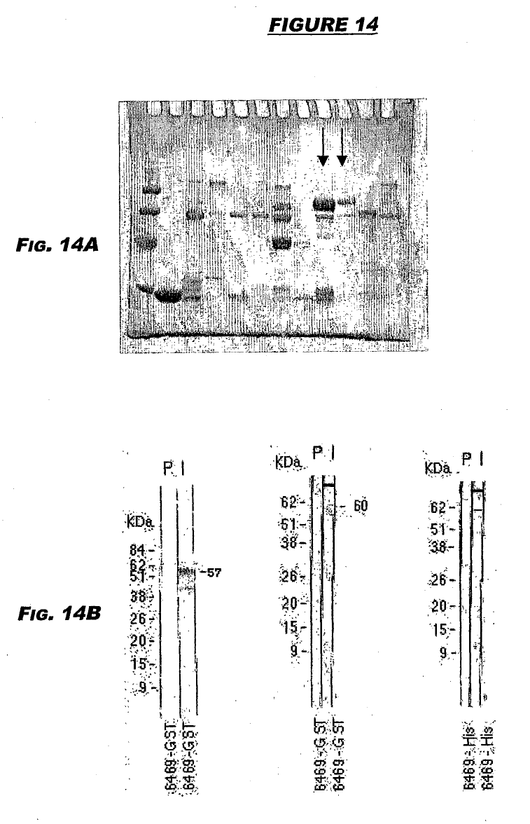

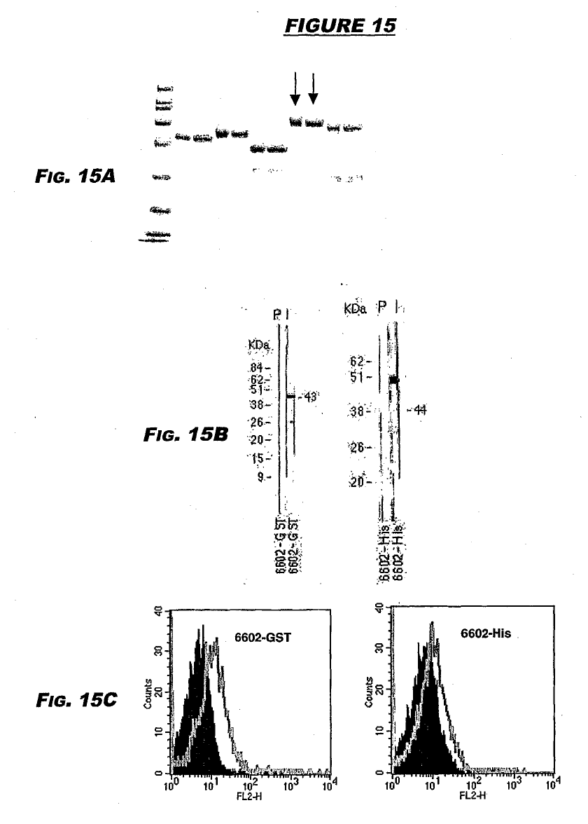

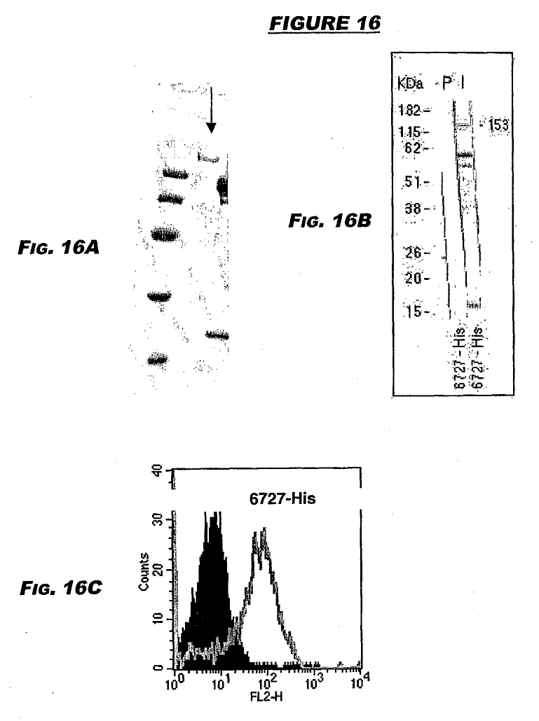

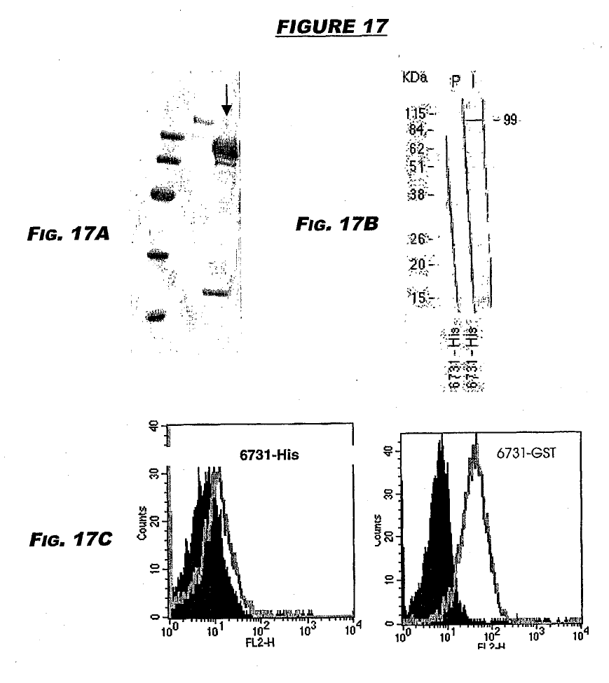

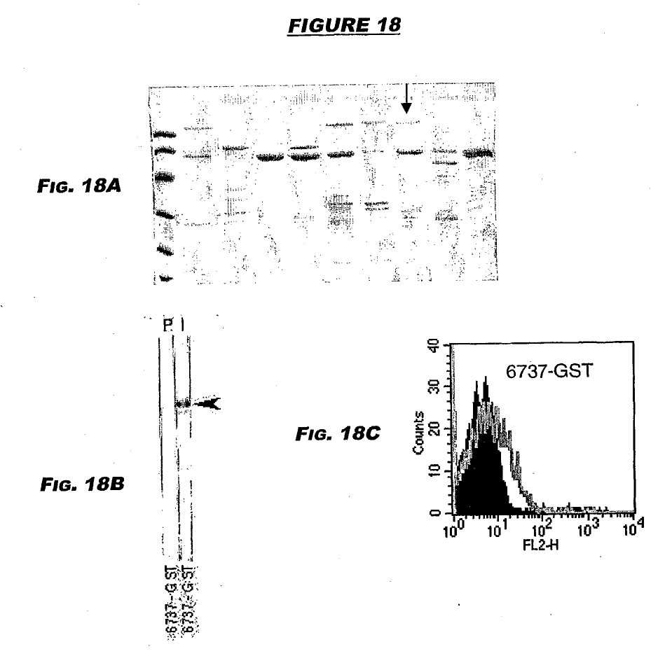

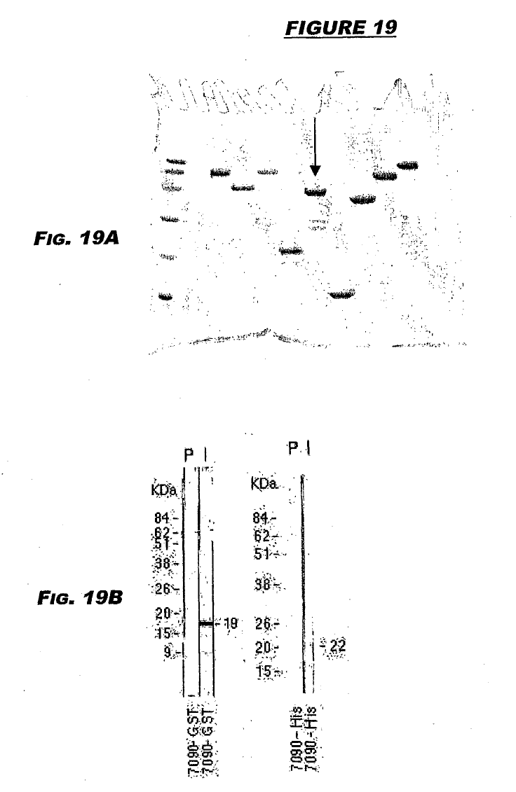

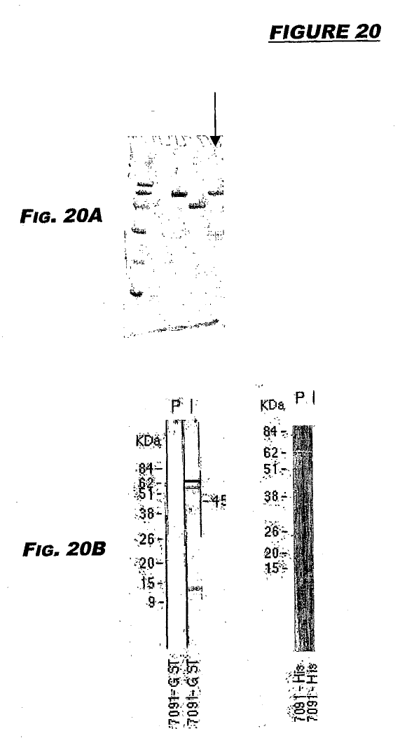

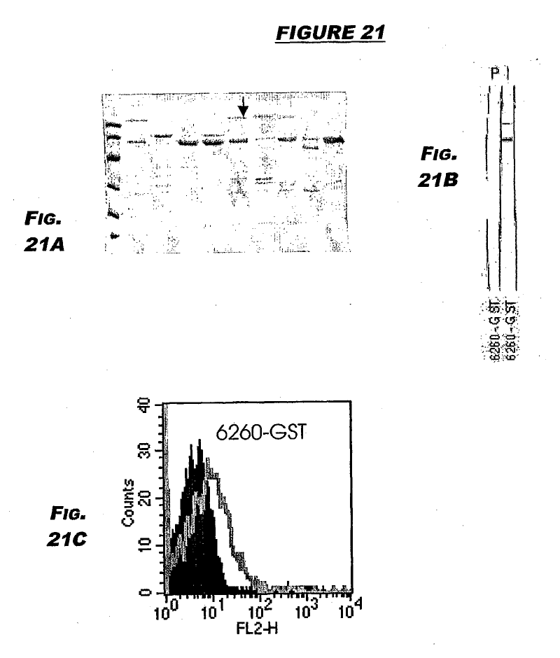

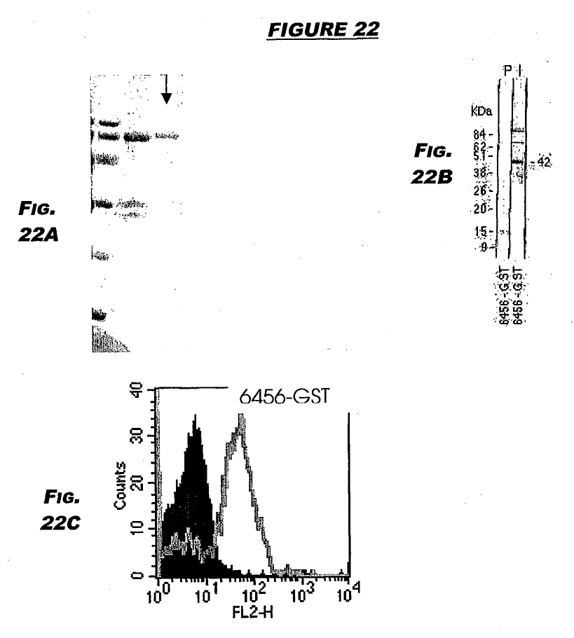

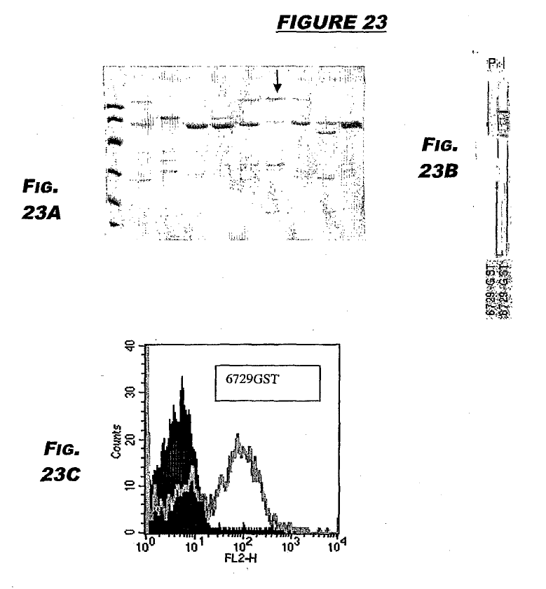

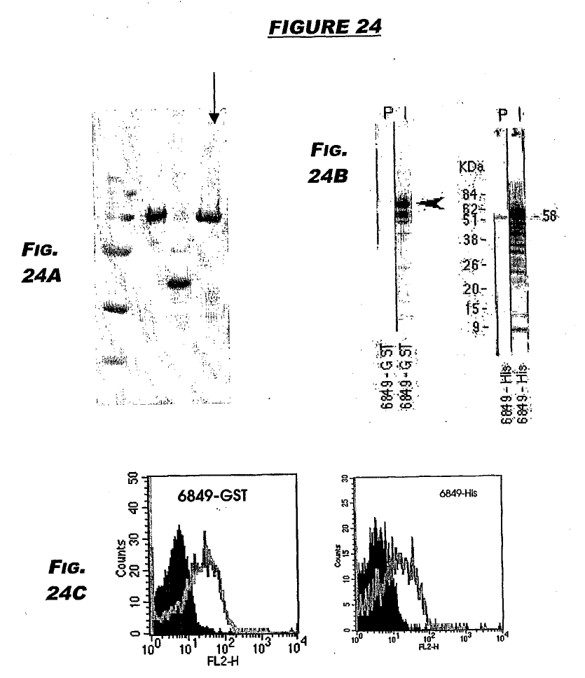

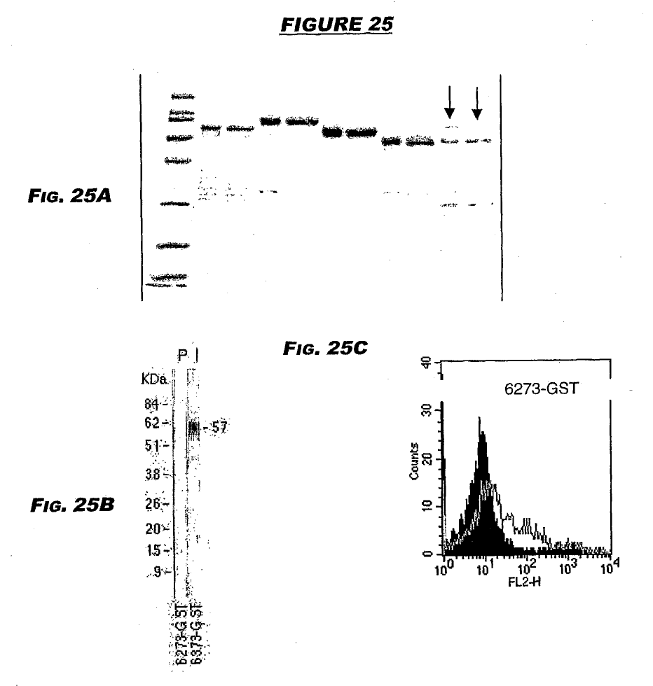

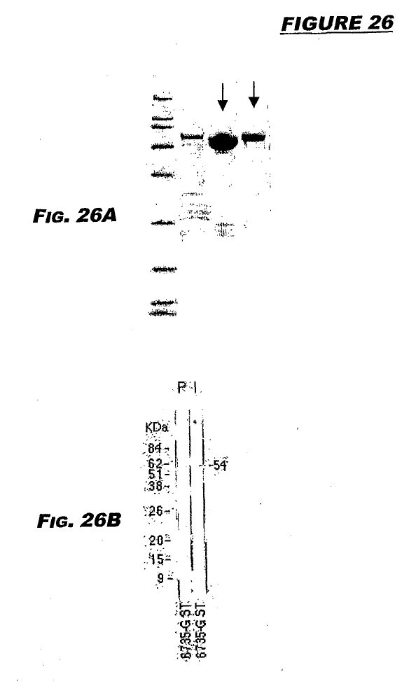

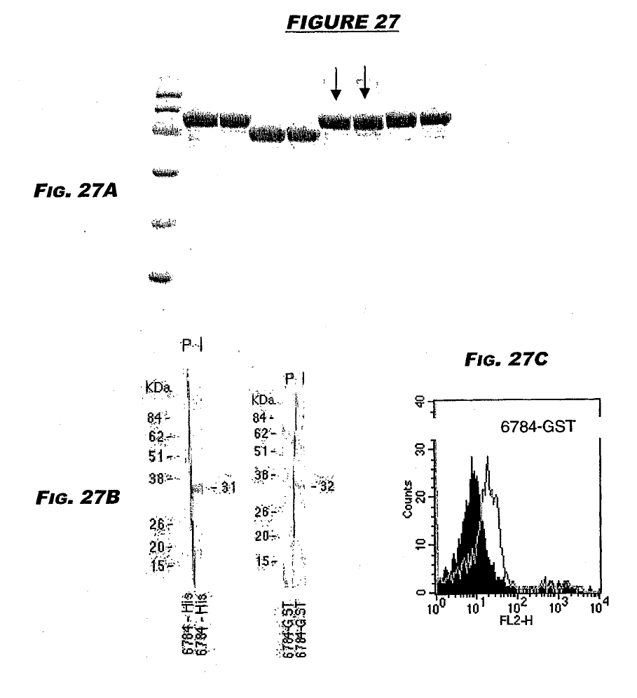

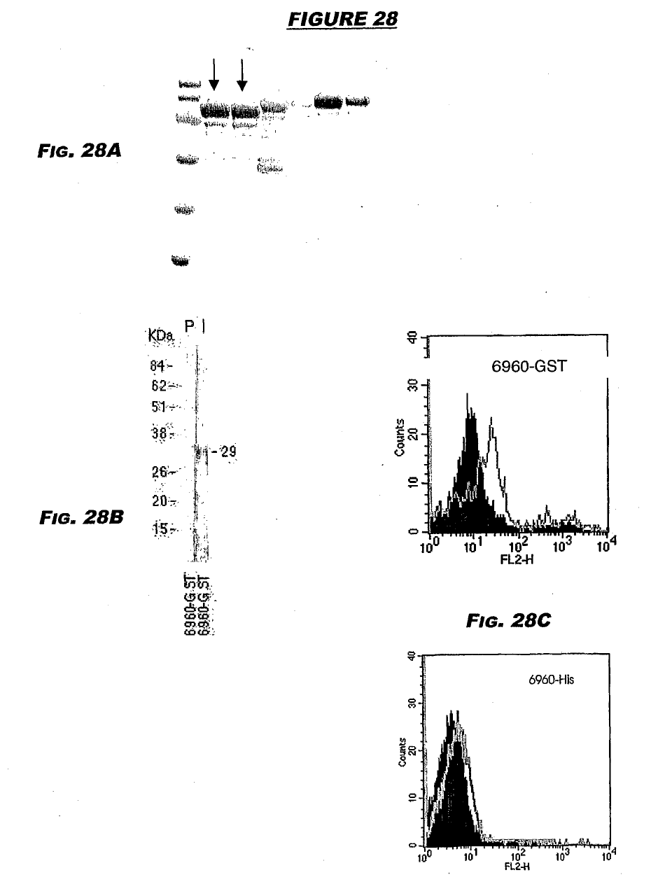

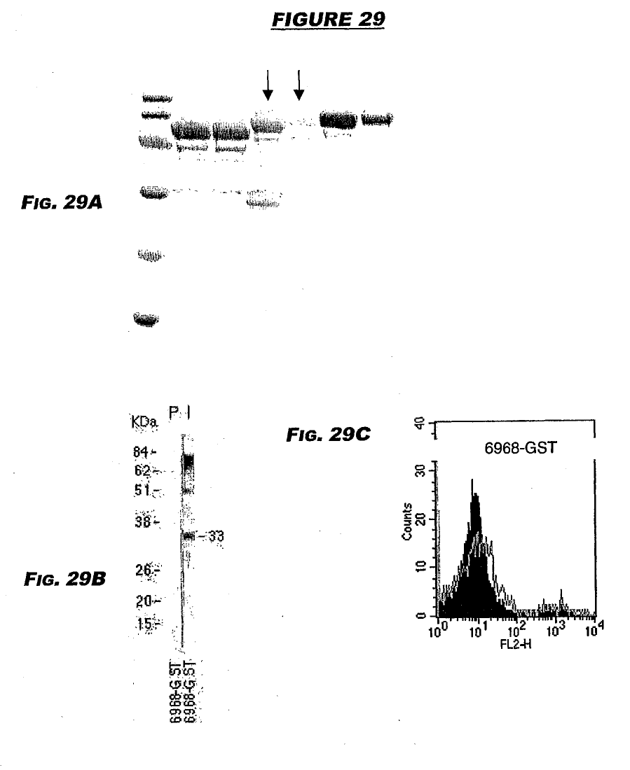

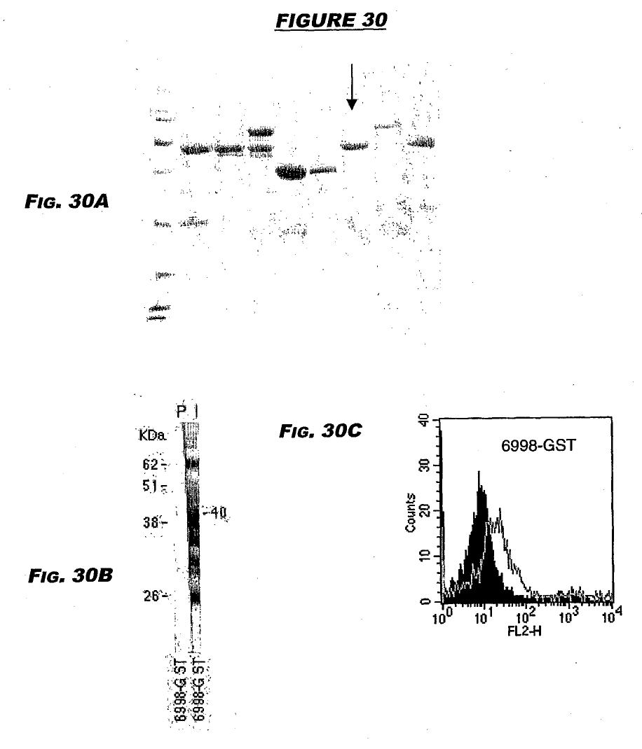

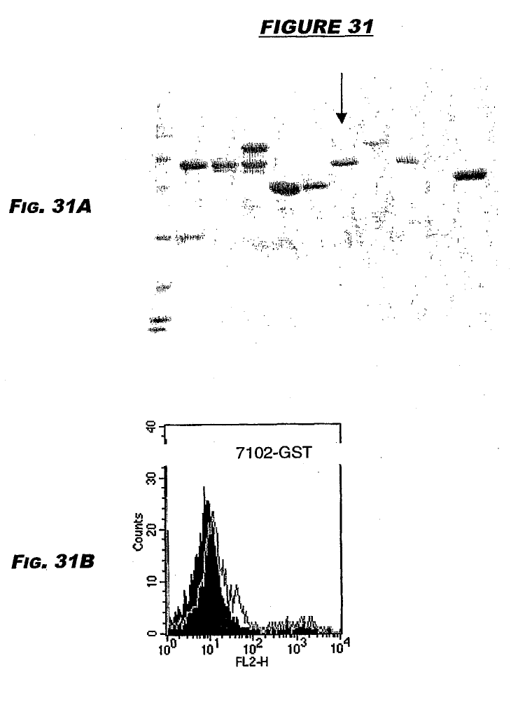

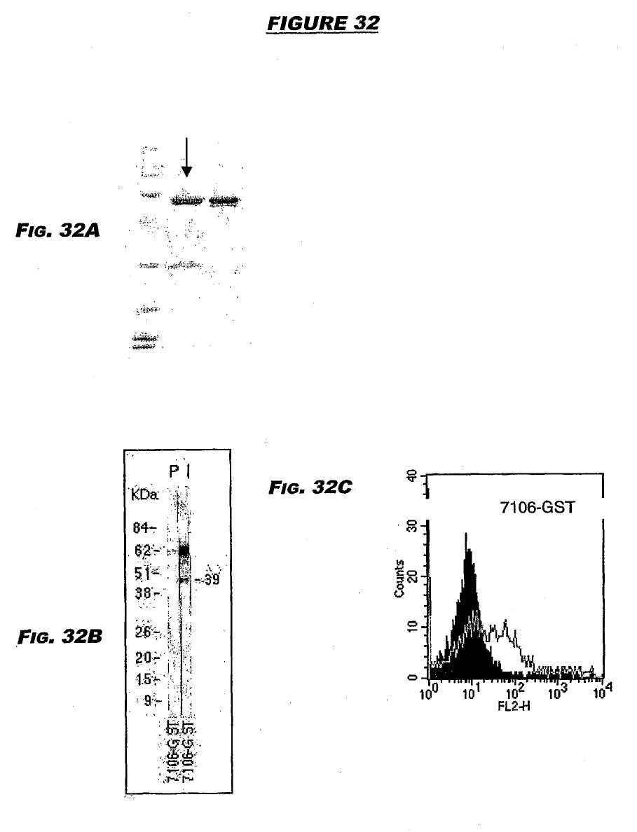

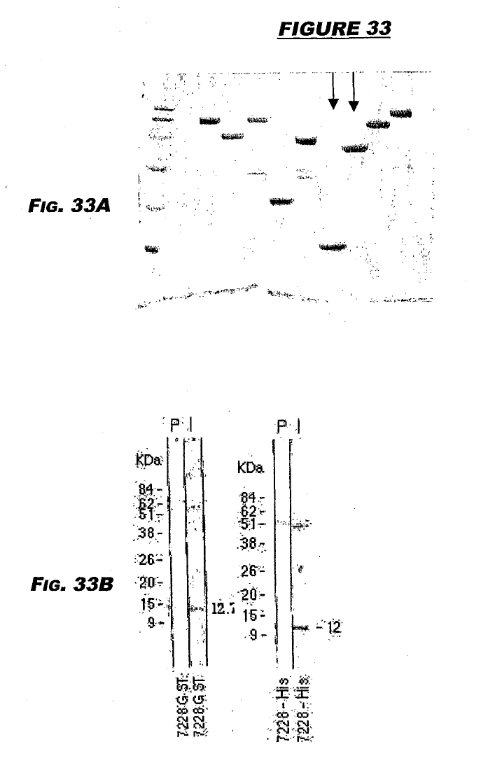

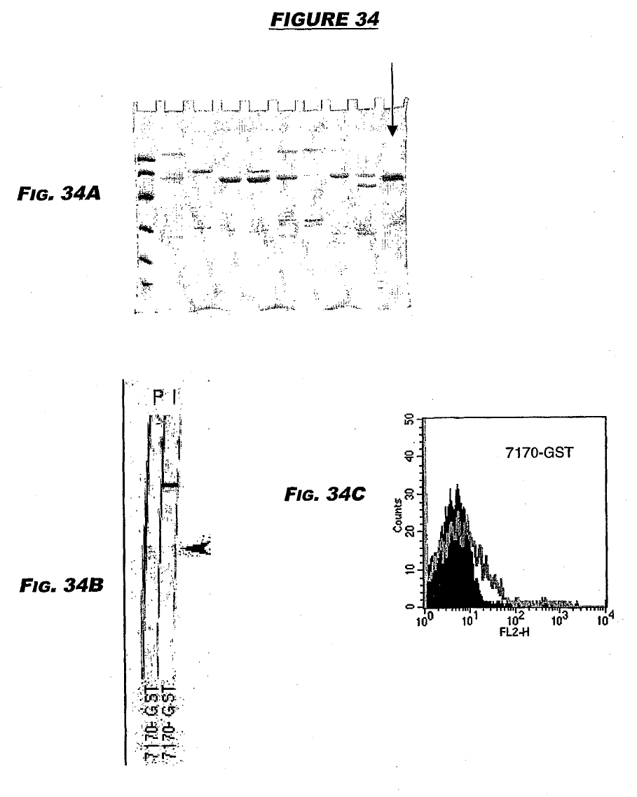

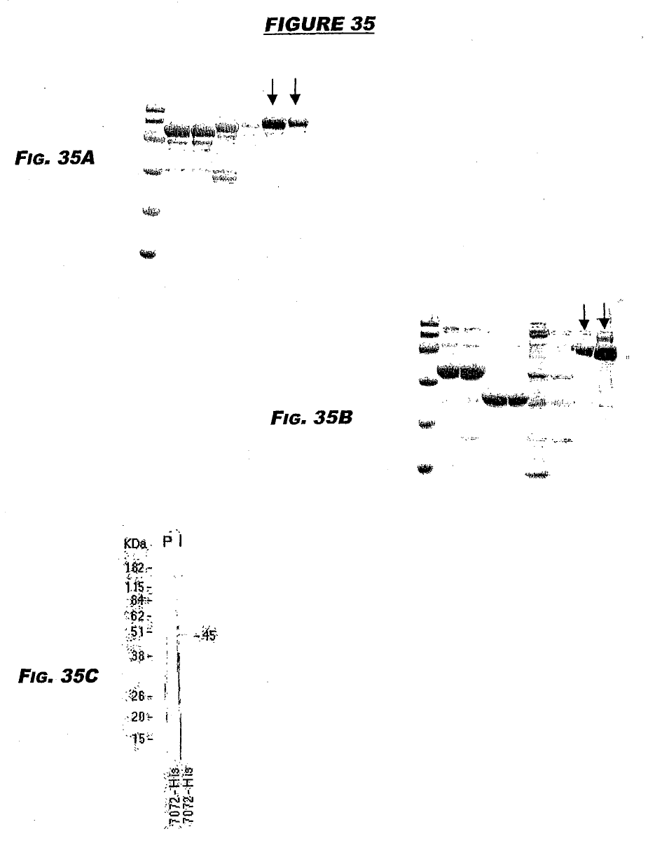

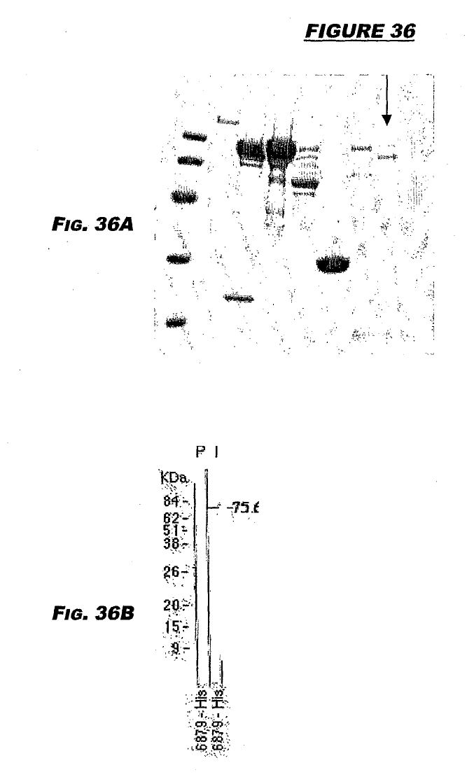

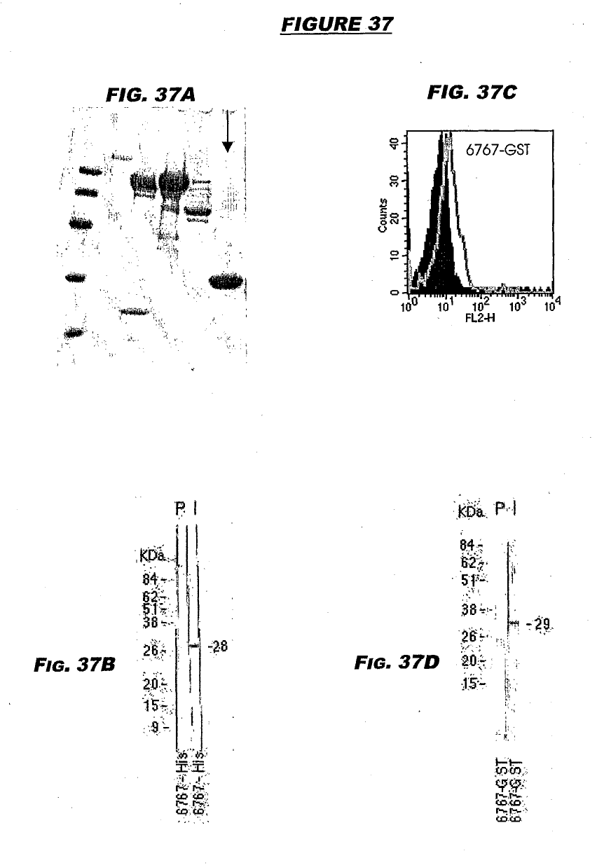

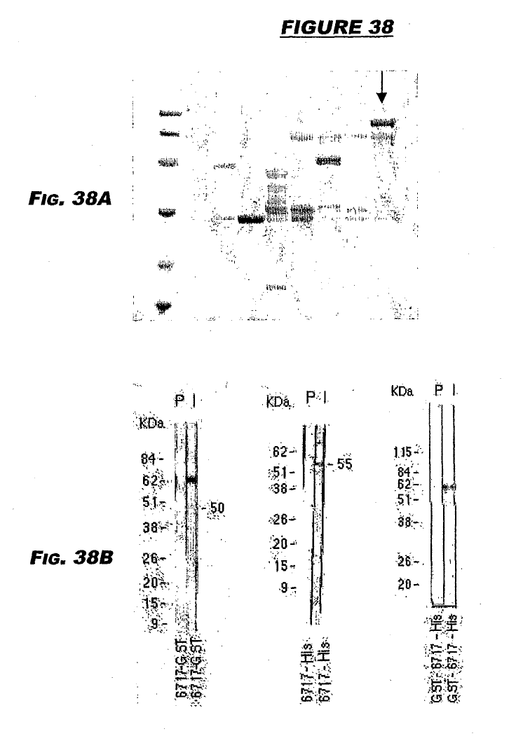

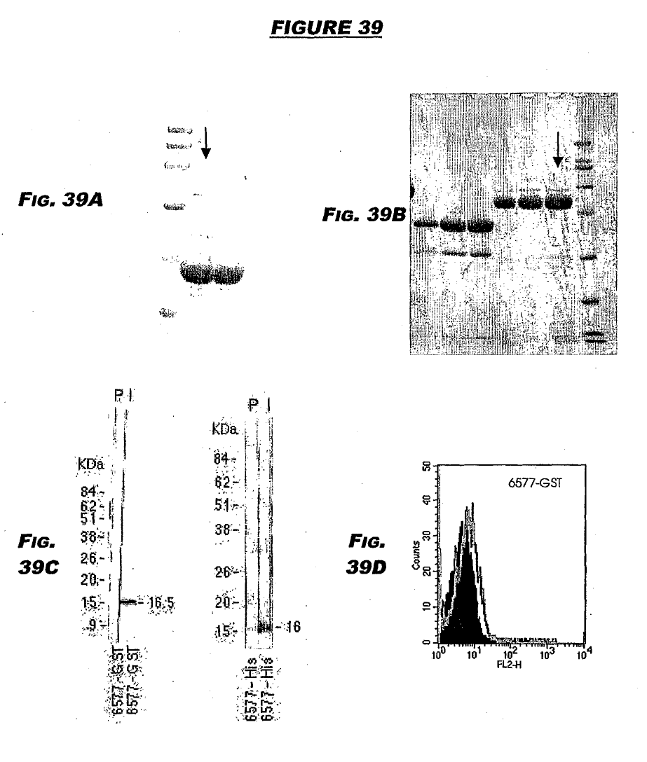

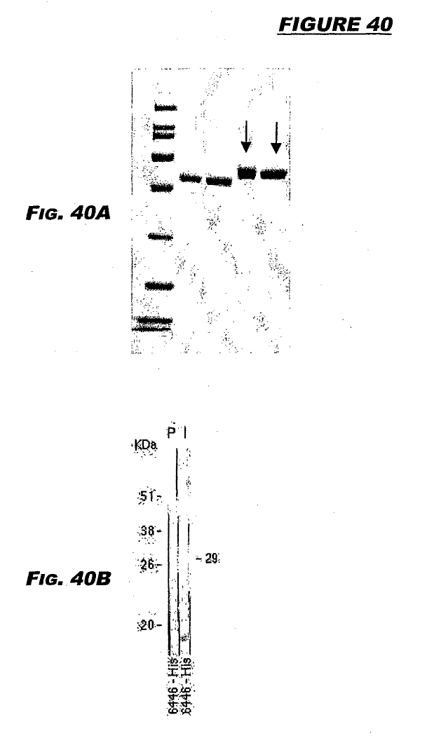

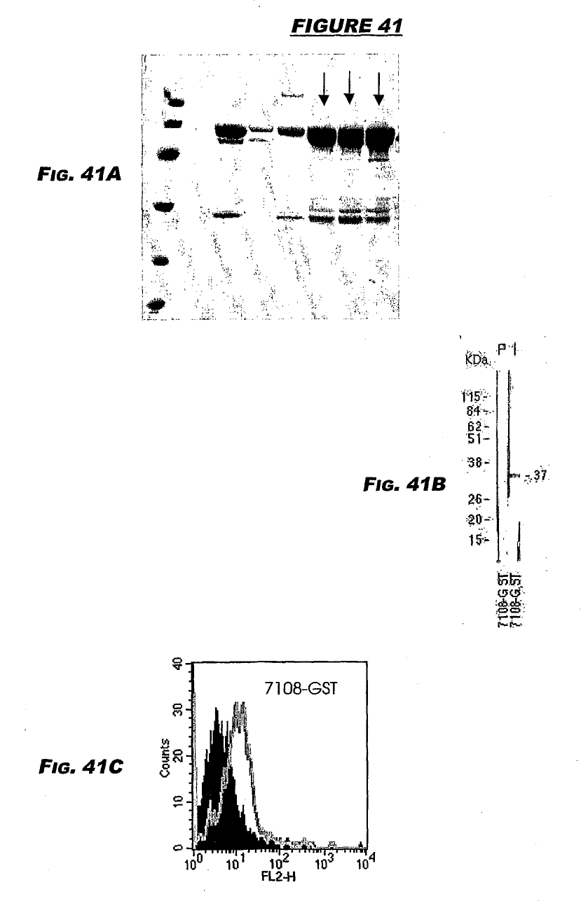

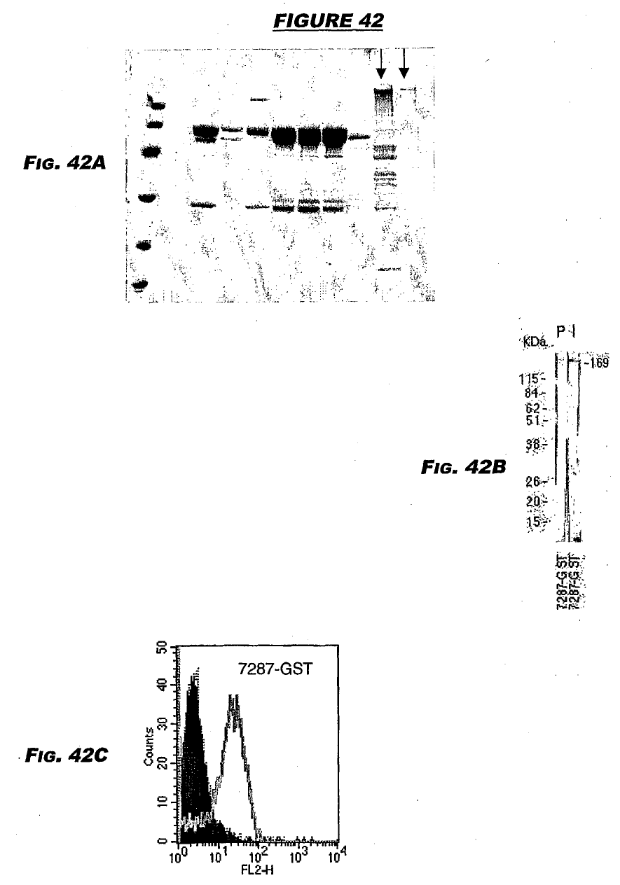

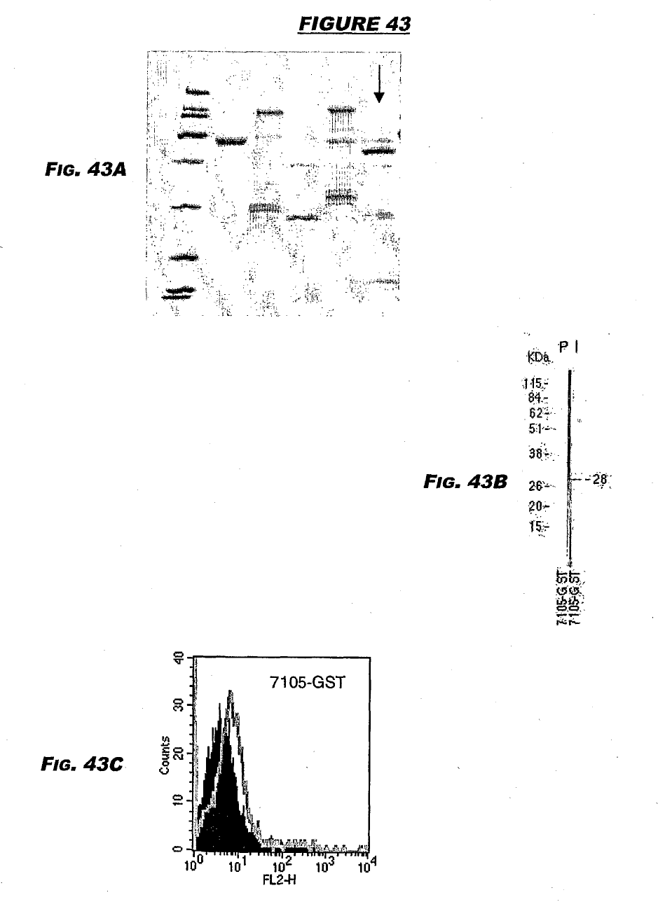

The protein was expressed in E. coli and purified as a his-tag product, as shown in FIG. 1A, and also as a GST-fusion. The recombinant protein was used to immunise mice, whose sera were used in a Western blot (FIG. 1B) and for FACS analysis (FIG. 1C).

The cp6552 protein was also identified in the 2D-PAGE experiment (Cpn0278).

These experiments show that cp6552 is a surface-exposed and immunoaccessible protein, and that it is a useful immunogen. These properties are not evident from the sequence alone.

### Example 2

The following C. pneumoniae protein (PID 4376736) was expressed <SEQ ID 3; cp6736>:

| | |
|---|---|
| 1 | MKTSIRKFLI STTLAPCFAS TAFTVEVIMP SENFDGSSGK IFPYTTLSDP |
| 51 | RGTLCIFSGD LYIANLDNAI SRTSSSCFSN RAGALQILGK GGVFSFLNIR |
| 101 | SSADGAAISS VITQNPELCP LSFSGFSQMI FDNCESLTSD TSASNVIPHA |
| 151 | SAIYATTPML FTNNDSILFQ YNRSAGFGAA IRGTSITIEN TKKSLLFNGN |
| 201 | GSISNGGALT GSAAINLINN SAPVIFSTNA TGIYGGAIYL TGGSMLTSGN |
| 251 | LSGVLFVNNS SRSGGAIYAN GNVTFSNNSD LTFQNNTASP QNSLPAPTPP |
| 301 | PTPPAVTPLL GYGGAIFCTP PATPPPTGVS LTISGENSVT FLENIASEQG |
| 351 | GALYGKKISI DSNKSTIFLG NTAGKGGAIA IPESGELSLS ANQGDILFNK |
| 401 | NLSITSGTPT RNSIHPGKDA KFATLGATQG YTLYFYDPIT SDDLSAASAA |
| 451 | ATVVVNPKAS ADGAYSGTIV FSGETLTATE AATPANATST LNQKLELEGG |
| 501 | TLALRNGATL NVHNFTQDEK SVVIMDAGTT LATTNGANNT DGAITLNKLV |
| 551 | INLDSLDGTK AAVVNVQSTN GALTISGTLG LVKNSQDCCD NHGMFNKDLQ |
| 601 | QVPILELKAT SNTVTTTDFS LGTNGYQQSP YGYQGTWEFT IDTTTHTVTG |
| 651 | NWKKTGYLPH PERLAPLIPN SLWANVIDLR AVSQASAADG EDVPGKQLSI |
| 701 | TGITNFFHAN HTGDARSYRH MGGGYLINTY TRITPDAALS LGFGQLFTKS |
| 751 | KDYLVGHGHS NVYPAPVYSN ITKSLFGSSR FFSGGTSRVT YSRSNEKVKT |
| 801 | SYTKLPKGRC SWSNNCWLGE LEGNLPITLS SRILNLKQII PFVKAEVAYA |
| 851 | THGGIQENTP EGRIFGHGHL LNVAVPVGVR FGKNSHNRPD FYTIIVAYAP |
| 901 | DVYRHNPDCD TTLPINGATW TSIGNNLTRS TLLVQASSHT SVNDVLEIFG |
| 951 | HCGCDIRRTS RQYTLDIGSK LRF* |

A predicted signal peptide is highlighted.

The cp6736 nucleotide sequence <SEQ ID 4> is:

| | |
|---|---|
| 1 | ATGAAAACGT CTATTCGTAA GTTCTTAATT TCTACCACAC TGGCGCCATG |
| 51 | TTTTGCTTCA ACAGCGTTTA CTGTAGAAGT TATCATGCCT TCCGAGAACT |
| 101 | TTGATGGATC GAGTGGGAAG ATTTTTCCTT ACACAACACT TTCTGATCCT |
| 151 | AGAGGGACAC TCTGTATTTT TTCAGGGGAT CTCTACATTG CCAATCTTGA |
| 201 | TAATGCCATA TCCAGAACCT CTTCCAGTTG CTTTAGCAAT AGGGCGGGAG |
| 251 | CACTACAAAT CTTAGGAAAA GGTGGGGTTT TCTCCTTCTT AAATATCCGT |
| 301 | TCTTCAGCTG ACGGAGCCGC GATTAGTAGT GTAATCACCC AAAATCCTGA |
| 351 | ACTATGTCCC TTGAGTTTTT CAGGATTTAG TCAGATGATC TTCGATAACT |
| 401 | GTGAATCTTT GACTTCAGAT ACCTCAGCGA GTAATGTCAT ACCTCACGCA |
| 451 | TCGGCGATTT ACGCTACAAC GCCCATGCTC TTTACAAACA ATGACTCCAT |
| 501 | ACTATTCCAA TACAACCGTT CTGCAGGATT TGGAGCTGCC ATTCGAGGCA |
| 551 | CAAGCATCAC AATAGAAAAT ACGAAAAAGA GCCTTCTCTT TAATGGTAAT |
| 601 | GGATCCATCT CTAATGGAGG GGCCCTCACG GGATCTGCAG CGATCAACCT |
| 651 | CATCAACAAT AGCGCTCCTG TGATTTTCTC AACGGGATCT ACAGGGATCT |
| 701 | ATGGTGGGGC TATTTACCTT ACCGGAGGAT CTATGCTCAC CTCTGGGAAC |
| 751 | CTCTCAGGAG TCTTGTTCGT TAATAATAGC TCGCGCTCAG GAGGCGCTAT |
| 801 | CTATGCTAAC GGAAATGTCA CATTTTCTAA TAACAGCGAC CTGACTTTCC |
| 851 | AAAACAATAC AGCATCTCCA CAAAACTCCT TACCTGCACC TACACCTCCA |
| 901 | CCTACACCAC CAGCAGTCAC TCCTTTGTTA GGATATGGAG GCGCCATCTT |
| 951 | CTGTACTCCT CCAGCTACCC CCCCACCAAC AGGTGTTAGC CTGACTATAT |
| 1001 | CTGGAGAAAA CAGCGTTACA TTCCTAGAAA ACATTGCCTC CGAACAAGGA |
| 1051 | GGAGCCCTCT ATGGCAAAAA GATCTCTATA GATTCTAATA AATCTACAAT |
| 1101 | ATTTCTTGGA AATACAGCTG GAAAAGGAGG CGCTATTGCT ATTCCCGAAT |
| 1151 | CTGGGGAGCT CTCTCTATCC GCAAATCAAG GTGATATCCT CTTTAACAAG |
| 1201 | AACCTCAGCA TCACTAGTGG GACACCTACT CGCAATAGTA TTCACTTCGG |
| 1251 | AAAAGATGCC AAGTTTGCCA CTCTAGGAGC TACGCAAGGC TATACCCTAT |
| 1301 | ACTTCTATGA TCCGATTACA TCTGATGATT TATCTGCTGC ATCCGCAGCC |
| 1351 | GCTACTGTGG TCGTCAATCC CAAAGCCAGT GCAGATGGTG CGTATTCAGG |
| 1401 | GACTATTGTC TTTTCAGGAG AAACCCTCAC TGCTACCGAA GCAGCAACCC |
| 1451 | CTGCAAATGC TACATCTACA TTAAACCAAA AGCTAGAACT TGAAGGCGGT |
| 1501 | ACTCTCGCTT TAAGAAACGG TGCTACCTTA AATGTTCATA ACTTCACGCA |
| 1551 | AGATGAAAAG TCCGTCGTCA TCATGGATGC AGGGACCACA TTAGCAACTA |
| 1601 | CAAATGGAGC TAATAATACT GACGGTGCTA TCACCTTAAA CAAGCTTGTA |
| 1651 | ATCAATCTGG ATTCTTTGGA TGGCACTAAA GCGGCTGTCG TTAATGTGCA |
| 1701 | GAGTACCAAT GGAGCTCTCA CTATATCCGG AACTTTAGGA CTTGTGAAAA |
| 1751 | ACTCTCAAGA TTGCTGTGAC AACCACGGGA TGTTTAATAA AGATTTACAG |
| 1801 | CAAGTTCCGA TTTTAGAACT CAAAGCGACT TCAAATACTG TAACCACTAC |
| 1851 | GGACTTCAGT CTCGGCACAA ACGGCTATCA GCAATCTCCC TATGGGTATC |
| 1901 | AAGGAACTTG GGAGTTTACC ATAGACACGA CAACCCATAC GGTCACAGGA |
| 1951 | AATTGGAAAA AAACCGGTTA TCTTCCTCAT CCGGAGCGTC TTGCTCCCCT |
| 2001 | CATTCCTAAT AGCCTATGGG CAAACGTCAT AGATTTACGA GCTGTAAGTC |
| 2051 | AAGCGTCAGC AGCTGATGGC GAAGATGTCC CTGGGAAGCA ACTGAGCATC |
| 2101 | ACAGGAATTA CAAATTTCTT CCATGCGAAT CATACCGGTG ATGCACGCAG |
| 2151 | CTACCGCCAT ATGGGTGGAG GCTACCTCAT CAATACCTAC ACACGCATCA |
| 2201 | CTCCAGATGC TGCGTTAAGT CTAGGTTTTG GACAGCTGTT TACAAAATCT |
| 2251 | AAGGATTACC TCGTAGGTCA CGGTCATTCT AACGTTTATT TCGCTACAGT |
| 2301 | ATACTCTAAC ATCACCAAGT CTCTGTTTGG ATCATCGAGA TTCTTCTCAG |
| 2351 | GAGGCACTTC TCGAGTTACC TATAGCCGTA GCAATGAGAA AGTAAAGACT |
| 2401 | TCATATACAA AATTGCCTAA AGGGCGCTGC TCTTGGAGTA ACAATTGCTG |
| 2451 | GTTAGGAGAA CTCGAAGGGA ACCTTCCCAT CACTCTCTCT TCTCGCATCT |
| 2501 | TAAACCTCAA GCAGATCATT CCCTTTGTAA AAGCTGAAGT TGCTTACGCG |
| 2551 | ACTCATGGGG GCATCCAAGA AAATACCCCC GAGGGGAGGA TTTTTGGACA |
| 2601 | CGGTCATCTA CTCAACGTTG CAGTTCCCGT AGGCGTCCGC TTTGGTAAAA |
| 2651 | ATTCTCATAA TCGACCAGAT TTTTACACTA TAATCGTAGC CTATGCTCCT |
| 2701 | GATGTCTATC GTCACAATCC TGATTGCGAT ACGACATTAC CTATTAATGG |
| 2751 | AGCTACGTGG ACCTCTATAG GGAATAATCT AACCAGAAGT ACTTTGCTAG |
| 2801 | TACAAGCATC CAGCCATACT TCAGTAAATG ATGTTCTAGA GATCTTCGGG |
| 2851 | CACTGTGGAT GTGATATTCG CAGAACCTCC CGTCAATATA CTCTAGATAT |
| 2901 | AGGAAGCAAA TTACGATTTT AA |

The PSORT algorithm predicts an outer membrane location (0.917).

The protein was expressed in E. coli and purified as a his-tag product, as shown in FIG. 2A, and also as a GST-fusion. Both proteins were used to immunise mice, whose sera were used in a Western blot (FIG. 2B) and for FACS analysis (FIG. 2C).

The cp6736 protein was also identified in the 2D-PAGE experiment (Cpn0453) and showed good cross-reactivity with human sera, including sera from patients with pneumonitis.

These experiments show that cp6736 is a surface-exposed and immunoaccessible protein, and that it is a useful immunogen. These properties are not evident from the sequence alone.

### Example 3

The following C. pneumoniae protein (PID 4376751) was expressed <SEQ ID 5; cp6751>:

| | |
|---|---|
| 1 | MRFFCFGMLL PFTFVLANEG LQLPLETYIT LSPEYQAAPQ VGFTHNQNQD |
| 51 | LAIVGNHNDF ILDYKYYRSN GGALTCKNLL ISENIGNVFF EKNVCPNSGG |
| 101 | AIYAAQNCTI SKNQNYAFTT NLVSDNPTAT AGSLLGGALF AINCSITNNL |
| 151 | GQGTFVDNLA LNKGGALYTE TNLSIKDNKG PIIIKQNRAL NSDSLGGGIY |
| 201 | SGNSLNIEGN SGAIQITSNS SGSGGGIFST QTLTISSNKK LIEISENSAF |
| 251 | ANNYGSNFNP GGGGLTTTFC TILNNREGVL FNNNQSQSNG GAIHAKSIII |
| 301 | KENGPVYFLN NTATRGGALL NLSAGSGNGS FILSADNGDI IFNNNTASKH |
| 351 | ALNPPYRNAI HSTPNMNLQI GARPGYRVLF YDPIEHELPS SFPILFNFET |
| 401 | GHTGTVLFSG EHVHQNFTDE MNFFSYLRNT SELRQGVLAV EDGAGLAQYK |
| 451 | FFQRGGTLLL GQGAVITTAG TIPTPSSTPT TVGSTITLNH IAIDLPSILS |
| 501 | FQAQAPKIWI YPTKTGSTYT EDSNPTITIS GTLTLRNSNN EDPYDSLDLS |
| 551 | HSLEKVPLLY IVDVAAQKIN SSQLDLSTLN SGEHYGYQGI WSTYWVETTT |
| 601 | ITNPTSLLGA NTKHKLLYAN WSPLGYRPHP ERRGEFITNA LWQSAYTALA |
| 651 | GLHSLSSWDE EKGHAASLQG IGLLVHQKDK NGFKGFRSHM TGYSATTEAT |
| 701 | SSQSPNFSLG FAQFFSKAKE HESQNSTSSH HYFSGMCIEN TLFKEWIRLS |
| 751 | VSLAYMFTSE HTHTMYQGLL EGNSQGSFHN HTLAGALSCV PLPQPHGESL |
| 801 | QIYPFITALA IRGNLAAFQE SGDHAREFSL HRPLTDVSLP VGIRASWKNH |
| 851 | HRVPLVWLTE ISYRSTLYRQ DPELHSKLLI SQGTWTTQAT PVTYNALGIK |
| 901 | VKNTMQVFPK VTLSLDYSAD ISSSTLSHYL NVASRMRF* |

A predicted signal peptide is highlighted.

The cp6751 nucleotide sequence <SEQ ID 6> is:

| | |
|---|---|
| 1 | ATGCGCTTTT TTTGCTTCGG AATGTTGCTT CCTTTTACTT TTGTATTGGC |
| 51 | TAATGAAGGT CTCCAACTTC CTTTGGAGAC CTATATTACA TTAAGTCCTG |
| 101 | AATATCAAGC AGCCCCTCAA GTAGGGTTTA CTCATAACCA AAATCAAGAT |
| 151 | CTCGCAATTG TCGGGAATCA CAATGATTTC ATCTTGGACT ATAAGTACTA |
| 201 | TCGGTCGAAT GGAGGTGCTC TTACCTGTAA GAATCTTCTG ATCTCTGAAA |
| 251 | ATATAGGGAA TGTCTTCTTT GAGAAGAATG TCTGTCCCAA TTCTGGCGGG |
| 301 | GCAATTTATG CTGCTCAAAA TTGCACGATC TCCAAGAATC AGAACTATGC |
| 351 | ATTTACTACA AACTTGGTCT CTGACAATCC TACAGCCACT GCGGGATCAC |
| 401 | TATTGGGTGG AGCTCTCTTT GCCATAAATT GCTCTATTAC TAATAACCTA |
| 451 | GGACAGGGAA CTTTCGTTGA CAATCTCGCT TTAAATAAGG GGGGTGCCCT |
| 501 | CTATACTGAG ACGAACTTAT CTATTAAAGA CAATAAAGGC CCGATCATAA |
| 551 | TCAAGCAGAA TCGGGCACTA AATTCGGACA GTTTAGGAGG AGGGATTTAT |
| 601 | AGTGGGAACT CTCTAAATAT AGAGGGAAAT TCTGGAGCTA TACAGATCAC |
| 651 | AAGCAACTCT TCAGGATCTG GGGGAGGCAT ATTTTCTACC CAAACACTCA |
| 701 | CGATCTCCTC GAATAAAAAA CTCATAGAAA TCAGTGAAAA TTCCGCGTTC |
| 751 | GCAAATAACT ATGGATCGAA CTTCAATCCA GGAGGAGGAG GTCTTACTAC |
| 801 | CACCTTTTGC ACGATATTGA ACAACCGAGA AGGGGTACTC TTTAACAATA |
| 851 | ACCAAAGCCA GAGCAACGGT GGAGCCATTC ATGCGAAATC TATCATTATC |
| 901 | AAAGAAAATG GTCCTGTATA CTTTTTAAAT AACACTGCAA CTCGGGGAGG |
| 951 | GGCTCTCCTC AACTTATCAG CAGGTTCTGG AAACGGAAGC TTCATCTTAT |
| 1001 | CTGCAGATAA TGGAGATATT ATCTTTAACA ATAATACGGC CTCCAAGCAT |
| 1051 | GCCCTCAATC CTCCATACAG AAACGCCATT CACTCGACTC CTAATATGAA |
| 1101 | TCTGCAAATA GGAGCCCGTC CCGGCTATCG AGTGCTGTTC TATGATCCCA |
| 1151 | TAGAACATGA GCTCCCTTCC TCCTTCCCCA TACTCTTTAA TTTCGAAACC |
| 1201 | GGTCATACAG GTACAGTTTT ATTTTCAGGG GAACATGTAC ACCAGAACTT |
| 1251 | TACCGATGAA ATGAATTTCT TTTCCTATTT AAGGAACACT TCGGAACTAC |
| 1301 | GTCAAGGAGT CCTTGCTGTT GAAGATGGTG CGGGGCTGGC CTGCTATAAG |
| 1351 | TTCTTCCAAC GAGGAGGCAC TCTACTTCTA GGTCAAGGTG CGGTGATCAC |
| 1401 | GACAGCAGGA ACGATTCCCA CACCATCCTC AACACCAACG ACAGTAGGAA |
| 1451 | GTACTATAAC TTTAAATCAC ATTGCCATTG ACCTTCCTTC TATTCTTTCT |
| 1501 | TTTCAAGCTC AGGCTCCAAA AATTTGGATT TACCCCACAA AAACAGGATC |
| 1551 | TACCTATACT GAAGATTCCA ACCCGACAAT CACAATCTCA GGAACTCTCA |
| 1601 | CCTTACGCAA CAGCAACAAC GAAGATCCCT ACGATAGTCT GGATCTCTCG |
| 1651 | CACTCTCTTG AGAAAGTTCC CCTTCTTTAT ATTGTCGATG TCGCTGCACA |
| 1701 | AAAAATTAAC TCTTCGCAAC TGGATCTATC CACATTAAAT TCTGGCGAAC |
| 1751 | ACTATGGGTA TCAAGGCATC TGGTCGACCT ATTGGGTAGA AACTACAACA |
| 1801 | ATCACGAACC CTACATCTCT ACTAGGCGCG AATACAAAAC ACAAGCTGCT |
| 1851 | CTATGCAAAC TGGTCTCCTC TAGGCTACCG TCCTCATCCC GAACGTCGAG |
| 1901 | GAGAATTCAT TACGAATGCC TTGTGGCAAT CGGCATATAC GGCTCTTGCA |
| 1951 | GGACTCCACT CCCTCTCCTC CTGGGATGAA GAGAAGGGTC ATGCAGCTTC |
| 2001 | CCTACAAGGC ATTGGTCTTC TGGTTCATCA AAAAGACAAA AACGGTTTTA |
| 2051 | AGGGATTTCG TAGTCATATG ACAGGTTATA GTGCTACCAC CGAAGCAACC |
| 2101 | TCTTCTCAAA GTCCGAATTT CTCTTTAGGA TTTGCTCAGT TCTTCTCCAA |
| 2151 | AGCTAAAGAA CATGAATCTC AAAATAGCAC GTCCTCTCAC CACTATTTCT |
| 2201 | CTGGAATGTG CATAGAAAAT ACTCTCTTCA AAGAGTGGAT ACGTCTATCT |
| 2251 | GTGTCTCTTG CTTATATGTT TACCTCGGAA CATACCCATA CAATGTATCA |
| 2301 | GGGTCTCCTG GAAGGGAACT CTCAGGGATC TTTCCACAAC CATACCTTAG |
| 2351 | CAGGGGCTCT CTCCTGTGTT TTCTTACCTC AACCTCACGG CGAGTCCCTG |
| 2401 | CAGATCTATC CCTTTATTAC TGCCTTAGCC ATCCGAGGAA ATCTTGCTGC |
| 2451 | GTTTCAAGAA TCTGGAGACC ATGCTCGGGA ATTTTCCCTA CACCGCCCCC |
| 2501 | TAACGGACGT CTCCCTCCCT GTAGGAATCC GCGCTTCTTG GAAGAACCAC |
| 2551 | CACCGAGTTC CCCTAGTCTG GCTCACAGAA ATTTCCTATC GCTCTACTCT |
| 2601 | CTATAGGCAA GATCCTGAAC TCCACTCGAA ATTACTGATT AGCCAAGGTA |
| 2651 | CGTGGACGAC GCAGGCCACT CCTGTGACCT ACAATGCTTT AGGGATCAAA |
| 2701 | GTGAAAAATA CCATGCAGGT GTTTCCTAAA GTCACTCTCT CCTTAGATTA |
| 2751 | CTCTGCGGAT ATTTCTTCCT CCACGCTGAG TCACTACTTA AACGTGGCGA |
| 2801 | GTAGAATGAG ATTTTAA |

The PSORT algorithm predicts an outer membrane location (0.923).

The protein was expressed in E. coli and purified as a GST-fusion product, as shown in FIG. 3A, and also in his-tagged form. The GST-fusion recombinant protein was used to immunise mice, whose sera were used in a Western blot (FIG. 3B) and for FACS analysis (FIG. 3C).

This protein also showed good cross-reactivity with human sera, including sera from patients with pneumonitis.

These experiments show that cp6751 is a surface-exposed and immunoaccessible protein, and that it is a useful immunogen. These properties are not evident from the sequence alone.

### Example 4

The following C. pneumoniae protein (PID 4376752) was expressed <SEQ ID 7; cp6752>:

| | |
|---|---|
| 1 | MFGMTPAVYS LQTDSLEKFA LERDEEFRTS FPLLDSLSTL TGFSPITTFV |
| 51 | GNRHNSSQDI VLSNYKSIDN ILLLWTSAGG AVSCNNFLLS NVEDHAFFSK |
| 101 | NLAIGTGGAI ACQGACTITK NRGPLIFFSN RGLNNASTGG ETRGGAIACN |
| 151 | GDFTISQNQG TFYFVNNSVN NWGGALSTNG HCRIQSNRAP LLFFNNTAPS |
| 201 | GGGALRSENT TISDNTRPIY FKNNCGNNGG AIQTSVTVAI KNNSGSVIFN |
| 251 | NNTALSGSIN SGNGSGGAIY TTNLSIDDNP GTILFNNNYC IRDGGAICTQ |
| 301 | FLTIKNSGHV YFTNNQGNWG GALMLLQDST CLLFAEQGNI AFQNNEVFLT |
| 351 | TFGRYNAIHC TPNSNLQLGA NKGYTTAFFD PIEHQHPTTN PLIFNPNANH |
| 401 | QGTILFSSAY IPEASDYENN FISSSKNTSE LRNGVLSIED RAGWQFYKFT |
| 451 | QKGGILKLGH AASIATTANS ETPSTSVGSQ VIINNLAINL PSILARGKAP |
| 501 | TLWIRPLQSS APFTEDNNPT ITLSGPLTLL NEENRDPYDS IDLSEPLQNI |
| 551 | HLLSLSDVTA RHINTDNFHP ESLNATEHYG YQGIWSPYMV ETITTTNNAS |
| 601 | IETANTLYRA LYANWTPLGY KVNPEYQGDL ATTPLWQSPH TMFSLLRSYN |
| 651 | RTGDSDIERP FLEIQGIADG LFVHQNSIPG APGFRIQSTG YSLQASSETS |
| 701 | LHQKISLGFA QFFTRTKEIG SSNNVSAHNT VSSLYVELPW FQEAFATSTV |
| 751 | LAYGYGDHHL HSLHPSHQEQ AEGTCYSHTL AAAIGCSFPW QQKSYLHLSP |
| 801 | FVQAIAIRSH QTAFEEIGDN PRKFVSQKPF YNLTLPLGIQ GKWQSKFHVP |
| 851 | TEWTLELSYQ PVLYQQNPQI GVTLLASGGS WDILGHNYVR NALGYKVHNQ |
| 901 | TALFRSLDLF LDYQGSVSSS TSTHHLQAGS TLKF* |

The cp6752 nucleotide sequence <SEQ ID 8> is:

| | |
|---|---|
| 1 | ATGTTCGGGA TGACTCCTGC AGTGTATAGT TTACAAACGG ACTCCCTTGA |
| 51 | AAAGTTTGCT TTAGAGAGGG ATGAAGAGTT TCGTACGAGC TTTCCTCTCT |
| 101 | TAGACTCTCT CTCCACTCTT ACAGGATTTT CTCCAATAAC TACGTTTGTT |
| 151 | GGAAATAGAC ATAATTCCTC TCAAGACATT GTACTTTCTA ACTACAAGTC |
| 201 | TATTGATAAC ATCCTTCTTC TTTGGACATC GGCTGGGGGA GCTGTGTCCT |
| 251 | GTAATAATTT CTTATTATCA AATGTTGAAG ACCATGCCTT CTTCAGTAAA |
| 301 | AATCTCGCGA TTGGGACTGG AGGCGCGATT GCTTGCCAGG GAGCCTGCAC |
| 351 | AATCACGAAG AATAGAGGAC CCCTTATTTT TTTCAGCAAT CGAGGTCTTA |
| 401 | ACAATGCGAG TACAGGAGGA GAAACTCGTG GGGGTGCGAT TGCCTGTAAT |
| 451 | GGAGACTTCA CGATTTCTCA AAATCAAGGG ACTTTCTACT TTGTCAACAA |
| 501 | TTCCGTCAAC AACTGGGGAG GAGCCCTCTC CACCAATGGA CACTGCCGCA |
| 551 | TCCAAAGCAA CAGGGCACCT CTACTCTTTT TTAACAATAC AGCCCCTAGT |
| 601 | GGAGGGGGTG CGCTTCGTAG TGAAAATACA ACGATCTCTG ATAACACGCG |
| 651 | TCCTATTTAT TTTAAGAACA ACTGTGGGAA CAATGGCGGG GCCATTCAAA |
| 701 | CAAGCGTTAC TGTTGCGATA AAAAATAACT CCGGGTCGGT GATTTTCAAT |
| 751 | AACAACACAG CGTTATCTGG TTCGATAAAT TCAGGAAATG GTTCAGGAGG |
| 801 | GGCGATTTAT ACAACAAACC TATCCATAGA CGATAACCCT GGAACTATTC |
| 851 | TTTTCAATAA TAACTACTGC ATTCGCGATG GCGGAGCTAT CTGTACACAA |
| 901 | TTTTTGACAA TCAAAAATAG TGGCCACGTA TATTTCACCA ACAATCAAGG |
| 951 | AAACTGGGGA GGTGCTCTTA TGCTCCTACA GGACAGCACC TGCCTACTCT |
| 1001 | TCGCGGAACA AGGAAATATC GCATTTCAAA ATAATGAGGT TTTCCTCACC |
| 1051 | ACATTTGGTA GATACAACGC CATACATTGT ACACCAAATA GCAACTTACA |
| 1101 | ACTTGGAGCT AATAAGGGGT ATACGACTGC TTTTTTTGAT CCTATAGAAC |
| 1151 | ACCAACATCC AACTACAAAT CCTCTAATCT TTAATCCCAA TGCGAACCAT |
| 1201 | CAGGGAACGA TCTTATTTTC TTCAGCCTAT ATCCCAGAAG CTTCTGACTA |
| 1251 | CGAAAATAAT TTCATTAGCA GCTCGAAAAA TACCTCTGAA CTTCGCAATG |
| 1301 | GTGTCCTCTC TATCGAGGAT CGTGCGGGAT GGCAATTCTA TAAGTTCACT |
| 1351 | CAAAAAGGAG GTATCCTTAA ATTAGGGCAT GCGGCGAGTA TTGCAACAAC |
| 1401 | TGCCAACTCT GAGACTCCAT CAACTAGTGT AGGCTCCCAG GTCATCATTA |
| 1451 | ATAACCTTGC GATTAACCTC CCCTCGATCT TAGCAAAAGG AAAAGCTCCT |
| 1501 | ACCTTGTGGA TCCGTCCTCT ACAATCTAGT GCTCCTTTCA CAGAGGACAA |
| 1551 | TAACCCTACA ATTACTTTAT CAGGTCCTCT GACACTCTTA AATGAGGAAA |
| 1601 | ACCGCGATCC CTACGACAGT ATAGATCTCT CTGAGCCTTT ACAAAACATT |
| 1651 | CATCTTCTTT CTTTATCGGA TGTAACAGCA CGTCATATCA ATACCGATAA |
| 1701 | CTTTCATCCT GAAAGCTTAA ATGCGACTGA GCATTACGGT TATCAAGGCA |
| 1751 | TCTGGTCTCC TTATTGGGTA GAGACGATAA CAACAACAAA TAACGCTTCT |
| 1801 | ATAGAGACGG CAAACACCCT CTACAGAGCT CTGTATGCCA ATTGGACTCC |
| 1851 | CTTAGGATAT AAGGTCAATC CTGAATACCA AGGAGATCTT GCTACGACTC |
| 1901 | CCCTATGGCA ATCCTTTCAT ACTATGTTCT CTCTATTAAG AAGTTATAAT |
| 1951 | CGAACTGGTG ATTCTGATAT CGAGAGGCCT TTCTTAGAAA TTCAAGGGAT |
| 2001 | TGCCGACGGC CTCTTTGTTC ATCAAAATAG CATCCCCGGG GCTCCAGGAT |
| 2051 | TCCGTATCCA ATCTACAGGG TATTCCTTAC AAGCATCCTC CGAAACTTCT |
| 2101 | TTACATCAGA AAATCTCCTT AGGTTTTGCA CAGTTCTTCA CCCGCACTAA |
| 2151 | AGAAATCGGA TCAAGCAACA ACGTCTCGGC TCACAATACA GTCTCTTCAC |
| 2201 | TTTATGTTGA GCTTCCGTGG TTCCAAGAGG CCTTTGCAAC ATCCACAGTG |
| 2251 | TTAGCGTATG GCTATGGGGA CCATCACCTC CACAGCCTAC ATCCCTCACA |
| 2301 | TCAAGAACAG GCAGAAGGGA CGTGTTATAG CCATACATTA GCAGCAGCTA |
| 2351 | TCGGCTGTTC TTTCCCTTGG CAACAGAAAT CCTATCTTCA CCTCAGCCCG |
| 2401 | TTCGTTCAGG CAATTGCAAT ACGTTCTCAC CAAACAGCGT TCGAAGAGAT |
| 2451 | TGGTGACAAT CCCCGAAAGT TTGTCTCTCA AAAGCCTTTC TATAATCTGA |
| 2501 | CCTTACCTCT AGGAATCCAA GGAAAATGGC AGTCAAAATT CCACGTACCT |
| 2551 | ACAGAATGGA CTCTAGAACT TTCTTACCAA CCGGTACTCT ATCAACAAAA |
| 2601 | TCCCCAAATC GGTGTCACGC TACTTGCGAG CGGAGGTTCC TGGGATATCC |
| 2651 | TAGGCCATAA CTATGTTCGC AATGCTTTAG GGTACAAAGT CCACAATCAA |
| 2701 | ACTGCGCTCT TCCGTTCTCT CGATCTATTC TTGGATTACC AAGGATCGGT |
| 2751 | CTCCTCCTCG ACATCTACGC ACCATCTCCA AGCAGGAAGT ACCTTAAAAT |
| 2801 | TCTAA |

The PSORT algorithm predicts a cytoplasmic location (0.138).

The protein was expressed in E. coli and purified as a his-tag product, as shown in FIG. 4A, and also as a GST-fusion. The recombinant proteins were used to immunise mice, whose sera were used in a Western blot (4B) and the his-tagged protein was used for FACS analysis (4C).

The cp6752 protein was also identified in the 2D-PAGE experiment (Cpn0467).

These experiments show that cp6752 is a surface-exposed and immunoaccessible protein, and that it is a useful immunogen. These properties are not evident from the sequence alone.

### Example 5

The following C. pneumoniae protein (PID 4376850) was expressed <SEQ ID 9; cp6850>:

| | |
|---|---|
| 1 | MKKAVLIAAM FCGVVBLSBC CRIVDCCFBP PCAPSSCNPC |

EVIRKKERSC

| | |
|---|---|
| 51 | GGNACGSYVP SCSNPCGSTE CNSQSPQVKG CTSPDGRCKQ * |

A predicted signal peptide is highlighted.

The cp6850 nucleotide sequence <SEQ ID 10> is:

| | |
|---|---|
| 1 | ATGAAGAAAG CTGTTTTAAT TGCTGCAATG TTTTGTGGAG |

TAGTTAGCTT

| | |
|---|---|
| 51 | AAGTAGCTGC TGCCGCATTG TAGATTGTTG TTTTGAGGAT |

CCTTGCGCAC

| | |
|---|---|
| 101 | CCTCTTCTTG CAATCCTTGT GAAGTAATAA GAAAAAAAGA |

AAGATCTTGC

| | |
|---|---|
| 151 | GGCGGTAATG CTTGTGGGTC CTACGTTCCT TCTTGTTCTA |

ATCCATGTGG

| | |
|---|---|
| 201 | TTCAACAGAG TGTAACTCTC AAAGCCCACA AGTTAAAGGT |

TGTACATCAC

| | |
|---|---|
| 251 | CTGATGGCAG ATGCAAACAG TAA |

The PSORT algorithm predicts an inner membrane location (0.329).

The protein was expressed in E. coli and purified as a GST-fusion product, as shown in FIG. 5A. The recombinant protein was used to immunise mice, whose sera were used in a Western blot (FIG. 5B) and for FACS analysis (FIG. 5B). A his-tagged protein was also expressed.

These experiments show that cp6850 is a surface-exposed and immunoaccessible protein, and that it is a useful immunogen. These properties are not evident from the sequence alone.

### Example 6

The following C. pneumoniae protein (PID 4376900) was expressed <SEQ ID 11; cp6900>:

| | |
|---|---|
| 1 | MKIKFSWKVN FLICLLAVGL IFFGCSRVKR EVLVGRDATW |

FPKQFGIYTS

| | |
|---|---|
| 51 | DTNAFLNKLV SEINYKENLN INIVNQDWVH LFENLDDKKT |

QGAFTSVLPT

| | |
|---|---|
| 101 | LEMLEHYQFS DPILLTGPVL VVAQDSPYQS IEDLKGRLIG |

VYKFDSSVLV

| | |
|---|---|
| 151 | AQNIPDAVIS LYQHVPIALE ALTSNCYDAL LAPVIEVTAL |

IETAYKGRLL

| | |
|---|---|
| 201 | IISKPLNADG LRLAILKGTN GDLLEGFNAG LVKTRRSGKY |

DAIKQRYRLP

The cp6900 nucleotide sequence <SEQ ID 12> is:

| | |
|---|---|
| 1 | GTGAAGATAA AATTTTCTTG GAAGGTAAAT TTTTTAATAT |

GTTTACTGGC

| | |
|---|---|
| 51 | TGTGGGACTG ATCTTTTTCG GGTGCTCTCG AGTAAAAAGA |

GAAGTTCTCG

| | |
|---|---|
| 101 | TAGGTCGTGA TGCCACCTGG TTTCCAAAAC AATTCGGCAT |

TTATACATCC

| | |
|---|---|
| 151 | GATACCAACG CATTTTTAAA CGATCTTGTT TCTGAGATTA |

ACTATAAAGA

| | |
|---|---|
| 201 | GAATCTAAAT ATTAATATTG TAAATCAAGA TTGGGTGCAT |

CTCTTTGAGA

| | |
|---|---|
| 251 | ATTTAGATGA TAAAAAGACC CAAGGAGCAT TTACATCTGT |

ATTGCCTACT

| | |
|---|---|
| 301 | CTTGAGATGC TCGAACACTA TCAATTTTCT GATCCCATTT |

TACTCACAGG

| | |
|---|---|
| 351 | TCCTGTCCTT GTCGTCGCTC AAGACTCTCC TTACCAATCT |

ATAGAGGATC

| | |
|---|---|
| 401 | TTAAAGGTCG TCTTATTGGA GTGTATAAGT TTGACTCTTC |

AGTTCTTGTA

| | |
|---|---|
| 451 | GCTCAAAATA TCCCTGACGC TGTGATTAGC CTCTACCAAC |

ATGTTCCAAT

| | |
|---|---|
| 501 | AGCATTGGAA GCCTTAACAT CGAATTGTTA CGACGCTCTT |

CTAGCTCCTG

| | |
|---|---|
| 551 | TAATTGAAGT GACCGCGCTA ATAGAAACAG CATATAAAGG |

AAGACTGAAA

| | |
|---|---|
| 601 | ATTATTTCAA AACCCTTAAA CGCAGATGGT TTGCGGCTTG |

CAATACTGAA

| | |
|---|---|
| 651 | AGGGACAAAC GGAGATTTGC TTGAAGGGTT TAACGCAGGA |

CTTGTGAAAA

| | |
|---|---|
| 701 | CACGACGCTC AGGAAAATAC GATGCTATAA AACAGCGGTA |

TCGTCTTCCC

| | |
|---|---|
| 751 | TAA |

The PSORT algorithm predicts an inner membrane location (0.452).

The protein was expressed in E. coli and purified as a GST-fusion product, as shown in FIG. 6A. The recombinant protein was used to immunise mice, whose sera were used for FACS analysis (FIG. 6B). A his-tagged protein was also expressed.

The cp6900 protein was also identified in the 2D-PAGE experiment (Cpn0604).

These experiments show that cp6900 is a surface-exposed and immunoaccessible protein, and that it is a useful immunogen. These properties are not evident from the sequence alone.

### Example 7

The following C. pneumoniae protein (PID 4377033) was expressed <SEQ ID 13; cp7033>:

| | |
|---|---|
| 1 | MVNPIGPGPI DETERTPPAD LSAQGLEASA ANKSAEAQRI |

AGAEAKPKES

| | |
|---|---|
| 51 | KTDSVERWSI LRSAVNALMS LADKLGIASS NSSSSTSRSA |

DVOSTTATAP

| | |
|---|---|
| 101 | TPPPPTFDDY KTQAQTAYDT IFTSTSLADI QAALVSLQDA |

VTNIKDTAAT

| | |
|---|---|
| 151 | DEEWAIAAEW ETRNADAVKV GAQITELAKY ASDNQAILDS |

LGKLTSFDLL

| | |
|---|---|
| 201 | QAALLQSVAN NNKAAELLKE MQDNPVVPGK TPAIAQSLVD |

QTDATATQIE

| | |
|---|---|
| 251 | KDGNAIRDAY FAGQNASGAV EWAXSNNSIS NIDSAXAAIA |

TAKTQIAEAQ

| | |
|---|---|
| 301 | KRFPDSPILQ EAEQMVIQAE KDLKNIKPAD GSDVPNPGTT |

VGGSKQQGSS

| | |
|---|---|
| 351 | IGSIRVSMLL DDAENETASI LMSGFRQMIH MFNTENPDSQ |

AAQQELAAQA

| | |
|---|---|
| 401 | RAARAAGDPS AAAALADAQK ALEAALGKAG QQQGILNALG |

QIASAAVVSA

| | |
|---|---|
| 451 | GVPPAAASSI GSSVKQLYKT SKBTGSDYKT QISAGYDAYK |

SINDAYGRAR

| | |
|---|---|
| 501 | NDATRDVINN VSTPALTRSV PRARTEARGP EKTDQALARV |

ESGNSRTLGD

| | |
|---|---|
| 551 | VYSQVSALQS VMQIIQSNPQ ANNEEIRQKL TSAVTKPPQF |

GYPYVQLSND

| | |
|---|---|
| 601 | STQKFIAKLE SLFAEGSRTA AEIKALSFET NSLFIQQVLV |

NIGSLYSGYL

| | |
|---|---|
| 651 | Q* |

The cp7033 nucleotide sequence <SEQ ID 14> is:

| | |
|---|---|
| 1 | ATGGTTAATC CTATTGGTCC AGGTCCTATA GACGAAACAG AACGCACACC |
| 51 | TCCCGCAGAT CTTTCTGCTC AAGGATTGGA GGCGAGTGCA GCAAATAAGA |
| 101 | GTGCGGAAGC TCAAAGAATA GCAGGTGCGG AAGCTAAGCC TAAAGAATCT |
| 151 | AAGACCGATT CTGTAGAGCG ATGGAGCATC TTGCGTTCTG CAGTGAATGC |
| 201 | TCTCATGAGT CTGGCAGATA AGCTGGGTAT TGCTTCTAGT AACAGCTCGT |
| 251 | CTTCTACTAG CAGATCTGCA GACGTGGACT CAACGACAGC GACCGCACCT |
| 301 | ACGCCTCCTC CACCCACGTT TGATGATTAT AAGACTCAAG CGCAAACAGC |
| 351 | TTACGATACT ATCTTTACCT CAACATCACT AGCTGACATA CAGGCTGCTT |
| 401 | TGGTGAGCCT CCAGGATGCT GTCACTAATA TAAAGGATAC AGCGGCTACT |
| 451 | GATGAGGAAA CCGCAATCGC TGCGGAGTGG GAAACTAAGA ATGCCGATGC |
| 501 | AGTTAAAGTT GGCCCGCAAA TTACAGAATT AGCGAAATAT GCTTCGGATA |
| 551 | ACCAAGCGAT TCTTGACTCT TTAGGTAAAC TGACTTCCTT CGACCTCTTA |
| 601 | CAGGCTGCTC TTCTCCAATC TGTAGCAAAC AATAACAAAG CAGCTGAGCT |
| 651 | TCTTAAAGAG ATGCAAGATA ACCCAGTAGT CCCAGGGAAA ACGCCTGCAA |
| 701 | TTGCTCAATC TTTAGTTGAT CAGACAGATG CTACAGCGAC ACAGATAGAG |
| 751 | AAAGATGGAA ATGCGATTAG GGATGCATAT TTTGCAGGAC AGAACGCTAG |
| 801 | TGGAGCTGTA GAAAATGCTA AATCTAATAA CAGTATAAGC AACATAGATT |
| 851 | CAGCTAAAGC AGCAATCGCT ACTGCTAAGA CACAAATAGC TGAAGCTCAG |
| 901 | AAAAAGTTCC CCGACTCTCC AATTCTTCAA GAAGCGGAAC AAATGGTAAT |
| 951 | ACAGGCTGAG AAAGATCTTA AAAATATCAA ACCTGCAGAT GGTTCTGATG |
| 1001 | TTCCAAATCC AGGAACTACA GTTGGAGGCT CCAAGCAACA AGGAAGTAGT |
| 1051 | ATTGGTAGTA TTCGTGTTTC CATGCTGTTA GATGATGCTG AAAATGAGAC |
| 1101 | CGCTTCCATT TTGATGTCTG GGTTTCGTCA GATGATTCAC ATGTTCAATA |
| 1151 | CGGAAAATCC TGATTCTCAA GCTGCCCAAC AGGAGCTCGC AGCACAAGCT |
| 1201 | AGAGCAGCGA AAGCCGCTGG AGATOACACT GCTGCTGCAG CGCTGGCAGA |
| 1251 | TGCTCAQAAA GCTTTAGAAG CGGCTCTAGG TAAAGCTGGG CAACAACAGG |
| 1301 | GCATACTCAA TGCTTTAGGA CAGATCGCTT CTGCTGCTGT TGTGAGCGCA |
| 1351 | GGAGTTCCTC CCGCTGCAGC AAGTTCTATA GGGTCATCTG TAAAACAGCT |
| 1401 | TTACAAGACC TCAAAATCTA CAGGTTCTGA TTATAAAACA CAGATATCAG |
| 1451 | CAGGTTATGA TGCTTACAAA TCCATCAATG ATGCCTATGG TAGGGCACGA |
| 1501 | AATGATGCGA CTCGTGATGT GATAAACAAT GTAAGTACCC CCGCTCTCAC |
| 1551 | ACGATCCGTT CCTAGAGCAC GAACAGAAGC TCGAGGACCA GAAAAAACAG |
| 1601 | ATCAAGCCCT CGCTAGGGTG ATTTCTGGCA ATAGCAGAAC TCTTCGAGAT |
| 1651 | GTCTATAGTC AAGTTTCGGC ACTACAATCT GTAATGCAGA TCATCCAGTC |
| 1701 | GAATCCTCAA GCGAATAATG AGGAGATCAG ACAAAAGCTT ACATCGGCAG |
| 1751 | TGACAAAGCC TCCACAGTTT GGCTATCCTT ATGTGCAACT TTCTAATGAC |
| 1801 | TCTACACAGA AGTTCATAGC TAAATTAGAA AGTTTGTTTG CTGAAGGATC |
| 1851 | TAGGACAGCA GCTGAAATAA AAGCACTTTC CTTTGAAACG AACTCCTTGT |
| 1901 | TTATTCAGCA GGTGCTGGTC AATATCGGCT CTCTATATTC TGGTTATCTC |
| 1951 | CAATAA |

The PSORT algorithm predicts a cytoplasmic location (0.272).

The protein was expressed in E. coli and purified as a GST-fusion product, as shown in FIG. 7A. A his-tagged protein was also expressed. The recombinant proteins were used to immunise mice, whose sera were used for FACS (FIG. 7B) and Western blot (7C) analyses.

The cp7033 protein was also identified in the 2D-PAGE experiment (Cpn0728) and showed good cross-reactivity with human sera, including sera from patients with pneumonitis.

These experiments show that cp7033 a surface-exposed and immunoaccessible protein, and that it is a useful immunogen. These properties are not evident from the sequence alone.

### Example 8

The following C. pneumoniae protein (PID 6172321) was expressed <SEQ ID 15; cp0017>:

| | |
|---|---|
| 1 | MGIKGTGIIV WVDDATAKTK NATLTWTKTG YKPNPERQGP |

LVPNSLWGSF

| | |
|---|---|
| 51 | VDVRSIQSLM DRSTSSLSSS TNLWVSGIAD FLHEDQKGNQ |

RSYRHSSAGY

| | |
|---|---|
| 101 | ALGGGFFTAS ENFFNPAFCQ LFGYDKDHLV AKNHTHVYAQ |

AMSYRHLGES

| | |
|---|---|
| 151 | KTLAKILSGN SDSLPFVFNA RFAYGHTDNN MTTKYTGYSP |

VKGSWGNDAF

| | |
|---|---|
| 201 | GIECGGAIPV VASGRRSWVD THTPFLNLEM IYAHQNDFKE |

NGTEGRSFQS

| | |
|---|---|
| 251 | EDLFNLAVPV GIKPEEFSDK STYDLSIAYV PDVIRNDPGC |

TTTLMVSGDS

| | |
|---|---|
| 301 | WSTCGTSLSR QALLVRAGNH HAFASNFEVF SQFEVELRGS |

SRSYAIDLGG

| | |
|---|---|
| 351 | RFGF* |

The cp0017 nucleotide sequence <SEQ ID 16> is:

| | |
|---|---|
| 1 | ATGGGTATCA AGGGAACTGG AATAATTGTT TGGGTCGACG |

ATGCAACTGC

| | |
|---|---|
| 51 | AAAAACAAAA AATGCTACCT TAACTTGGAC TAAAACAGGA |

TACAAGCCGA

| | |
|---|---|
| 101 | ATCCAGAACG TCAGGGACCT TTGGTTCCTA ATAGCCTGTG |

GGGTTCTTTT

| | |
|---|---|
| 151 | GTCGATGTCC GCTCCATTCA GAGCCTCATG GACCGGAGCA |

CAAGTFCGTT

| | |
|---|---|
| 201 | ATCTTCGTCA ACAAATTTGT GGGTATCAGG AATCGCGGAC |

TTTTTGCATQ

| | |
|---|---|
| 251 | AAGATCAGAA AGGAAACCTT CGTAGTTATC GTCATTCTAG |

CGCGGGTTAT

| | |
|---|---|
| 301 | GCATTAGGAG GAGGATTCTT CACGGCTTCT GAAAATTTCT |

TTAATTTTGC

| | |
|---|---|
| 351 | TTTTTGTCAG CTTTTTGGCT ACGACAAGGA CCATCTTGTG |

GCTAAGAACC

| | |
|---|---|
| 401 | ATACCCATGT ATATGCAGGG GCAATGAGTT ACCQACACCT |

CGGAGAGTCT

| | |
|---|---|
| 451 | AAGACCCTCC CTAAGATTTT GTCAGGAAAT TCTGACTCCC |

TACCTTTTGT

| | |
|---|---|
| 501 | CTTCAATGCT CGGTTTGCTT ATGGCCATAC CGACAATACT |

ATGACCACAA

| | |
|---|---|
| 551 | AGTACACTGG CTATTCTCCT GTTAAGGGAA GCTGGGGAAA |

TGATGCCTTC

| | |
|---|---|
| 601 | GGTATAGAAT GTCGAGGAGC TATCCCGGTA GTTGCTTCAG |

GACGTCGGTC

| | |
|---|---|
| 651 | TTGGGTGGAT ACCCACACGC CATTTCTAAA CCTAGAGATG |

ATCTATGCAC

| | |
|---|---|
| 701 | ATCAGAATGA CTTTAAGGAA AACGGCACAG AAGGCCGTTC |

TTTCCAAAGT

| | |
|---|---|
| 751 | GAAGACCTCT TCAATCTAGC GGTTCCTGTA GGGATAAAAT |

TTGAGAAATT

| | |
|---|---|
| 801 | CTCCGATAAG TCTACGTATG ATCTCTCCAT AGCTTACGTT |

CCCGATGTGA

| | |
|---|---|
| 851 | TTCGTAATGA TCCAGGCTGC ACGACAACTC TTATGGTTTC |

TGGGGATTCT

| | |
|---|---|
| 901 | TGGTCGACAT GTGGTACAAG CTTGTCTAGA CAAGCTCTTC |

TTGTACGTGC

| | |
|---|---|
| 951 | TGGAAATCAT CATGCCTTTG CTTCAAACTT TGAAGTTTTC |

AGTCAGTTTG

| | |
|---|---|
| 1001 | AAGTCGAGTT GCGAGGTTCT TCTCGTAGCT ATGCTATCGA |

TCTTGGAGGA

| | |
|---|---|
| 1051 | AGATTCGGAT TTTAA |

This sequence is frame-shifted with respect to cp0016.

The PSORT algorithm predicts a cytoplasmic location (0.075).

The protein was expressed in E. coli and purified as a GST-fusion product, as shown in FIG. 8A. The recombinant protein was used to immunise mice, whose sera were used in a Western blot (FIG. 8B) and for FACS analysis (FIG. 8C). A his-tagged protein was also expressed.

This protein also showed good cross-reactivity with human sera, including sera from patients with pneumonitis.

These experiments show that cp0017 is a surface-exposed and immunoaccessible protein, and that it is a useful immunogen. These properties are not evident from the sequence alone.

### Example 9

The following C. pneumoniae protein (PID 6172315) was expressed <SEQ ID 17; cp0014>:

| | |
|---|---|
| 1 | MKSSFPKFVF STFAIFPLSM IATETVLDSS ASFDGNKNGN |

FSVRESQEDA

| | |
|---|---|
| 51 | GTTYLFKGNV TLENIFGTGT AITKSCFNNT KGDLTFTGNG |

NSLLFQTVDA

| | |
|---|---|
| 101 | GTVAGAAVNS SVVDKSTTFI GFSSLSFIAS PGSSITTGKG |

AVSCSTGSLS

| | |
|---|---|
| 151 | LTKMSVCSSA KTFQRIMAVL SPQKLFH* |

The cp0014 nucleotide sequence <SEQ ID 18> is:

| | |
|---|---|
| 1 | ATGAAGTCTT CTPTCCCCAA GTTTGTATTT TCTACATTTG |

CTATTTTCCC

| | |
|---|---|
| 51 | TTTGTCTATG ATTGCTACCG AGACAGTTTT GGATTCAAGT |

GCGAGTTUCG

| | |
|---|---|
| 101 | ATGGGAATAA AAATGGTAAT TTTTCAGTTC GTGAGAGTCA |

GGAAGATGCT

| | |
|---|---|
| 151 | GGAACTACCT ACCTATTTAA GGGAAATGTC ACTCTAGAAA |

ATATTCCTGG

| | |
|---|---|
| 201 | AACAGGCACA GCAATCACAA AAAGCTGTTT TAACAACACT |

AAGGGCGATT

| | |
|---|---|
| 251 | TGACTTTCAC AGGTAACGGG AACTCTCTAT TGTTCCAAAC |

GGTGGATGCA

| | |
|---|---|
| 301 | GGGACTGTAG CAGGGGCTGC TGTTAACAGC AGCGTGGTAG |

ATAAATCTAC

| | |
|---|---|
| 351 | CACGTTTATA GGGTTTTCTT CGCTATCTTT TATTGCGTCT |

CCTGGAAGTT

| | |
|---|---|
| 401 | CGATAACTAC CGGCAAAGGA GCCGTTAGCT GCTCTACGGG |

TAGCTTGAGT

| | |
|---|---|
| 451 | TTGACAAAAA TGTCAGTTTG CTCTTCAGCA AAAACTTTTC |

AACGGATAAT

| | |
|---|---|
| 501 | GGCGGTGCTA TCACCGCAAA AACTCTTTCA TTAA |

This protein is frame-shifted with respect to cp0015.

The PSORT algorithm predicts an inner membrane location (0.047). The protein was expressed in E. coli and purified as a his-tag product, as shown in FIG. 9A. A GST-fusion was also expressed. The recombinant proteins were used to immunise mice, whose sera were used in an immunoassay (FIG. 9B) and for FACS analysis (FIG. 9C).

This protein also showed good cross-reactivity with human sera, including sera from patients with pneumonitis.

These experiments suggest that cp0014 is a useful immunogen. These properties are not evident from the sequence alone.

### Example 10

The following C. pneumoniae protein (PID 6172317) was expressed <SEQ ID 19; cp0015>:

| | |
|---|---|
| 1 | MSALFSENTS SKKGGAIQTS DALTITGNQG EVSFSDNTSS DSGAAIFTEA |
| 51 | SVTISNNAKV SFIDNKVTGA SSSTTGDMSG GAICAYKTST DTKVTLTGNQ |
| 101 | NLLFSNNTST TAGGAIYVKK LBLASGGLTL FSRNSVNGGT APKGGAIAIE |
| 151 | DSGELSLSAD SGDIVFLGNT VTSTTPGTNR SSIDLGTSAK MTALRSAAGR |
| 201 | AIYFYDPITT GSSTTVTDVL KVNETPADSA LQYTGNIIPT GEKLSETEAA |
| 251 | DSKNLTSKLL QPVTLSGGTL SLKHGVTLQT QAFTQQADSR LEMDVGTTLE |
| 301 | PADTSTINNL VINISSIDGA KKAKIETKAT SKNLTLSGTI TLLDPTGTFY |
| 351 | ENHSLRNPQS YDILELKASG TVTSTAVTPD PIMGEKFHYG YQGTWGPIVW |
| 401 | GTGASTTATF NWTKTGYIPN PERIGSLVPN SLWNAFIDIS SLHYLMETAN |
| 451 | EGLQGDRAFW CAGLSNFFHK DSTITRRGFR HLSGGYVIGG NLHTCSDKIL |
| 501 | SAAFCQLFGR DRDYFVAKWQ GTVYGGTLYY QHNETYISLP CRLRPCSLSY |
| 551 | VPTEIPVLFS GNLSYTHTDN DLKTKYTTYP TVKGSWGNDS PALEPGGRAP |
| 601 | ICLDESALFE QYNPFMKLQF VYAHQEGFKE QGTEAREFGS SRLVNLALPI |
| 651 | GIRFDKESDC QDATYNLTLG YTVDLVRSNP DCTTTLRISG DSWKTFGTNL |
| 701 | ARQALVLRAG NHFCFNSNFE AFSQFSFELR GSSRNYNVDL GAKYQF* |

This sequence is frame-shifted with respect to cp0014.

The cp0015 nucleotide sequence <SEQ ID 20> is:

| | |
|---|---|
| 1 | ATCTCAGCTC TGTTTTCTGA AAATACCTCC TCAAAGAAAG GCGGAGCCAT |
| 51 | TCAGACTTCC GATGCCCTTA CCATTACTGG AAACCAAGGG GAAGTCTCTT |
| 101 | TTTCTGACAA TACTTCTTCG GATTCTGGAG CTGCAATTTT TACAGAAGCC |
| 151 | TCGGTGACTA TTTTCTAAAA TGCTAAAGTT TCCTTTATTG ACAATAAGGT |
| 201 | CACAGGAGCG AGCTCCTCAA CAACGGGGGA TATGTGAGGA GGTGCTATCT |
| 251 | GTGCTTATAA AACTAGTACA GATACTAAGG TCACCCTCAC TGGAAATCAG |
| 301 | ATGTTACTCT TCAGCAACAA TACATCGACA ACAGCGGGAG GAGCTATCTA |
| 351 | TGTGAAAAAG CTCGAACTGG CTTCCGGAGG ACTTACCCTA TTCAGTAGAA |
| 401 | ATAGTGTCAA TGGAGGTACA GCTCCTAAAG GTGGAGCCAT AGCTATCGAA |
| 451 | GATAGTGGGG AATTGAGTTT ATCCGCCGAT AGTGGTGACA TTGTCTTTTT |
| 501 | AGGGAATACA GTCACTTCTA CTACTCCTGG GACGAATAGA AGTAGTATCG |
| 551 | ACTTAGGAAC GAGTGCAAAG ATGACAGCTT TGCGTTCTGC TGCTGGTAGA |
| 601 | GCCATCTACT TCTATGATCC CATAACTACA GGATCATCCA CAACAGTTAC |
| 651 | AGATGTCTTA AAAGTTAATG AGACTCCGGC AGATTCTGCA CTACAATATA |
| 701 | CAGGGAACAT CATCTTCACA GGAGAAAAGT TATCAGAGAC AGAGGCCGCA |
| 751 | GATTCTAAAA ATCTTACTTC GAAGCTACTA CAGCCTGTAA CTCTTTCAGG |
| 801 | AGGTACTCTA TCTTTAAAAC ATGGAGTGAC TCTGCAGACT CAGGCATTCA |
| 851 | CTCAACAGGC AGATTCTCGT CTCGAAATGG ACGTAGGAAC TACTCTAGAA |
| 901 | CCTGCTGATA CTAGCACCAT AAACAATTTG GTCATTAACA TCAGTTCTAT |
| 951 | AGACGGTGCA AAGAAGGCAA AAATAGAAAC CAAAGCTACG TCAAAAAATC |
| 1001 | TGACTTTATC TGGAACCATC ACTTTATTGG ACCCGACGGG CACGTTTTAT |
| 1051 | GAAAATCATA GTTTAAGAAA TCCTCAGTCC TACGACATCT TAGAGCTCAA |
| 1101 | AGCTTCTGGA ACTGTAACAA GCACCGCAGT GACTCCAGAT CCTATAATGG |
| 1151 | GTGAGAAATT CCATTACGGC TATCAGGGAA CTTGGGGCCC AATTGTTTGG |
| 1201 | GGGACAGGGG CTTCTACGAC TGCAACCTTC AACTGGACTA AAACTGGCTA |
| 1251 | TATTCCTAAT CCCGAGCGTA TCGGCTCTTT AGTCCCTAAT AGCTTATGGA |
| 1301 | ATGCATTTAT AGATATTAGC TCGCTCCATT ATCTTATGGA GACTGCAAAC |
| 1351 | GAAGGGTTGC AGGGAGACCG TGCTTTTTGG TGTGCTGGAT TATCTAACTT |
| 1401 | CTTCCATAAG GATAGTACAA AAACACGACG CGGGTTTCGC CATTTGAGTG |
| 1451 | GCGGTTATGT CATAGGAGGA AACCTACATA CTTGTTCAGA TAAGATTCTT |
| 1501 | AGTGCTGCAT TTTGTCAGCT CTTTGGAAGA GATAGAGACT ACTTTGTAGC |
| 1551 | TAAGAATCAA GGTACAGTCT ACGGAGGAAC TCTCTATTAC CAGCACAACG |
| 1601 | AAACCTATAT CTCTCTTCCT TGCAAACTAC GGCCTTGTTC GTTGTCTTAT |
| 1651 | GTTCCTACAG AGATTCCTGT TCTCTTTTCA GGAAACCTTA GCTACACCCA |
| 1701 | TACGGATAAC GATCTGAAAA CCAAGTATAC AACATATCCT ACTGTTAAAG |
| 1751 | GAAGCTGGGG GAATGATAGT TTCGCTTTAG AATTCGGTGG AAGAGCTCCG |
| 1801 | ATTTGCTTAG ATGAAAGTGC TCTATTTGAG CAGTACATGC CCTTCATGAA |
| 1851 | ATTGCAGTTT GTCTATGCAC ATCAGGAAGG TTTTAAAGAA CAGGGAACAG |
| 1901 | AAGCTCGTGA ATTTGGAAGT AGCCGTCTTG TGAATCTTGC CTTACCTATC |
| 1951 | GGGATCCGAT TTGATAAGGA ATCAGACTGC CAAGATGCAA CGTACAATCT |
| 2001 | AACTCTTGGT TATACTGTGG ATCTTGTTCG TAGTAACCCC GACTGTACGA |
| 2051 | CAACACTGCG AATTAGCGGT GATTCTTGGA AAACCTTCGG TACGAATTTG |
| 2101 | GCAAGACAAG CTTTAGTCCT TCGTGCAGGG AACCATTTTT GCTTTAACTC |
| 2151 | AAATTTTGAA GCCTTTAGCC AATTTTCTTT TGAATTGCGT GGGTCATCTC |
| 2201 | GCAATTACAA TGTAGACTTA GGAGCAAAAT ACCAATTCTA A |

The PSORT algorithm predicts a cytoplasmic location (0.274).

The protein was expressed in E. coli and purified as a GST-fusion product, as shown in FIG. 10A. The recombinant protein was used to immunise mice, whose sera were used in a Western blot (FIG. 10B) and for FACS analysis. A his-tagged protein was also expressed.

These experiments show that cp0015 is a useful immunogen. These properties are not evident from the sequence alone.

### Example 11

The following C. pneumoniae protein (PID 6172325) was expressed <SEQ ID 21; cp0019>:

| | |
|---|---|
| 1 | LQDSQDYSFV KLSPGAGGTI ITQDASQKPL EVAPSRPHYG YQGHWNVQVI |
| 51 | PGTGTQPSQA NLEWVRTGYL PNPERQGSLV PNSLWGSFVD QRAIQEINVN |
| 101 | SSQILCQERG VWGAGIANFL HRDKINEHGY RHSGVGYLVG VGTHAFSDAP |
| 151 | INAAPCQLFS RDKDYVVSKN HGTSYSGVVF LEDTLEFRSP QGPYTDSSSE |
| 201 | ACCNQVVTID MQLSYSHRNN DMKTKYTTYP EAQGSWANDV FGLEFGATTY |
| 251 | YYPNSTFLFD YYSPFLRLQC TYAHQEDFKE TGGEVRHFTS GDLFNLAVPI |
| 301 | GVKFERPSDC KRGSYELTLA YVPDVIRXDP KSTATLASGA TWSTHGNNLS |
| 351 | RQGLQLRLGN HCLINPGIEV FSHGAIEELG SSRNYNINLG GKYRF* |

This sequence is frame-shifted with respect to cp0018.

The cp0019 nucleotide sequence <SEQ ID 22> is:

| | |
|---|---|
| 1 | TTGCAAGACT CTCAAGACTA TAGCTTTGTA AAGTTATCTC CAGGAGCGGG |
| 51 | AGGGACTATA ATTACTCAAG ATGCTTCTCA GAAGCCTCTT GAAGTAGCTC |
| 101 | CTTCTAGACC ACATTATGGC TATCAAGGAC ATTGGAATGT GCAAGTCATC |
| 151 | CCAGGAACGG GAACTCAACC GAGCCAGGCA AATTTAGAAT GGGTGCGCAC |
| 201 | AGGATACCTT CCGAATCCCG AACGGCAAGG ATCTTTAGTT CCCAATAGCC |
| 251 | TGTGGGGTTC TTTTGTTGAT CAGCGTGCTA TCCAAGAAAT CATGGTAAAT |
| 301 | AGTAGCCAAA TCTTATGTCA GGAACGGGGA GTCTGGGGAG CTGGAATTGC |
| 351 | TAATTTCCTA CATAGAGATA AAATTAATGA GCACGGCTAT CGCCATAGCG |
| 401 | GTGTCGGTTA TCTTGTGGGA GTTGGCACTC ATGCTTTTTC TGATGCTACG |
| 451 | ATAAATGCGG CTTTTTGCCA GCTCTTCAGT AGAGATAAAG ACTACGTAGT |
| 501 | ATCCAAAAAT CATGGAACTA GCTACTCAGG GGTCGTATTT CTTGAGGATA |
| 551 | CCCTAGAGTT TAGAAGTCCA CAGGGATTCT ATACTGATAG CTCCTCAGAA |
| 601 | GCTTGCTGTA ACCAAGTCGT CACTATAGAT ATGCAGTTGT CTTACAGCCA |
| 651 | TAGAAATAAT GATATGAAAA CCAAATACAC GACATATCCA GAAGCTCAGG |
| 701 | GATCTTGGGC AAATGATGTT TTTGGTCTTG AGTTTGGAGC GACTACATAC |
| 751 | TACTACCCTA ACAGTACTTT TTTATTTGAT TACTACTCTC CGTTTCTCAG |
| 801 | GCTGCAGTGC ACCTATGCTC ACCAGGAAGA CTTCAAAGAG ACAGGAGGTG |
| 851 | AGGTTCGTCA CTTTACTAGC GGAGATCTTT TCAATTTAGC AGTTCCTATT |
| 901 | GGCGTGAAGT TTGAGAGATT TTCAGACTGT AAAAGGGGAT CTTATGAACT |
| 951 | TACCCTTGCT TATGTTCCTG ATGTGATTCG CAAAGATCCC AAGAGCACGG |
| 1001 | CAACATTGGC TAGTGGAGCT ACGTGGAGCA CCCACGGAAA CAATCTCTCC |
| 1051 | AGACAAGGAT TACAACTGCG TTTAGGGAAC CACTGTCTCA TAAATCCTGG |
| 1101 | AATTGAGGTG TTCAGTCACG GAGCTATTGA ATTGCGGGGA TCCTCTCGTA |
| 1151 | ATTATAACAT CAATCTCGGG GGTAAATACC GATTTTAA |

The PSORT algorithm predicts a cytoplasmic location (0.189).

The protein was expressed in E. coli and purified as a GST-fusion product, as shown in FIG. 11A. This protein was used to immunise mice, whose sera were used in a Western blot (FIG. 11B) and an immunoblot assay (FIG. 11C). A his-tagged protein was also expressed.

These experiments show that cp0019 is a useful immunogen. These properties are not evident from the sequence alone.

### Example 12

The following C. pneumoniae protein (PID 4376466) was expressed <SEQ ID 23; cp6466>:

| | |
|---|---|
| 1 | MRKISVGXCI TILLSLSVVL QGCKESSHSS TSRGELAINI RDEPRSLDPR |
| 51 | QVRLLSEISL VKRIYEGLVQ ENNLSGNIEP ALAEDYSLSS DGLTYTFKLK |
| 101 | SAPWSNGDPL TAEDFIBSWK QVATQEVSGI YAPALNPIKN VRXIQEGHLS |
| 151 | IDHFGVHSPN ESTLVVTLES PTSHPLKLLA LPVFFPVHKS QRTLQSKSLP |
| 201 | IASGAPYPKN IKQKQWIKLS KNPHYYNQSQ VETKTITIHF IPDANTAAKL |
| 251 | FNQGKLNWQG PPWGERIPQE TLSNLQSKGH LHSFDVAGTS WLTFNINKFP |
| 301 | LNNMKLRBAL ASALDKEALV STIFLGRAXT ADHLLPTNIH SYPEUQKQEH |
| 351 | AQRQAYAKKL PKEALEELQI TAKDLEHLNL IFPVSSSASB LLVQLIREQW |
| 401 | KESLGFAIPI VGKEFALLQA DLSSGNFSLA TGGWFADDAD PNAFLTIPAY |
| 451 | PSGVPPYAIN HKDFLEILQN IEQEQDHQKR SELVSQASLY LETFHIIEPI |
| 501 | YHDAFQFAMN KKLSNLGVSP TGVVDFRYAK EN* |

A predicted signal peptide is highlighted.

The cp6466 nucleotide sequence <SEQ ID 24> is:

| | |
|---|---|
| 1 | ATGCGCAAGA TATCAGTGGG AATCTGTATC ACCATTCTCC TTAGCCTCTC |
| 51 | CGTAGTCCTC CAAGGCTGCA AGGAGTCCAG TCACTCCTCT ACATCTCGGG |
| 101 | GAGAACTCGC TATTAATATA AGAGATGAAC CCCGTTCTTT AGATCCAAGA |
| 151 | CAAGTGCGAC TTCTTTCAGA AATCAGCCTT GTCAAACATA TCTATGAGGG |
| 201 | ATTAGTTCAA GAAAATAATC TTTQAGGAAA TATAGAGCCT GCTCTTGCAG |
| 251 | AAGACTACTC TCTTTCCTCG GACGGACTCA CTTATACTTT TAAACTGAAA |
| 301 | TCAGCTTTTT GGAGTAATGG CGACCCCTTA ACAGCTGAAG ACTTTATAGA |
| 351 | ATCTTGGAAA CAAGTAGCTA CTCAAGAAGT CTCAGGAATC TATGCTTTTG |
| 401 | CCTTGAATCC AATTAAAAAT GTACGAAAGA TCCAAGAGGG ACACCTCTCC |
| 451 | ATAGACCATT TTGGAGTGCA CTCTCCTAAT GAATCTACAC TTGTTGTTAC |
| 501 | CCTGGAATCC CCAACCTCGC ATTTCTTAAA ACTTTTAGCT CTTCCAGTCT |
| 551 | TTTTCCCCGT TCATAAATCT CAAAGAACCC TGCAATCCAA ATCTCTACCT |
| 601 | ATAGCAAGCG GAGCTTTCTA TCCTAAAAAT ATCAAACAAA AACAATGGAT |
| 651 | AAAACTCTCA AAAAACCCTC ACTACTATAA TCAAAGTCAG GTGGAAACTA |
| 701 | AAACGATTAC GATTCACTTC ATTCCCGATG CAAACACAGC AGCAAAACTA |
| 751 | TTTAATCAGG GAAAACTCAA TTGGCAAGGA CCTCCTTGGG GAGAACGCAT |
| 801 | TCCTCAAGAA ACCCTATCCA ATTTACAGTC TAAGGGGCAC TTACACTCTT |
| 851 | TTGATGTCGC AGGAACCTCA TGGCTCACCT TCAATATCAA TAAATTCCCC |
| 901 | CTCAACAATA TGAAGCTTAG AGAAGCCTTA GCATCAGCCT TAGATAAGGA |
| 951 | AGCTCTTGTC TCAACTATAT TCTTAGGCCG TGCAAAAACT GCCGATCATC |
| 1001 | TCCTACCTAC AAATATTCAT AGCTATCCCG AACATCAAAA ACAAGAGATG |
| 1051 | GCACAACGCC AAGCTTACGC TAAAAAACTC TTTAAAGAAG CTTTAGAAGA |
| 1101 | ACTCCAAATC ACTGCTAAAG ATCTCGAACA TCTTAATCTT ATCTTTCCCG |
| 1151 | TTTCCTCGTC AGCAAGTTCT TTACTAGTCC AACTTATACG AGAACAGTGG |
| 1201 | AAAGAAAGTT TAGGGTTCGC TATCCCTATT GTCGGAAAGG AATTTGCTCT |
| 1251 | TCTCCAAGCA GACCTATCTT CAGGGAACTT CTCTTTAGCT ACAGGAGGAT |
| 1301 | GGTTCGCAGA CTTTGCTGAT CCTATGGCAT TTCTAACGAT CTTTGCTTAT |
| 1351 | CCATCAGGAG TTCCTCCTTA TGCAATCAAC CATAAGGACT TCCTAGAAAT |
| 1401 | TCTACAAAAC ATAGAACAAG AGCAAGATCA CCAAAAACGC TCGGAATTAG |
| 1451 | TGTCGCAAGC TTCTCTTTAC CTAGAGACCT TTCATATTAT TGAGCCGATC |
| 1501 | TACCACGACG CATTTCAATT TGCTATGAAT AAAAAACTTT CTAATCTAGG |
| 1551 | AGTCTCACCA ACAGGAGTTG TGGACTTCCG TTATGCTAAG GAAAATTAG |

The PSORT algorithm predicts that the protein is an outer membrane lipoprotein (0.790).

The protein was expressed in E. coli and purified both as a GST-fusion product and a His-tag fusion product. Purification of the protein as a GST-fusion product is shown in FIG. 12A. The recombinant proteins were used to immunise mice, whose sera were used in Western blots (FIGS. 12B and 12C). FACS analysis was also performed.

These experiments show that cp6466 is a useful immunogen. These properties are not evident from the sequence alone.

### Example 13

The following C. pneumoniae protein (PID 4376468) was expressed <SEQ ID 25; cp6468>:

| | |
|---|---|
| 1 | MFSRWITLFL LFISLTGCSS YSSKHKQSLI IPIHDDPVAF SPEQAKRAND |
| 51 | LSIAQLLFDG LTRETHRESN DLELAIASRY TVSEDFCSYT FFIKDSALWS |
| 101 | DGTPITSEDI RNAWEYAQEN SPHIQIFQGL TWSTPSSNAI TIHLDSPNPD |
| 151 | FPKLLAPPAF AIFKPENPKL FSGPYTLVEY FPGHNIHLKK NPNYYDYHCV |
| 201 | SINSIKLLII PDIYTAIHLL NRGKVDWVGQ PWHQGIPWEL HKQSQYHYYT |
| 251 | YPVEGAPWLC LNTKSPHLND LQNRHRLATC IDKRSIIEEA LQGTQQPAET |
| 301 | LSRGAPQPNQ YKKQKPLTPQ EKLVLTYPSD ILRCQRIAEI LKEQWKAAGI |
| 351 | DLILEGLEYH LFVVNKRKQD YAIATQTGVA YYPGANLISE EDKLLQNFEI |
| 401 | IPIYYLSYDY LTQDFIEGVI YNASGAVDLK YTYFP* |

A predicted signal peptide is highlighted.

The cp6468 nucleotide sequence <SEQ ID 26> is:

| | |
|---|---|
| 1 | ATGTTTTCAC GATGGATCAC CCTCTTTTTA TTATTCATTA GCCTTACTGG |
| 51 | ATGCTCCTCC TACTCTTCAA AACATAAACA ATCTTTAATT ATTCCCATAC |
| 101 | ATGACGACCC TGTAGCTTTT TCTCCTGAAC AAGCAAAACG GGCCATCGAC |
| 151 | CTTTCTATTG CCCAACTTCT TTTTGATGGT CTGACTAGAG AAACTCATCG |
| 201 | CGAATCCAAT GATTTGGAAT TAGCGATTGC CAGTCGCTAT ACAGTCTCTG |
| 251 | AAGACTTTTG CTCTTATACG TTCTTTATCA AAGACAGCGC TTTATGGAGC |
| 301 | GACGGAACAC CAATCACCTC CGAAGATATC CGTAACGCTT GGGAGTATGC |
| 351 | ACAGGAGAAC TCTCCCCACA TACAGATCTT CCAAGGACTT AACTTCTCAA |
| 401 | CTCCTTCATC AAATGCAATT ACGATTCATC TCGACTCGCC CAACCCCGAT |
| 451 | TTTCCTAAGC TTCTTGCCTT TCCTGCATTT GCTATCTTTA AACCAGAAAA |
| 501 | CCCGAAGCTC TTTAGCGGTC CGTATACTCT TGTAGAGTAT TTCCCAGGGC |
| 551 | ATAACATTCA TTTAAAGAAA AACCCTAACT ATTACGACTA CCACTGCGTC |
| 601 | TCCATCAACT CCATCAAACT GCTCATTATT CCTGATATAT ATACAGCCAT |
| 651 | CCACCTCCTA AACAGAGGCA AGGTGGACTG GGTAGGACAA CCCTGGCATC |
| 701 | AAGGGATTCC TTGGGAGCTC CATAAACAAT CGCAATATCA CTACTACACC |
| 751 | TATCCTGTAG AAGGTGCCTT CTCGCTTTGT CTAAATACAA AATCCCCACA |
| 801 | CTTAAATGAT CTTCAAAACA GACATAGACT CGCTACTTGT ATTGATAAAC |
| 851 | GTTCTATCAT TGAAGAAGCT CTTCAAGGAA CCCAACAACC AGCGGAAACA |
| 901 | CTGTCCCGAG GAGCTCCACA ACCAAATCAA TATAAAAAAC AAAAGCCTCT |
| 951 | AACTCCACAA GAAAAACTCG TGCTTACCTA TCCCTCAGAT ATTCTAAGAT |
| 1001 | GCCAACGCAT AGCAGAAATC TTAAAGGAAC AATGGAAAGC TGCTGGAATA |
| 1051 | GATTTAATCC TTGAAGGACT CGAATACCAT CTGTTTGTTA ACAAACGAAA |
| 1101 | AGTCCAAGAC TACGCCATAG CAACACAGAC TGGAGTTGCT TATTACCCAG |
| 1151 | GAGCAAATCT AATTTCTGAA GAAGACAAGC TCCTGCAAAA CTTTGAGATT |
| 1201 | ATCCCGATCT ACTATCTGAG CTATGACTAT CTCACTCAAG ATTTTATAGA |
| 1251 | GGGAGTAATC TATAATGCTT CTCGAGCTGT AGATCTCAAA TATACCTATT |
| 1301 | TCCCCTAG |

The PSORT algorithm predicts that this protein is an outer membrane lipoprotein (0.790).

The protein was expressed in E. coli and purified as a GST-fusion product, as shown in FIG. 13A. The recombinant protein was used to immunise mice, whose sera were used in a Western blot (FIG. 13B) and for FACS analysis. A his-tagged protein was also expressed.

These experiments show that cp6468 is a useful immunogen. These properties are not evident from the sequence alone.

### Example 14

The following C. pneumoniae protein (PID 4376469) was expressed <SEQ ID 27; cp6469>:

| | |
|---|---|
| 1 | MKMHRLKPTL KSLIPNLLFL LLTLSSCSKQ KQEPLGKRLV IAMSHDLADL |
| 51 | DPRNAYLSRD ASLAKALYEG LTRETDQGIA LALAESYTLS KDRKVYTFKL |
| 101 | RPSVWSDGTP LTAYDFEKSI KQLYFEEFSP SIHTLLGVIK NSSAIHIAQK |
| 151 | SLETLGIQAK DDLTLVITLE QPFPYFLTLI ARPVFSPVHH TLRESYRKGT |
| 201 | PPSTYISNGP FVLKKHEHQN YLILEKNPHY YDHESVKLDR VTLKIIPDAS |
| 251 | TATKLFKSKS IDWIGSPWSA PISNEDQKVL SQEKILTYSV SSTTLLIYNL |
| 301 | QKPLIQNKAL RKAIAHAIDR KSILRLVPSG QEAVTINPPN LSQLNLQKEI |
| 351 | STEERQTKAR AYFQEAKETL SEKELAELSI LYPIDSSNSS IIAQEIQRQL |
| 401 | KDTLGLKIKI QGMEYHCFLK KRRQGDFFIA TGGWIAEYVS PVAPLSILGN |
| 451 | PEDLTQWRNS DYEKTLEKLY LPHAYKENLK KAEMIIEEET PIIPLYHGKY |
| 501 | IYAIHPKIQN TFGSLLGHTD LKNIDILS* |

A predicted signal peptide is highlighted.

The cp6469 nucleotide sequence <SEQ ID 28> is:

| | |
|---|---|
| 1 | ATGAAGATGC ATAQGCTTAA ACCTACCTTA AAAAGTCTGA TCCCTAATCT |
| 51 | TCTTTTCTTA TTGCTCACTC TTTCAAGCTG CTCAAAGCAA AAACAAGAAC |
| 101 | CCTTAGGAAA ACATCTCGTT ATTGCGATGA GCCATGATCT CGCCGACCTA |
| 151 | GATCCTCGCA ATCCCTATTT AAGCACAGAT GCTTCCCTAG CAAAAGCCCT |
| 201 | CTATGAAGCA CTGACAAGAG AAACTGATCA AGAAATCGCA CTGGCTCTTG |
| 251 | CAGAAAGTTA TACCCTGTCA AAAGATCATA AGGTCTATAC CTTTAAACTC |
| 301 | AGACCTTCTG TGTGGAGCGA TGGCACTCCA CTCACTGCTT ATGACTTTGA |
| 351 | AAAATCTATA AAACAACTGT ACTTCGAAGA ATTTTCACCT TCCATACATA |
| 401 | CTTTACTCGG CGTGATTAAA AATTCTTCGG CAATCCACAA TGCTCAAAAA |
| 451 | TCTCTGGAAA CTCTTGGGAT ACAGGCAAAA GATGATCTTA CTTTGGTGAT |
| 501 | TACCCTAGAG CAACCTTTCC CATACTTTCT CACACTTATC GCTCGCCCCG |
| 551 | TATTCTCCCC TGTTCATCAC ACCCTTAGGG AACTCCTTAA GAAAGGAACA |
| 601 | CCCCCATCCA CATACATCTC CAATGGGCCC TTTGTCTTAA AAAAACATGA |
| 651 | ACACCAAAAC TACTTAATTT TAGAAAAAAA TCCTCACTAC TATGATCATG |
| 701 | AATCAGTAAA GTTAGACCGA GTCACCTTAA AAATTATCCC AGACGCCTCC |
| 751 | ACAGCCACGA AACTTTTCAA AAGTAAATCT ATAGATTGGA TTGGCTCACC |
| 801 | TTGGAGCGCT CCGATATCTA ACGAAGACCA AAAAGTTCTC TCCCAAGAAA |
| 851 | AGATTCTTAC CTATTCTGTT TCAAGCACCA CCCTTCTTAT CTATAACCTG |
| 901 | CAAAAACCTC TAATACAAAA TAAAGCCCTC AGGAAAGCCA TTGCTCATGC |
| 951 | TATTGATAGA AAATCTATCT TAAGACTCGT GCCTTCAGGA CAAGAAGCTG |
| 1001 | TAACTCTAGT TCCCCCAAAT CTTTCACAAC TCAATCTTCA AAAAGAGATC |
| 1051 | TCAACAGAAG AACGACAAAC AAAAGCCAGA GCATATTTTC AAGAAGCTAA |
| 1101 | AGAAACACTT TCTGAAAAAG AACTCGCAGA ACTCAGCATC CTCTATCCTA |
| 1151 | TAGATTCCTC GAATTCCTCC ATCATAGCTC AAGAAATCCA AAGACAACTT |
| 1201 | AAAGATACcT TAGGATTGAA AATCAAAATC CAAGGCATGG AGTACCACTG |
| 1251 | CTTTTTAAAG AAACGTCGTC AAGGAGATTT CTTCATAGCG ACAGGAGGAT |
| 1301 | GGATTGCGGA ATACGTAAGC CCCGTAGCCT TCCTATCTAT TCTAGGCAAC |
| 1351 | CCCAGAGACC TCACACAATG GAGAAACAGT GATTACGAAA AGACTTTAGA |
| 1401 | GAAACTCTAT CTCCCTCATG CCTACAAAGA GAATTTAAAA CGCGCAGAAA |
| 1451 | TGATAATAGA AGAAGAAACC CCGATTATCC CCCTGTATCA CGGCAAATAT |
| 1501 | ATTTACGCTA TACATCCTAA AATCCAGAAT ACATTCGGAT CTCTTCTAGG |
| 1551 | CCACACAGAT CTCAAAAATA TCGATATCTT AAGTTAG |

The PSORT algorithm predicts a periplasmic location (0.934).

The protein was expressed in E. coli and purified as a GST-fusion product, as shown in FIG. 14A. The recombinant protein was used to immunise mice, whose sera were used in a Western blot (FIG. 14B) and for FACS analysis. A his-tagged protein was also expressed.

These experiments show that cp6469 is a useful immunogen. These properties are not evident from the sequence alone.

### Example 15

The following C. pneumoniae protein (PID 4376602) was expressed <SEQ ID 29; cp6602>:

| | |
|---|---|
| 1 | MAASGGTGGL GGTQGVNLAA VEAAAAKADA AEVVASQEGS EMNMIQQSQD |
| 51 | LTNPAAATRT KKKEEKFQTL ESRKKGEAGK AEKKSESTEE KPDTDLADKY |
| 101 | ASGNSEISGQ ELRGLRDAIG DDASPEDILA LVQEKIKDPA LQSTALDYLV |
| 151 | QTTPPSQGKL KEALIQARNT HTEQFGRTAI GAKNILFASQ EYADQLNVSP |
| 201 | SGLRSLYLEV TGDTHTCDQL LSMLQDRYTY QDMAIVSSFL MKGMATELKR |
| 251 | QGPYVPSAQL QVLMTETRNL QAVLTSYDYF ESRVPILLDS LKAEGIQTPS |
| 301 | DLNFVKVAES YHKIINDKFP TASKVEREVR NLIGDDVDSV TGVLNLFFSA |
| 351 | LRQTSSRLFS SADKRQQLGA MIANALDAVN INNEDYPKAS DFPKPYPWS* |

The cp6602 nucleotide sequence <SEQ ID 30> is:

| | |
|---|---|
| 1 | ATGGCAGCAT CAGGAGGCAC AGGTGGTTTA GGAGGCACTC AGGGTGTCAA |
| 51 | CCTTGCAGCT GTAGAAGCTG CAGCTGCAAA AGCAGATGCA GCAGAAGTTG |
| 101 | TAGCCAGCCA AGAAGGTTCT GAGATGAACA TGATTCAACA ATCTCAGGAC |
| 151 | CTGACAAATC CCGCAGCAGC AACACGCACG AAAAAAAAGG AAGAGAAGTT |
| 201 | TCAAACTCTA GAATCTCGGA AAAAAGGAGA AGCTGGAAAG GCTGAGAAAA |
| 251 | AATCTGAATC TACAGAAGAG AAGCCTGACA CAGATCTTGC TGATAAGTAT |
| 301 | GCTTCTGGGA ATTCTGAAAT CTCTGGTCAA GAACTTCGCG GCCTGCGTGA |
| 351 | TGCAATAGGA GACGATGCTT CTCCAGAAGA CATTCTTGCT CTTGTACAAG |

| | |
|---|---|
| 401 | AGAAAATTAA AGACCCAGCT CTGCAATCCA CAGCTTTGGA CTACCTGGTT |
| 451 | CAAACGACTC CACCCTCCCA AGGTAAATTA AAAGAAGCGC TTATCCAAGC |
| 501 | AAGGAATACT CATACGGAGC AATTCGGACG AACTGCTATT GGTGCGAAAA |
| 551 | ACATCTTATT TGCCTCTCAA GAATATGCAG ACCAACTGAA TGTTTCTCCT |
| 601 | TCAGGGCTTC GCTCTTTGTA CTTAGAAGTG ACTGGAGACA CACATACCTG |
| 651 | TGATCAGCTA CTTTCTATGC TTCAAGACCG CTATACCTAC CAAGATATGG |
| 701 | CTATTGTCAG CTCCTTTCTA ATGAAAGGAA TGGCAACAGA ATTAAAAAGG |
| 751 | CAGGGTCCCT ACGTACCCAG TGCGCAACTA CAAGTTCTCA TGACAGTAAC |
| 801 | TCGTAACCTG CAAGCAGTTC TTACCTCCTA CGATTACTTT GAAAGTCGCG |
| 851 | TTCCTATTTT ACTCGATAGC TTAAAAGCTG AGGGAATCCA AACTCCTTCT |
| 901 | GATCTAAACT TTGTGAAGGT AGCTGAGTCC TACCATAAAA TCATTAACGA |
| 951 | TAAGTTCCCA ACAGCATCTA AAGTAGAACG AGAAGTCCGC AATCTCATAG |
| 1001 | GAGACGATGT TGATTCTGTG ACCGGTGTCT TGAACTTATT CTTTTCTGCT |
| 1051 | TTACGTCAAA CGTCGTCACG CCTTTTCTCT TCAGGAGACA AACGTCAGCA |
| 1101 | ATTAGGAGCT ATGATTGCTA ATGCTTTAGA TGCTGTAAAT ATAAACAATG |
| 1151 | AAGATTATCC CAAAGCATCA GACTTCCCTA AACCCTATCC TTGGTCATGA |

The PSORT algorithm predicts a cytoplasmic location (0.080).

The protein was expressed in E. coli and purified as both a His-tag and a GST-fusion product, as shown in FIG. 15A. The recombinant proteins were used to immunise mice, whose sera were used in a Western blot (FIG. 15B) and for FACS analysis (FIG. 15C).

The cp6602 protein was also identified in the 2D-PAGE experiment (Cpn0324).

These experiments show that cp6602 is a surface-exposed and immunoaccessible protein, and that it is a useful immunogen. These properties are not evident from the sequence alone.

### Example 16

The following C. pneumoniae protein (PID 4376727) was expressed <SEQ ID 31; cp6727>:

| | |
|---|---|
| 1 | MKYSLPWLLTSSALVFSLHP LMAANTDLSS SDNYENGSSG SAAFTAKETS |
| 51 | DASGTTYTLT SDVSITNVSA ITPADKSCFT NTGGALSFVG ADHSLVLQTI |
| 101 | ALTHDGAAIN NTNTALSFSG FSSLLIDSAP ATGTSGGKGA ICVTNTEGGT |
| 151 | ATFTDNASVT LQKNTSEKDG AAVSAYSIDL AKTTTAALLD QNTSTKNGGA |
| 201 | LCSTANTTVQ GNSGTVTFSS NTATDKGGGI YSKBKDSTLD ANTGVVTFKS |
| 251 | NTAKTGGAWS SDDNLALTGN TQVLFQENKT TGSAAQANNP EGCGGAICCY |
| 301 | LATATDKTGL AISQNQENSF TSNTTTANGG AIYATKCTLD GNTTLTFDQN |
| 351 | TATAGCGGAI YTETEDFSLK GSTGTVTFST NTAKTGGALY SKGNSSLTGN |
| 401 | TNLLFSGWCA TGPSNSSANQ EGCGGAILAF IDSGSVSDKT GLSIANNQEV |
| 451 | SLTSNAATVS GGAIYATKCT LTGNGSLTFD GNTAGTSGGA IYTETEDFTL |
| 501 | TGSTGTVTFS TNTAKTGGAL YSKGNNSLSG NTNLLFSGNK ATGPSNSSAN |
| 551 | QEGCGGAILS FIESASVSTK KGLWIEDNEN VSLSGNTATV SGGAIYATKC |
| 601 | ALHGNTTLTF DGNTAETAGG AIYTETEDFT LTGSTGTVTF STNTAKTAGA |
| 651 | LHTKGNTSFT KNKALVFSGN SATATATTTT DQBGCGGAIL CNISESDIAT |
| 701 | KSLTLTENES LSFINNTAKR SGGGIYAPKC VIBGSESINF DGNTAETSGG |
| 751 | AIYSKNLSIT ANGPVSFTNN SGGKGQAIYI ADSGELSLEA IDGDITFSGN |
| 801 | RATEGTSTPN SIHLGAGAKI TKLAAAPGHT IYFYDPITME APASGGTIEE |
| 851 | LVINPVVKAI VPPPQPKNGP IASVPVVPVA PANPNTGTIV FSSGKLPSQD |
| 901 | ASIPANTTTI LNQKINLAGG NVVLKEGATL QVYSFTQQPD STVFMDAGTT |
| 951 | LETTTTNNTD GSIDLKNLSV NLDALDGKRM ITIAVNSTSG GLKISGDLKF |
| 1001 | HNNEGSFYDN PGLKANLNLP FLDLSSTSGT VNLDDFNPIP SSMAAPDYGY |
| 1051 | QGSWTLVPKV GAGGKVTLVA EWQALGYTPK PELRATLVPN SLWNAYVNIH |
| 1101 | SIQQEIATAM SDAPSHPGIW IGGIGNAPHO DRQKENAGFR LISRGYIVGG |
| 1151 | SMTTPQEYTF AVAFSQLFGK SKDYVVSDIK SQVYAGSLCA QSSYVIPLHS |
| 1201 | SLRRHVLSKV LPELPGETPL VLHGQVSYGR NHHNMTTKLA NNTQGKSDWD |
| 1251 | SHSFAVEVGG SLPVDLNYRY LTSYSPYVKL QVVSVNQKGF QEVAADPRIF |
| 1301 | DASHLVNVSI PMGLTFKHES AKPPSALLLT LGYAVDAYRD HPHCLTSLTN |
| 1351 | GTSWSTFATN LSRQAFFAEA SGHLKLLHGL DCFASGSCEL RSSSRSYNAN |
| 1401 | CGTRYSF* |

A predicted signal peptide is highlighted.

The cp6727 nucleotide sequence <SEQ ID 32> is:

| | |
|---|---|
| 1 | ATGAAATATT CTTTACCTTG GCTACTTACC TCTTCGGCTT TACTTTTCTC |
| 51 | CCTACATCCA CTAATGGCTG CTAACACGGA TCTCTCATCA TCCGATAACT |
| 101 | ATGAAAATGG TAGTAGTGGT AGCGCAGCAT TCACTGCCAA GGAAACTTCG |
| 151 | GATGCTTCAG GAACTACCTA CACTCTCACT AGCGATGTTT CTATTACGAA |
| 201 | TGTATCTGCA ATTACTCCTG CAGATAAAAG CTGTTTTACA AACACAGGAG |
| 251 | GAGCATTGAG TTTTGTTGGA GCTGATCACT CATTGGTTCT GCAAACCATA |
| 301 | GCGCTTACGC ATGATGGTGC TGCAATTAAC AATACCAACA CAGCTCTTTC |
| 351 | TTTCTCAGGA TTCTCGTCAC TCTTAATCGA CTCAGCTCCA GCAACAGGAA |
| 401 | CTTCGGGCGG CAAGGGTGCT ATTTGTGTGA CAAATACAGA GGGAGGTACT |
| 451 | GCGACTTTTA CTGACAATGC CAGTGTCACC CTCCAAAAAA ATACTTCAGA |
| 501 | AAAAGATGGA GCTGCAGTTT CTGCCTACAG CATCGATCTT GCTAAGACTA |
| 551 | CGACAGCAGC TCTCTTAGAT CAAAATACTA GCACAAAAAA TGGCGGGGCC |
| 601 | CTCTGTAGTA CAGCAAACAC TACAGTCCAA GGAAACTCAG GAACGGTGAC |
| 651 | CTTCTCCTCA AATACTGCTA CAGATAAAGG TGGGGGGATC TACTCAAAAG |
| 701 | AAAAGGATAG CACCCTAGAT GCCAATACAG GAGTCGTTAC CTTCAAATCT |
| 751 | AATACTGCAA AGACGGGGGG TGCTTGGAGC TCTGATGACA ATCTTGCTCT |
| 801 | TACCGGCAAC ACTCAAGTAC TTTTTCAGGA AAATAAAACA ACCGGCTCAG |
| 851 | CAGCACAGGC AAATAACCCG GAAGGTTGTG GTGGGGCAAT CTGTTGTTAT |
| 901 | CTTGCTACAG CAACAGACAA AACTGGATTA GCCATTTCTC AGAATCAAGA |
| 951 | AATGAGCTTC ACTAGTAATA CAACAACTGC GAATGGTGGA GCGATCTACG |
| 1001 | CTACTAAATG TACTCTGGAT GGAAACACAA CTCTTACCTT CGATCAGAAT |
| 1051 | ACTGCGACAG CAGGATGTGG CGGAGCTATC TATACAGAAA CTGAACATTT |
| 1101 | TTCTCTTAAG GGAAGTACGG GAACCGTGAC CTTCAGCACA AATACAGCAA |
| 1151 | AGACAGGCGG CGCCTTATAT TCTAAAGGAA ACAGCTCGCT GACTGGAAAT |
| 1201 | ACCAACCTGC TCTTTTCAGG GAACAAAGCT ACGGGCCCGA GTAATTCTTC |
| 1251 | AGCAAATCAA GAGGGTTGCG GTGGGGCAAT CCTAGCCTTT ATTGATTCAG |
| 1301 | GATCCGTAAG CGATAAAACA GGACTATCGA TTGCAAACAA CCAAGATGTC |
| 1351 | AGCCTCACTA GTAATGCTGC AACAGTAAGT GGTGGTGCGA TCTATGCTAG |
| 1401 | CAAATGTACT CTAACTGGAA ACGGCTCCCT GACCTTTGAC GGCAATTCTG |
| 1451 | CTGGAACTTC AGGAGGGGCG ATCTATACAG AAACTGAAGA TTTTACTCTT |
| 1501 | ACAGGAAGTA CAGGAACCGT GACCTTCAGC ACAAATACAG CAAAGACAGG |
| 1551 | CGGCGCCTTA TATTCTAAAG GCAACAACTC TCTGTCTGGT AATACCAACC |
| 1601 | TGCTCTTTTC AGGGAACAAA GCTACGGGCC CGAGTAATTC TTCAGCAAAT |
| 1651 | CAAGAGGGTT GCGGTGGGGC AATCCTATCG TTTCTTGAGT CAGCATCTGT |
| 1701 | AAGTACTAAA AAAGGACTCT GGATTGAAGA TAACGAAAAC GTGAGTCTCT |
| 1751 | CTGGTAATAC TGCAACAGTA AGTGGCGGTG CGATCTATGC GACCAAGTGT |
| 1801 | GCTCTGCATG GAAACACGAC TCTTACCTTT GATGGCAATA CTGCCGAAAC |
| 1851 | TGCAGGAGGA GCGATCTATA CAGAAACCGA AGATTTTACT CTTACGGGAA |
| 1901 | GTACGGGAAC CGTGACCTTC AGCACAAATA CAGCAAAGAC AGCAGGGGCT |
| 1951 | CTACATACTA AAGGAAATAC TTCCTTTACC AAAAATAAGG CTCTTGTATT |
| 2001 | TTCTGGAAAT TCAGCAACAG CAACAGCAAC AACAACTACA GATCAAGAAG |
| 2051 | GTTGTGGTGG AGCGATCCTC TGTAATATCT CAGAGTCTGA CATAGCTACA |
| 2101 | AAAAGCTTAA CTCTTACTGA AAATGAGAGT TTAAGTTTCA TTAACAATAC |
| 2151 | GGCAAAAAGA AGTGGTGGTG GTATTTATGC TCCTAAGTGT GTAATCTCAG |
| 2201 | GCAGTGAATC CATAAACTTT GATGGCAATA CTGCTGAAAC TTCGGGAGGA |
| 2251 | GCGATTTATT CGAAAAACCT TTCGATTACA GCTAACGGTC CTGTCTCCTT |
| 2301 | TACCAATAAT TCTGGAGGCA AGGGAGGCGC CATTTATATA GCCGATAGCG |
| 2351 | GAGAACTTTC CTTAGAGGCT ATTGATGGGG ATATTACTTT CTCAGGGAAC |
| 2401 | CGAGCGACTG AGGGAACTTC AACTCCCAAC TCGATCCATT TAGGAGCAGG |
| 2451 | GGCTAAGATC ACTAAGCTTG CAGCAGCTCC TGGTCATACG ATTTATTTTT |
| 2501 | ATGATCCTAT TACGATGGAA GCTCCTGCAT CTGGAGGAAC AATAGAGGAG |
| 2551 | TTAGTCATCA ATCCTGTTGT CAAAGCTATT GTTCCTCCTC CCCAACCAAA |
| 2601 | AAATGGTCCT ATAGGTTGAG TGCCTGTAGT CCCTGTAGCA CCTGCAAACC |
| 2651 | CAAACACGGG AACTTTAGTA TTTTCTTCTG GAAAACTCCC CAGTCAAGAT |
| 2701 | GCCTCGATTC CTGCAAATAC TACCACCATA CTGAACCAGA AGATCAACTT |
| 2751 | AGCAGGAGGA AATGTCGTTT TAAAAGAAGG AGCCACCCTA CAAGTATATT |
| 2801 | CCTTCACACA GCAGCCTGAT TCTACAGTAT TCATGGATGC AGGAACGACC |
| 2851 | TTAGAGACCA CGACAACTAA CAATACAGAT GGCAGCATCG ATCTAAAGAA |
| 2901 | TCTCTCTGTA AATCTGGATG CTTTAGATGG CAAGCGTATG ATAACGATTG |
| 2951 | CCGTAAACAG CACAAGTGGG GGATTAAAAA TCTCAGGGGA TCTGAAATTC |
| 3001 | CATAACAATG MGGAAGTTTT CTATGACAAT CCTGGGTTGA AAGCAAACTT |
| 3051 | AAATCTTCCT TTCTTAGATC TTTCTTCTAC TTCAGGAACT GTAAATTTAG |
| 3101 | ACGACTTCAA TCCGATTCCT TCTAGCATGC CTGCTCCGGA TTATGGGTAT |
| 3151 | CAAGGGAGTT GGACTCTGGT TCCTAAAGTA GGAGCTGGAG GGAAGGTGAC |
| 3201 | TTTGGTCGCG GAATGGCAAG CGTTAGGATA CACTCCTAAA CCAGAGCTTC |
| 3251 | GTGCGACTTT AGTTCCTAAT ACCCTTTGGA ATGCTTATGT AAACATCCAT |
| 3301 | TCTATACAGC AGGAGATCGC CACTGCGATG TCGGACGCTC CCTCACATCC |
| 3351 | AGGGATTTGG ATTGGAGGTA TTGGCAACGC CTTCCATCAA GACAATCAAA |
| 3401 | AGGAAAATGC AGGATTCCGT TTGATTTCCA GAGGTTATAT TCTTGGTGGC |
| 3451 | AGCATGACCA CCCCTCAAGA ATATACCTTT GCTGTTACAT TCAGCCAACT |
| 3501 | CTTTGGCAAA TCTAAGGATT ACGTAGTCTC GGATATTAAA TCTCAAATCT |
| 3551 | ATGCAGGATC TCTCTATGCT CAGAGCTCTT AAGTCATTCC CCTGCATAGC |
| 3601 | TCATTACGTC GCCACGTCCT CTCTAAGGTC CTTCCAGAGC TCCCAGGAGA |
| 3651 | AACTCCCCTT GTTCTCCATG GTCAAGTTTC CTATGGAAGA AACCACCATA |
| 3701 | ATATGACGAC AAAGCTTGCG AACAACACAC AAGGGAAATC AGACTGGGAC |
| 3751 | AGCCATAGCT TCGCTGTTGA AGTCGGTGGT TCTCTTCCTG TAGATCTAAA |
| 3801 | CTACAGATAC CTTACCAGCT ACTCTCCCTA TGTGAAACTC CAAGTTGTAA |
| 3851 | GTGTAAATCA AAAAGGATTC CAAGACGTTC CTGCTGATCC ACGTATCTTT |
| 3901 | GACGCTAGCC ATCTGGTCAA CGTGTCTATC CCTATGGGAC TCACCTTCAA |
| 3951 | ACACGAATCA GCAAAGCCCC CCAGTGCTTT GCTTCTTACT TTAGGTTACG |
| 4001 | CTGTAGAAGC TTACCGGGAT CACCCTCACT GCCTGACCTC CTTAACAAAT |
| 4051 | GGCACCTCGT GGTCTACGTT TGCTACTAAC TTATCACGAC AAGCTTTCTT |
| 4101 | TGCTGAGGCT TCTGGACATC TGAAGTAACT TCATGGTCTT GACTGCTTCG |
| 4151 | CTTCTGGAAG TTGTGAACTG CGCAGCTCCT CAAGAAGCTA TAATGCAAAC |
| 4201 | TGTGGAACTC GTTATTCTTT CTAA |

The PSORT algorithm predicts an outer membrane location (0.915).

The protein was expressed in E. coli and purified as a his-tag product, as shown in FIG. 16A. The recombinant protein was used to immunise mice, whose sera were used in a Western blot (FIG. 16B) and for FACS analysis (FIG. 16C). A GST-fusion protein was also expressed.

The cp6727 protein was also identified in the 2D-PAGE experiment (Cpn0444).

These experiments show that cp6727 is a surface-exposed and immunoaccessible protein, and that it is a useful immunogen. These properties are not evident from the sequence alone.

### Example 17

The following C. pneumoniae protein (PID 4376731) was expressed <SEQ ID 33; cp6731>:

| | |
|---|---|
| 1 | MKSSLHWFLISSSLALPLSLNFSAFAAVVE INLGPTNSFS GPGTTTPPAG |
| 51 | TTNADGTTYN LTGDVSITNA GSPTALTASC FKETTGNLSF QGIIGYQFLLQ |
| 101 | NIDAGANTCF TNTAANKLLS FSGFSYLSLI QTTNATTGTG AIKSTGACSI |
| 151 | QSNYSCYFGQ NFSNDNGGAL QGSSISLSLN PNLTPTAKNK TQKGGALYST |
| 201 | GGITINNTLN SASFSENTAA NNGGATTTNA SSFISSNKAI SFINNSVWAT |
| 251 | SATGGAIYCS STSAPKPVLT LSDNGELNPI GNTAITSGGA IYTDNLVLSS |
| 301 | GGPTLFIKNS AIPTAAPLGG AIAIADSGSL SLSALGGDIT FEGNTVVKGA |
| 351 | SSSQTTTRNS INIGNTNAKI VQLRASQGNT IYFYDPITTS ITAALSDALN |
| 401 | LNGPDLAGNP AYQGTIVFSG EKLSEAEAAE ADNLKSTIQQ PLTLAGGQLS |
| 451 | LKSGVTLVAK SFSQSPGSTI LMDAGTTLET ADGITITNLV LNVDSLKBTK |
| 501 | KATLKATQAS QTVTLSGSLS LVDPSGNVYI DVSTNNPQVF SCLTLTADDP |
| 551 | ANIHITDLAA DPLEKNPIHW GYQGNWALWT QEDTATKSKA ATLTWTKTGY |
| 601 | NPNPERRGTL VANTLWGSFV DVRSIQQLVA TKVRQSQETR GIWCEGISNF |
| 651 | FHKDSTKINK GFRHISAGYV VGATTTIASD NLITAAFCQL FGKDRDHFIN |
| 701 | KNXASAYAAS LHLQHIATLS SPSLLRYLPG SESEQPVLFD AQISYIYSKN |
| 751 | TMKTYYTQAP KGESSWYNWG CALELASSLP HTALSHEGLF HAYFPFIKVE |
| 801 | ASYIHQDSFK ERNTTLVRSF DSGDLINVSV PIGITFERFS RNERASYEAT |
| 851 | VIYVADVYRK NPDCTTALLI NNTSWKTTGT NLSRQAGIGR AGIFYAFSPN |
| 901 | LEVTSNLSME IRGSSRSYNA DLGGKFQF* |

A predicted signal peptide is highlighted.

The cp6731 nucleotide sequence <SEQ ID 34> is:

| | |
|---|---|
| 1 | ATGAAATCCT CTCTTCATTG GTTTTTAATC TCGTCATCTT TAGCACTTCC |
| 51 | CTTGTCACTA AATTTCTCTG CGTTTCCTGC TGTTGTTGAA ATCAATCTAG |
| 101 | GACCTACCAA TAGCTTCTCT GGACCAGGAA CCTAAACTCC TCCAGCCCAA |
| 151 | ACAACAATTG CAGATGGAAC TATCTATAAT CTAACAGGGG ATGTCTCAAT |
| 201 | CACCAATGCA GCATCTCCGA CAGCTCTAAC CGCTTCCTGC TTTAAAGAAA |
| 251 | CTACTGGGAA TCTTTCTTTC CAAGGCCACG GCTACCAATT TCTCCTACAA |
| 301 | AATATCGATG CGGCAGCGAA CTGTACCTTT ACCAATACAG CTGCAAACAA |
| 351 | GCTTCTCTCC TTTTCAGGAT TCTCCTATTT GTCACTAATA CAAACCACGA |
| 401 | ATGCTACCAC AGGAACAGGA GCCATCAAGT CCACAGGAGC TTGTTCTATT |
| 451 | CACTCGAACT ATAGTTGCTA CTTTGGCCAA AACTTTTCTA ATGACAATGG |
| 501 | AGGCGCCCTC CAAGGCAGCT CTATCAGTCT ATCGCTAAAC CCCAACCTAA |
| 551 | CGTTTGCCAA AAACAAAGCA ACGCAAAAAG GGGGTGCCCT CTATTCCACG |
| 601 | GGAGGGATTA CAATTAACAA TACGTTAAAC TCAGCATCAT TTTCTGAAAA |
| 651 | TACCCCGGCG AACAATGGCG GAGCCATTTA CACGGAAGCT AGCAGTTTTA |
| 701 | TTAGCAGCAA CAAAGCAATT AGCTTTATAA ACAATAGTGT GACCGCAACC |
| 751 | TCAGCTACAG GGGGAGCCAT TTACTGTAGT AGTACATCAG CCCCCAAACC |
| 801 | AGTCTTAACT CTATCAGACA ACGGGGAACT GATCTTTATA GGAAATACAG |
| 851 | CAATTACTAG TGGTGGGGCG ATTTATACTG ACAATCTAGT TCTTTCTTCT |
| 901 | GGAGGACCTA CGCTTTTTAA AAACAACTCT GCTATAGATA CTGCAGCTCC |
| 951 | CTTAGGAGGA GCAATTGCGA TTGCTGACTC TGGATCTTTG AGTCTTTCGG |
| 1001 | CTCTTGGTGG AGACATCACT TTTGAAGGAA ACACATTAGT CAAAGGAGCT |
| 1051 | TCTTCGAGTC AGACCACTAC CAGAAATTCT ATTAACATCG GAAACACCAA |
| 1101 | TGCTAAGATT GTACAGCTGC GAGCCTCTCA AGGCTATACT ATCTACTTCT |
| 1151 | ATGATCCTAT TACAACTAGC ATCACTGCAG CTCTCTCAGA TGCTCTAAAC |
| 1201 | TTAAATGGTC CTGACCTTGC AGGGAATCCT GCATATCAAG GAACCATCGT |
| 1251 | ATTTTCTGGA GAGAAGCTCT CGGAAGCAGA AGCTGCAGAA GCTGATAATC |
| 1301 | TCAAATCTAC AATTCAGCAA CCTCTAACTC TTGCGGGAGG GCAACTCTCT |
| 1351 | CTTATATCAG GACTCACTCT AGTTGCTATG TCCTTTTCGC AATCTCCGGG |
| 1401 | CTCTACCCTC CTCATGGATG CATGGTCCAC ATTAGAAACC GCTGATGGGA |
| 1451 | TCACTATCAA TAATCTTGTT CTCAATGTAG ATTCCTTAAA AGATACCAAG |
| 1501 | AAGGCTACGC TAAAAGCAAC ACAAGCAATT CAGACAGTCA CTTTATCTGG |
| 1551 | ATCGCTCTCT CTTGTAGATC CTTCTGGAAA TGTCTACGAA GATGTCTCTT |
| 1601 | GGAATAACCC TCAAGTCTTT TCTTGTCTCA CTCTTACTGC TGACGTCCCC |
| 1651 | GCGAATATTC ACATCACAGA CTTAGCTGCT GATCCCCTAG AAAAAAATCC |
| 1701 | TATCCATTGG GGATACCAAG GGAATTGGGC ATTATCTTGG CAAGAGGATA |
| 1751 | CTGCGACTAA ATCCAAAGCA GCGACTCTTA CCTGGACAAA AACAGGATAC |
| 1801 | AATCCGAATC CTGAGCGTCG TGGAACCTTA GTTGCTAACA CGCTATGGGG |
| 1851 | ATCCTTTGTT GATGTGCGCT CCATACAACA GCTTGTAGCC ACTAAAGTAC |
| 1901 | GCCAATCTCA AGAAACTCGC GGCATCTGGT GTGAAGGGAT CTCGAACTTC |
| 1951 | TTCCATAAAG ATAGCACGAA GATAAATAAA GGTTTTCGCC ACATAAGTGC |
| 2001 | AGGTTATGTT GTAGGAGCGA CTACAACATT AGCTTCTGAT AATCTTATCA |
| 2051 | CTGCAGCCTT CTGCCAATTA TTCGGGAAAG ATAGAGATCA CTPTATAAAT |
| 2101 | AAAAATAGAG CTTCTGCCTA TGCAGCTTCP CTCCATCTCC AGCATCTAGC |
| 2151 | GACCTTGTCT TCTCCAAGCT TGTTACGCTA CCTTCCTGGA TCTGAAAGTG |
| 2201 | AGCAGCCTGT CCTCTTTGAT GCTCAGATCA GCTATATCTA TAGTAAAAAT |
| 2251 | ACTATGAAAA CCTATTACAC CCAAGCACCA AAGGGAGAGA GCTCGTGGTA |
| 2301 | TAATGACGGT TGCGCTCTGG AACTTGCGAG CTCCCTACCA CACACTGCTT |
| 2351 | TAAGCCATGA GGGTCTCTTC CACGCGTATT TTCCTTTCAT CAAAGTAGAA |
| 2401 | GCTTCGTACA TACACCAAGA TAGCTTCAAA GAACGTAATA CTACCTTGGT |
| 2451 | ACGATCTTTC GATAGCGGTG ATTTAATTAA CGTCTCTGTG CCTATTGGAA |
| 2501 | TTACCTTCGA GAGATTCTCG AGAAACGAGC GTGCGTCTTA CGAAGCTACT |
| 2551 | GTCATCTACG TTGCCGATGT CTATCGTAAG AATCCTGACT GCACGACAGC |
| 2601 | TCTCCTAATC AACAATACCT CGTGGAAAAC TACAGGAACG AATCTCTCAA |
| 2651 | GATAAGCTGG TATCGGAAGA GCAGGGATCT TTTATGCCTT CTCTCCAAAT |
| 2701 | TTTGAGGTCA CAAGTAACCT ATCTATGGAA ATTCGTGGAT CTTCACGCAG |
| 2751 | CTACAATGCA GATCTTGGAG GTAAGTTCCA GTTCTAA |

The PSORT algorithm predicts an outer membrane location (0.926).

The protein was expressed in E. coli and purified as a his-tag product, as shown in FIG. 17A. A GST-fusion protein was also expressed. The recombinant proteins were used to immunise mice, whose sera were used in a Western blot (FIG. 17B; his-tag) and for FACS analysis (FIG. 17C; his-tag and GST-fusion).

The GST-fusion protein also showed good cross-reactivity with human sera, including sera from patients with pneumonitis. Less cross-reactivity was seen with the his-fusion.

| | |
|---|---|
| 1701 | TCTTTATGAT ATGGTGTCAT TACAAACTCC AGTAGCAATT CCTATCGCTG |
| 1751 | TTTTCAAAGG AGCAACCGTT ACTAAGACAG GATTTCCTGA TGGGGAGATT |
| 1801 | GCGACTCCAA GCCACTACGG CTACCAAGGA AAGTGGTCCT ACACATGGTC |
| 1851 | CCGTCCCCTG TTAATTCCAG CTCCTGATGG AGGATTTCCT GGAGGTCCCT |
| 1901 | CTCCTAGCGC AAATACTCTC TATGCTTTAT GGAATTCAGA CACTCTCGTG |
| 1951 | CGTTCTACCT ATATCTTAGA TCCCGAGCGT TACGGAGAAA TTGTCAGCAA |
| 2001 | CAGCTTATGG ATTTCCTTCT TAGGAAATCA GGCATTCTCT GATATTCTCC |
| 2051 | AAGATGTTCT TTTGATAGAT CATCCCGGGT TGTCCATAAC CGCGAAAGCT |
| 2101 | TTAGGAGCCT ATGTCGAACA CACACCAAGA CAAGGACATG AGGGCTTTTC |
| 2151 | AGGTCGCTAT GGAGGCTACC AAGCTGCGCT ATCTATGAAC TACACGGACC |
| 2201 | ACACTACGTT AGGACTTTCT TTCGGGCAGC TTTATGGAAA AACTAACGCC |
| 2251 | AACCCCTACG ATTCACGTTG CTCAGAACAA ATGTATTTAC TCTCGTTCTT |
| 2301 | TGGTCAATTC CCTTTCGTGA CTCAAAAGAG CGAGGCCTTA ATTTCCTGGA |
| 2351 | AAGCAGCTTA TGGTTATPCC AAAAATCACC TAAATACCAC CTACCTCAGA |
| 2401 | CCTGACAAAG CTCCAAAATC TCAAGGGCAA TGGCATAACA ATAGTTACTA |
| 2451 | TGTTCTTATT TCTGCAGAAC ATCCTTTCCT AAACTGGTGT CTTCTTACAA |
| 2501 | GACCTCTGGC TCAAGCTTGG GATCTTTCAT GTTTTATTTC CGCAGAATTC |
| 2551 | CTAGGTGGTT GGCAAAGTAA GTTCACAGAA ACTGGAGATC TGCAACGTAG |
| 2601 | CTTTAGTAGA GGTAAAGGGT ACAATGTTTC CCTACCGATA GGATGTTCTT |
| 2651 | CTCAATGGTT CACACCATTT AAGAAGGCTC CTTCTACACT GACCATCAAA |
| 2701 | CTTGCCTACA AGCCTGATAT CTATCGTGTC AACCCTCACA ATATTGTGAC |
| 2751 | TGTCGTCACA AACCAATATA GCACTTCGAT CTCATGAGCA AATCTACGCC |
| 2801 | GCCACGGTTT GTTTGTACAT ATCCATGATG TATTAGATCT CACCGAGGAC |
| 2851 | ACTCAGGCCT TTCTAAACTA TACCTTTGAC GGGAAAAATG GATTTACAAA |
| 2901 | CCACCGAGTG TCTACAGGAC TAAAATCCAC ATTTTAA |

The PSORT algorithm predicts an outer membrane location (0.940).

The protein was expressed in E. coli and purified as a GST-fusion product, as shown in FIG. 18A. The recombinant protein was used to immunise mice, whose sera were used in an immunoblot analysis blot (FIG. 18B) and for FACS analysis (FIG. 18C). A his-tagged protein was also expressed.

The cp6737 protein was also identified in the 2D-PAGE experiment (Cpn0454) and showed good cross-reactivity with human sera, including sera from patients with pneumonitis.

These experiments show that cp6737 is a surface-exposed and immunoaccessible protein, and that it is a useful immunogen. These properties are not evident from the sequence alone.

### Example 19

The following C. pneumoniae protein (PID 4377090) was expressed <SEQ ID 37; cp7090>:

| | |
|---|---|
| 1 | MNIHSLWKLCTLLALLALPACSLSPNYGWE DSCNTCHHTR RKKPSSFGFV |
| 51 | PLYTEEDFNP NFTFGEYDSK EEKQYKSSQV AAFRNITFAT DSYTIKGEEN |
| 101 | LAILTNLVHY MKKNPKATLY IEGHTDERGA ASYNLALGAR RANAIKEHLR |
| 151 | KQGISADRLS TISYGKERPL NSGHNELAWQ QNFRTEFKIH AR* |

A predicted signal peptide is highlighted.

The cp7090 nucleotide sequence <SEQ ID 38> is:

| | |
|---|---|
| 1 | ATGAATATAC ATTCCCTATG GAAACTTTGT ACTTTATTGG CTTTACTTGC |
| 51 | ATTGCCAGCA TGTAGCCTTT CCCCTAATTA TGGCTGGGAG GATTCCTGTA |
| 101 | ATACATGCCA TCATACATGA CGAAAAAAGC CTTCTTCTTT TGGCTTTGTT |
| 151 | CCTCTCTATA CCGAAGAGGA CTTTTACCCT AATTTTACCT TCGGTGAGTA |
| 201 | TGATTCCAAA GAAGAAAAAC TATACAAGTC AAGCCAAGTT GCAGCATTTC |
| 251 | GTAATATCAC CTTTGCTACA GACAGCTATA CAATTTAAGG TGAAGTGAAC |
| 301 | CTTGCGATTC TCACGAACTT GGTTCACTAC ATGAAGAAAA ACCCGAAAGC |
| 351 | TACACTGTAC ATTGAAGGGC ATACTGACGA GCGTGGAGCA GCATCCTATA |
| 401 | ACCTTGCTTT AGGAGCACGA CGAGCCAATG CGATTAAAGA GCATCTCCGA |
| 451 | AAGCAGGGAA TCTCTGCAGA TCGTCTATCT ACTATTTCCT ACGGAAAAGA |
| 501 | ACATCCTTTA AATTCGGGAC ACAACGAACT AGCATGGCAA CAAAATCGCC |
| 551 | GTACAGAGTT TAAGATTCAT GCACGCTAA |

The PSORT algorithm predicts an outer membrane location (0.790).

The protein was expressed in E. coli and purified as a GST-fusion product, as shown in FIG. 19A. A his-tagged protein was also expressed. The recombinant proteins were used to immunise mice, whose sera were used in a Western blot FIG. 19B) and for FACS analysis.

These experiments show that cp7090 is useful immunogen. These properties are not evident from the sequence alone.

### Example 20

The following C. pneumoniae protein (PID 4377091) was expressed <SEQ ID 39; cp7091>:

| | |
|---|---|
| 1 | MLRQLCFQVF FFCFASLVYA EELEVVVRSE HITLPIEVSC QTDTKDPKIQ |
| 51 | KYLSSLTEIF CKDIALGDCL QPTAASKESS SPLAISLRLH VPQLSVVLLQ |
| 101 | SSKTPQTLCS FTISQNLSVD RQKIHHAADT VHYALTGIPG ISAGKIVFAL |
| 151 | SSLGKDQKLK QGEIMTTDYD GKNLAPLTTE CSLSITPKWV GVGSNPPYLY |
| 201 | VSYKYGVPKI FLGSLENTEG KKVLPLKGNQ LMPTFSPRKK LLAFVADTYG |
| 251 | NPDLFIQPFS LTSGPMGRPR RLLNENPGTQ GNPSFNPEGS QLVFISNKDG |
| 301 | RPRLYIMSLD PEPQAPRLLT KKYRNSSCPA WSPDGKKIAF CSVIKGVRQI |
| 351 | CIYDLSSGED YQLTTSPTNK ESPSWAIDSR HLVFSAGNAB ESELYLISLV |
| 401 | TKKTNKIAIG VGEKRFPSWG AFPQQPXKRT L* |

A predicted signal peptide is highlighted.

The cp7091 nucleotide sequence <SEQ ID 40> is:

| | |
|---|---|
| 1 | ATGTTACGGC AACTATGCTT CCAAGTTTTT TTCTTTTGCT TCGCATCGCT |
| 51 | AGTCTATGCT GAAGAATTAG AAGTTGTTGT CCGTTCCGAA CATATCACGC |
| 101 | TCCCTATTGA GGTCTCTTGC CAGACCGATA CGAAAGATCC AAAAATACAG |
| 151 | AAATACCTCA GCTCGCTAAC GGAGATATTT TGCAAGQACA TTGCCCTAGG |
| 201 | AGATTGTCTA CAACCCACAG CGGCTTCTAA AGAATCGTCA TCTCCTTTAG |
| 251 | CAATATCTTT ACGGTTGCAT GTACCTCAGC TATCTGTAGT GCTTTTACAG |
| 301 | TCTTCAAAAA CTCCTCAAAC CTTATGTTCT TTTACTATTT CTCAAAATCT |
| 351 | TTCTGTAGAT CGTCAAAAAA TCCATCACGC TGCTGATACA GTTCATTACG |
| 401 | CCCTCACAGG GATTCCTGGA ATCAGTGCTG GGAAAATTGT TTTTGCTCTA |
| 451 | AGTTCTTTAG GAAAAGATCA AAAGCTCAAG CAAGGAGAAT TATGGACTAC |
| 501 | AGAATACGAT GGGAAAAACC TCGCCCCTTT AACCACAGAA TGTTCGCTCT |
| 551 | CTATAACTCC AAAATGGGTG GGTGTGGGAT CAAATTTTCC CTATCTCTAT |
| 601 | GTTTCGTATA ACTATGGTGT GCCTAAAATT TTTCTTGGTT CCCTAGAGAA |
| 651 | CACTGAAGGT AAAAAAGTCC TTCCGTTAAA AGGCAACCAA CTCATGCCTA |
| 701 | CGTTTTCTCC AAGAAAAAAG CTTTTAGCTT TCGTTGCTGA TACGTATGGA |
| 751 | AATCCTGATT TATTTATTCA ACCGTTCTCA CTAACTTCAG GACCTATGGG |
| 801 | TCGCCCACGT CGCCTCCTTA ATGAGAATTT CGGGACTCAA GGGAATCCCT |
| 851 | CCTTCAACCc TGAAGGATCC CAGCTTGTCT TTATATCGAA CAAAGACGGC |
| 901 | CGTCCGCGTC TTTATATTAT GTCCCTCGAT CCTGAACCCC AAGCACCTCG |
| 951 | CTTGCTGACA AAAAAATACA GAAATAGCAG TTGCCCTGCA TGGTCTCCAG |
| 1001 | ATGGTAAAAA AATAGCCTTC TGCTCTGTAA TTAAAGGGGT GCGACAAATT |
| 1051 | TGTATTTACG ATCTCTCCTC TGGAGAGGAT TACCAACTCA CTACGTCTCC |
| 1101 | CACAAATAAA GAGAGTCCTT CTTGGGCTAT AGACAGCCGT CATCTTGTCT |
| 1151 | TTAGTGCGGG GAATGCTGAA GAATCAGAGT TATATTTAAT CAGTCTAGTC |
| 1201 | ACCAAAAAAA CTAACAAAAT TGCTATAGGA GTAGGAGAAA AACCGTTCCC |
| 1251 | CTCCTGGGGT GCTTTCCCTC AGCAACCGAT AAAGAGAACA CTATGA |

The PSORT algorithm predicts an inner membrane location (0.109).

The protein was expressed in E. coli and purified as a GST-fusion product, as shown in FIG. 20A. A his-tagged protein was also expressed. The recombinant proteins were used to immunise mice, whose sera were used in a Western blot (FIG. 20B) and for FACS analysis.

These experiments show that cp6731 is a surface-exposed and immunoaccessible protein, and that it is a useful immunogen. These properties are not evident from the sequence alone.

### Example 18

The following C. pneumoniae protein (PID 4376737) was expressed <SEQ ID 35; cp6737>:

| | |
|---|---|
| 1 | MPLSFKSSSF CLLACLCSAS CAFAETRLGG NFVPPITNQG EEILLTSDFV |
| 51 | CSNFLGASFS SSPINSSSNL SLLGKGLSLT FTSCQAPTNS NYALLSAAET |
| 101 | LTFKNFSSIN FTGNQSTGLG GLIYGKDIVF QSIKDLIFTT NRVAYSPASV |
| 151 | TTSATPAITT VTTGASALQP TDSLTVENIS QSIKFFGNLA NFGSAISSSP |
| 201 | TAVVKFINNT ATMSFSHNFT SSGGGVIYGG SSLLFENNSG CIIFTANSCV |
| 251 | NSLKGVTPSS GTYALGSGGA ICIPTGTPEL KNNQGKCTFS YNGTPNDAGA |
| 301 | IYAETCNIVG NQGALLLDSN TAARNGGAIC AKVLNIQGRG PIEFSRNRAE |
| 351 | KGGAIFIGPS VGDPAKQTST LTILASEGDI AFQGNHLNTK PGIRNAITVE |
| 401 | AGGEIVSLSA QGGSRLVFYD PITHSLPTTS PSNKDITINA NGASGSVVFT |
| 451 | SKGLSSTELL LPANTTTILL GTVKIASGEL KITDNAVVNV LGFATQGSGQ |
| 501 | LTLGSGGTLG LATPTGAPAA VDFTIGKLAF DPPSFLKRDF VSASVNAGTK |
| 551 | NVTLTGALVL DEHDVTDLYD MVSLQTPVAI PIAVFKGATV TKTGFPDGEI |
| 601 | ATPSUYGYQG KWSYTWSRPL LIPAPDGGFP GGPSPSAWTL YAVWNSDTLV |
| 651 | RSTYILDPER YGEIVSNSLW ISPLGNQAFS DILQDVLLID HPGLSITAKA |
| 701 | LGAYVEHTPR QGHEGFSGRY GGYQAALSMN YTDHTTLGLS FGQLYGKTNA |
| 751 | NPYDSRCSEQ MYLLSFFGQF PIVTQKSEAL ISWKAAYGYS KNHLNTTYLR |
| 801 | PDKAPKSQGQ WHNNSYYVLI SAEHPFLNWC LLTRPLAQAW DLSGFISAEF |
| 851 | LGGWQSKFTE TGDLQRSFSR GKGYNVSLPI GCSSQWFTPF KNHLNTTYLR |
| 901 | LAYKPDIYRV NPHNIVTVVS NQESTSISGA NLRRHGLFVQ IHDVVDLTED |
| 951 | TQAFLNYTFD GKNGFTNHRV STGLKSTP* |

A predicted signal peptide is highlighted.

The cp6737 nucleotide sequence <SEQ ID 36> is:

| | |
|---|---|
| 1 | ATGCCTCTTT CTTTCAAATC TTCATCTTTT TGTCTACTTG CCTGTTTATG |
| 51 | TAGTGCAAGT TGCGCGTTTG CTGAGACTAG ACTCGGAGGG AACTTTGTTC |
| 101 | CTCCAATTAC GAATCAGGGT GAAGAGATCT TACTCACTTC AGATTTTGTT |
| 151 | TGTTCAAACT TCTTGGGGGC GAGTTTTTCA AGTTCCTTTA TCAATAGTTC |
| 201 | CAGCAATCTC TCCTTATTAG GGAAGGGCCT TTCCTTAACG TTTACCTCTT |
| 251 | GTCAAGCTCC TACAAATAGT AACTATGCGC TACTTTCTGC CGCAGAGACT |
| 301 | CTGACCTTCA AGAATTTTTC TTCTATAAAC TTTACAGGGA ACCAATCGAC |
| 351 | AGGACTTGGC GGCCTCATCT ACGGAAAAGA TATTGTTTTC CAATCTATCA |
| 401 | AAGATTTGAT CTTCACTACG AACCGTGTTG CCThTTCTCC AGCATCTGTA |
| 451 | ACTACGTCGG CAACTCCCGC AATCACTACA GTAACTACAG GTGCCTCTGC |
| 501 | TCTCCAACCT ACAGACTCAC TCACTGTCGA AAACATATCC CAATCGATCA |
| 551 | AGTTTTTTGG GAACCTTGCC AACTTCGGCT CTGCAATTAG CAGTTCTCCC |
| 601 | ACGGCAGTCG TTAAATTCAT CAATTACACC GCTACCATGA GCTTCTCCCA |
| 651 | TAACTTTACT TCGTCAGGAG GCGGCGTGAT TTATGGAGGA AGCTCTCTCC |
| 701 | TTTTTGAAAA CAATTCTGGA TGCATCATCT TCACCGCCAA CTCCTGTGTG |
| 751 | AACAGCTTAA AAGGCGTCAC CCCTTCATCA GGAACCTAGG CTTTAGGAAG |
| 801 | TGGCGGAGCC ATCTGCATCC CTACGGGAAC TTTCGAATTA AAAAACAATC |
| 851 | AGGGGAAGTG CACCTTCTCT TATAATGGTA CACCAAATGA TGCGGGTGCG |
| 901 | ATCTACGCCG AAACCTGCAA CATCGTAGGG AACCAGGGTG CCTTGCTCCT |
| 951 | AGATAGCAAC ACTGCAGCGA GAAATGGCGG AGCCATCTGT GCTAAAGTGC |
| 1001 | TCAATATTCA AGGACGCGGT CCTATTGAAT TCTCTAGAAA CCGCGCGGAG |
| 1051 | AAGGGTGGAG CTATTTTCAT AGGCCCCTCT GTTGGAGACC CTGCGAAGCA |
| 1101 | AACATCGACA CTTACGATTT TGGCTTCCGA AGGTGATATT GCGTTCCAAG |
| 1151 | GAAACATGCT CAATACAAAA CCTGGAATCC GCAATGCCAT CACTGTAGAA |
| 1201 | GCAGGGGGAG AGATTGTGTC TCTATCTGCA CAAGGAGGCT CACGTCTTGT |
| 1251 | ATTTTATGAT CCCATTACAC ATAGCCTCCC AACCACAAGT CCGTCTAATA |
| 1301 | AAGACATTAC AATCAACGCT AATGGCGCTT CAGGATCTGT AGTCTTTACA |
| 1351 | AGTAAGGGAC TCTCCTCTAC AGAACTCCTG TTGCCTGCCA ACACGACAAC |
| 1401 | TATACTTCTA GGAACAGTCA AGATCGCTAG TGGAGAACTG AAGATTACTG |
| 1451 | ACAATGCGGT TGTCAATGTT CTTGGCTTCG CTACTCAGGG CTCAGGTCAG |
| 1501 | CTTACCCTGG GCTCTGGAGG AACCTTAGGG CTGGCAACAC CCACGTGAGC |
| 1551 | ACCTGCCGCT GTAGACTTTA CGATTGGAAA GTTAGCATTC GATCCTTTTT |
| 1601 | CCTTCCTAAA AAGAGATTTT GTTTCAGCAT CAGTAAATGC AGGCACAAAA |
| 1651 | AACGTCACTT TAACAGGAGC TCTGGTTCTT GATGAACATG ACGTTACAGA |

These experiments show that cp7091 is a useful immunogen. These properties are not evident from the sequence alone.

### Example 21

The following C. pneumoniae protein (PID 4376260) was expressed <SEQ ID 41; cp6260>:

| | |
|---|---|
| 1 | MRFSLCGFPL VFSFTLLSVF DTSLSATTIS LTPEDSFHGD SQNAERSYNV |
| 51 | QAGDVYSLTG DVSISNVDNS ALNXACDNVT SGSVTFAGNH HGLYFNNISS |
| 101 | GTTKEGAVLC CQDPQATARF SGFSTLSFIQ SPGDIKEQGC LYSKNALMLL |
| 151 | NNYVVRFEQN QSKTRGGAIS GANVTIVCNY DSVSFYQNAA TPGGAIHSSG |
| 201 | PLQIAVNQAE IRFAQNTAXN GSGGALYSDG DIDIDQNAYV LFRENEALTT |
| 251 | AIGKGGAVCC LPPSGSSTPV PIVTFSDNKQ LVFERNHSIM GGGAIYAREL |
| 301 | SISSGGPTLF INNISYANSQ NLGGAIAIDT GGEISLSAEK GTITFQGNRT |
| 351 | SLPFLNGIHL LQNAXFLKLQ ARNGYSIEFY DPITSEADGS TQLNINGDPK |
| 401 | NKEYTGTILF SGEKSLANDP RDFKSTIPQN VNLSAGYLVI KEGABVTVSK |
| 451 | FTQSPGSXLV LDLGTKLIAS KEDIAITGLA IDIDSLSSSS TAAVIKANTA |
| 501 | NKQISVTDSI ELISPTGNAY EDLRMRNSQT FPLLSLEPGA GGSVTVTAGD |
| 551 | FLPVSPHYGF QGNWKLAWTG TGNKVGEFFW DKINYKPRPE KEGNLVPNIL |
| 601 | WGNAVDVRSL MQVQETHASS LQTDRGLWID GIGNFFHVSA SEDNIRYRHN |
| 651 | SGGYVLSVNN EITPKHYTSH AFSQLFSRDK DYAVSNNEYR NYLGSYLYQY |
| 701 | TTSLGNIFRY ASHRPNVNVG ILSRRFLQNP LMIFHFLCAY GRATNIMKTD |
| 751 | YANFPMVKNS WRNNCWAIEC GGSMPLLVFE NGRLPQGAIP FMRLQLVYAY |
| 801 | QGDEKBTTAD GRRFSNGSLT SISVPLGIRF EKLALSQDVL YDPSFSYIPD |
| 851 | IFRKDPSCEA ALVISGDSWL VPAAHVSPHA FVGSGTGRYH FNDYTSLLCR |
| 901 | GSIECRPRAR NYNINCGSKF RF* |

A predicted signal peptide is highlighted.

The cp6260 nucleotide sequence <SEQ ID 42> is:

| | |
|---|---|
| 1 | ATGCGATTTT CGCTCTGCGG ATTTCCTCTA GTTTTTTCTT TTACATTGCT |
| 51 | CTCAGTCTTC GACACTTCTT TGAGTGCTAC TACGATTTCT TTAACCCCAG |
| 101 | AAGATAGTTT TCATGGAGAT AGTCAGAATG CAGAACGTTC TTATAATGTT |
| 151 | CAAGCTGGGG ATGTCTATAG CCTTACTGGT GATGTCTCAA TATCTAACGT |
| 201 | CGATAACTCT GCATTAAATA AAGCCTGCTT CAATGTGACC TCAGGAAGTG |
| 251 | TGACGTTCGC AGGAAATCAT CATGGGTTAT ATTTTAATAA TATTTCCTCA |
| 301 | GGAACTACAA AGGAAGGGGC TGTACTTTGT TGCCAAGATC CTCAAGCAAC |
| 351 | GGCACGTTTT TCTGGGTTCT CCACGCTCTC TTTTATTCAG ACCCCCGGAG |
| 401 | ATATTAAAGA ACAGGGATGT CTCTATTCAA AAAATGCACT TATGCTCTTA |
| 451 | AACAATTATG TAGTGCGTTT TGAACAAAAC CAAAGTAAGA CTAAAGGCGG |
| 501 | AGCTATTAGT GGGGCGAATG TAACTATAGT AGGCAACTAC GATTCCGTCT |
| 551 | CTTTCTATCA GAATGCAGCC ACTTTTGGAG GTGCTATCCA TTCTTCAGGT |
| 601 | CCCCTACAGA TTGCAGTAAA TCAGGCAGAG ATAAGATTTG CACAAAATAC |
| 651 | TGCCAAGAAT GGTTCTGGAG GGGCTTTGTA CTCCGATGGT GATATTGATA |
| 701 | TTGATCAGAA TGCTTATGTT CTATTTCGAG AAAATGAGGC ATTGACTACT |
| 751 | GCTATAGGTA AGGGAGGGGC TGTCTGTTGT CTTCCCACTT CAGGAAGTAG |
| 801 | TACTCCAGTT CCTATTGTGA CTTTCTCTGA CAATAAACAG TTAGTCTTTG |
| 851 | AAAGAAACCA TTCCATAATG GGTGGCGGAG CCATTTATGC TATCAAACTT |
| 901 | AGCATCTCTT CAGGAGGTCC TACTCTATTT ATCAATAATA TATCATATGC |
| 951 | AAATTCGCAA AATTTAGGTG GAGCTATTGC CATTGATACT GGAGGGGAGA |
| 1001 | TCAGTTTATC AGCAGAGAAA GGAACAATTA CATTCCAAGG AAACCGGACG |
| 1051 | AGCTTACCGT TTTTGAATGG CATCCATCTT TTACAAAATG CTAAATTCCT |
| 1101 | GAAATTACAG GCGAGAAATG GATACTCTAT AGAATTTTAT GATCCTATTA |
| 1151 | CTTCTGAAGC AGATGGGTCT ACCCAATTGA ATATCAACGG AGATCCTAAA |
| 1201 | AATAAAGAGT ACACAGGGAC CATACTCTTT TCTGGAGAAA AGAGTCTAGC |
| 1251 | AAACGATCCT AGGGATTTTA AATCTACAAT CCCTCAGAAC GTCAACCTGT |
| 1301 | CTGCAGGATA CTTAGTTATT AAAGAGGGGG CCGAAGTCAC AGTTTCAAAA |
| 1351 | TTCACGCAGT CTCCAGGATC GCATTTAGTT TTAGATTTAG GAACCAAACT |
| 1401 | GATAGCCTCT AAGGAAGACA TTGCCATCAC AGGCCTCGCG ATAGATATAG |
| 1451 | ATAGCTTAAG CTCATCCTCA ACAGCAGCTG TTATTAAAGC AAACACCGCA |
| 1501 | AATAAACAGA TATCCGTGAC GGACTCTATA GAACTTATCT CGCCTACTGG |
| 1551 | CAATGCCTAT GAAGATCTCA GAATGAGAAA TTCACAGACG TTCCCTCTGC |
| 1601 | TCTCTTTAGA GCCTGGAGCC GGGGGTAGTG TGACTGTAAC TGCTGGAGAT |
| 1651 | TTCCTACCGG TAAGTCCCCA TTATGGTTTT CAAGGCAATT GGAAATTAGC |
| 1701 | TTGGACAGGA ACTGGAAACA AAGTTGGAGA ATTCTTCTGG GATAAAATAA |
| 1751 | ATTATAAGCC TAGACCTGAA AAACAAGGAA ATTTAGTTCC TAATATCTTG |
| 1801 | TGGGGGAATG CTGTAGATGT CAGATCCTTA ATGCAGGTTC AAGAGACCCA |
| 1851 | TGCATCGAGC TTACAGACAG ATCGAGGGCT GTGGATCGAT GGAATTGGGA |
| 1901 | ATTTCTTCCA TGTATCTGCC TCCGAAGACA ATATAGGGTA CCGTCATAAC |
| 1951 | AGCGGTGGAT ATGTTCTATC TGTAAATAAT GAGATCACAC CTAAGCACTA |
| 2001 | TACTTCGATG GCATTTTCCC AACTCTTTAG TAGAGACAAG GACTATGCGG |
| 2051 | TTTCCAACAA CGAATACAGA ATGTATTTAG GATCGTATCT CTATCAATAT |
| 2101 | ACAACCTCCC TAGGGAATAT TTTCCGTTAT GCTTCGCGTA ACCCTAATGT |
| 2151 | AAACGTCGGG ATTCTCTCAA GAAGGTTTCT TCAAAATCCT CTTATGATTT |
| 2201 | TTCATTTTTT GTGTGCTTAT GGTCATGCCA CCAATGATAT GAAAACAGAC |
| 2251 | TACGCAAATT TCCCTATGGT GAAAAACAGC TGGAGAAAcA ATTGTTGGGC |
| 2301 | TATAGAGTGC GGAGGGAGCA TGCCTCTATT GGTATTTGAG AACCGAAGAC |
| 2351 | TTTTCCAAGG TGCCATCCCA TTTATGAAAC TACAATTAGT TTATGCTTAT |
| 2401 | CAGGGAGATT TCAAAGAGAC GACTGCAGAT GGCCGTAGAT TTAGTAATGG |
| 2451 | CAGTTTAACA TCGATTTCTG TACCTCTAGG CATACGCTTT GAGAAGCTGG |
| 2501 | CACTTTCTCA GGATGTACTC TATGACTTTA GTTTCTCCTA TATTCCTGAT |
| 2551 | ATTTTCCGTA AGGATCCCTC ATGTGAAGCT GCTCTGGTGA TTAGCGGAGA |
| 2601 | CTCCTGGCTT GTTCCGGCAG CACACGTATC AAGACATGCT TTTGTAGGGA |
| 2651 | GTGGAACGGG TCGGTATCAC TTTAACGACT ATACTGAGCT CTTATGTCGA |
| 2701 | GGAAGTATAG AATGCCGCCC CCATGCTAGG AATTATAATA TAAACTGTGG |
| 2751 | AAGCAAATTT CGTTTTTAG |

The PSORT algorithm predicts an outer membrane location (0.921).

The protein was expressed in E. coli and purified both as a his-tag and GST-fusion product. The GST-fusion is shown in FIG. 21A. This recombinant protein was used to immunise mice, whose sera were used in a Western blot (FIG. 21B) and for FACS analysis (FIG. 21C).

This protein also showed good cross-reactivity with human sera, including sera from patients with pneumonitis.

These experiments show that cp6260 is a surface-exposed and immunoaccessible protein, and that it is a useful immunogen. These properties are not evident from the sequence alone.

### Example 22

The following C. pneumoniae protein (PID 4376456) was expressed <SEQ ID 43; cp6456>:

| | |
|---|---|
| 1 | MSSPVNNTPS APNIPIPAPT TPGIPTTKPR SSFIEKVIIV AKYILFAIAA |
| 51 | TSGALGTILG LSGALTPGIG IALLVIFFVS MVLLGLILKD SISGGEERRL |
| 101 | REEVSRFTSE NQRLTVITTT LETEVKDLKA AKDQLTLEIE AFRNENGNLK |
| 151 | TTABDLEEQV SKLSEQLEAL ERINQLIQAN AGDAQEISSE LKKLISGWDS |
| 201 | KVVEQINTSI QALKVLLGQE WVQEAQTHVK AMQEQIQALQ ABILGMHNQS |
| 251 | TALQKSVENL LVQDQALTRV VGELLESENK LSQACSALRQ EIEKLAQHRT |
| 301 | SLQQRIDAML AQEQNLABQV TALEKMKQEA QKAESEFIAC VRDRTFGRRE |
| 351 | TPPPTTPVVE GDESQEEDEG GTPPVSQPSS PVDRATGDGQ * |

The cp6456 nucleotide sequence <SEQ ID 44> is:

| | |
|---|---|
| 1 | ATGTCATCTC CTGTAAATAA CACACCCTCA GCACCAAACA TTCCAATACC |
| 51 | AGCGCCCACG ACTCCAGGTA TTCCTACAAC AAAACCTCGT TCTAGTTTCA |
| 101 | TTGAAAAGGT TATCATTGTA GCTAAGTACA TACTATTTGC AATTGCAGCC |
| 151 | ACATCAGGAG CACTCGGAAC AATTCTAGGT CTATCTGGAG CGCTAACCCC |
| 201 | AGGAATAGGT ATTGCCCTTC TTGTTATCTT CTTTGTTTCT ATGGTGCTTT |
| 251 | TAGGTTTAAT CCTTAAAGAT TCTATAAGTG GAGGAGAAGA ACGCAGGCTC |
| 301 | AGAGAAGAGG TCTCTCGATT TACAAGTGAG AATCAACGGT TGACAGTCAT |
| 351 | AACCACAACA CTTGAGACTG AAGTAAAGGA TTTAAAAGCA GCTAAAGATC |
| 401 | AACTTACACT TGAAATCGAA GCATTTAGAA ATGAAAACGG TAATTTAAAA |
| 451 | ACAACTGCTG AGGACTTAGA AGAGCAGGTT TCTAAACTTA GCGAACAATT |
| 501 | AGAAGCACTA GAGCGAATTA ATCAACTTAT CCAAGCAAAC GCTGGAGATG |
| 551 | CTCAAGAAAT TTCGTCTGAA CTAAAGAAAT TAATAAGCGG TTGGGATTCC |
| 601 | AAAGTTGTTG AACAGATAAA TACTTCTATT CAAGCATTGA AAGTGTTATT |
| 651 | GGGTCAAGAG TGGGTGCAAG AGGCTCAAAC ACACGTTAAA GCAATGCAAG |
| 701 | AGCAAATTCA AGCATTGCAA GCTGAAATTC TAGGAATGCA CAATCAATCT |
| 751 | ACAGCATTGC AAAAGTCAGT TGAGAATCTA TTAGTACAAG ATCAATCTCT |
| 801 | AACAAGAGWA GTAGGTGAGT TGTTAGAATC TGAGAACAAG CTAAGCCAAG |
| 851 | CTTGTTCTGC GCTACGTCAA GAAATAGAAA AGTTGGCCCA ACATGAAACA |
| 901 | TCTTTGCAAC AACGTATTGA TGCGATGCTA GCCCAAGAGC AAAATTTGGC |
| 951 | AGAGCAGGTC ACAGCCCTTG AAAAAATGAA ACAAGAAGCT CAGAAGGCTG |
| 1001 | AGTCCGAGTT CATTGCTTGT GTACGTGATC GAACTTTCGG ACGTCGTGAA |
| 1051 | ACACCTCCAC CAACAACACC TGTAGTTGAA GGTGATGAAA GTCAAGAAGA |
| 1101 | AGCAGAAGGA GGTACTCCCC CAGTATCACA ACCATCTTCA CCCGTAGATA |
| 1151 | GAGCAACAGG AGATGGTCAG TAA. |

The PSORT algorithm predicts inner membrane (0.127).

The protein was expressed in E. coli and purified as a GST-fusion product, as shown in FIG. 22A. The recombinant protein was used to immunise mice, whose sera were used in a Western blot (FIG. 22B) and for FACS analysis (FIG. 22C). A his-tag protein was also expressed.

These experiments show that cp6456 is a surface-exposed and immunoaccessible protein, and that it is a useful immunogen. These properties are not evident from the sequence alone.

### Example 23

The following C. pneumoniae protein (PID 4376729) was expressed <SEQ ID 45; cp6729>:

| | |
|---|---|
| 1 | MKIPLHKLLI SSTLVTPILL SIATYGADAS LSPTDSFDGA GGSTFTPEST |
| 51 | ADANGTNYVL SGNVYINDAG KGTALTGCCF TETTGDLTFT GKGYSPSFNT |
| 101 | VDAGSNAGAA ASTTADKALT FTGFSNISFI AAPGTTVASG KSTLSSAGAL |
| 151 | NLTDNGTILF SQNVSNEANN NGGAITTKTL SISGNTSSIT FTSNSAKKLG |
| 201 | GAIYSSAAAS ISGNTGQLVF MNNKGETGGG ALGFEASSSI TQNSSLFFSG |
| 251 | NTATDAAGKG GAIYCEKTGE TPTLTISGNK SLTFAENSSV TQGGAICAHG |
| 301 | LDLSAAGPTL FSNNRCGNTA AGKGGAIATA DSGSLSLSAN QGDITFLGNT |
| 351 | LTSPSAPTST RNAIYLGSSA KITNLRAAQG QSIYFYDPIA SNTTGASDVL |
| 401 | TINQPDSNSP LDYSGTIVFS GEKLSADEAK AADNFTSILK QPLALASGTL |
| 451 | ALKGNVELDV NGFTQTEGST LLMQPGTKLK ADTEAXSLTK LVVDLSALEG |
| 501 | NESVSIETAG ANKTITLTSP LVFQDSSGNF YESHTIWQAF TQPLVVFTAA |
| 551 | TAASDIYIDA LLTSPVQTPE PHYGYQGHWE ATWADTSTAK SGTMTWVTTG |
| 601 | YNPNPERRAS VVPDSIMASP TDIRTLQQIM TSQANSIYQQ RGLWASGTAN |
| 651 | FFHKDKSGTN QAFRHKSYGY IVGGSAEDFS ENIFSVAFCQ LFGKDKDLFI |
| 701 | VENTSHNTAA SLYLQHRAFL GGLPMPSFGS ITDMLRDIPL ILNAQLSYSY |
| 751 | TKNDMDTRYT SYPEAQGSWT NNSGALELGG SLALYLPKEA PFFQGYFPFL |
| 801 | EPQAVYSRQQ NFKESGAEAR AFDDGDLVNC SIPVGIRLEK ISEDERNNFE |
| 851 | ISLAYXGDVY RKNPRSRTSL MVSGASWTSL CKNLARQAPL ASAGSHLTAS |
| 901 | PHVSLSGEAA YELRGSAHIY NVDCGLRYSF * |

A predicted signal peptide is highlighted.

The cp6729 nucleotide sequence <SEQ ID 46> is:

| | |
|---|---|
| 1 | ATGAAAATAC CCTTGCACAA ACTCCTGATC TCTTCGACTC TTGTCACTCC |
| 51 | CATTCTATTG AGCATTGCAA CTTACGGAGC AGATGCTTCT TTATCCCCTA |
| 101 | CAGATAGCTT TGATGGAGCG GGCGGCTCTA CATTTACTCC AAAATCTACA |
| 151 | GCAGATGCCA ATGGAACGAA CTATGTCTTA TCAGGAAATG TCTATATAAA |
| 201 | CGATGCTGGG AAAGGCACAG CATTAACAGG CTGCTGCTTT ACAGAAACTA |
| 251 | CGGGTGATCT GACATTTACT GGAAAGGGAT ACTCATTTTC ATTCAACACG |
| 301 | GTAGATGCGG GTTCGAATGC AGGAGCTGCG GCAAGCACAA CTGCTGATAA |
| 351 | AGCCCTAACA TTCACAGGAT TTTCTAACCT TTCCTTCATT GCAGCTCCTG |
| 401 | GAACTACAGT TGCTTCAGGA AAAAGTACTT TAAGTTCTGC AGGAGCCTTA |
| 451 | AATCTTACCG ATAATGGAAC GATTCTCTTT AGCCAAAACG TCTCCAATGA |
| 501 | AGCTAATAAC TATGGCGGAG CGATCACCAC AAAAACTCTT TCTATTTCTG |
| 551 | GGAATACCTC TTCTATAACC TTCACTAGTA ATAGCGCAAA AAAATTAGGT |
| 601 | GGAGCGATCT ATAGCTCTGC GGCTGCAAGT ATTTCAGGAA ACACCGGCCA |
| 651 | GTTAGTCTTT ATGAATAATA AAGGAGAAAC TGGGGGTGGG GCTCTGGGCT |
| 701 | TTGAAGCCAG CTCCTAGATT ACTCAAAATA GCTCCCTTTT CTTCTCTGGA |
| 751 | AACACTGCAA CAGATGCTGC AGGCAAGGGC GGGGCCATTT ATTGTGAAAA |
| 801 | AACAGGAGAG ACTCCTACTC TTACTATCTC TGGAAATAAA AGTCTGACCT |
| 851 | TCGCCGAGAA CTCTTCAGTA ACTCAAGGCG GAGCAATCTG TGCCCATGGT |
| 901 | CTAGATCTTT CCGCTGCTGG CCCTACCCTA TTTTCAAATA ATAGATGCGG |
| 951 | GAACACAGCT GCAGGCAAGQ GCGGCGCTAT TGCAATTGCC GACTCTGGAT |
| 1001 | CTTTAAGTCT CTCTGCAAAT CAAGGAGACA TCACGTTCCT TGGCAACACT |
| 1051 | CTAACCPCAA CCTCCGCCCC AACATCGACA CGGAATGCTA TCTACCTGGG |
| 1101 | ATCGTCAGCA AAAATTACGA ACTTAAGGGC AGCCCAAGGC CAATCTATCT |
| 1151 | ATTTCTATGA TCCGATTGCA TCTAACACCA CAGGAGCTTC AGACGTPCTG |
| 1201 | ACCATCAACC AACCGGATAG CAACTCGCCT TTAGATTATT CAGGAACGAT |
| 1251 | TGTATTTTCT GGGGAAAAGC TCTCTGCAGA TGAAGCGAAA GCTGCTGATA |
| 1301 | ACTTCACATC TATATTAAAG CAACCATTGG CTCTAGCCTC TGGAACCTTA |
| 1351 | GCACTCAAAG GAAATGTCGA GTTAGATGTC AATGGTTTCA CACAGACTGA |
| 1401 | AGGCTCTACA CTCCTCATGC AACCAGGAAC AAAGCTCAAA GCAGATACTG |
| 1451 | AAGCTATCAG TCTTACCAAA CTTGTCGTTG ATCTTTCTGC CTTACAGGGA |
| 1501 | AATAAGAGTG TGTCCATTGA AACAGCAGGA GCCAACAAAA CTATAACTCT |
| 1551 | AACCTCTCCT CTTGTTTTCC AAGATAGTAG CGGCAATTTT TATGAATGCC |
| 1601 | ATACGATAAA CCAAGCCTTC ACGCAGCCTT TGGTGGTATT CACTGCTGCT |
| 1651 | ACTGCTGCTA GCGATATTTA TATCGATGCG CTTCTCACTT CTCCAGTACA |
| 1701 | AACTCCAGAA CCTCATTACG GGTATCAGGG ACATTGGGAA GCCACTTGGG |
| 1751 | CAGACACATC AACTGCAAAA TCAGGAACTA TGACTTGGGT AACTACGGGC |
| 1801 | TACAACCCTA ATCCTGAGCG TAGAGCTTCC GTAGTTCCCG ATTCATTATG |
| 1851 | GGCATCCTTT ACTGACATTC GCACTCTACA GCAGATCATG ACATCTCAAG |
| 1901 | CGAATAGTAT CTATCAGCAA CGAGGACTCT GGGCATCAGG AACTGCGAAT |
| 1951 | TTCTTCCATA AGGATAAATC AGGAACTAAC CAAGCATTCC GACATAAAAG |
| 2001 | CTACGGCTAT ATTGTTGGAG GAAGTGCTGA AGATTTTTCT GAAAATATCT |
| 2051 | TCAGTGTAGC TTTCTGCCAG CTCTTCGGTA AAGATAAAGA CCTGTTTATA |
| 2101 | GTTGAAAATA CCTCTCATAT CTATTTAGCG TCGCTATACC TGCAACATCG |
| 2151 | AGCATTCCTA GGAGGACTTC CCATGCCCTC ATTTGGAAGT ATCACCGACA |
| 2201 | TGCTGAAAGA TATTCCTCTC ATTTTGAATG CCCAGCTAAG CTACAGCTAC |
| 2251 | ACTAAAAATG ATATGGATAC TCGCTATACT TCCTATCCTG AAGCTCAAGG |
| 2301 | CTCTTGGACC AATAACTCTG GGGCTCTAGA GCTCGGAGGA TCTCTGGCTC |
| 2351 | TATATCTCCC TAAAGAAGCA CCGTTCTTCC AGGGATATTT CCCCTTCTTA |
| 2401 | AAGTTCCAGG CAGTCTACAG CCGCCAACAA AACTTTAAAG AGAGTGGCGC |
| 2451 | TGAAGCCCGT GCTTTTGATG ATGGAGACCT AGTGAACTGC TCTATCCCTG |
| 2501 | TCGGCATTCG GTTAGATAAA ATCTCCGAAG ATGAAAAAAA TAATTTCGAG |
| 2551 | ATTTCTCTAG CCTACATPGG TGATGTGTAT CGTAAAAATC CCCGTTCGCG |
| 2601 | TACTTCTCTA ATGGTCAGTG GAGCCTCTTG GACTTCGCTA TGTAAAAACC |
| 2651 | TCGCACGACA AGCCTTCTTA GCAAGTGCTG GAAGCCATCT GACTCTCTCC |
| 2701 | CCTCATGTAG AACTCTCTGG GGAAGCTGCT TATGAGCTTC GTGGCTCAGC |
| 2751 | ACACATCTAC AATGTAGATT GTGGGCTAAG ATACTCATTC TAG |

The PSORT algorithm predicts outer membrane (0.927).

The protein was expressed in E. coli and purified as a GST-fusion product, as shown in FIG. 23A. The recombinant protein was used to immunise mice, whose sera were used in a Western blot (FIG. 23B) and for FACS analysis (FIG. 23C). A his-tag protein was also expressed.

The cp6729 protein was also identified in the 2D-PAGE experiment (Cpn0446) and showed good cross-reactivity with human sera, including sera from patients with pneumonitis.

These experiments show that cp6729 is a surface-exposed and immunoaccessible protein, and that it is a useful immunogen. These properties are not evident from the sequence alone.

### Example 24

The following C. pneumoniae protein (PID 4376849) was expressed <SEQ ID 47; cp6849>:

| | |
|---|---|
| 1 | MSKLIRRVVT VLALTSMASC FASGGIEAAV AESLITKIVA SAETKPAPVP |
| 51 | HTAKKVPLVR RNKQPVEQKS RGAPCDKEFY PCEEGRCQPV EAQQESCYGR |
| 101 | LYSVIWNDDC NVEICQSVPE YATVGSPYPI EILAIGKKDC VDVVITQQLP |
| 151 | CEAEFVSSDP ETTPTSDGKL VWKIDRLGAG DKCKITVWVK PLKEGCCFTA |
| 201 | ATVCACPELR SYTKCGQPAI CIKQEGPDQA CLRCPVCYKI EVVNTGSAIA |
| 251 | RNVTVDNPVP DGYSHASGQR VLSFNLGDMR PGVKKVFTVE FCPQRRGQIT |
| 301 | NVATVTYCGG HXCSAIWTTV VNBPCVQVNI SGADWSYVCK PVEYSISVSN |
| 351 | PGDLVLHDVV IQDTLPSGVT VLEAPGGEIC CNKVVWRIKE MCPGETLQFK |
| 401 | LVVKAQVPGR FTNQVAVTSE SNCGTCTSCA ETTTHWKGLA ATHNCVLDTN |
| 451 | DPICVGENTV YRICVTNRGS AEDTNVSLIL KFSKELQPIA SSGPTKGTIS |
| 501 | GNTVVFDALP KLGSKESVEF SVTLKGIAPG DARGEAILSS DTLTSPVSDT |
| 551 | ENTHVY* |

A predicted signal peptide is highlighted.

The cp6849 nucleotide sequence <SEQ ID 48> is:

| | |
|---|---|
| 1 | ATGTCCAAAC TCATCAGACG AGTAGTTACG GTCCTTGCGC TAACGAGTAT |
| 51 | GGCGAGTTGC TTTGCCAGCG GGGGTATAGA GGCCGCTGTA GCAGAGTCTC |
| 101 | TGATTACTAA GATCGTCGCT AGTGCGGAAA CAAAGCCAGC ACCTGTTCCT |
| 151 | ATGACAGCGA AGAAGGTTAG ACTTGTCCGT AGAAATAAAC AACCAGTTGA |
| 201 | ACAAAAAAGC CGTGGTGCTT TTTGTGATAA AGAATTTTAT CCCTGTGAAG |
| 251 | AGGGACGATG TCAACCTGTA GAGGCTCAGC AAGAGTCTTG CTACGGAAGA |
| 301 | TTGTATTCTG TAAAAGTAAA CGATGATTGC AACGTAGAAA TTTGCCAGTC |
| 351 | CGTTCCAGAA TACGCTACTG TAGGATCTCC TTACCCTATT GAAATCCTTG |
| 401 | CTATAGGCAA AAAAGATTGT GTTGATGTTG TGATTACACA ACAGCTACCT |
| 451 | TGCGAAGCTG AATTCGTAAG CAGTGATCCA GAAACAACTC CTACAAGTGA |
| 501 | TGGGAAATTA GTCTGGAAAA TCGATCGCCT GGGWGCAGGA GATAAATGCA |
| 551 | AAATTACTGT ATGGGTAATA CCTCTTAAAG AAGGTTGCTG CTTCACAGCT |
| 601 | GCTACTGTAT GTGCTTGCCC AGAGCTCCGT TCTTATACTA AAPGCGGTCA |
| 651 | ACCACCATTT TGTATTAAGC AAQAAGGACC TGACTGTGCT TGCCTAAGAT |
| 701 | GCCCTGTATG CTACAAAATC GAAGTAGTGA ACACAGGATC TGCTATTGCC |
| 751 | CGTAACGTAA CTGTAGATAA TCCTGTTCCC GATGGCTATT CTCATGCATC |
| 801 | TGGTCAAAGA GTTCTCTCTT TTAAGTTAGG AGACATGAGA CCTGGCGATA |
| 851 | AAAAGGTATT TACAGTTGAG TTCTGCCCTC AAAGAAGAGG TCAAATCACT |
| 901 | AACGTTGCTA CTGTAACTTA CTGCGGTGGA CACAAATGTT CTGCAAATGT |
| 951 | AACTACAGTT GTTAATGAGC CTTGTGTACA AGTAAATATC TCTGGTGCTG |
| 1001 | ATTGGTCTTA CGTATGTAAA CCTGTGGAGT ACTCTATCTC AGTATCGAAT |
| 1051 | CCTGGAGACT TGGTTCTTCA TGATGTCGTG ATCCAAGATA CACTCCCTTC |
| 1101 | TGGTGTTACA GTACTCGAAG CTCCTGGTGG AGAGATCTGC TGTAATAAAG |
| 1151 | TTGTTTGGCG TATTAAAGAA ATGTGCCCAG GAGAAACCCT CCAGTTTAAA |
| 1201 | CTTGTAGTGA AAGCTCAAGT TCCTGGAAGA TTCACAAATC AAGTTGCAGT |
| 1251 | AACTAGTGAG TCTAACTGCG GAACATGTAC ATCTTGCGCA GAAACAACAA |
| 1301 | CACATTGGAA AGGTCTTGCA GCTACCCATA TGTGCGTMTT AGACACAAAT |
| 1351 | GATCCTATCT GTGTAGGAGA AAATACTGTC TATCGTATCT GTGTAACTAA |
| 1401 | CCGTGGTTCT GCTGAAAATA CTAACGTATC TTTAATCTTG AAGTTCTCAA |
| 1451 | AAGAACTTCA GCCAATAGCT TCTTCAGGTC CAACTAAAGG AACGATTTCA |
| 1501 | GGTAATACCG TTGTTTTCGA CGCTTTACCT AAACTCGGTT CTAAGGAATC |
| 1551 | TGTAGAGTTT TCTGTTACCT TGAAAGGTAT TGCTCCCGGA GATGCTCGCG |
| 1601 | GCGAAGCTAT TCTTTCTTCT GATACACTGA CTTCACCAGT ATCAGACACA |
| 1651 | GAAAATACCC ACGTGTATTA A |

The PSORT algorithm predicts periplasmic space (0.93),

The protein was expressed in E. coli and purified as a GST-fusion product, as shown in FIG. 24A, and also as a his-tag protein. The recombinant proteins were used to immunise mice, whose sera were used in a Western blot (FIG. 24B) and for FACS analysis FIG. 24C).

The cp6849 protein was also identified in the 2D-PAGE experiment (Cpn0557).

These experiments show that cp6849 is a surface-exposed and immunoaccessible protein, and that it is a useful immunogen. These properties are not evident from the sequence alone.

### Example 25

The following C. pneumoniae protein (PID 4376273) was expressed <SEQ ID 49; cp6273>:

| | |
|---|---|
| 1 | MGLFHLTFG LLLCSLPISL VAKFPESVGH KILYISTQST |
| | QQALATYLEA |
| 51 | LDAYGDHDPP VLRKIGEDYL KQSIHSSDPQ TRXSTIIGAG |
| | LAGSSEAKDV |
| 101 | LSQAMETADP LQQLLVLSAV SGHLGKTSDD LLFKALASPY |
| | PVIRLEAAYR |
| 151 | LANLKNTKVI DHLHSFIHKL PEEIQCLSAA IFLRLETEES |
| | DAYIRDLLAA |
| 201 | KKSAIRSATA LQIGEYQQKR FLPTLRNLLT SASPQDQEAI |
| | LYALGKLKDG |
| 251 | QSYYNIKKQL QKPDVDVTLA AAQALIALGK EEDALPVIKX |
| | QALEERPRAL |
| 301 | YALRHLPSEI GIPIALPIFL KTKSNEAKLN VALALLELGC |
| | DTPKLLEYIT |
| 351 | ERLVQPHYNE TLALSFSKGR TLQNWKRVNI IVPQDPQERE |
| | RLLSTTRGLE |
| 401 | EQILTFLFRL PKEAYLPCIY KLLASQKTQL ATTAISFLSH |
| | TSHQEALDLL |
| 451 | FQAAKLPGEP IIBAYADLAI YNLTKDPEKK RSLHDYAKKL |
| | IQETLLFVDT |
| 501 | ENQRPHPSMP YLRYQVTPES RTKLMLDILE TLATSKSSED |
| | IRLLIQLMTE |
| 551 | GDAKNFPVLA GLLIKIVE* |

A predicted signal peptide is highlighted.

The cp6273 nucleotide sequence <SEQ ID 50> is:

| | |
|---|---|
| 1 | ATGGGACTAT TCCATCTAAC TCTCTTTGGA CTTTTATTGT |
| | GTAGTCTTCC |
| 51 | CATTTCTCTT GTTGCTAAAT TCCCTGAGTC TGTAGGTCAT |
| | AAGATCCTTT |
| 101 | ATATAAGTAC GCAATCTACA CAGCAGGCCT TAGCAACATA |
| | TCTGGAAGCT |
| 151 | CTAGATGCCT ACGGTGATCA TGACTTCTTC GTTTTAAGAA |
| | AAATCGGAGA |
| 201 | AGACTATCTC AAGCAAAGCA TCCACTCCTC AGATCCGCAA |
| | ACTAGAAAAA |
| 251 | GCACCATCAT TGGAGCAGGC CTGGCGGGAT CTTCAGAAGC |
| | CTTGGACGTG |
| 301 | CTCTCCCAAG CTATGGAAAC TGCAGACCCC CTGCAGCAGC |
| | TACTGGTTTT |
| 351 | ATCGGCAGTC TCAGGACATC TTGGGAAAAC TTCTGACGAC |
| | TTACTGTTTA |
| 401 | AAGCTTTAGC ATCTCCCTAT CCTGTCATCC GCTTAGAAGC |
| | CGCCTATAGA |
| 451 | CTTGCTAATT TGAAGAACAC TAAAGTCATT GATCATCTAC |
| | ATTCTTTCAT |
| 501 | TCATAAGCTT CCCGAAGAAA TCCAATGCCT ATCTGCGGCA |
| | ATATTCCTAC |
| 551 | GCTTGGAGAC TGAAGAATCT GATGCTTATA TTCGGGATCT |
| | CTTAGCTGCC |
| 601 | AAGAAAAGCG CGATTCGGAG TGCCACAGCT TTGCAGATCG |
| | GAGAATACCA |
| 651 | ACAAAAACGC TTTCTTCCGA CACTTAGGAA TTTGCTAACG |
| | AGTGCGTCTC |
| 701 | CTCAAGATCA AGAAGCTATT CTTTATGCTT TAGGGAAGCT |
| | TAAGGATGGT |
| 751 | CAGAGCTACT ACAATATAAA AAAGCAATTG CAGAAGCCTG |
| | ATGTGGATGT |
| 801 | CACTTTAGCA GCAGCTCAAG CTTTAATTGC TTTGGGGAAA |
| | GAAGAGGACG |
| 851 | CTCTTCCCGT GATAAAAAAG CAAGCACTTG AGGAGCGGCC |
| | TCGAGCCCTG |
| 901 | TATGCCTTAC GGCATCTACC CTCTGAGATA GGGATTCCGA |
| | TTGCCCTGCC |
| 951 | GATATTCCTA AAAACTAAGA ACAGCGAAGC CAAGTTGAAT |
| | GTAGCTTTAG |
| 1001 | CTCTCTTAGA GTTAGGGTGT GACACCCCTA AACTACTGGA |
| | ATACATTACC |
| 1051 | GAAAGGCTTG TCCAACCACA TTATAATGAG ACTCTAGCCT |
| | TGAGTTTCTC |
| 1101 | TAAGGGGCGT ACTTTACAAA ATTGGAAGCG GGTTAACATC |
| | ATAGTCCCTC |
| 1151 | AAGATCCCCA GGAGAGGGAA AGGTTGCTCT CCACAACCCG |
| | AGGTCTTGAA |
| 1201 | GAGCAGATCC TTACGTTTCT CTTCCGCCTA CCTAAAGAAG |
| | CTTACCTCCC |
| 1251 | CTGTATTTAT AAGCTTTTGG CGAGTCAGAA AACTCAGCTT |
| | GCCACTACTG |
| 1301 | CGATTTCTTT TTTAAGTCAC ACCTCACATC AGGAAGCCTT |
| | AGATCTACTT |
| 1351 | TTCCAAGCTG CGAAGCTTCC TGGAGAACCT ATCATCCGCG |
| | CCTATGCAGA |
| 1401 | TCTTGCTATT TATAATCTCA CCAAAGATCC TGAAAAAAAA |
| | CGTTCTCTCC |
| 1451 | ATGATTATGC AAAAAAGCTA ATTCAGGAAA CCTTGTTATT |
| | TGTGGACACG |
| 1501 | GAAAACCAAA GACCCCATCC CAGCATGCCC TATCTACGTT |
| | ATCAGGTCAC |
| 1551 | CCCAGAAAGC CGTACGAAGC TCATGTTGGA TATTCTAGAG |
| | ACACTAGCCA |
| 1601 | CCTCGAAGTC TTCCGAAGAT ATCCGTTTAT TGATACAACT |
| | GATGACGGAA |
| 1651 | GGAGATGCAA AAAATTTCCC AGTCCTTGCA GGCTTACTCA |
| | TAAAAATTGT |
| 1701 | GGAGTAA |

The PSORT algorithm predicts a periplasmic location (0.922).

The protein was expressed in E. coli and purified as a his-tag product and as a GST-fusion product, as shown in FIG. 25A. The recombinant GST-fusion was used to immunise mice, whose sera were used in a Western blot (FIG. 25B) and for FACS analysis (FIG. 25C).

This protein also showed good cross-reactivity with human sera, including sera from patients with pneumonitis.

These experiments show that cp6273 is a surface-exposed and immunoaccessible protein, and that it is a useful immunogen. These properties are not evident from the sequence alone.

### Example 26

The following C. pneumoniae protein (PID 4376735) was expressed <SEQ ID 51; cp6735>:

| | |
|---|---|
| 1 | MTILRNFLTC SALFLALPAA AQVVYLHESD GYNGAINNKS |
| | LEPKITCYPE |
| 51 | GTSYIFLDDV RISNVKHDQE DAGVFINRSG NLFFHGNRON |
| | FTFHNLMTEG |
| 101 | FGAAISNRVG DTTLTLSNFS YLAFTSAPLL PQGQGAIYSL |
| | GSVNIENSEE |
| 151 | VTFCGNYSSW SGAAIYTPYL LGSKASRPSV NLSGNRYLVF |
| | RDNVSQGYGG |
| 201 | AISTHNLTLT TRGPSCFENN HAYHDVNSNG GAIAIAPGGS |
| | ISISVKSGDL |
| 251 | IFKGNTASQD GNTIHNSIHL QSGAQFRNLR AVSESGVYFY |
| | DPISRSESHK |
| 301 | ITDLVINAPE GKETYEGTIS FSGLCLDDHE VCAENLTSTI |
| | LQDVTLAGGT |
| 351 | LSLSDGVTLQ LUSFKQEASS TLTMSPGTTL LCSGDARVQN |
| | LHILIEDTDN |
| 401 | FVPVRIRAED KDALVSLEKL KVAFEAYWSV YDFPQFKEAF |
| | TIPLLELLGP |
| 451 | SFDSLLLGET TLERTQVITE NDAVRGPWSL SWEEYPPSLD |
| | KDRRITPTKK |
| 501 | TVFLTWNPEI TSTP* |

A predicted signal peptide is highlighted.

The cp6735 nucleotide sequence <SEQ ID 52> is:

| | |
|---|---|
| 1 | ATGACCATAC TTCGAAATTT TCTTACCTGC TCGGCTTTAT |
| | TCCTCGCTCT |
| 51 | CCCTGCAGCA GCACAAGTTG TATATCTTCA TGAAAGTGAT |
| | GGTTATAACG |
| 101 | GTGCTATCAA TAATAAAAGC TTAGAACCTA AAATTACCTG |
| | TTATCCAGAA |
| 151 | GGAACTTCTT ACATCTTTCT AGATGACGTG AGGATTTCCA |
| | ACGTTAAGCA |
| 201 | TGATCAAGAA GATGCTGGGG TTTTTATAAA TCGATCTGGG |
| | AATCTTTTTT |
| 251 | TCATGGGCAA CCGTTGCAAC TTCACTTTTC ACAACCTTAT |
| | GACCGAGGGT |
| 301 | TTTGGCGCTG CCATTTCGAA CCGCGTTGGA GACACCACTC |
| | CCACTCTCTC |
| 351 | TAATTTTTCT TACTTAGCGT TCACCTCAGC ACCTCTACTA |
| | CCTCAAGGAC |
| 401 | AAGGAGCGAT TTATAGTCTT GGTTCCGTGA TGATCGAAAA |
| | TAGTGAGGAA |
| 451 | GTGACTTTCT GTGGGAACTA CTCTTCGTGG AGTGGAGCTG |
| | CGATTTATAC |
| 501 | TCCCTACCTT TTAGGTTCTA AGGCGAGTCG TCCTTCAGTA |
| | AATCTCAGCG |
| 551 | GGAACCGCTA CCTGGTGTTT AGAGACAATG TGAGCCAAGG |
| | TTATGGCGGC |
| 601 | GCCATATCTA CCCACAATCT CACACTCACG ACTCGAGGAC |
| | CTTCGTGTTT |
| 651 | TGAAAATAAT CATGCTTATC ATGACGTGAA TAGTAATGGA |
| | GGAGCCATTG |
| 701 | CCATTGCTCC TGGAGGATCG ATCTCTATAT CCGTGAAAAG |
| | CGGAGATCTC |
| 751 | ATCTTCAAAG GAAATACAGC ATCACAAGAC GGAAATACAA |
| | TACACAACTC |
| 801 | CATCCATCTG CAATCTGGAG CACAGTTTAA GAACCTACGT |
| | GCTGTTTCAG |
| 851 | AATCCGGAGT TTATTTCTAT GATCCTATAA GCCATAGCGA |
| | GTCGCATAAA |
| 901 | ATTACAGATC TTGTAATCAA TGCTCCTGAA GGAAAGGAAA |
| | CTTATGAAGG |
| 951 | AACAATTAGC TTCTCAGGAC TATGCCTGGA TGATCATGAA |
| | GTTTGTGCGG |
| 1001 | AAAATCTTAC TTCCACAATC CTACAAGATG TCACATTAGC |
| | AGGAGGAACT |
| 1051 | CTCTCTCTAT CGGATGGGGT TACCTTGCAA CTGCATTCTT |
| | TTAAGCAGGA |
| 1101 | AGOAAGCTCT ACGCTTACTA TGTCTCCAGG AACCACTCTG |
| | CTCTGCTCAG |
| 1151 | GAGATGCTCG GGTTCAGAAT CTGCACATCC TGATTGAAGA |
| | TACCGACAAC |
| 1201 | TTTGTTCCTG TAAGGATTCG CGCCGAGGAC AAGGATGCTC |
| | TTGTCTCATT |
| 1251 | AGAAAAACTT AAAGTTGCCT TTGAGGCTTA TTGGTCCGTC |
| | TATGACTTTC |
| 1301 | CTCAATTTAA GGAAGCCTTT ACGATTCCTC TTCTTGAACT |
| | TCTAGGGCCT |
| 1351 | TCTTTTGACA GTCTTCTCCT AGGGGAGACC ACTTTGGAGA |
| | GAACCCAAGT |
| 1401 | CACAACAGAG AATGACGCCG TTCGAGGTTT CTGGTCCCTA |
| | AGCTGGGAAG |
| 1451 | AGTACCCCCC TTCTCTGGAT AAAGACAGAA GGATCACACC |
| | AACTAAGAAA |
| 1501 | ACTGTTTTCC TCACTTGGAA TCCTGAGATC ACTTCTACGC |
| | CATAA |

The PSORT algorithm predicts an outer membrane location (0.922).

The protein was expressed in E. coli and purified as a as a his-tag product and as a GST-fusion product, as shown in FIG. 26A. The recombinant GST-fusion protein was used to immunise mice, whose sera were used in a Western blot (FIG. 26B).

These experiments show that cp6735 is a surface-exposed and immunoaccessible protein, and that it is a useful immunogen. These properties are not evident from the sequence alone.

### Example 27

The following C. pneumoniae protein (PID 4376784) was expressed <SEQ ID 53; cp6784>:

| | |
|---|---|
| 1 | MNRRKARWVV ALFAMTALIS VGCCPWSQAK SRCSIDKYIP |
| | VVNRLLEVCG |
| 51 | LPEAENVEDL IESSSAWVLT PEERFSGBLV SICQVKDEHA |
| | FYNDLSLLHM |
| 101 | TQAVPSYSAT YDCAVVFGGP LPALRQELDD LVREWQRGVR |
| | FKKIVFLCGE |
| 151 | RGRYQSIEEQ EHFFDSRYNP FPTEENWESG NRVTPSSEEE |
| | IAEFVWMQML |
| 201 | LPRAWRDSTS GVRVTFLLMC PEENRVVANR KDTLLLFRSY |
| | QEAFPGRVLF |
| 251 | VSSQPFIGLD ACRVGQFFKG ESYDLAGPGF AQGVLKYHWA |
| | PRICLHTLAE |
| 301 | WLKETNGCLN ISEGCFG* |

A predicted signal peptide is highlighted.

The cp6784 nucleotide sequence <SEQ ID 54> is:

| | |
|---|---|
| 1 | ATGAATAGAA GAAAAGCAAG ATGGGTAGTG GCATTGTTCG |
| | CAATGACGGC |
| 51 | GCTCATTTCT GTTGGGTCTT GTCCTTGGTC ACAAGCGAAA |
| | TCAAGATGTT |
| 101 | CTATTGATAA GTATATTCCT GTAGTCAATC GTTTACTAGA |
| | AGTTTGTGGA |
| 151 | CTTCCTGAAG CTGAGAATGT TGAGGATTTA ATCGAGTCCT |
| | CGTCTGCTTG |
| 201 | GGTACTGACT CCTGAAGAAC GTTTTTCTGG AGAGTTAGTC |
| | TCTATCTGTC |
| 251 | AGGTTAAAGA TGAGCATGCT TTCTATAACG ATTTGTCTTT |
| | ATTACATATG |
| 301 | ACTCAGGCTG TGCCTTCGTA TTCTGCAACG TATGATTGTG |
| | CTGTAGTTTT |
| 351 | TGGCGGGCCT TTGCCAGCGC TACGTCAGCG CTTAGATTTT |
| | TTGGTGCGAG |
| 401 | AGTTGCAGCG TCGCGTGCGC TTTAAGAAAA TCGTTTTTCT |
| | ATGTGGAGAG |
| 451 | CGAGGGCGCT ATCAGTCTAT TGAAGAACAA GAGCATTTCT |
| | TTGATTCTCG |
| 501 | GTACAATCCT TTCCCTACTG AAGAGAACTG GGAATCTGGT |
| | AACCGAGTTA |
| 551 | CTCCCTCTTC TGAAGAAGAG ATTGCCAAAT TTGTTTGGAT |
| | GCAAATGCTT |
| 601 | TTACCTAGAG CATGGCGAGA TAGTACTTCA GGAGTCAGAG |
| | TGACATTTCT |
| 651 | TCTAGCAAAG CCAGAGGAAA ATCGTGTGGT TGCGAATCGT |
| | AAGGACACCT |
| 701 | TACTTTTATT CCGTTCTTAT CAAGAAGCGT TTCCGGGACG |
| | CGTGTTATTT |
| 751 | GTAAGTAGTC AACCCTTTAT CGGTTTAGAT GCTTGCAGGG |
| | TCGGGCAGTT |
| 801 | TTTCAAAGGG GAAAGCTATG ATCTGGCTGG ACCTGGATTT |
| | GCTCAAGGAG |
| 851 | TCTTGAAGTA TCATTGGGCT CCAAGGATTT GTCTACATAC |
| | TTTAGCGGAA |
| 901 | TGGTTAAAGG AAACGAACGG CTGCTTAAAT ATTTCAGAGG |
| | GTTGTTTTGG |
| 951 | ATGA |

The PSORT algorithm predicts a periplasmic location (0.894).

The protein was expressed in E. coli and purified as a his-tag product and as a GST-fusion product, as shown in FIG. 27A. The recombinant proteins were used to immunise mice, whose sera were used in a Western blot (FIG. 27B). The GST-fusion product was used for FACS analysis (FIG. 27C).

The cp6784 protein was also identified in the 2D-PAGE experiment (Cpn0498).

These experiments show that cp6784 is a surface-exposed and immunoaccessible protein, and that it is a useful immunogen. These properties are not evident from the sequence alone.

### Example 28

The following C. pneumoniae protein (PID 4376960) was expressed <SEQ ID 55; cp6960>:

| | |
|---|---|
| 1 | MNRRWNLVLA TVALLALSVAS CDVRSKDKDK DQGSVEYKD |
| | NKDTNDIELS |
| 51 | DNQKLSRTFG HLLARQLRKS EDMFFDIAEV AKGLQAELVC |
| | KSAPLTETEY |
| 101 | EEKHAEVQKL VFEKKSKENL SLAEKFLKEN SKNAGVVBVQ |
| | PSRLQYKIIK |
| 151 | EGAGKAISGK PSALIMYKGS FINGQVFSSS EGNNEPILLP |
| | LGQTIPGFAL |
| 201 | GKQGMKEGET RVLYIHPDLA YGTAGQLPPN SLLIFEINLI |
| | QASADEVAAV |
| 251 | PQEGNQGE* |

A predicted signal peptide is highlighted.

The cp6960 nucleotide sequence <SEQ ID 56> is:

| | |
|---|---|
| 1 | ATGAACAGAC GGTGGAATTT AGTTTTAGCA ACAGTAGCTC |
| | TGGCACTCTC |
| 51 | CGTCGCTTCT TGTGACGTAC GGTCTAAGGA TAAAGACAAG |
| | GATCAGGGGT |
| 101 | CGTTAGTGGA ATATAAAGAT AACAAAGATA CCAATGACAT |
| | AGAATTATCC |
| 151 | GATAATCAAA AGTTATCCAG AACATTTGGT CATTTATTAG |
| | CACOCCAATT |
| 201 | ACGCAAGTCA GAAGATATGT TTTTTGATAT TGCAGAAGTG |
| | GCTAAGGGGT |
| 251 | TGCAGGCGGA ATTGGTTTGT AAAAGTGCTC CTTTAACAGA |
| | AACAGAGTAT |
| 301 | GAAGAAAAAA TGGCTGAAGT ACAGAAGTTG GTTTTTGAAA |
| | AAAAATCAAA |
| 351 | AGAAAATCTT TCATTGGCAG AAAAATTCTT AAAAGAAAAT |
| | AGCAAGAACG |
| 401 | CTGGTGTTGT TGAAGTGCAA CCAAGTAAAT TGCAATACAA |
| | AATTATTAAA |
| 451 | GAAGGTGCAG GGAAAGCAAT TTCAGGTAAA CCTTCAGCTC |
| | TATTGCACTA |
| 501 | CAAGGGTTCC TTCATCAATG GCCAAGTATT TAGCAGTTCA |
| | GAAGGCAACA |
| 551 | ATGAGCCTAT CTTGCTTCCT CTAGGCCAAA CAATTCCTGG |
| | TTTTGCTTTA |
| 601 | GGTATGCAGG GCATGAAAGA AGGAGAAACT CGAGTTCTCT |
| | ACATCCATCC |
| 651 | TGATCTTGCT TACGGAACCG CAGGACAACT TCCTCCAAAC |
| | TCTTTATTAA |
| 701 | TTTTTGAAAT TAACTTGATT CAGGCTTCAG CAGATGAAGT |
| | TGCTGCTGTA |
| 751 | CCCCAAGAAG GAAATCAAGG TGAATGA |

The PSORT algorithm predicts periplasmic space location (0.930).

The protein was expressed in E. coli and purified as a his-tag product and as a GST-fusion product, as shown in FIG. 28A. The recombinant proteins were used to immunise mice, whose sera were used in a Western blot (FIG. 28B) and for FACS analysis (FIG. 28C).

The cp6960 protein was also identified in the 2D-PAGE experiment.

These experiments show that cp6960 is a surface-exposed and immunoaccessible protein, and that it is a useful immunogen. These properties are not evident from the sequence alone.

### Example 29

The following C. pneumoniae protein (PID 4376968) was expressed <SEQ ID 57; cp6968>:

| | |
|---|---|
| 1 | MKFLLYVPLL LVLVSTGCDA KPVSFEPFSG KLSTQRFEPQ |
| | HSAEEYSFSQG |
| 51 | QEFLKKGNFR KALLCFGIIT HHFPRDILRN QAQYLIGVCY |
| | FTQDHPDLAD |
| 101 | KAFASYLQLP DAEYSEELFQ MKYAIAQRFA QGKRKRICRL |
| | EGFPKLMNAD |
| 151 | EDALRXYDEI LTAPPSKDLG AQALYSKALL LIVKNDLTEA |
| | TKTLKKLTLQ |
| 201 | FPLHILSSEA FVRLSEIYLQ QAKKEPHNLQ YLHFAKLNEE |
| | AMKKQHPNHP |
| 251 | LNEVVSANVG AMREHYARGL YATGRFYEKK KKAEAANIYY |
| | RTAITNYPDT |
| 301 | LLVAKCQKRL DRISKHTS* |

A predicted signal peptide is highlighted.

The cp6968 nucleotide sequence <SEQ ID 58> is:

| | |
|---|---|
| 1 | ATGAAATTTC TATTATACGT TCCACTTCTT CTTGTTCTCG |
| | TATCTACGGG |
| 51 | GTGCGATGCA AAACCTGTTT CTTTTGAGCC CTPTTCAGGA |
| | AAGGTTTCCA |
| 101 | CCCAGCGTTT TGAGCCTCAG CACTCTGCTG AAGAATATTT |
| | TTCTCAGGGA |
| 151 | CAGGAATTCT TAAAAAAACG AAATTTCAGA AAAGCTTTAC |
| | TATGCTTTGG |
| 201 | AATCATTACG CATCACTTCC CTAGGGACAT CTTGCGTAAT |
| | CAAGCACAGT |
| 251 | ATCTTATAGG AGTCTGTTAC TTCACGCAGG ATCACCCAGA |
| | TTTAGCAGAC |
| 301 | AAGGCATTTG CATCTTACTT ACAACTTCCT GATGCGGAGT |
| | ACTCTGAAGA |
| 351 | GTTGTTCCAU ATGAAATATG CGATTGCTCA AAGATTTGCT |
| | CAAGGGAAGC |
| 401 | GTAAACGGAT TTGTCGATTA GAGGGCTTCC CAAAACTAAT |
| | GAATGCTGAT |
| 451 | GAAGATGCGC TACGCATTTA TGACGAGATT CTAACAGCGT |
| | TTCCTAGTAA |
| 501 | AGACTTTGGA GCTCAGGCCC TCTATAGTAA AGCTGCGTTA |
| | CTTATTGTAA |
| 551 | AAAACGATCT TACAGAAGCC ACCAAAACCT TAAAAAAACT |
| | CACGTTACAA |
| 601 | TTTCCTCTAC ATATTTTATC TTCAGGGGCC TTTGTACGTT |
| | TATCGGAAAA |
| 651 | CTATTTACAG CAAGCTAAGA AAGAGCCTCA CAATCTTCAA |
| | TATCTTCATT |
| 701 | TTGCAAAGCT TAATGAAGAG GCAATGAAAA AGCAGCATCC |
| | TAACCATCCT |
| 751 | CTGAATGAGG TTGTTTCTGC TAATGTTGGA GCTATGCGGG |
| | AACATTATGC |
| 801 | TCGAGGTTTG TATOCCACAG GTCGTTTCTA TGAGAAGAAG |
| | AAAAAAGCCG |
| 851 | AGGCTGCGAA TATCTATTAC CGCACTGCGA TTACAAACTA |
| | CCCAGACACT |
| 901 | TTATTAGTGG CTAAATGTCA AAAGCGTCTA GATAGAATAT |
| | CTAAGCATAC |
| 951 | TTCCTAA |

The PSORT algorithm predicts an inner membrane location (0.790).

The protein was expressed in E. coli and purified as a his-tag product and as a GST-fusion product, as shown in FIG. 29A. The recombinant GST-fusion was used to immunise mice, whose sera were used in a Western blot (FIG. 29B) and for FACS analysis (FIG. 29C).

This protein also showed good cross-reactivity with human sera, including sera from patients with pneumonitis.

These experiments show that cp6968 is a surface-exposed and immunoaccessible protein, and that it is a useful immunogen. These properties are not evident from the sequence alone.

### Example 30

The following C. pneumoniae protein (PID 4376998) was expressed <SEQ ID 59; cp6998>:

| | |
|---|---|
| 1 | MKKLLKSALL SAAFAGSVGS LQALLPVGNPS DPSLLIDGTI WEGAAGDPCD |
| 51 | PCATWCDAIS LRAGFYGDYV FDRILKVDAP KTFSMGAKPT GSAAANYTTA |
| 101 | VDRPNPAYNK HLHDAEWFTN AGFIALNIWD RFDVFCTLGA SNGYIRGNST |
| 151 | AFNLVGLFGV KGTTVNANEL PNVSLSNGVV ELYTDTSFSW SVGARGALWE |
| 201 | CGCATLGAEF QYAQSKPKVE BLNVICNVSQ FSVNKPKGYK GVAFPLPTDA |
| 251 | GVATATGTKS ATINYHEWQV GASLSYRLNS LVPYIGVQWS RATFDADNIR |
| 301 | IAQPKLPTAV LNLTAWNPSL LGNATALSTT DSFSDFMQIV SCQINKFKSR |
| 351 | KACGVTVGAT LVDADKWSLT AEARLINERA AHVSGQFRF* |

A predicted signal peptide is highlighted.

The cp6998 nucleotide sequence <SEQ ID 60> is:

| | |
|---|---|
| 1 | ATGAAAAAAC TCTTAAAGTC GGCGTTATTA TCCGCCGCAT TTGCTGGTTC |
| 51 | TGTTGGCTCC TTACAAGCCT TGCCTGTAGG GAACCCTTCT GATCCAAGCT |
| 101 | TATTAATTGA TGGTACAATA TGGGAAGGTG CTGCAGGAGA TCCTTGCGAT |
| 151 | CCTTGCGCTA CTTGGTGCGA CGCTATTAGC TTACGTGCTG GATTTTACGG |
| 201 | AGACTATGTT TTCGACCGTA TCTTAAAAGT AGATGCACCT AAAACATTTT |
| 251 | CTATGGGAGC CAAGCCTACT GGATCCGCTG CTGCAAACTA TACTACTGCC |
| 301 | GTAGATAGAC CTAACCCGGC CTACAATAAG CATTTACACG ATGCAGAGTG |
| 351 | GTTCACTAAT GCAGGCTTCA TTGCCTTAAA CATTTGGGAT CGCTTTGATG |
| 401 | TTTTCTGTAC TTTAGGAGCT TCTAATGGTT ACATTAGAGG AAACTCTACA |
| 451 | GCGTTCAATC TCGTTGGTTT AATCGGAGTT AAAGGTACTA CTGTAAATGC |
| 501 | AAATGAACTA CCAAACGTTT CTTTAAGTAA CGGAGTTGTT GAACTTTACA |
| 551 | CAGACACCTC TTTCTCTTGG AGCGTAGGCG CTCGTGGAGC CTTATGGGAA |
| 601 | TGCGGTTGTG CAACTTTGGG AGCTGAATTC CAATATGCAC AGTCCAAACC |
| 651 | TAAAGTTGAA GAACTTAATG TGATCTGTAA CGTATCGCAA TTCTCTGTAA |
| 701 | ACAAACCCAA GGGCTATAAA GGCGTTGCTT TCCCCTTGCC AACAGACGCT |
| 751 | GGCGTAGCAA CAGCTACTGG AACAAAGTCT GCGACCATCA ATTATCATGA |
| 801 | ATGGCAAGTA GGAGCCTCTC TATCTTACAG ACTAAACTCT TTAGTGCCAT |
| 851 | ACATTGGAGT ACAATGGTCT CGAGCAACTT TTGATGCTGA TAACATCCGC |
| 901 | ATTGCTCAGC CAAAACTACC TACAGCTGTT TTAAACTTAA CTGCATGGAA |
| 951 | CCCTTCTTTA CTAGGAAATG CCACAGCATT GTCTACTACT GATTCGTTCT |
| 1001 | CAGACTTCAT GCAAATTGTT TCCTGTCAGA TCAACAAGTT TAAATCTAGA |
| 1051 | AAAGCTTGTG GAGTTACTCT AGGAGTTACT TTAGTTGATG CTGATAAATG |
| 1101 | GTCACTTACT GCAGAAGCTC GTTTAATTAA CGAGAGAGCT CCTCACCTAT |
| 1151 | CTGGTCAGTT CAGATTCTAA |

The PSORT algorithm predicts an outer membrane location (0.707).

The protein was expressed in E. coli and purified as a GST-fusion (FIG. 30A) and as a his-tag product. The recombinant GST-fusion protein was used to immunise mice, whose sera were used in a Western blot (FIG. 30B) and for FACS analysis (FIG. 30C).

The cp6998 protein was also identified in the 2D-PAGE experiment (Cpn0695) and showed good cross-reactivity with human sera, including sera from patients with pneumonitis.

These experiments show that cp6998 is a surface-exposed and immunoaccessible protein, and that it is a useful immunogen. These properties are not evident from the sequence alone.

### Example 31

The following C. pneumoniae protein (PID 4377102) was expressed <SEQ ID 61; cp7102>:

| | |
|---|---|
| 1 | MKHTFTKRVL FFFFLVIPIP LLLNLMVVGF FSFSAAKANL VQVLHTRATN |
| 51 | LSIEFEKKLT IHKLFLDRLA NTLALKSYAS PSAEPYAQAY NEMMALSNTD |
| 101 | FSLCLIDPFD GSVRTKNPGD PFIRYLKQHP EMKKKLSAAV GKAFLLTIPG |
| 151 | KPLLHYLILV EDVASWDSTT TSGLLVSFYP MSFLQKDLFQ SLHITKGNIC |
| 201 | LVNKYGEVLF CAQDSESSFV FSLDLPNLPQ FQARSPSAIE IEKASGILGG |
| 251 | ENLITVSINK KRYLGLVLNK IPIQGTYTLS LVPVSDLIQS ALKVPLNICF |
| 301 | PYVLAFLLMW WIFSKTNTKL NKPLQELTFC MEAAWRGNHN VREEPQPYGY |
| 351 | EFNELGNTFN CTLLLLLNIS EKADIDYHSG EKLQKELGIL SSLQSALLSP |
| 401 | DFPTFPKVTF SSQHLRRRQL SGHFNGWTVQ DGGDTLLGII GLAGDIGLPS |
| 451 | YLYALSARSL FLAYASSDVS LQKISKDTAD SFSKTTEGNE AVVAMTFIKY |
| 501 | VEKDRSLELL SLSEGAPTMF LQRGESFVRL PLETHQALQP GDRLICLTGG |
| 551 | EDILKYFSQL PIEELLKDPL NPLNTENLID SLTMMLNNET EHSADGTLTI |
| 601 | LSES* |

A predicted signal peptide is highlighted.

The cp7102 nucleotide sequence <SEQ ID 62> is:

| | |
|---|---|
| 1 | ATGAAACATA CCTTTACCAA GCGTGTTCTA TTTTTTTTCT TTTTAGTGAT |
| 51 | TCCCATTCCC CTACTCCTCA ATCTTATGGT CGTAGGTTTT TTCTCADTTT |
| 101 | CTGCCGCTAA AGCAAATTTA GTACAGGTCC TCCATACCCG TGCTACGAAC |
| 151 | TTAAGTATAG AATTCGAAAA AAAACTGACG ATACACAAGC TTTTCCTCGA |
| 201 | TAGACTTGCC AACACATTAG CCTTAAAATC CTATGCATCT CCTTCTGCAG |
| 251 | AQCCCTATGC ACAGGCATAC AATGAGATGA TGGCACTCTC CAATACAGAC |
| 301 | TTTTCCTTAT GCCTTATAGA TCCCTTTGAT GGATCTGTAA GGACGAAAAA |
| 351 | TCCTGGAGAC CCTTTCATTC GCTATCTAAA ACAGCATCCT GAAATGAAGA |
| 401 | AAAAGCTATC CGCAGCTGTA GGGAAAGCCT TTTTATTGAC CATTCCAGGT |
| 451 | AAACCACTTT TACATTATCT TATTCTAGTT GAAGATGTCG CATCTTGGGA |
| 501 | TTCTACAACG ACTTCAGGAC TGCTTGTAAG TTTCTATCCC ATGTCTTTTT |
| 551 | TACAGAAAGA TTTATTCCAA TCCTTACACA TCACCAAAGG AAATATCTGC |
| 601 | CTTGTAAATA AGTATGGCGA GGTCCTCTTC TGTGCTCAGG ACAGTGAATC |
| 651 | TTCTTTTGTA TTTTCTCTAG ATCTCCCTAA TTTACCGCAA TTCCAAGCAA |
| 701 | GAAGCCCCTC TGCCATAGAA ATTGAGAAAG CTTCTGGAAT TCTTGGTGGG |
| 751 | GAGAACCTAA TCACAGTGAG TATCAACAAG AAACGCTACC TAGGATTGGT |
| 801 | ACTGAATAAA ATTCCTATCC AAGGGACCTA CACTCTATCT TTAGTTCCAG |
| 851 | TTTCTGATCT CATCCAATCC GCCTTGAAAG TTCCTCTCAA TATTTGTTTT |
| 901 | TTCTATGTAC TTGCTTTCCT CCTCATGTGG TGGATTTTCT CTAAGATCAA |
| 951 | CACCAAACTT AACAAGCCTC TTCAAGAACT GACCTTCTGT ATGGAAGCTG |
| 1001 | CCTGGCGAGG AAACCATAAC GTGAGGTTTG AACCCCAGCC TTACGGTTAT |
| 1051 | GAATTCAATG AACTAGGAAA TATTTTCAAT TGCACTCTCC TACTCTTATT |
| 1101 | GAATTCCATT GAGAAAGCAG ATATCGATTA CCATTCAGGC GAAAAATTAC |
| 1151 | AAAAAGAATT AGGGATTTPA TCTTCACTAC AAACTGCGTT ACTAAGTCCG |
| 1201 | GATTTCCCTA CGTTCCCTAA AGTTACCTTT AGTTCCCAAC ATCTCCGGAG |
| 1251 | AAGGCAACTT TCCGGTCATT TTAATGGTTG GACAGTTCAA GATGGTGGCG |
| 1301 | ATACCCTTTT AGGGATCATA GGGCTCGCTG GCGATATTGG TCTTCCTTCC |
| 1351 | TATCTCTATG CTTTATCCGC ACGGAGTCTT TTTCTTGCCT ATGCTTCCTC |
| 1401 | GGACGTTTCG TTACAAAAAA TCAGCAAGGA TACTGCCGAC AGCTTCTCAA |
| 1451 | AAACAACAGA AGGCAATGAG GCTGTAGTTG CTATGACTTT CATTAAATAT |
| 1501 | GTAGAAAAAG ATCGATCTCT AGAGCTCCTC TCGTTAAGCG AGGGAGCTCC |
| 1551 | TACCATGTTT CTACAACGAG GAGAATCTTT CGTACGTCTC CCCTTAGAGA |
| 1601 | CTCACCAAGC TCTACAGCCT GGAGATCGGT TGATCTGCCT CACTGGAGGA |
| 1651 | GAAGACATCC TCAAGTACTT TTCTCAGCTT CCTATTGAAG AGCTCTTAAA |
| 1701 | AGATCCTTTA AACCCTCTAA ATACAGAGAA TCTTATTGAT TCTCTAACCA |
| 1751 | TGATGTTAAA CAACGAAACC GAACATTCTG CAGATGGAAC TCTGACCATC |
| 1801 | CTTTCATTTT CATAA |

The PSORT algorithm predicts an inner membrane location (0.338).

The protein was expressed in E. coli and purified as a his-tag product and as a GST-fusion product. The purified GST-fusion product is shown in FIG. 31A. The recombinant GST-fusion protein was used to immunise mice, whose sera were used in a Western blot and for FACS analysis (FIG. 31B).

These experiments show that cp7102 is a surface-exposed and immunoaccessible protein, and that it is a useful immunogen. These properties are not evident from the sequence alone.

### Example 32

The following C. pneumoniae protein (PID 4377106) was expressed <SEQ ID 63; cp7106>:

| | |
|---|---|
| 1 | MKDLGTLGGT SSTAKTVSPD GKVIMGRSQI ADGSWHAFMC HTDFSSNNVL |
| 51 | FDLDNTYKTL RENGRQLNSI FNLQNMMLQR ASDHEFTEPG RSNIALGAGL |
| 101 | YVNALQNLPS NLAAQYFGIA YKIRPKYRLG VFLDHNFSSH VPNNFNVSHN |
| 151 | RLWNGAFIGW QDSDALGSSV KVSFGYGKQK ATITREQLEN TEAGSGESHF |
| 201 | BGVAAQIEGR YGKSLGGHVR VQPFLGLQFV HITRKEYTEN AVQFPVHYDP |
| 251 | IDYSTGVVYL GIGSHIALVD SLHVGTPMGM EQWFAAHTDR FSGSIASIGN |
| 301 | FVFEKLDVTH TRAFAEMRVN YELPYLQSLN LILRVNQQPL QGVMGFSSDL |
| 351 | RYALGF* |

The cp7106 nucleotide sequence <SEQ ID 64> is:

| | |
|---|---|
| 1 | ATGAAAGATT TGGGGACTCT TGGGGGTACC TCTTCTACAG CAAAAACAGT |
| 51 | GTCCCCAGAT GGTAAAGTGA TCATGGGTAG ATCACAAATT GCTGATGGCA |
| 101 | GTTGGCACGC ATTTATGTGT CATACGGATT TCTCCTCTAA TAATGTACTC |
| 151 | TTTGATCTCG ATAATACGTA TAAAACTCTA AGAGAAAATG GCCGTCAGCT |
| 201 | AAATTCCATA TTCAACCTAC AAAATATGAT GTTACAGAGA GCCTCAGATC |
| 251 | ATGAGTTCAC AGAGTTTGGA AGGAGTAACA TCGCTCTTGG TGCCGGGCTT |
| 301 | TATGTGAATG CCTTGCAGAA TCTCCCTAGC AATTTAGCAG CACAATATTT |
| 351 | TGGATTCGCA TACAAAATAC GTCCTAAATA TCGTTTGGGG GTGTTTTTGG |
| 401 | ACCATAATTT CAGCTCCCAC GTTCCTAATA ATTTTAACGT AAGCCACAAT |
| 451 | AGACTCTGGA TGGGAGCCTT TATTGGATGG CAGGATTCTG ATGCTCTAGG |
| 501 | ATCTAGTGTC AAGGTGTCTT TCGGATATGG AAAACAAAAA GCCACGATTA |
| 551 | CAAGAGAGCA ATTAGAGAAT ACAGAAGCCG GGAGTGGGGA GAGCCATTTT |
| 601 | GAAGGGGTCG CTGCTCAGAT AGAAGGGCCG TATGGTAAGA GCCTCGGAGG |
| 651 | ACATGTCAGG GTCCAGCCTT TCCTAGGACT GCAGTTTGTC CACATTACAA |
| 701 | GGAAAGAATA TACCGAAAAT GCAGTGCAAT TPCCTGTACA CTATGATCCT |
| 751 | ATAGACTATT CTACAGGTGT AGTGTATTTA GGAATTGGAT CTCATATTGC |
| 801 | ACTTGTAGAT TCTTTACATG TAGGCACACG CATGGGAATG GAGCAAAACT |
| 851 | TTGCAGCCCA TACGGACAGG TTCTCAGGAT CTATAGCGTC TATTGGAAAC |
| 901 | TTTGTGTTTG AAAAGCTTGA TGTGACTCAC ACAAGGGCAT TTGCGGAAAT |
| 951 | GCGTGTCAAC TATGAGCTTC CCTATCTACA GTCTCTGAAT CTTATTCTAC |
| 1001 | GAGTTAATCA ACAGCCTCTA CAAGGGGTTA TGGGATTTTC CAGTGATCTT |
| 1051 | AGGTATGCCT TAGGATTCTA A |

The PSORT algorithm predicts a cytoplasmic location (0.224).

The protein was expressed in E. coli and purified as a his-tag product and as a GST-fusion product. The purified GST-fusion product is shown in FIG. 32A. The recombinant GST-fusion protein was used to immunise mice, whose sera were used in a Western blot (FIG. 32B) and for FACS analysis (FIG. 32C).

This protein also showed very good cross-reactivity with human sera, including sera from patients with pneumonitis.

These experiments show that cp7106 is a surface-exposed and immunoaccessible protein, and that it is a useful immunogen. These properties are not evident from the sequence alone.

### Example 33

The following C. pneumoniae protein (PID 4377228) was expressed <SEQ ID 65; cp7228>:

| | |
|---|---|
| 1 | MTAVLILTSF PSEESARSLA RHLITERLAS CVHVFPKGTS TYLWEGKLCE |
| 51 | SEEHHIQIKS IDIRFSEICL AIQEFSGYEV PEVLIJFPIEN GDPRYLNWLT |
| 101 | ILSYPEKPPL SD* |

The cp7228 nucleotide sequence <SEQ ID 66> is:

| | |
|---|---|
| 1 | ATGACTGCTG TTCTTATTCT TACATCTTTC CCTTCGGAGG AAAGTGCTCG |
| 51 | CTCCTTAGCT AGACATCTGA TTACAGAGCG TCTTGCTTCC TGTGTGCATG |
| 101 | TATTCCCTAA AGGCACATCG ACATATCTAT GGGAAGGCAA GCTATGTGAG |
| 151 | TCTGAAGAAC ATCATATACA AATCAAATCG ATAGACATAC GCTTCTCGGA |
| 201 | AATTTGTCTT GCTATTCAGG AGTTCTCTGG CTATGAGGTT CCTGAAGTCT |
| 251 | TACTATTTCC TATTGAAAAT GGGGATCCGA GGTACTTGAA TTGGTTAACG |
| 301 | ATTCTCAGCT ATCCAGAGAA GCCTCCGCTT TCAGATTAG |

The PSORT algorithm predicts an inner membrane location (0.040).

The protein was expressed in E. coli and purified as a his-tag product and as a GST-fusion product, as shown in FIG. 33A (his-tag=left-hand arrow, GST=right-hand arrow). The proteins were used to immunise mice, whose sera were used in a Western blot (FIG. 33B) and FACS analysis.

These experiments show that cp7228 is a surface-exposed and immunoaccessible protein, and that it is a useful immunogen. These properties are not evident from the sequence alone.

### Example 34

The following C. pneumoniae protein (PID 4377170) was expressed <SEQ ID 67; cp7170>:

| | |
|---|---|
| 1 | MNSKMLKHLR LATLSFSMFF GIVSSPAVYALGAGNPAAPV LPGVNPEQTG |
| 51 | WCAPQLCNSY DLFAALAGSL KPGFYGDYVF SESAMITNVP VITSVTTSGT |
| 101 | GTTPTITSTT KNVDFDLNNS SISSSCVFAT XALQETSPAA IPLLDIAPWA |
| 151 | RVGGLKQYYR LPLNAYRDFT SNPLNAESEV TDGLIEVQSD YGIVWGLSLQ |
| 201 | KVLWKDGVSF VGVSADYRHG SSPINYXIVY NKANPEIYED ATDGNLSYKE |
| 251 | WSASIGISTY LNDYVLPYAS VSIGNTSRRA PSDSFTELEK QFTNFKFKIR |
| 301 | KITNFDRVNF CFGTTCCISN NFYYSVEGRW GYQRAINITS GLQE* |

A predicted signal peptide is highlighted.

The cp7170 nucleotide sequence <SEQ ID 68> is:

| | |
|---|---|
| 1 | ATGAATAGCA AGATGCTAAA ACATTTACGT TTAGCAACCC TTTCCTTCTC |
| 51 | TATGTTCTTC GGGATTGTAT CTTCTCCCGC AGTATATGCC CTAGGGGCTG |
| 101 | GAAACCCTGC AGCTCCAGTA CTCCCAGGTG TGAATCCTGA GCAAACGGGA |
| 151 | TGGTGTGCCT TCCAACTTTG TAATAGTTAC GATCTTTTTG CTGCTCTTGC |
| 201 | AGGAAGCCTC AAATTTGGGT TCTATGGAGA TTATGTCTTC TCAGAAAGTG |
| 251 | CCCATATTAC CAATGTCCCT GTCATTACCT CCGTTACGAC TTCAGGCACA |
| 301 | GGAACAACGC CAACCATTAC CTCTACAACT AAAAACGTAG ACTTTGATCT |
| 351 | TAACAACAGC TCCATCAGCT CGAGCTGTGT TTTTGCAACC ATAGCTCTAC |
| 401 | AGGAAACATC CCCAGCTGCC ATTCCCCTTT TAGATATAGC CTTCACTGCA |
| 451 | CGTGTCGGAG GACTTAAGCA GTACTACCGC CTCCCTCTCA ATGCTTACAG |
| 501 | AGACTTCACT TCAAATCCTT TAAATGCAGA ATCTGAAGTT ACCCATGGTC |
| 551 | TCATTGAAGT CCAGTCAGAC TATGGAATTC TCTGGGCTCT GAGGTTACAA |
| 601 | AAAGTATTGT GGAAAGATGG AGTGTCTTTT GTAGGGGTGA GCGCTGACTA |
| 651 | CCGTCACGGT TCCAGTCCCA TCAACTATAT CATCGTTTAC AACAAGGCCA |
| 701 | ACCCCGAGAT CTATTTCGAT GCTACTGATG GAAACCTAAG CTATAAAGAA |
| 751 | TGGTCTGCAA GCATCGGCAT CTCTACGTAT CTTAATGACT ATGTGCTTCC |
| 801 | CTATGCATCC GTATCTATAG GAAATACTTC AAGAAAAGCT CCTTCTGATA |
| 851 | GCTTCACAGA ACTCGAAAAC CAATTTACGA ATTTTAAATT TAAAATTCGT |
| 901 | AAAATCACAA ACTTCGACAG AGTAAACTTC TGCTTCGGAA CTACCTGCTG |
| 951 | CATCDCAAAT AACTTCTACT ATAGTGTAGA AGGCCGTTGG GGATATCAGC |
| 1001 | GTGCTATCAA CATTACGTCA GGTCTGCAGT TTTAG |

The PSORT algorithm predicts a bacterial outer membrane location (0.936).

The protein was expressed in E. coli and purified as a his-tag product and as a GST-fusion product. The purified GST-fusion product is shown in FIG. 34A. The GST-fusion protein was used to immunise mice, whose sera were used in a Western blot (34B) and for FACS analysis (34C).

The cp7170 protein was also identified in the 2D-PAGE experiment (Cpn0854).

These experiments show that cp7170 is a surface-exposed and immunoaccessible protein, and that it is a useful immunogen. These properties are not evident from the sequence alone.

### Example 35

The following C. pneumoniae protein (PID 4377072) was expressed <SEQ ID 69; cp7072>:

| | |
|---|---|
| 1 | MDIKKLFCLF LCSSLIAMSPIYGKTGDYEK LTLTGINIID RNGLSETICS |
| 51 | KEKLKKYThV DFLAPQPYQK VMRMYKNKRG DNVSCLTAYH TNGQIKQYLE |
| 101 | CLNNRAYGRY REWHVNGNIK IQAEVIGGIA DLIHPSAEGW LFDQTTFAYN |
| 151 | DEGXLEAAIV YEKGLLEGSS VYYHTNGNIW KECPYHKGVP QGKFLTYTSS |
| 201 | GKLLKEQNYQ QGKRHGLSIR YSEDSEEDVL AWEEYHEGRL LKAEYLDPQT |
| 251 | HEIYATIHEG NGIQAIYGKY AVIETRAFYR GEPYGKVTRF DNSGTQIVQT |
| 301 | YNLLQGAKHG EEDTFYPETG KPKLLLNWHE GILNGIVKTW YPGGTLESCK |
| 351 | ELVNNKKSGL LTXYYPEGQI HATEEYDNDL LIKGEYFRPG DRHPYSKIDR |
| 401 | GCGTAVFFSS AGTITKKIPY QDGKPLIN* |

A predicted signal peptide is highlighted.

The cp7072 nucleotide sequence <SEQ ID 70 is:

| | |
|---|---|
| 1 | ATGGATATAA AAAAACTCTT TTGCTTATTT CTATGTTCTT CTCTAATTGC |
| 51 | CATGAGTCCC ATTTATGGGA AAACAGGTGA CTATGAGAAA CTCACCCTTA |
| 101 | CAGGGAPCAA TATCATTGAT AGAAACGGCC TGTCAGAAAC TATTTGCTCT |
| 151 | AAAGAGAAGC TAAAGAAATA CACCAAGGTA GACTTTCTTG CTCCCCAGCC |
| 201 | CTATCAAAAG GTCATGAGGA TGTATAAAAA CAAACGCGGA GATAACGTTT |
| 251 | CTTGTTTAAC AGCCTATCAC ACTAACGGGC AAATTAAGCA GTACCTGGAG |
| 301 | TGTCTCAATA ATCGTGCTTA TGGAAGATAT CGTGAATGGC ACGTCAACGG |
| 351 | GAATATCAAA ATCCAAGCTG AGGTTATCGG AGGTATTGCG GATCTTCATC |
| 401 | CCTCAGCAGA GTCTGGCTGG CTATTTGATC AAACTACATT TGCCTATAAT |
| 451 | GATGAAGGTA TCTTAGAAGC CGCTATCGTC TATGAAAAAG GGCTGCTCGA |
| 501 | AGGATCTTCG GTGTATTACC ATACTAATGG GAATATTTGG AAAGAGTGTC |
| 551 | CCTATCATAA GGGAGTTCCT CAAGGTAAAT TCCTGACATA CACATCTTCG |
| 601 | GGGAAACTGC TCAAAGAACA GAATTACCAA CAAGGCAAAA GACACGGTCT |
| 651 | TTCGATTCGC TACAGCGAAG ATTCCGAAGA AGATGTTTTA GCCTGGGAAG |
| 701 | AATATCATGA GGGACGACTC CTAAAAGCAG AGTACTTAGA TCCTCAAACT |
| 751 | CACGAAATCT ATGCGACTAT ACACGAAGGG AACGGCATTC AAGCAATCTA |
| 801 | CGGCAAGTAT GCCGTTATAG AAACTAGGGC ATTTTACCGA GGGGAACCTT |
| 851 | ATGGAAAAGT TACCAGATTC GACAACTCCG GAACACAGAT TGTCCAAACG |
| 901 | TATAACCTTT TGCAAGGCGC GAAGCACGGA GAAGAATTTT TCTTTTATCC |
| 951 | TGAGACAGGG AAACCCAAGC TGCTTCTTAA TTGGCATGAA GGAATTTTAA |
| 1001 | ATGGGATAGT AAAAACTTGG TATCCCGGAG GAACCTTAGA AAGTTGTAAA |
| 1051 | GAACTCGTAA ATAACAAAAA ATCCGGGTTA CTGACCATTT ACTACCCTGA |
| 1101 | AGGACAGATC ATGGCGACCG AAGAGTATGA TAATGATCTT CTAATTAAAG |
| 1151 | GAGAGTACTT CCGCCCTGGA GACCGTCATC CCTACTCTAA AATAGATCGT |
| 1201 | GGTTGTGGGA CTGCAGTATT TTTCTCGTCG GCGGGAACTA TTACTAAAAA |
| 1251 | AATCCCCTAT CAGGACGGCA AACCTTTGCT CAACTAG |

The PSORT algorithm predicts a periplasmic location (0.688).

The protein was expressed in E. coli and purified as a his-tag product (FIG. 35A) and as a GST-fusion product (FIG. 35B). The recombinant his-tag protein was used to immunise mice, whose sera were used in a Western blot (FIG. 35C) and for FACS analysis.

These experiments show that cp7072 is a useful immunogen. These properties are not evident from the sequence alone.

### Example 36

The following C. pneumoniae protein (PID 4376879) was expressed <SEQ ID 71; cp6879>:

| | |
|---|---|
| 1 | MATPAQKSPT FQDPSFVREL GSNHPVFSPL TLEERGEMAI ARVQQCGWNH |
| 51 | TIVKVSLIIL ALLTILGGGL LVGLLPAVPH FIGTGLIALG AVIFALALIL |
| 101 | CLYDSQGLPE ELPPVPEPQQ IQIEDLRNET REVLEGTLLE VLLKDRDAIW |
| 151 | PAVPQVVVDC EKRLGMLDRK LRREEEILYR STAHLKDEER YEFLLELLEM |
| 201 | RSLVADRLEE NRRSYERFVQ GIMTVRSEEG EKEISRLQDL ISLQQQTVQD |
| 251 | LRSRIDDEQK RCWTALQRIN QSQKDIQRAH DREASQRACE GTEMDCAERQ |
| 301 | QLEKDLRRQL KSMQEWIEMR GTIHQQEKAW RKQNAKLERL QEDLRLTGIA |
| 351 | PDEQSLEYRE YKEKYLSQXL DMQKILQEVN AEKSEKACLE SLVHDYEKQL |
| 401 | EQKJWILKKA AAVWEEELGK QQQEDYEQTQ EIEPLSTFIL EYQDSLREAE |
| 451 | KVEKDFQELQ QRYSRLQEEK QVKEKILEES MNHFADLFEK AQKENMAYKK |
| 501 | KLADLEGAAA PTEIGEDDDW VLTDSASLSQ KKIRELVEEN QELLKALAFK |
| 551 | SNELTQLVAD AVEAEKEISK LREHIEEQKE GLRALDKMHA QAIKDCEAAQ |
| 601 | RKCCDLESLL SPVREDAGMR FELEVELQRL QEENAQLRAE VERLEQEQFQ |
| 651 | G* |

The cp6879 nucleotide sequence <SEQ ID 72> is:

| | |
|---|---|
| 1 | ATGGCAACAC CCGCTCAAAA ATCCCCTACA TTTCAAGATC CTAGTTTTGT |
| 51 | AAGAGAGCTA GGCAGTAACC ACCCTGTCTT TTCCCCGCTA ACGCTTGAGG |
| 101 | AAAGAGGGGA GATGGCAATA GCTCGAGTCC AGCAGTGTGG ATGGAATCAT |
| 151 | ACAATTGTTA AGGTAAGTCT TATTATTCTT GCTCTTCTTA CTATPTTAGG |
| 201 | GGGAGGATTA CTCGTAGGAT TGCTGCCAGC AGTTCCTATG TTTATTGGAA |
| 251 | CAGGTCTGAT TGCTTTGGGA GCCGTTATAT TTGCTTTGGC TTTGATTTTA |
| 301 | TGTCTTTATG ATTCTCAGGG CCTTCCTGAG GAACTCCCTC CGGTTCCTGA |
| 351 | ACCACAACAA ATTCAGATTG AAGATTTAAG AAACGAGACC AGAGAAGTTC |
| 401 | TTGAAGGGAC TCTTTTAGAG GTTCTCTTAA AGGATAGAGA CGCTAAGGAC |
| 451 | CCTGCGGTGC CCCAGGTGGT TGTAGACTGT GAAAAGCGTC TTGGAATGTT |
| 501 | GGATCGTAAG CTGCGACGTG AAGAGGAGAT TCTGTATCGC TCGACGGCCC |
| 551 | ATCTTAAAGA CGAGGAAAGG TATGAGTTCT TGCTGGAGCT CTTGGAAATG |
| 601 | CGTAGTCTGG TTGCCGATCG GCTAGAATTT AACCGTAGAA GTTATGAGCG |
| 651 | ATTTGTTCAA GGAATTATGA CAGTTAGATC AGAGGAGGGG GAAAAAGAGA |
| 701 | TTTCTCGTCT ACAAGATCTA ATCAGTTTGC AGCAGCAGAC GGTGCAAGAT |
| 751 | TTAAGGAGTC GGATCGATGA CGAGCAGAAG AGATGCTGGA CGGCTTTACA |
| 801 | ACGTATTAAC CAATCTCAGA AGGATATACA ACCGGCTCAT GATCGCGAGG |
| 851 | CTTCGCAGCG TGCCTGTGAG GGCACAGAGA TGGATTGTGC AGAACGCCAG |
| 901 | CAACTGGAGA AGGATTTAAG GAGACAGCTG AAATCTATGC AGGAGTGGAT |
| 951 | TGAGATGAGG GGCACAATCC ATCAACAAGA GAAGGCTTGG CGTAAGCAGA |
| 1001 | ATGCCAAATT AGAAAGATTA CAAGAGTATC TGAGACTTAC TGGGATTGCT |
| 1051 | TTTGACGAAC AATCTCTGTT CTATCGCGAA TATAAAGAGA AATATCTGAG |
| 1101 | TCAGAAACTA GATATGCAAA AGATTTTACA GGAAGTCAAC GCAGAGAAAA |
| 1151 | GTGAGAAGGC TTGCTTAGAG AGTCTGGTCC ATGACTATGA GAAGCAGCTC |
| 1201 | GAACAAAAAG ATGCTAATCT GAAGAAAGCA GCAGCTGTTT GGGAAGAAGA |
| 1251 | ATTAGGGAAG CAGCAACAGG AAGACTACGA ACAAACCCAA GAAATTAGAC |
| 1301 | GTCTGAGTAC ATTCATTCTT GAGTACCAGG ACAGTCTGCG TGAGGCAGAA |
| 1351 | AAAGTTGAGA AAGATTTCCA AGAGCTACAA CAAAGGTATA GCCGTCTTCA |
| 1401 | AGAGGAGAAA CAGGTAAAAG AAAAAATCTT AGAAGAAAGT ATGAATCATT |
| 1451 | TTGCCGATCT CTTTGAGAAG GCTCAAAAGG AAAACATGGC CTACAAGAAG |
| 1501 | AAGTTAGCGG ATTTAGAGGG TGCCGCTGCT CCTACTGAGA TCGGTGAGGA |
| 1551 | CGATGACTGG GTACTCACAG ATTCTGCTTC TCTCAGCCAG AAGAAGATCC |
| 1601 | GCGAACTCGT GGAAGAGAAT CAAGAACTCC TGAAAGCACT TGCATTTAAA |
| 1651 | TCTAACGAAT TGACTCAACT GGTTGCCGAT GCTGTAGAAG CTGAAAAAGA |
| 1701 | AATCAGCAAG CTTCGAGAAC ACATAGAAGA GCAGAAAGAA GGATTACGAG |
| 1751 | CTCTTGATAA GATGCATGCA CAAGCGATCA AAGATTGCGA AGCTGCTCAG |
| 1801 | AGAAAATGCT GTGACCTTGA GAGCCTTCTC TCTCCTGTTC GAGAAGATGC |
| 1851 | TGGAATGAGA TTTGAGCTAG AGGTCGAGCT TCAAAGATTG CAAGAAGAAA |
| 1901 | ATGCACAGCT TAGAGCGGAG GTTGAAAGAC TAGAGCAAGA GCAATTTCAA |
| 1951 | GGATAA |

The PSORT algorithm predicts an inner membrane location (0.646).

The protein was expressed in E. coli and purified as a his-tag product and as a GST-fusion product. The purified GST-fusion product is shown in FIG. 36A. The recombinant GST-fusion protein was used to immunise mice, whose sera were used in a Western blot (FIG. 36B) and for FACS analysis.

These experiments show that cp6879 is useful immunogen. These properties are not evident from the sequence alone.

### Example 37

The following C. pneumoniae protein (PID 4376767) was expressed <SEQ ID 73; cp6767>:

| | |
|---|---|
| 1 | MIKQIGRFFR AFIFIMPLSL TSCESKIDRN RIWIVGTNAT YPPFEYVDAQ |
| 51 | GEVVGFDIDL AKAISEKLGK QLEVREFAFD ALILNLKKHR IDAILAGMSI |
| 101 | TPSRQKEIAL LPYYGDBVQE LMVVSKRSLE TPVLPLTQYS SVAVQTGTFQ |
| 151 | EHYLLSQPGI CVRSFDSTLE VIMEVRYGKS PVAVLEPSVG RVVLKDFPNL |
| 201 | VATPLELPPE CWVLGCGLGV AKDRPEEIQT IQQAITDLKS EGVIQSLTKK |
| 251 | WQLSEVAYR* |

The cp6767 nucleotide sequence <SEQ ID 74> is:

| | |
|---|---|
| 1 | ATGATAAAAC AAATAGGCCG TTTTTTTAGA GCATTTATTT TTATAATGCC |
| 51 | TTTATCTTTA ACAAGTTGTG AGTCTAAAAT CGATCGAAAT CGCATCTGGA |
| 101 | TTGTAGGTAC GAATGCTACA TATCCTCCTT TTGAGTATGT GGATGCTCAG |
| 151 | GGGGAAGTTG TAGGTTTCGA TATAGATTTG GCAAAGGCAA TTAGTGAAAA |
| 201 | ACTTGGCAAG CAATTGGAAG TTAGAGAATT CGCTTTCGAT GCTTTAATTT |
| 251 | TAAATTTAAA AAAACATCGT ATCGATGCAA TTTTAGCAGG AATGTCCATT |
| 301 | ACTCCTTCGC GTCAGAAGGA AATCGCCCTG CTTCCCTATT ATGGCGATGA |
| 351 | GGTTCAAGAG CTGATGGTGG TTTCTAAGCG GTCTTTAGAG ACCCCTGTGC |
| 401 | TTCCCCTAAC ACAGTATTCT TCTGTTGCTG TTCAGACAGG AACGTTTCAG |
| 451 | GAGCATTATC TTTTATCTCA GCCCGGAATT TGTGTCCGTT CTTTTGATAG |
| 501 | CACCTTGGAG GTGATTATGG AAGTTCGTTA TGGGAAATCT CCGGTTGCCG |
| 551 | TTCTAGAACC CTCGGTAGGA CGTGTCGTTC TTAAAGACTT CCCTAATCTT |
| 601 | GTTGCAACAA GATTAGAGCT CCCTCCTGAA TGTTGGGTGT TGGGCTGTGG |
| 651 | TCTCGGCGTA GCTAAAGATC GTCCTGAAGA AATACAAACG ATTCAACAAG |
| 701 | CGATTACAGA TTTAAAGAGC GAAGGGGTGA TTCAATCTTT AACCAAGAAA |
| 751 | TGGCAACTTT CTGAAGTTGC TTACGAATAG |

The PSORT algorithm predicts an inner membrane location (0.083).

The protein was expressed in E. coli and purified as a his-tag product and as a GST-fusion product. The purified his-tag product is shown in FIG. 37A. The recombinant his-tag protein was used to immunise mice, whose sera were used in a Western blot (FIG. 37B) and for FACS analysis (FIG. 37C). The GST-fusion was also used in a Western blot (FIG. 37D).

The cp6767 protein was also identified in the 2D-PAGE experiment and showed good cross-reactivity with human sera, including sera from patients with pneumonitis.

These experiments show that cp6767 is a useful immunogen. These properties are not evident from the sequence alone.

### Example 38

The following C. pneumoniae protein (PID 4376717) was expressed <SEQ ID 75; cp6717>:

| | |
|---|---|
| 1 | MMSRLRFRLA ALGIFFILLV PNSVSAKTIV ASDKEKVGVL VYDNSVEAFQ |
| 51 | QILDCIDHAN FYVELCPCMT GGRTLKEMVD HLEARMDLVP ELCSYIIIQP |
| 101 | TFTDAEDQKL LKALKERHPN RFFYVFTGCP PSTSILAPNV IEMHIKLSII |
| 151 | DGKYCILGGT NFEEFMCTPG DEVPEKVDNP RLFVSGVRRP LAFRDQDIML |
| 201 | RSTAFGLQLR EEYHXQPAMW DYYAHHMWFI DNPEQFAGAC PPLTLEQAEE |
| 251 | TVFPGFDKHE DLVLVDSSKI RIVLGGPHDK QPNPVTQEYL KLIQGARSSV |
| 301 | KLAHMYFIPK DELLNALVDV SHNHGVHLSL ITNGCHELSP AITGPYAWGN |
| 351 | RINYFALLYG KRYPLWKKWF CEKLKPYERV SIYEFAIWET QLHKKCMIID |
| 401 | DEIFVIGSYN FGKKSDAFDY ESIVVIESPE VAAKANKVFN KDIGLSIPVS |
| 451 | HGDIPSWYPH SVHHTLGHLQ LTYMPA* |

A predicted signal peptide is highlighted.

The cp6717 nucleotide sequence <SEQ ID 76> is:

| | |
|---|---|
| 1 | ATGATGAGTC GGTTGCGTTT TCGCTTGGCA GCTCTTGGAA TATTTTTTAT |
| 51 | TTTGCTGGTT CCTAATTCTG TTTCAGCAAA GACAATCGTA GCTTCAGACA |
| 101 | AGGAGAAGGT TGGAGTTCTT GTTTATGACA ATAGTGTAGA GGCCTTTCAA |
| 151 | CAGATATTGG ATTGCATAGA TCATGCAAAT TTTTATGTAG AACTGTCTCC |
| 201 | CTGCATGACA GGAGGCCGAA CGCTTAAAGA GATGGTAGAT CACCTCGAGG |
| 251 | CTCGTATGGA TCTGGTTCCA GAGCTCTGTA GCTATATCAT TATCCAACCC |
| 301 | ACGTTTACCG ATGCTGAAGA CCAAAAATTA CTCAAAGCTC TCAAAGAACG |
| 351 | TCATCCCAAC CGGTTTTTCT ACGTTTTTAC AGGGTGCCCA CCCTCAACAA |
| 401 | GCATCCTCGC TCCTAATGTC ATTGAAATGC ATATCAAACT TTCTATCATC |
| 451 | GATGGGAAAT ATTGTATTTT AGGTGGTACC AATTTTGAAG AGTTTATGTG |
| 501 | CACTCCAGGG GATGAGGTTC CTGAGAAAGT GGATAACCCA CGTTTATTTG |
| 551 | TCAGTGGAGT GCGTCGGCCC CTAGCATTTC GTGATCAGGA TATCATGTTG |
| 601 | CGTTCTACAG CATTCGGTTT GCAGCTCAGA GAAGAATATC ATAAGCAATT |
| 651 | TGCTATGTGG GACTACTATG CACATCATAT GTGGTTCATT GATAATCCTG |
| 701 | AACAGTTTGC AGGCGCCTGT CCTCCACTGA CTTTAGAACA AGCCGAGGAG |
| 751 | ACAGTATTTC CTGGATTTGA CAAACATGAA GATCTTGTTC TTGTCGACTC |
| 801 | TTCCAAGATC AGGATAGTTT TAGGTGGTCC CCACGATAAG CAACCCAATC |
| 851 | CTGTGACTCA AGAATATTTG AAACTTATCC AGGGAGCTAG ATCTTCTGTG |
| 901 | AAGCTTGCTC ACATGTATTT CATCCCTAAG GACGAGCTTT TAAATGCTCT |
| 951 | TGTCGACGTT TCTCATAATC ACGGTGTTCA TCTGAGTTTA ATTACGAACG |
| 1001 | GCTGTCATGA ATTAAGTCCT GCAATTACAG GACCCTATGC TTGGGGAAAC |
| 1051 | CGTATTAACT ATTTCGCCTT GCTCTATGGG AAACGGTATC CTCTTTGGAA |
| 1101 | AAAATGGTTT TGCGAAAAGC TAAAACCTTA TGAGCGGGTT TCTATTTATG |
| 1151 | AGTTTGCTAT TTGGGAAACG CAGTTGCACA AGAAGTGTAT GATTATCGAT |
| 1201 | GATGAAATTT TTGTGATCGG AAGTTATAAT TTTGGAAAGA AAAGTGATGC |
| 1251 | CTTTGATTAC GAAAGTATTG TAGTTATCGA ATCTCCAGAA GTCGCTGCAA |
| 1301 | AAGCTAACAA AGTCTTCAAT AAAGATATCG GATTGTCGAT TCCTGTAAGT |
| 1351 | CATGGCGACA TTTTCTCTTG GTATTTCCAT TCCGTACACC ACACTTTGGG |
| 1401 | ACATTTGCAG CTGACCTATA TGCCAGCCTA G |

The PSORT algorithm predicts a periplasmic location (0.939).

The protein was expressed in E. coli and purified as a GST-fusion (FIG. 38A), as a his-tagged protein, and as a GST/his fusion product. The proteins were used to immunise mice, whose sera were used in a Western blot (FIG. 38B) and for FACS analysis.

These experiments show that cp6717 is a useful immunogen. These properties are not evident from the sequence alone.

### Example 39

The following C. pneumoniae protein (PID 4376577) was expressed <SEQ ID 77; cp6577>:

| | |
|---|---|
| 1 | MKKLLFSTFL LVIGSTSAAH ANLGYVNLKR CLEESDLGKK ETEELEAMKQ |
| 51 | QFVKNAEKIE EELTSIYNKL QDEDYMHSLS DSASEELPKK FEDLSGEYNA |
| 101 | YQSQYYQSIN QSNVKRIQKL IQEVKIAAES VRSKEKLEAI LNEEAVLAIA |
| 151 | PGTDKTTEII AILNESFKKQ N* |

A predicted signal peptide is highlighted.

The cp6577 nucleotide sequence <SEQ ID 78> is:

| | |
|---|---|
| 1 | ATGAAAAAAT TATTATTTTC TACATTTCTT CTTGTTTTAG GATCAACAAG |
| 51 | CGCAGCTCAT GCAAATTTAG GCTATGTTAA TTTAAAGCGA TGTCTTGAAG |
| 101 | AATCCGATCT AGGTAAAAAG GAAACTGAAG AATTGGAAGC TATGAAACAG |
| 151 | CAGTTTGTAA AAAATGCTGA GAAAATAGAA GAAGAACTCA CTTCTATTTA |
| 201 | TAATAAGTTG CAAGATGAAG ATTACATCGA AAGCCTATCG GATTCTGCCT |
| 251 | CTGAAGAGTT GCGAAAGAAA TTCGAAGATC TTTCAGGAGA GTACAAPGCG |
| 301 | TACCAGTCTC AGTACTATCA ATCTATCAAT CAAAGTAATG TAAAACGCAT |
| 351 | TCAAAAACTC ATTCAAGAAG TAAAAATAGC TGCAGAATCA GTGCGGTCCA |
| 401 | AAGAAAAACT AGAAGCTATC CTTAATGAAG AAGCTGTCTT AGCAATAGCA |
| 451 | CCTCGGACTG ATAAAACAAC CGAAATTATT GCTATTCTTA ACGAATCTTT |
| 501 | CAAAAAACAA AACTAG |

The PSORT algorithm predicts a periplasmic space location (0.932).

The protein was expressed in E. coli and purified as a his-tag product (FIG. 39A) and as a GST-fusion product (FIG. 39B). The recombinant GST-fusion protein was used to immunise mice, whose sera were used in a Western blot (FIG. 39C) and for FACS analysis.

The cp6577 protein was also identified in the 2D-PAGE experiment.

These experiments show that cp6577 is a useful immunogen. These properties are not evident from the sequence alone.

### Example 40

The following C. pneumoniae protein (PID 4376446) was expressed <SEQ ID 79; cp6446>:

| | |
|---|---|
| 1 | MKQPMSLIFS SVCLGLGLGS LSSCNQKPSW NYHNTSTSEE |
| | FFVHGNKSVS |
| 51 | QLPHYPSAFR TTQIFSEEHN DPYVVAKTDE ESRKIWREIH |
| | KNLKIKGSYI |
| 101 | PISTYGSIMH PKSAALTLKT YRPHPIWING YERSFNIDTG |
| | KYLKNGSRRR |
| 151 | TSHDGPKNRA VLNLIKSSGR RCNAIGLEMT EEDFVIARRR |
| | EGVYSLYPVE |
| 201 | VCSYPQGNPF VIAYAWIADE SACSKEVLPV KGYYSLVWES |
| | VSSSDSLNAF |
| 251 | GDSFAEDYLR STFLANGTSI LCVHESYKKV PPQP* |

A predicted signal peptide is highlighted.

The cp6446 nucleotide sequence <SEQ ID 80> is:

| | |
|---|---|
| 1 | ATGAAACAGC CCATGTCTCT TATCTTTTCA AGTGTATGTT |
| | TAGGATTAAG |
| 51 | TCTTGGATCT CTTTCCTCCT GTAATCAAAA GCCCTCTTGG |
| | AATTATCACA |
| 101 | ACACTTCAAC GAGCGAAGAA TTCTTTGTTC ATGGAAATAA |
| | GAGTGTTTCG |
| 151 | CAACTGCCTC ATTATCCTTC TGCATTTCGT ACGACTCAAA |
| | TCTTTTCTGA |
| 201 | AGAGCACAAT GATCCTTATG TCGTAGCTAA GACTGATGAA |
| | GAGTCTCGTA |
| 251 | AAATTTGGAG AGAAATCCAT AAAAATCTCA AAATCAAAGG |
| | TTCTTACATT |
| 301 | CCCATATCGA CTTATGGAAG TCTGATGCAC CCAAAATCAG |
| | CAGCTCTTAC |
| 351 | ATTAAAAACG TATCGTCCAC ATCCTATTTG GATAAATGGA |
| | TACGAGCGTT |
| 401 | CTTTTAATAT AGACACAGGA AAGTACTTAA AAAACGGAAG |
| | TCGCCGTAGA |
| 451 | ACTTCTCACG ATGGTCCGAA AAATCGAGCT GTACTGAAPC |
| | TCATTAAATC |
| 501 | TTCGGGACGA CGCTGTAATG CTATAGGCCT TGAGATGACA |
| | GAAGAAGACT |
| 551 | TTGTAATAGC TAGAAGGCGA GAAGGTGTTT ATAGCCTGTA |
| | TCCCGTTGAA |
| 601 | GTGTGCTCGT ATCCTCAGGG GAATCCTTTT GTCATTGCTT |
| | ATGCCTGGAT |
| 651 | TGCAGATGAG AGTGCTTGCT CAAAAGAGGT CCTACCTGTA |
| | AAAGGGTACT |
| 701 | ATTCTTTAGT CTGCGAAAGC GTTTCTTCCT CTGATTCTCT |
| | GAATGCTTTT |
| 751 | GGAGATTCCT TTGCAGAGGA CTACCTCAGA AGCACGTTTT |
| | TAGCAAACGG |
| 801 | AACTTCTATA CTCTGTGTTC ATGAAAGCTA TAAGAAAGTT |
| | CCTCCTCAGC |
| 851 | CCTAA |

The PSORT algorithm predicts an inner membrane location (0.177).

The protein was expressed in E. coli and purified as a his-tag product and a GST-fusion product. The GST-fusion product is shown in FIG. 40A. The recombinant his-tag protein was used to immunise mice, whose sera were used in a Western blot (FIG. 40B) and for FACS analysis.

These experiments show that cp6446 is a useful immunogen. These properties are not evident from the sequence alone.

### Example 41

The following C. pneumoniae protein (PID 4377108) was expressed <SEQ ID 81; cp7108>:

| | |
|---|---|
| 1 | MKQPMSLIFS SVCLGLGLGS LSSCNQKPSW NYHNTSTSEE |
| | FFVHGNKSVS |
| 51 | TFTDLELLSK EGWSEAHAVS GNGSRIVGAS GAGQGSVTAV |
| | IWESHLIKHL |
| 101 | GTLGGEASSA EGISKDGEVV VGWSDTREGY THAFVFDGRD |
| | MKDLGTLGAT |
| 151 | YSVARGVSGD GSIIVGVSAT ARGEDYGWQV GVKWEKGKIK |
| | QLKLLPQGLW |
| 201 | SEANAISEDG TVIVGRGEIS RNHIVAVKWN KNAVYSLGTL |
| | GGSVASAEAI |
| 251 | SANGKVIVGW STTNNGETHA FMHKDETMHD LGTLGGGFSV |
| | ATGVSADGRA |
| 301 | IVGFSAVKTG EIHAFYYAEG EMEDLTTLGG EEARVPDISS |
| | EGNDIIGSIK |
| 351 | TDAGAERAYL FHIHK* |

A predicted signal peptide is highlighted.

The cp7108 nucleotide sequence <SEQ ID 82> is:

| | |
|---|---|
| 1 | ATGAGTAAGA AGATAAAGGT TCTAGGTCAT TTGACGCTCT |
| | GCACTCTGTT |
| 51 | TAGAGGAGTG CTGTGTGCAG CGGCCCTTTC CAACATAGGA |
| | TATGCGAGTA |
| 101 | CTTCTCAGGA ATCACCATAT CAGAAGTCTA TAGAAGACTG |
| | GAAAGGGTAT |
| 151 | ACCTTTACAG ATCTTGAGTT ACTGAGTAAG GAAGGGTGGT |
| | CTGAAGCTCA |
| 201 | TGCAGTTTCT GGAAATGGCA GTAGAATTGT AGGAGCTTCG |
| | GGAGCTGGCC |
| 251 | AAGGTAGTGT GACTGCTGTC ATATGGGAAA GTCACCTGAT |
| | AAAACATCTC |
| 301 | GGCACTTTAG GTGGCGAGGC TTCATCTGCA GAGGGAATTT |
| | CAAAGGATGC |
| 351 | AGAGGTGGTC GTTGGGTGGT CAGATACTAG AGAGGGATAT |
| | ACTCATGCCT |
| 401 | TTGTCTTCGA CGGTAGAGAT ATGAAAGATC TCGGTACTCT |
| | AGGAGCTACC |
| 451 | TATTCTGTAG CAAGGGGTGT TTCTGGAGAT GGTAGTATCA |
| | TCGTAGGAGT |
| 501 | CTCTGCAACT GCTCGTGGAG AGGATTACGG ATGGCAAGTT |
| | GGTGTCAAGT |
| 551 | GGGAAAAAGG GAAAATCAAA CAATTGAAGT TGTTGCCTCA |
| | AGGTCTCTGG |
| 601 | TCTGAGGCGA ATGCAATCTC TGAGGATGGT ACGGTGATTG |
| | TCGGGAGAGG |
| 651 | GGAAATCTCT CGCAATCACA TCGTTGCTGT AAAATGGAAT |
| | AAAAATGCTG |
| 701 | TGTATAGTTT GGGGACTCTC GGAGGTAGTG TCGCTTCAGC |
| | AGAGGCTATA |
| 751 | TCGGCAAATG GGAAAGTAAT TGTAGGATGG TCCACGACTA |
| | ATAATGGTGA |
| 801 | GACTCATGCC TTTATGCTCA AAGATGAGAC AATGCACGAT |
| | CTCGGCACTC |
| 851 | TAGGAGGAGG TTTTTCTGTC GCAACTGGAG TTTCTGCTGA |
| | TGGGAGAGCC |
| 901 | ATCGTAGGAT TTTCAGCAGT GAAGACCGGA GAAATTCATG |
| | CTTTTTACTA |
| 951 | TGCAGAAGGA GAAATGGAGG ATTTAACAAC TTTGGGAGGG |
| | GAAGAAGCTC |
| 1001 | GAGTGTTCGA CATATCTAGC GAAGGAAACG ATATCATTGG |
| | CTCTATAAAA |
| 1051 | ACTGACGCTG GAGCTGAACG CGCCTATCTG TTCCATATAC |
| | ATAAATAA |

The PSORT algorithm predicts an outer membrane location (0.921).

The protein was expressed in E. coli and purified as a GST-fusion product, as shown in FIG. 41A. The recombinant protein was used to immunise mice, whose sera were used in a Western blot (FIG. 41B) and for FACS analysis FIG. 41C). A his-tagged protein was also expressed.

The cp7108 protein was also identified in the 2D-PAGE experiment.

These experiments show that cp7108 is a surface-exposed and immunoaccessible protein, and that it is a useful immunogen. These properties are not evident from the sequence alone.

### Example 42

The following C. pneumoniae protein (PID 4377287) was expressed <SEQ ID 83; cp7287>:

| | |
|---|---|
| 1 | MVAKKTVRSY RSSFSHSVIV AILSAGIAFE ASHSLHSSELD |
| | LGVFNKQFEE |
| 51 | HSAHVBEAQT SVLKGSDPVN PSQKESEKVL YTQVPLTQGS |
| | SGESLDLADA |
| 101 | NFLEHFQHLF EETTVFGIDQ KLVWSDLDTR NFSQPTQBPD |
| | TSNAVSEKIS |
| 151 | SDTKENRXDL ETEDPSRKSG LKEVSSDLPK SPETAVAAIS |
| | EDLEISENIS |
| 201 | ARDPLQGLAF FYKNTSSQSI SEKDSSFQGI IFSGSFANSG |
| | LGFENLKAPK |
| 251 | SGAAVYSDRD IVFENLVKGL SFISCESLED GSAAGVNIVV |
| | THCGDVTLTD |
| 301 | CATGLDLEAL IWVKDFSRGG AVFTAENHEV QNNLAGGILS |
| | VVGNXGAIVV |
| 351 | EKNSABKSNG GAFACGSFVY SNNENTALWK ENQALSGGAI |
| | SSASDIDIQG |
| 401 | NCSAIEFSGN QSLIALGEHI GLTDFVGGGA LAAQGTLTLR |
| | NNAVVQCVKN |
| 451 | TSKTHGGAIL AGTVDLNETI SEVAFKQNTA ALTGGALSAN |
| | DKVIIANNFG |
| 501 | EILFEQNEVR NHGGAIYCGC RSNPKLEQKD SGENINIIGN |
| | SGAITFLKNK |
| 551 | ASVLEVMTQA EDYAGGGALW GHNVLLDSNS GNIQFIGNIG |
| | GSTFWIGEYV |
| 601 | GGGAILSTDR VTISNNSGDV VFKGNRGQCL AQKYVAPQET |
| | APVESDASST |
| 651 | NKDEKSLNAC SHGDHYPPKT VEEEVPPSLL EEHPVVSSTD |
| | IRGGGAILAQ |
| 701 | HIFITDNTGN LRFSGNLGGG EESSTVGDLA IVGGGALLST |
| | NEVNVCSNQN |
| 751 | VVPSDNVTSN GCDSGGAILA KKVDISANHS VEFVSNGSGK |
| | FGGAVCALNE |
| 801 | SVNITDNGSA VSFSKNRTRL GGAGVAAPQG SVTICGNQGN |
| | IAFKENFVFG |
| 851 | SENQRSGGGA IIANSSVNIQ DNAGDILFVS NSTGSYGGAI |
| | FVGSLVASEG |
| 901 | SNPRTLTITG NSGDILFAKN STQTAASLSE KDSFGGGAIY |
| | TQNLKIVKNA |
| 951 | GNVSFYGNRA PSGAGVQIAD GGTVCLEAFG GDILFEGNIN |
| | FDGSFNAIHL |
| 1001 | CGNDSKIVEL SAVQDKNIIF QDAITYEENT IRGLPKDKVS |
| | PLSAPLSIFN |
| 1051 | SKPQDDSAQH HEGTIRFSRG VSKIPQIAAI QEGPLALSQN |
| | AELWLAGLKQ |
| 1101 | ETGSSIVLSA GSILRIFDSQ VDSSAPLPTE NKEETLVSAG |
| | VQINMSSPTP |
| 1151 | NKDKAVDTPV LADIISITVD LSSFVPEQDG TLPLPPEIII |
| | PKGTKLHSNA |
| 1201 | IDLKIIPPTN VGYENHALLS SHKDIPLISL KTAEGMTGTP |
| | TADASLSNIK |
| 1251 | IDVSLPSITP ATYGHTGVWS ESKMEDGRLV VGWQPTGYKL |
| | NPEKQGALVL |
| 1301 | NNLWSHYTDL RALKQEIFAH HTIAQRMELD FSTNVWGSGL |
| | GVVEDCQNIG |
| 1351 | EFDGFKHHLT GYALGLDTQL VEDFLIGGCF SQFFGKTESQ |
| | SYKAKNDVKS |
| 1401 | YMGAAYAGIL AGPWLIKGAF VYGNINNDLT TDYGTLGIST |
| | GSWIGKGFIA |
| 1451 | GTSIDYRYIV NPRRFISAIV STVVPFVBAE YVRIDLPEIS |
| | EQGKEVRTFQ |
| 1501 | KTRFENVAIP FGFALEHAYS RGSRABVNSV QLAYVFDVYR |
| | KGPVSLITLK |
| 1551 | DAAYSWKSYG VDIPCKAWKA RLSNNTEWNS YLSTYLAFNY |
| | EWREDLIAYD |
| 1601 | FNGGIRIIF* |

A predicted signal peptide is highlighted.

The cp7287 nucleotide sequence <SEQ ID 84> is:

| | |
|---|---|
| 1 | ATGGTAGCGA AAAAAACAGT ACGATCTTAT AGGTCTTCAT |
| | TTTCTCATTC |
| 51 | CGTAATAGTA GCAATATTGT CAGCAGGCAT TGCTTTTGAA |
| | GCACATTCCT |
| 101 | TACACAGCTC AGAACTAGAT TTAGGTGTAT TCAATAAACA |
| | GTTTGAGGAA |
| 151 | CATTCTGCTC ATGTTGAAGA GGCTCAAACA TCTGTTTTAA |
| | AGGGATCAGA |
| 201 | TCCTGTAAAT CCCTCTCAGA AAGAATCCGA GAAGGTTTTG |
| | TACACTCAAG |
| 251 | TGCCTCTTAC CCAAGGAAGC TCTGGAGAGA GTTTGGATCT |
| | CGCCGATGCT |
| 301 | AATTTCTTAG AGCATTTTCA GCATCTTTTT GAAGAGACTA |
| | CAGTATTTGG |
| 351 | TATCGATCAA AAGCTGGTTT GGTCAGATTT AGATACTAGG |
| | AATTTTTCCC |
| 401 | AACCCACTCA AGAACCTGAT ACAAGTAATG CTGTAAGTGA |
| | GAAAATCTCC |
| 451 | TCAGATACCA AAGAGAATAG AAAAGACCTA GAGACTGAAG |
| | ATCCTTCAAA |
| 501 | AAAAAGTGGC CTTAAAGAAG TTTCATCAGA TCTCCCTAAA |
| | AGTCCTGAAA |
| 551 | CTGCAGTAGC AGCTATTTCT GAAGATCTTG AAATCTCAGA |
| | AAACATTTCA |
| 601 | GCAAGAGATC CTCTTCAGGG TTTAGCATTT TTTTATAAAA |
| | ATACATCTTC |
| 651 | TCAGTCTATC TCTGAAAAGG ATTCTTCATT TCAAGGAATT |
| | ATCTTTTCTG |
| 701 | GTTCAGOAGC TAATTCAGGG CTAGGTTTTG AAAATCTTAA |
| | GGCGCCGAAA |
| 751 | TCTGGGGCTG CAGTTTATTC TGATCGAGAT ATTGTTTTTG |
| | AAAATCTTGT |
| 801 | TAAAGGATTG AGTTTTATAT CTTGTGAATC TTTAGAAGAT |
| | GGCTCTGCCG |
| 851 | CAGGTGTAAA CATTGTTGTG ACCCATTGTG GTGATGTAAC |
| | TCTCACTGAT |
| 901 | TGTGCCACTG GTTTAGACCT TGAAGCTTTA CGTCTGGTTA |
| | AAGATTTTTC |
| 951 | TCGTGGAGGA GCTGTTTTCA CTGCTCGCAA CCATGAAGTG |
| | CAAAATAACC |
| 1001 | TTGCAGGTGG AATTCTATCC GTTGTAGGCA ATAAAGGAGC |
| | TATTGTTGTA |
| 1051 | GAGAAAAATA GTGCTGAGAA GTCCAATGGA GGAGCTTTTG |
| | CTTGCGGAAG |
| 1101 | TTTTGTTTAC AGTAACAACG AAAACACCGC CTTGTGGAAA |
| | GAAAATCAAG |
| 1151 | CATTATCAGG AGGAGCCATA TCCTCAGCAA GTGATATTGA |
| | TATTCAAGGG |
| 1201 | AACTGTAGCG CTATTGAATT TTCAGGAAAC CAGTCTCTAA |
| | TTGCTCTTGG |
| 1251 | AGAGCATATA GGGCTTACAG ATTTTGTAGG TGGAGGAGCT |
| | TTAGCTGCTC |
| 1301 | AAGGGACGCT TACCTTAAGA AATAATGCAG TAGTGCAATG |
| | TGTTAAAAAC |
| 1351 | ACTTCTAAAA CACATGGTGG AGCTATTTTA GCAGGTACTG |
| | TTGATCTCAA |
| 1401 | CGAAACAATT AGCGAAGTTG CCTTTAAGCA GAATACAGCA |
| | GCTCTAACTG |
| 1451 | GAGGTGCTTT AAGTGCAAAT GATAAGGTTA TAATTGCAAA |
| | TAACTTTGGA |
| 1501 | GAAATTCTTT TTGAGCAAAA CGAAGTGAGG AATCACGGAG |
| | GAGCCATTTA |
| 1551 | TTGTGGATGT CGATCTAATC CTAAGTTAGA ACAAAAGGAT |
| | TCTGGAGAGA |
| 1601 | ACATCAATAT TATTGGAAAC TCCGGAGCTA TCACTTTTTT |
| | AAAAAATAAG |
| 1651 | GCTTCTGTTT TAGAAGTGAT GACACAAGCT GAAGATTATG |
| | CTGGTGGAGG |
| 1701 | CGCTTTATGG GGGCATAATG TTCTTCTAGA TTCCAATAGT |
| | GGGAATATTC |
| 1751 | AATTTATAGG AAATATAGGT GGAAGTACCT TCTGGATAGG |
| | AGAATATGTC |
| 1801 | GGTGGTGGTG CGATTCTCTC TACTGATAGA GTGACAATTT |
| | CTAATAACTC |
| 1851 | TGGAGATGTT GTTTTTAAAG GAAACAAAGG CCAATGTCTT |
| | GCTCAAAAAT |
| 1901 | ATGTAGCTCC TCAAGAAACA GCTCCCGTGG AATCAGATGC |
| | TTCATCTACA |
| 1951 | AATAAAGACG AGAAGAGCCT TAATGCTTGT AGTCATGGAG |
| | ATCATTATCC |
| 2001 | TCCTAAAACT GTAGAAGAGG AAGTGCCACC TTCATTGTTA |
| | GAAGAACATC |
| 2051 | CTGTTGTTTC TTCGACAGAT ATTCGTGGTG GTGGGGCCAT |
| | TCTAGCTCAA |
| 2101 | CATATCTTTA TTACAGATAA TACAGGAAAT CTGAGATTCT |
| | CTGGGAACCT |
| 2151 | TGGTGGTGGT GAAGAGTCTT CTACTGTCGG TGATTTAGCT |
| | ATCGTAGGAG |
| 2201 | GAGGTGCTTT GCTTTCTACT AATGAAGTTA ATGTTTGCAG |
| | TAACCAAAAT |
| 2251 | GTTGTTTTTT CTGATAACGT GACTTCAAAT GGTTGTGATT |
| | CAGGGGGAGC |
| 2301 | TATTTTAGCT AAAAAAGTAG ATATCTCCGC GAACCACTCG |
| | GTTGAATTTG |
| 2351 | TCTCTAATGG TTCAGGGAAA TTCGGTGGTG CCGTTTGCGC |
| | TTTAAACGAA |
| 2401 | TCAGTAAAAA TTACGGACAA TGGCTCGGCA GTATCATTCT |
| | CTAAAAATAG |
| 2451 | AACACGTCTT GGCGGTGCTG GAGTTGCAGC TCCTCAAGGC |
| | TCTGTAACGA |
| 2501 | TTTGTGGAAA TCAGGGAAAC ATAGCATTTA AAGAGAACTT |
| | TGTTTTTGCC |
| 2551 | TCTGAAAATC AAAGATCAGG TGGAGGAGCT ATCATTGCTA |
| | ACTCTTCTGT |
| 2601 | AAATATTCAG GATTACGCAG GAGATATOCT ATTTGPAAGT |
| | AACTCTACGG |
| 2651 | GATCTTATGG AGGTGCTATT TTTGTAGGAT CTTTGGTTGC |
| | TTCTGAAGGC |
| 2701 | AGCAACCCAC GAACGCTTAC AATTACAGGC AACAGTGGGG |
| | ATATCCTATT |
| 2751 | TGCTAAAAAT AGCACGCAAA CAGCCGCTTC TTTATCAGAA |
| | AAAGATTCCT |
| 2801 | TTGGTGGAGG GGCCATCTAT ACACAAAACC TCAAAATTGT |
| | AAAGAATGCA |
| 2851 | GGGAACGTTT CTTTCTATGG CAACAGAGCT CCTAGTGGTG |
| | CTGGTGTCCA |
| 2901 | AATTGCAGAC GGAGGAACTG TTTGTTTAGA GGCTTTTGGA |
| | GGAGATATCT |
| 2951 | TATTTGAAGG GAATATCAAT TTTGATGGGA GTTTCAATGC |
| | GATTCACTTA |
| 3001 | TGCGGGAATG ACTCAAAAAT CGTAGAGCTT TCTGCTGTTC |
| | AAGATAAAAA |
| 3051 | TATTATTTTC CAAGATGCAA TTACTTATGA AGAGAACACA |
| | ATTCGTGCCT |
| 3101 | TGCCAGATAA AGATGTCAGT CCTTTAAGTG CCCCTTCATT |
| | AATTTTTAAC |
| 3151 | TCCAAGCCAC AAGATGACAG CGCTCAACAT CATGAAGGGA |
| | CGATACGGTT |
| 3201 | TTCTCGAGGG GTATCTAAAA TTCCTCAGAT TGCTGCTATA |
| | CAAGAGGGAA |
| 3251 | CCTTAGCTTT ATCACAAAAC GCAGAGCTTT GGTTGGCAGG |
| | ACTTAAACAG |
| 3301 | GAAACAGGAA GTTCTATCGT ATTGTCTGCG GGATCTATTC |
| | TCCGTATTTT |
| 3351 | TGATTCCCAG GTTGATAGCA GTGCGCCTCT TCCTACAGAA |
| | AATAAAGAGG |
| 3401 | AGACTCTTGT TTCTGCCGGA GTTCAAATTA ACATGAGCTC |
| | TCCTACACCC |
| 3451 | AATAAAGATA AAGCTGTAGA TACTCCAGTA CTTGCAGATA |
| | TCATAAGTAT |
| 3501 | TACTGTAGAT TTGTCTTCAT TTGTTCCTGA GCAAGACGGA |
| | ACTCTTCCTC |
| 3551 | TTCCTCCTGA AATTATCATT CCTAAGGGAA CAAAATTACA |
| | TTCTAATGCC |
| 3601 | ATAGATCTTA AGATTATAGA TCCTACCAAT GTGGGATATG |
| | AAAATCATGC |
| 3651 | TCTTCTAAGT TCTCATAAAG ATATTCCATT AATTTCTCTT |
| | AAGACAGCGG |
| 3701 | AAGGAATGAC AGGGACGCCT ACAGCAGATG CTTCTCTATC |
| | TAATATAAAA |
| 3751 | ATAGATGTAT CTTTACCTTC GATCACACCA GCAACGTATG |
| | GTCACACAGG |
| 3801 | AGTTTGGTCT GAAAGTAAAA TGGAAGATGG AAGACTTGTA |
| | GTCGGTTGGC |
| 3851 | AACCTACGGG ATATAAGTTA AATCCTGAGA AGCAAGGGGC |
| | TCTAGTTTTG |
| 3901 | AATAATCTCT GGAGTCATTA TACAGATCTT AGAGCTCTTA |
| | AGCAGGAGAT |
| 3951 | CTTTGCTCAT CATACGATAG CTCAAAGAAT GGAGTTAGAT |
| | TTCTCGACAA |
| 4001 | ATGTCTGGGG ATCAGGATTA GGTGTTGTTG AAGATTGTCA |
| | GAACATCGGA |
| 4051 | GAGTTTGATG GGTTCAAACA TCATCTCACA GGGTATGCCC |
| | TAGGCTTGGA |
| 4101 | TACACAACTA GTTGAAGACT TCTTAATTGG AGGATGTTTC |
| | TCACAGTTCT |
| 4151 | TTGGTAAAAC TGAAAGCCAA TCCTACAAAG CTAAGAACGA |
| | TGTGAAGAGT |
| 4201 | TATATGGGAG CTGCTTATGC GGGGATTTTA GCAGGTCCTT |
| | GGTTAATAAA |
| 4251 | AGGAGCTTTT GTTTACGGTA ATATAAACAA CGATTTGACT |
| | ACAGATTACG |
| 4301 | GTACTTTAGG TATTTCAACA GGTTCATGGA TAGGAAAAGG |
| | GTTTATCGCA |
| 4351 | GGCACAAGCA TTGATTACCG CTATATTGTA AATCCTCGAC |
| | GGTTTATATC |
| 4401 | GGCAATCGTA TCCACAGTGG TTCCTTTTGT AGAAGCCGAG |
| | TATGTCCGTA |
| 4451 | TAGATCTTCC AGAAATTAGC GAACAGGGTA AAGAGGTTAG |
| | AACGTTCCAA |
| 4501 | AAAACTCGTT TTGAGAATGT CGCCATTCCT TTTGGATTTG |
| | CTTTAGAACA |
| 4551 | TGCTTATTCG CGTGGCTCAC GTGCTGAAGT GAACAGTGTA |
| | CAGCTTGCTT |
| 4601 | ACGTCTTTGA TGTATATCGT AAGGGACCTG TCTCTTTGAT |
| | TACACTCAAG |
| 4651 | GATGCTGCTT ATTCTTGGAA GAGTTATGGG GTAGATATTC |
| | CTTGTAAAGC |
| 4701 | TTGGAAGGCT CGCTTGAGCA ATAATACGGA ATGGAATTCA |
| | TATTTAAGTA |
| 4751 | CGTATTTAGC GTTTAATTAT GAATGGAGAG AAGATCTGAT |
| | AGCTTATGAC |
| 4801 | TTCAATGGTG GTATCCGTAT TATTTTCTAG |

The PSORT algorithm predicts an inner membrane location (0.106).

The protein was expressed in E. coli and purified as a GST-fusion product, as shown in FIG. 42A. The recombinant protein was used to immunise mice, whose sera were used in a Western blot (FIG. 42B) and for FACS analysis (FIG. 42C). A his-tagged protein was also expressed.

The cp7287 protein was also identified in the 2D-PAGE experiment and showed good cross-reactivity with human sera, including sera from patients with pneumonitis.

These experiments show that cp7287 is a surface-exposed and immunoaccessible protein, and that it is a useful immunogen. These properties are not evident from the sequence alone.

### Example 43

The following C. pneumoniae protein (PID 4377105) was expressed <SEQ ID 85; cp7105>:

| | |
|---|---|
| 1 | MSLYQKWWNS QLKKSLCYST VAALIFMIPS QESFADSLID |
| | LNLGLDPSVE |
| 51 | CLSGDGAFSV GYFTKAGSTP VEYQPFKYDV SKKTFTILSV |
| | ETANQSGYAY |
| 101 | GISYDGTITV GTCLSGAGKY NGAKWSADGT LTPLTGITGG |
| | TSHTEARAIS |
| 151 | KDTQVIEGFS YDASGQPKAV QWASGATTVT QLADISGGSR |
| | SSYAYAISDD |
| 201 | GTIIVGSMES TITRKTTAVK WVNNVPTYLG TLGGDASTGL |
| | YISGDGTVIV |
| 251 | GAANTATVTN GNQESHAYMY KDNQMKD* |

The cp7105 nucleotide sequence <SEQ ID 86> is:

| | |
|---|---|
| 1 | GTGAGTCTAT ATCAAAAATG GTGGAACAGT CAGTTAAAGA |
| | AGAGCCTCTG |
| 51 | CTATTCGACT GTTGCTGCTC TAATATTTAT GATTCCTTCT |
| | CAAGAATCCT |
| 101 | TTGCAGATAG TCTTATAGAT TTAAATTTAG GTTTAGATCC |
| | TTCGGTCGAA |
| 151 | TGTCTGTCAG GAGATGGTGC ATTTTCTGTT GGGTATTTTA |
| | CTAAGGCGGG |
| 201 | ATCGACTCCC GTAGAATATC AGCCGTTTAA ATACGACGTA |
| | TCTAAGAAGA |
| 251 | CATTCACAAT CCTTTCCGTA GAAACGGCAA ATCAGAGCGG |
| | CTATGCTTAC |
| 301 | GGAATCTCCT ACGATGGCAC GATCACTGTA GGAACGTGTA |
| | GCCTAGGTGC |
| 351 | AGGAAAATAT AACGGCGCAA AATGGAGTGC GGATGGCACT |
| | TTAACACCCT |
| 401 | TAACTGGAAT CACGGGGGGG ACGTCACATA CGGAAGCGCG |
| | TGCGATTTCT |
| 451 | AAGGATATTC AGGTGATCGA GGGTTTCTCA TATGATGCTT |
| | CAGGGCAACC |
| 501 | CAAGGCTGTG CAGTAGCGAC GCGGAGCGAC TACAGTAACA |
| | CAATTAGCAG |
| 551 | ATATTTCAGG AGGCTCTAGA AGCTCTTATG CGTTTGCTAT |
| | ATCTGATGAT |
| 601 | GGCACGATTA TTGTTGGGTC TATGGAGAGC ACGATAACAA |
| | GGAAAACTAC |
| 651 | AGCTGTAAAA TGGGTAAATA ATGTTCCTAC GTATCTGGGA |
| | ACCTTAGGAG |
| 701 | GAGATGCTTC TACAGGTCTT TATATTTCTG GAGACGGCAC |
| | CGTGATTGTA |
| 751 | GGTGCGGCAA ATACAGCAAC TGTAACCAAT GGGAATCAGG |
| | AATCCCACGC |
| 801 | CTATATGTAT AAAGATAACC AAATGAAAGA TTGA |

The PSORT algorithm predicts an inner membrane location (0.100).

The protein was expressed in E. coli and purified as a GST-fusion product, as shown in FIG. 43A. The recombinant protein was used to immunise mice, whose sera were used in a Western blot (FIG. 43B) and for FACS analysis (FIG. 43C). A his-tagged protein was also expressed.

This protein also showed good cross-reactivity with human sera, including sera from patients with pneumonitis.

These experiments show that cp7105 is a surface-exposed and immunoaccessible protein, and that it is a useful immunogen. These properties are not evident from the sequence alone.

### Example 44

The following C. pneumoniae protein (PID 4376802) was expressed <SEQ ID 87; cp6802>:

| | |
|---|---|
| 1 | MSNQLQPCIS LGCVSYINSF PLSLQLIKRN DIRCLAPPA |
| | DLLNLLIEGK |
| 51 | LDVALTSSLG AISHNLGYVP GFGIAANQRI LSVNLYAAPT |
| | FFNSPQPRIA |
| 101 | ATLESRSSIG LLKVLCRHLW RIPTPHILRF ITTKVLRQTP |
| | ENYDGLLLIG |
| 151 | DAALQHPVLP GFVTYDLASG WYDLTKLPFV FALLLHSTSW |
| | KEHPLPNLAM |
| 201 | EEALQQFESS PEEVLKEAHQ HTGLPPSLLQ EYYALCQYRL |
| | GEEHYESFEK |
| 251 | FREYYGTLYQ QARL* |

A predicted signal peptide is highlighted.

The cp6802 nucleotide sequence <SEQ ID 88> is:

| | |
|---|---|
| 1 | ATGTCTAACC AACTCCAGCC ATGTATAAGC TTAGGCTGCG |
| | TAAGTTATAT |
| 51 | TAATTCCTTT CCGCTGTCCC TACAACTCAT AAAAAGAAAC |
| | GATATTCGCT |
| 101 | GTGTTCTTGC TCCCCCTGCA GACCTCCTCA ACTTGCTAAT |
| | CGAAGGGAAA |
| 151 | CTCGATGTTG CTTTGACCTC ATCCCTAGGA GCTATCTCTC |
| | ATAACTTGGG |
| 201 | GTATGTCCCC GGCTTTGGAA TTGCAGCAAA CCAACGTATC |
| | CTCAGTGTAA |
| 251 | ACCTCTATGC AGCTCCCACT TTCTTTAACT CACCGCAACC |
| | TCGGATTGCC |
| 301 | GCAACTTTAG AAAGTCGCTC CTCThTAGGA CTCTTAAAAG |
| | TGCTTTGTCG |
| 351 | TCATCTCTGG CGCATCCCAA CTCCTCATAT CCTAAGATTC |
| | ATAACTACAA |
| 401 | AAGTACTCAG ACAAACCCCT GAAAATTATG ATGGCCTCCT |
| | CCTAATCGGA |
| 451 | GATCCAGCGC TACAACATCC TGTACTTCCT GGATTTGTAA |
| | CCTATGACCT |
| 501 | TGCCTCGGGG TGGTATGATC TTACAAAGCT ACCTTTTGTA |
| | TTTGCTCTTC |
| 551 | TTCTACACAG CACCTCTTGG AAAGAACATC CCCTACCCAA |
| | CCTTGCGATG |
| 601 | GAAGAAGCCC TCCAACAGTT CGAATCTTCA CCCGAAGAAG |
| | TCCTTAAAGA |
| 651 | AGCTCATCAA CATACAGGTC TGCCCCCTTC TCTTCTTCAA |
| | GAATACTATG |
| 701 | CCCTATGCCA GTACCGTCTA GGAGAAGAAC ACTACGAAAG |
| | CTTTGAAAAA |
| 751 | TTCCGGGAAT ATTATGGAAC CCTCTACCAA CAAGCCCGAC |
| | TGTAA |

The PSORT algorithm predicts an inner membrane location (0.060).

The protein was expressed in E. coli and purified as a GST-fusion product, as shown in FIG. 44A. The recombinant protein was used to immunise mice, whose sera were used in a Western blot (FIG. 44B) and for FACS analysis (FIG. 44C). A his-tagged protein was also expressed.

These experiments show that cp6802 is a surface-exposed and immunoaccessible protein, and that it is a useful immunogen. These properties are not evident from the sequence alone.

### Example 45

The following C. pneumoniae protein (PID 4376390) was expressed <SEQ ID 89; cp6390>:

| | |
|---|---|
| 1 | MVFSYYCMGL FFFSQAISSC GLLVSLQVGL GLSVLGVLLL |
| | LLAGLLLFKI |
| 51 | QSMLREVPKA PDLLDLEDAS EELRVKASRS LASLPKEISQ |
| | LESYIRSAAN |
| 101 | DLNTIKTWPH KDQRLVETVS RKLERLAAAQ NYMISELCEI |
| | SEILEEEEHH |
| 151 | LILAQESLEW IGKSLFSTFL DMESFLNLSH LSEVRPYLAV |
| | NDPRLLEITE |
| 201 | ESWEVVSHFI NVTSAFKKAQ ILFKNNEHSR MKKKLESVQE |
| | LLETFIYKSL |
| 251 | KRSYRELGCL SEKMRIIHDN PLFPWVQDQQ KYAHAKNEFG |
| | EIARCLEEFE |
| 301 | KTEFWLDEBC AISYMDCWDF LNESIQNKKS RVDRDYISTK |
| | KIALKDRART |
| 351 | YAKVLLEENP TTEGKIDLQD AQRAFERQSQ EFYTLEHTET |
| | KVRLEALQQC |
| 401 | FSDLREATNV RQVRFTNSEN ANDLKESFEK IDKERVRYQK |
| | EQRLYWETID |
| 451 | RNEQELREEI GESLRLQNRR KGYRAGYDAG RLKGLLRQWK |
| | KNLRDVEAHL |
| 501 | EDATMDFEHE VSKSELCSVR ARLEVLEEEL MDMSPKVADI |
| | EELLSYEERC |
| 551 | ILPIRENLER AYLQYNKCSE ILSKAKFFFP EDEQLLVSEA |
| | NLREVGAQLE |
| 601 | QVQGKCQERA QKFAIFEKHI QEQKSLIKEQ VRSFDLAGVG |
| | FLKSELLSIA |
| 651 | CNLYIKAVVK ESIPVDVPCM QLYYSYYEDN EAVVRNRLLN |
| | MTERYQNFKR |
| 701 | SLUSIQFNGD VLLRDPVYQP EGHETRLKER ELQETTLSCK |
| | KLKVAQDRLS |
| 751 | ELESRLSRR |

A predicted signal peptide is highlighted.

The cp6390 nucleotide sequence <SEQ ID 90> is:

| | |
|---|---|
| 1 | TTGGTATTCT CATACTATTG CATGGGATTA TTTTTTTTCT |
| | CTGGAGCTAT |
| 51 | TTCTAGTTGT GGTCTTTTAG TGTCTCTAGG AGTTGGTTTA |
| | GGACTTAGTG |
| 101 | TTTTAGGAGT ACTTTTACTT CTCTTAGCAG GTCTTTTGCT |
| | TTTTAAGATC |
| 151 | CAAAGTATGC TTCGAGAGGT GCCTAAGGCT CCTGATCTAT |
| | TAGATTTAGA |
| 201 | AGAPGCAAGT GAACGGCTTA GAGTAAAGGC TAGCCGTTCT |
| | TTACCAAGCC |
| 251 | TCCCGAAGGA AATCAGTCAG CTAGAGAGCT ACATTCGTTC |
| | TGCAGCTAAT |
| 301 | GATCTAAATA CAATTAAGAC TTGGCCGCAT AAAGATCAAA |
| | GACTCGTCGA |
| 351 | GACCGTGTCA CQAAAATTAG AGCGTCTGGC AGCTGCTCAA |
| | AACTATATGA |
| 401 | TTTCTGAACT CTGCGAGATT AGTGAGATTC TTGAGGAAGA |
| | GGAGCATCAT |
| 451 | CTAATTTTGG CTCAGGAATC TCTAGAATGG ATAGGTAAGA |
| | GTCTATTTTC |
| 501 | TACCTTTCTG GACATGGAAT CTTTTTTAAA TTTGAGCCAT |
| | CTATCTGAAG |
| 551 | TGCGTCCGTA CTTAGCTGTA AATGATCCTA GATTATTAGA |
| | AATTACCGAA |
| 601 | GAATCTTGGG AAGTAGTGAG TCATTTCATA AATGTAACGT |
| | CTGCTTTTAA |
| 651 | GAAAGCTCAG ATTCTTTTTA AGAACAACGA ACATTCTCGG |
| | ATGAAGAAGA |
| 701 | AGTTAGAAAG TGTTCAAGAG TTACTGGAAA CATTTATTTA |
| | TAAGAGTTTA |
| 751 | AAGAGAAGTT ATCGAGAATT AGGATGCTTA AGTGAAAAGA |
| | TGAGAATCAT |
| 801 | TCACGACAAT CCTCTCTTCC CTTGGGTGCA AGATCAGCAG |
| | AAGTATGGTC |
| 851 | ATGCTAAGAA TGAATTTGGA GAGATTGGGA GGTGTTTAGA |
| | GGAGTTTGAA |
| 901 | AAGACGTTCT TCTGGTTGGA TGAGGAGTGT GCTATTTCTT |
| | ACATGGACTG |
| 951 | TTGGGATTTT CTAAATGAGT CTATTCAGAA TAAGAAGTCC |
| | AGAGTAGATC |
| 1001 | GAGATTATAT ATCCACGAAG AAAATTGCAT TAAAGGATAG |
| | AGCCCGCACT |
| 1051 | TATGCTAAGG TTCTTTTAGA AGAGAATCCG ACTACAGAGG |
| | GTAAAATAGA |
| 1101 | TTTGCAAGAC GCTCAAAGAG CCTTTGAGOG TCAAAGTCAG |
| | GAGTTTTATA |
| 1151 | CACTAGAGCA TACGGAAACA AAGGTGAGAC TAGAAGCACT |
| | TCAACAGTGC |
| 1201 | TTCTCGGATC TTAGGGAGGC GACGAACGTA AGGCAAGTTA |
| | GGTTTACAAA |
| 1251 | TTCTGAAAAT GCGAATGATT TAAAGGAGAG TTTCGAGAAG |
| | ATAGATAAAG |
| 1301 | AGCGTGTGCG ATATCAAAAA GAGCAAAGGC TCTATTGGGA |
| | AACAATAGAT |
| 1351 | CGCAATGAGC AAGAGCTTAG GGAAGAGATT GGGGAGTCGC |
| | TTCGTTTACA |
| 1401 | AAATCGGAGA AAAGGGTATA GGGCTGGATA TGATGCTGGG |
| | CGTTTAAAAG |
| 1451 | GTTTGTTGCG TCAGTGGAAG AAAAAACTCC GCGATGTGGA |
| | AGCCCACCTT |
| 1501 | GAAGATGCAA CTATGGATTT TGAGCATGAA GTAAGCAAGA |
| | GCGAATTGTG |
| 1551 | CAGTGTTCGG GCGAGGCTCG AGGTTCTAGA AGAAGAGCTG |
| | ATGGATATGT |
| 1601 | CTCCTAAAGT TGCGGATATA GAAGAGTTGT TGTCCTATGA |
| | AGAGCGTTGT |
| 1651 | ATTCTTCCTA TTAGGGAAAA TTTAGAAAGG GCATACCTCC |
| | AATATAATAA |
| 1701 | GTGTTCTGAA ATTTTATCCA AGGCAAAGTT CTTCTTTCCG |
| | GAAGACGAGC |
| 1751 | AATTGCTAGT TTCGGAAGCG AATCTAAGAG AGGTGGGTGC |
| | CCAGTTAAAA |
| 1801 | CAAGTACAGG GAAAATGTCA AGAGAGGGCC CAAAAGTTCG |
| | CAATATTTGA |
| 1851 | AAAGCATATT CAGGAGCAGA AAAGCCTTAT TAAAGAGCAA |
| | GTGCGGAGTT |
| 1901 | TTGATCTAGC GGGAGTTGGG TTTTTAAAGA GTGAGCTTCT |
| | TAGTATTGCT |
| 1951 | TGTAACCTTT ATATAAAGGC GGTTGTTAAG GAGTCTATAC |
| | CAGTTGATGT |
| 2001 | GCCTTGTATG CAGTTATATT ATAGTTATTA CGAAGATAAT |
| | GAAGCTGTAG |
| 2051 | TGCGAAACCG CCTTTTAAAT ATGACGGAGA GGTATCAAAA |
| | TTTTAAAAGG |
| 2101 | AGTTTGAATT CCATACAATT TAATGGTGAC GTTCTTTTAC |
| | GGGATCCGGT |
| 2151 | CTATCAACCT GAAGGTCATG AGACCAGGCT AAAGGAACGG |
| | GAGCTACAAG |
| 2201 | AAACAACTTT GTCTTGTAAG AAATTAAAAG TGGCTCAAGA |
| | TCGTCTTTCT |
| 2251 | GAATTAGAGT CAAGGCTGTC TAGGAGATAG |

The PSORT algorithm predicts a periplasmic location (0.932).

The protein was expressed in E. coli and purified as a GST-fusion product, as shown in FIG. 45A. The recombinant protein was used to immunise mice, whose sera were used in a Western blot (FIG. 45B) and for FACS analysis (FIG. 45C). A his-tagged protein was also expressed.

This protein also showed good cross-reactivity with human sera, including sera from patients with pneumonitis.

These experiments show that cp6390 is a surface-exposed and immunoaccessible protein, and that it is a useful immunogen. These properties are not evident from the sequence alone.

### Example 46

The following C. pneumoniae protein (PID 4376272) was expressed <SEQ ID 91; cp6272>:

| | |
|---|---|
| 1 | MKRCFLFLAS FVLMGSSADA LTHQEAVKKK NSYLSHFKSV |
| | SGIVTIEDGV |
| 51 | LNIHNNLRIQ ANKVYVENTV GQSLKLVAHG NVMVNYTAKT |
| | LVCDYLEYYE |
| 101 | DTDSCLLTNG RPMIYPWFLG GSMITLTPET IVIRRGYIST |
| | SEGPKKDLCL |
| 151 | SGDYLEYSSD SLLSIGKTTL RVCRIPILFL PPFSIMPMEI |
| | PKPPINFRGG |
| 201 | TGGFLGSYLG MSYSPISRKR FSSTFFLDSF FKHGVGMGFN |
| | LHCSQKQVPE |
| 251 | NVFNMKSYYA HRLAIDMAEA HDRYRLHGDF CFTHKHVNFS |
| | GEYHLSDSWE |
| 301 | TVADIFPNNF MLKNTGPTRV DCTWNDNYFE GYLTSSVKVN |
| | SFQNANQELP |
| 351 | YLTLRQYPIS IYNTGVYLBN IVECGYLNFA FSDHIVGBNF |
| | SSLRLAARPK |
| 401 | LHKTVPLPIG TLSSTLGSSL IYYSDVPEIS SRHSQLSAKL |
| | QLDYRFLLHK |
| 451 | SYIQRRHIIE PFVTFITETR PLAKNEDHYI FSIQDAFHSL |
| | NLLKAGIDTS |
| 501 | VLSKTNPRFP RIHAKLWTTH ILSNTESKPT FPKTACELSL |
| | PFGKKNTVSL |
| 551 | DAEWIWKKHC WDHMNIRWEW IGNDNVAMTL ESLHRSKYSL |
| | IKCDRENFIL |
| 601 | DVSRPIDQLL DSPLSDHPNL ILGKLFVRPH PCWNYRLSLR |
| | YGWHRQDTPN |
| 651 | YLEYQMILGT KIFEHWQLYG VYERREADSR FFFFLKLDKP |
| | KKPPF* |

A predicted signal peptide is highlighted.

The cp6272 nucleotide sequence <SEQ ID 92> is:

| | |
|---|---|
| 1 | ATGAAACGTT GCTTCTTATT TCTAGCTTCC TTTGTTCTTA |
| | TGGGTTCCTC |
| 51 | AGCTGATGCT TTGACTCATC AAGAGGCTGT GAAAAAGAAA |
| | AACTCCTATC |
| 101 | TTAGTCACTT TAAGAGTGTT TCTGGGATTG TQACCATCGA |
| | AGATGGGGTA |
| 151 | TTGAATATCC ATAACAACCT GCGGATACAA GCCAATAAAG |
| | TGTATGTAGA |
| 201 | AAATACTGTG GGTCAAAGCC TGAAGCTTGT CGCACATGGC |
| | AATGTTATGG |
| 251 | TGAACTATAG GGCAAAAACC CTAGTTTGTG ATTACOTAGA |
| | GTATTACGAA |
| 301 | GATACAGACT CTTGTCTTCT TACTAATGGA AGATTCGCGA |
| | TGTATCCTTG |
| 351 | GTTTCTAGGG GGGTCTATGA TCACTCTAAC CCCAGAAACC |
| | ATAGTCATTC |
| 401 | GGAAGGGATA TATCTCTACC TCCGAGGGTC CCAAAAAAGA |
| | CCTGTGCCTC |
| 451 | TCCGGAGATT ACCTGGAATA TTCTTCAGAT AGTCTTCTTT |
| | CTATAGGGAA |
| 501 | GACAACATTA AGGGTGTGTC GCATTCCGAT ACTTTTCTTA |
| | CCTCCATTTT |
| 551 | CTATCATGCC TATGGAGATC CCTAAGCCTC CGATAAACTT |
| | TCGAGGAGGA |
| 601 | ACAGGAGGAT TTCTGGGATC CTATTTGGGG ATGAGCTACT |
| | CGCCGATTTC |
| 651 | TAGGAAGCAT TTCTCCTCGA CATTTTTCTT GGATAGCTTT |
| | TTCAAGCATG |
| 701 | GCGTCGGCAT GGGATTCAAC CTCCATTGTT CTCAGAAGCA |
| | GGTTCCTGAG |
| 751 | AATGTCTTCA ATATGAAAAG CTATTATGCC CACCGCCTTG |
| | CTATCGATAT |
| 801 | GGCAGAAGCT CATGATCGCT ATCGCCTACA CGGAGATTTC |
| | TGCTPCACGC |
| 851 | ATAAGCATGT AAATTTTTCT GGAGAATACC ATCTCAGCGA |
| | TAGTTGGGAA |
| 901 | ACTGTTGCTG ACATTTTCCC CAACAACTTC ATGTTGAAAA |
| | ATACAGGCCC |
| 951 | CACACGTGTC GATTGCACTT GGAATGACAA CTATTTTGAA |
| | GGGTATCTCA |
| 1001 | CCTCTTCTGT TAAGGTAAAC TCTTTCCAAA ATGCCAACCA |
| | AGAGCTCCCT |
| 1051 | TATTTAAQAT TAAGGCAGTA CCCGATTTCT ATTTATAATA |
| | CGGGAGTGTA |
| 1101 | CCTTGAAAAC ATCGTAGAAT GTGGGTATTT AAACTTPGCT |
| | TTTAGCGATC |
| 1151 | ATATCGTTGG CGAGAATTTC TCTTCACTAC GTCTTGCTGC |
| | GCGCCCTAAG |
| 1201 | CTCCATAAAA CTGTGCCTCT ACCTATAGGA ACGCTCTCCT |
| | CCACCCTAGG |
| 1251 | GAGTTCTCTG ATTTACTATA GCGATGTTCC TGAGATCTCC |
| | TCGCGCCATA |
| 1301 | GTCAGCTTTC CGCGAAGCTA CAACTTGATT ATCGCTTTCT |
| | ATTACATAAG |
| 1351 | TCCTACATTC AAAGACGCCA TATTATAGAG CCGTTCGTTA |
| | CCTTCATTAC |
| 1401 | AGAGACTCGT CCTCTAGCTA AGAATGAAGA TCATTATATC |
| | TTTTCTATTC |
| 1451 | AAGATGCCTT TCACTCCTTA AACCTTCTGA AAGCGGGTAT |
| | AGATACCTCG |
| 1501 | GTACTGAGTA AGACTAACCC TCGATTCCCG AGAATCCATG |
| | CGAAGCTGTG |
| 1551 | GACTACCCAC ATCTTGAGCA ATACAGAAAG CAAACCCACG |
| | TTTCCCAAAA |
| 1601 | CTGCATGCGA GCTATCTCTA CCTTTTGGAA AGAAAAATAC |
| | AGTCTCCTTA |
| 1651 | GATGCTGAAT GGATTTGGAA AAAGCACTGT TGGGATCACA |
| | TGAACATACG |
| 1701 | TTGGGAGTGG ATCGGAAATG ACAATGTGGC TATGACTCTA |
| | GAATCCCTGC |
| 1751 | ATAGAAGCAA ATACAGCCTG ATTAAGTGTG ACAGGGAGAA |
| | CTTCATTTTA |
| 1801 | GATGTCAGCC GTCCCATTGA CCAGCTTTTA GACTCCCCTC |
| | TCTCTGATCA |
| 1851 | TAGGAATCTC ATTTTAGGGA AATTATTTGT ACGACCTCAT |
| | CCCTGTTGGA |
| 1901 | ATTACCGCTT ATCCTTACGC TATGGCTGGC ATCGCCAGGA |
| | CACTCCGAAC |
| 1951 | TACCTAGAAT ACCAGATGAT TCTAGGGACG AAGATCTTCG |
| | AACATTGGCA |
| 2001 | GCTCTATGGG GTGTATGAAC GCCGAGAAGC AGATAGTCGA |
| | TTTTTCTTCT |
| 2051 | TCTTAAAGCT CGACAAACCT AAAAAACCTC CCTTCTAA |

The PSORT algorithm predicts an outer membrane location (0.48).

The protein was expressed in E. coli and purified as a GST-fusion product, as shown in FIG. 46A. The recombinant protein was used to immunise mice, whose sera were used in a Western blot and for FACS analysis (FIG. 46B). A his-tagged protein was also expressed.

This protein also showed good cross-reactivity with human sera, including sera from patients with pneumonitis.

These experiments show that cp6272 is a surface-exposed and immunoaccessible protein, and that it is a useful immunogen. These properties are not evident from the sequence alone.

### Example 47

The following C. pneumoniae protein (PID 4377111) was expressed <SEQ ID 93; cp7111>:

| | |
|---|---|
| 1 | MFEAVIADIQ AREILDSRGY PTLHVKVTTS TGSVGEARVP |
| | SGASTGKKEA |
| 51 | LEFRDTDSPR YQGKGVLQAV KNVKEILFPL VKGCSVYBQS |
| | LIDSLMMDSD |
| 101 | GSPNKWPLGA NAILGVSLAT AHAAAATLRR PLYRYLGGCF |
| | ACSLPCPMNN |
| 151 | LINGGMHADN GLEFQEFMIR PIGASSIKEA VNMGADVFHT |
| | LKKLLRERGL |
| 201 | STGVGDEGGF APNLASNEEA LELLLLAIEK AGFTPGKDIS |
| | LALDCAASSF |
| 251 | YNVKTGTYDG RHYBEQIAIL SNLCDRYPID SIEDGLAEED |
| | YDGWALLTEV |
| 301 | LGEKVQIVGD DLFVTNPELI LEGISNGLAN SVLIKPNQIG |
| | TLTETVYAIK |
| 351 | LAQMAGYTTI ISHRSGETTD TTIADLAVAF NAGQIKTGSL |
| | SRSERVAKYN |
| 401 | RLMEIEEELG SEAIFTDSNV FSYEDSEE* |

A predicted signal peptide is highlighted.

The cp7111 nucleotide sequence <SEQ ID 94> is:

| | |
|---|---|
| 1 | ATGTTTGAAG CTGTCATTGC CGATATCCAG GCTAGGGAAA |
| | TCTTGGATTC |
| 51 | TCGCGGGTAT CCCACTTTAC ATGTTAAAGT AACCACTAGC |
| | ACAGGTTCTG |
| 101 | TTGGAGAAGC TCGGGTTCCT TCAGGAGCAT CCACAGGGAA |
| | AAAAGAAGCC |
| 151 | TTAGAGTTTC GTGATACAGA TTCTCCTCGT TATCAAGGCA |
| | AAGGGGTTTT |
| 201 | GCAAGCTGTA AAAAACGTAA AAGAAATTCT TTTTCCCCTC |
| | GTCAAGGGAT |
| 251 | GTAQTGTTTA TGAGCAATCC TTAATTGATT CTCTGATGAT |
| | GGATTCTGAC |
| 301 | GGCTCTCCGA ACAAAGAAAC TCTAGGGGCC AATGCTATTT |
| | TAGGAGTCTC |
| 351 | TCTAGCTACA GCACATGCAG CAGCAGCAAC ACTACGCAGA |
| | CCTCTGTATC |
| 401 | GTTATTTAGG AGGGTGTTTT GCCTGCAGTC TTCCCTGTCC |
| | TATGATGAAT |
| 451 | CTGATCAATG GAGGCATGCA TGCCGATAAC GGCTTGGAGT |
| | TCCAAGAATT |
| 501 | TATGATCCGT CCTATTGGAG CCTCTTCCAT CAAAGAAGCT |
| | GTCAACATGG |
| 551 | GTGCTGACGT TTTTCATACT TTGAAAAAAT TACTCCATGA |
| | AAGAGGCTTA |
| 601 | TCTACTGGAG TGGGTGACGA AGGAGGCTTC GCCCCGAATC |
| | TTGCTTCTAA |
| 651 | TGAAGAAGCT CTAGAGCTCC TAPTGCTGGC TATTGAAAAA |
| | GCAGGCTTTA |
| 701 | CTCCAGGAAA AGATATATCG CTAGCCTTAG ACTGCGCAGC |
| | ATCCTCATTC |
| 751 | TATAACGTAA AAACAGGCAC GTATGATGOG AGGCACTATG |
| | AAGAGCAAAT |
| 801 | CGCAATCCTT TCTAATTTAT GTGATCGCTA TCCTATAGAC |
| | TCCATAGAAG |
| 851 | ATGGTCTTGC TGAAGAAGAC TATGACGGGT GGGCCTTGTT |
| | AACTGAAGTT |
| 901 | CTTGGAGAAA AAGTACAGAT TGTGGGTGAT GACCTATTTG |
| | TTACAAATCC |
| 951 | GGAATTAATA TTAGAGGGTA TTAGCAATGG ATTAGCGAAC |
| | TCTGTGTTGA |
| 1001 | TTAAACCAAA TCAGATAGGG ACGCTTACTG AAACAGTGTA |
| | TGCTATCAAG |
| 1051 | CTTGCGCAAA TGGCTGGCTA TACTACAATT ATTTCTCATC |
| | GCTCAGGAGA |
| 1101 | AACTACGGAC ACTACGATTG CAGATCTTGC TGTTGCCTTC |
| | AACGCCGGTC |
| 1151 | AAATCAAAAC AGGCTCTTTA TCACGTTCTG AGCGTGTTGC |
| | AAAATACAAT |
| 1201 | AGACTCATGG AAATTGAAGA AGAGCTTGGA TCCGAAGCAA |
| | TTTTCACAGA |
| 1251 | TTCTAATGTA TTTTCTTAC GAGGATTCT GAGGAATAG |

The PSORT algorithm predicts an inner membrane location (0.100).

The protein was expressed in E. coli and purified as a GST-fusion product, as shown in FIG. 47A. The recombinant protein was used to immunise mice, whose sera were used in a Western blot (FIG. 47B) and for FACS analysis (FIG. 47C). A his-tagged protein was also expressed.

The cp7111 protein was also identified in the 2D-PAGE experiment and showed good cross-reactivity with human sera, including sera from patients with pneumonitis.

These experiments show that cp7111 is a surface-exposed and immunoaccessible protein, and that it is a useful immunogen. These properties are not evident from the sequence alone.

### Example 48

The following C. pneumoniae protein (PID 4455886) was expressed <SEQ ID 95; cp0010>:

| | |
|---|---|
| 1 | MKSQFSWLVL SSTLACDFTSC STVFAATAEN IGPSDSFDGS |
| | TNTGTYTPKN |
| 51 | TTTGIDYTLT GDITLQNLGD SAALTKGCFS DTTESLSFAG |
| | KGYSLSFLNI |
| 101 | KSSAEGAALS VTTDKNLSLT GFSSLTFLAA PSSVITTPSG |
| | KGAVKCGGDL |
| 151 | TFDNNGTILF KQDYCEENGG AISTKNLSLK NSTGSISFEG |
| | NKSSATGKKG |
| 201 | GAICATGTVD ITNNTAPTLF SNNIAEAAGG AINSTGNCTI |
| | TGNTSLVFSE |
| 251 | NSVTATAGNG GALSGDADVT ISGNQSVTFS GNQAVANGGA |
| | IYAKKLTLAS |
| 301 | GGGGVSPFLT IIVQGTTAGN GGAISILAAG ECSLSAEAGD |
| | ITFNGNAIVA |
| 351 | TTPQTTKENS IDIGSTAKIT NLRAISGHSI FFYDPITANT |
| | AADSTDTLNL |
| 401 | NKADAGNSTD YSGSIVFSGE KLSEDEAXVA DNLTSTLKQP |
| | VTLPAGNINL |
| 451 | KRGVTLDTKG FTQTAGSSVI MDAGTTLKAS TEEVTLTOLS |
| | IPVDSLGEGK |
| 501 | KVVIAASAAS KNVALSGPEL LLDNQGNAYE NHDLGKTQDF |
| | SFVQLSALGT |
| 551 | ATTTDVPAVP TVATPTHYGY QGTWGMTWVD DTASTPKTKT |
| | ATLAWTNTGY |
| 601 | LPNPBRQGPL VPNSLWGSFS DIQAIQQVIE RSALTLCSDR |
| | GFWAAGVANF |
| 651 | LDKDKRGEKR KYRHKSGGYA IGGAAQTCSE NLISFAFCQL |
| | FGSDKDFLVA |
| 701 | KNHTDTYAGA FYIQHITECS GFIGCLLDKL PGSWSHKPLV |
| | LEGQLAYSHV |
| 751 | SNDLRTKYTA YPEVKGSWGN NAFNMMLGAS SHSYPEYLHC |
| | FDTYAPYIKL |
| 801 | NLTYIRQDSF SEKGTEGRSF DDSNLFNLSL PIGVKFEKFS |
| | DCNDFSYDLT |
| 851 | LSYVPDLIRN DPKCTTALVI SGASWETYAN NLARQALQVR |
| | AGSHYAFSPM |
| 901 | FEVLGQFVFE VRGSSRIYNV DLGGKFQF* |

A predicted signal peptide is highlighted.

The cp0010 nucleotide sequence <SEQ ID 96> is:

| | |
|---|---|
| 1 | ATGAAATCGC AATTTTCCTG GTTAGTGCTC TCTTCGACAT |
| | TGGCATGTTT |
| 51 | TACTAGTTGT TCCACTGTTT TTGCTGCAAC TGCTGAAAAT |
| | ATAGGCCCCT |
| 101 | CTGATAGCTT TGACGGAAGT ACTAACACAG GCACCTATAC |
| | TCCTAAAAAT |
| 151 | ACGACTACTG GAATAGACTA TACTCTGACA GGAGATATAA |
| | CTCTGCAAAA |
| 201 | CCTTCCGGAT TCGGCAGCTT TAACGAAGGG TTGTTTTTCT |
| | GACACTACGG |
| 251 | AATCTTTAAG CTTTGCCGGT AAGGGGTACT CACTTTCTTT |
| | TTTAAATATT |
| 301 | AAGTCTAGTG CTGAAGGCGC AGCACTTTCT GTTACAACTG |
| | ATAAAAATCT |
| 351 | GTCGCTAACA GGATTTTCGA GTCTTACTTT CTTAGCGGCC |
| | CCATCATCGG |
| 401 | TAATCACAAC CCCCTCAGGA AAAGGTGCAG TTAAATGTGG |
| | AGGGGATCTT |
| 451 | ACATTTGATA ACAATGGAAC TATTTTATTT AAACAAGATT |
| | ACTGTGAGGA |
| 501 | AAATGGCGGA GCCATTTCTA CCAAGAATCT TTCTTTGAAA |
| | AACAGCACGG |
| 551 | GATCGATTTC TTTTCAAGGG AATAAATCGA GCGCAACAGG |
| | GAAAAAAGGT |
| 601 | GGGGCTATTT GTGCTACTGG TACTGTAGAT ATTACAAATA |
| | ATACGGCTCC |
| 651 | TACCCTCTTC TCGAACAATA TTGCTGAAGC TGCAGGTGGA |
| | GCTATAAATA |
| 701 | GCACAGGAAA CTGTACAATT ACAGGGAATA CGTCTCTTGT |
| | ATTTTCTGAA |
| 751 | AATAGTGTGA CAGCGACCGC AGGAAATGGA GGAGCTCTTT |
| | CTGGAGATGC |
| 801 | CQATGTTACC ATATCTGGGA ATCAGAGTGT AACTTTCTCA |
| | GGAAACCAAG |
| 851 | CTGTAGCTAA TGGCGGAGCC ATTTATGCTA AGAAGCTTAC |
| | ACTGGCTTCC |
| 901 | GGGGGGGGGG GGGTATCTCC TTTTCTAACA ATAATAGTCC |
| | AAGGTACCAC |
| 951 | TGCAGGTAAT GGTGGAGCCA TTTCTATACT GGCAGCTGGA |
| | GAGTGTAGTC |
| 1001 | TTTCAGCAGA AGCAGGGGAC ATTACCTTCA ATGGGAATGC |
| | CATTGTTGCA |
| 1051 | ACTACACCAC AAACTACAAA AAGAAATTCT ATTGACATAG |
| | GATCTACTGC |
| 1101 | AAAGATCACG AATTTACGTG CAATATCTGG GCATAGCATC |
| | TTTTTCTACG |
| 1151 | ATCCGATTAC TGCTAATACG GCTGCGGATT CTACAGATAC |
| | TTTAAATCTC |
| 1201 | AATAAGGCTG ATGCAGGTAA TAGTACAGAT TATAGTGGGT |
| | CGATTGTTTT |
| 1251 | TTCTGGTGAA AAGCTCTCTG AAGATGAAGC AAAAGTTGCA |
| | GACAACCTCA |
| 1301 | CTTCTACGCT GAAGCAGCCP GTAACTCTAA CTGCAGGAAA |
| | TTTAGTACTT |
| 1351 | AAACGTGGTG TCACTCTCGA TACGAAAGGC TTTACTCAGA |
| | CCGCGGGTTC |
| 1401 | CTCTGTTATT ATGGATGCGG GCACAACGTT AAAAGCAAGT |
| | ACAGAGGAGG |
| 1451 | TCACTTTAAC AGGTCTTTCC ATTCCTGTAG ACTCTTTAGG |
| | CGAGGGTAAG |
| 1501 | AAAGTTGTAA TTGCTGCTTC TGCAGCAAGT AAAAATGTAG |
| | CCCTTAGTGG |
| 1551 | TCCGATTCTT CTTTTGGATA ACCAAGGGAA TGCTTATGAA |
| | AATCACGACT |
| 1601 | TAGGAAAAAC TCAAGACTTT TCATTTGTGC AGCTCTCTGC |
| | TCTGGGTACT |
| 1651 | GCAACAACTA CAGATGTTCC AGCGGTTCCT ACAGTAGCAA |
| | CTCCTACGCA |
| 1701 | CTATGGGTAT CAAGGTACTT GGGGAATGAC TTGGGTTGAT |
| | GATACCGCAA |
| 1751 | GCACTCCAAA GACTAAGACA GCGACATTAG CTTGGACCAA |
| | TACAGGCTAC |
| 1801 | CTTCCGAATC CTGAGCGTCA AGGACCTTTA GTTCCTAATA |
| | GCCTTTGGGG |
| 1851 | ATCTTTTTCA GACATCCAAG CGATTCAAGG TGTCATAQAG |
| | AGAAGTGCTT |
| 1901 | TGACTCTTTG TTCAGATCGA GGCTTCTGGG CTGCGGGAGT |
| | CGCCAATTTC |
| 1951 | TTAGATAAAG ATAAGAAAGG GGAAAAACGC AAATACCGTC |
| | ATAAATCTGG |
| 2001 | TGGATATGCT ATCGGAGGTG CAGCGCAAAC TTGTTCTGAA |
| | AACTTAATTA |
| 2051 | GCTTTGCCTT TTGCCAACTC TTTGGTAGCG ATAAAGATTT |
| | CTTAGTCGCT |
| 2101 | AAAAATCATA CTGATACCTA TGCAGGAGCC TTCTATATCC |
| | AACACATTAC |
| 2151 | AGAATGTAGT GGGTTCATAG GTTGTCTCTT AGATAAACTT |
| | CCTCGCTCTT |
| 2201 | GGAGTCATAA ACCCCTCGTT TTAGAAGGGC AGCTCGCTTA |
| | TAGCCACGTC |
| 2251 | AGTAATGATC TGAAGACAAA GTATACTGCG TATCCTGAGG |
| | TGAAAGGTTC |
| 2301 | TTGGGGGGAT AATGCTTTTA ACATGATGTT GGGAGCTTCT |
| | TCTCATTCTT |
| 2351 | ATCCTGAATA CCTGCATTGT TTTGATACCT ATCCTCCATA |
| | CATCAAACTG |
| 2401 | AATCTGACCT ATATACGTCA GGACAGCTTC TCGGAGAAAG |
| | GTACAGAAGG |
| 2451 | AAGATCTTTT GATGACAGCA ACCTCTTCAA TTTATCTTTG |
| | CCTATAGGGG |
| 2501 | TGAAGTTTGA GAAGTTCTCT GATTGTTATG ACTTTTCTTA |
| | TGATCTGACT |
| 2551 | TTATCCTATG TTCCTGATCT TATCCGCAAT GATCCCAAAT |
| | GCACTACAGC |
| 2601 | ACTTGTAATC AGCGGAGCCT CTTGGGAAAC TTATGCCAAT |
| | AACTTAGCAC |
| 2651 | GACAGGCCTT GCAAGTGCGT GCAGGCAGTC ACTACGCCTT |
| | CTCTCCTATG |
| 2701 | TTTGAAGTGC TCGGCCAGTT TGTCTTTGAA GTTCGTGGAT |
| | CCTCACGGAT |

The PSORT algorithm predicts an outer membrane location (0.922).

The protein was expressed in E. coli and purified as a GST-fusion product, as shown in FIG. 48A. The recombinant protein was used to immunise mice, whose sera were used in a Western blot (FIG. 48B) and for FACS analysis (FIG. 48C). A his-tagged protein was also expressed.

The cp0010 protein was also identified in the 2D-PAGE experiment and showed good cross-reactivity with human sera, including sera from patients with pneumonitis.

These experiments show that cp0010 is a surface-exposed and immunoaccessible protein, and that it is a useful immunogen. These properties are not evident from the sequence alone.

### Example 49

The following C. pneumoniae protein (PID 4376296) was expressed <SEQ ID 97; cp6296>:

| | |
|---|---|
| 1 | MEEVSEYLQQ VENQLRSCSK RLTKMETFAL GVRLEAKEEI |
| | ESIILSDVVN |
| 51 | RFEVLCRDIE DMLSRVEEIE RMLHMAELPL LPIKRALTKA |
| | EVQHNSCKEK |
| 101 | LTKVEPYFKE SPAYLTSEER LQSLNQTLQR AYKESQKVSG |
| | LESEVRACRB |
| 151 | QLKDQVRQFE TQGVSLIKEE ILFVTSTFRT KFSYHSFRLH |
| | VPCMRLYEEY |
| 201 | YDDIDLERTR ARWMAMSERY RDAFQAPQEM LKEGLVEEAQ |
| | ALRBTEYWLY |
| 251 | REERKSKKKH* |

The cp6296 nucleotide sequence <SEQ ID 98> is:

| | |
|---|---|
| 1 | ATGGAGGAGG TGTCTGAGTA TCTTCAGCAA GTAGAAAATC |
| | AGTTGGAATC |
| 51 | CTGTTCCAAG CGATTAACCA AGATGGAAAC TTTTGCCTTA |
| | GGTGTGAGGT |
| 101 | TGGAAGCTAA AGAAGAGATA GAGTCTATCA TACTTTCTGA |
| | TGTAGTGAAC |
| 151 | CGTTTTGAGG TTTTATGTAG AGATATTGAA GATATGCTAT |
| | CTCGAGTCGA |
| 201 | GGAGATAGAG CGGATGTTAC GTATGGCGGA GCTTCCTCTA |
| | CTTCCTATAA |
| 251 | AAGAAGCGCT TACCAAGGCT TTTGTACAAC ATAACAGCTG |
| | TAAAGAGAAG |
| 301 | TTAACCAAGG TAGAGCCTTA CTTTAAAGAG AGCCCTGCAT |
| | ATCTAACTAG |
| 351 | TGAAGAGCGA TTGCAGAGTT TGAATCAGAC TTTACAACGT |
| | GCGTACAAAG |
| 401 | AGTCCCAAAA GGTTTCAGGT TTAGAATCGG AAGTGAGAGC |
| | CTGTCGAGAG |
| 451 | CAGCTTAAAG ATCAAGTAAG ACAGTTTGAA ACTCAAGGAG |
| | TGAGCTTGAT |
| 501 | AAAAGAAGAG ATTCTCTTTG TGACTAGTAC CTTTAGAACT |
| | AAATTTAGCT |
| 551 | ATCATTCATT TCGATTACAT GTTCCTTGCA TGAGGTTGTA |
| | TGAGGAGTAT |
| 601 | TATGATGACA TTGATCTAGA GAGAACTCGA GCTCGATGGA |
| | TGGCGATGTC |
| 651 | TGAGAGGTAT AGAGATGCTT TTCAGGCATT CCAGGAGATG |
| | TTGAAGGAAG |
| 701 | GCCTAGTTGA AGAAGCTCAG GCTCTTTAGG AAACCGAGTA |
| | CTGGTTATAT |
| 751 | CGAGAGGAGA GAAAGAGTAA AAAGAAACAT TGA |

The PSORT algorithm predicts a cytoplasmic location (0.523).

The protein was expressed in E. coli and purified as a GST-fusion product, as shown in FIG. 49A. The recombinant protein was used to immunise rice, whose sera were used in a Western blot (FIG. 49B) and for FACS analysis (FIG. 49C). A his-tagged protein was also expressed.

These experiments show that cp6296 is a surface-exposed and immunoaccessible protein, and that it is a useful immunogen. These properties are not evident from the sequence alone.

### Example 50

The following C. pneumoniae protein (PID 4376664) was expressed <SEQ ID 99; cp6664>:

| | |
|---|---|
| 1 | MVLFHAQASG RWRVKADAIV LPFWHFKDAK NAASFEAEFE |
| | PSYLPALENF |
| 51 | QGKTGEIELL YSSPKMCEKR IVLLGLGKNE ELYDFBBGWY |
| | YATLTRVLRK |
| 101 | AKCSTVNIIL PTISELRLSA EEFLVGLSSG ILSLNYDYPR |
| | YNKVDRNLET |
| 151 | PLSKVTVIGI VPKMADAIFR KEAAIFEGVY LTRDLVNRNA |
| | DEITPKKLAE |
| 201 | VALNLGKEFP SIDTKVLGKD AIAKEKMGLL LAVSKGSCVD |
| | PHFIVVRYQG |
| 251 | RPKSKDHTVL IGKGVTFDSG GLDLKPGKSM LTMKEDMAGG |
| | ATVLGILSAL |
| 301 | AVLELPINVT GIIPATENAI DGASYKMGDV YVGNSGLSVE |
| | ICSTDAEGRL |
| 351 | ILADAITTAL KYCKPTRIID FATLTGAMVV SLGEEVAGFF |
| | SNNDVLAEDL |
| 401 | LEASAETSEP LWRLPLVKKY DKTLHSDIAD MENLGSNRAG |
| | AITAALFLQR |
| 451 | FLEESSVAWA HLDIAGTAYH EKEEDRYPKY ASGFCVRSIL |
| | YYLENSLSK* |

The cp6664 nucleotide sequence <SEQ ID 100> is:

| | |
|---|---|
| 1 | GTGGTTTTAT TTCATGCTCA AGCCTCTGGG CGTAATCGTG |
| | TTAAGGCAGA |
| 51 | TGCTATAGTC CTGCCCTTTT GGCATTTTAA GGATGCAAAA |
| | AATGCAGCTT |
| 101 | CTTTTGAAGC CGAGTTTGAA CCCTCGTATC TCCCCGCTTT |
| | AGAAAACTTT |
| 151 | CAAGGAAAAA CCGGGGAGAT TGAACTCCTT TATAGTAGTC |
| | CTAAAGCTAA |
| 201 | GGAAAAACGC ATTGTCCTCT TAGGCTTAGG GAAAAATGAA |
| | GAGCTCACCT |
| 251 | CTGATGTTGT TTTCCAAACc TATGCGACAC TAACTCGTGT |
| | CTTACGTAAA |
| 301 | GCAAAGTGTT CCACAGTCAA TATCATCTTA CCTACAATTT |
| | CTGAATTGCC |
| 351 | GCTTTCTGCC GAAGAATTCT TAGTGGGGTT GTCCTCAGGA |
| | ATTTTGTCAT |
| 401 | TAAACTATGA CTACCCACGT TATAATAAGG TAGATCGTAA |
| | TCTTGAAACT |
| 451 | CCTCTTTCTA AAGTCACGGT TATCGGTATC GTTCCCAAAA |
| | TGGCGGATGC |
| 501 | TATCTTTAGG AAAGAAGCAG CCATTTTCGA AGGCGTATAT |
| | CTCACTCGAG |
| 551 | ATCTTGTGAA CAGGAATGCT GATGAAATTA CCCCTAAGAA |
| | ATTGGCAGAG |
| 601 | GTTGCTCTGA ATCTGGGAAA AGAGTTCCCT AGTATTGATA |
| | CTAAGGTCTT |
| 651 | GGGAAAAGAT GCCATCGCCA AAGAGAAAAT GGCACTCCTA |
| | TTGGCTGGTT |
| 701 | CCAAGGGTTC TTGTGTGGAT CCACACTTTA TCGTTGTCCG |
| | TTATCAAGGA |
| 751 | CGTCCTAAGT CTAAAGATCA CACCGTCTTG ATAGGGAAAG |
| | GGGTCACTTT |
| 801 | TGACTCTGGA GGTTTAGACC TCAAGCCTGG AAAATCCATG |
| | CTTACTATGA |
| 851 | AAGAAGACAT GGCAGGTGGG GCTACAGTCC TCGGGATTCT |
| | CTCGGCGTTA |
| 901 | GCAGTTTTAG AGCTTCCTAT AAATGTCACG GGGATCATTC |
| | CTGCTACAGA |
| 951 | GAATGCTATC GATGGCGCCT CCTATAAAAT GGGAGATGTC |
| | TATGTAGGAA |
| 1001 | TGTCGGGGCT TTCTGTTGAG ATTTGTAGTA CCGATGCTGA |
| | GGGACGTCTT |
| 1051 | ATCCTCGCTG ATGCGATTAC ATATGCTTTA AAATATTGTA |
| | AACCGACACG |
| 1101 | TATTATAGAT TTTGCAACTC TAACAGGAGC TATGGTAGTC |
| | TCTCTAGGAG |
| 1151 | AAGAGGTTGC AGGTTTCTTT TCCAATAACG ATGTTTTAGC |
| | TGAAGATCTT |
| 1201 | TTAGAGGCGT CAGCCGAAAC CTCCGAGCCG TTATGGAGAC |
| | TTCCTCTAGT |
| 1251 | TAAGAAGTAT GATAAAACAT TGCATTCTGA TATTGCTGAT |
| | ATGAAAAATC |
| 1301 | TAGGCAGTAA CCGTGCAGGG OCTATTACAG CAGCATTATT |
| | CTTGCAGAGA |
| 1351 | TTTTTGGAAG AATCTTCGGT AGCTTGGGCA CATCTTGATA |
| | TTGCAGGTAC |
| 1401 | TGCATATCAT GAAAAAGAAG AAGACCGTTA TCCAAAATAT |
| | GCTTCAGGTT |
| 1451 | TTGGTGTTCG TTCTATTCTT TATTACTTAG AAAATAGTCT |
| | TTCTAAGTAG |

The PSORT algorithm predicts an inner membrane location (0.268).

The protein was expressed in E. coli and purified as a GST-fusion (FIG. 50A), as a his-tagged protein, and as a GST/His fusion. The proteins were used to immunise mice, whose sera were used in Western blot Western blot (50B) and FACS (50C) analyses.

The cp6664 protein was also identified in the 2D-PAGE experiment (Cpn0385) and showed good cross-reactivity with human sera, including sera from patients with pneumonitis.

These experiments show that cp6664 is a surface-exposed and immunoaccessible protein, and that it is a useful immunogen. These properties are not evident from the sequence alone.

### Example 51

The following C. pneumoniae protein (PID 4376696) was expressed <SEQ ID 101; cp6696>:

| | |
|---|---|
| 1 | MTLIFVIIIV WCNAFLIKLC VIMGLQSRLQ HCIEVSQNSN |
| | FDSQVKQFIY |
| 51 | ACQDKTLRQS VLKIFRYHPL LRIHDIAEAV YLLMALEEGB |
| | DLGLSFLNVQ |
| 101 | QYPSGAVELP SCGGFPWKGL PYPAEHAEFG LLLLQIAEPY |
| | EESQAYVSKM |
| 151 | SHPQQALFDH QGSVFPSLWS QENSRLLKEK TTLSQSFLFQ |
| | LGMQIHPBYS |
| 201 | LEDPALGFWM QRTRSSSAFV AASGCQSSLG AYSSGDVGVI |
| | AYGPCSGDIS |
| 251 | DCYYFGCCGI AREFVCQKSH QTTEISFLTS TGKPIWRNTG |
| | FSYLRDSYVH |
| 301 | LPIRCRITIS DKQYRVHAAL AEATSAMTFS IFCKGKNCQV |
| | VDGPRLRSCS |
| 351 | LDSYXGPGND IMILGENDAI NIVSASPYME IFALQGKEKF |
| | WNADFLINIP |
| 401 | YKEEGVMLIF EKKVTSEKGR FFTKMN* |

A predicted signal peptide is highlighted.

The cp6696 nucleotide sequence <SEQ ID 102> is:

| | |
|---|---|
| 1 | TTGACTCTAA TTTTTGTTAT TATTATCGTT TGGTGCAATG |
| | CTTTTCTGAT |
| 51 | CAAATTGTGC GTGATAATGG GGCTGCAATC CAGGTTACAA |
| | CATTGTATAG |
| 101 | AAGTGTCCCA GAATTCGAAC TTTGATTCAC AAGTAAAACA |
| | GTTPATCTAT |
| 151 | GCGTGCCAAG ATAAGACATT AAGGCAGTCT GTACTCAAGA |
| | TTTTCCGCTA |
| 201 | CCATCCTTTA CTAAAAATTC ATGATATTGC TCGGGCCGTC |
| | TATCTTTTGA |
| 251 | TGGCCTTAGA AGAAGGCGAG GATTTAGGCT TAAGCTTTTT |
| | AAATGTACAG |
| 301 | CAGTACCCTT CAGGTGCTGT AGAACTGTTT TCTTGTGGGG |
| | GATTTCCTTG |
| 351 | GAAAGGATTA CCTTATCCTC CAGAACATGC GGAATTTGGC |
| | CTACTCCTGT |
| 401 | TACAGATCGC AGAGTTTTAT GAAGAGAGTC AGGCATACGT |
| | CTCTAAAATG |
| 451 | AGTCATTTTC AACAGGCACT CTTTGATCAC CAAGGGAGCG |
| | TCTTTCCCTC |
| 501 | TCTCTGGAGC CAGGAGAACT CTCGACTCCT AAAAGAAAAG |
| | ACAACTCTTA |
| 551 | GCCAATCGTT TCTCTTCCAA TTAGGAATGC AAATTCACCC |
| | AGAATACAGT |
| 601 | CTTGAGGATC CTGCACTAGG GTTCTGGATG CAAAGAACGC |
| | GTTCTTCATC |
| 651 | CGCTTTTGTA GCCGCTTCAG GATGTCAAAG TAGCTTGGGA |
| | GCGTATTCCT |
| 701 | CAGGGGATGT CGGTGTTATC GCTTATGGAC CTTGCTCTGG |
| | AGACATTAGT |
| 751 | GATTGTTATT ATTTTGGATG TTGTGGAATC GCTAAAGAGT |
| | TCGTGTGCCA |
| 801 | AAAATCTCAC CAAACTACAG AGATTTCTTT TCTCACCTCT |
| | ACAGGAAAGC |
| 851 | CTCATCCCAG AAATACGGGA TTTTCCTACC TTCGAGATTC |
| | CTATGTACAT |
| 901 | CTGCCGATCC GCTGTAAGAT CACTATTTCC GACAAGCAAT |
| | ATCGCGTGCA |
| 951 | CGCTGCGTTG GCTGAGGCCA CCTCTGCCAT GACGTTTTCT |
| | ATTTTCTGTA |
| 1001 | AGGGGAAGAA TTGTCAGGTT GTTGACGGCC CTCGCTTGCG |
| | CTCCTGTTCC |
| 1051 | CTAGATTCTT ATAAAGGTCC CGGAAACGAC ATTATGATTC |
| | TTGGGGAAAA |
| 1101 | TGACGCAATC AACATTGTTT CTGCAAGTCC CTATATGGAA |
| | ATTTTTGCTT |
| 1151 | TGCAAGGCAA AGAAAAATTT TGGAATGCAG ACTTTTTGAT |
| | TAATATTCCT |
| 1201 | TACAAAGAAG AGGGCGTCAT GTTAATTTTT GAAAAAAAAG |
| | TGACCTCTGA |
| 1251 | GAAAGGAAGA TTCTTTACGA AGATGAATTA A |

The PSORT algorithm predicts an inner membrane location (0.463).

The protein was expressed in E. coli and purified as a GST-fusion product, as shown in FIG. 51A. The recombinant protein was used to immunise nice, whose sera were used in a Western blot (FIG. 51B) and for FACS analysis (FIG. 51C). A his-tagged protein was also expressed.

This protein also showed good cross-reactivity with human sera, including sera from patients with pneumonitis.

These experiments show that cp6696 is a surface-exposed and immunoaccessible protein, and that it is a useful immunogen. These properties are not evident from the sequence alone.

### Example 52

The following C. pneumoniae protein (PID 4376790) was expressed <SEQ ID 103; cp6790>:

| | |
|---|---|
| 1 | MSEHXKSSKI IGIDLGTTNS CVSVMEGGQA KVITSSEGTR |
| | TTPSIVAFKG |
| 51 | NEKLVGIPAK RQAVTNPEKT LGSTKRFIGR KYSEVASEIQ |
| | TVPYTVTSGS |
| 101 | KGDAVFEVDG KQYTPEBIGA QILNXMKETA EAYLGETVTE |
| | AVITVPAYFN |
| 151 | DSQRASTRDA GRIAGLDVKR IIPEPTAAAL AYGIDKVGDK |
| | KIAVFDLGGG |
| 201 | TFDISILEIG DGVFEVLSTN GDTLLGGDDF DEVIIKWMIE |
| | EFKKQEGIDL |
| 251 | SKDNMALQRL KDAAEKAXIE LSGVSSTEIN QPFITMDAQG |
| | PKHLALTLTR |
| 301 | AQFEKLAASL IERTKSPCIK ALSDAKLSAK DIDDVLLVGG |
| | MSRMPAVQET |
| 351 | VKELPGKEPN KGVNPDEVVA IGAAIQGGVL GGEVKDVLLL |
| | DVIPLSLGIE |
| 401 | TLGGVMTTLV ERNTTIPTQK KQIFSTAADN QPAVTIVVLQ |
| | GERPMAKDNK |
| 451 | EIGRFDLTDI PPAPRGHPQI EVSFDIDANG IPHVSAKDVA |
| | SGKEQKIRIE |
| 501 | ASSGLQEDEI QRMVRDAEIN KEEDKKRREA SDAKNEADSM |
| | IFRAEKAIKD |
| 551 | YKEQIPETLV KEIEERIENV RNALKDDAPI EKIKEVTEDL |
| | SKHMQKIGES |
| 601 | MQSQSASAAA SSAANAKGGP NINTEDLKKH SFSTKPPSNN |
| | GSSEDHIEEA |
| 651 | DVEIIDNDDK* |

The cp6790 nucleotide sequence <SEQ ID 104> is:

| | |
|---|---|
| 1 | ATGAGTGAAC ACAAAAAATC AAGCAAAATT ATAGGTATAG |
| | ACTTAGGCAC |
| 51 | AACAAACTCC TGCGTATCTG TTATGGAAGG AGGACAAGCT |
| | AAAGTAATTA |
| 101 | CATCATCCGA AGGAACAAGA ACCACGCCAT CGATCGTTGC |
| | CTTCAAAGGT |
| 151 | AATGAGAAAT TAGTGGGGAT TCCAGCAAAA CGTCAAGCAG |
| | TGACAAATCC |
| 201 | AGAAAAAACT CTCGGCTCTA CAAAACGCTT TATTGGCCGT |
| | AAGTACTCTG |
| 251 | AAGTAGCTTC GGAAATCCAA ACCGTTCCTT ATACAGTCAC |
| | CTCCGGATCT |
| 301 | AAAGGTGATG CCGTTTTCCA AGTTGATGGC AAACAATACA |
| | CTCCAGAAGA |
| 351 | AATTGGCGCA CAAATCTTAA TGAAAATGAA AGAGACACCA |
| | GAAGCTTATC |
| 401 | TAGGCGAAAC TGTCACAGAA GCAGTGATCA CCGTCCCCGC |
| | ATACTTCAAT |
| 451 | GATTCTCAAC GAGCATCCAC AAAAGATGCT GGACGCATTG |
| | CAGGTCTAGA |
| 501 | TGTAAAACGT ATCATTCCAG AACCTACCGC AGCAGCTCTT |
| | GCCTACGGAA |
| 551 | TCGATAAAGT CGGTGATAAA AAAATCGCTG TCTTCCACCT |
| | TGGTGGAGGA |
| 601 | ACTTTTGATA TCTCCATCCT AGAAATCGGT GATGGCGTCT |
| | TCGAAGTTCT |
| 651 | ATCTACAAAT GGAGATACTC TCCTCGGTGG AGACGACTTT |
| | GATGAAGTCA |
| 701 | TTATCAAATG GATGATCGAA GAATTCAAAA AACAAGAAGG |
| | CATTGATCTT |
| 751 | AGCAAAGATA ATATGGCCTT ACAAAGACTT AAAGATGCTG |
| | CTGAGAAAGC |
| 801 | AAAAATAGAA CTTTCAGGAG TCTCTTCCAC AGAAATCAAT |
| | CAGCCATTCA |
| 851 | TCACAATGGA TGCACAAGGA CCTAAACACC TTGCATTGAC |
| | ACTCACACGT |
| 901 | GCGCAATTCG AGAAACTCGC AGCCTCTCTA ATCGAAAGAA |
| | CAAAATCTCC |
| 951 | ATGCATCAAA GCACTCAGTG ACGCAAAACT TTCCGCTAAG |
| | GATATCGATG |
| 1001 | ATGTTCTCTT AGTTGGAGGT ATGTCAAGAA TGCCCGCAGT |
| | GCAAGAAACT |
| 1051 | GTAAAAGAAC TCTTCGGCAA AGAGCCTAAT AAAGGAGTCA |
| | ACCCCGACGA |
| 1101 | AGTTGTTGCT ATTGGAGCCG CAATTCAAGG TGGTGTTCTT |
| | GGCGGAGAAG |
| 1151 | TTAAGGATGT TCTACTTCTA GACGTTATCC CCCTATCTCT |
| | GGGTATCGAA |
| 1201 | ACTCTAGGAG GCGTCATGAC GACTCTGGTA GAGAGAAATA |
| | CTACAATCCC |
| 1251 | TACACAGAAA AAACAAATCT TCTCCACAGC TGCTGATAAC |
| | CAGCCTGCGG |
| 1301 | TTACCATCGT AGTTCTCCAA GGAGAGCGTC CCATGGCCAA |
| | AGATAACAAG |
| 1351 | GAAATCGGAA GATTCGATCT TACAGATATC CCTCCGGCTC |
| | CTCGAGGCCA |
| 1401 | TCCTCAAATC GAAGTCTCCT TCGATATCGA TGCAAACGGA |
| | ATTTTCCATG |
| 1451 | TCTCAGCTAA AGATGTTGCC AGCGGTAAAG AACAGAAAAT |
| | TCGTATCGAA |
| 1501 | GCAAGCTCAG GACTTCAAGA AGATGAAATC CAAAGAATGG |
| | TTCGAGATGC |
| 1551 | CGAAATTAAT AAGGAAGAAG ATAAAAAACG TCGPGAAGCT |
| | TCAGATGCTA |
| 1601 | AAAATGAAGC CGATAGCATG ATCTTCAGAG CCGAAAAAGC |
| | TATTAAAGAT |
| 1651 | TATAAGGAGC AAATTCCTGA AACTTTAGTT AAAGAAATCG |
| | AAGAGCGAAT |
| 1701 | CGAAAACGTG CGCAACGCAC TCAAAGATGA CGCTCCTATT |
| | GAAAAAATTA |
| 1751 | AAGAGGTTAC TGAAGACCTA AGCAAGCATA TGCAAAAAAT |
| | TGGAGAGTCT |
| 1801 | ATGCAATCGC AGTCTGCATC AGCAGCAGCA TCATCGGCAG |
| | CCAATGCTAA |
| 1851 | AGGTGGACCT AACATCAATA CAGAAGATTT GAAAAAACAT |
| | AGTTTCAGTA |
| 1901 | CGAAGCCTCC TTCAAATAAC GGTTCTTCAG AAGACCATAT |
| | CGAAGAAGCT |
| 1951 | GATGTAGAAA TTATTGATAA CGACGATAAG TAA |

The PSORT algorithm predicts an inner membrane-location (0.151).

The protein was expressed in E. coli and purified as a GST-fusion product (FIG. 52A) and a his-tagged product. The proteins were used to immunise mice, whose sera were used in Western blot (FIG. 52B) and FACS (FIG. 52C) analyses.

The cp6790 protein was also identified in the 2D-PAGE experiment (Cpn0503).

These experiments show that cp6790 is a surface-exposed and immunoaccessible protein, and that it is a useful immunogen. These properties are not evident from the sequence alone.

### Example 53

The following C. pneumoniae protein (PID 4376878) was expressed <SEQ ID 105; cp6878>:

| | |
|---|---|
| 1 | MNVPDSKNLH PPAYELLEIK ARITQSYKEA SAILTAIPDG |
| | ILLLSETGHF |
| 51 | LICNSQAREI LGIDENLEIL NRSFTDVLPD TCLGFSIQEA |
| | LESLKVPKTL |
| 101 | RLSLCKESKE KEVELFIRKN EISGYLFIQI RDRSDYKQLE |
| | NAIERYKNIA |
| 151 | ELGKMTATLA HEIRNPLSGI VGFASILKKE ISSPRHORML |
| | SSIISGTRSL |
| 251 | LFRSIDPDRM NSVVWNLVKN AVETGNSPIT LTLHTSGDIS |
| | VTNPGTIPSE |
| 301 | IMDRLFTPFF TTKREGNGLC LAEAQKIIRL HGGDIQLKTS |
| | DSAVSFFIII |
| 351 | PELLAALFKE RAAS* |

The cp6878 nucleotide sequence <SEQ ID 106> is:

| | |
|---|---|
| 1 | ATGAACGTCC CTGATTCCAA GAACCTCCAT CCTCCTGCAT |
| | ACGAACTCCT |
| 51 | AGAGATCAAG GCTCGCATCA CACAATCTTA TAAAGAAGCC |
| | AGTGCTATAC |
| 101 | TGACAGCGAT TCCTGATGGT ATCCTATTAC TTTCTGAAAC |
| | AGGACACTTT |
| 151 | CTTATCTGCA ATTCACAAGC ACGTGAAATT CTAGGAATTG |
| | ATGAAAATCT |
| 201 | AGAAATTCTT AATAGATCCT TTACCGATGT TCTCCCCGAT |
| | ACGTGTCTTG |
| 251 | GATTTTCTAT TCAAGAGGCT CTTGAATCTC TAAAAGTCCC |
| | TAAAACTCTT |
| 301 | AGACTCTCTC TCTGTAAAGA ATCTAAAGAA AAAGAAGTGG |
| | AACTCTTCAT |
| 351 | CCGTAAAAAC GAOATCAGTG GATACCTGTT TATCCAAATC |
| | CGCGATCGGT |
| 401 | CCGACTATAA ACAACTAGAA AACGCTATAG AAAGATATAA |
| | AAATATCGCA |
| 451 | GAACTTGGGA AAATGACGGC TACCCTAGCT CACGAAATCC |
| | GCAATCCGCT |
| 501 | AAGTGGAATC GTTGGATTTG CCTCTATCCT AAAQAAAGAG |
| | ATTTCCCCTC |
| 551 | CTCGCCACCA ACGAATGCTC TCCTCAATCA TCTCCGGCAC |
| | AAGGTCTCTA |
| 601 | AATAACCTTG TCTCTTCTAT GTTAGAATAT ACAAAATCAC |
| | AACCGTTGAA |
| 651 | CCTAAAGATT ATAAATTTAC AAGACTTCTT CTCTTCTCTT |
| | ATCCCTCTGC |
| 701 | TCTCCGTCTC TTTCCCGAAT TGCAAGTTTG TAAGAGAGGG |
| | CGCACAACCT |
| 751 | CTATTCAGAT CTATAGATCC TGATCGGATG AACAGTGTCG |
| | TTTGGAACCT |
| 801 | AGTGAAAAAT GCTGTAGAAA CAGGGAACTC TCCGATCACT |
| | CTGACCCTGC |
| 851 | ATACATCGGG AGACATCTCG GTAACGAACC CCGGAACGAT |
| | TCCTTCCGAG |
| 901 | ATCATGGACA AGCTCTTCAC TCCATTCTTC ACAACAAAGA |
| | GAGAGGGAAA |
| 951 | TGGTTTGGGA CTTGCTGAAG CTCAAAAAAT TATAAGACTC |
| | CATGGAGGAG |
| 1001 | ATATCCAATT AAAAACAAGC GACTCCGCCG TTAGCTTCTT |
| | CATAATCATC |
| 1051 | CCCGAACTTC TAGCGGCCCT ACCCAAAGAA AGAGCCGCTA G |

The PSORT algorithm predicts an inner membrane location (0.204).

The protein was expressed in E. coli and purified as a his-tag product (FIG. 53A) and as a GST-fusion product. The recombinant GST-fusion protein was used to immunise mice, whose sera were used in a Western blot (FIG. 53B) and for FACS analysis.

These experiments show that cp6878 is a surface-exposed and immunoaccessible protein, and that it is a useful immunogen. These properties are not evident from the sequence alone.

### Example 54

The following C. pneumoniae protein (PID 4377224) was expressed <SEQ ID 107; cp7224>:

| | |
|---|---|
| 1 | MMKKIRKVAL AVGGSGGHIV PALSVKEAFS REGIDVLLLG |
| | KGLKNHPSLQ |
| 51 | QGISYREIPS GLPTVUIPIK IMSRTLSLCS GYLKARKELK |
| | IFDPDLVIGF |
| 101 | GSYUSLPVLL AGLSHKIPLP LHEQNLVPGK VNQLFSRYAR |
| | GIGVNFSPVT |
| 151 | KEFRCPAEEV FLPKRSFSLG SPMMKRCTNH TPTICVVGGS |
| | QGAQILNTCV |
| 201 | PQALVKLVNK YPNMYVHHIV GPKSDVMKVQ EVYNRGEVLC |
| | CVKPDEEQLL |
| 251 | DVLLAADLVI SRAGATILEE ILWAKVPGIL IPYPGAYGHQ |
| | EVNAKFFVDV |
| 301 | LEGGTNILEK ELTEKLLVEK VTFALDSHNR EKQRNSLAAY |
| | SQQRSTKTFH |

The cp7224 nucleotide sequence <SEQ ID 108> is:

| | |
|---|---|
| 1 | ATGATGAAGA AAATTCGAAA AGTAGCCTTG GCTGTAGGAG |
| | GTTCAGGAGG |
| 51 | CCACATTGTC CCAGCTCTCT CGGTAAAGGA AGCTTTTTCT |
| | CGTGAAGGAA |
| 101 | TAGACGTATT ACTACTAGGG AAAGGTCTCA AGAACCATCC |
| | TTCTTTGCAA |
| 151 | CAGGGAATCA GCTATCGGGA AATCCCCTCA GGACTTCCTA |
| | CAGTCCTTAA |
| 201 | TCCCATAAAG ATCATGAGCA GGACCCTTTC TCTATGTTCA |
| | GGATACCTGA |
| 251 | AAGCAAGAAA GGAACTTAAA ATTTTTGACC CTGACCTGGT |
| | CATAGGATTT |
| 301 | GGGAGCTACC ACTCTCTTCC CGTQTTGCTC GCAGGACTGT |
| | CCCATAAAAT |
| 351 | TCCCTTATTT CTACACGAAC AAAATCTAGT TCCTGGAAAA |
| | GTAAATCAAT |
| 401 | TGTTTTCCCG CTATGCTCGA GGTATTGGAG TGAATTTCTC |
| | CCCCGTTACT |
| 451 | AAACACTTCC GCTGCCCCGC AGAAGAGGTC TTCCTTCCTA |
| | AACGAAGCTT |
| 501 | CTCCTTAGGA AGCCCTATGA TGAAGCGATG TACAAATCAT |
| | ACCCCTACAA |
| 551 | TCTGTCTTGT TGGAGGTTCT CAGGGAGCAC AGATATTAAA |
| | TACTTGTGTT |
| 601 | CCCCAAGCTC TTGTCAAGCT AGTCAATAAG TACCCAAATA |
| | TGTACGTCCA |
| 651 | TCATATTGTA GGACCTAAAA GTGATGTTAT GAAGGTGCAA |
| | CATGTTTACA |
| 701 | ATCGTGGAGA GGTCCTCTGC TGTGTGAAGC CGTTCGAAGA |
| | GCAACTCCTA |
| 751 | GATGTCTTGC TTGCCGCAGA TTTGGTCATC AGTAGGGCAG |
| | GAGCCACAAT |
| 801 | TTTAGAAGAA ATTCTTTGGG CAAAAGTTCC CGGAATTTTA |
| | ATTCCCTATC |
| 851 | CAGGAGCTTA TGGACATCAG GAAGTTAATG CTAAATTCTT |
| | TGTAGACGTC |
| 901 | TTAGAAGGGG GAACTATGAT CCTAGAAAAA GAATTAACAG |
| | AGAAGCTATT |
| 951 | AGTAGAAAAA GTAACGTTTG CTTTAGACTC CCATAACAGA |
| | GAAAAACAAC |
| 1001 | GCAATTCCCT AGCGGCGTAT AGTCAGCAAA GGTCAACAAA |
| | AACATTCCAT |
| 1051 | GCATTCATTT GTGAATGCTT ATAG |

The PSORT algorithm predicts an inner membrane location (0.164).

The protein was expressed in E. coli and purified as a GST-fusion product, as shown in FIG. 54A. The recombinant protein was used to immunise mice, whose sera were used in a Western blot (FIG. 54B) and for FACS analysis (FIG. 54C). A his-tagged protein was also expressed.

This protein also showed good cross-reactivity with human sera, including sera from patients with pneumonitis.

These experiments show that cp7224 is a surface-exposed and immunoaccessible protein, and that it is a useful immunogen. These properties are not evident from the sequence alone.

### Example 55

The following C. pneumoniae protein (PID 4377140) was expressed <SEQ ID 109; cp7140>:

| | |
|---|---|
| 1 | MVRRSISFCL FFLMTLLCCT SCNSRSLIVH GLFGREANEI |
| | VVLLVSKGVA |
| 51 | AQKLPQAAAATAGAATEQMW DIAVPSAQIT EALAILNQAG |
| | LPRMKGTSLL |
| 101 | DLFAKQGLVP SELQEKIRYQ EGLSEQMAST IRKMDGVVDA |
| | SVQISFTTEN |
| 151 | EDNLPLTASV YIKHRGVLDN PNSIMVSKIK RLIASAVPGL |
| | VPENVSVVSD |
| 201 | HAAYSDITIN GPWGLTEEID YVSVWGIILA KSSLTXFRLI |
| | FYVLILILFV |
| 251 | ISCGLLWVIW KTHTLIMTMG GTKGFFNPTP YTKNALEAKK |
| | AEGAAADKEK |
| 301 | KEDADSQGES KNAETSKDKS SDKDAPEGSN EIEGA* |

A predicted signal peptide is highlighted.

The cp7140 nucleotide sequence <SEQ ID 110> is:

| | |
|---|---|
| 1 | ATGGTTCGTC GATCTATTTC TTTTTGCTTG TTCTTTCTAA |
| | TGACATTGCT |
| 51 | GTGCTGTACA AGCTGThACA GCAGGTCTCT AATTGTGCAC |
| | GGTCTTCCTG |
| 101 | GCAGAGAAGC GAATGAGATT GTGGTGCTTT TGGTAAGCAA |
| | AGGGGTGGCT |
| 151 | GCACAAAAAT TGCCTCAAGC TGCAGCGGCT ACAGCCGGAG |
| | CAGCTACTGA |
| 201 | GCAAATGTGG GATATCGCGG TTCCGTCAGC ACAAATCAAA |
| | GAGGCCCTTG |
| 251 | CCATTCTAAA TCAAGCGGGT CTTCCACGTA TGAAAGGGAC |
| | AAGCCTGTTA |
| 301 | GATCTTTTTG CAAAACAAGG TCTTGTTCCT TCCGAGCTTC |
| | AGGAAAAAAT |
| 351 | CCGTTATCAA GAAGGCTTAT CAGAACAGAT GGCCTCTACG |
| | ATTAGAAAAA |
| 401 | TGGATGGCGT TGTCGATGCC TCAQTACAGA TTTCCTTCAC |
| | TACAGAAAAT |
| 451 | GAAGATAATC TTCCTTTAAC AGCCTCTGTG TATATTAAGC |
| | ATCGAGGGGT |
| 501 | TTTGGGCAAT CCGAACAGCA TTATGGTTTC CAAAATTAAG |
| | CGCCTTATTG |
| 551 | CAAGTGCTGT TCCAGGACTT GTGCCAGAGA ACGTCTCTGT |
| | AGTGAGCGAT |
| 601 | CGCGCAGCTT ATAGTGATAT TACAATTAAT GGTCCTTGGG |
| | GATTAACAGA |
| 651 | AGAAATCGAT TATGTTTCTG TTTGGGGTAT TATTCTTGCG |
| | AAGTCTTCGC |
| 701 | TCACCAAATT CCGTCTCATT TTTTATGTCT TGATTCTCAT |
| | TTTATTTGTT |
| 751 | ATTTCTTGTG CTCTCCTTTG GGTCATTTGG AAAACTCATA |
| | CTCTCATTAT |
| 801 | GACTATGGGA GGTACAAAAG GGTTCTTCAA CCCTACACCA |
| | TATACAAAGA |
| 851 | ATGCCTTGGA AGCCAAGAAA GCCGAGGGAG CAGCTGCTGA |
| | CAAAGAGAAA |
| 901 | AAAGAAGATG CAGATTCACA GGGGGAAAGC AAAAATGCGG |
| | AAACCAGTGA |
| 951 | TAAAGACTCT AGTGATAAAG ATGCTCCAGA AGGAAGCAAT |
| | GAAATTGAGG |
| 1001 | GTGCTTAG |

The PSORT algorithm predicts an inner membrane location (0.650).

The protein was expressed in E. coli and purified as a GST-fusion product, as shown in FIG. 55A. The recombinant protein was used to immunise mice, whose sera were used in a Western blot (FIG. 55B) and for FACS analysis (FIG. 55C). A his-tagged protein was also expressed.

These experiments show that cp7140 is a surface-exposed and immunoaccessible protein, and that it is a useful immunogen. These properties are not evident from the sequence alone.

### Example 56

The following C. pneumoniae protein (PID 4377306) was expressed <SEQ ID 111; cp7306>:

| | |
|---|---|
| 1 | MITKQLRSWL AVLVGSSLLALPLSGQAVGK KESRVSELPQ |
| | DVLLKEISGG |
| 51 | FSKVATKATP AVVYTESFPK SQAVTHPSPG RRGPYENPFD |
| | YFNDEPFNRP |
| 101 | FGLPSQREKP QSKFAVRGTG FLVSPDGYIV TNNHVVEDTG |
| | KIHVTLHDGQ |
| 151 | KYPATVIQLD PKTDLAVIKI KSQNLPYLSF GNSDHLKVGD |
| | WAIAIGNPFG |
| 201 | LQATVTVGVI SARGRNQLRI ADFEDFXQPD AAINPGNSGG |
| | PLLNIDGQVI |
| 251 | GVNTAIVSGS GGYIGIGFAI PSLMANRTID QLIRDGQVTR |
| | GFLGVTLQPI |
| 301 | DAELAACYKL EKVYGALVTD VVKGSPADRA GLKQEDVIIA |
| | YNGKEVDSLS |
| 351 | MFRNAVSLMN PDTRIVLKVV REGKVIEIPV TVSQAPKEDG |
| | MSALQRVGIR |
| 401 | VQNLTPETAK RLGIAPETXG ILIISVSPGS VAASSGIAPG |
| | QLILAVNRQK |
| 451 | VSSIEDLNRT LKDSNNENIL LMVSQGPVIR FIALKPEE* |

A predicted signal peptide is highlighted.

The cp7306 nucleotide sequence <SEQ ID 112> is:

| | |
|---|---|
| 1 | ATGATAACTA AGCAATTGCG TTCGTGGCTA GCTGTACTTG |
| | TTGGTTCAAG |
| 51 | TCTGCTAGCT CTTCCTTTAT CAGGGCAAQC TGTCGGGAAA |
| | AAAGAATCTC |
| 101 | GAGTTTCCGA GCTGCCTCAA GACGTTCTTC TTAAAGAGAT |
| | CTCGGGAGGG |
| 151 | TTTTCTAAGG TCGCTACCAA GGCGACTCCC GCTGTTGTGT |
| | ACATAGAAAG |
| 201 | TTTCCCAAAG AGCCAGGCTG TAACACATCC TTCTCCPGGA |
| | CGCCGTGGGC |
| 251 | CTTATGAAAA TCCTTTTGAT TATTTTAATG ATGAGTTTTT |
| | CAATCGTTTT |
| 301 | TTTGGTCTAC CTTCACAGAG GGAAAAACCT CAAAGTAAAG |
| | AGGCGGTTCG |
| 351 | AGGAAGAGGT TTCCTAGTAT CTCCAGATGG CTATATTGTG |
| | ACTAATAACC |
| 401 | ATGTTGTCGA AGATACAGGT AAGATTCACG TAACTCTTCA |
| | TGATGGGCAA |
| 451 | AAGTACCCAG CAACTGTAAT CGGACTCGAT CCTAAAACAG |
| | ACCTTGCAGT |
| 501 | CATTAAAATT AAATCCCAAA ACCTCCCGTA TCTTTCTTTT |
| | GGAAACTCCG |
| 551 | ACCACTTAAA AGTCGGAGAT TGGGCAATTG CAATTGGAAA |
| | TCCCTTCGGT |
| 601 | CTTCAAGCTA CGGTCACCGT AGGTGTCATC AGTGCTAAAG |
| | GAAGAAATCA |
| 651 | ACTCCACATT GCAGATTTTG AAGATTTTAT TCAGACAGAT |
| | GCTGCGATTA |
| 701 | ATCCAGGCAA CTCTGGAGGC CCTCTTCTAA ATATTGATGG |
| | ACAGGTCATC |
| 751 | GGTGTTAATA CTGCCATTGT CAGTGGTAGT GGTGGCTATA |
| | TTGGAATCGG |
| 801 | GTTTGCGATT CCTAGCCTTA TGGCAAATAG AATCATAGAT |
| | CAGCTGATTC |
| 851 | GTGATGGTCA AGTTACCCGA GGATTCTTAG GAGTGACTTT |
| | ACAACCTATA |
| 901 | GATGCGGAAC TCGCTGCTTG CTACAAACTC GAAAAGGTTT |
| | ATGGCGCTTT |
| 951 | AGTCACAGAT GTTGTTAAAG GATCTCCAGC AGATAAAGCA |
| | GGGCTAAAAC |
| 1001 | AAGAAGATGT GATCATTCCT TATAATGGGA AAGAAGTCGA |
| | TTCACTGAGT |
| 1051 | ATGTTCCGTA ATGCTGTTTC TTTAATGAAT CCAGATACAC |
| | GTATTGTTCT |
| 1101 | AAAGGTAGTT CGTGAAGGGA ACGTTATCGA AATACCCGTG |
| | ACAGTTTCTC |
| 1151 | AAGCTCCAAA AGAAGATGGA ATGTCGGCTT TACAGCGTGT |
| | GGGAATCCGT |
| 1201 | GTGCAAAACC TAACTCCTGA AACTGCTAAG AAGCTGGGAA |
| | TTGCTCCAGA |
| 1251 | GACTAAAGGC ATTTTGATTA TAAGTGTTGA ACCAGGQTCT |
| | GTAGCAGCTT |
| 1301 | CTTCAGGAAT TGCTCCTGGT CAGCTGATCC TTGCTGTGAA |
| | TAGACAAAAA |
| 1351 | GTATCTTCGA TTGAAGATCT GAATAGAACG TTAAAAGATT |
| | CTAACAATGA |
| 1401 | GAATATTCTT CTTATGGTTT CTCAAGGAGA TGTTATTCGC |
| | TTCATTGCCC |
| 1451 | TGAAACCTGA AGAATAA |

The PSORT algorithm predicts a periplasmic location (0.923).

The protein was expressed in E. coli and purified as a his-tag product (FIG. 56A) and as a GST-fusion product FIG. 56B). The recombinant proteins were used to immunise mice, whose sera were used in a Western blot (FIG. 56C) and for FACS (FIG. 56D) analyses.

The cp7306 protein was also identified in the 2D-PAGE experiment (Cpn0979) and showed good cross-reactivity with human sera, including sera from patients with pneumonitis.

These experiments show that cp7306 is a surface-exposed and immunoaccessible protein, and that it is a useful immunogen. These properties are not evident from the sequence alone.

### Example 57

The following C. pneumoniae protein (PID 4377132) was expressed <SEQ ID 113; cp7132>:

| | |
|---|---|
| 1 | MCNSIAMKKQ KRGFVLMELL MSFTLIALLL GTLGFWYRKI |
| | YTVQKQKERI |
| 51 | YNFYXEESRA YKQLRTLFSN SLSSSYEEPG SLFSLIFDRG |
| | VYRDPKLAGA |
| 101 | VRASLHHDTK DQRLELRICN IKDQSYFETQ RLLSHVTHVV |
| | LSFQRNPDPE |
| 151 | KLPETIALTI TREPKAYPPR TLTYQFAVGI* |

A predicted signal peptide is highlighted.

The cp7132 nucleotide sequence <SEQ ID 114> is:

| | |
|---|---|
| 1 | ATGTGTAACT CTATAGCTAT GAAAAAGCAA AAGCGTGGCT |
| | TTGTGCTTAT |
| 51 | GGAATTACTC ATGTCGTTCA CTCTAATTGC TTTGTTATTA |
| | GGGACTTTAG |
| 101 | GATTTTGGTA TCGGAAAATT TATACTGTAC AAAAGCAAAA |
| | AGAACGTATT |
| 151 | TATAACTTTT ATATCGAAGA GAGCCGAGCC TACAAGCAGC |
| | TCAGAACCCT |
| 201 | GTTTAGCATG TCCTTGTCTT GATCTTACGA GGAGCCTGGA |
| | TCATTATTTT |
| 251 | CTTTAATCTT TGATCGGGGG GTTTATCGAG ATCCTAAGCT |
| | GGCAGGTGCG |
| 301 | GTACGAGCTT CTCTCCATCA TGACACCAAG GATCAGAGAT |
| | TGGAACTTCG |
| 351 | TATTTGTAAT ATTAAGGATC AGTCTTACTT TGAAACACAG |
| | CGACTGCTCT |
| 401 | CCCACGTGAC CCATGTTGTA CTTTCCTTCC AGAGAAATCC |
| | TGATCCTGAA |
| 451 | AAACTTCCTG AAACAATTGC TTTAACTATA ACACGGGAAC |
| | CTAAAGCATA |
| 501 | TCCTCCAAGG ACGTTAACAT ACCAATTTGC GGTTGGGAAA |
| | TAA |

The PSORT algorithm predicts a periplasmic location (0.915).

The protein was expressed in E. coli and purified as a his-tag product (FIG. 57A) or as a GST-fusion. The recombinant proteins were used to immunise mice, whose sera were used in a Western blot (FIG. 57B) and FACS (FIG. 57C) analyses.

These experiments show that cp7132 is a surface-exposed and immunoaccessible protein, and that it is a useful immunogen. These properties are not evident from the sequence alone.

### Example 58

The following C. pneumoniae protein (PID 4376733) was expressed <SEQ ID 115; cp6733>:

| | |
|---|---|
| 1 | MKTSIPWVLV SSVLAFSCHL QSLANEELLS PDDSFNGNID |
| | SGTFTPKTSA |
| 51 | TTYSLTGDVF FYEPGKGTPL SDSCFKQTTD NLTFLGNGHS |
| | LTFGFIDAGT |
| 101 | HAGAAASTTA NKNLTFSGFS LLSFDSSPST TVVTGQGTLS |
| | SAGGVNLENI |
| 151 | RKLVVAGNPS TADGGAIKGA SFLLTGTSGD ALFSNNSSST |
| | KGGAIATTAG |
| 201 | ARIANNTGYV RFLSNIASTS GGAIDDEGTS ILSNNKFLYF |
| | EGNAAKTTGG |
| 251 | AICNTKASGS PELIISNNKT LIFASNVAET SGGAIHAXKL |
| | ALSSGGFTEF |
| 301 | LRNNVSSATP KGGAISIDAS GELSLSAETQ NITFVRNTLT |
| | TTGSTDTPKR |
| 351 | NAINIGSNGK FTELRAAKNH TIFFYDPITS EGTSSDVLKI |
| | NNGSAGALNP |
| 401 | YQGTILFSGE TLTADELKVA DNLKSSETQP VSLSGGKLLL |
| | QKGVTLESTS |
| 451 | FSQEAGSLIG MDSGTTLSTT AGSITITNLG INVDSLGLKQ |
| | PVSLTAKGAS |
| 501 | NKVIVSGKLN LIDIEGNIYE SHMFSHDQLF SLLKITVDAD |
| | VDTNVDISSL |
| 551 | IPVPAEDPNS EYGFQGQWNV NWTTDTATNT KEATATWTKT |
| | GFVPSPERKS |
| 601 | ALVCNTLWGV FTDIRSLQQL VEIGATGMEH KQGFWVSSMT |
| | NPLEKTGDEN |
| 651 | RKGFRHTSGG YVIGGSAHTP KDDLFTFAFC HLFARDKDCF |
| | IAHNNSRTYG |
| 701 | GTLFFKHSHT LQPQNYLRLG RAKFSESAIE KFPREIPLAL |
| | DVQVSFSHSD |
| 751 | NRMETHYTSL PESEGSWSNE CIAGGIGLDL PFVLSNPHPL |
| | FKTFIPQMKV |
| 801 | EMVYVSQNSF FESSSDGRGF SIGRLLNLSI PVGAKFVQGD |
| | IGDSYTYDLS |
| 851 | GPFVSDVYRN NPQSTATLVM SPDSWKIRGG NLSRQAPLLR |
| | CSNNYVYNSN |
| 901 | CELFGHYAME LRGSSRYNNV DVGTKLRF* |

A predicted signal peptide is highlighted.

The cp6733 nucleotide sequence <SEQ ID 116> is:

| | |
|---|---|
| 1 | ATGAAGACTT CGATTCCTTG GCTTTTAGTT TCCTCCGTGT |
| | TAGCTTTCTC |
| 51 | ATGTCACCTA CAGTCACTAG CTAACGAGGA ACTTTTATCA |
| | CCTGATGATA |
| 101 | GCTTTAATGG AAATATCGAT TCAGGAACGT TTACTCCAAA |
| | AACTTCAGCC |
| 151 | ACAACATATT CTCTAACAGG AGATGTCTTC TTTTACGAGC |
| | CTGGAAAAGG |
| 201 | CACTCCCTTA TCTGACAGTT GTTTTAAGCA AACCACGGAC |
| | AATCTTACCT |
| 251 | TCTTGGGGAA CGGTCATAGC TTAACGTTTG GCTTTATAGA |
| | TGCTGGCACT |
| 301 | CATGCAGGTG CTGCTGCATC TACAACAGCA AATAAGAATC |
| | TTACCTTCTC |
| 351 | AGGGTTTTCC TTACTGAGTT TTGATTCCTC TCCTAGCACA |
| | ACGGTTACTA |
| 401 | CAGGTCAGGG AACGCTTTCC TCAGCAGGAG GCGTAAATTT |
| | AGAAAATATT |
| 451 | CGTAAACTTG TAGTTGCTGG GAATTTTTCT ACTGCAGATG |
| | GTGGAGCTAT |
| 501 | CAAAGGAGCG TCTTTCCTTT TAACTGGCAC TTCTGGAGAT |
| | GCTCTTTTTA |
| 551 | GTAACAACTC TTCATCAACA AAGGGAGGAG CAATTGCTAC |
| | TACAGCAGGC |
| 601 | GCTCGCATAG CAAATAACAC AGGTTATGTT AGATTCCGTT |
| | CTAACATAGC |
| 651 | GTCTACGTCA GGAGGCGCTA TCGATGATGA AGGCACGTCG |
| | ATACTATCGA |
| 701 | ACAACAAATT TCTATATTTT GAAGGGAATG CAGCGAAAAC |
| | TACTGGCGGT |
| 751 | GCGATCTGCA ACACCAAGGC GAGTGGATCT CCTGAACTGA |
| | TAATCTCTAA |
| 801 | CAATAAGACT CTGATCTTTG CTTCAAACGT AGCAGAAACA |
| | AGCGGTGGCG |
| 851 | CCATCCATGC TAAAAAGCTA GCCCTTTCCT CTGQAGGCTT |
| | TACAGAGTTT |
| 901 | CTACGAAATA ATGTCTCATC AGCAACTCCT AAGGGGGGTG |
| | CTATCAGCAT |
| 951 | CGATGCCTCA GGAGAGCTCA GTCTTTCTGC AGAGACAGGA |
| | AACATTACCT |
| 1001 | TTGTAAGAAA TACCCTTACA ACAACCGGAA GTACCGATAC |
| | TCCTAAACGT |
| 1051 | AATGCGATCA ACATAGGAAG TAACGGGAAA TTCACGGAAT |
| | TACGGGCTGC |
| 1101 | TAAAAATCAT ACAATTTTCT TCTATGATCC CATCACTTCA |
| | GAAGGAACCT |
| 1151 | CATCAGACGT ATTGAAGATA AATAACGGCT CTGCGGGAGC |
| | TCTCAATCCA |
| 1201 | TATCAAGGAA CGATTCTATT TTCTGGAGAA ACCCTAACAG |
| | CAGATGAACT |
| 1251 | TAAAGTTGCT GACAATTTAA AATCTTCATT CACGCAGCCA |
| | GTCTCCCTAT |
| 1301 | CCGGAGGAAA GTTATTGCTA CAAAAGGGAG TCACTTTAOA |
| | GAGCACGAGC |
| 1351 | TTCTCTCAAG AGGCCGGTTC TCTCCTCGGC ATGGATTCAG |
| | GAACGACATT |
| 1401 | ATCAACTACA GCTGGGAGTA TTACAATCAC GAACCTAGGA |
| | ATCAATGTTG |
| 1451 | ACTCCTTAGG TCTTAAGCAG CCCGTCAGCC TAACAGCAAA |
| | AGGTGCTTCA |
| 1501 | AATAAAGTGA TCGTATCTGG GAAGCTCAAC CTGATTGATA |
| | ATGAAGGCAA |
| 1551 | CATTTATGAA AGTCATATGT TCAGCCATGA CCAGCTCTTC |
| | TCTCTATTAA |
| 1601 | AAATCACGGT TGATGCTGAT GTTGATACTA ACGTTGACAT |
| | CAGCAGCCTT |
| 1651 | ATCCCTGTTC CTGCTGAGGA TCCTAATTCA GAATACGGAT |
| | TCCAAGGACA |
| 1701 | ATGGAATGTT AATTGGACTA CGGATACAGC TACAAATACA |
| | AAAGAGGCCA |
| 1751 | CGGCAACTTG GACCAAAACA GGATTTGTTC CCAGCCCCGA |
| | AAGAAAATCT |
| 1801 | GCGTTAGTAT GCAATACCCT ATGGGGAGTC TTTACTGACA |
| | TTCGCTCTCT |
| 1851 | GCAACAGCTT GTAGAGATCG GCGCAACTGG TATGGAACAC |
| | AAACAAGGTT |
| 1901 | TCTGGGTTTC CTCCATGACG AACTTCCTGC ATAAGACTGG |
| | AGATGAAAAT |
| 1951 | CGCAAAGGCT TCCGTCATAC CTCTGGAGGC TACGTCATCG |
| | GTGGAAGTGC |
| 2001 | TCACACTCCT AAAGACGACC TATTTACCTT TGCGTTCTGC |
| | CATCTCTTTG |
| 2051 | CTAGAGACAA AGATTGTTTT ATCGCTCACA ACAACTCTAG |
| | AACCTACGGT |
| 2101 | GGAACTTTAT TCTTCATGCA CTCTCATACC CTACAACCCC |
| | AAAACTATTT |
| 2151 | GAGATTAGGA AGAGCAAAGT TTTCTGAATC AGCTATAGAA |
| | AAATTCCCTA |
| 2201 | GGGAAATTCC CCTAGCCTTG GATGTCCAAG TTTCGTTCAG |
| | CCATTCAGAC |
| 2251 | AACCGTATGG AAACGCACTA TACCTCATTG CCAGAATCCG |
| | AAGGTTCTTG |
| 2301 | GAGCAACGAG TGTATAGCTG GTGGTATCGG CCTAGACCTT |
| | CCTTTTGTTC |
| 2351 | TTTCCAACCC ACATCCTCTT TTCAAGACCT TCATTCCACA |
| | GATGAAAGTC |
| 2401 | GAAATGGTTT ATGTATCACA AAATAGCTTC TTCGAAAGCT |
| | CTAGTGATGG |
| 2451 | CCGTGGTTTT AGTATTGGAA GGCTGCTTAA CCTCTCGATT |
| | CCTGTGGGTG |
| 2501 | CGAAATTCGT GCAGGGGGAT ATCGGAGATT CCTACACCTA |
| | TGATCTCTCA |
| 2551 | GGATTCTTTG TTTCCGATGT CTATCGTAAC AATCCCCAAT |
| | CTACAGCGAC |
| 2601 | TCTTGTGATG AGCCCAGACT CTTGGAAAAT TCGCGGTGGC |
| | AATCTTTCAA |
| 2651 | GACAGGCATT TTTACTGAGG GGTAGCAACA ACTACGTCTA |
| | CAACTCCAAT |
| 2701 | TGTGAGCTCT TCGGACATTA CGCTATGGAA CTCCGTGGAT |
| | CTTCAAGGAA |
| 2751 | CTACAATGTA GATGTTGGTA CCAAACTCCG ATTCTAG |

The PSORT algorithm predicts an outer membrane location (0.924).

The protein was expressed in E. coli and purified as a his-tag product, as shown in FIG. 58A. The recombinant protein was used to immunise mice, whose sera were used in a Western blot (FIG. 58B) and for FACS (FIG. 58C) analyses. A GST-fusion protein was also expressed.

The cp6733 protein was also identified in the 2D-PAGE experiment (Cpn0451).

These experiments show that cp6733 is a surface-exposed and immunoaccessible protein, and that it is a useful immunogen. These properties are not evident from the sequence alone.

### Example 59

The following C. pneumoniae protein (PID 4376814) was expressed <SEQ ID 117; cp6814>:

| | |
|---|---|
| 1 | MHDALLSILA IQELDIKMIR LMRVKKEHQK ELAKVQSLKS |
| | DIRRVQEKE |
| 51 | LEMENLKTQI RDGENRIQRI SEQINKLENQ QAAVKKMDEF |
| | NALTQEMTTA |
| 101 | NKERRSLEHQ LSDLMDKQAG GEDLIVSLKE SLASTENSSS |
| | VIEKEIFESI |
| 151 | KKINEEGKAL LEQRTELKHA TNPELLSIYE RLLNNKKDRV |
| | VVPIENRVCS |
| 201 | GCHIVLTPQH ENLVRKKDRL IECEECSRIL YWQESQVNAQ |
| | ENSTAXEERR |
| 251 | RAAV* |

The cp6814 nucleotide sequence <SEQ ID 118> is:

| | |
|---|---|
| 1 | ATGCATGACG CACTTCTAAG CATTTTGGCT ATTCAAGAGC |
| | TTGATATTAA |
| 51 | AATGATTCGC CTTATGCGCG TAAAGAAAGA ACATCAGAAA |
| | GAATTGGCTA |
| 101 | AAGTCCAATC TTTAAAAAGT GATATTCGTA GAAAAGTTCA |
| | GGAAAAAGAA |
| 151 | CTCGAAATGG AGAATTTGAA AACTCAAATT CGAGATGGAG |
| | AGAATCGCAT |
| 201 | CCAAGAGATT TCTGAACAAA TCAATAAATT AGAAAATCAG |
| | CAAGCTGCTG |
| 251 | TAAAAAAAAT GGATGAGTTT AACGCTCTCA CCCAAGAAAT |
| | GACTACAGCA |
| 301 | AACAAAGAAC GTCGCTCTTT AGAGCACCAG CTTAGCGATC |
| | TCATGGATAA |
| 351 | GCAAGCTTGA GGCGAAGACC TTATTGTCTC TCTAAAAGAA |
| | AGCTTAGCTT |
| 401 | CTACAGAAAA TAGTAGCAGT GTCATTGAAA AAGAAATTTT |
| | TGAAAGCATC |
| 451 | AAAAAGATTA ATGAAGAAGG CAAAGCTTTG CTTGAACAAC |
| | GGACAGAGTT |
| 501 | AAAGCATGCG ACGAATCCCG AACTACTCAG CATCTATGAG |
| | CGTCTATTAA |
| 551 | ACAATAAAAA AGATCGCGTT GTTGTTCCTA TTGAAAATCG |
| | TGTCTGCAGT |
| 601 | GGTTGTCATA TTGTTCTAAC TCCTCAACAC GAAAATCTTG |
| | TAAGAAAGAA |
| 651 | AGACCGACTC ATTTTTTGCG AACATTGCTC TCGAATTCTC |
| | TATTGGCAAG |
| 701 | AATCCCAAGT CAATGCTCAG GAAAATTCCA CAGCAAAACG |
| | TCGTCGTCGT |
| 751 | CGCGCAGCTG TATAA |

The PSORT algorithm predicts an inner membrane location (0.070).

The protein was expressed in E. coli and purified as a GST-fusion (FIG. 59A) or his-tagged product. The recombinant proteins were used to immunise rice, whose sera were used in Western blot (FIG. 59B) and FACS (FIG. 59C) analyses.

These experiments show that cp6814 is a surface-exposed and immunoaccessible protein, and that it is a useful immunogen. These properties are not evident from the sequence alone.

### Example 60

The following C. pneumoniae protein (PID 4376830) was expressed <SEQ ID 119; cp6830>:

| | |
|---|---|
| 1 | MKWLPATAVF AAVLPALTAF G DPASVEIST SHTGSGDPTS |
| | DAALTGFTQS |
| 51 | STETDGTTYT IVGDITPSTF TNIPVPVVTP DANDSSSNSS |
| | KGGSSSSGAT |
| 101 | SLIRSSNLHS DFDFTKDSVL DLYHLFFPSA SNTLNPALLS |
| | SSSSGGSSSS |
| 151 | SSSSSSGSAS AVVAADPKGG AAFYSNEANG TLTFTTDSGN |
| | PGSLTLQNLK |
| 201 | MTGDGAAIYS KGPLVFTGLK NLTFTGNESQ KSGGAAYTEG |
| | ALTTQAIVEA |
| 251 | VTFTGNTSAG QGGAIYVKEA TLFNALDSLK FEKNTSGQAG |
| | GGIYTESTLT |
| 301 | ISNITKSIEF ISNXASVPAP APBPTSPAPS SLINSTTIDT |
| | STLQTRAASA |
| 351 | TPAVAPVAAV TPTPISTQET AGNGGAIYAK QGTSISTSKD |
| | LTFKSNSASV |
| 401 | DATLTVDBST IGESGGAIFA ADSIQIQQCT GTTLFSGMTA |
| | NXSGGGIYAV |
| 451 | GQVTLEDIAN LKMTNNTCKG EGGAIYTKKA LTINNGAILT |
| | TFSGNTSTDN |
| 501 | GGAIFAVGGI TLSDLVEVRF SKNKTGNYSA PITKAASNTA |
| | PVVSSSTTAA |
| 551 | SPAVPAAAAA PVTNAAKGGA LYSTEGLTVS GITSILSFEN |
| | NECQNQGGGA |
| 601 | YVTKTFQCSD SHRLQFTSNK AADEGGGLYC GDDVTLTNLT |
| | GKTLFQEWSS |
| 651 | EKHGGGLSLA SGKSLTMTSL E8FCLNANTA KENGGGANVP |
| | ENIVLTFTYT |
| 701 | PTPNEPAPVQ QPVYGEALVT GNTATKSGGG IYTKNAAFSN |
| | LSSVTFDQNT |
| 751 | SSENGGALLT QKAADKTDCS FTYITNVNIT NNTATGNGGG |
| | IAGGKAHFDR |
| 801 | IDNLTVQSNQ AKKGGGVYLE DALILEKVIT GSVSQNTATE |
| | SGGGIYAKDI |
| 851 | QLQALPGSFT ITDNKVBTSL TTSTNLYGGG IYSSGAVTLT |
| | NISGTFGITG |
| 901 | NSVINTATSQ DADIQGGGIY ATTSLSINQC NTPILFSNNS |
| | AATKKTSTTK |
| 951 | QIAGGAIESA AVTIENNSQP IIFLNNSAKS EATTAATAGN |
| | KDSCGGAIAA |
| 1001 | NSVTLTNNPE ITFKGNYAET GGAIGCIDLT NGSPPRKVSI |
| | ADNGSVLFQD |
| 1051 | NSALNRGGAI YGETIDISRT GATFIGNSSR HDGSAICCST |
| | ALTLAPNSQL |
| 1101 | IFENNKVTET TATTKABINN LGAAIYGNNE TSDVTISLSA |
| | ENGSIFFKNN |
| 1151 | LCTATNKYCS IAGNVKFTAI EASAGRAISE YDAVNVSTKE |
| | TNAQELKLNE |
| 1201 | KATSTGTILF SGELHENKSY IPQKVTFAHG NLILGKNAEL |
| | SVVSFTQSPG |
| 1251 | TTITMGPGSV LSNHSKEAGG IAINNVIIDF SEIVPTKDNA |
| | TVAPPTLKLV |
| 1301 | SRTNADSKDK IDITGTVTLL DPNGNLYQNS YLGEDRDITL |
| | FNIDNSASGA |
| 1351 | VTATNVTLQG NLGAKKGYLG TWNLDPNSSG SKIILKWTFI |
| | KYLRWPYIPR |
| 1401 | DNHFYINSIW GAQNSLVTVK QGILGNMLNN ASPEDPAFNN |
| | FWASAIGSFL |
| 1451 | RKEVSRNSDS FTYHGRGYTA AVDAKPRQEF ILGAAFSQVF |
| | GHAESEYHLD |
| 1501 | NYKHKGSGHS TQASLYAGNI FYFPAIRSRP ILPQGVATYG |
| | YNQHDTTTYY |
| 1551 | PSIEEKNMAN WDSIAWLFDL RFSVDLKEPQ PHSTARLTFY |
| | TEAEYTRIRQ |
| 1601 | EKFTELDYDP RSPSACSYGN LAIPTGFSVD GALAWREIIL |
| | YNKVSAAYLP |
| 1651 | VILRNNPKAT YEVLSTKEKG NVVNVLPTRN AARAEVSSQI |
| | YLGSYWTLYG |
| 1701 | TYTIDASMNT LVQMANGGIR FVF* |

A predicted signal peptide is highlighted.

The cp6830 nucleotide sequence <SEQ ID 120> is:

| | |
|---|---|
| 1 | ATGAAGTGGC TACCAGCTAC AGCTGTTTTT GCTGCCGTAC |
| | TCCCCGCACT |
| 51 | AACAGCCTTC GGAGATCCCG CGTCTGTTGA AATAAGTACC |
| | AGCCATACAG |
| 101 | GATCCGGGGA TCCTACAAGC GACGCTGCCT TAACAGGATT |
| | TACACAAAGT |
| 151 | TCCACAGAAA CTGACGGTAC TACCTATACC ATTGTCGGTG |
| | ATATCACCTT |
| 201 | CTCTACTTTT ACGAATATTC CTGTTCCCGT AGTAACTCCA |
| | GACGCCAACG |
| 251 | ATAGTTCCAG CAATAGCTCT AAAGGAGGAA GTAGCAGTAG |
| | TGGAGCTACA |
| 301 | TCTCTAATCC GATCCTCAAA CCTACACTCC GATTTTGATT |
| | TTACAAAAGA |
| 351 | TAGCGTGTTA GACCTCTATC ACCTTTTCTT TCCTTCAGCT |
| | TCAAATACTC |
| 401 | TCAATCCTGC ACTCCTTTCT TCCAGTAGCA GCGGTGGATC |
| | CTCGAGCAGC |
| 451 | AGTAGCTCCT CATCATCTGG AAGTGCATCT GCTGTTGTTG |
| | CTGCGGACCC |
| 501 | AAAAGGAGGC GCTGCCTTTT ATAGTAACGA GGCTAACGGA |
| | ACTTTAACCT |
| 551 | TCACTACAGA CTCTGGAAAT CCCGGCTCCC TGACTCTTCA |
| | GAATCTTAAA |
| 601 | ATGACCGGAG ATGGAGCCGC CATCTACTCG AAGGGTCCTC |
| | TAGTATTTAC |
| 651 | TGGTTTAAAA AATCTAACCT TTACAGGAAA TGAATCTCAG |
| | AAATCTGGAG |
| 701 | GTGCTGCCTA TACTGAAGGC GCACTCACAA CACAAGCAAT |
| | CGTTGAAGCC |
| 751 | GTAACTTTTA CTGGCAACAC CTCGGCAGGG CAAGGAGGCG |
| | CTATCTATGT |
| 801 | TAAAGAAGCT ACCCTATTCA ATGCTCTAGA CAGCCTCAAA |
| | TTTGAAAAAA |
| 851 | ACACTTCTGG GCAAGCTGGT GGTGGAATCT ATACAGAGTC |
| | TACGCTCACA |
| 901 | ATCTCGAACA TCACAAAATC TATTGAATTT ATCTCTAATA |
| | AAGCTTCTGT |
| 951 | CCCTGCCCCC GCTCCTGAGC CCACCTCTCC GGCTCCAAGT |
| | AGCTTAATAA |
| 1001 | ATTCTACAAC GATCGATACC TCGACTCTCC AAACCCGAGC |
| | AGCATCCGCA |
| 1051 | ACTCCAGCAG TGGCTCCTGT TGCTGCCGTA ACTCCAACAC |
| | CAATCTCTAC |
| 1101 | TCAAGAGACC GCAGGAAATG GAGGCGCTAT CTATGCTAAA |
| | CAAGGTATTT |
| 1151 | CGATATCCAC GTTTAAAGAT CTGACCTTCA AGTCTAACTC |
| | TGCATCGGTA |
| 1201 | GATGCCACCC TTACTGTCGA TTCTAGCACT ATTGGAGAAT |
| | CTGGAGGTGC |
| 1251 | TATCTTTGCA GCAGACTCTA TACAAATCCA ACAGTGCACG |
| | GGAACCACCT |
| 1301 | TATTCAGTGG CAATACTGCC AATAAGTCTG GTGGGGGTAT |
| | TTACGCTGTA |
| 1351 | GGACAAGTCA CCCTAGAAGA TATAGCGAAT CTGAAGATGA |
| | CCAACAACAC |
| 1401 | CTGTAAAGGT GAAGGTGGAG CCATCTACAC TAAAAAGGCT |
| | TTAACTATCA |
| 1451 | ACAACGGTGC CATTCTCACT ACATTTTCTG GAAATACATC |
| | GACAGATAAT |
| 1501 | GGTGGGGCTA TTTTTGCTGT AGGTGGCATC ACTCTCTCTG |
| | ATCTTGTAGA |
| 1551 | AGTCCGCTTT AGTAAAAATA AGACCGGAAA TTATTCCGCT |
| | CCTATTACCA |
| 1601 | AAGCGGCTAG CAACACAGCT CCTGTAGTTT CTAGCTCTAC |
| | AACTGCTGCA |
| 1651 | TCTCCTGCGG TCCCTGCTGC CGCTGCAGCA CCTGTTACAA |
| | ACGCAGCAAA |
| 1701 | AGGAGGGGCT TTATATAGTA CAGAAGGACT GACTGTATCT |
| | GGAATCACAT |
| 1751 | CGATATTGTC GTTTGAAAAC AACGAATGCC AGAATCAAGG |
| | AGGTGGGGCT |
| 1801 | TACGTTACTA AAACCTTCCA GTGTTCCGAT TCTCATCGCC |
| | TCCAGTTTAC |
| 1851 | TAGTAATAAA GCAGCAGATG AAGGCGGGGG CCTGTATTGT |
| | GGTGACGATG |
| 1901 | TCACGCTAAC GAACCTGACA GGGAAAACAC TATTTCAAGA |
| | GAATAGCAGT |
| 1951 | GAGAAACATG GAGGTGGGCT CTCTCTCGCC TCAGGAAAAT |
| | CTCTGACTAT |
| 2001 | GACATCGTTA GAGAGCTTCT GCTTAAATGC AAATACAGCA |
| | AAGGAAAACG |
| 2051 | GAGGCGGTGC GAATGTCCCT GAAAATATTG TACTCACCTT |
| | CACCTATACT |
| 2101 | CCCACTCCAA ATGAACCTGC GCCTGTGCAG CAGCCCGTGT |
| | ATGGAGAAGC |
| 2151 | TCTTGTTACT GGAAATACAG CCACAAAAAG TGGTGGGGGC |
| | ATTTACACGA |
| 2201 | AAAATGCGGC CTTCTCAAAT TTATCTTCTG TAACTTTTGA |
| | TCAAAATACC |
| 2251 | TCTTCAGAAA ATGGTGGTGC CTTACTTACC CAAAAAGCTC |
| | CAGATAAAAC |
| 2301 | GGACTGTTCT TTCACCTATA TTACAAATGT CAATATCACC |
| | AACAATACAG |
| 2351 | CTACAGGAAA TGGTGGGGGC ATTGCTGGGG GAAAAGCACA |
| | TTTCGATCGC |
| 2401 | ATTGATAATC TTACAGTCCA AAGCAACCAA GCAAAGAAAG |
| | GTGGTGGGGT |
| 2451 | TTATCTTGAA GATGCCCTCA TCCTGGAAAA GGTTATTACA |
| | GGTTCTGTCT |
| 2501 | CACAAAATAC AGCThCAGAA AGTGGTGGGG GTATCTACGC |
| | TAAGGATATT |
| 2551 | CAACTACAAG CTCTACCTGG AAGCTTCACA ATTACCGATA |
| | ATAAAGTCGA |
| 2601 | AACTAGTCTT ACTACTAGCA CTAATTTATA TGGTGGGGGC |
| | ATCTATTCCA |
| 2651 | GTGGAGCTGT CACGCPAACC AATATATCTG GAACCTTTGG |
| | CATTACAGGA |
| 2701 | AACTCTGTTA TCAATACAGC GACATCCCAG GATGCAGATA |
| | TACAAGGTGG |
| 2751 | GGGCATTTAT GCAACCACGT CTCTCTCAAT AAATCAATGT |
| | AATACACCCA |
| 2801 | TTCTATTTAG CAACAACTCT GCTGCCACTA AAAAAACATC |
| | AACAACAAAG |
| 2851 | CAAATTGCTG GTGGGGCTAT CTTCTCCGCT GCAGTAACTA |
| | TCGAGAATAA |
| 2901 | CTCTCAGCCC ATTATTTTCT TAAATAATTC CGCAAAGTCG |
| | GAAGCAACTA |
| 2951 | CAGCAGCAAC TGCAGGAAAT AAAGATAGCT GTGGAGGAGC |
| | CATTGCAGCT |
| 3001 | AACTCTGTTA CTTTAACAAA TAACCCTGAA ATAACCTTTA |
| | AAGGAAATTA |
| 3051 | TGCAGAAACT GOAGGAGOGA TTGGCTGTAT TGATCTTACT |
| | AATGGCTCAC |
| 3101 | CTCCCCGTAA AGTCTCTATT GCAGACAACG GTTCTGTCCT |
| | TTTTCAAGAC |
| 3151 | AACTCTGCGT TAAATCGCGG AGGCGCTATC TATGGAGAGA |
| | CTATCGATAT |
| 3201 | CTCCAGGACA GGTGCGACTT TCATCGGTAA CTCTTCAAAA |
| | CATGATGGAA |
| 3251 | GTGCAATTTG CTGTTCAACA GCCCTAACTC TTGCGCCAAA |
| | CTCCCAACTT |
| 3301 | ATCTTTGAAA ACAATAAGGT TACGGAAACC ACAGCCACTA |
| | CAAAAGCTTC |
| 3351 | CATAAATAAT TTAGGAGCTG CAATTTATGG AAATAATGAG |
| | ACTAGTGACG |
| 3401 | TCACTATCTC TTTATCAGCT GAGAATGGAA GTATTTTCTT |
| | TAAAAACAAT |
| 3451 | CTATGCACAG CAACAAACAA ATACTGCAGT ATTGCTGGAA |
| | ACGTAAAATT |
| 3501 | TACAGCAATA GAAGCTTCAG CAGGGAAAGC TATATCTTTC |
| | TATGATGCAG |
| 3551 | TTAACGTTTC CACCAAAGAA ACAAATGCTC AAGAGCTAAA |
| | ATTAAATGAA |
| 3601 | AAAGCGACAA GTACAGGAAC GATTCTATTT TCTGGGGAAC |
| | TTCACGAAAA |
| 3651 | TAAATCCTAT ATTCCACAGA AAGTCACTTT CGCACATGGG |
| | AATCTCATTC |
| 3701 | TAGGTAAAAA TGCAGAACTT AGCGTAGTTT CCTTTACCCA |
| | ATCTCCAGGC |
| 3751 | ACCACAATCA CTATGGGCCC AGGATCGCTT CTTTCCAACC |
| | ATAGCAAAGA |
| 3801 | AGOAGGAGGA ATCGCTATAA ACAATGTCAT CATTGATTTT |
| | AGTGAAATCG |
| 3851 | TTCCTACTAA AGATAATGCA ACAGTAGCTC CACCCACTCT |
| | TAAATTAGTA |
| 3901 | TCGAGAACTA ATGCAGATAG TAAAGATAAG ATTGATATTA |
| | CAGGAACTGT |
| 3951 | GACTCTTCTA GATCCTAATG GCAACTTATA TCAAAATTCT |
| | TATCTTGGTG |
| 4001 | AAGACCGCGA TATCACTCTT TTCAATATAG ACAATTCTGC |
| | AAGTGGGGCA |
| 4051 | GTTACAGCCA CGAATGTCAC CCTTCAAGGG AATTTAGGAG |
| | CTAAAAAAGG |
| 4101 | ATATTTAGGA ACCTGGAATT TGGATCCAAA TTCCTCGGGT |
| | TCAAAAATTA |
| 4151 | TTCTAAAATG GACCTTTGAC AAATACCTGC GCTGGCCCTA |
| | CATCCCTAGA |
| 4201 | GACAACCACT TCTACATCAA CTCTATTTGG GGAGCACAAA |
| | ACTCTTTAGT |
| 4251 | GACTGTGAAA CAAGGGATCT TAGGGAACAT GTTGAACAAT |
| | GCAAGGTTTG |
| 4301 | AAGATCCTGC TTTCAACAAC TTCTGGGCTT CGGCTATAGG |
| | ATCTTTCCTT |
| 4351 | AGGAAAGAAG TATCTCGAAA TTCTGACTCA TTCACCTATC |
| | ATGGCAGAGG |
| 4401 | CTATACCGCT GCTGTGGATG CCAAACCTCG CCAAGAATTT |
| | ATTTTAGGAG |
| 4451 | CTGCCTTCAG TCAGGTTTTT GGTCACGCCG AGTCTGAATA |
| | TCACCTTGAC |
| 4501 | AACTATAAGC ATAAAGGCTC AGGTCACTCT ACACAAGCAT |
| | CTCTTTATGC |
| 4551 | TGGCAATATC TTCTATTTTC CTGCGATACG GTCTCGGCCT |
| | ATTCTATTCC |
| 4601 | AAGGTGTGGC GACCTATGGT TATATGCAAC ATGACACCAC |
| | AACCTACTAT |
| 4651 | CCTTCTATTG AAGAAAAAAA TATGGCAAAC TGGGATAGCA |
| | TTGCTTGGTT |
| 4701 | ATTTGATCTG CGTTTCAGTG TGGATCTTAA AGAACCTCAA |
| | CCTCACTCTA |
| 4751 | CAGCAAGGCT TACCTTCTAT ACAGAAGCTG AGTATACCAG |
| | AATTCGCCAG |
| 4801 | GAGAAATTCA CAGAGCTAGA CTATGATCCT AGATCTTTCT |
| | CTGCATGCTC |
| 4851 | TTATGGAAAC TTAGCAATTC CTACTGGATT CTCTGTAGAC |
| | GGAGCATTAG |
| 4901 | CTTGGCGTGA GATTATTCTA TATAATAAAG TATCAGCTGC |
| | GTACCTCCCT |
| 4951 | GTGATTCTCA GGAATAATCC AAAAGCGACC TATGAAGTTC |
| | TCTCTACAAA |
| 5001 | AGAAAAGGGC AACGTAGTCA ACGTTCTCCC TACAAGAAAC |
| | GCAGCTCGTG |
| 5051 | CAGAGGTGAG CTCTCAAATT TATCTTGGAA GTTACTGGAC |
| ACTCTACGGC | |
| 5101 | ACGTATACTA TTGATGCTTC AATGAATACT TTAGTGCAAA |
| TGGCCAACGG | |
| 5151 | AGGGATCCGG TTTGTATTCT AG |

The PSORT algorithm predicts an outer membrane location (0.926).

The protein was expressed in E. coli and purified as a GST-fusion (FIG. 60A) or his-tagged product. The recombinant proteins were used to immunise mice, whose sera were used in Western blot (FIG. 60B) and FACS (FIG. 60C) analyses.

The cp6830 protein was also identified in the 2D-PAGE experiment (Cpn0540) and showed good cross-reactivity with human sera, including sera from patients with pneumonitis.

These experiments show that cp6830 is a surface-exposed and immunoaccessible protein, and that it is a useful immunogen. These properties are not evident from the sequence alone.

### Example 61

The following C. pneumoniae protein (PID 4376854) was expressed <SEQ ID 121; cp6854>:

| | |
|---|---|
| 1 | MSIAIAREQY AAILDMHPKP SIAMFSSEQA RTSWEKRQAH |
| | PYLYRLLEII |
| 51 | WGVVKFLLGL IFFIPLGLFW VLQKICQNFI LLGAGGWIFR |
| | PICRDSNLLR |
| 101 | QAYAARLFSA SEQDHVSSVR RVCLQYDEVF IDGLELRLPN |
| | AKPDRWMLIS |
| 151 | NQNSDCLEYR TVLQGEKDWI FRIAEESQSN ILIFNYPGVN |
| | KSQGNITRNN |
| 201 | VVKSYQACVR YLRDEPAGPQ ARQIVAYGYS LGASVQAEAL |
| | SKEIADGSDS |
| 251 | VRWFVVKDRG ARSTGAVAKQ FIGSLGVWLA NLTHWNINSE |
| | KRSKDLHCPE |
| 301 | LFIYQKDSQG NLIGDGLFKK ETCPAAPPLD PKNLEECSGK |
| | KIPVAQTGLR |
| 351 | HDHILSDDVI KEVAGHIQRH FDN* |

The cp6854 nucleotide sequence <SEQ ID 122> is:

| | |
|---|---|
| 1 | ATGTCAATAG CTATTGCAAG GGAACAATAC GCAGCTATAT |
| | TGGATATGCA |
| 51 | TCCTAAACCT TCGATCGCCA TGTTTTCTTC GGAGCAGGCG |
| | AGAACTTCTT |
| 101 | GGGAGAAACG ACAGGCTCAT CCTTACCTTT ATCGTCTTCT |
| | TGAGATCATA |
| 151 | TGGGGTGTTG TGAAATTTCT TCTCGGCTTA ATCTTCTTTA |
| | TTCCCTTGGG |
| 201 | TCTTTTCTGG GTCCTTCAGA AGATATGTCA GAATTTTATT |
| | CTTCTTGGTG |
| 251 | CAGGAGGGTG GATTTTTAGA CCCATATGCA GGGACTCTAA |
| | TTTATTGCGA |
| 301 | CAAGCTTACG CCGCGCGTCT TTTCTCCGCT TCATTCCAAG |
| | ATCATGTCTC |
| 351 | CTCTGTGCGA AQGGTTTGCT TACAGTATGA CGAGGTCTTT |
| | ATTGACGGAT |
| 401 | TGGAGTTACG TCTTCCCAAT GCTAAGCCAG ATCGATGGAT |
| | GTTAATCTCC |
| 451 | AATGGAAACT CCGATTGCTT AGAGTATAGG ACAGTGCTGC |
| | AAGGGGAAAA |
| 501 | GGACTGGATA TTCCGTATTG CTGAAGAGTC TCAATCCAAC |
| | ATTTTAATCT |
| 551 | TCAATTACCC AGGAGTCATG AAGAGCCAAG GGAATATAAC |
| | AAGAAACAAT |
| 601 | GTAGTCAAAT CTTATCAAGC ATGCQTACGC TATCTTAGAG |
| | ATGAACCCGC |
| 651 | AGGACCTCAG GCGCGTCAAA TCGTTGCTTA TGGCTATTCT |
| | TTAGGAGCTA |
| 701 | GTGTTCAAGC CGAAGCATTA AGTAAAGAGA TCGCAGACGG |
| | AAGTGATAGC |
| 751 | GTCOGTTGGT TTGTCGTTAA AGATCGAGGA GCTCGCTCTA |
| | CAGGAGCCGT |
| 801 | TGCTAAACAG TTTATTGGAA GTCTAGGAGT TTGGCTGGCG |
| | AATCTTACCC |
| 851 | ATTGGAATAT TAATTCTGAA AAGAGAAGCA AGGACTTGCA |
| | TTGCCCAGAA |
| 901 | CTCTTTATTT ATGGCAAGGA TTCCCAAGGT AATCTTATCG |
| | GGGATGGATT |
| 951 | GTTCAAAAAA GAGACGTGCT TCGCAGCACC ATTTTTAGAT |
| | CCTAAAAACT |
| 1001 | TGGAAGAGTG TTCAGGGAAG AAAATCCCTG TAGCTCAGAC |
| | CGGTCTAAGA |
| 1051 | CACGATCATA TCCTTTCCGA TGATGTGATT AAAGAAGTTG |
| | CAGGTCATAT |
| 1101 | TCAAAGACAT TTCGATAATT A |

The PSORT algorithm predicts an inner membrane location (0.461).

The protein was expressed in E. coli and purified as a GST-fusion product, as shown in FIG. 61A. The recombinant protein was used to immunise mice, whose sera were used in Western blot (FIG. 61B) and FACS (FIG. 61C) analyses. A his-tagged protein was also expressed.

These experiments show that cp6854 is a surface-exposed and immunoaccessible protein, and that it is a useful immunogen. These properties are not evident from the sequence alone.

### Example 62

The following C. pneumoniae protein (PID 4377101) was expressed <SEQ ID 123; cp7101>:

| | |
|---|---|
| 1 | MYSCYSKGIS HNYLLHPMSR LDIFVFDSLI ANQDQNLLEE |
| | EFCSEDTVLF |
| 51 | KAYRTTALQS PLAAKNLNIA RKVANYILAD NGEIDTVKLV |
| | EAIHHLSQCT |
| 101 | YPLGPHRHNE AQDREHLLKM LKALKENPKL KESIKTLFVP |
| | SYSTIQNLIR |
| 151 | HTLALNPQTI LSTIHVRQAA LTALFTYLRQ DVGSCFATAP |
| | AILIHQEYPE |
| 201 | EFLKDLNDLI SSGKLSRIVN QREIAVPINL SGCIGELFKP |
| | LRILDLYPDP |
| 251 | LVKLSSSPGL KKAFSAANIA ETLGDSEAQI QQLLSHQYLM |
| | QKLQNVHETL |
| 301 | TANDIIKSTL LHYYQLQEST VRAIFFKEGL FSKEQVAFST |
| | QHPRELSEIQ |
| 351 | RVYHYLHAYE EAKSAFIHDT QNPLLKAWEY TLATLADASQ |
| | PTISNHIRLA |
| 401 | LGWKSEDPUS LVSLVTHFVE EEVENIRILV QQCEQTYUEA |
| | RSQLEYIEGR |
| 451 | MRNPLNNQDS QILTMDHMRF RQELNKALYE WDSAQEKAKK |
| | FLHLPEFLLS |
| 501 | FYTKQIPLYE RSSYDAFIQE FAHLYANAPA GFRILFTHGR |
| | TEPNTWSPIY |
| 551 | SINEFIRFLS EFFTSTESEL LGKHAVINLE KETSRLVHNI |
| | TAMLHTDVFQ |
| 601 | EALLTRILEA YQLPVPPSIL NHLDQLSQTP WVYVSGGTVD |
| | TLLLDYFESS |
| 651 | EPLTLTEKHP ENPHELAAFY ADALKDLPTG IKSYLEEGSH |
| | SLLSSSPTHV |
| 701 | FSIIAGSPLF REAWDNDWYS YTWLRDVWVK QHQDFLQDTI |
| | LPQLSIYAFI |
| 751 | ENFCNKYALQ HVVHDFHDFC SDHSLTLPEL YDKGSRFLSS |
| | LFTKDKTVAL |
| 801 | IYIRRLLYLM VREVPYVSEQ QLPEVLDNVS SYLGISSRIT |
| | YEKFRSLIEE |
| 851 | TIPKMTLLSS ADLRHIYKGL LMQSYQKITY EEDTYLRLTT |
| | AMRHHNLAYP |
| 901 | APLLFADSNW PSIYFGFILM PGTTEIDLWK FNYAGLQGQP |
| | LDNIQELEAT |
| 951 | SRPWTLYANP IDYGMPPPPG YRSPLPKBFF * |

The cp7101 nucleotide sequence <SEQ ID 124> is:

| | |
|---|---|
| 1 | ATGTATTCGT GTTACAGCAA AGGAATATCC CATAACTATC |
| | TTCTACATCC |
| 51 | TATGTCACGT TTGGATATTT TTGTTTTCGA TTCTCTGATC |
| | GCAAACCAGG |
| 101 | ATCAAAATCT TCTTGAGGAA ATTTTCTGTT CTGAAGACAC |
| | AGTTTTATTT |
| 151 | AAAGCCTACC GTACTACGGC TCTACATTCC CCTCTAGCTG |
| | CTAAGAACCT |
| 201 | AAATATCGCC CGTAAAGTCG CAAATTATAT CTTAGCTGAC |
| | AATGGGGAAA |
| 251 | TCGATACAGT AAAGCTTGTC GAAGCCATTC ACCATCTCTC |
| | ACAATGTACC |
| 301 | TATCCTTTAG GGCCTCATCG CCATAATGAA GCTCAAGATC |
| | GTGAACACCT |
| 351 | CCTTAAAATG CTAAAAGCTC TAAAGGAAAA TCCTAAATTA |
| | AAAGAAAGCA |
| 401 | TCAAAACTCT CTTTGTCCCT TCATACTCTA CAATCCAAAA |
| | CCTAATTCGC |
| 451 | CATACACTAG CATTGAATCC ACAGACAATT CTCTCTACGA |
| | TTCATGTGCG |
| 501 | TCAAGCAGCA CTCACAGCGC TCTTCACCTA CCTTCGGCAA |
| | GATGTAGGTT |
| 551 | CCTGTTTTGC TACGGCTCCT GCCATTCTCA TTCACCAAGA |
| | ATATCCAGAA |
| 601 | CGATTCCTTA AAGATCTCAA TGATCTCATT AGCAGTGGCA |
| | AACTCTCTAG |
| 651 | AATCGTAAAC CAAAGGGAAA TTGCGGTTCC TATAAACCTT |
| | TCGGGATGCA |
| 701 | TTGGAGAGCT ATTCAAGCCT TTAAGGATTC TAGATCTTTA |
| | TCCTGATCCT |
| 751 | CTGGTTAAGC TCTCCTCATC TCCAGGACTC AAAAAAGCCT |
| | TTTCTGCTGC |
| 801 | CAATCTTATT GAAACTCTTG GGGATTCTGA AGCACAAATC |
| | CAACAGTTGC |
| 851 | TCTCGCATCA ATATTTGATG CAAAAACTAC AAAATGTCCA |
| | TGAGACCTTA |
| 901 | ACTGCTAACG ACATTATCAA ATCGACACTT CTGCACTACT |
| | ATCAGCTCCA |
| 951 | AGAAAGTACT GTACGAGCTA TTTTCTTCAA AGAAGGGTTG |
| | TTCAGCAAAG |
| 1001 | AACAAGTGGC ATTCTCGACG CAACACCCCA GAGAGCTCTC |
| | AGAAATACAA |
| 1051 | CGGGTATACC ACTACTTACA TGCCTATGAA GAAGCAAAAT |
| | CTGCTTTTAT |
| 1101 | CCATGACACT CAAAATCCCT TACTGAAAGC CTGGGAGTAT |
| | ACTTTAGCGA |
| 1151 | CTCTTGCGGA TGCTAGCCAA CCTACCATCT CAAACCATAT |
| | CCGCCTTGCC |
| 1201 | TTAGGATGGA AAAGTGAAGA CCCTCACAGT CTTGTATCTC |
| | TAGTTACACA |
| 1251 | CTTTGTTGAA GAGGAAGTAG AAAACATCCG AATTTTAGTC |
| | CAACAATGTG |
| 1301 | AACAGACCTA TCACGAAGCA CGCTCCCAAC TAGAATATAT |
| | TGAAGGGCGG |
| 1351 | ATGCGCAACC CACTAAATAA TCAAGACAGT CAGATTTTGA |
| | CGATGGATCA |
| 1401 | CATGCGCTTC CGTCAAGAAC TCAATAAAGC TCTTTATGAG |
| | TGGGATAGTG |
| 1451 | CTCAAGAAAA GGCAAAGAAA TTTCTACATC TTCCTGAATT |
| | CTTACTTTCT |
| 1501 | TTCPATACAA AGCAAATTCC CTTATACTTT CGTAGTTCTT |
| | ACGATGCCTT |
| 1551 | CATTCAAGAA TTTGCTCATC TCTATGCTAA TGCTCCCGCT |
| | GGCTTCCGTA |
| 1601 | TTCTTTTCAC GCATGGACGC ACCCATCCGA ACACATGGTC |
| | CCCCATCTAT |
| 1651 | TCGATTAATG AATTTATACG TTTTCTTTCT GAATTCTTCA |
| | CCTCCACAGA |
| 1701 | GTCAGAACTT CTGGGGAAAC ATGCCGTGAT CAATTTAGAG |
| | AAAGAAACAT |
| 1751 | CTCGGCTCGT CCACAACATC ACTGCCATGC TACACACGGA |
| | TGTTTTCCAA |
| 1801 | GAAGCTCTCC TTACAAGAAT TTTAGAAGCC TATCAGCTTC |
| | CTGTGCCTCC |
| 1851 | CTCCATCTTA AACCACTTAG ATCAGCTGTC ACAAACTCCC |
| | TGGGWTTATG |
| 1901 | TTTCTGGAGG AACAGTGGAC ACTCTTCTTT TGGATTATTT |
| | TGAAAGCTCA |
| 1951 | GAACCTCTGA CACTTACAGA AAAGCATCCT GAAAATCCTC |
| | ATGAGCTTGC |
| 2001 | AGCTTTCTAC GCAGACGCCC TTAAAGATCT CCCTACAGGA |
| | ATTAAAAGTT |
| 2051 | ATCTAGAAGA AGGATCCCAC TCTCTACTTA GCTCATCACC |
| | CACCCACGTT |
| 2101 | TTCTCTATAA TCGCAGGATC TCCTTTATTT CGGQAAGCTT |
| | GGGATAATGA |
| 2151 | TTGGTACAGC TATACCTGGC TTCGTGATGT CTGGGTGAAA |
| | CAACACCAAG |
| 2201 | ATTTCCTTCA AGATACTATA TTACCTCAGC TAAGTATCTA |
| | TGCTTTCATA |
| 2251 | GAGAATTTTT GTAACAAATA TGCTTTGCAA CATGTAGTTC |
| | ATGACTTTCA |
| 2301 | TGATTTCTGC TCCGACCACT CCTTGACTCT TCCGGAGCTC |
| | TATGACAAAG |
| 2351 | GATCGCGTTT TCTAAGCTCC TTATTCACCA AAGATAAGAC |
| | CGTAGCTCTT |
| 2401 | ATCTATATAC GCCGTCTTCT CTACCTTATG GTCCGTGAAG |
| | TCCCTTATGT |
| 2451 | TTCAGAACAA CAGCTTCCAG AAGTCTTRGA TAACGTCTCT |
| | TCATATCTCG |
| 2501 | GGATTTCCTC TCGTATTACC TATGAGAAAT TCCGCTCCCT |
| | GATAGAGGAA |
| 2551 | ACCATCCCTA AAATGACCTT ACTCTCCTCA GCAGACCTGA |
| | GGCATATCTA |
| 2601 | TAAAGGTCTC CTCATGCAAA GTTATCAAAA GATCTACACC |
| | GAAGAAGATA |
| 2651 | CGTACCTCCG CCTCACCACG GCAATGAGGC ATCATAATCT |
| | TGCCTATCCC |
| 2701 | GCTCCTTTGC TCTTTGCAGA CAGTAACTGG CCTTCTATTT |
| | ATTTTGGATT |
| 2751 | CATCCTAAAT CCAGGAACCA CAGAGATCGA TCTTTGGAAA |
| | TTTAACTATG |
| 2801 | CAGGGCTGCA AGGACAGCCT CTTGACAATA TCCAGGAGCT |
| | GTTCGCAACG |
| 2851 | TCAAGACCCT GGACCCTCTA TGCAAATCCT ATAGATTATG |
| | GCATGCCACC |
| 2901 | GCCTCCAGGC TACCGCAGCC GCCTCCCTAA AGAATTTTTC |
| | TAG |

The PSORT algorithm predicts a cytoplasmic location (0.206).

The protein was expressed in E. coli and purified as a GST-fusion (FIG. 62A) or his-tagged product. The proteins were used to immunise mice, whose sera were used in Western blot (FIG. 62B) and FACS (FIG. 62C) analyses.

This protein also showed good cross-reactivity with human sera, including sera from patients with pneumonitis.

These experiments show that cp7101 is a surface-exposed and immunoaccessible protein, and that it is a useful immunogen. These properties are not evident from the sequence alone.

### Example 63

The following C. pneumoniae protein (PID 4377107) was expressed <SEQ ID 125; cp7107>:

| | |
|---|---|
| 1 | MSIVRTSALP LPCLSRSETF KKVRSHMKFM KVLTPWIYRK |
| | DLWVTAFLLT |
| 51 | AIPGSFARTL VDIAGEPRHA AQATGVSGDG KIVIGMKVPD |
| | DPFAITVGPQ |
| 101 | YIDGHLQPLE AVRPQOSVYP NGITPDGTVI VGTNYAIGMG |
| | SVAVKWVNGI |
| 151 | VSELPMLPDT LDSVASAVSA DGRVIGGNRN XNLGASVAVK |
| | WEDDVITQLP |
| 201 | SLPDAMNACV NGISSDGSII VGTMVDVSWR NTAVQWIGDQ |
| | LSVIGTLGGT |
| 251 | TSVASAISTD GTVIVGGSEN ADSQTHAYAY KNGVMSDIGT |
| | LGGFYSLAHA |
| 301 | VSSDGSVIVG VSTNSEHRYH AFQYADGQMV DLGTLGGPES |
| | YAQGVSDGDK |
| 351 | VIVGKAQVPS GDWHAPLCPF QAPSPAPVHG GSTVVTSQNP |
| | RGMVDINATY |
| 401 | SSLKNSQQQL QRLLIQHSAK VESVSSGAPS PTSVKGAISK |
| | QSPAVQNDVQ |
| 451 | KGTFLSYRSQ VHGNVQNQQL LTGAFMLWKL ASAPKCGFKV |
| | ALHYGSQDAL |
| 501 | VERAALPYTE QGLGSSVLSG FGGQVQGRYD FNLGETVVLQ |
| | PFMGIQVLHL |
| 551 | SREGYSEKNV RFPVSYDSVA YSAATSFNGA RVFASLSPKM |
| | STAATLGVER |
| 601 | DLNSHIDEFK GSVSAMGNFV LENSTVSVLR PFASLAMYYD |
| | VRQQQLVTLS |
| 651 | VVMNQQPLTG TLSLVSQSSY NLBF* |

The cp7107 nucleotide sequence <SEQ ID 126> is:

| | |
|---|---|
| 1 | ATGAGTATAG TCAGAAATTC TGCATTGCCA CTTCCGTGTT |
| | TAAGCAGATC |
| 51 | CGAAACCTTT AAAAAAGTTA GGTCGCATAT GAAATTTATG |
| | AAAGTCCTTA |
| 101 | CTCCATGGAT TTATCGAAAA GATCTTTGGG TAACAOCATT |
| | CTTACTGACA |
| 151 | GCAATTCCAG GATCTTTTGC ACATACTCTT GTTGATATAG |
| | CAGGAGAACC |
| 201 | TCGGCATGCT GCTCAAGCAA CAGGAGTTTC TGGAGATGGT |
| | AAAATTGTTA |
| 251 | TAGGAATGAA AGTTCCGGAT GATCCTTTTG CTATAACTGT |
| | AGGATTTCAA |
| 301 | TATATTGATG GGCATTTGCA ACCCTTAGAG GCAGTACGTC |
| | CTCAATGCTC |
| 351 | TGTATACCCT AATGGTATAA CCCCGGACGG AACGGTTATT |
| | GTGGGTACAA |
| 401 | ACTATCCCAT CGGGATGGGT AGTGTTGCTG TGAAATGGGT |
| | AAATGGCAAG |
| 451 | GTTTCTGAAC TTCCCATGCT CCCTGACACC CTCGATTCTG |
| | TAGCATCGGC |
| 501 | AGTTTCTGCA GATGGAAGAG TGAPTGGAGG GAATAGAAAT |
| | ATAAATCTTG |
| 551 | GCGCTTCTGT TGCTGTGAAA TGGGAGGACG ACGTGATTAC |
| | ACAACTTCCT |
| 601 | TCTCTTCCTG ATGCTATCAA TGCTTGTGTT AACCGAATTT |
| | CTTCAGATGG |
| 651 | TTCTATAATT GTAGGAACCA TGGTAGACGT GTCATGGAGA |
| | AATACCGCAG |
| 701 | TACAATGGAT CGGGGATCAG CTCTCTGTTA TTGGGACTTT |
| | AGGAGGAACT |
| 751 | ACTTCTGTTG CTAGTGCAAT CTCAACAGAT GGCACTGTGA |
| | TTGTAGGAGG |
| 801 | TTCTGAAAAT GCAGATTCTC AGACTCATGC CTATGCTTAT |
| | AAAAACGGTG |
| 851 | TTATGAGCGA TATAGGGACC CTCGGAGGTT TTTATTCTTT |
| | AGCACATGCA |
| 901 | GTATCTTCAG ATGGTTCTGT GATTGTAGGA GTATCCACGA |
| | ACTCTGAGCA |
| 951 | TAGATATCAT GCATTCCAAT ATGCTGATGG ACAGATGGTA |
| | GATTTAGGAA |
| 1001 | CTTTAGGAGG GCCTGAATCT TATGCTCAAG GTGTGTCTGG |
| | AGATGGAAAG |
| 1051 | GTAATTGTGG GTACAGCACA AGTACCATCT GGAGATTGGC |
| | ATGCGTTCCT |
| 1101 | ATGTCCTTTC CAAGCTCCGA GCCCTGCTCC TGTCCATGGG |
| | GGAAGCACTG |
| 1151 | TCGTAACTAG CCAGAATCCA CGTGGAATGG TAGATATCAA |
| | TGCTACCTAC |
| 1201 | TCCTCTTTGA AAAATAGCCA ACAACAACTA CAAAGATTGC |
| | TTATCCAGCA |
| 1251 | TAGTGCAAAA GTTGAAAGTG TATCCTCAGG AGCACCATCT |
| | TTTACAAGTG |
| 1301 | TGAAAGGTGC GATCTCAAAA CAGAGCCCTG CAGTGCAAAA |
| | TGATGTACAG |
| 1351 | AAAGGGACGT TTTTAAGTTA CCGTTCCCAA GTTCATGGAA |
| | ACGTGCAGAA |
| 1401 | TCAGCAATTG CTCACAGGAG CTTTTATGGA CTGGAAACTC |
| | GCTTCAGCTC |
| 1451 | CTAAATGCGG CTTTAAAGTA OCTOTOCACT ATGGCTCTCA |
| | AGATGCTCTC |
| 1501 | GTAGAACGTG CAGCTCTTCC TTACACAGAA CAAGGCTTAG |
| | GAAGCAGTGT |
| 1551 | CTTCTCAGGT TTTGGAGGAC AAGTTCAAGG ACGCTATGAC |
| | TTTAATTTAG |
| 1601 | GAGAAACTGT TGTTCTGCAA CCCTTTATGG GCATTCAAGT |
| | TCTCCACCTA |
| 1651 | AGTAGAGAAG GGTATTCTGA GAAGAATGTT CGATTTCCTG |
| | TAAGCTATGA |
| 1701 | TTCTGTAGCC TACTCAGCAG CTACTAGCTT TATGGGTGCG |
| | CATGTATTTG |
| 1751 | CCTCCCTAAG CCCTAAAATG AGTACAGCAG CAACTTTAGG |
| | TGTGGAGAGA |
| 1801 | GATCTGAATT CACATATAGA TGAATTTAAG GGATCCGTCT |
| | CTGCTATGGG |
| 1851 | AAACTTTGTC TTGGAAAATT CTACAGTGAG TGTTTTAAGA |
| | CCTTTTGCTT |
| 1901 | CTCTTGCTAT GTACTATGAC GTAAGACAAC AGCAACTCGT |
| | GACGTTGTCA |
| 1951 | GTAGTTATGA ATCAACAACC CTTAACAGGC ACACTAAGCT |
| | TAGTAAGCCA |
| 2001 | AAGTAGCTAT AATCTTAGCT TCTAA |

The PSORT algorithm predicts an inner membrane location (0.100).

The protein was expressed in E. coli and purified as a GST-fusion (FIG. 63A) or his-tagged product. The proteins were used to immunise mice, whose sera were used in Western blot (FIG. 63B) and FACS (FIG. 63C) analyses.

These experiments show that cp7107 is a surface-exposed and immunoaccessible protein, and that it is a useful immunogen. These properties are not evident from the sequence alone.

### Example 64

The following C. pneumoniae protein (PID 4376467) was expressed <SEQ ID 127; cp6467>:

| | |
|---|---|
| 1 | MLRFFAVFIS TLWLITSGCS PSQSSKGFV VNMKEMPRSL |
| | DPGKTRLIAD |
| 51 | QTLMRHLYEG LVEEHSQNGE IKPALAESYT ISEDGTRYTF |
| | KIKNILWSNG |
| 101 | DPLTAQDFVS SWKEILKEDA SSVYLYAFLP IKNAEAIFDD |
| | TESPENLGVR |
| 151 | ALDKRHLEIQ LETPCAHFLH FLThPIFFPV HETLRNYSTS |
| | FEEMPITCGA |
| 201 | PRPVSLERGL RLBLBKNPMY HNKSRVKLHK IIVQFISNAN |
| | TAAILFKHKK |
| 251 | LDWQGPPWGE PIPPEXSABL HQDDQLFSLP GASTTWLLFN |
| | IQKKPWNNAR |
| 301 | LRKALSLAID KDMLTKVVYQ GLAEPTDHIL HPRLYPGTYP |
| | ERKRQNERIL |
| 351 | EAQQLFEEAL VELQMTREDL EKETLTFSTF SFSYGRICQM |
| | LREQWKKVLR |
| 401 | FTIPIVGQEF FTIQKNFLEG NYSLTVNQWT AAFIDPMSYL |
| | MIFANPGGIS |
| 451 | PYHLQDSHFQ TLLIKITQEH KKHLRNQLII EALDYLEHCH |
| | ILEPLCHPNL |
| 501 | RIALNKNIKN FNLFVRRTSD FRFIEKL* |

A predicted signal peptide is highlighted.

The cp6467 nucleotide sequence <SEQ ID 128> is:

| | |
|---|---|
| 1 | ATGCTCCGTT TCTTCGCTGT ATTTATATCA ACTCTTTGGC |
| | TCATTACCTC |
| 51 | AGGATGTTCC CCATCCCAAT CCATCCCAAT AATTTTTGTG |
| | GTAAATATGA |
| 101 | AGGAAATGCC ACGCTCCTTG GATCCTGGAA AAACTCGTCT |
| | CATTCCAGAC |
| 151 | CAAACTCTAA TGCGTCATCT ATATGAAGGA CTCGTCGAAG |
| | AACATTCCCA |
| 201 | AAATGGAGAG ATTAAACCAG CCCTTGCAGA AAGCTACACC |
| | ATCTCCGAAG |
| 251 | ACGGGACTCG GTACACATTT AAAATCAAAA ACATCCTTTG |
| | GAGTAACGGA |
| 301 | GACCCTCTGA CAGCTCAAGA CTTTGTCTCC TCTTGGAAGG |
| | AAATCCTAAA |
| 351 | GGAAGATGCG TCCTCCGTAT ATCTCTATGC GTTTTTACCT |
| | ATCAAAAATG |
| 401 | CTCGGGCAAT CTTTGATGAT ACTGAGTCTC CAGAAAATCT |
| | AGGAGTCCGA |
| 451 | GCTTTAGATA AGCGTCATCT CGAAATTCAG TTAGAAACTC |
| | CCTGCGCGCA |
| 501 | TTTCCTACAT TTCTTGACTC TTCCTATTTT TTTCCCTGTT |
| | CATGAAACTC |
| 551 | TGCGAAACTA TAGCACCTCT TTTGAAGAGA TGCCCATTAC |
| | CPGCGGTCCT |
| 601 | TTCCGCCCPG TGTCTCTAGA AAAAGGCCTG AGACTCCATC |
| | TAGAGAAAAA |
| 651 | CCCTATGTAC CATAATAAAA GCCGTGTGAA ACTACATAAA |
| | ATTATTGTAC |
| 701 | AGTTTATCTC AAACGCTAAC ACTGCAGCCA TTCTATTCAA |
| | ACATAAGAAA |
| 751 | TTAGATTGGC AAGGACCTCC TTGGGGAGAA CCTATCCCTC |
| | CAGAAATCTC |
| 801 | AGCPTCTCTA CATCAAGATG ACCAGCTCTT TTCTCTTCCG |
| | GGCGCTTCGA |
| 851 | CTACATGGTT ACTCTTTAAT ATACAAAAAA AACCTTGGAA |
| | CAATGCTAAA |
| 901 | TTACGCAAGG CATTGAGCCT TGCAATAGAC AAAGATATGT |
| | TAACCAAAGT |
| 951 | GGTATACCAA GGTCTTGCAG AACCTACAGA TCATATCCTA |
| | CATCCAACAC |
| 1001 | TTTATCCAGG GACCTATCCC GAACGGAAAA GACAAAACGA |
| | AAGAATTCTT |
| 1051 | GAGGCTCAAC AACTCTTTGA AGAAGCTCTA GACGAACTTC |
| | AAATGACACG |
| 1101 | CGAAGATCTA GAAAAGGAAA CTTTGACTTT CTCAACCTTT |
| | TCTTTTTCTT |
| 1151 | ACGGAAGGAT TTGCCAAATG CTAAGAGAAC AATGGAACAA |
| | AGTCTTAAAA |
| 1201 | TTTACTATCC CTATAGTAGG CCAAGAGTTT TTCACAATAC |
| | AAAAAAACTT |
| 1251 | CCTAGAGGGG AACTATTCCC TAACCGTGAA CCAATGGACC |
| | GCAGCAATTA |
| 1301 | TTGATCCGAT GTCTTATCTC ATGATCTTTG CCAATCCTGG |
| | AGGAATTTCC |
| 1351 | CCCTATCACC TCCAAGATTC ACACTTTCAA ACTCTTCTCA |
| | TAAAGATCAC |
| 1401 | TCAAGAACAT AAAAAACACC TACGAAATCA GCTTATTATT |
| | GAAGCCCTTG |
| 1451 | ACTATTTAGA ACACTGTCAC ATTCTCGAAC CACTATGTCA |
| | TCCAAATCTT |
| 1501 | CGAATTGCTT TGAACAAAAA CATTAAAAAC TTTAATCTTT |
| | TTGTTCGACG |
| 1551 | AACTTCAGAC TTTCGTTTTA TAGAAAAACT ATAG |

The PSORT algorithm predicts an outer membrane lipoprotein (0.790).

The protein was expressed in E. coli and purified as a his-tag product and a GST-fusion protein, as shown in FIG. 64A. The recombinant his-tag protein was used to immunise mice, whose sera were used in a Western blot (FIG. 64B). The recombinant GST-fusion protein was also used to immunise mice, whose sera were used in a Western blot (FIG. 64C) and for FACS analysis (FIG. 64D).

These experiments show that cp6467 is a surface-exposed and immunoaccessible protein, and that it is a useful immunogen. These properties are not evident from the sequence alone.

### Example 65

The following C. pneumoniae protein (PID 4376679) was expressed <SEQ ID 129; cp6679>:

| | |
|---|---|
| 1 | MRKMLVLLAS LGLLSPTLSS CTHLGSSGSY HPKLYTSGSK TKGVIAMLPV |
| 51 | FHRPGKSLEP LPWNLQGEFT EEISKKFYAS EKVFLIKHNA SPQTVSQFYA |
| 101 | PIANRLPETI IEQFLPAEFI VATELLEQKT GKEAGVDSVT ASVRVRVFDI |
| 151 | RHHKIALIYQ EIIECSQPLT TLVNDYHRYG WNSKHFDSTP MGLMHSRLFR |
| 201 | EVVARVEGYV CANYS* |

A predicted signal peptide is highlighted.

The cp6679 nucleotide sequence <SEQ ID 130> is:

| | |
|---|---|
| 1 | ATGCGAAAAA TGTTGGTATT ATTG~CATCT TTAGGACTTC TATCCCCAAC |
| 51 | CCTATCCAGC TGCACTCACT TAGGCTCTTC AGGAAGTTAT CATCCTAAGC |
| 101 | TATACACTTC AGGGAGCAAA ACTAAAGGTG TGATTGCGAT GCTTCCTGTA |
| 151 | TTTCATCGCC CAGGAAAGAG TCTTGAACCT TTACCTTGGA ACCTCCAAGG |
| 201 | AGAATTTACT GAAGAGATCA GCAAAAGGTT TTATGCTTCG GAAAAGGTCT |
| 251 | TCCTGATCAA GCACAATGCT TCACCTCAGA CAGTCTCTCA GTTCTATGCT |
| 301 | CCGATTGCGA ATCGTCTACC CGAAACAATT ATTGAGCAAT TTCTTCCTGC |
| 351 | AGAATTCATT GTTGCTACAG AACTGTTAGA ACAAAAGACA GGGAAAGAAG |
| 401 | CAGGTGTCGA TTCTGTAACA GCGTCTGTAC GTGTTCGCGT TTTTGATATC |
| 451 | CGTCATCATA AAATAGCTCT CATTTATCAA GAGATTATCG AATGCAGCCA |
| 501 | GCCTTTAACT ACCCTAGTCA ATGATTATCA TCGCTATGGC TGGAACTCAA |
| 551 | AACATTTTGA TTCAACGCCC ATGGGCTTAA TGCATAGCCG TCTTTTCCGC |

The PSORT algorithm predicts an inner membrane location (0.149).

The protein was expressed in E. coli and purified as a his-tag product (FIG. 65A) and as a GST-fusion product (FIG. 65B). The recombinant protein was used to immunise mice, whose sera were used in a Western blot (FIG. 65C) and for FACS analysis.

These experiments show that cp6679 is a surface-exposed and immunoaccessible protein, and that it is a useful immunogen. These properties are not evident from the sequence alone.

### Example 66

The following C. pneumoniae protein (PID 4376890) was expressed <SEQ ID 131; cp6890>:

| | |
|---|---|
| 1 | MKQLLFCVCV FAMSCSAYAS PRRQDPSVMK ETFRNNYGII VSGQEWVKRG |
| 51 | SDGTITKVLK NGATLHEVYS GGLLHGEITL TFPHTTALDV VQIYDQGRLV |
| 101 | SRKTFFVNGL PSQEELFNED GTFVLTRWPD NNDSDTITKP YFIETTYQGH |
| 151 | VIEGSYTSFN GKYSSSIHNG EGVRSVFSSN NILLSEETFN EGVMVKYTTF |
| 201 | YPNRDPESIT HYQNGQPHGL RLTYLQGGIP NTIEEWRYGF QDGTTIVFKN |
| 251 | GCKTSEIAYV KGVKEGLELR YNEQEIVAEE VSWRNDFLHG ERKIYAGGIQ |
| 301 | KHEWYYRGRS VSKAKFERLN AAG* |

A predicted signal peptide is highlighted.

The cp6890 nucleotide sequence <SEQ ID 132> is:

| | |
|---|---|
| 1 | ATGAAACAAT TACTTTTCTG TGTTtGCGTA TTTGCTATGT CATGTTCTGC |
| 51 | TTACGCATCC CCACGACGAC AAGATCCTTC TGTTATGAAG GAAACATTCC |
| 101 | GAAATAATTA TGGCATTATT GTTTCCGGTC AAGAATGGGT AAAGCGTGGT |
| 151 | TCTGACGGCA CCATCACCAA AGTACTCAAA AATOGAGCTA CCCTGCATGA |
| 201 | AGTTTATTCT GGAGGCCTCC TTCATGGGGA AATTACCTTA ACGTTTCCCC |
| 251 | ATACCACAGC ATTGGACGTT GTTCAAATCT ATGATCAAGG TAGACTCGTT |
| 301 | TCTCGCAAAA CCTTTTTTGT GAACGGTCTT CCATCTCAAG AAGAGCTGTT |
| 351 | CAATGAAGAT GGCACGTTTG TCCTCACACG ATGGCCGGAC AACAACGACA |
| 401 | GTGATACCAT CACAAAGCCT TACTTCATAG AAACGACATA TCAAGGGCAT |
| 451 | GTCATAGAAG GAAGTTATAC TTCCTTTAAT GGGAAATACT CCTCATCCAT |
| 501 | CCACAATGGA GAGGGAGTTC GTTCTGTGTT CTCCTCCAAT AACATCCTTC |
| 551 | TTTCTGAAGA GACCTTCAAT GAAGGTGTCA TGGTGAAATA TACCACATTC |
| 601 | TATCCGAATC GCGATCCCGA ATCGATTACT CATTATCAAA ATGGACAGCC |
| 651 | TCACGGCTTA CGGCTAACAT ATCTACAAGG TGGCATCCCC AATACGATAG |
| 701 | AGGAGTGGCG TTATGGCTTT CAAGACGGAA CGACCATCGT ATTTAAAAAT |
| 751 | GGTTGTAAGA CATCTGAGAT CGCTTATGTT AAGGGAGTGA AAGAAGGTTT |
| 801 | AGAACTGCGC TACAATGAAC AGGAAATTGT AGCTGAAGAA GTTTCTTGGC |
| 851 | GTAATGATTT TCTGCATGGA GAACGTAAGA TCTATGCTGG AGGAATCCAA |
| 901 | AAGCATGAAT GGTATTACCG CGGGAGATCT GTATCTAAAG CCAAATTCGA |
| 951 | GCGGCTAAAT GCTGCAGGAT AG |

The PSORT algorithm predicts an outer membrane location (0.940).

The protein was expressed in E. coli and purified as a GST-fusion product, as shown in FIG. 66A. The recombinant protein was used to immunise mice, whose sera were used in a Western blot (FIG. 66B) and for FACS analysis. A his-tagged protein was also expressed.

These experiments show that cp6890 is a surface-exposed and immunoaccessible protein, and that it is a useful immunogen. These properties are not evident from the sequence alone.

### Example 67

The following C. pneumoniae protein (PID 6172323) was expressed <SEQ ID 133; cp0018>:

| | |
|---|---|
| 1 | MKTSVSMLLA LLCSGASSIV LHAATTPLNP EDGFIGEGNT NTFSPKSTTD |
| 51 | AAGTTYSLTG EVLYIDPGKG GSITGTCFVE TAGDLTFLGN GNTLKFLSVD |
| 101 | AGANIAVAHV QGSKNLSFTD FLSLVITESP KSAVTTGKGS LVSLGAVQLQ |
| 151 | DINTLVLTSN ASVEDGGVIK GNSCLIQGIK NSAIFGQNTS SKKGGAISTT |
| 201 | QGLTIENNLG TLKFNENKAV TSGGALDLGA ASTFTANHEL IFSQNKTSGN |
| 251 | AANGGAINCS GDLTFTDNTS LLLQENSTMQ DGGALCSTGT ISITGSDSIN |
| 301 | VIGNTSGQKG GAISAASLKI LGGQGGALFS NNVVTHATPL GGAIFINTGG |
| 351 | SLQLFTQGGD IVPEGNQVTT TAPNATTKRN VIHLESTAKW TGLAASQGNA |
| 401 | IYFYDPITTN DTGASDNLRI NEVSANQKLS GSIVFSGERL STAEAIAENL |
| 451 | TSRINQPVTL VEGSLVLKQG VTLITQGFSQ EPESTLLLDL GTSL* |

A predicted signal peptide is highlighted.

The cp0018 nucleotide sequence <SEQ ID 134> is:

| | |
|---|---|
| 1 | ATGAAGACTT CAGTTTCTAT GTTGTTGGCC CTGCTTTGCT CGGGGGCTAG |
| 51 | CTCTATTGTA CTCCATGCCG CAACCACTCC ACTAAATCCT GAAGATGGGT |
| 101 | TTATTGGGGA GGGCAATACA AATACTTTTT CTCCGAAATC TACAACGGAT |
| 151 | GCTGCAGGAA CTACCTACTC TCTCACAGGA GAGGTTCTGT ATATAGATCC |
| 201 | GGGGAAAGGT GGTTCAATTA CAGGAACTTG CTTTGTAGAA ACTGCTGGCG |
| 251 | ATCTTACATT TTTAGGTAAT GGAAATACCC TAAAGTTCCT GTCGGTAGAT |
| 301 | GCAGGTGCTA ATATCGCGGT TGCTCATGTA CAAGGAAGTA AGAATTTAAG |
| 351 | CTTCACAGAT TTCCTTTCTC TGGTGATCAC AGAATCTCCA AAATCCGCTG |
| 401 | TTACTACAGG AAAAGGTAGC CTAGTCAGTT TAGGTGCAGT CCAACTGCAA |
| 451 | GATATAAACA CTCTAGTTCT TACAAGCAAT GCCTCTGTCG AAGATGGTGG |
| 501 | CGTGATTAAA GGAAACTCCT GCTTGATTCA GGGAATCAAA AATAGTGCGA |
| 551 | TTTTTGGACA AAATACAPCT TCGAAAAAAG GAGGGGCGAT CTCCACGACT |
| 601 | CAAGGACTTA CCATAGAGAA TAACTTAGGG ACGCTAAAGT TCAATGAAAA |
| 651 | CAAAGCAGTG ACCTCAGGAG GCGCCTTAGA TTTAGGAGCC GCGTCTACAT |
| 701 | TCACTGCGAA CCATGAGTTG ATATTTTCAC AAAATAAGAC TTCTGGGAAT |
| 751 | GCTGCAAATG GCGGAGCCAT AAATTGCTCA GGGGACCTTA CATTTACTGA |
| 801 | TAACACTTCT TTGTTACTTC AAGAAAATAG CACAATGCAG GATGGTGGAG |
| 851 | CTTTGTGTAG CACAGGAACC ATAAGCATTA CCGGTAGTGA TTCTATCAAT |
| 901 | GTGATACGAA ATACTTCAGG ACAAAAAGGA GGAGCGATTT CTGCAGCTTG |
| 951 | TCTCAAGATT TTGGGAGGGC AGGGAGGCGC TCTCTTTTCT AATAACGTAG |
| 1001 | TGACTCATGC CACCCCTCTA GGAGGTGCCA TTTTTATCAA CACAGGAGGA |
| 1051 | TCCTTGCAGC TCTTCACTCA AGGAGGGGAT ATCGTATTCG AGGGGAATCA |
| 1101 | GGTCACTACA ACAGCTCCAA ATGCTACCAC TAAGAGAAAT GTAATTCACC |
| 1151 | TCGAGAGCAC CGCGAAGTGG ACGGGACTTG CTGCAAGTCA AGGTAACGCT |
| 1201 | ATCTATTTCT ATGATCCCAT TACCACCAAC GATACGGGAG CAAGCGATAA |
| 1251 | CTTACGTATC AATGAGGTCA GTGCAAATCA AAAGCTCTCG GGATCTATAG |
| 1301 | TATTTTCTGG AGAGAGATTG TCGACAGCAG AAGCTATAGC TGAAAATCTT |
| 1351 | ACTTCGAGGA TCAACCAGCC TGTCACTTTA GTAGAGGGGA GCTTAGTACT |
| 1401 | TAAACAGGGA GTGACCTTGA TCACACAAGG ATTCTCGCAG GAGCCAGAAT |
| 1451 | CCACGCTTCT TTTGGATCTG GGGACCTCAT TATAA |

The PSORT algorithm predicts outer membrane (0.935).

The protein was expressed in E. coli and purified as a GST-fusion product (FIG. 67A). The recombinant protein was used to immunise mice, whose sera were used in a Western blot (FIG. 67B) and for FACS analysis.

These experiments show that cp0018 is a surface-exposed and immunoaccessible protein, and that it is a useful immunogen. These properties are not evident from the sequence alone.

### Example 68

The following C. pneumoniae protein (PID 4376262) was expressed <SEQ ID 135; cp6262>:

| | |
|---|---|
| 1 | MRKLRILAIV LIALSIILIA GGVVLLTVAI PGLSSVISSP AGMGACALGC |
| 51 | VMLALGIDVL LKKREVPIVL ASVTTTPGTG SPRSGISISG ADSTIRSLPT |
| 101 | YLLDEGHPQS MRKLRILAIV LIVFSIILIA SGVVLLTVAI PGLSSVISSP |
| 151 | AGMGACALGC VMLALGIDVL LKKREVPIVL ASVTTTPGTG SPRSGISISG |
| 201 | ADSTIRSLPT YPLDEGHPQS MRKLRILAIV LIVFSIILIA SGVVLLTVAI |
| 251 | PGLSSIISSP AEMGACALGC VMLALGIDVL LKKREVPIVV PAPIPEEVVI |
| 301 | DDIDEESIRL QQEAEAALAR LPEEMSAFEG YIKVVESHLE NMKSLPYDGH |
| 351 | GLEEKTKHQI RVVRBSLKAM VPEPLDIRRI FEEEEFFFLS ARKRLIDLAT |
| 401 | TLVERKILTE QLERNNLRKA FSYLYQDSIF KKIIDNFEKL AWKFMILSKS |
| 451 | ICRFTIIFEN HEHGVAKSLL HKNAVLLEKV IYRSLQKSYR DIGMSSAKMK |
| 501 | ILHGNPFFSL EDNKKTIMKE HAEMLESLSS YRKVFLALSD ENVVDTPSDP |
| 551 | KKWDLSGIPC RDALSEISRD EQWQKKAHLK HQESLYTQAR DRLTDQSSKE |
| 601 | NQKELEKAEQ EYISSWERVK KFEIERVQER IRAIQKLYFN ILEREEETTG |
| 651 | QETVTPTVQG TTASSDLTDI LGRIEVSSRE DNQNQESCVK VLRSHEVEMS |
| 701 | WEVKQEYGPK KKEFQDQMGS LERFFTEHIE ELEVLQKDYS KHLSYFKKVN |
| 751 | NKKEVQYAKF RLKVLESDLE GILAQTESAE SLLTQEELPI LATRGALEKA |
| 801 | VFKGSLCCAL ASKAKPYFEE DPRPQDSDTQ LRALTLRLQE AKASLEEEIK |
| 851 | RFSNLENDIA EERRLLKESK QTEERAGLGV LREIAVESTY DLRSLTNTWE |
| 901 | GTPESEKVYF SMYLNYYNEE KRRAKTRLVE MTQRYRDFKH ALEAMQFNEE |
| 951 | ALLQEELSIQ APSE* |

A predicted signal peptide is highlighted.

The cp6262 nucleotide sequence <SEQ ID 136> is:

| | |
|---|---|
| 1 | ATGAGGAAAC TTCGTATTCT TGCGATCGTT CTCATAGCTT TGAGCATTAT |
| 51 | TTTGATTGCA GGTGGTGTGG TATTGCTTAC TGTAGCGATC CCTGGATTAA |
| 101 | GTTCAGTCAT TTCTTCCCCG GCAGGGATGG GTGCCTGTGC TTTGGGATGT |
| 151 | GTGATGCTTG CTTTAGGGAT CGATGTTCTT CTGAAGAAAC GAGAAGTCCC |
| 201 | TATAGTTCTC GCATCTGTAA CTACGACACC AGGAACTGGC AGCCCTAGAA |
| 251 | GTGGTATTTC TATTTCAGGA GCTGATAGCA CCATACGTTC TCTTCCTACG |
| 301 | TATCTCTTGG ACGAGGGACA TCCACAATCC ATGAGGAAAC TTCGTATTCT |
| 351 | TGCGATCGTT CTCATAGTTT TTAGCATTAT TTTGATTGCA AGTGGTGTGG |
| 401 | TATTGCTTAC TGTAGCGATC CCTGGATTAA GTTCAGTCAT TTCTTCCCCG |
| 451 | GCAGGGATGG GTGCCTGTGC TTTGGGATGT GTGATGCTTG CTTTAGGGAT |
| 501 | CGATGTTCTT CTGAAGAAAC GAGAAGTCCC TATAGTTCTC GCATCTGTAA |
| 551 | CTACGACACC AGGAACTGGC AGCCCTAGAA GTGGTATTTC TATTTCAGGA |
| 601 | GCTGATAGCA CCATACGTTC TCTTCCTACG TATCCCTTGG ACGAGGGACA |
| 651 | TCCACAATCC ATGAGGAAAC TTCGTATTCT TGCGATCGTT CTCATAGTTT |
| 701 | TTAGCATTAT TTTGATTGCA AGTGGTGTGG TATTGCTTAC TGTAGCGATC |
| 751 | CCTGGATTAA GCTCGATCAT TTCTTCCCCA GCGGAGATGG GPGCTTGTGC |
| 801 | TTTGGGATGT GTGATGCTTG CTTTGGGGAT CGACGTTCTT CTGAAGAAAC |
| 851 | GAGAAGTCCC TATAGTAGTT CCCGCACCTA TTCCTGAAGA AGTCGTCATA |
| 901 | GATGATATAG ATGAAGAGAG TATACGGCTG CAGCAGGAAG CTGAAGCCGC |
| 951 | TTTAGCAAGA CTTCCTGAGG AGATGAGTGC ATTTGAAGGT TACATAAAAG |
| 1001 | TTGTCGAGAG TCATTTGGAG AACATGAAAA GCCTGCCTTA TGATGGTCAT |
| 1051 | GGGCTAGAAG AGAAAACGAA ACATCAGATA AGAGTCGTCA GATCTTCTTT |
| 1101 | GAAGGCTATG GTTCCAGAAT TTTTAGATAT CAGAAGAATT TTTGAAGAAG |
| 1151 | AAGAGTTCTT TTTTCTCTCA GCTCGCAAAC GACTTATAGA TTTAGCTACT |
| 1201 | ACTTTAGTAG AGAGAAAAAT TTAAACAGAG CAACTTGAGC GCAATAATTT |
| 1251 | AAGGAAAGCG TTTTCTTATT TATATCAGGA CTCAATTTTT AAAAAAATTA |
| 1301 | TTGATAACTT CGAGAAGTTA GCATGGAAAT TTATGATTTT GAGTAAATCA |
| 1351 | ATTTGTCGAT TTACAATTAT TTTTGAAAAT CATGAACATG GTGTAGCAAA |
| 1401 | GAGCCTGTTA CACAAGAATG CAGTGTTACT GGAGAAGGTA ATCTATAGGA |
| 1451 | GTTTGCAAAA AAGCTATAGA GATATAGGCA TGTCATCTGC AAAGATGAAA |
| 1501 | ATCTTGCACG GCAACCCTTT TTTCTCTTTG GAAGATAATA AAAAGACGAT |
| 1551 | AATGAAAGAA CACGCAGAGA TGCTTGAAAG TCTCAGTAGC TATAGGAAGG |
| 1601 | TATTTTTAGC TCTATCTGAT GAGAACGTTG TAGATACACC TAGCGATCCA |
| 1651 | AAGAAATGGG ATTTGTCAGG AATCCCCTGT AGGGACGCGT TGTCTGAGAT |
| 1701 | TTCTCGTGAT GAACAGTGGC AGAAGAAAGC ACATCTAAAG CATCAAGAGT |
| 1751 | CCCTCTATAC GCAAGCTAGG GATCGTTTAA CAGACCAGAG CTCTAAAGAA |
| 1801 | AATCAGAAAG AGTTAGAGAA AGCTGAACAA GAGTACATAT CTTCTTGGGA |
| 1851 | ACGGGTTAAA AAATTTGAGA TTGAGAGAGT ACAGGAGAGG ATACGGGCAA |
| 1901 | TTCAAAAGCT TTATCCTAAT ATCCTCGAGA GAGAAGAAGA AACCACAGGT |
| 1951 | CAGGAGACTG TGACTCCAAC TGTTCAAGGG ACGACGGCTT CATCCGATTT |
| 2001 | AACAGATATT TTAGGAAGAA TAGAGGTCTC CAGTAGGGAG GATAATCAGA |
| 2051 | ATCAAGAGTC TTGTGTAAAA GTCTTAAGAA GTCATGAGGT AGAAATGAGC |
| 2101 | TGGGAAGTCA AACAAGAGTA TGGCCCTAAG AAAAAAGAAT TTCAGGATCA |
| 2151 | AATGGGTTCT TTAGAGAGGT TTTTTACAGA GCATATTGAA GAGTTAGAAG |
| 2201 | TATTACAGAA GGACTACTCT AAACACTTGT CTTATTTTAA AAAAGTAAAC |
| 2251 | AATAAGAAAG AGGTTCAATA TGCGAAGTTT AGGTTGAAGG TTTTAGAGTC |
| 2301 | AGATTTAGAA GGGATTCTAG CTCAGACTGA GAGTGCTGAG AGTCTGTTAA |
| 2351 | CTCAAGAAGA ACTTCCGATT CTTGCAACTC GGGGAGCCTT AGAGAAAGCT |
| 2401 | GTTTTCAAAG GGAGTCTATG TTGCGCGCTA GCAAGCAAAG CAAAACCCTA |
| 2451 | TTTTGAAGAG GATCCCAGAT TCCAAGATTC TGATACGCAA TTGCGAGCTC |
| 2501 | TGACTCTAAG GTTACAGGAG GCTAAGGCAA GCCTGGAAGA AGAGATAAAG |
| 2551 | AGATTTTCAA ATCTTGAGAA CGATATTGCA GAGGAAAGAC GCCTTCTTAA |
| 2601 | AGAGAGCAAG CAGACGTTCG AAAGAGCAGG TTTAGGGGTT CTCCGAGAAA |
| 2651 | TTGCAGTCGA GTCTACTTAT GATTTGCGTT CCTTAACAAA TACATGGGAA |
| 2701 | GCGACCCCAG AGAGTGAGAA GGTCTATTTT AGCATGTATC TTAATTATTA |
| 2751 | CAACGAAGAG AAACGTAGGG CTAAAACAAG ATTGGTTGAA ATGACACAGA |
| 2801 | GGTATAGAGA TTTTAAAATG GCCTTGGAAG CTATGCAGTT TAATGAAGAA |
| 2851 | GCCCTTTTGC AAGAGGAACT CTCTATTCAA GCTCCCAGTG AATAA |

The PSORT algorithm predicts inner membrane (0.660).

The protein was expressed in E. coli and purified as a GST-fusion product (FIG. 68A). The recombinant protein was used to immunise mice, whose sera were used in a Western blot (FIG. 68B) and for FACS analysis.

These experiments show that cp6262 is a surface-exposed and immunoaccessible protein, and that it is a useful immunogen. These properties are not evident from the sequence alone.

### Example 69

The following C. pneumoniae protein (PID 4376269) was expressed <SEQ ID 137, cp6269>:

| | |
|---|---|
| 1 | MYQENLRLLE RLLYNSVQKS YADRLFSYEK TKMVHDTPLI PWEEDKEKCA |
| 51 | EAEKAFLEQQ KILLDYGKSI FWLNENDEIN LNDPWSWGLN TVRTRKVFQE |
| 101 | VDDSERWNHK VLIQKLEDDY EKLLEESSKE STEANKKLLS DLVDRLEDAK |
| 151 | TKFFLKKQEE VETRVKDLRA RYGGTVDPKQ DTEAKKKVEL EASLETFLDS |
| 201 | IESELVQCLE DQDIYWKEQD VKDLARTQEL EEQDIEAKRE EAAEDLRSLN |
| 251 | ERLKKSKTML DRAKWHIENA EDSITWWTSQ IEMKDMKARL KILKBDITSV |
| 301 | LPEIDEIETC LSLEELPLLT TRELLTKSYL KFKICSETLL KMTSVFENNI |
| 351 | YVQEYEVQLQ NLGFKLQGIS QRFGKKQDDF ANLEEQVALQ KKRLRELTQN |
| 401 | FEIQGFNFMK EDFKAAAKDL YIRSTAEQKM NFDVPCMELF RRYHEEVNKP |
| 451 | LLELMYNCAD SYRDAKKKLC SLRLDEKELL QKEIKKEEFY QKKQQRHADR |
| 501 | SRHTTYQKLR IAEELALELK KKI* |

The cp6269 nucleotide sequence <SEQ ID 138> is:

| | |
|---|---|
| 1 | ATGTACCAGG AGAATCTAAG ATTGTTGGAA AGGCTTCTTT ATAATAGTGT |
| 51 | TCAAAAGAGC TATGCGGATC GGCTGTTTC CTATGAAAAG ACAAAGATGG |
| 101 | TGCACGATAC TCCGCTGATT CCTTGGGAAG AGGATAAGGA AAAATGTGCT |
| 151 | GAAGCTGAGA AAGCTTTCTT AGAGCAACAG AAGATTCTCC TAGATTATGG |
| 201 | AAAATCTATC TTTTGGCTGA ATGAGAACGA TGAGATCAAT TTAAACGATC |
| 251 | CTTGGAGTTG GGGTCTTAAT ACGGTGAGGA CTAGGAAAGT ATTCCAAGAG |
| 301 | GTTGACGACA CTGAACGTTG GAATCATAAG GTACTCATTC AAAAACTCGA |
| 351 | GGACGATTAT GAGAAACTTC TAGAGGAAAG TTCAAAAGAG TCTACTGAAG |
| 401 | CAAATAAGAA GCTTTTATCT GACTTAGTAG ATCGTCTTGA AGATGCTAAG |
| 451 | ACAAAATTTT TCCTGAAGAA ACAGGAGGAG GTGGAGACTC GCGTTAAGGA |
| 501 | TCTTAGAGCT CGATATGGAG GCACAGTAGA TCCTAAGCAG GATACGGAAG |
| 551 | CTAAGAAGAA AGTCGAATTG GAGGCTAGCT TAGAAACCTT TTTAGATTCC |
| 601 | ATCGAATCAG AGCTAGTACA GTGTTTAGAA GATCAAGATA TATATTGGAA |
| 651 | AGAACAGGAT GTCAAAGATC TAGCACGTAC GCAAGAGCTC GAGGAACAAG |
| 701 | ATATTGAAGC GAAGAGGGAA GAAGCTGCCG AAGACCTAAG AAGTCTTAAT |
| 751 | GAGCGTTTAA AGAAGTCAAA AACTATGTTA GATAGGGCTA AATGGCATAT |
| 801 | TGAAAATGCT GAGGACAGTA TTACCTGGTG GACTAGTCAG ATAGAAATGA |
| 851 | AGGATATGAA AGCAAGACTG AAGATCTTAA AAGAAGATAT AACAAGTGTT |
| 901 | CTACCTGAAA TAGATOAGAT TGAAACGTGT TTAAGCTTAG AGGAGCTTCC |
| 951 | TTTGCTTACG ACCAGGGAAC TCTTAACTAA GTCCTACCTA AAGTTTAAGA |
| 1001 | TTTGTTCGGA AACACTATTA AAAATGACTT CTGTGTTTGA GAACAATATC |
| 1051 | TATGTTCAGG AGTACGAGGT TCAGCTGCAA AATCTAGGGT TTAAGTTACA |
| 1101 | AGGTATATCT CAGAGATTCG GAAAGAAACA AGACGATTTT GCGAATCTAG |
| 1151 | AGGAACAGGT TGCTTTGCAA AAGAAACGAC TCAGAGAGCT CACTCAGAAT |
| 1201 | TTTGAAATAC AAGGATTCAA TTTCATGAAA GAAGATTTTA AGGCAGCCGC |
| 1251 | TAAAGATCTT TATATAAGAA GTACAGCTGA ACAAAAGATG AACTTTGATG |
| 1301 | TGCCTTGCAT GGAGCTCTTC CGTAGGTATC ATGAGGAGGT CAACAAGCCG |
| 1351 | CTTCTTGAGT TGATGTACAA TTGTGCAGAC AGTTATAGAG ATGCTAAGAA |
| 1401 | AAAGCTTTGC TCTCTACGTC TTGATGAAAA AGAGTTATTA CAAAAAGAAA |
| 1451 | TCAAGAAAGA GGAATTTTAT CAAAAGAAAC AACAAAGGCA TGCAGATAGA |
| 1501 | TCACGTCATA CTACGTATCA AAAGCTACGA ATTGCTGAAG AGCTTGCTCT |
| 1551 | TGAGCTGAAG AAGAAAATCT AA |

The PSORT algorithm predicts cytoplasmic location (0.412).

The protein was expressed in E. coli and purified as a GST-fusion product (FIG. 69A). The recombinant protein was used to immunise mice, whose sera were used in a Western blot (FIG. 69B) and for FACS analysis.

These experiments show that cp6269 is a surface-exposed and immunoaccessible protein, and that it is a useful immunogen. These properties are not evident from the sequence alone.

### Example 70

The following C. pneumoniae protein (PID 4376270) was expressed <SEQ ID 139; cp6270>:

| | |
|---|---|
| 1 | MKIPLRFLLI SLVPTLSMSN LLGAATTEEL SASNSFDGTT STTSFSSKTS |
| 51 | SATDGTNYVF KDSVVIENVP KTGETQSTSC FKNDAAAGDL NFLGGGFSFT |
| 101 | FSNIDATTAS GAAIGSEAAN KTVTLSGFSA LSFLKSPAST VTNGLGAINV |
| 151 | KGNLSLLDND KVLIQDNFST GDGGAINCAG SLKIANNKSL SFIGNSSSTR |
| 201 | GGAIHTKNLT LSSGGETLFQ GNTAPTAAGK GGAIAIADSG TLSISGDSGD |
| 251 | IIFEGNTIGA TGTVSHSAID LGTSAKITAL RAAQGHTIYF YDPITVTGST |
| 301 | SVADALNINS PDTGDNKEYT GTIVFSGEKL TEAEAKDEKN RTSKLLQNVA |
| 351 | FKNGTVVLKG DVVLSANGFS QDANSKLIMD LGTSLVANTE SIELTNLEIN |
| 401 | IDSLRNGKKI KLSAATAQKD IRIDRPVVLA ISDESFYQNG FLNEDHSYDG |
| 451 | ILELDAGKDI VISADSRSID AVQSPYGYQG KWTINWSTDD KKATVSWAKQ |
| 501 | SFNPTAEQEA PLVPNLLWGS FIDVRSFQNF IELGTEGAPY EKRFWVAGIS |
| 551 | NVLHRSGREN QRKFRHVSGG AVVGASTRMP GGDTLSLGFA QLFARDKDYF |
| 601 | MNTNFAKTYA GSLRLQHDAS LYSVVSILLG EGGLREILLP YVSKTLPCSF |
| 651 | YGQLSYGHTD HRMKTESLPP PPPTLSTDHT SWGGYVWAGE LGTRVAVENT |
| 701 | SGRGFFQEYT PFVKVQAVYA RQDSFVELGA ISRDFSDSHL YNLAIPLGIK |
| 751 | LEKRFAEQYY HVVAMYSPDV CRSNPKCTTT LLSNQGSWKT KGSNLARQAG |
| 801 | IVQASGFRSL GAAAELFGNF GFEWRGSSRS YNVDAGSKIK F |

A predicted signal peptide is highlighted.

The cp6270 nucleotide sequence <SEQ ID 140> is:

| | |
|---|---|
| 1 | ATGAAGATTC CACTCCGCTT TTTATTGATA TCATTAGTAC CTACGCTTTC |
| 51 | TATGTCGAAT TTATTAGGAG CTGCTACTAC CGAAGAGTTA TCGGCTAGCA |
| 101 | ATAGCTTCGA TGGAACTACA TCAACAACAA GCTTTTCTAG TAAAACATCA |
| 151 | TCGGCThCAG ATGGCACCAA TTATGTTTTT AAAGATTCTG TAGTTATAGA |
| 201 | AAATGTACCC AAAACAGGGG AAACTCAGTC TACTAGTTGT TTTAAAAATG |
| 251 | ACGCTGCAGC TGGAGATCTA AATTTCTTAG GAGGGGGATT TTCTTTCACA |
| 301 | TTTAGCAATA TCGATGCAAC CACGGCTTCT GGAGCTGCTA TTGGAAGTGA |
| 351 | AGCAGCTAAT AAGACAGTCA CGTTATCAGG ATTTTCGGCA CTTTCTTTTC |
| 401 | TTAAATCCCC AGCAAGTACA GTGACTAATG GATTGGGAGC TATCAATGTT |
| 451 | AAAGGGAATT TAAGCCTATT GGATAATGAT AAGGTATTGA TTCAGGACAA |
| 501 | TTTCTCAACA GGAGATGGCG GAGCAATTAA TTGTGCAGGC TCCTTGAAGA |
| 551 | TCGCAAACAA TAAGTCCCTT TCTTTTATTG GAAATAGTTC TTCAACACGT |
| 601 | GGCGGAGCGA TTCATACCAA AAACCTCACA CTATCTTCTG GTGGGGAAAC |
| 651 | TCTATTTCAG GGGAATACAG CGCCTACGGC TGCTGGTAAA GGAGGTGCTA |
| 701 | TCGCGATTGC AGACTCTGGC ACCCTATCCA TTTCTGGAGA CAGTGGCGAC |
| 751 | ATTATCTTTG AAGGCAATAC GATAGGAGCT ACAGGAACCG TCTCTCATAG |
| 801 | TGCTATTGAT TTAGGAACTA GCGCTAAGAT AACTGCGTTA CGTGCTGCGC |
| 851 | AAGGACATAC GATATACTTT TATGATCCGA TTACTGTAAC AGGATCGACA |
| 901 | TCTGTTGCTG ATGCTCTCAA TATTAATAGC CCTGATACTG GAGATAACAA |
| 951 | AGAGTATACG GGAACCATAG TCTTTTCTGG AGAGAAGCTC ACGGAGGCAG |
| 1001 | AAGCTAAAGA TGAGAAGAAC CGCACTTCTA AATTACTTCA AAATGTTGCT |
| 1051 | TTTAAAAATG GGACTGTAGT TTTAAAAGGT GATGTCCTTT TAAGTGCGAA |
| 1101 | CGGTTTCTCT CAGGATGCAA ACTCTAAGTT GATTATGGAT TTAGGGACGT |
| 1151 | CGTTGGTTGC AAACACCGAA AGTATCGAGT TAACGAATTT GGAAATTAAT |
| 1201 | ATAGACTCTC TCAGGAACGG GAAAAAGATA AAACTCAGTG CTGCCACAGC |
| 1251 | TCAGAAAGAT ATTCGTATAG ATCGTCCTGT TGTACTGGCA ATTAGCGATG |
| 1301 | AGAGTTTTTA TCAAAATGGC TTTTTGAATG AGGACCATTC CTATGATGGG |
| 1351 | ATTCTTGAGT TAGATGCTGG GAAAGACATC GTGATTTCTG CAGATTCTCG |
| 1401 | CAGTATAGAT GCTGTACAAT CTCCGTATGG CTATCAGGGA AAGTGGACGA |
| 1451 | TCAATTGGTC TACTGATGAT AAGAAAGCTA CGGTTTCTTG GGCGAAGCAG |
| 1501 | AGTTTTAATC CCACTGCTGA GCAGGAGGCT CCGTTAGTTC CTAATCTTCT |
| 1551 | TTGGGGTTCT TTTATAGATG TTCGTTCCTT CCAGAATTTT ATAGAGCTAG |
| 1601 | GTACTGAAGG TGCTCCTTAC GAAAAGAGAT TTTGGGTTGC AGGCATTTCC |
| 1651 | AATGTTTTGC ATAGGAGCGG TCGTGAAAAT CAAAGGAAAT TCCGTCATGT |
| 1701 | GAGTGGAGGT GCTGTAGTAG GTCCTAGCAC GAGGATGCCG GGTGGTGATA |
| 1751 | CCTTGTCTCT GGGTTTTGCT CAGCTCTTTG CGCGTGACAA AGACTACTTT |
| 1801 | ATGAATACCA ATTTCGCAAA GACCTACGCA GGATCTTTAC CTTTGCAGCA |
| 1851 | CGATGCTTCC CTATACTCTG TGGTGAGTAT CCTTTTAGGA GAGGGAGGAC |
| 1901 | TCCGCGAGAT CCTGTTGCCT TATGTTTCCA AGACTCTGCC GTGCTCTTTC |
| 1951 | TATGGGCAGC TTAGCTACGG CCATACGGAT CATCGCATGA AGACCGAGTC |
| 2001 | TCTACCCCCC CCCCCCCCGA CGCTCTCGAC GGATCATACT TCTTGGGGAG |
| 2051 | GATATGTCTG GGCTGGAGAG CTGGGAACTC GAGTTGCTGT TGAAAATACC |
| 2101 | AGCGGCAGAG GATTTTTCCA AGAGTACACT CCATTTGTAA AAGTCCAAGC |
| 2151 | TGTTTACGCT CGCCAAGATA GCTTTGTAGA ACTAGGAGCT ATCAGTCGTG |
| 2201 | ATTTTAGTGA TTCGCATCTT TATAACCTTG CGATTCCTCT TGGAATCAAG |
| 2251 | TTAGAGAAAC GGTTTGCAGA GCAATATTAT CATGTTGTAG CGATGTATTC |
| 2301 | TCCAGATGTT TGTCGTAGTA ACCCCAAATG TACGACTACC CTACTTTCCA |
| 2351 | ACCAAGGGAG TTGGAAGACC AAAGGTTCGA ACTTAGCAAG ACAGGCTGGT |
| 2401 | ATTGTTCAGG CCTCAGGTTT TCGATCTTTG GGAGCTGCAG CAGAGCTTTT |
| 2451 | CGGGAACTTT GGCTTTGAAT GGCGGGGAAT GGCGGGGATC TTCTCGTAGC |
| 2501 | ATGCGGGTAG CAAAATCAAA TTTTAG |

The PSORT algorithm predicts outer membrane (0.92).

The protein was expressed in E. coli and purified as a GST-fusion product (FIG. 70A). The recombinant protein was used to immunise mice, whose sera were used in a Western blot and for FACS analysis (FIG. 70B).

The cp6270 protein was also identified in the 2D-PAGE experiment (Cpn0013).

These experiments show that cp6270 is a surface-exposed and immunoaccessible protein, and that it is a useful immunogen. These properties are not evident from the sequence alone.

### Example 71

The following C. pneumoniae protein (PID 4376402) was expressed <SEQ ID 141; cp6402>:

| | |
|---|---|
| 1 | MNVADLLSHL ETLLSSKIFQ DYGPNGLQVG DPQTPVKKIA VAVTADLETI |
| 51 | KQAVAAEANV LIVHHGIFWK GMPYPITGMI HKRIQLLIEH NIQLIAYHLP |
| 101 | LDAHPTLGNN WRVALDLNWH DLKPFGSSLP YLGVQGSFSP IDIDSFIDLL |
| 151 | SQYYQAPLKG SALGGPSRVS SAALISGGAY RELSSAATSQ VDCFITGNFD |
| 201 | EPAWSTALES NINFLAFGHT ATEKVGPKSL AEHLKSEFPI STTFIDTANP |
| 251 | F |

The cp6402 nucleotide sequence <SEQ ID 142> is:

| | |
|---|---|
| 1 | ATGAATGTTG CGGAPCTCCT TTCTCATCTT GAGACTCTTC TCTCATCAAA |
| 51 | AATATTTCAG GATTATGGAC CCAACGGACT TCAAGTTGGA GATCCCCAAA |
| 101 | CTCCGGTAAA GAAAATCGCT GTTGCAGTTA CCGCAGATCT AGAAACCATA |
| 151 | AAACAAGCTG TTGCGGCCGA AGCAAACGTT CTCATTGTAC ACCACGGAAT |
| 201 | TTTTTGGAAA GGTATGCCCT ATCCTATTAC CGGCATGATC CATAAGCGCA |
| 251 | TCCAATTACT AATAGAACAC AATATCCAAC TCATTGCCTA CCACCTTCCT |
| 301 | TTGGATGCTC ACCCTACCTT AGGAAATAAC TGGAGAGTTG CCCTGGATCT |
| 351 | AAATTGGCAT GACTTGAAGC CCTTTGGTTC TTCCCTCCCT TATTTAGGAG |
| 401 | TGCAAGGCTC TTTCTCTCCT ATCGATATAG ATTCTTTCAT TGACCTGTTA |
| 451 | TCTCAATATT ACCAAGCTCC CCTAAAAGGA TCTGCCTTGG GCGGCCCCTC |
| 501 | TAGAGTCTCC TCAGCAGCTC TGATCTCAGG AGGAGCTTAT AGAGAACTCT |
| 551 | CTTCGGCAGC CACGTCCCAA GTCGATTGCT TCATCACAGG AAATTTTGAT |
| 601 | GAACCTGCAT GGTCGACAGC TCTAGAAAGC AATATCAACT TCCTAGCATT |
| 651 | TGGACATACA GCCACAGAAA AAGTAGGTCC AAAATCTCTT GCAGAGCATC |
| 701 | TAAAAAGCGA ATTTCCTATT TCCACAACCT TTATAGATAC GGCCAACCCC |
| 751 | TTCTAA |

The PSORT algorithm predicts cytoplasmic (0.158).

The protein was expressed in E. coli and purified as a GST-fusion product (FIG. 71A). The recombinant protein was used to immunise mice, whose sera were used in a Western blot (FIG. 71B) and for FACS analysis.

These experiments show that cp6402 is a surface-exposed and immunoaccessible protein, and that it is a useful immunogen. These properties are not evident from the sequence alone.

### Example 72

The following C. pneumoniae protein (PID 4376520) was expressed <SEQ ID 143; cp6520>:

| | |
|---|---|
| 1 | MKHYLSFSPS ADFFSKQGAI ETQVLFGERV LVKGSTCYAY SQLFHNELLW |
| 51 | KPYPGHSFRS TLVPCTPEFH IHPNVSVVSV DAFLDPWGIP LPFGTLLHVN |
| 101 | SQNTVIFPKD ILNHMNTIWG SGTPQCDPRH LRRLNYNFFA ELLIKDADLL |
| 151 | LNFPYVWGGR SVHESLEKPG VDCSGFINIL YQAQGYNVPR NAADQYADCH |
| 201 | WISSFENLPS GGLIFLYPKE EKRISHVMLK QDSSTLIHAS GGGKKVEYFI |
| 251 | LEQDGKFLDS TYLFFRNNQR GRAFFGIPRK RKAPL* |

The cp6520 nucleotide sequence <SEQ ID 144> is:

| | |
|---|---|
| 1 | ATGAAACAGT ACCTATCATT TTCTCCTTCT GCTGATTTTT TCTCTAAACA |
| 51 | GGGTGCTATT GAAACTCAAG TCCTTTTTGG AGAGCGCGTC TTAGTCAAAG |
| 101 | GGAGCACCTG CTATGCATAT TCCCAATTAT TCCACAATGA GCTGTTATGG |
| 151 | AAGCCCTATC CAGGTCATAG CTTTCGTTCT ACCCTAGTCC CCTGCACTCC |
| 201 | TGAATTTCAT ATCCATCCAA ATGTTTCTGT GGTTTCTGTG GATGCATTTT |
| 251 | TAGATCCTTG GGGGATCCCT CTTCCTTTTG GAACTTTACT CCATGTGAAT |
| 301 | TCTCAAAATA CCGTTATTTT CCCTAAGGAT ATTCTCAATC ATATGAACAC |
| 351 | CATCTGGGGC TCCGGCACAC CTCAATGCGA TCCTAGACAT CTACGTCGTC |
| 401 | TAAATTATTA CTTCTTTGCT GAACTTTTAA TTAAAGACGC AGACCTTTTA |
| 451 | CTGAACTTTC CCTATGTATG GGGAGGACGG TCTGTACACG AAAGTCTGGA |
| 501 | AAAGCCGGGT GTTGATTGTT CGGGATTTAT CAATATCCTT TACCAGGCAC |
| 551 | AGGGATACAA CGTCCCTAGA AACGCTGCAG ATCAATATGC GGATTGTCAT |
| 601 | TGGATCTCTA GCTTTGAGAA CCTTCCTTCT GGTGGGTTAA TATTTCTTTA |
| 651 | CCCTAAAGAA GAAAAGCGTA TTTCTCATGT TATGTTGAAA CAGGATAGTT |
| 701 | CCACCCTCAT TCATGCTTCT GGTGGAGGGA AAAAAGTGGA GTATTTCATT |
| 751 | TTAGAACAAG ATGGGAAGTT TTTAGATTCG ACTTATCTAT TTTTTAGAAA |
| 801 | TAATCAGAGG GGACGGGCAT TTTTTGGGAT CCCTAGAAAA AGAAAAGCCT |
| 851 | TTCTGTAA |

The PSORT algorithm predicts cytoplasmic (0.265).

The protein was expressed in E. coli and purified as a GST-fusion product (FIG. 72A). The recombinant protein was used to immunise mice, whose sera were used in a Western blot (FIG. 72B) and for FACS analysis.

These experiments show that cp6520 is a surface-exposed and immunoaccessible protein, and that it is a useful immunogen. These properties are not evident from the sequence alone.

### Example 73

The following C. pneumoniae protein (PID 4376567) was expressed <SEQ ID 145; cp6567>:

| | |
|---|---|
| 1 | MTSPIPFQSS GDASFLAEQP QQLPSTSESQ LVTQLLTMMK HTQALSETVL |
| 51 | QQQRDRLPTA SIILQVGGAP TGGAGAPFQP GPADDHHHPI PPPVVPAQIE |
| 101 | TEITTIRSEL QLMRSTLQQS TKGARTGVLV VTAILMTISL LAIIIIILAV |
| 151 | LGFTGVLPQV ALLMQGETNL IWAMVSGSII CFIALIGTLG LILTNKNTPL |

The cp6567 nucleotide sequence <SEQ ID 146> is:

| | |
|---|---|
| 1 | ATGACCTCAC CGATCCCCTT TCAGTCTAGT GGCGATGCCT CTTTCCTTGC |
| 51 | CGAGCAGCCA CAGCAACTCC CGTCTACTTC TGAATCTCAG CTAGTAACTC |
| 101 | AATTGCTAAC CATGATGAAG CATACTCAAG CATTATCCGA AACGGTTCTT |
| 151 | CAACAACAAC GCGATCGATT ACCAACCGCA TCTATTATCC TTCAAGTAGG |
| 201 | AGGAGCTCCT ACAGGAGGAG CGGGTGCGCC TTTTCAACCA GGACCGGCAG |
| 251 | ATGATCATCA TCATCCCATA CCGCCGCCTG TTGTACCAGC TCAAATAGAA |
| 301 | ACAGAAATCA CCACTATAAG ATCCGAGTTA CAGCTCATGC GATCTACTCT |
| 351 | ACAACAAAGC ACAAAAGGAG CTCGTACAGG AGTTCTAGTG GTTACTGCAA |
| 401 | TCTTAATGAC GATCTCCTTA TTGGCTATTA TTATCATAAT ACTAGCTGTG |
| 451 | CTTGGATTTA CGGGCGTCTT GCCTCAAGTA GCTTTATTGA TGCAGGGTGA |
| 501 | AACAAATCTG ATTTGGGCTA TGGTGAGCGG TTCTATTATT TGCTTTATTG |
| 551 | CGCTAATTGG AACTCTAGGA TTAATTTTAA CAAATAAGAA CACGCCTCTA |
| 601 | CCGGCTTCTT AA |

The PSORT algorithm predicts inner membrane (0.694).

The protein was expressed in E. coli and purified as a GST-fusion product (FIG. 73A). The recombinant protein was used to immunise mice, whose sera were used in a Western blot (FIG. 73B) and for FACS analysis.

These experiments show that cp6567 is a surface-exposed and immunoaccessible protein, and that it is a useful immunogen. These properties are not evident from the sequence alone.

### Example 74

The following C. pneumoniae protein (PID 4376576) was expressed <SEQ ID 147; cp6576>:

| | |
|---|---|
| 1 | MLIMRNKVIL QISILALIQT PLTLFSTEKV KEGHVVVDSI TIITEGENAS |
| 51 | NKHPLPKLKT RSGALFSQLD FDEDLRILAK EYDSVEPKVE FSEGKTNIAL |
| 101 | HLIAKPSIRN IHISGNQVVP EHKILKTLQI YRNDLFEREK FLKGLDDLRT |
| 151 | YYLKRGYFAS SVDYSLEHNQ EKGHIDVLIK INEGPCGKIK QLTFSGISRS |
| 201 | EKSDIQEFIQ TKQHSTTTSW FTGAGLYHPD IVEQDSLAIT NYLHNNGYAK |
| 251 | AIVNSHYDLD DKGNILLYKD IDRGSRYTLG HVHIQGFEVL PKRLIEKQSQ |
| 301 | VGPNDLYCPD KIWDGAHKIK QTYAKYGYIN TNVDVLFIPH ATRPIYDVTY |
| 351 | EVSEGSPYKV GLIKITGNTH TKSDVILHET SLFPGDTFNR LKLEDTEQRL |
| 401 | RNTGYFQSVS VYTVRSQLDP MGNADQYRDI FVEVKETTTG NLGLFLGFSS |
| 451 | LDNLFGGIEL SESNFDLFGA RNIFSKGFRC LRGGGEHLFL KANFGDKVTD |
| 501 | YTLKWTKPHF LNTPWILGIE LDKSINRALS KDYAVQTYGG NVSTTYILNE |
| 551 | HLKYGLFYRG SQTSLHEKRK FLLGPNIDSN KGFVSAAGVN LNYDSVDSPR |
| 601 | TPTTGIRGGV TFEVSGLGGT YHFTKLSLNS SIYRKLTRKG ILKIKGEAQF |
| 651 | IKPYSNTTAE GVPVSERFFL GGETTVRGYK SFIIGPKYSA TEPQGGLSSL |
| 701 | LISEEFQYPL IRQPNISAFV FLDSGFVGLQ EYKISLKDLR SSAGFGLRFD |
| 751 | VMNNVPVMLG FGWPFRPTET LNGEKIDVSQ RFFFALGGMF * |

A predicted signal peptide is highlighted.

The cp6576 nucleotide sequence <SEQ ID 148> is:

| | |
|---|---|
| 1 | ATGCTCATCA TGCGAAATAA AGTTATCTTG CAAATATCTA TTCTAGCGTT |
| 51 | AATCCAAACC CCTTTAACTT TATTTTCTAC TGAAAAAGTT AAAGAAGGCC |
| 101 | ATGTGGTGGT AGACTCTATC ACAATCATAA CGGAAGGAGA AAATGCTTCA |
| 151 | AATAAACATC CCTTACCCAA ATTAAAGACC AGAAGTGGGG CTCTTTTTTC |
| 201 | TCAATTAGAT TTTGATGAAG ACTTGAGAAT TCTAGCTAAA GAATACGACT |
| 251 | CTGTTGAGCC TAAAGTAGAA TTTTCTGAAG GGAAAACTAA CATAGCCCTT |
| 301 | CACCTAATAG CTAAACCCTC AATTCGAAAT ATTCATATCT CAGGAAATCA |
| 351 | AGTCGTTCCT GAACATAAAA TTCTTAAAAC CCTACAAATT TACCGTAATG |
| 401 | ATCTCTTTGA ACGAGAAAAA TTTCTTAAGG GTCTTGATGA TCTAAGAACG |
| 451 | TATTATCTCA AGCGAGGATA TTTCGCATCC AGTGTAGACT ACAGTCTGGA |
| 501 | ACACAATCAA GAAAAAGGTC ACATCGATGT TTTAATTAAA ATCAATGAAG |
| 551 | GTCCTTGCGG GAAAATTAAA CAGCTTACGT TCTCAGGAAT CTCTCGATCA |
| 601 | GAAAAATCAG ATATCCAAGA ATTTATTCAA ACCAAGCAGC ACTCTACAAC |
| 651 | TACAAGTTGG TTTACTGGAG CTGGACTCTA TCACCCAGAT ATTGTTGAAC |
| 701 | AAGATAGCTT GGCAATTACG AATTACCTAC ATAATAACGG GTACGCTGAT |
| 751 | GCTATAGTCA ACTCTCACTA TGACCTTGAC GACAAAGGGA ATATTCTTCT |
| 801 | TTACATGGAT ATPGATCGAG GGTCGCGATA TACCTTAGGA CACGTCCATA |
| 851 | TCCAAGGGTT TGAGGTTTTG CCAAAACGCC TTATAGAAAA GCAATCCCAA |
| 901 | GTCGGCCCCA ATGATCTTTA TTGCCCCGAT AAAATATGGG APGGGGCTCA |
| 951 | TAAGATCAAA CAAACTTATG CAAAGTATGG CTACATCAAT ACCAATGTAG |
| 1001 | ACGTTCTCTT CATCCCTCAC GCAACCCGCC CTATTTATGA TGTAACTTAT |
| 1051 | GAGGTAAGTG AAGGGTCTCC TTATAAAGTT GGGTTAATTA AAATTACTGG |
| 1101 | GAATACCCAT ACAAAATCTG ACGTTATTTT ACACGAAACC AGTCTCTTCC |
| 1151 | CAGGAGATAC ATTCAATCGC TTAAAGCTAG AAGATACTGA GCAACGTTTA |
| 1201 | AGAAATACAG GCTACTTCCA AAGCGTTAGT GTCTATACAG TTCGTTCTCA |
| 1251 | ACTTGATCCT ATGGGCAATG CGGATCAATA CCGAGATATT TTTGTAGAAG |
| 1301 | TCAAAGAAAC AACAACAGGA AACTTAGGCT TATTCTTAGG ATTTAGTTCT |
| 1351 | CTTGACAATC TTTTTGGAGG AATTGAACTA TCTGAAAGTA ATTTTGATCT |
| 1401 | ATTTGGAGCT AGAAATATAT TTTCTAAAGG TTTTCGTTGT CTAAGAGGCG |
| 1451 | GTGGAGAACA TCTATTCTTA AAAGCCAACT TCGGGGACAA AGTCACAGAC |
| 1501 | TATACTTTGA AGTGGACCAA ACCTCATTTT CTAAACACTC CTTGGATTTT |
| 1551 | AGGAATTGAA TTAGATAAAT CAATTAACAG AGCATTATCT AAAGATTATG |
| 1601 | CTGTCCAAAC CTATGGCGGG AACGTCAGCA CAACGTATAT CTTGAACGAA |
| 1651 | CACCTGAAAT ACGGTCTATT TTATCGAGGA AGTCAAACGA GTTTACATGA |
| 1701 | AAAACGTAAG TTCCTCCTAG GGCCAAATAT AGACAGCAAT AAAGGATTTG |
| 1751 | TCTCTGCTGC AGGTGTCAAC TTGAATTACG ATTCTGTAGA TAGTCCTAGA |
| 1801 | ACTCCAACTA CAGGGATTCG CGGGGGGGTG ACTTTTGAGG TTTCTGGTTT |
| 1851 | GGGAGGAACT TATCATTTTA CAAAACTCTC TTTAAACAGC TCTATCTATA |
| 1901 | GAAAACTTAC GCGTAAAGGT ATTTTGAAAA TCAAAGGCGA AGCTCAATTT |
| 1951 | ATTAAACCCT ATAGCAATAC TACAGCTGAA GGAGTTCCTG TCAGTGAGCG |
| 2001 | CTTCTTCCTA GGTGGAGAGA CTACAGTTCG GGGATATAAA TCCTTTATTA |
| 2051 | TCGGTCCAAA ATACTCTGCT ACAGAACCTC AGGGAGGACT CTCTTCGCTC |
| 2101 | CTTATTTCAG AAGAGTTTCA ATACCCTCTC ATCAGACAAC CTAATATTAG |
| 2151 | TGCCTTTGTA TTCTTAGACT CAGGTTTTGT CGGTTTACAA GAGTATAAGA |
| 2201 | TTTCGTTAAA AGATCTACGT AGTAGTGCTG GATTTGGTCT GCGCTTCGAT |
| 2251 | GTAATGAATA ATGTTCCTGT TATGTTAGGA TTTGGTTGGC CCTTCCGTCC |
| 2301 | AACCGAGACT TTGAATGGAG AAAAAATTGA TGTATCTCAG CGATPCTTCT |
| 2351 | TTGCTTTAGG GGGCATGTTC TAA |

The PSORT algorithm predicts outer membrane (0.7658).

The protein was expressed in E. coli and purified as GST-fusion (FIG. 74A), his-tag and his-tag/GST-fusion products. The recombinant proteins were used to immunise mice, whose sera were used in a Western blot (FIG. 74B) and for FACS analysis (FIG. 74C).

The cp6576 protein was also identified in the 2D-PAGE experiment (Cpn0300).

These experiments show that cp6576 is a surface-exposed and immunoaccessible protein, and that it is a useful immunogen. These properties are not evident from the sequence alone.

### Example 75

The following C. pneumoniae protein (PID 4376607) was expressed <SEQ ID 149; cp6607>:

| | |
|---|---|
| 1 | MNKRQKDLK ICVIISTLIL VGIFARAPRG DTFKTFLKSE |
| | EAIIYSNQCN |
| 51 | EDMRKILCDA IEHADEEIFL RIYNLSEPKI QQSLTRQAQA |
| | KNKVTIYYQK |
| 101 | FKIPQILKQA SNVTLVEQPP AGRKLMHQKA LSIDKKDAWL |
| | GSANYTNLSL |
| 151 | RLDNNLILGM HSSELCDLII TNTSGDFSIK DQTGKYFVLP |
| | QDRKIAIQAV |
| 201 | LEKIQTAQKT IQVAMFALTH SEIIQALHQA KQRGIHVDII |
| | IDRSHSKLTF |
| 251 | KQLRQLNINK DFVSINTAPC TLHHKFAVID NKTLLAGSIN |
| | WSKGRFSLND |
| 301 | ESLIILENLT KQQNQKLRNI WRDLAXHSEH PTVDDEEKEI |
| | IEKSLPVEEQ |
| 351 | EAA* |

A predicted signal peptide is highlighted.

The cp6607 nucleotide sequence <SEQ ID 150> is:

| | |
|---|---|
| 1 | ATGAATAAAA GACAAAAAGA TAAATTAAAA ATCTGTGTTA |
| | TTATTACCAC |
| 51 | GTTGATTTTA GTAGGAATTT TTGCAAGAGC TCCTCGTGGT |
| | GACACTTTTA |
| 101 | AGACTTTTTT AAAGTCTGAA GAAGCTATCA TCTACTCAAA |
| | TCAATGCAAT |
| 151 | GAGCACATGC GTAAAATTCT ATGCCATGCA ATAGAACACC |
| | CTGATCAAGA |
| 201 | GATCTTCCTA CGTATTTATA ACCTCTCAGA ACCCAAGATC |
| | CAACAGAGTT |
| 251 | TAACTCGACA AGCTCAAGCA AAAAACAAAG TTACGATCTA |
| | CTATCAAAAA |
| 301 | TTTAAAATTC CCCAAATCTT AAAGCAAGCC AGCAATGTAA |
| | CTTTAGTCGA |
| 351 | GCAACCTCCA GCAGGGCGTA AACTGATGCA TCAAAAAGCT |
| | CTTTCCATAG |
| 401 | ATAAGAAAGA TGCTTGGCTA GGATCTGCGA ACTACACCAA |
| | TCTTTTTCTA |
| 451 | CGTTTAGATA ATAATCTCAT TCTAGGAATG CATAGCTCGG |
| | AGCTCTGTGA |
| 501 | TCTCATTATC ACAAATACCT CTGGAGACTT TTCTATAAAG |
| | GATCAAACAG |
| 551 | GAAAGTATTT TGTTCTTCCT CAAGATCGTA AAATTGCAAT |
| | ACAAGCTGTA |
| 601 | CTCGAAAAAA TCCAGACAGC TCAGAAAACC ATCCAAGTTG |
| | CTATGTTTGC |
| 651 | TCTGACCCAC TCGGAGATTA TTCAAGCCTT ACATCAAGCA |
| | AAACAACGAG |
| 701 | GAATCCATGT AGATATTATC ATTGATAGAA GTCATAGCAA |
| | ACTTACTTTT |
| 751 | AAGCAATTAC GACAATTAAA TATCAATAAA GACTTTGTTT |
| | CTATAAATAC |
| 801 | CGCACCCTGT ACTCTTCACC ATAAGTTTGC AGTTATAGAT |
| | AATAAAACTC |
| 851 | TACTTGCAGG ATCTATAAAT TGGTCTAAAG GAAGATTCTC |
| | CTTAAATGAT |
| 901 | GAAAGCTTGA TCATACTGGA AAACCTGACC AAACAACAAA |
| | ATCAGAAACT |
| 951 | TCGAATGATT TGGAAAGATC TAGCTAAGCA TTCAGAACAT |
| | CCTACAGTAG |
| 1001 | ACGATGAAGA AAAAGAAATT ATAGAAAAAA GTCTTCCAGT |
| | AGAAGAGCAA |
| 1051 | GAAGCAGCGT GA |

The PSORT algorithm predicts periplasmic (0.934).

The protein was expressed in E. coli and purified as a his-tagged product (FIG. 75A) and also as a GST-fusion. The GST-fusion protein was used to immunise mice, whose sera were used in a Western blot (FIG. 75B) and for FACS analysis.

These experiments show that cp6607 is a surface-exposed and immunoaccessible protein, and that it is a useful immunogen. These properties are not evident from the sequence alone.

### Example 76

The following C. pneumoniae protein (PID 4376624) was expressed <SEQ ID 151; cp6624>:

| | |
|---|---|
| 1 | MDAKMGYIFK VMRWIFCFVA CGITFGCTNS GFQNANSRPC |
| | ILSMNRMIHD |
| 51 | CVBRVVGNRL ATAVLIKGSL DPHAYEMVKG DKDRIAGSAV |
| | IFCNGLGLEH |
| 101 | TLSLREHLEN NPNSVKLGER LIARGAFVPL EEDGICDPUI |
| | WMDLSIWKEA |
| 151 | VIEITEVLIE KFPEWSAEFK ANSEELVCEM SILDSWAIQC |
| | LSTIPBNLRY |
| 201 | LVSGHNAFSY FTRRYLATPE EVASGAWRSR CISPEGLSPE |
| | AQISVRDIMA |
| 251 | VVDYINEHDV SVVFPEDTLN QDALKKIVSS LKKSHLVRLA |
| | QKPLYSDNVD |
| 301 | DNYFSTFKHN VCLITEELGG VALECQR* |

The cp6624 nucleotide sequence <SEQ ID 152> is:

| | |
|---|---|
| 1 | ATGGATGCGA AAATGGGATA TATATTTAAA GTGATGCGTT |
| | GGATTTTCTG |
| 51 | TTTCGTGGCA TGTGGTATAA CTTTTGGATG TACCAATTCT |
| | GGGTTTCAGA |
| 101 | ATGCAAATTC ACGTCCTTGT ATACTATCCA TGAATCGCAT |
| | GATTCATGAT |
| 151 | TGTGTTGAAA GAGTCGTGGG GAATAGGCTT GCTACCGCTG |
| | TTTTGATCAA |
| 201 | AGGATCCTTA GACCCTCATG CGTATGAGAT GGTTAAAGGG |
| | GATAAGGACA |
| 251 | AGATTGCTGG AAGTGCCGTA ATTTTTTGTA ACGGCCTGGG |
| | TCTTGAGCAT |
| 301 | ACATTAAGTT TGCGGAAGCA TTTAGAAAAT AATCCCAATA |
| | GTGTCAAGTT |
| 351 | AGGGGAGCGG TTCATAGCGC GTGGGGCCTT TGTTCCTCTA |
| | GAAGAAGACG |
| 401 | GTATTTGCGA TCCTCATATC TGGATGGATC TTTCTATTTG |
| | GAAGGAAGCT |
| 451 | GTCATAGAAA TTACAGAAGT TCTCATTGAA AAGTTCCCTG |
| | AATGGTCTGC |
| 501 | TGAATTTAAA GCAAATAGTG AGGAACTTGT TTGTGAAATG |
| | TCTATTTTAG |
| 551 | ATTCTTGGGC GAAACAATGC TTGAGCACAA TTCCTGAAAA |
| | TTTACGGTAT |
| 601 | CTTGTCTCAG GTCATAATGC GTTCAGTTAC TTTACACGTC |
| | GCTATTTAGC |
| 651 | TACTCCTGAA GAAGTGGCTT CCGGAGCATG GAGGTCTCGT |
| | TATATGGCTC |
| 701 | CTGAGGGTCT ATCTCCAGAA GCTCAAATCA GTGTTCGTGA |
| | TATTATGGCG |
| 751 | GTTGTAGATT ATATTAATGA GCATGATGTC AGTGTGGTTT |
| | TCCCTGAGGA |
| 801 | TACTCTGAAC CAAGATGCGT TGAAAAAAAT TGTTTCTTCT |
| | CTGAAGAAAA |
| 851 | GTCATTTAGT TCGTCTAGCT CAAAAACCAT TGTATAGTGA |
| | TAATGTGGAC |
| 901 | GACAATTATT TTAGCACCTT TAAACATAAT GTCTGCCTTA |
| | TCACAGAAGA |
| 951 | ATTAGGAGGG GTGGCTCTTG AATGTCAAAG ATGA |

The PSORT algorithm predicts inner membrane (0.168).

The protein was expressed in E. coli and purified as a his-tag product (FIG. 76A). The recombinant protein was used to immunise mice, whose sera were used in a Western blot (FIG. 76B) and for FACS analysis.

The cp6624 protein was also identified in the 2D-PAGE experiment.

These experiments show that cp6624 is a surface-exposed and immunoaccessible protein, and that it is a useful immunogen. These properties are not evident from the sequence alone.

### Example 77

The following C. pneumoniae protein (PID 4376728) was expressed <SEQ ID 153; cp6728>:

| | |
|---|---|
| 1 | MKSSVSWLFF SSIPLFSSLS IVAAEVTLDS SNNSYDGSNG |
| | TTFTVFSTTD |
| 51 | AAAGTTYSLL SDVSFQNAGA LGIPLASGCF LEAGGDLTFQ |
| | GNQHALKFAF |
| 101 | INAGSSAGTV ASTSAADKNL LFNDFSRLSI ISCPSLLLSP |
| | TGQCALRSVG |
| 151 | NLSLTGNSQI IFTQNFSSDN GGVINTKNFL LSGTSQFASF |
| | SRNQAFTGKQ |
| 201 | GGVVYATGTI TIENSPGIVS FSQNLAKGSG GATYSTDNCS |
| | ITDNFQVIFD |
| 251 | GNSAWEAAQA QGGAICCTTT DKTVTLTGNK NLSPTNNTAL |
| | TYGGAISGLK |
| 301 | VSISAGGPTL FQSNISGSSA GQGGGGAINI ASAGELALSA |
| | TSGDITFNNN |
| 351 | QVTNGSTSTR NAINIIDTAK VTSIRAATGQ SIYFYDPITN |
| | PGTAASTDTL |
| 401 | NLNLADANSE IEYGGAIVFS GEKLSPTEKA IAANVTSTIR |
| | QPAVLARGDL |
| 451 | VLRDGVTVTF KDLTQSPGSR ILMDGGTTLS AKEANLSLNG |
| | LAVNLSSLDG |
| 501 | TNKAALKTEA ADKNISLSGT IALIDTEGSF YENHNLKSAS |
| | TYPLLELTTA |
| 551 | GANGTITLGA LSTLTLQEPE THYGYQGNWQ LSWANATSSK |
| | IGSINWTRTG |
| 601 | YIPSPERKSN LPLNSLWGNF IDIRSINQLI ETKSSGEPFE |
| | RELWLSGIAN |
| 651 | FFYRDSMPTR HGFRHISGGY ALGITATTPA EDQLTFAFCQ |
| | LEARDRNHIT |
| 701 | GKNHGDTYGA SLYFHHTEGL FDIANFLWGK ATRAPWVLSE |
| | ISQIIPLSPD |
| 751 | AKFSYLHTDN HMKTYYTDNS IIKGSWRNDA FCADLGASLP |
| | FVISVPYLLK |
| 801 | EVEPFVKVQY IYAHQQDFYE RHAEGRAPNK SELINVEIPI |
| | GVTFERDSKS |
| 851 | EKGTYDLTIM YILDAYRRNP KCQTSLIASD ANWMAYGTNL |
| | ARQGFSVRAA |
| 901 | NHFQVNPHME IFGQFAFEVR SSSRNYNTNL GSKPCF* |

The cp6728 nucleotide sequence <SEQ ID 154> is:

| | |
|---|---|
| 1 | ATGAAGTCCT CTGTCTCTTG GTTGTTCTTT TCTTCAATCC |
| | CGCTCTTTTC |
| 51 | ATCGCTCTCT ATAGTCGCGG CAGAGGTGAC CTTAGATAGC |
| | AGCAATAATA |
| 101 | GCTATGATGG ATCTAACGGA ACTACCTTCA CGGTCTTTTC |
| | CACTACGGAC |
| 151 | GCTGCTGCAG GAACTACCTA TTCCTTACTT TCCGACGTAT |
| | CCTTTCAAAA |
| 201 | TGCAGGGGCT TTAGGAATTC CCTTAGCCTC AGGATGCTTC |
| | CTAGAAGCGG |
| 251 | GCGGCGATCT TACTTTCCAA GGAAATCAAC ATGCACTGAA |
| | GTTTGCATTT |
| 301 | ATCAATGCGG GCTCTAGCGC TGGAACTGTA GCCAGTACCT |
| | CAGCAGCAGA |
| 351 | TAAGAATCTT CTCTTTAATG ATTTTTCTAG ACTCTCTATT |
| | ATCTCTTGTC |
| 401 | CCTCTCTTCT TCTCTCTCCT ACTGGACAAT GTGCTTTAAA |
| | ATCTGTGGGG |
| 451 | AATCTATCTC TAACTGGCAA TTCCCAAATT ATATTTACTC |
| | AGAACTTCTC |
| 501 | GTCAGATAAC GGCGGTGTTA TCAATACGAA AAACTTCTTA |
| | TTATCAGGGA |
| 551 | CATCTCAGTT TGCGAGCTTT TCGAGAAACC AAGCCTTCAC |
| | AGGGAAGCAA |
| 601 | GGCGGTGTAG TTTACGCTAC AGGAACTATA ACTATCGAGA |
| | ACAGCCCTGG |
| 651 | GATAGTTTCC TTCTCTCAAA ACCTAGCGAA AGGATCTGGC |
| | GGTGCTCTCT |
| 701 | ACAGCACTGA CAACTGTTCG ATTACAGATA ACTTTCAAGT |
| | GATCTTTGAC |
| 751 | GGCAATAGTG CTTGGGAAGC CGCTCAAGCT CAGGGCGGGG |
| | CTATTTGTTG |
| 801 | CACTACGACA GATAAAAGAG TGACTCTTAC TGGGAACAAA |
| | AACCTCTCTT |
| 851 | TCACAAATAA TACAGCATTG ACATATGGCG GAGCCATCTC |
| | TGGACTCAAG |
| 901 | GTCAGTATTT CCGCTGGAGG TCCTACTCTA TTTCAAAGTA |
| | ATATCTCAGG |
| 951 | AAGTAGCGCC GGTCAGGGAG GAGGAGGAGC GATCAATATA |
| | GCATCTGCTG |
| 1001 | GGGAACTCGC TCTCTCTGCT ACTTCTGGAG ATATTACCTT |
| | CAATAACAAC |
| 1051 | CAAGTCACCA ACGGAAGCAC AAGTACAAGA AACGCAATAA |
| | ATATCATTGA |
| 1101 | TACCGCTAAA GTCACATCGA TACGAGCTGC TACGGGGCAA |
| | TCTATCTATT |
| 1151 | TCTATGATCC CATCACAAAT CCAGGAACCG CAGCTTCTAC |
| | CGACACATTG |
| 1201 | AACTTAAACT TAGOAGATCC GAACAGTGAG ATCGAGTATG |
| | GGGGTGCGAT |
| 1251 | TGTCTTTTCT GGAGAAAAGC TTTCCCCTAC AGAAAAAGCA |
| | ATCGCTGCAA |
| 1301 | ACGTCACCTC TACTATCCGA CAACCTGCAG TATTAGCGCG |
| | GGGAGATCTT |
| 1351 | GTACTTCGTG ATGGAGTCAC CGTAACTTTC AAGGATCTGA |
| | CTCAAAGTCC |
| 1401 | AGGATCCCGC ATCTTAATGG ATGGGGGGAC TACACTTAGT |
| | GCTAAAGAGG |
| 1451 | CAAATCTTTC GCTTAATCGC TTAGCAGTAA ATCTCTCCTC |
| | TTTAGATGGA |
| 1501 | ACCAACAAGG CAGCTTTAAA AACAGAAGCT GCAGATAAAA |
| | ATATCAGCCT |
| 1551 | ATCGGGAACG ATTGCGCTTA TTGACACGGA AGGGTCATTC |
| | TATGAGAATC |
| 1601 | ATAACTTAAA AAGTGCTAGT ACCTATCCTC TTCTTGAACT |
| | TACCACCGCA |
| 1651 | GGAGCCAACG GAACGATTAC TCTGGGAGCT CTTTCTACCC |
| | TGACTCTTCA |
| 1701 | AGAACCTGAA ACCCACTACG GGTATCAAGG AAACTGGCGG |
| | TTGTCTTGGG |
| 1751 | CAAATGCAAC ATCCTCAAAA ATAGGAAGCA TCAACTGGAC |
| | CCGTACAGGA |
| 1801 | TACATTCCTA GTCCTGAGAG AAAAAGTAAT CTCCCTCTAA |
| | ATAGCTTATG |
| 1851 | GGGAAACTTT ATAGATATCG GCTCGATCAA TCAGCTTATA |
| | GAAACCAAGT |
| 1901 | CCAGTGGGGA GCCTTTTGAG CGTGAGCTAT GGCTTTCAGG |
| | AATTGCGAAT |
| 1951 | TTCTTCTATA GAGATTCTAT GCCCACCCGC CATGGTTTCC |
| | GCCATATCAG |
| 2001 | CGGGGGTTAT GCACTAGGGA TCACAGCAAC AACTCCTGCC |
| | GAGGATCAGC |
| 2051 | TTACTTTTGC CTTCTGCCAG CTCTTTGOTA GAGATCGCAA |
| | TCATATTACA |
| 2101 | GGTAAGAACC ACGGAGATAC TTACGGTGCC TCTTTGTATT |
| | TCCACCATAC |
| 2151 | AGAAGGGCTC TTCGACATCG CCAATTTCCT CTGGGGAAAA |
| | GCAACCCGAG |
| 2201 | CTCCCTGGGT GCTCTCTGAG ATCTCCCAGA TCATTCCTTT |
| | ATCGTTCGAT |
| 2251 | GCTAAATTCA GTTATCTCCA TACAGACAAC CACATGAAGA |
| | CATATTATAC |
| 2301 | CGATAACTCT ATCATCAAGG GTTCTTGGAG AAACGATGCC |
| | TTCTGTGCAG |
| 2351 | ATCTTGGAGC TAGCCTGCCT TTTGTTATTT CCGTTCCGTA |
| | TCTTCTGAAA |
| 2401 | GAAGTCGAAC CTTTTGTCAA AGTACAGTAT ATCTATGCGC |
| | ATCAGCAAGA |
| 2451 | CTTCTACGAG CGTCATGCTG AAGGACGCGC TTTCAATAAA |
| | AGCGAGCTTA |
| 2501 | TCAACGTAGA GATTCCThTA GGCGTCACCT TCGAAAGAGA |
| | CTCAAAATCA |
| 2551 | GAAAAGGGAA CTTACGATCT ThCTCTTATG TATATACTCG |
| | ATGCTTACCG |
| 2601 | ACGCAATCCT AAATGTCAAA CTTCCCTAAT AGCTAGCGAT |
| | GCTAACTGGA |
| 2651 | TGGCCTATGG TACCAACCTC GCACGACAAG GTTTTTCTGT |
| | TCGTGCTGCG |
| 2701 | AACCATTTCC AAGTGAACCC CCACATGGAA ATCTTCGGTC |
| | AATTCGCTTT |
| 2751 | TGAAGTACGA AGTTCTTCAC GAAATTATAA TACAAACCTA |
| | GGCTCTAAGT |
| 2801 | TTTGTTTCTA G |

The PSORT algorithm predicts inner membrane (0.187).

The protein was expressed in E. coli and purified as a GST-fusion product (FIG. 77A). The recombinant protein was used to immunise mice, whose sera were used in a Western blot (FIG. 77B) and for FACS analysis.

The cp6728 protein was also identified in the 2D-PAGE experiment.

These experiments show that cp6728 is a surface-exposed and immunoaccessible protein, and that it is a useful immunogen. These properties are not evident from the sequence alone.

### Example 78

The following C. pneumoniae protein (PID 4376847) was expressed <SEQ ID 155; cp6847>:

| | |
|---|---|
| 1 | MFVMKKLVRL CVVLLSLLPN VLFSSDLLRE EGIKKMMDKL |
| | IEYHVDAQEV |
| 51 | STKILSRSLS SYIQSFDPHK SYLSNQEVAV FLQSPETKKR |
| | LLKNYKAGNF |
| 101 | AIYRNINQLI HESILRARQW RNEWVKNPKE LVLEASSYQI |
| | SKQPMQWSKS |
| 151 | LDEVKQRQEA LLLSYLSLHL AGASSSRYEG KEEQLAALCL |
| | RQIENHENVY |
| 201 | LGINDHGVAM DRDEEAYQFH IRVVKALAHS LDAHTAYFSK |
| | DEALAMRIQL |
| 251 | EKGMCGIGVV LKEDXDGVVV REIIPGGPAA KSGDLQLGDI |
| | IYRVDGKDIE |
| 301 | HLSFRGVLDC LRGGHGSTVV LDIHRGESDH TIALRREKIL |
| | LEDRRVDVSY |
| 351 | EPYGDGVXGK VTLHSFYEGE NQVSSEQDLR RAIQGLKEKN |
| | LLGLVLDIRE |
| 401 | NTGGPLSQAI KVSGLFETNQ VVVVSRYADG TMRCYRTVSP |
| | KKFYDGPLAI |
| 451 | LVSKSSASAA EIVAQTLQDY GVALVVGDEQ TYGKGTIQHQ |
| | TITGDASQDD |
| 501 | CFKVTVGKYY SPSGKSTQLQ GVKSDILIPS LYAEDRLGER |
| | FLEHPLPADC |
| 551 | CDNVLRDPLT DLDTQTRPWF QXYYLPNLQK QETLWREMLP |
| | QLTKNSEQRL |
| 601 | SENSNFQAFL SQIKSSEKTD LSYGSNDLQL EESINILKDM |
| | ILLQQCRK* |

A predicted signal peptide is highlighted.

The cp6847 nucleotide sequence <SEQ ID 156> is:

| | |
|---|---|
| 1 | ATGTTCGTAA TGAAAAAACT TGTCCGTCTA TGCGTAGTTC |
| | TTCTTTCTTT |
| 51 | ACTTCCGAAT GTATTATTTT CTTCGGATCT TTTACGAGAA |
| | GAGGGCATCA |
| 101 | AAAAGATGAT GGACAAGCTG ATCGAGTATC ATGTCGAGGC |
| | TCAAGAGGTT |
| 151 | TCTACGGATA TACTCTCGCG TTCTTTATCT AGTTACATTC |
| | AATCTTTTGA |
| 201 | TCCTCATAAA TCTTATCTTT CAAACCAAGA GGTTGCAGTT |
| | TTTCTACAGT |
| 251 | CTCCGGAAAC AAAGAAACGT CTCTTAAAGA ATTATAAGGC |
| | AGGCAACTTT |
| 301 | GCTATTTATC GCAACATCAA TCAATTAATT CATGAGAGTA |
| | TTCTTCGTGC |
| 351 | CAGGCAGTGG AGAAACGAAT GGGTTAAGAA TCCAAAAGAG |
| | CTTGTATTGG |
| 401 | AGGCATCCTC ATATCAGATA TCGAAGCAAC CTATGCAATG |
| | GAGCAAATCT |
| 451 | TTAGACGAAG TGAAGCAGAG ACAACGCGCT CTACTCCTTT |
| | CCTATCTTTC |
| 501 | TTTACATCTT GCTGGAGCTT CTTCCTCTCG TTATGAGGGT |
| | AAAGAAGAGC |
| 551 | AGCTTGCTGC TCTGTGTCTA CGTCAAATCG AGAACCATGA |
| | GAATGTATAT |
| 601 | TTAGGTATCA ACGATCATGG TGTTGCTATG GATCGGGATG |
| | AAGAAGCCTA |
| 651 | CCAATTCCAT ATCCGTGTTG TTAAAGCTTT AGCTCATAGC |
| | TTAGATGCAC |
| 701 | ATACGGCGTA TTTCAGTAAG GACGAAGCGT TGGCGATGCG |
| | AATCCAACTA |
| 751 | GAAAAAGGCA TGTGTGGAAT TGGTGTTGTT CTGAAGGAAG |
| | ATATTGATGG |
| 801 | AGTTGTTGTT AGAGAAATCA TTCCTGGGGG ACCTGCGGCT |
| | AAATCTGGGG |
| 851 | ATCTTCAGCT TGGAGATATC ATCTATCGGG TGGATGGCAA |
| | GGATATCGAG |
| 901 | CATCTTTCTT TCCGCGGTGT TTTAGATTGT TTACGTGGAG |
| | GTCATGGCTC |
| 951 | TACTGTAGTC TTAGATATCC ATCGTGGGGA GAGCGATCAT |
| | ACGATCGCCT |
| 1001 | TGAGAAGGGA GAAAATCCTT TTAGAAGACC GTCGTGTGGA |
| | TGTTTCCTAT |
| 1051 | GAGCCTTATG GAGATGGTGT GATTGGGAAA GTTACGTTAC |
| | ATTCTTTTTA |
| 1101 | TGAAGGAGAA AATCAGGTTT CTAGTGAACA AGATCTACGT |
| | CGAGCGATTC |
| 1151 | AGGGATTAAA GGAGAAGAAC CTTCTTGGAT TAGTTTTAGA |
| | TATCCGAGAA |
| 1201 | AATACGGGTG GATTTTTATC TCAAGCGATC AAAGTTTCTG |
| | GTTTATTTAT |
| 1251 | GACCAATGGC GTTGTGGTTG TATCTCGCTA TGCTGATGGT |
| | ACCATGAAGT |
| 1301 | GCTACCGCAC AGTATCTCCT AAAAAATTCT ATGATGGTCC |
| | TTTGGCTATT |
| 1351 | TTAGTATCTA AAAGTTCCGC ATCAGCAGCG GAGATTGTAG |
| | CACAAACTCT |
| 1401 | CCAAGATTAT GGAGTTGCTT TAGTTGTTGG AGATGAGCAG |
| | ACCTATGGGA |
| 1451 | AGGGAACGAT TCAGCATCAA ACAATTACTG GAGATGCCTC |
| | TCAGGACGAT |
| 1501 | TGTTTTAAGG TTACTGTAGG GAAATATTAT TCCCCTTCTG |
| | GGAAATCGAC |
| 1551 | TCAACTTCAG GGAGTAAAAT CCGATATTTT AATTCCTTCT |
| | CTCTATGCTG |
| 1601 | AAGATCGTCT AGGAGAGCGT TTTCTAGAGC ATCCCTTACC |
| | TGCAGATTGC |
| 1651 | TGTGATAATG TACTTCACGA TCCTCTCACG GACTTGGATA |
| | CTCAAACACG |
| 1701 | TCCTTGGTTT CAAAAATACT ATCTTCCTAA TCTACAAAAG |
| | CAAGAGACTC |
| 1751 | TTTGGAGAGA GATGCTACCT CAGCTTAGGA AAAACAGTGA |
| | GCAAAGGCTT |
| 1801 | TCTGAGAATT CGAATTTTCA GGCATTTTTG TCGCAGATAA |
| | AATCATCTGA |
| 1851 | AAAAACGGAC CTATCCTATG GTTCCAATGA TTTACAATTG |
| | GAAGAGTCGA |
| 1901 | TAAACATTTT GAAGGACATG ATTTTATTAC AACAGTGTAG |
| | AAAATAA |

The PSORT algorithm predicts periplasmic (0.932).

The protein was expressed in E. coli and purified as a GST-fusion product (FIG. 78A). The recombinant protein was used to immunise mice, whose sera were used in a Western blot (FIG. 78B) and for FACS analysis.

These experiments show that cp6847 is a surface-exposed and immunoaccessible protein, and that it is a useful immunogen. These properties are not evident from the sequence alone.

### Example 79

The following C. pneumoniae protein (PID 4376969) was expressed <SEQ ID 157; cp6969>:

| | |
|---|---|
| 1 | MRLFSLGTIY LFFSLALSSC CGYSILNSPY HLSSLGKSLL |
| | QERIFIAPIK |
| 51 | EDPHGQLCSA LTYELSKRSF AISGRSSCAG YTLKVELLNG |
| | IDKNIGFTYA |
| 101 | PNKLGDKTHR HFIVSNBGRL SLSAKVQLIN NDTQEVLIDQ |
| | CVARESVDFD |
| 151 | FEPDLGTANA HEFALGQFEM HSEAIKSARR ILSIRLAETI |
| | AQQVYYDLF* |

A predicted signal peptide is highlighted.

The cp6969 nucleotide sequence <SEQ ID 158> is:

| | |
|---|---|
| 1 | ATGAGATTGT TTTCTTTAGG CACGATTTAT CTTTTTTTTT |
| | CTCTAGCACT |
| 51 | TTCGTCATGC TGTGGTTACT CTATTTTAAA CAGCCCGTAT |
| | CACTTATCGT |
| 101 | CTTTAGGTAA GTCTTTATTA CAGGAAAGAA TTTTCATTGC |
| | TCCCATAAAA |
| 151 | GAAGATCCTC ATGGTCAGCT CTGCTCAGCT CTAACTTATG |
| | AGCTTAGTAA |
| 201 | GCGTTCTTTT GCTATCTCTG GAAGGAGTTC TTGCGCAGCC |
| | TATACTCTTA |
| 251 | AAGTAGAGCT TCTGAATGGT ATTGACAAGA ATATAGGTTT |
| | TACGTATGCC |
| 301 | CCAAATAAAC TCGGAGATAA GACTCACAGG CATTTTATAG |
| | TCTCTAATGA |
| 351 | AGGCAGACTA TCACTATCTG CAAAAGTACA GCTTATCAAT |
| | AATGACACTC |
| 401 | AAGAAGTCCT TATAGACCAA TGTGTTGCTC GAGAGTCTGT |
| | AGACTTTGAC |
| 451 | TTTGAGCCTG ACTTAGGAAC AGCAAACGCT CATGAATTTC |
| | CTTTAGGCCA |
| 501 | ATTTGAAATG CATAGTGAAG CCATAAAAAG TGCTCGCCGT |
| | ATACTATCTA |
| 551 | TACGCCTAGC CGAGACGATT GCTCAACAGG TACACTATGA |
| | CCTTTTTTGA |

The PSORT algorithm predicts inner membrane (0.126).

The protein was expressed in E. coli and purified as a GST-fusion product (FIG. 79A). The recombinant protein was used to immunise mice, whose sera were used in a Western blot (FIG. 79B) and for FACS analysis.

These experiments show that cp6969 is a surface-exposed and immunoaccessible protein, and that it is a useful immunogen. These properties are not evident from the sequence alone.

### Example 80

The following C. pneumoniae protein (PID 4377109) was expressed <SEQ ID 159; cp7109>:

| | |
|---|---|
| 1 | MKKTCCQNYR SIGVVFSVVL FVLTTQTLFAGHFIDIGTSG |
| | LYSWARGVSG |
| 51 | DGRVVVGYEG GNAFKYVDGE KFLLEGLVPR SEALVFKASY |
| | DGSVIIGISD |
| 101 | QDPSCRAVKW VNGALVDLGI FSEGMQSFAE GVSSDGWPIV |
| | GCLYSDDTET |
| 151 | NFAVKWDETG MVVLPNLPED RHSCAWDASE DGSVIVGDAM |
| | GSEEIAKAVY |
| 201 | WKDGEQHLLS NIPGAKRSSA HAVSKDGSFI VGEFISEENE |
| | VHAFVYHNGV |
| 251 | IKDIGTLGGD YSVATGVSRD GKVIVGHSTR TDGEYRAFKY |
| | VDGRMIDLGT |
| 301 | LGGSASFAFG VSDDGKTIVG KFETELGECH AFIYLDD* |

A predicted signal peptide is highlighted.

The cp7109 nucleotide sequence <SEQ ID 160> is:

| | |
|---|---|
| 1 | ATGAAAAAGA CATGTTGCCA AAATTACAGA TCGATAGGCG |
| | TTGTGTTCTC |
| 51 | TGTGGTACTT TTCGTTCTTA CAACACAGAC GCTGTTTGCA |
| | GGACATTTTA |
| 101 | TTGATATTGG AACTTCTGGA TTATATTCTT GGGCTCGAGG |
| | TGTATCTGGA |
| 151 | GATGGCCGCG TTGTCGTAGG TTATGAAGGT GGCAATGCAT |
| | TTAAATATGT |
| 201 | TGATGGTGAG AAATTTCTGT TAGAAGGTTT GGTCCCGAGA |
| | TCCGAGGCCT |
| 251 | TGGTATTTAA AGCTTCTTAT GATGGCTCTG TAATTATAGG |
| | AATCTCGGAT |
| 301 | CAAGATCCGT CTTGCCGCGC TGTGAAGTGG GTAAACGGTG |
| | CACTTGTTGA |
| 351 | TCTTGGAATA TTTTCTGAGG GAATGCAATC TTTTGCAGAG |
| | GGTGTTTCCA |
| 401 | GTGATGGAAA GACGATTGTA GGGTGCCTAT ATAGTGATGA |
| | TACAGAGACA |
| 451 | AACTTTGCTG TGAAGTGGGA TGAAACAGGA ATGGTTGTTC |
| | TCCCTAACTT |
| 501 | ACCAGAAGAT CGACATTCTT GCGCTTGGGA TGCCTCTGAA |
| | GATGGCTCTG |
| 551 | TGATTGTAGG GGACGCCATG GGTAGCGAGG AAATTGCCAA |
| | GGCAGTGTAC |
| 601 | TGGAAGGACG GTGAACAACA TCTGCTTTCT AATATCCCAG |
| | GAGCTAAAAG |
| 651 | ATCGTCAGCA CATGCAGTTT CTAAAGATGG ATCTTTTATC |
| | GTAGGCGAGT |
| 701 | TCATCAGTGA AGAAAATGAA GTTCATGCCT TTGTTTATCA |
| | CAACGGTGTT |
| 751 | ATCAAAGATA TCGGGACTTT AGGAGGAGAT TACTCTGTAG |
| | CAACTGGAGT |
| 801 | TTCTAGGGAT GGTAAGGTCA TCGTGGGTCA TTCTACAAGA |
| | ACAGATGGTG |
| 851 | AATACCGTGC ATTTAAATAT GTGGATGGAA GAATGATAGA |
| | TTTGGGGACT |
| 901 | TTAGGAGGTT CAGCATCTTT TGCTTTTGGT GTTTCTGACG |
| | ATGGCAAAAC |
| 951 | AATCGTAGGA AAATTTGAAA CAGAGCTAGG AGAATGTCAT |
| | GCCTTTATCT |
| 1001 | ACCTTGATGA TTAG |

The PSORT algorithm predicts outer membrane (0.887).

The protein was expressed in E. coli and purified as a GST-fusion product (FIG. 80A). The recombinant protein was used to immunise mice, whose sera were used in a Western blot (FIG. 80B) and for FACS analysis.

These experiments show that cp7109 is a surface-exposed and immunoaccessible protein, and that it is a useful immunogen. These properties are not evident from the sequence alone.

### Example 81

The following C. pneumoniae protein (PID 4377110) was expressed <SEQ ID 161; cp7110>:

| | |
|---|---|
| 1 | MAAIKQILRS MLSQSSLWMV LFSLYSLSGY CYVITDKPED |
| | DFHSSSAVKW |
| 51 | DHWGKTTLSR LSNXKASAKA VSGTGATTVG FIKDTWSRTY |
| | AVRWNYWGPK |
| 101 | ELPTSSWVKK SKATGISSDG SIIAGIVENE LSQSFAVTWK |
| | NNEMYLLPST |
| 151 | WAVQSKAYGI SSDGSVIVGS AKDAWSRTFA VKWTGHEAQV |
| | LPVGWAVKSV |
| 201 | ANSVSANGSI IVGSVQDASG ILYAVKWEGN TITHLGTLGG |
| | YSAIAKAVSN |
| 251 | NGKVIVGRSE TYYGEVHAPC HKNGVMSDLG TLGGSYSAAK |
| | GVSATGKVIV |
| 301 | GMSTTANGKL HAFKYVGGRM IDLGEYSWKE ACENAVSIDG |
| | EIIVGVQSE* |

A predicted signal peptide is highlighted.

The cp7110 nucleotide sequence <SEQ ID 162> is:

| | |
|---|---|
| 1 | ATGGCAGCTA TAAAACAAAT TTTACGTTCT ATGCTATCTC |
| | AGAGTAGCTT |
| 51 | ATGGATGGTC CTATTTTCAT TATATTCTCT ATCTGGTTAT |
| | TGCTATGTAA |
| 101 | TTACAGACAA ACCAGAAGAT GACTTCCATT CTTCATCCGC |
| | AGTAAAATGG |
| 151 | GATCATTGGG GAAAGACAAC TCTCTCAAGA TTATCAAATA |
| | AAAAAGCCTC |
| 201 | TGCAAAAGCT GTTTCAGGAA CTGGTGCTAC AACTGTCGGC |
| | TTTATAAAAG |
| 251 | ACACTTGGTC TCGAACATAC GCAGTAAGAT GGAATWATTG |
| | GGGGACCAAA |
| 301 | GAACTCCCTA CCAGCTCATG GGTAAAAAAA TCAAAAGCAA |
| | CAGGAATCTC |
| 351 | CTCTGATGGG TCTATAATCG CGGGGATTGT CGAGAATGAG |
| | CTTTCTCAAA |
| 401 | GTTTCGCAGT CACATGGAAA AACAATGAAA TGTATTTGCT |
| | CCCTTCCACA |
| 451 | TGGGCAGTGC AATCTAAAGC GTATGGAATT TCTTCTGATG |
| | GCTCTGTTAT |
| 501 | TGTAGGGAGT GCTAAGGATG CTTGGTCGCG AACTTTCGCT |
| | GTGAAGTGGA |
| 551 | CGGGACACGA GGCTCAGGTG TTACCAGTAG GCTGGGCTGT |
| | CAAATCTGTA |
| 601 | GCGAATTCTG TATCTGCCAA TGGATCTATA ATTGTAGGGT |
| | CTGTACAAGA |
| 651 | CGCCTCTGGA ATTCTTTATG CTGTAAAGTG GGAAGGGAAC |
| | ACTATTACAC |
| 701 | ATCTAGGAAC TTTAGGAGGC TATTCTGCCA TTGCAAAAGC |
| | TGTATCCAAT |
| 751 | AATGGCAACG TCATTGTAGG GAGATCCGAA ACATATTATG |
| | GAGAGGTCCA |
| 801 | TGCTTTCTGT CATAAGAATG GCGTCATGTC AGACCTCGGC |
| | ACCCTCGGAG |
| 851 | GATCTTATTC TGCAGCTAAG GGAGTCTCTG CAACTGGAAA |
| | AGTTATTGTC |
| 901 | GGTATGTCCA CAACAGCAAA TGGGAAATTG CATGCCTTTA |
| | AATATGTCGG |
| 951 | TGGAAGAATG ATCGACTTAG GAGAGTATAG CTGGAAAGAA |
| | GCGTGTGCAA |
| 1001 | ACGCTGTTTC TATTGATGGA GAAATTATTG TTGGAGTCCA |
| | ATCAGAATAA |

The PSORT algorithm predicts outer membrane (0.827).

The protein was expressed in E. coli and purified as a GST-fusion product (FIG. 81A). The recombinant protein was used to immunise mice, whose sera were used in a Western blot (FIG. 81B) and for FACS analysis.

These experiments show that cp7110 is a surface exposed and immunoaccessible protein, and that it is a useful immunogen. These properties are not evident from the sequence alone.

FIG. 191 shows a schematic representation of the structural relationships between of cp7105, cp7106, cp7107, cp7108, cp7109 and cp7110, each of which is identified herein. These six proteins may be grouped in a new family of related outer membrane-associated proteins. These proteins have a repeat structure in common (cf. the pmp family).

### Example 82

The following C. pneumoniae protein (PID 4377127) was expressed <SEQ ID 163; cp7127>:

| | |
|---|---|
| 1 | MVFFRNSLLH LVALSGMLCC SSGVALTIAE KMASLEHSGR |
| | GADDYEGMAS |
| 51 | FNANMREYSL QLSKLYEEAR KLRASGTEDE ALWKDLIERI |
| | GEVRGYLREI |
| 101 | EELWAAEIRE KGGNLEDYAL WNHPETTIYN LVTDYGTEDS |
| | IYLIPQEIGA |
| 151 | IKIATLSKFV VPKESFEDCL TQILSRLGIG VRQVNSWIKE |
| | LYMMRKEGCS |
| 201 | VAGVFSSRKD LEALPETAYI GFVLNSNVDA HTNQHVLKKF |
| | INPETTHVDV |
| 251 | IAGRVWIFGS AGEVGELLKI YNFVQSESIR QEYRVIPLTK |
| | IDPGEMISIL |
| 301 | NAAFREDLTK DVSEESLGLR VVPLQYQGRS LFLSGTAALV |
| | QQALTLIREL |
| 351 | EEGIENPTDK TVFWYNVKHS DPQELAALLS QVHDVFSGEN |
| | KASVGAADGC |
| 401 | GSQLNASIQI DTTVSSSAKD GSVKYGNFIA DSKTGTLIMV |
| | VEKEVLPRIQ |
| 451 | MLLKKLDVPK KMVRIEVLLF ERKLAHEQKS GLNLLRLGEE |
| | VCKKGCSPSV |
| 501 | SWAGGTGILE FLFKGSTGSS IVPGYDLAYQ FLMAQEDVRI |
| | NASPSVVTMN |
| 551 | QTPARIAVVD EMSIAVSSDK DKAQYNRAQY GIMIKMLPVI |
| | NVGEEDGKSY |
| 601 | ITLETDITFD TTGKNHDDRP DVTRRNITNK VRIADGETVI |
| | IGGLRCKQMS |
| 651 | DSHDGIPFLG DIPGIGKLFG MSSTSDSLTE MFVFITPKIL |
| | ENPVEQQERK |
| 701 | EEALLSSRPG EREEYYQALA ASEAAARAAH KKLENFPASG |
| | VSLSQVERQB |
| 751 | YDGC* |

A predicted signal peptide is highlighted.

The cp7127 nucleotide sequence <SEQ ID 164> is:

| | |
|---|---|
| 1 | ATGGTTTTTT TCCGTAATTC TTTACTGCAT TTAGTTGCCC |
| | TATCCGGAAT |
| 51 | GCTCTGTTGT TCTTCTGGAG TGGCTTTAAC GATAGCCGAG |
| | AAGATGGCTT |
| 101 | CTTTAGAGCA CTCGGGGAQA GGAGCAGACG ATTATGAGGG |
| | GATGGCTTCG |
| 151 | TTTAATGCCA ATATGAGGQA GTATAGCCTT CAGCTGAGCA |
| | AGTTGTATGA |
| 201 | GGAAGCACGA AAGCTACGCG CTTCTGGAAC TGAGGATGAA |
| | GCTCTGTGGA |
| 251 | AGGACTTAAT TCGACGGATT GGTGAGGTGC GAGGCTATCT |
| | TCGAGAGATC |
| 301 | GAGGAGCTTT GGGCTGCAGA AATTCGTGAG AAAGGGGGCA |
| | ATCTCGAGGA |
| 351 | CTACGCCCTC TGGAATCACC CAGAGACTAC GATTTACAAT |
| | CTTGTTACCG |
| 401 | ATTACGGAAC CGAAGACTCT ATTTATTTGA TTCCTCAAGA |
| | AATCGGAGCG |
| 451 | ATTAAAATCG CAACCTTATC GAAATTTGTA GTTCCTAAAG |
| | AGTCTTTCGA |
| 501 | AGACTGTCTC ACTCAGATCC TATCTCGCTT AGGTATTGGC |
| | GTGCGTCAGG |
| 551 | TCAATTCTTG GATTAAGGAA CTTTATATGA TGCGTAAGGA |
| | GGGCTGCAGT |
| 601 | GTTGCTGGAG TTTTTTCCTC CAGAAAAGAT TTAGAGGCGC |
| | TCCCAGAAAC |
| 651 | AGCCTATATT GGTTTTGTAT TGAATTCGAA CGTAGATGCG |
| | CATACCAATC |
| 701 | AACATGTCTT AAAAAAGTTC ATTAACCCTG AAACAACGCA |
| | TGTAGATGTG |
| 751 | ATTGCAGGAC GTGTGTGGAT TTTTGGTTCT GCGGGGGAAG |
| | TCGGCGAGCT |
| 801 | TCTGAAGATT TATAATTTTG TGCAGTCGGA GAGCATACGT |
| | CAAGAGTATC |
| 851 | GGGTGATTCC CTTAACTAAG ATCGATCCAG GGGAGATGAT |
| | TTCCATTCTC |
| 901 | AACGCAGCAT TTCGTGAGGA TCTGACTAAA GATGTTAGTG |
| | AAGAATCTTT |
| 951 | AGGCCTTCGT GTAGTTCCTT TACAGTATCA AGGGCGTTCG |
| | TTGTTTTTAA |
| 1001 | GTGGAACCGC GGCGTTAGTG CAGCAAGCGC TGACTCTCAT |
| | TCGAGAGCTT |
| 1051 | GAAGAAGGGA TTGAGAACCC TACGGATAAA ACAGTATTTT |
| | GGTATAACGT |
| 1101 | CAAGCACTCC GATCCCCAAG AGTTGGCGGC ATTGCTTTCC |
| | CAAGTCCATG |
| 1151 | ATGTCTTCTC TGGCGAGAAT AAGGCGAGTG TCGGAGCTGC |
| | AGATGGATGT |
| 1201 | GGGTCGCAAT TAAATGCCTC GATCCAAATT GATACTACAG |
| | TAAGTTCTTC |
| 1251 | TGCGAAAGAT GGCTCAGTGA AGTACGGAAA CTTCATCGCG |
| | GATTCTAAGA |
| 1301 | CAGGAACTCT GATTATGGTG GTTGAGAAAG AAGTTCTTCC |
| | ACGTATTCAG |
| 1351 | ATGCTACTTA AGAAACTAGA TGTCCCTAAA AAGATGGTCC |
| | GTATCGAGGT |
| 1401 | GCTGTTATTT GAAAGAAAAT TGGCACATGA GCAGAAATCT |
| | GGGTTAAATC |
| 1451 | TTCTACGTCT TGGTGAGGAA GTTTGTAAAA AAGGGTGCAG |
| | TCCTTCTGTG |
| 1501 | TCTTGGGCCG GGGGTACTGG CATACTAGAA TTTTTATTTA |
| | AAGGAAGTAC |
| 1551 | GGGATCTTCG ATAGTTCCTG GTTATGATCT CGCCTATCAA |
| | TTTTTAATGG |
| 1601 | CTCAAGAGGA CGTTCGGATT AATGCGAGTC CTTCTGTAGT |
| | TACTATGAAC |
| 1651 | CAAACCCCAG CACGGATTGC TGTTGTTGAT GAAATGTCAA |
| | TAGCGGTGTC |
| 1701 | TTCAGATAAA GATAAAGCGC AATACAATCG TGCGCAGTAC |
| | GCTATCATGA |
| 1751 | TAAAAATGCT CCCCGTAATT AATGTGGGAG AGGAAGACGG |
| | AAAAAGTTAC |
| 1801 | ATTACTTTAG AGACAGACAT CACCTTTGAT ACTACGGGAA |
| | AAAATCATGA |
| 1851 | TGATCGTCCT GATGTTACAA GGCGTAATAT TACTAATAAG |
| | GTGCGCATTG |
| 1901 | CTGACGGAGA GACTGTGATT ATTGGAGGTT TGCGTTGCAA |
| | ACAGATGTCA |
| 1951 | GATTCTCATG ATGGCATTCC TTTCCTTGGA GACATTCCTG |
| | GTATAGGGAA |
| 2001 | GTTATTTGGA ATGAGTTCCA CATCAGACAG TCTCACGGAG |
| | ATGTTTGTAT |
| 2051 | TTATCACTCC GAAGATCCTA GAAAATCCTG TAGAGCAACA |
| | AGAACGTAAA |
| 2101 | GAAGAAGCTT TACTCTCTTC GCGCCCTGGA GAGAGAGAAG |
| | AATACTATCA |
| 2151 | GGCTTTAGCA GCTAGTGAGG CTGCAGCACG AGCAGCTCAT |
| | AAAAAATTAG |
| 2201 | AGATGTTCCC GGCATCAGGA GTATCTTTAT CTCAGGTAGA |
| | GAGGCAAGAA |
| 2251 | TACGATGGCT GCTAG |

The PSORT algorithm predicts periplasmic (0.920).

The protein was expressed in E. coli and purified as a GST-fusion product (FIG. 82A) and also in his-tagged form. The recombinant proteins were used to immunise mice, whose sera were used in a Western blot (FIG. 82B) and for FACS analysis.

These experiments show that cp7127 is a surface-exposed and immunoaccessible protein, and that it is a useful immunogen. These properties are not evident from the sequence alone.

### Example 83

The following C. pneumoniae protein (PID 4377133) was expressed <SEQ ID 165; cp7133>:

| | |
|---|---|
| 1 | MQPFIFTLLC LPSLVSLVAF DAANAKRCA CAQTIERGEN |
| | FFSIKRSACA |
| 51 | EIEYQEKSRH ASAIERISKD KGKVTPKQIA KVATKKKQRY |
| | RLLQVPFSRP |
| 101 | PNNSRYNLYA LLSEPPECYS DTASWYAIFI RLLRRAYVDT |
| | GNVPPGSEYA |
| 151 | IANALISNKQ EILERGAQLG PDVIETLTLP EEQABIFYKM |
| | LKGSSNSQSL |
| 201 | LNFLHYEEKS LGHCKLNLIF MDPLLLEAVL DHPDAYRETS |
| | LLRDGIWEAV |
| 251 | KRQEHAIQEH GQAAALELFK TRTDFRLELR DKMQLLLSRY |
| | DLTPLLNKKM |
| 301 | FDYTLGSAGD YLFLVDPDTX AISRCRCPSK SIKL |

A predicted signal peptide is highlighted.

The cp7133 nucleotide sequence <SEQ ID 166> is:

| | |
|---|---|
| 1 | ATGCAACCTT TTATCTTTAC TTTACTGTGC TTGACATCTT |
| | TGGTTTCTTT |
| 51 | AGTCGCCTTT GATGCTGCGA ATGCTCGTAA ACGTTGTGCC |
| | TGTGCTCAAA |
| 101 | CTATAGAACG TGGAGAGAAC TTCTTTTCCA TAAAACGCTC |
| | TGCTTGTGCT |
| 151 | GAAATCGAAT ATCAAGAAAA ATCTCGCCAC GCCTCAGCAA |
| | TTGAAAGAAT |
| 201 | CTCAAAAGAT AAAGGCAAAG TCACTCCAAA GCAGATTGCG |
| | AAAGTAGCTA |
| 251 | CTAAGAAAAA GCAAAGATAC CGTTTATTGC AGGTTCCTTT |
| | TTCAAGGCCT |
| 301 | CCGAATAACT CAAGGTATAA CCTCTATGCT TTGCTTAGTG |
| | AACCTCCCGA |
| 351 | ATGCTATAGC GATACAGCAT CATGGTATGC TAPTTTTATT |
| | CGGTTACTPC |
| 401 | GACGTGCTTA TGTAGACACG GGAAATGTAC CTCCTGGATC |
| | TGAGTATGCC |
| 451 | ATCGCTAATG CTTTGATAAG TAACAAACAA GAGATTTTAG |
| | AGAGGGGAGC |
| 501 | GCAGCTTGGA CCCGATGTTA TTGAAACTCT AACATTGCCT |
| | GAGGAACAAG |
| 551 | CCGAGATTTT TTATAAAATG CTCAAAGGGT CGTCAAACTC |
| | TCAGTCGCTA |
| 601 | CTGAATTTTC TGCATTATGA AGAGAAAAGC TTAGGCCAGT |
| | GTAAGCTAAA |
| 651 | TCTGATCTTC ATGGATCCCC TACTGTTAGA AGCTGTTCTA |
| | GATCATCCCG |
| 701 | ATGCTTATAG GGAAACGTCG CTCCTGCGCG ATGGCATTTG |
| | GGAAGCGGTG |
| 751 | AAGCGTCAAG AACATGCCAT CCAAGAACAT GOCCAGGOAG |
| | CTGCTTTGGA |
| 801 | GCTTTTTAAA ACACGCACCG ACTTCCGCCT GGAGCTGCGA |
| | GATAAGATGC |
| 851 | AGTTACTTCT AAGTCGATAC GATTTGCTCC CCTTATTAAA |
| | TAAAAAAATG |
| 901 | TTCGACTACA CCTTAGGAAG TGCCGGAGAT TACTTATTTT |
| | TGGTAGACCC |
| 951 | AGATACTAAG GCAATTTCTC GATGTCGCTG CCCTTCAAAG |
| | AGTATTAAAT |
| 1001 | TATAA |

The PSORT algorithm predicts outer membrane (0.92).

The protein was expressed in E. coli and purified as a GST-fusion product (FIG. 83A) and also in his-tagged form. The recombinant proteins were used to immunise mice, whose sera were used in a Western blot (FIG. 83B) and for FACS analysis.

These experiments show that cp7133 is a surface-exposed and immunoaccessible protein, and that it is a useful immunogen. These properties are not evident from the sequence alone.

### Example 84

The following C. pneumoniae protein (PID 4377222) was expressed <SEQ ID 167; cp7222>:

| | |
|---|---|
| 1 | MNRRDMVITA VVVNAILLVA LFVTSKRIGV KDYDEGFRNF |
| | ASSKVTQAVV |
| 51 | SEEKVIEKPV VAEVPSRPIA KETLAAQFIE SKPVIVTTPP |
| | VPVVSETPEV |
| 101 | PTVAVPPQPV RBTVKEEQAP YATVVVKKGD FLBRIARANH |
| | TTVAKLMQIN |
| 151 | DLTTTQLIKG QVIKVPTSQD VSNEKTPQTQ TANPENYYIV |
| | QEGDSPWTIA |
| 201 | LRNHIRLDDL LKMNDLDEYK ARRLKPGDQL RIR* |

A predicted signal peptide is highlighted.

The cp7222 nucleotide sequence <SEQ ID 168> is:

| | |
|---|---|
| 1 | ATGAATCGTA GAGACATGGT AATAACAGCT GTCGTAGTGA |
| | ATGCTATATT |
| 51 | GCTTGTGGCT CTTTTCGTCA CATCAAAGCG TATTGGCGTC |
| | AAGGACTATC |
| 101 | ACGAGGGATT CCGTAATTTT GCTTCTAGCA AGGTTACACA |
| | AGCAGTAGTT |
| 151 | TCAGAAGAAA AAGTCATAGA AAAGCCTGTA GTCGCAGAAG |
| | TGCCTAGCCG |
| 201 | TCCTATCGCT AAAGAGACTC TAGCTGCACA GTTTATTGAA |
| | AGTAAGCCGG |
| 251 | TTATTGTAAC CACACCACCC GTGCCTGTTG TTAGCGAAAC |
| | CCCAGAAGTG |
| 301 | CCTACTGTGG CAGTTCCCCC TCAGCCTGTT CGTGAGACAG |
| | TAAAAGAGGA |
| 351 | ACAAGCTCCT TATGCTACTG TTGTAGTGAA AAAAGGAGAT |
| | TTTCTCGAAC |
| 401 | GCATTGCGAG AGCAAATCAT ACTACCGTTG CAAAATTGAT |
| | GCAGATCAAT |
| 451 | GATCTTACCA CCACCCAACT TAAAATTGGT CAGGTCATCA |
| | AAGTCCCTAC |
| 501 | GTCTCAAGAT GTCAGCAACG AAAAAACTCC TCAAACACAG |
| | ACCGCAAACC |
| 551 | CTGAAAATTA TTATATCGTC CAAGAAGGGG ATAGCCCGTG |
| | GACAATAGCA |
| 601 | TTGCGTAACC ATATTCGATT GGATGATTTG CTAAAAATGA |
| | ATGATCTCGA |
| 651 | TGAATATAAA GCCCGGCGCC TTAAGCCTGG AGATCAGTTG |
| | CGCATACGTT |
| 701 | GA |

The PSORT algorithm predicts periplasmic (0.935).

The protein was expressed in E. coli and purified as a GST-fusion product (FIG. 84A) and also in his-tagged form. The recombinant proteins were used to immunise mice, whose sera were used in a Western blot (FIG. 84B) and for FACS analysis.

These experiments show that cp7222 is a surface-exposed and immunoaccessible protein, and that it is a useful immunogen. These properties are not evident from the sequence alone.

### Example 85

The following C. pneumoniae protein (PID 4377225) was expressed <SEQ ID 169; cp7225>:

| | |
|---|---|
| 1 | MKGTPQYHFI GIGGIGMSAL AHILLDRGYE VSGSDLYESY TIESLKAKGA |
| 51 | RCFSGHDSSH VPHDAVVVYS SSIAPDNVEY LTAIQRSSRL LHRAELLSQL |
| 101 | MEGYESILVS GSHGKTGTSS LIRAIFQEAQ KDPSYAIGGL AANCLNGYSG |
| 151 | SSKIFVAEAD ESDGSLKHYT PRAVVITNID NEHLNNYAGN LDNLVQVIQD |
| 201 | FSRKVTDLNK VFYNGDCPIL KGNVQGISYG YSPECQLHIV SYNQKAWQSH |
| 251 | FSFTFLGQEY QDIELNLPGQ HNAANAAAAC GVALTFGIDI NIIRKALKKF |
| 301 | SGVHRRLERK NISESFLFLE DYAHHPVEVA HTLRSVRDAV GLRRVIAIFQ |
| 351 | PHRFSRLEEC LQTFPKAFQE ADEVILTDVY SAGESPRESI ILSDLAEQIR |
| 401 | KSSYVHCCYV PHGDIVDYLR NYIRIHDVCV SLGAGNIYTI GEALKDWNPK |
| 451 | KLSIGLVCGG KSCEHDISLL SAQHVSKYIS PEFYDVSYFI INRQGLWRTG |
| 501 | KDFPHLIEET QGDSPLSSEI ASALAKVDCL FPVLHGPFGE DGTIQGFFEI |
| 551 | LGKPYAGPSL SLAATAMDKL LTKRIASAVG VPVVPYQPLN LCFWKRNPEL |
| 601 | CIQNLIETFS FPMIVKTAHL GSSIGIFLVR DKEELQEKIS EAFLYDTDVF |
| 651 | VEESRLGSRE IEVSCIGHSS SWYCMAGPNE RCGASGFIDY QEKYGFDGID |
| 701 | CAKISFDLQL SQESLDCVRE LAERVYRAMQ GKGSARIDFF LDEEGNYWLS |
| 751 | EVNPIPGMTA ASPFLQAFVH AGWTQEQIVD HFIIDALHKF DKQQTIEQAF |
| 801 | TKEQDLVKR* |

The cp7225 nucleotide sequence <SEQ ID 170> is:

| | |
|---|---|
| 1 | ATGAAGGGAA CTCCTCAGTA TCATTTTATC GGTATCGGTG GTATAGGAAT |
| 51 | GAGCGCTTTA GCTCATATTT TGCTTGATCG TGGCTATGAG GTCTCTGGAA |
| 101 | GCGACTTATA TGAAAGCTAT ACGATCGAAA GCCTGAAAGC TAAAGGTGCG |
| 151 | AGGTGTTTCT CAGGCCATGA TTCCTCCCAT GTTCCTCATG ATGCCGTCGT |
| 201 | TGTTTATAGC TCAAGTATAG CCCCTGATAA TGTAGAGTAT CTTACCGCTA |
| 251 | TTCAAAGATC ATCACGTCTT CTTCATAGAG CAGAGCTCTT GAGTCAGCTT |
| 301 | ATGGAGGGTT ATGAAAGCAT TCTGGTTTCA GGAAGCCATG GGAAGACAGG |
| 351 | GACCTCATCT CTAATTCGAG CGATTTTCCA GGAAGCTCAG AAAGATCCCT |
| 401 | CCTATGCTAT TGGAGGACTC GCTGCAAACT GCCTGAATGG GTATTCTGGA |
| 451 | TCATCGAAAA TCTTCGTTGC CGAAGCCGAT GAAAGTGATG GGTCTTTAAA |
| 501 | GCACTACACT CCCCGTGCAG TAGTCATTAC AAATATAGAT AATGAACATT |
| 551 | TGAATAATTA CGCTGGGAAT CTTGATAACC TGGTTCAGGT AATCCAGGAC |
| 601 | TTCTCTAGAA AAGTAACAGA TCTCAATAAG GTATTCTATA ACGGGGATTG |
| 651 | TCCTATTTTG AAAGGAAATG TCCAAGGGAT TTCTTATGGA TATTCACCAG |
| 701 | AATGTCAATT GCATATCGTT TCCTATAATC AAAAGGCATG GCAATCTCAC |
| 751 | TTTTCCTTTA CCTTTTTAGG CCAGGAGTAT CAACACATTG AGCTCAATCT |
| 801 | CCCTGGACAA CATAACGCTG CAAATGCAGC AGCAGCCTGT GGAGTTGCTC |
| 851 | TTACCTTTGG CATAGACATA AACATCATTC GAAAAGCTCT CAAAAAATTC |
| 901 | TCGGGAGTTC ATCGACGTCT AGAAAGAAAA AATATATCCG AAAGCTTTCT |
| 951 | TTTCTTAGAA GATTATGCTC ATCATCCTGT AGAGGTTGCA CATACCCTGC |
| 1001 | GCTCTGTGCG TGATGCTGTG GGTTTGCGAA GAGTCATCGC AATTTTTCAA |
| 1051 | CCACATCGAT TCTCTCGTTT AGAAGAGTGC TTACAAACCT TCCCCAAAGC |
| 1101 | TTTCCAAGAA GCTGATGAAG TCATACTTAC AGATGTCTAT AGTGCCGGAG |
| 1151 | AAAGTCCTAG AGAGTCTATC ATTCTTTCCG ACCTTGCGGA ACAGATTCGT |
| 1201 | AAGTCTTCTT ATGTCCATTG TTGTTATGTT CCCCATGGAG ACATCGTAGA |
| 1251 | TTATCTACGA AACTACATTC GCATTCATGA TGTCTGTGTT TCTCTAGGAG |
| 1301 | CTGGAAATAT CTATACTATT GGAGAGGCTT TAAAAGACTT TAACCCTAAA |
| 1351 | AAATTATCCA TAGGACTCGT CTGTGGAGGG AAATCTTGCG AACACGATAT |
| 1401 | TTCTCTACTT TCTGCTCAAC ATGTCTCTAA ATATATTTCT CCTGAATTCT |
| 1451 | ATGATGTGAG TTACTTCATC ATAAATCGTC AGGGCTTATG GAGAACAGGA |
| 1501 | AAGGATTTTC CTCATCTTAT TGAAGAGACT CAAGGGGATT CGCCACTTTC |
| 1551 | TTCTGAAATC GCTTCAGCTT TAGCAAAAGT CGACTGTTTG TTTCCCGTGC |
| 1601 | TCCATGGCCC ATTTGGAGAG GATGGTACGA TCCAGGGATT TTTTGAAATC |
| 1651 | TTAGGAAAAC CTTATGCCGG ACCCTCACTA TCTTTAGCAG CAACTGCAAT |
| 1701 | GGATAAGCTG TTAACAAAAC GAATTGCATC AGCAGTGGGT GTTCCTGTAG |
| 1751 | TCCCTTACCA ACCTTTAAAT CTCTGTTTCT GGAAACGCAA TCCAGAACTA |
| 1801 | TGTATTCAGA ATCTTATAGA GACATTTTCT TTCCCTATGA TTGTAAAAAC |
| 1851 | TGCACATTTG GGATCTAGTA TTGGGATATT TTTAGTCCGT GATAAAGAGG |
| 1901 | AATTACAAGA AAAGATCTCA GAAGCATTTC TATATGACAC GGATGTGTTT |
| 1951 | GTGGAGGAAA GTCGCTTAGG GTCTCGTGAA ATCGAAGTGT CCTGTATCGG |
| 2001 | CCATTCTTCT AGCTGGTATT GTATGGCAGG GCCTAATGAA CGCTGTGGTG |
| 2051 | CTAGTGGGTT TATTOATTAT CAAGAGAAAT ATGGATTTGA TGGCATAGAT |
| 2101 | TGCGCAAAGA TCTCTTTTGA TTTACAGCTC TCACAAGAAT CTTTAGATTG |
| 2151 | TGTTAGAGAA CTTGCAGAGC GTGTCTACCG AGCAATGCAA GGAAAAGGTT |
| 2201 | CAGCTCGAAT AGATTTTTTC TTGGATGAAG AGGGGAATTA TTGGTTGTCA |
| 2251 | GAGGTCAATC CTATTCCAGG AATGACAGCA GCTAGCCCAT TTTTACAAGC |
| 2301 | TTTTGTTCAC GCAGGATGGA CGCAAGAACA AATTGTAGAT CACTTTATTA |
| 2351 | TAGATGCTCT ACATAAGTTT GATAAGCAGC AGACTATCGA ACAGGCATTC |
| 2401 | ACTAAAGAAC AAGATTTAGT TAAAAGATAA |

The PSORT algorithm predicts inner membrane (0.16).

The protein was expressed in E. coli and purified as a his-tag product (FIG. 85A). The recombinant protein was used to immunise mice, whose sera were used in a Western blot (FIG. 85B) and for FACS analysis.

These experiments show that cp7225 is a surface-exposed and immunoaccessible protein, and that it is a useful immunogen. These properties are not evident from the sequence alone.

### Example 86

The following C. pneumoniae protein (PID 4377248) was expressed <SEQ ID 171; cp7248>:

| | |
|---|---|
| 1 | MKFWLQGCAF VGCLLLTLPC CAARRRASGE NLQQTRPIAA ANLQWESYAE |
| 51 | ALEHSKQDHK PICLFFTGSD WCMWCIKMQD QILQSSEFKH FAGVHLHMVE |
| 101 | VDFPQKNHQP EEQRQKNQEL KAQYKVTGFP ELVFIDAEGK QLARMGFEPG |
| 151 | GGAAYVSKVK SALKLR* |

A predicted signal peptide is highlighted.

The cp7248 nucleotide sequence <SEQ ID 172> is:

| | |
|---|---|
| 51 | TTTACCTTGT TGTGCTGCAC GAAGACGTGC TTCTGGAGAA AATTTGCAAC |
| 101 | AAACTCGTCC TATAGCAGCT GCAAATCTAC AATGGGAGAG CTATGCAGAA |
| 151 | GCTCTTGAAC ATTCTAAACA AGATCACAAA CCTATTTGTC TTTTCTTTAC |
| 201 | AGGATCAGAC TGGTGTATGT GGTGCATAAA AATGCAAGAC CAGATTTTGC |
| 251 | AAAGCTCTGA GTTTAAGCAT TTTGCGGGTG TGCATCTGCA TATGGTTGAA |
| 301 | GTTGATTTCC CCCAAAAGAA TCATCAACCT GAAGAGCAGC GCCAAAAAAA |
| 351 | TCAAGAACTG AAAGCTCAAT ATAAAGTTAC AGGATTCCCC GAACTGGTCT |
| 401 | TCATAGATGC AGAAGGAAAA CAGCTTGCTC GCATGGGATT TGAGCCTGGT |
| 451 | GGTGGAGCTG CTTACGTAAG CAAGGTGAAG TCTGCTCTTA AACTACGTTA |
| 501 | A |

The PSORT algorithm predicts periplasmic (0.932).

The protein was expressed in E. coli and purified as a GST-fusion product (FIG. 86A) and also in his-tagged form. The recombinant proteins were used to immunise mice, whose sera were used in a Western blot (FIG. 86B) and for FACS analysis.

The cp7248 protein was also identified in the 2D-PAGE experiment.

These experiments show that cp7248 is a surface-exposed and immunoaccessible protein, and that it is a useful immunogen. These properties are not evident from the sequence alone.

### Example 87

The following C. pneumoniae protein (PID 4377249) was expressed <SEQ ID 173; cp7249>:

| | |
|---|---|
| 1 | MIPSPTPINF RDDTILETDP KPSLIMFSSR KTEIASERRK AHPTLFKVLG |
| 51 | TIWNIVKFII SIILFLPLAL LWVLKKTCQF FILPSSIISQ SMSKTAVAIR |
| 101 | RMTPLSHIKQ LLSLKEISAA DRVVIQYDDL VVDSLAIKIP HALPHRWILY |
| 151 | SQGNSGLMEN LFDRGDSSLH QLAKATGSNL LVFNYPGIMS SKGEAKRENL |
| 201 | VKSYQACVRY LRDEETGPKA NQIIAFGYSL GTSVQAAALD REVTDGSDGT |
| 251 | SWIVVKDRGP RSLADVANQI CKPIASAIIK LVGWNIDSVK PSERLRCPEI |
| 301 | FIYNSNHDQE LISDGLFERE NCVATPFLEL PEVKTSGTKI PIPERDLLHL |
| 351 | NPLSPNVVDR LAAVISNYLD SENRKSQQPD * |

The cp7249 nucleotide sequence <SEQ ID 174> is:

| | |
|---|---|
| 1 | ATGATCCCAT CCCCTACCCC AATAAACTTT CGTGATGATA CGATTCTAGA |
| 51 | GACGGATCCA AAGCCGTCTT TAATCATGTT CTCTTCAAAA AAAACAGAGA |
| 101 | TAGCTTCTGA AAGACGGAAG GCCCATCCCA CCTTATTTAA AGTTCTAGGA |
| 151 | ACGATTTGGA ATATTGTGAA GTTTATTATC TCAATCATTC TGTTCCTTCC |
| 201 | CTTAGCGTPA TTGTGGGTAC TCAAGAAAAC CTGTCAGTTT TTCATTCTCC |
| 251 | CATCTTCTAT CATATCTCAG AGCATGTCAA AAACAGCTGT GGCAATTCGG |
| 301 | CGAATGACCT TTCTGTCCCA TATTAAACAA CTCCTAAGCC TTAAGGAAAT |
| 351 | CTCAGCTGCC GATCGTGTGG TTATACAATA TGACGATTTG GTGGTTGATA |
| 401 | GCTTAGCTAT AAAGATACCT CATGCTCTTC CCCACAGGTG GATTCTTTAT |
| 451 | TCTCAAGGAA ACTCTGGATT GATGGAAAAC CTGTTCGATC GGGGCGATTC |
| 501 | CTCTCTACAC CAGCTAGCCA AAGCAACCGG CTCGAATCTT CTTGTGTTCA |
| 551 | ACTATCCTGG AATTATGTCC AGCAAAGGAG AAGCGAAACG AGAAAATCTG |
| 601 | GTTAAATCGT ATCAGGCATG CGTACGCTAC CTACGAGATG AAGAGACAGG |
| 651 | TCCTAAAGCC AATCAAATCA TAGCTTTCGG ATACTCTTTG GGAACTAGTG |
| 701 | TCCAAGCTGC TGCTCTAGAT CGTGAGCTCA CTOATGGCAG TGATGGAACT |
| 751 | TCATGGATTG TTGTAAAAGA TCGGGGCCCT CGCTCTCTAG CAGATGTCGC |
| 801 | GAATCAAATT TGTAAGCCCA TAGCTTCCGC GATTATAAAA CTCGTTGGTT |
| 851 | GGAACATAGA CTCTGTCAAA CCTAGCGAAA GATTGCGTTG TCCCGAAATT |
| 901 | TTCATTTACA ACTCTAATCA TGATCAAGAA CTCATTAGCG ACGGCCTCTT |
| 951 | CGAAAGAGAA AATTGCGTAG CAACACCTTT TCTAGAGCTT CCTGAAGTAA |
| 1001 | AAACCTCGGG GACTAAAATT CCTATACCCG AAAGGGATCT TCTCCATCTA |
| 1051 | AATCCTCTCA GTCCAAATGT AGTAGACAGA TTAGCAGCAG TGATCTCTAA |
| 1101 | TTATTTAGAT TCTGAAAACA GAAAGTCTCA GCAACCTGAT TAA |

The PSORT algorithm predicts inner membrane (0.571).

The protein was expressed in E. coli and purified as a GST-fusion product (FIG. 87A). The recombinant protein was used to immunise mice, whose sera were used in a Western blot (FIG. 87B) and for FACS analysis.

These experiments show that cp7249 is a surface-exposed and immunoaccessible protein, and that it is a useful immunogen. These properties are not evident from the sequence alone.

### Example 88

The following C. pneumoniae protein (PID 4377261) was expressed <SEQ ID 175; cp7261>:

| | |
|---|---|
| 1 | MLPISILLFY VILGCLSAYI ADKKKRNVIG WFFAGAFFGF IGLVVLLLLP |
| 51 | SRRNALEKPQ NDPFDNSDLF DDLKKSLAGN DEIPSSGDLQ EIVIDTEKWF |
| 101 | YLNKDRENVG PISFEELVVL LKGKTYPEEI WVWKKGMKDW QRVKDVPSLQ |
| 151 | QALKEASK* |

The cp7261 nucleotide sequence <SEQ ID 176> is:

| | |
|---|---|
| 1 | ATGCTCCCTA TTTCGATTTT ATTATTTTAT GTGATTCTAG GTTGTCTATC |
| 51 | TGCCTACATA GCAGATAAGA AAAAACGAAA TGTTATTGGC TGGTTTTTTG |
| 101 | CAGGAGCATT TTTTTCATTT ATTGGTCTAG TTGTCCTTCT TCTTCTTCCT |
| 151 | TCTCGTCGAA ACGCTTTAGA AAAGCCACAA AACGATCCTT TTGATAACTC |
| 201 | CGATCTTTTT GATGATTTGA AAAAAAGTTT AGCAGGTAAT GACGAGATAC |
| 251 | CCTCATCGGG AGATCTTCAA GAAATCGTTA TCGATACAGA GAAGTGGTTT |
| 301 | TATTTAAATA AAGATAGAGA AAACGTAGGT CCGATATCTT TTGAGGAGTT |
| 351 | GGTCGTACTT TTAAAGGGAA AAACGTATCC AGAAGAAATT TGGGTATGGA |
| 401 | AAAAGGGAAT GAAAGATTGG CAACGAGTGA AGGATGTTCC ATCACTACAA |
| 451 | CAGGCTTTGA AAGAAGCATC AAAATAA |

The PSORT algorithm predicts inner membrane (0.848).

The protein was expressed in E. coli and purified as a GST-fusion product (FIG. 88A). The recombinant protein was used to immunise mice, whose sera were used in a Western blot (FIG. 88B) and for FACS analysis.

These experiments show that cp7261 is a surface-exposed and immunoaccessible protein, and that it is a useful immunogen. These properties are not evident from the sequence alone.

### Example 89

The following C. pneumoniae protein (PID 4377305) was expressed <SEQ ID 177; cp7305>:

| | |
|---|---|
| 1 | MEVYSFHPAV RTSFQHRVMA ALDAWFFLGG HRLKVVSLDS CNSGWAYQEL |
| 51 | VSISTTEKVL KLLSYLLVPI VIIALLIRCL LHSNFRIDVE KERWLKIREL |
| 101 | GIDIESCKLP SSYVNQVSSF IWFEKDKSKR PRIDVDYHTL HSKDWVVFPI |
| 151 | VFQKIPKTSR FSYWFSQKET RKRDYVRNML DHVIGYLTSE GGEWLQYISK |
| 201 | TSYQSATSLD PERVLQYCLT DNQELQGEVQ RLLNEESATK SSGDKEVLLS |
| 251 | HVSDIICQCW WPKFLEVIQS PAFIEELVEE VSGKLNLDFL CLEKANTLDQ |
| 301 | ELRNSLLRAV VHHGSEGVDI KKVGAGLIIY TEAIQLQIPF SRS* |

The cp7305 nucleotide sequence <SEQ ID 178> is:

| | |
|---|---|
| 1 | ATGGAAGTTT ATAGTTTTCA CCCTGCGGTA AGGACTTCGT TTCAGCACCG |
| 51 | TGTAATGGCA GCACTAGATG CTTGGTTTTT TCTAGGAGGG CACCGTTTAA |
| 101 | AAGTAGTTTC TCTAGATAGT TGTAACTCAG GTTGGGCGTA TCAAGAACTT |
| 151 | GTGTCTATTT CAACGACAGA AAAAGTCTTG AAACTACTCT CTTACCTACT |
| 201 | CGTACCGATT GTCATAATAG CTCTGTTAAT TCGTTGTCTT TTACATAGCA |
| 251 | ATTTTAGGAT AGACGTAGAG AAGGAACGTT GGTTAAAAAT AAGGGAGTTA |
| 301 | GGAATTGATA TAGAAAGCTG CAAACTCCCC AGTTCTTATG TAAACCAGGT |
| 351 | TTCCTCGTTT ATTTGGTTTG AAAAAGATAA ATCCAAACGG CCACGTATTG |
| 401 | ATCTAGATTA TCATACGCTA CATAGCAAAG ACTGGGTAGT TTTCCCTATC |
| 451 | GTTTTTCAGA AAATTCCAAA GACCTCGCGT TTCAGTTATT GGTTCTCACA |
| 501 | AAAAGAAACA AGGAAGACGG ATTATGTGAG AAATATGCTG GACCACGTCA |
| 551 | TTGGTTATCT AACGTCAGAA GGTGGGGAGT GGTTGCAGTA TATATCGAAA |
| 601 | ACCTCTTATC AAAGCGCTAC TTCCTTGGAT CCTGAAAGAG TTCTTCAATA |
| 651 | TTGCTTAACT GATAACCAGG AGCTCCAGGG AGAAGTGCAA CGTTTGCTTA |
| 701 | ATGAGGAGAG TGCGACCAAA AGCTCTGGGG ATAAGGAAGT TTTGTTAAGT |
| 751 | CATGTATCTG ACATTATTTG CCAGTGTTGG TGGCCAAAGT TTCTTGAAGT |
| 801 | TATACAATCT CCGGCCTTTA TTGAAGAATT AGTAGAAGAA GTGAGTGGTA |
| 851 | AACTTAATTT AGATTTTTTA TGCCTAGAAA AGGCTAATAC ATTAGATCAG |
| 901 | GAGTTGAGAA ACAGTCTTCT AAGAGCAGTC GTACACCACG GTTCTGAAGG |
| 951 | AGTTGATATT AAGAAAGTTG GTGCCGGCCT CATTATTTAT ACGGAAGCTA |
| 1001 | TTCAATTACA GATTCCCTTC TCAAGGAGTT AA |

The PSORT algorithm predicts inner membrane (0.508).

The protein was expressed in E. coli and purified as a GST-fusion product (FIG. 89A) and also as a double GST/his fusion. The recombinant proteins were used to immunise mice, whose sera were used in a Western blot (FIG. 89B) and for FACS analysis.

These experiments show that cp7305 is a surface-exposed and immunoaccessible protein, and that it is a useful immunogen. These properties are not evident from the sequence alone.

### Example 90

The following C. pneumoniae protein (PID 4377347) was expressed <SEQ ID 179; cp7347>:

| | |
|---|---|
| 1 | MKKGKLGAIV FGLLFTSSVA GFSKDLTKDN AYQDLNVIEH LISLKYAPLP |
| 51 | WKELLFGWDL SQQTQQARLQ LVLEEKPTTN YCQKVLSNYV RSLNDYHAGI |
| 101 | TFYRTESAYI PYVLKLSEDG HVFVVDVQTS QGDIYLGDEI LEVDGMGIRE |
| 151 | AIESLRFGRG SATDYSAAVR SLTSRSAAFG DAVPSGIAML KLRRPSGLIR |
| 201 | STPVRWRYTP EHIGDFSLVA PLIPEHKPQL PTQSCVLFRS GVNSQSSSSS |
| 251 | LFSSYMVPYF WEELRVQNKQ RFDSNHHIGS RNGFLPTFGP ILWEQDKGPY |
| 301 | RSYIFKAKDS QGNPHRIGFL RISSYVWTDL EGLEEDHKDS PWELFGEIID |
| 351 | HLEKETDALI IDQTHNPGGS VFYLYSLLSM LTDHPLDTPK HRMIFTQDEV |
| 401 | SSALHWQDLL EDVFTDEQAV AVLGETMEGY CMDMHAVASL QNFSQSVLSS |
| 451 | WVSGDINLSK PMPLLGFAQV RPHPKHQYTK PLFMLIDEDD FSCGDLAPAI |
| 501 | LKDNGRATLI GKPTAGAGGF VFQVTFPNRS GIKGLSLTGS LAVRKDGEFI |
| 551 | ENLGVAPHID LGFTSRDLQT SRFTDYVEAV KTIVLTSLSE NAKKSEEQTS |
| 601 | PQETPEVIRV SYPTTTSAS* |

A predicted signal peptide is highlighted.

The cp7347 nucleotide sequence <SEQ ID 180> is:

| | |
|---|---|
| 1 | ATGAAAAAAG GGAAATTAGG AGCCATAGTT TTTGGCCTTC TATTTACAAG |
| 51 | TAGTGTTGCT GGTTTTTCTA AGGATTTGAC TAAAGACAAC GCTTATCAAG |
| 101 | ATTTAAATGT CATAGAGCAT TTAATATCGT TAAAATATGC TCCTTTACCA |
| 151 | TGGAAGGAAC TATTATTTGG TTGGGATTTA TCTCAGCAAA CACAGCAAGC |
| 201 | TCGCTTGCAA CTGGTCTTAG AAGAAAAACC AACAACCAAC TACTGCCAGA |
| 251 | AGGTACTCTC TAACTACGTG AGATCATTAA ACGATTATCA TGCAGGGATT |
| 301 | ACGTTTTATC GTACTGAAAG TGCGTATATC CCTTACGTAT TGAAGTTAAG |
| 351 | TGAAGATGGT CATGTCTTTG TAGTCGACGT ACAGACTAGC CAAGGGGATA |
| 401 | TTTACTTAGG GGATGAAATC CTTGAAGTAG ATGGAATGGG GATTCGTGAG |
| 451 | GCTATCGAAA GCCTTCGCTT TGGACGAGGG AGTGCCACAG ACTATTCTGC |
| 501 | TGCAGTTCGT TCCTTGACAT CGCGTTCCGC CGCTTTTGGA GATGCGGTTC |
| 551 | CTTCAGGAAT TGCCATGTTG AAACTTCGCC GACCCAGTGG TTTGATCCGT |
| 601 | TCGACACCGG TCCGTTGGCG TTATACTCCA GAGCATATCG GAGATTTTTC |
| 651 | TTTAGTTGCT CCTTTGATTG CTGAACATAA ACCTCAATTA CCTACACAAA |
| 701 | GTTGTGTGCT ATTCCGTTCC GGGGTAAATT CACAGTCTTC TAGTAGCTCT |
| 751 | TTATTCAGTT CCTACATGGT GCCTTATTTC TGGGAAGAAT TGCGGGTTCA |
| 801 | AAATAAGCAG CGTTTTGACA GTAATCACCA TATAGGGAGC CGTAATGGAT |
| 851 | TTTTACCTAC GTTTGGTCCT ATTCTTTGGG AACAAGACAA GGGGCCCTAT |
| 901 | CGTTCCTATA TCTTTAAAGC AAAAGATTCT CAGGGCAATC CCCATCGCAT |
| 951 | AGGATTTTTA AGAATTTCTT CTTATGTTTG GACTGATTTA GAAGGACTTG |
| 1001 | AAGAGGATCA TAAGGATAGT CCTTGGGAGC TCTTTGGAGA GATCATCGAT |
| 1051 | CATTTGGAAA AAGAGACTGA TGCTTTGATT ATTGATCAGA CCCATAATCC |
| 1101 | TGGAGGCAGT GTTTTCTATC TCTATTCGTT ACTATCTATG TTAACAGATC |
| 1151 | ATCCTTTAGA TACTCCTAAA CATAGAATGA TTTTCACTCA GGATGAAGTC |
| 1201 | AGCTCCGCTT TGCACTGGCA AGATCTACTA GAAGATGTCT TCACAGATGA |
| 1251 | GCAGGCAGTT GCCGTGCTAG GGGAAACTAT GGAAGGATAT TGCATGGATA |
| 1301 | TGCATGCTGT AGCCTCTCTT CAAAACTTCT CTCAGAQTGT CCTTTCTTCC |
| 1351 | TGGGTTTCAG GTGATATTAA CCTTTCAAAA CCTATGCCTT TGCTAGGATT |
| 1401 | TGCACAGGTT CGACCTCATC CTAAACATCA ATATACTAAA CCTTTGTTTA |
| 1451 | TGTTGATAGA CGAGGATGAC TTCTCTTGTG GAGATTTAGC GCCTGCAATT |
| 1501 | TTGAAGGATA ATGGCCGCGC TACTCTCATT GGAAAGCCAA CAGCAGGAGC |
| 1551 | TGGAGGTTTT GTATTCCAAG TCACTTTCCC TAACCGTTCT GGAATTAAAG |
| 1601 | GTCTTTCTTT AACAGGATCT TTAGCTGTTA GGAAAGATGG TGAGTTTATT |
| 1651 | GAAAACTTAG GAGTGGCTCC TCATATTGAT TTAGGATTTA CCTCCAGGGA |
| 1701 | TTTGCAAACT TCCAGGTTTA CTGATTACGT TGAGGCAGTG AAAACTATAG |
| 1751 | TTTTAACTTC TTTGTCTGAG AACGCTAAGA AGAGTGAAGA GCAGACTTCT |
| 1801 | CCGCAAGAGA CGCCTGAAGT TATTCGAGTC TCTTATCCCA CAACGACTTC |
| 1851 | TGCTTCGTAA |

The PSORT algorithm predicts periplasmic space (0.2497).

The protein was expressed in E. coli and purified as a GST-fusion product (FIG. 90A) and also in his-tagged form. The recombinant proteins were used to immunise nice, whose sera were used in a Western blot (FIG. 90B) and for FACS analysis.

These experiments show that cp7347 is a surface-exposed and immunoaccessible protein, and that it is a useful immunogen. These properties are not evident from the sequence alone.

### Example 91

The following C. pneumoniae protein (PID 4377353) was expressed <SEQ ID 181; cp7353>:

| | |
|---|---|
| 1 | MNMPVPSAVP SANITLKEDS STVSTASGIL KTATGEVLVS CTALEGSSST |
| 51 | DALISLALGQ IILATQQELL LQSTNVHQLL FLPPEVVELE IQVVDLLVQL |
| 101 | EHAETITSEP QETQTQSRSE QTLPQQSSSK QSALSPRSLK PEISDSKQQQ |
| 151 | ALQTPKDSAV RKHSEAPSPE TQARASLSQA SSSSQRSLPP QESAPERTLL |
| 201 | EQQKASSFSP LSQFSAEKQK EALTTSRSHE LYKERDQDRQ QREQHDRKHD |
| 251 | QEEDAESKKK KKKRGLQVEA VAEEPGENLD IAALIFSDQM RPPAEETSKK |
| 301 | ETTFKKKLPS PMSVFSRFIP SKNPLSVGSS IHGPIQTPKV ENVFLRFMKL |
| 351 | MARILGQAEA EANELYMRVK QRTDDVDTLT VLISKINNEK KDIDWSENEE |
| 401 | MKALLNRAKE IGVTIDKEKY TWTEEEKRLL KENVQMRKEN MEKITQMERT |
| 451 | DMQRHLQEIS QCHQARSNVL KLLKELMDTF IYNLRP* |

The cp7353 nucleotide sequence <SEQ ID 182> is:

| | |
|---|---|
| 1 | ATGAATATGC CTGTTCCTTC TGCAGTTCCC TCTGCAAATA TAACTCTAAA |
| 51 | AGAAGACAGC TCAACAGTTT CCACAGCCTC TGGAATATTA AAGACTGCAA |
| 101 | CAGGTGAAGT CTTAGTCTCT TGTACAGCGC TAGAAGGAAG CTCTTCTACA |
| 151 | GATGCTTTAA TTAGCTTAGC TTTAGGACAA ATCATTCTTG CGACCCAACA |
| 201 | AGAACTGCTC TTACAAAGCA CAAATGTTCA TCAACTCCTC TTCCTCCCTC |
| 251 | CTGAAGTTGT AGAATTAGAA ATCCAAGTTG TTGACTTGCT AGTGCAATTG |
| 301 | GAACATGCAG AGACAATCAC AAGTGAACCA CAAGAAACAC AAACGCAAAG |
| 351 | TAGGAGTGAG CAGACCCTCC CTCAACAAAG CAGCAGTAAA CAATCTGCTC |
| 401 | TCTCCCCACG CTCCTTAAAA CCTGAAATTT CTGATTCTAA ACAACAGCAA |
| 451 | GCTCTTCAAA CACCAAAAGA CTCTGCTGTA AGAAAACACA GCGAAGCACC |
| 501 | GTCACCTGAG ACACAAGCTC GCGCTTCCTT ATCTCAGGCA AGCTCAAGTT |
| 551 | CTCAGAGATC CTTACCTCCG CAAGAAAGTG CGCCAGAAAG AACACTATTA |
| 601 | GAACAACAAA AAGCAAGCTC CTTCTCTCCT CTATCCCAGT TCTCTGCAGA |
| 651 | GAAACAAAAA GAGGCCCTGA CGACCTCAAA ATCTCATGAA CTCTATAAAG |
| 701 | AACGCGATCA AGATCGCCAA CAAAGAGAGC AGCACGACAG AAAGCACGAT |
| 751 | CAGGAAGAAG ACGCTGAATC TAAAAAGAAA AAGAAGAAAC GTGGTCTCGG |
| 801 | TGTAGAGGCA GTCGCTGAGG AACCCGGAGA AAATCTAGAT ATTGCCGCTT |
| 851 | TAATCTTCTC AGATCAAATG CGACCTCCTG CTGAAGAAAC TTCTAAAAAA |
| 901 | GAAACGACAT TCAAAAAGAA GCTACCTTCT CCAATGTCTG TGTTTAGCAG |
| 951 | ATTCATCCCT AGTAAGAATC CGTTATCTGT AGGCTCTTCA ATACACGGGC |
| 1001 | CTATACAAAC TCCAAAAGTA GAAAATGTGT TCTTAAGGTT CATGAAGCTC |
| 1051 | ATGGCAAGAA TCTTAGGCCA AGCCGAAGCC GAAGCTAATG AACTCTACAT |
| 1101 | GCGAGTCAAA CAACGTACCG ATGATGTAGA CACACTCACA GTCCTTATCT |
| 1151 | CTAAGATCAA TAATGAAAAG AAAGACATTG ATTGGAGTGA AAATGAAGAG |
| 1201 | ATGAAAGCTC TTTTAAATCG AGCTAAAGAG ATTGGAGTCA CTATAGACAA |
| 1251 | AGAAAAATAT ACTTGGACAG AAGAGGAAAA AAGACTTCTA AAAGAGAATG |
| 1301 | TCCAAATGCG CAAAGAGAAT ATGGAGAAAA TCACTCAAAT GGAAAGGACG |
| 1351 | GACATGCAAA GGCACCTCCA AGAGATTTCT CAATGTCATC AAGCGCGCTC |
| 1401 | TAATGTATTG AAGTTATTGA AAGAACTTAT GGACACCTTC ATTTACAACC |
| 1451 | TACGCCCCTA A |

The PSORT algorithm predicts cytoplasm (0.1308).

The protein was expressed in E. coli and purified as a GST-fusion product (FIG. 91A). The recombinant protein was used to immunise mice, whose sera were used in a Western blot (FIG. 91B) and for FACS analysis.

These experiments show that cp7353 is a surface-exposed and immunoaccessible protein, and that it is a useful immunogen. These properties are not evident from the sequence alone.

### Example 92

The following C. pneumoniae protein (PID 4377408) was expressed <SEQ ID 183; cp7408>:

| | |
|---|---|
| 1 | MLKIQKKRMC VSVVITVGAI VGFFNSADAA PKKKKIPIQI LYSFTKVSSY |
| 51 | LKNEDASTIF CVDVDRGLLQ HRYLGSPGWQ ETRRRQLFKS LENQSYGNER |
| 101 | LGEETLAIDI FRNKECLESE IPEQMEAILA NSSALVLGIS SFGITGIPAT |
| 151 | LHSLLRQNLS FQKRSIASES FLLKIDSAPS DASVFYKGVL FRGETAIVDA |
| 201 | LSQLFAQLDL SPKKIIFLGE DPEVVQAVGS ACIGWGMNFL GLVYYPAQES |
| 251 | LFSYVHPYST ATELQEAQGL QVISDEVAQL TLNALPKMN* |

The cp7408 nucleotide sequence <SEQ ID 184> is:

| | |
|---|---|
| 1 | ATGTTGAAAA TCCAGAAAAA AAGAATGTGT GTCAGCGTAG TCATCACGGT |
| 51 | AGGCGCCATA GTGGGGTTTT TCAATTCTGC AGACGCAGCA CCAAAGAAAA |
| 101 | AGAAGATCCC TATACAGATT CTCTACTCCT TTACTAAAGT CTCTTCCTAT |
| 151 | TTAAAAAACG AAGACGCAAG TACTATATTT TGCGTCGATG TGGATCGTGG |
| 201 | ACTTCTCCAG CATCGGTATT TAGGTAGTCC AGGATGGCAG GAAACCAGAC |
| 251 | GTCGGCAGTT ATTTAAATCC TTAGAAAATC AATCATACGG CAACGAACGT |
| 301 | TTAGGAGAAG AAACTCTTGC TATTGATATT TTCAGGAACA AAGAGTGCTT |
| 351 | GGAGAGCGAG ATCCCAGAGC AGATGGAAGC TATCCTTGCA AATTCCTCGG |
| 401 | CCTTGGTCTT AGGCATCTCT TCTTTTGGGA TCACAGGAAT TCCTGCGACT |
| 451 | TTGCATAGTT TGCTTCGACA GAATCTATCT TTCCAAAAAC GCTCTATAGC |
| 501 | ATCGGAGAGC TTCCTTTTAA AGATCGATAG TGCCCCCTCA GATGCCTCTG |
| 551 | TTTTTTATAA AGGCGTGCTT TTCCGCGGAG AGACTGCGAT CGTGGATGCG |
| 601 | TTAAGCCAAT TATTTGCCCA GCTCGATCTT TCTCCTAAAA AAATTATCTT |
| 651 | TCTAGGAGAA GACCCTGAGG TCGTTCAAGC TGTTGGGTCT GCTTGTATAG |
| 701 | GTTGGGGCAT GAACTTTTVA GGCCTGGTAT ACTATCCTGC TCAAGAAAGC |
| 751 | CTTTTTTCTT ATGTTCATCC TTACTCTACA GCAACGGAGC TCCAAGAAGC |
| 801 | ACAGGGTTTA CAAGTAATTT CAGATGAAGT CGCACAGCTT ACTTTAAACG |
| 851 | CTCTTCCGAA AATGAATTAA |

The PSORT algorithm predicts inner membrane (0.123).

The protein was expressed in E. coli and purified as a his-tag product (FIG. 92A). The recombinant protein was used to immunise mice, whose sera were used in a Western blot (FIG. 92B) and for FACS analysis.

These experiments show that cp7408 is a surface-exposed and immunoaccessible protein, and that it is a useful immunogen. These properties are not evident from the sequence alone.

### Example 93

The following C. pneumoniae protein (PID 4376444) was expressed <SEQ ID 185; cp6424>:

| | |
|---|---|
| 1 | MMHNIVVLSE EPGRSAFLGR TAFFPNKYPI AQGGVGIPST IGNLFTIWYC |
| 51 | FYFYRAATPQ SDHPDGCGFI LLERLKELGA GFFYCDLRES NTTGFTDFFE |
| 101 | GSNKGVLKNH LFIRDE* |

The cp6424 nucleotide sequence <SEQ ID 186> is:

| | |
|---|---|
| 1 | ATGATGCACA ATATTGTTGT TCTTAGTGAG GAACCTGGAC GAAGCGCTTT |
| 51 | TCTTGGTAGG ACGGCATTTT TCCCTAATAA GTATCCAATA GCTCAGGGTG |
| 101 | GTGTTGGAAT ACCATCTACA ATAGGCAATC TCTTTACTAT ATGGTACTGT |
| 151 | TTCTATTTTT ATAGAGCTGC AACTCCACAA TCTGATCATC CTGACGGATG |
| 201 | TGGCTTTATT CTACTAGAAA GGCTTAAGGA GCTCGGTGCA GGGTTCTTTT |
| 251 | ATTGTGATCT TCGTGAGTCC AATACCACTG GCTTTACTCT TTTTTTTGAA |
| 301 | GGCTCCAATA AAGGTGTGTT AAAGAATCAC TTGTTTATTA GAGATGAGTA |
| 351 | A |

The PSORT algorithm predicts cytoplasm (0.2502).

The protein was expressed in E. coli and purified as a GST-fusion product (FIG. 93A) and also in his-tagged form. The recombinant proteins were used to immunise mice, whose sera were used in Western blots (FIG. 93B) and for FACS analyses (FIG. 93C; GST-fusion).

These experiments show that cp6424 is a surface-exposed and immunoaccessible protein, and that it is a useful immunogen. These properties are not evident from the sequence alone.

### Example 94

The following C. pneumoniae protein (PID 4376449) was expressed <SEQ ID 187; cp6449>:

| | |
|---|---|
| 1 | VASETYPSQI LHAQREVRDA YFNQADCHPA RANQILEAKK ICLLDVYHTN |
| 51 | HYSVFTFCVD NYPNLRFTFV SSKNNEMNGL SNPLDNVLVE AMVRRTHARN |
| 101 | LLAACKIRNI EVPRVVGLDL RSGILISRLE LKQPQFQSLT EDFVNHSTNQ |
| 151 | EEARVHQKHV LLISLILLCK QAVLESFQEK KRSS* |

The cp6449 nucleotide sequence <SEQ ID 188> is:

| | |
|---|---|
| 1 | GTGGCGTCTG AAACGTATCC TTCTCAGATA TTGCACGCTC AGAGGGAAGT |
| 51 | ACGTGATGCC TATTTTAATC AAGCGGATTG CCATCCTGCT CGGGCTAATC |
| 101 | AGATTCTCGA GGCTAAGAAA ATCTGTTTAT TAGATGTTTA TCATACTAAT |
| 151 | CATTATTCCG TATTTACTTT TTGTGTAGAT AATTATCCGA ATCTCCGCTT |
| 201 TACATTTTGTA TCTTCAAAAA ACAATGAGAT GAATGGCTTA TCTAATCCTC | |
| 251 | TAGATAATGT TCTTGTAGAG GCTATGGTAC GTAGAACACA TGCAAGAAAC |
| 301 | CTACTTGCAG CGTGTAAAAT TCGAAATATT GAGGTTCCAA GGGTTGTTGG |
| 351 | GCTTGACCTA AGATCTGGGA TACTCATTTC GAAACTAGAA TTGAAGCAAC |
| 401 | CTCAGTTCCA AAGTTTAACA GAAGACTTCG TAAATCATTC CACAAATCAG |
| 451 | GAAGAAGCTC GCGTCCATCA AAAGCATGTG TTGCTAATTT CTTTAATTTT |
| 501 | ACTTTGCAAC CAGGCCGTTC TGGAATCATT CCAGGAAAAA AAGCGATCCT |
| 551 | CTTAA |

The PSORT algorithm predicts inner membrane (0.2084).

The protein was expressed in E. coli and purified as a GST-fusion product (FIG. 94A) and also in his-tagged form. The recombinant proteins were used to immunise mice, whose sera were used in Western blots (FIG. 94B) and for FACS analyses (FIG. 94C; GST-fusion).

These experiments show that cp6449 is a surface-exposed and immunoaccessible protein, and that it is a useful immunogen. These properties are not evident from the sequence alone.

### Example 95

The following C. pneumoniae protein (PID 4376495) was expressed <SEQ ID 189; cp6495>:

MRELNAFELTQPEEYRNRWVLMPCLKCRFCRTQHAKVWSYRCVHEASLYE
KNCFLTLTYDDKHLPQYGSLVKLHLQLFLKRLRKMISPHKIRYFECGAYG
TKLQRPHYHLLLS

The cp6495 nucleotide sequence <SEQ ID 190> is:

TTGCGAGAATTAAATGCTTTTGAATTAACTCAACCTGAAGAGTATCGAAACCGTTGGGTTTTGATGCCTTGTCTTAAGTGT
CGTTTTTGTAGAACGCAACATGCAAAAGTCTGGTCTTATCGTTGTGTCCATGAAGCTTCTTTGTATGAGAAAAATTGTTTT
CTTACTTTGACTTATGATGATAAGCATTTACCTCAGTATGGTTCGTTGGTAAAGCTGCATTTACAGCTGTTTCTTAAGAGA
TTAAGAAAGATGATTTCTCCTCATAAAATTCGTTATTTTGAATGTGGTGCGTATGGAACCAAATTACAAAGACCTCATTAT
CATCTACTTTTATCATGA

The PSORT algorithm predicts cytoplasmic (0.280).

The protein was expressed in E. coli and purified as a GST-fusion product (FIG. 95A). The recombinant protein was used to immunise mice, whose sera were used in a Western blot (FIG. 95B) and for FACS analysis (FIG. 95C).

These experiments show that cp6495 is a surface-expose and immunoaccessible protein, and that it is a useful immunogen. These properties are not evident from the sequence alone.

### Example 96

The following C. pneumoniae protein (PID 4376506) was expressed <SEQ ID 191; cp6506>:

| | |
|---|---|
| 1 | MRRFLFLILS SLPLVAFSAD NFTILEEKQS PLSRVSIIFA LPGVTPVSFD |
| 51 | GNCPIPWFSH SKKTLEGQRI YYSGDSFGKY FVVSALWPNK VSSAVVACNM |
| 101 | ILKHRVDLIL IIGSCYSRSQ DSRPGSVLVS KGYINYDADV RPFFERFEIP |
| 151 | DIKKSVFATS EVHREAILRG GEEFISTHKQ EIEELLKTHG YLKSTTKTEH |
| 201 | TLMEGLVATG ESFAMSRNYF LSLQKLYPEI HGFDSVSGAV SQVCYEYSIP |
| 251 | CLGVNILLPH PLESRSNEDW KHLQSEASKI YMDTLLKSVL KELCSSH* |

The cp6506 nucleotide sequence <SEQ ID 192> is:

| | |
|---|---|
| 1 | ATGCGTCGTT TTCTGTTTCT TATTCTTAGC TCTCTTCCTT TGGTCGCATT |
| 51 | CTCTGCTGAT AATTTPCACTA TTCTAGAAGA AAATCAGAGT CCTTTAAGTC |
| 101 | GTGTAAGTAT TATTTTTGCT TTACCTGGGG TTACTCCCGT TTCTTTTGAT |
| 151 | GGTAATTGTC CTATTCCTTG GTTTTCTCAT AGTAAAAAGA CTCTAGAGGG |
| 201 | ACAGAGAATT TATTACTCTG GCGACTCCTT TGGGAAATAC TTTGTAGTTT |
| 251 | CTGCTCTTTG GCCTAATTAA GTTTCTTCAG CTGTTGTGGC TTGTAATATG |
| 301 | ATTCTTAAAC ATCGAGTGGA TCTTATTCTA ATTATAGGCT CGTGTTACTC |
| 351 | TAGGTCTCAA GATAGCCGTT TTGGCAGCGT CTTAGTTTCT AAAGGCTACA |
| 401 | TTAATTATGA TGCAGATGTG AGGCCTTTCT TTGAAAGATT TGAGATTCCA |
| 451 | GACATTAAAA AGAGTGTTTT TGCAACCAGT GAGGTTCATC GGGAGGCAAT |
| 501 | TCTTCGTGGA GGCGAAGAGT TTATTTCTAC CCATAAACAA GAAATCGAAG |
| 551 | AGCTTTTGAA GACTCATGGG TATTTGAAAT CAACAACCAA AACGGAGCAC |
| 601 | ACCTTAATGG AAGGTTTGGT TGCTACAGGC GAGTCTTTCG CGATGTCGCG |
| 651 | AAACTATTTT CTTTCCTTAC AAAAATTGTA TCCAGAGATT CATGGTTTTG |
| 701 | ATAGTGTCAG CGGCGCTGTT TCTCAGGTAT GCTATGAATA TAGCATTCCT |
| 751 | TGTTTAGGTG TGAATATCCT TCTCCCTCAT CCTTTAGAAT CACGGAGTAA |
| 801 | CGAGGATTGG AAGCATCTTC AAAGTGAGGC AAGTAAAATT TATATGGATA |
| 851 | CCTTGCTCAA GAGTGTATTA AAAGAACTCT GTTCTTCTCA TTAA |

The PSORT algorithm predicts periplasmic spare (0.571).

The protein was expressed in E. coli and purified as his-tag (FIG. 96A) and GST-fusion (FIG. 96B) products. The GST-fusion protein was used to immunise mice, whose sera were used in a Western blot (FIG. 96C) and for FACS analysis (FIG. 96D)).

These experiments show that cp6506 is a surface-exposed and immunoaccessible protein, and that it is a useful immunogen. These properties are not evident from the sequence alone.

### Example 97

The following C. pneumoniae protein (PID 4376882) was expressed <SEQ ID 193; cp6882>:

| | |
|---|---|
| 1 | MSLLNLPSSQ DSASEDSTSQ SQIFDPIRNR ELVSTPEEKV RQRLLSFLMH |
| 51 | KLNYPKKLII IEKELKTLFP LLMRKGTLIP KRRPDILIIT PPTYTDAQGN |
| 101 | THNLGDPKPL LLIECKALAV NQNALKQLLS YNYSIGATCI AMAGKHSQVS |
| 151 | ALFNPKTQTL DFYPGLPEYS QLLNYFISLN L* |

The cp6882 nucleotide sequence <SEQ ID 194> is:

| | |
|---|---|
| 1 | ATGTCCTTAT TGAACCTTCC CTCAAGCCAG GATTCTGCAT CTGAGGACTC |
| 51 | CACATCGCAA TCTCAAATCT TCGATCCCAT TAGAAATCGG GAGTTAGTTT |
| 101 | CTACTCCCGA AGAAAAAGTC CGCCAAAGGT TGCTCTCCTT CCTAATGCAT |
| 151 | AAGCTGAACT ACCCTATGAA ACTCATCATC ATAGAAAAAG AACTCAAAAC |
| 201 | TCTTTTTCCT CTGCTTATGC GTAAAGGAAC CCTAATCCCA AAACGCCGCC |
| 251 | CAGATATTCT CATCATCACT CCCCCCACAT ACACAGACGC ACAGGGAAAC |
| 301 | ACTCACAACC TAGGCGACCC AAAACCCCTG CTACTTATCG AATGTAAGGC |
| 351 | CTTAGCCGTA AACCAAAATG CACTCAAACA ACTCCTTAGC TATAACTACT |
| 401 | CTATCGGAGC CACCTGCATT GCTATGGCAG GGAAACACTC TCAATTGTCA |
| 451 | GCTCTCTTCA ATCCAAAAAC ACAAACTCTT GATTTTTATC CTGGCCTCCC |
| 501 | AGAGTATTCC CAACTCCTAA ACTACTTTAT TTCTTTAAAC TTATAG |

The PSORT algorithm predicts cytoplasm (0.362).

The protein was expressed in E. coli and purified as a GST-fusion product (FIG. 97A). The protein was used to immunise mice, whose sera were used in a Western blot (FIG. 97B) and for FACS analysis (FIG. 97C).

These experiments show that cp6882 is a surface-exposed and immunoaccessible protein, and that it is a useful immunogen. These properties are not evident from the sequence alone.

### Example 98

The following C. pneumoniae protein (PID 4376979) was expressed <SEQ ID 195; cp6979>:

| | |
|---|---|
| 1 | MSVNPSGNSK NDLWITGAHD QHPDVKESGV TSANLGSHRV TASGGRQGLL |
| 51 | ARIKEAVTGF FSRMSFFRSG APRGSQQPSA PSADTVRSPL PGGDARATEG |
| 101 | AGRNLIKKGY QPGMKVTIPQ VPGGGAQRSS GSTTLKPTRP APPPPKTGGT |
| 151 | NAKRPATHGK GPAPQPPKTG GTNAKRAATH GKGPAPQPPK GILKQPGQSG |
| 201 | TSGKKRVSWS DED* |

The cp6979 nucleotide sequence <SEQ ID 196> is:

| | |
|---|---|
| 1 | ATGTCTGTTA ATCCATCAGG AAATTCCAAG AACGATCTCT GGATTACGGG |
| 51 | AGCTCATGAT CAGCATCCCG ATTTTAAAGA ATCCGGGGTT ACAAGTGCTA |
| 101 | ACCTAGGAAG TCATAGAGTG ACTGCCTCAG GAGGACGCCA AGGGTTATTA |
| 151 | GCACGAATCA AAGAAGCAGT AACCGGGTTT TTTAGTCGGA TGAGCTTCTT |
| 201 | CAGATCGGGA GCTCCAAGAG GTAGCCAACA ACCCTCTGCT CCATCTGCAG |
| 251 | ATACTGTACG TAGCCCGTTG CCGGGAGGGG ATGCTCGCGC TACCGAGGGA |
| 301 | GCTGGTAGGA ACTTAATTAA AAAAGGGTAC CAACCAGGGA TGAAAGTCAC |
| 351 | TATCCCACAG GTTCCTGGAG GAGGGGCCCA ACGTTCATCA GGTAGCACGA |
| 401 | CACTAAAGCC TACGCGTCCG GCACCCCCAC CTCCTAAAAC GGGTGGAACT |
| 451 | AATGCAAAAC GTCCGGCAAC GCACGGGAAG GGTCCAGCAC CCCAGCCTCC |
| 501 | TAAAACAGGT GGGACCAATG CTAAGCGCGC AGCAACGCAT GGGTAAGGTC |
| 551 | CAGCACCTCA ACCTCCTAAG GGCATTTTGA AACAGCCTGG GCAGTCTGGG |
| 601 | ACTTCAGGAA AGAAGCGTGT CAGCTGGTCT GACGAAGATT AA |

The PSORT algorithm predicts cytoplasm (0.360).

The protein was expressed in E. coli and purified as a GST-fusion product (FIG. 98A). The GST-fusion protein was used to immunise mice, whose sera were used in a Western blot (FIG. 98B) and for FACS analysis (FIG. 98C).

These experiments show that cp6979 is a surface-exposed and immunoaccessible protein, and that it is a useful immunogen. These properties are not evident from the sequence alone.

### Example 99

The following C. pneumoniae protein (PID 4377028) was expressed <SEQ ID 197; cp7028>:

| | |
|---|---|
| 1 | MLLGFLCDCP CASWQCAAVA NCYDSVFMSR PEHKPNIPYI TKATRRGLRM |
| 51 | KTLAYLASLK DARQLAYDFL KDPGSLARLA KALIAPKEAL QEGNLFFYGC |
| 101 | SNIEDILEEM RRPHRILLLG FSYCQKPKAC PEGRPNDACR YDPSHPTCAS |
| 151 | CSIGTMMRLN ARRYTTVIIP TFIDIAKHLH TLKKRYPGYQ ILFAVTACEL |
| 201 | SLKMFGDYAS VMNLKGVGIR LTGRICNTFK AFKLAERGVK PGVTILEEDG |
| 251 | FEVLARILTE YSSAPFPRDF CEIH* |

The cp7028 nucleotide sequence <SEQ ID 198> is:

| | |
|---|---|
| 1 | ATGCTTCTAG GGTTTTTGTG TGACTGCCCC TGTGCTTCGT GGCAGTGTGC |
| 51 | GGCCGTTGCT AATTGTTATG ATTCCGTATT TATGTCTAGA CCAGAGCACA |
| 101 | AACCTAATAT TCCTTATATT ACTAAAGCTA CAAGACGGGG TCTGCGTATG |
| 151 | AAGACGCTTG CTTATCTGGC CTCTTTAAAA GATGCTAGAC AGCTTGCCTA |
| 201 | TGATTTTCTG AAAGATCCTG GTTCTTTAGC TCGGTTAGCT AAGGCTTTGA |
| 251 | TAGCTCCTAA GGAGGCCTTA CAGGAGGGCA ACCTATTTTT TTATGGCTGT |
| 301 | AGTAATATTG AGGATATTTT AGAGGAGATG CGTCGTCCTC ATAGAATCCT |
| 351 | TTTGTTAGGA TTTTCTTATT GTCAAAAGCC TAAGGCATGT CCTGAATGGC |
| 401 | GTTTCTATGA TGCTTGTCGG TATGATCCTT CACATCCTAC ATGTGCCTCA |
| 451 | TGTTCTATAG GGACCATGAT GCGGCTGAAT GCTCGTAGAT ACACTACTGT |
| 501 | GATCATCCCT ACATTTATAG ATATCGCAAA ACATTTACAC ACTTTAAAAA |
| 551 | AGCGCTACCC TGGATATCAA ATTCTCTTTG CAGTTACTGC TTGTGAACTT |
| 601 | TCCTTAAAAA TGTTTGGATA TTATGCCTCC GTAATGAACT TAAAGGGTGT |
| 651 | GGGCATCAGA CTCACAGGAC GTATTTGCAA TACATTTAAG GCATTTAAAT |
| 701 | TAGCTGAGCG AGGAGTCAAA CCAGGAGTCA CTATCCTAGA AGAAGATGGC |
| 751 | TTTGAGGTAT TAGCAAGGAT TCTTACAGAA TACAGTAGCG CTCCTTTCCC |
| 801 | TAGAGACTTT TGTGAGATCC ATTAG |

The PSORT algorithm predicts cytoplasm (0.1453).

The protein was expressed in E. coli and purified as a GST-fusion product (FIG. 99A). The recombinant protein was used to immunise mice, whose sera were used in a Western blot (FIG. 99B) and for FACS analysis (FIG. 99C).

These experiments show that cp7028 is a surface-exposed and immunoaccessible protein, and that it is a useful immunogen. These properties are not evident from the sequence alone.

### Example 100

The following C. pneumoniae protein (PID 4377355) was expressed <SEQ ID 199; cp7355>:

| | |
|---|---|
| 1 | MKKVVTLSII FFATYCASEL SAVTVVAVPL SEAPGKIQVR PVVGLQFQEE |
| 51 | QGSVPYSFYY PYDYGYYYPE TYGYTKNTGQ ESRECYTRFE DGTIFYECD* |

The cp7355 nucleotide sequence <SEQ ID 200> is:

| | |
|---|---|
| 1 | ATGAAGAAAG TCGTAACACT ATCCATTATA TTTTTCGCAA CGTATTGTGC |
| 51 | ATCAGAGCTT AGTGCTGTAA CTGTAGTGGC TGTGCCTTTA TCAGAGGCTC |
| 101 | CAGGGAAGAT TCAAGTTCGT CCCGTCGTTG GTCTGCAATT TCAAGAAGAA |
| 151 | CAGGGTTCTG TGCCCTATAG TTTTTATTAT CCTTATGACT ATGGGTATTA |
| 201 | CTATCCAGAG ACTTATGGCT ATACTAAAAA TACAGGTCAA GAAAGTCGCG |
| 251 | AATGTTATAC CCGATTTGAA GATGGCACAA TTTTTTATGA ATGCGATTAG |

The PSORT algorithm predicts inner membrane (0.143).

The protein was expressed in E. coli and purified as a GST-fusion (FIG. 100A) and a his-tag product. The proteins were used to immunise mice, whose sera were used in a Western blot (FIG. 100B) and for FACS analysis (FIG. 100C).

These experiments show that cp7355 is a surface-exposed and immunoaccessible protein, and that it is a useful immunogen. These properties are not evident from the sequence alone.

### Example 101

The following C. pneumoniae protein (PID 4377380) was expressed <SEQ ID 201; cp7380>:

| | |
|---|---|
| 1 | VHYCERTLDP KYILKIALKL RQSLSLFFQN SQSLQHAYST PYSYYRIILQ |
| 51 | KENKEKQALA RHKCISILEF FKNLLFVHLL SLSKNQREGC STDMAVVSTP |
| 101 | FFNRNLWYRL LSSRFSLWKS YCPRFFLDYL EAFGLLSDFL DHQAVIKFFE |
| 151 | LETHFSYYPV SGFVAPHQYL SLLQDRYFPI ASVMRTLDKD NFSLTPDLIH |
| 201 | DLLGHVPWLL HPSFSEFFIN MGRLFTKVIE KVQALPSKKQ RIQTLQSNLI |
| 251 | AIVRCFWFTV ESGLIENHEG RKAYGAVLIS SPQELGHAFI DNVRVLPLEL |
| 301 | DQIIRLPFNT STPQETLFSI RHFDELVELT SKLEWMLDQG LLESIPLYNQ |
| 351 | EKYLSGFEVL CQ* |

The cp7380 nucleotide sequence <SEQ ID 202> is:

| | |
|---|---|
| 1 | GTGCACTACT GCGAGAGAAC CCTGGACCCA AAGTATATTC TGAAGATTGC |
| 51 | TCTAAAGCTG AGACAATCAC TTTCCCTGTT CTTCCAGAAC AGCCAATCAC |
| 101 | TCCAACGTGC ATACTCGACC CCATATTCCT ACTACCGAAT CATTCTACAA |
| 151 | AAGGAAAATA AAGAGAAGCA AGCTTTAGCT CGACACAAAT GCATTTCTAT |
| 201 | TTTAGAATTT TTCAAAAACT TACTCTTTGT TCATCTTCTG TCATTATCAA |
| 251 | AGAATCAAAG GGAAGGTTGC TCCACTGATA TGGCTGTTGT AAGCACTCCC |
| 301 | TTTTTTAATC GGAATTTATG GTATCGACTC CTTTCCTCAC GGTTTTCTCT |
| 351 | ATGGAAAAGC TATTGTCCAA GATTTTTTCT TGATTACTTA GAAGCTTTCG |
| 401 | GTCTCCTTTC TGATTTCTTA GACCATCAAG CAGTCATTAA ATTCTTCGAA |
| 451 | TTAGAAACAC ATTTTTCCTA TTATCCCGTT TCAGGATTTG TAGCTCCCCA |
| 501 | TCAATACTTG TCTCTGTTGC AGGACCGTTA CTTTCCCATT GCCTCTGTAA |
| 551 | TGCGAACTCT CGATAAAGAT AATTTCTCCT TAACTCCTGA TCTCATCCAT |
| 601 | GACCTTTTAG GGCACGTGCC TTGGCTTCTA CATCCCTCAT TTTCTGAATT |
| 651 | TTTCATAAAC ATGGGAAGAC TCTTCACTAA AGTCATAGAA AAAGTACAAG |
| 701 | CTCTTCCTAG TAAAAAACAA CGCATACAAA CCCTACAAAG CAATCTGATC |
| 751 | GCTATTGTAC GCTGCTTTTG GTTTACTGTT GAAAGCGGAC TTATTGAAAA |
| 801 | CCATGAAGGA AGAAAAGCAT ATGGAGCCGT TCTTATCAGT TCTCCTCAGG |
| 851 | AACTTGGACA CGCTTTCATT GATAACGTAC GTGTTCTCCC TTTAGAATTG |
| 901 | GATCAGATTA TTCGTCTTCC CTTCAATACA TCAACTCCAC AAGAGACTTT |
| 951 | ATTTTCAATA AGACATTTTG ATGAACTGGT AGAACTCACT TCAAAATTAG |
| 1001 | AATGGATGCT CGACCAAGGT CTGTTAGAAT CAATTCCCCT TTACAATCAA |
| 1051 | GAGAAATATC TTTCTGGTTT TGAGGTACTT TGCCAATGA |

The PSORT algorithm predicts inner membrane (0.1362).

The protein was expressed in E. coli and purified as a GST-fusion product (FIG. 101A). The recombinant protein was used to immunise mice, whose sera were used in a Western blot (FIG. 101B) and for FACS analysis (FIG. 101C).

These experiments show that cp7380 is a surface-exposed and immunoaccessible protein, and that it is a useful immunogen. These properties are not evident from the sequence alone.

### Example 102

The following C. pneumoniae protein (PID 4376904) was expressed <SEQ ID 203; cp6904>:

| | |
|---|---|
| 1 | MMNYEDAKLR GQAVAILYQI GAIKFGKHIL ASGEETPLYV DMRLVISSPE |
| 51 | VLQTVATLIW RLRPSFNSSL LCGVPYTALT LATSISLKYN IPMVLRRKEL |
| 101 | QNVDPSDAIK VEGLFTPGQT CLVINDMVSS GKSIIETAVA LEENGLVVRE |
| 151 | ALVFLDRRKE ACQPLGPQGI KVSSVFTVPT LIKALIAYGK LSSGDLTLAN |
| 201 | KISEILEIES * |

The cp6904 nucleotide sequence <SEQ ID 204> is:

| | |
|---|---|
| 1 | ATGATGAACT ACGAAGATGC AAAATTACGC GGTCAAGCTG TAGCAATTCT |
| 51 | ATACCAAATC GGAGCTATAA AGTTCGGAAA ACATATTCTC GCTAGCGGAG |
| 101 | AAGAAACTCC TCTGTATGTA GATATGCGTC TTGTGATCTC CTCTCCAGAA |
| 151 | GTTCTCCAGA CAGTGGCAAC TCTTATTTGG CGCCTCCGCC CCTCATTCAA |
| 201 | TAGTAGCTTA CTCTGCGGAG TCCCTTATAC TCCTCTAACC CTAGCAACCT |
| 251 | CGATCTCTTT AAAATATAAC ATCCCTATGG TATTGCGAAG GAAGGAATTA |
| 301 | CAGAATGTAG ACCCCTCGGA CGCTATTAAA GTAGAAGGGT TATTTACTCC |
| 351 | AGGACAAACT TGTTTAGTCA TCAATGATAT GGTTTCCTCA GGAAAATCTA |
| 401 | TAATAGAGAC AGCAGTCGCA CTGGAAGAAA ATGGTCTGGT AGTTCGTGAA |
| 451 | GCATTGGTAT TCTTAGATCG TAGAAAAGAA GCGTGTCAAC CACTTGGTCC |
| 501 | ACAGGGAATA AAAGTCAGTT CGGTATTTAC TGTACCCACT CTGATAAAAG |
| 551 | CTTTGATCGC TTATGGGAAG CTAAGCAGTG GTGATCTAAC CCTGGCAAAC |
| 601 | AAAATTTCCG AAATTCTAGA AATTGAATCT TAA |

The PSORT algorithm predicts cytoplasm (0.0358).

The protein was expressed in E. coli and purified as a his-tag product (FIG. 102A). The recombinant protein was used to immunise mice, whose sera were used in a Western blot (FIG. 102B) and for FACS analysis.

The cp6904 protein was also identified in the 2D-PAGE experiment.

These experiments show that cp6904 is a surface-exposed and immunoaccessible protein, and that it is a useful immunogen. These properties are not evident from the sequence alone.

### Example 103

The following C. pneumoniae protein (PID 4376964) was expressed <SEQ ID 205; cp6964>:

| | |
|---|---|
| 1 | MKKLIALIGI FLVPTKGNTN KEHDAHATVL KAARAKYNLF PVQDVFPVHE |
| 51 | VIEPISPDCL VHYEGWV* |

The cp6964 nucleotide sequence <SEQ ID 206> is:

| | |
|---|---|
| 1 | ATGAAAAAAT TGATTGCTTT GATAGGGATA TTTCTTGTTC CAATAAAAGG |
| 51 | AAATACCAAT AAGGAACACG ACGCTCACGC GACTGTTTTA AAAGCGGCCA |
| 101 | GAGCAAAGTA TAATTTGTTC TTTGTTCAGG ATGTTTTCCC TGTACACGAA |
| 151 | GTTATCQAGC CTATTTCTCC CGAT~PGCCTG GTACATTATG AAGGGTGGGT |
| 201 | TTGA |

The PSORT algorithm predicts inner membrane (0.091).

The protein was expressed in E. coli and purified as a GST-fusion product (FIG. 103A) and also in his-tagged form. The recombinant proteins were used to immunise mice, whose sera were used in a Western blot (FIG. 103B) and for FACS analysis (FIG. 103C).

These experiments show that cp6964 is a surface-exposed and immunoaccessible protein, and that it is a useful immunogen. These properties are not evident from the sequence alone.

### Example 104

The following C. pneumoniae protein (PID 4377387) was expressed <SEQ ID 207; cp7387>:

| | |
|---|---|
| 1 | LNFAKIDHNH LYLTCLGDLG VACPILSTDC LPNYSEKASH EVLVYSKFRC |
| 51 | ISGEPSRLAT SGNDTYYSIV SLPIGLRYEV TSPSGRHDFN IDMHVAPKIG |
| 101 | AVLSHGTREA KEIPGSSKDY AFFSLTARES LMISEKLAMT FQVSEVIQNC |
| 151 | YSQCTKVWKT NLKEQYRHLS HNTGFELSVK SAF* |

The cp7387 nucleotide sequence <SEQ ID 208> is:

| | |
|---|---|
| 1 | TTGAATTTTG CAAAGATTGA TCACAATCAT CTCTACCTTA CATGTTTGGG |
| 51 | AGATCTTGGT GTAGCTTGTC CTATACTTTC TACAGATTGT CTACCTAATT |
| 101 | ATAGCGAGAA AGCATCTCAT GAGGTTCTTG TTTATAGTAA ATTTAGATGC |
| 151 | ATTTCTGGAG AGCCATCTCG ACTTGCAACT TCAGGAAATG AGACATATTA |
| 201 | TTCTATAGTA AGTTTACCTA TAGGACTCCG TTACGAAGTG ACTTCACCAT |
| 251 | CAGGACGTCA TGATTTCAAT ATTGATATGC ATGTAGCTCC AAAGATAGGT |
| 301 | GCAGTACTCT CTCATGGAAC ACGAGAGGCT AAAGAGATCC CAGGATCTTC |
| 351 | AAAAGACTAT GCATTTTTTA GCTTGACPGC TAGAGAAAGT TTAATGATTT |
| 401 | CTGAAAAGCT TGCGATGACT TTCCAAGTTA GCGAAGTTAT TCAGAATTGT |
| 451 | TATTCACAAT GTACTAAACT AACGAAAACT AATTTAAAAG AACAGTATAG |
| 501 | GCACTTATCC CACAATACAG GGTTTGAGTT AAGCGTCAAG TCTGCATTCT |
| 551 | AA |

The PSORT algorithm predicts inner membrane (0.043).

The protein was expressed in E. coli and purified as a his-tagged-fusion product (FIG. 104A) and also as a GST-fusion (FIG. 104B). The recombinant proteins were used to immunise mice, whose sera were used in a Western blot and for FACS analysis (FIG. 104C; his-tagged).

These experiments show that cp7387 is a surface-exposed and immunoaccessible protein, and that it is a useful immunogen. These properties are not evident from the sequence alone.

### Example 105

The following C. pneumoniae protein (PID 4376281) was expressed <SEQ ID 209; cp6281>:

| | |
|---|---|
| 1 | MFLQFFHPIV FSDQSLSFLP YLGKSSGIIE KCSNIVEHYL HLGGDTSVII |
| 51 | TGVSGATFLS VDHALPISKS EKIIKILSYI LILPLILALE IKIVLRIILF |
| 101 | FKYRGLILDV KKEDLKKTLT PDQENLSLPL PSPTTLKKIH ALHILVRSGK |
| 151 | TYNELIQEGP SFTKITDLGQ APSPKQDIGF SYNSLLPNFY FHSLVSVPNI |
| 201 | SGEERALNYH KBQQEEMAVK LKTMQACSFV FRSLHLPSMQ TKDKKAGFGL |
| 251 | LTFFPWKIYP L* |

The cp6281 nucleotide sequence <SEQ ID 210> is:

| | |
|---|---|
| 1 | ATGTTTCTTC AGTTTTTTCA TCCTATAGTC TTCTCGGATC AGTCCTTATC |
| 51 | TTTTCTTCCT TACCTAGGAA AAAGCTCTGG CATTATTGAA AAATGTTCCA |
| 101 | ATATCGTTGA ACACTATTTA CATTTGGGAG GAGACACTTC TGTTATCATC |
| 151 | ACAGGAGTTT CTGGAGCTAC CTTTCTATCT GTTGATCATG CCCTCCCAAT |
| 201 | CTCGAAATCT GAAAAAATAA TAAAAATTCT CTCCTATATT TTAATTCTTC |
| 251 | CTCTGATTCT AGCTCTCTTT ATTAAAATCG TTTTACGCAT TATCTTATTC |
| 301 | TTCAAGTATC GTGGTCTAAT CCThGATGTT AAGAAGGAGG ATTTGAAAAA |
| 351 | AACACTTACA CCTGACCAAG AAAACCTCAG TCTTCCTTTA CCATCTCCTA |
| 401 | CAACATTAAA GAAAATTCAT GCGCTACACA TTTTAGTGCG TTCTGGAAAA |
| 451 | ACCTATAACG AGCTTATACA AGAAGGGTTT TCTTTCACTA AAATCACAGA |
| 501 | TCTTGGTCAA GCTCCTTCAC CAAAGCAAGA TATTGGCTTC TCTTATAATT |
| 551 | CCCTTCTCCC TAACTTCTAT TTTCATTCCT TGGTATCTGT TCCAAATATT |
| 601 | TCAGGCGAGG AACGGGCTCT TAATTATCAT AAAGAACAAC AAGAGGAAAT |
| 651 | GGCTGTTAAA TTAAAAACAA TGCAAGCGTG TTCTTTTGTC TTCCGATCCC |
| 701 | TGCATTTACC TTCAATGCAA ACGAAGGACA AAAAGGCTGG ATTTGGACTA |
| 751 | CTGACGTTTT TCCCTTGGAA AATCTACCCC CTATAA |

The PSORT algorithm predicts inner membrane (0.5373).

The protein was expressed in E. coli and purified as a GST-fusion product (FIG. 105A). The recombinant protein was used to immunise mice, whose sera were used in a Western blot (FIG. 105B) and for FACS analysis.

These experiments show that cp6281 is a surface-exposed and immunoaccessible protein, and that it is a useful immunogen. These properties are not evident from the sequence alone.

### Example 106 and Example 107

The following C. pneumoniae protein (PID 4376306) was expressed <SEQ ID 211; cp6306>:

| | |
|---|---|
| 1 | MGNHBTYIHP GVLPSSHAQD VSRSTVYPSR SFIMRRHLMG WNFNRVPSKS |
| 51 | SEQLMDGHRI PLIFFGKHHP TISILNVNRF SWLSIFYNGE RGF* |

The cp6306 nucleotide sequence <SEQ ID 212> is:

| | |
|---|---|
| 1 | ATGGGAAACC ATGAGACCTA TATACATCCA GGAGTGCTCC CGAGTAGTCA |
| 51 | TGCTCAGGAT GTTAGCAGAT CTACAGTTTA CCCCAGTCGA AGTTTTATCA |
| 101 | TGAGACGTAT GCTCATGGGC TGGAATTTCA ATCGTGTTCC CTCGAAGAGC |
| 151 | TCCGAGCAGT TAATGGATGG TCATCGCATA CCTCTTATAT TTTTTGGGAA |
| 201 | GCATCATCCT ACTATATCTA TTTTAAATGT CAATAGATTT TCTTGGCTCT |
| 251 | CCATTTTTTA CAATGGAGAA AGGGGGTTTT GA |

The PSORT algorithm predicts cytoplasm (0.167).

The following C. pneumoniae protein (PID 4376434) was also expressed <SEQ ID 213; cp6434>:

| | |
|---|---|
| 1 | MSESINRSIH LEASTPFFIK LTNLCESRLV KIPSLVISLL ALVGAGVTLV |
| 51 | VLFVAGILPL LPVLILEIIL ITVLVLLWCL VLEPYLIEKP SKIKELPKVD |
| 101 | ELSVVETDST L* |

The cp6434 nucleotide sequence <SEQ ID 214> is:

| | |
|---|---|
| 1 | ATGTCTGAAA GTATTAACAG AAGCATTCAT TTAGAAGCCT CTACACCATT |
| 51 | TTTTATAAAA TTAACGAATC TCTGTGAAAG TAGATTAGTT AAGATCACTT |
| 101 | CTCTTGTTAT TTCTCTATTA GCTTTAGTGG GTGCGGGAGT CACTCTTGTG |
| 151 | GTTTTATTTG TAGCTGGGAT CCTTCCTTTA CTTCCTGTAC TCATCTTAGA |
| 201 | AATTATTTTA ATAACCGTCC TTGTCTTGCT TTTTTGTTTG GTATTGGAAC |
| 251 | CTTATTTAAT AGAAAAACCT AGTAAAATAA AGGAACTACC TAAAGTAGAC |
| 301 | GAGCTATCTG TAGTAGAAAC GGACAGTACT CTTTAA |

The PSORT algorithm predicts inner membrane (0.6859).

The proteins were expressed in E. coli and purified as his-tag products (FIG. 106A; 6306=lanes 24; 6434=lanes 8-10). The recombinant proteins were used to immunise mice, whose sera were used in Western blots (FIGS. 106B & 107) and for FACS analysis.

These experiments show that cp6306 & cp6434 are surface-exposed and immunoaccessible proteins, and that they are useful immunogens. These properties are not evident from the sequences alone.

### Example 108

The following C. pneumoniae protein (PID 4377400) was expressed <SEQ ID 215; cp7400>:

| | |
|---|---|
| 1 | MRVMRFFCLF FLGFLGSFHC VAEDKGVDLF GVWDDNQITE CDDSYMTEGR |
| 51 | EEVEXVVDA |

The cp7400 nucleotide sequence <SEQ ID 216> is:

| | |
|---|---|
| 1 | GTGAGAGTTA TGAGATTTTT TTGTCTATTT TTTCTTGGGT TCCTAGGATC |
| 51 | TTTTCATTGT GTTGCTGAAG ACAAGGGCGT GGATTTATTT GGAGTCTGGG |
| 101 | ACGATAACCA AATTACAGAG TGTGACGATA GTTACATGAC AGAGGGTCGT |

The PSORT algorithm predicts periplasmic space (0.924).

The protein was expressed in E. coli and purified as a GST-fusion product (FIG. 108A). The recombinant protein was used to immunise mice, whose sera were used in a Western blot (FIG. 108B) and for FACS analysis.

These experiments show that cp7400 is a surface-exposed and immunoaccessible protein, and that it is a useful immunogen. These properties are not evident from the sequence alone.

### Example 109

The following C. pneumoniae protein (PID 4376395) was expressed <SEQ ID 217; cp6395>:

| | |
|---|---|
| 1 | MENAMSSSBV YNGPSWILKT SVAQEVPKKH GKGIQVLLST SVMLFIGLGV |
| 51 | CAFIFPQYLI VFVLTIALLM LAISLVLFFL IRSVRSSMVD RLWCSEKGYA |
| 101 | LHQHENGPFL DVKRVQQILL RSPYIKVRAL WPSGDIPEDP SQAAVLLLSP |
| 151 | WTFFSSVDVE ALLPSPQEKE GKYIDPVLPK LSRIERVSLL VFLSAFTLDD |
| 201 | LNEQGVNPIM NNEEFLFFIN KKAREHGIQD LKHEIMSSLE KTGVPLDPSM |
| 251 | SFQVSQAMFS VYRYLRQRDL TTSELRCFHL LSCFKGDVVH CLASFENPKD |
| 301 | LADSDFLEAC KNVEWGEFIS ACEKALLKNP QGISIKDLKQ FLVR* |

The cp6395 nucleotide sequence <SEQ ID 218> is:

| | |
|---|---|
| 1 | ATGGAGAATG CTATGTCATC ATCGTTTGTG TATAATGGGC CTTCGTGGAT |
| 51 | TTTAAAAACG TCAGTAGCTC AGGAGGAATT TAAAAAGCAC GGTAAGGGGA |
| 101 | TTCAGGTTCT CTTAAGTACT TCAGTGATGC TTTTTATAGG TCTTGGAGTC |
| 151 | TGTGCCTTTA TATTTCCTCA ATATCTGATT GTTTTTGTTT TGACTATAGC |
| 201 | TTTGCTTATG CTCGCTATAA GCTTGGTATT GTTTCTCTTA ATACGTTCTG |
| 251 | TACGCTCTTC AATGGTAGAT CGTTTGTGGT GTTCTGAAAA AGGATATGCT |
| 301 | CTTCATCAAC ATGAGAACGG GCCTTTTTTG GATGTGAAGC GTGTACAGCA |
| 351 | AATTCTTCTA AGATCACCCT ATATTAAAGT TCGGGCTTTA TGGCCGTCTG |
| 401 | GAGATATCCC TGAGGATCCT TCACAAGCTG CGGTTCTATT ACTTTCTCCT |
| 451 | TGGACTTTCT TTTCATCCGT GGATGTAGAG GCTTTATTAC CGAGTCCTCA |
| 501 | AGAAAAGGAG GGTAAGTATA TAGATCCTGT GCTGCCTAAG TTGTCTAGGA |
| 551 | TAGAGAGAGT CTCACTTTTA GTGTTTTTGA GTGCATTTAC TTTGGATGAC |
| 601 | TTAAACGAAC AGGGAGTCAA TCCTTTGATG AATAATGAGG AATTTTTATT |
| 651 | TTTTATAAAT AAGAAAGCGC GTGAGCATGG GATTCAGGAT TTAAAACACG |
| 701 | AGATTATGTC TTCGTTAGAG AAAACAGGAG TGCCATTAGA CCCCTCAATG |
| 751 | AGTTTTCAAG TTTCACAAGC GATGTTTTCT GTATATCGCT ACTTGAGACA |
| 801 | AAGGGATTTA ACGACTTCAG AATTAAGATG TTTTCACCTC TTAAGTTGTT |
| 851 | TTAAAGGGGA TGTGGTTCAT TGTTTAGCTT CATTTGAAAA CCCTAAAGAT |
| 901 | TTAGCAGATT CTGACTTTTT AGAAGCTTGT AAGAACGTGG AATGGGGTGA |
| 951 | GTTTATTTCG GCATGTGAGA AGGCTCTTTT AAAGAATCCG CAAGGAATTT |
| 1001 | CCATTAAGGA TCTAAAACAA TTTTTAGTGA GGTAA |

The PSORT algorithm predicts inner membrane (0.6307).

The protein was expressed in E. coli and purified as a GST-fusion product (FIG. 109A). The recombinant protein was used to immunise mice, whose sera were used in a Western blot FIG. 109B) and for FACS analysis.

These experiments show that cp6395 is a surface-exposed and immunoaccessible protein, and that it is a useful immunogen. These properties are not evident from the sequence alone.

### Example 110

The following C. pneumoniae protein (PID 4376396) was expressed <SEQ ID 219; cp6396>:

| | |
|---|---|
| 1 | MIEFAFVPHT SVTADRIEDR MACRMNKLST LAITSLCVLI SSVCIMIGIL |
| 51 | CISGTVGTYA FVVGIIFSVL ALVACVFFLY FFYPSSEEFK CASSQEFRFL |
| 101 | PIPAVVSALR SYEYISQDAI NDVIRDTMQL SThSSLLDPE AFFLEFPYFN |
| 151 | SLIVNUSMKE ADRLSREAFL ILLGEITWXD CETKILPWLK DPNITPDDFW |
| 201 | KLLKDHFDLK DFKKRIATWI RXAYPEIRLP KKHCLDKSIY KGCCKFLLLS |
| 251 | ENDVQYQRLL HKVCYPSGEF PAMVLGLGSE VPMVLGLPKV PKDLTWEMFM |
| 301 | ENMPVLLQSK REGHWKISLE DVASL* |

The cp6396 nucleotide sequence <SEQ ID 220> is:

| | |
|---|---|
| 1 | ATGATCGAGT TTGCTTTTGT TCCTCATACC TCCGTGACAG CGGATCGGAT |
| 51 | TGAGGATCGC ATGGCCTCTC GCATGAACAA GTTGTCTACT TTAGCAATTA |
| 101 | CAAGTCTTTG TGTATTGATC AGTTCAGTTT GTATTATGAT TGGGATTTTA |
| 151 | TGCATTTCTG GAACGGTTGG GACCTATGCA TTTGTTGTAG GAATTATTTT |
| 201 | TTCTGTGCTT GCTTTGGTAG CATGTGTTTT CTTTCTTTAT TTCTTTTATT |
| 251 | TTTCTTCTGA GGAATTTAAG TGTGCTTCTT CGCAGGAGTT TCGTTTTTTG |
| 301 | CCTATACCAG CTGTGGTTTC TGCATTGCGT TCCTATGAAT ACATTTCTCA |
| 351 | GGACGCTATC AATGACGTTA TAAAAGATAC GATGCAGTTG TCTACCCTTP |
| 401 | CTTCTCTTTT AGATCCCGAA GCTTTTTTCT TAGAATPTCC TTATTTTAAC |
| 451 | TCTTTGATAG TGAATCATTC GATGAAGGAA GCGGATCGTT TCTCTCGAGA |
| 501 | GGCTTTTTTG ATTTTATTAG GTGAGATTAC TTGGAAGGAT TGTGAAACAA |
| 551 | AAATTTTGCC ATGGTTGAAA GATCCTAATA TCACTCCTGA TGATTTCTGG |
| 601 | AAGCTAPTAA AAGACCATTT CGATTTAAAG GACTTTAAGA AGAGGATCGC |
| 651 | CACTTGGATA CGGAAGGCCT ATCCAGAAAT TAGATTACCO AAGAAGCATT |
| 701 | GTTTAGATAA GTCTATCTAT AAGGGGTGTT GTAAGTTTTT ATTACTTTCT |
| 751 | GAGAATGATG TGCAATATCA GAGOTTATTA CATAAGGTCT GTTATTTCTC |
| 801 | TGGGGAGTTT CCTGCCATGG TTTTAGGTTT GGGAAGTGAA GTGCCTATGG |
| 851 | TGTTAGGACT CCCTAAGGTT CCCAAGGATC TTACCTGGGA GATGTTTATG |
| 901 | GAAAATATGC CTGTTCTTCT GCAAAGCAAA AGAGAGGGGC ATTGGAAAAT |
| 951 | CTCCTTGGAA GACGTAGCCT CTCTTTAA |

The PSORT algorithm predicts inner membrane (0.6095).

The protein was expressed in E. coli and purified as a GST-fusion product (FIG. 110A). The recombinant protein was used to immunise mice, whose sera were used in a Western blot (FIG. 110B) and for FACS analysis.

These experiments show that cp6396 is a surface-exposed and immunoaccessible protein, and that it is a useful immunogen. These properties are not evident from the sequence alone.

### Example 111

The following C. pneumoniae protein (PID 4376408) was expressed <SEQ ID 221; cp6408>:

| | |
|---|---|
| 1 | MNTfSLKRPLK SHPDVVGSFL RPEHLKRTRE SLKEGSISLD QLMQIEDIAI |
| 51 | QDLIKKQKAA GLSFITDGEF RRATWHYDFM WGFHGVGHHR ATEGVFFPGE |
| 101 | RAMIDDTYLT DKISVSHHPF VDHFKFVKAL EDEFTTAKQT LPAPAQFLKQ |
| 151 | MIFPNNIEVT RXFYPTNQEL IEDIVAGYEK VIRDLYDAGC RYLQLDDCTR |
| 201 | GGLVDPRVCS WYGIDEKGLQ DLIQQYLLIN NLVIADRPDD LVVNLHVCRG |
| 251 | NYHSKFFASG SYDFIAKPLF EQTNVDGYYL EFDHERSGDF SPLTFISGEK |
| 301 | TVCLGLVTSK TPTLENKDEV IARIHQAADY LPLERLSLSP QOOFASCEIG |
| 351 | NKLTEEEQWA KVALVKEISE EVWK* |

The cp6408 nucleotide sequence <SEQ ID 222> is:

| | |
|---|---|
| 1 | ATGAATACTT CACTAAAAAG ACCTCTGAAA TCTCATTTTG ATGTTGTCGG |
| 51 | TAGTTTTTTG CGTCCTGAGC ATTTAAAAAA AACTAGAGAA AGCCTTAAAG |
| 101 | AAGGCTCTAT TTCTCTAGAT CAACTCATGC AAATTGAGGA TATCGCTATC |
| 151 | CAAGATTTGA TCAAAAAACA AAAAGCAGCA GGTCTTTCTT TTATTACTGA |
| 201 | TGGAGAATTC CGCAGAOCTA CGTGGCATTA CGACTTCATG TGGGGTTTTC |
| 251 | ATGGCGTAGG TCACCACAGA GCTACAGAAG GAGTTTTCTT TGATGGAGAA |
| 301 | CGCGCTATGA TCGATGATAC CTATCTGACA GACAAGATCT CTGTATCTCA |
| 351 | CCACCCATTT GTGGATCACT TTAAATTTGT AAAAGCTCTA GAAGATGAAT |
| 401 | TTACCACTGC AAAGCAAACT CTTCCTGCAC CGGCACAGTT TTTAAAGCAX |
| 451 | ATGATCTTCC CTAATAATAT AGAGGTCACA CGTAAATTCT ATCCTACAAA |
| 501 | TCAGGAGCTA ATTGAAGATA TTGTTGCAAG TTATCGTAAA GTCATTCGCG |
| 551 | ATCTTTATGA TGCTGGCTGC CGCTATCTCC AATTAGATGA CTGTACTCGG |
| 601 | GGAGGTTTAG TAGACCCTCG AGTCTGTTCG TGGTATGGTA TCQATGAAAA |
| 651 | AGGTCTTCAA GATCTGATTC AACAATATCT TCTGATTAAT AATCTTGTAA |
| 701 | TTGCAGATCG TCCCGATGAT CTAGTCGTTA ATTTACATGT ATGCCGTGGG |
| 751 | AACTACCACT CAAAATTCTT TGCTAGTGGT AGTTATGACT TTATTGCAAA |
| 801 | GCCCCTATTC GAACAAACAA ATGTAGACGG CTTCTATTTA GAGTTTGATC |
| 851 | ATGAGCGTTC TGGAGACTTC TCTCCTCTCA CCTTCTTTTC TGGAGAAAAA |
| 901 | ACTGTCTGCT TAGGTCTTGT TACCAGCAAA ACCCCTACAC TTGAAAATAA |
| 951 | GGATCAGGTC ATTGCTCGCA TACATCAAGC AGCAGACTAC CTGCCCTTGG |
| 1001 | AAAGACTCTC TCTAAGTCCA CAGTGTGGTT TTGCTTCATG TGAAATAGGA |
| 1051 | AATAAATTAA CAGAAGAAGA GCAATGGGCT AAAGTTGCTC TAGTAAAAGA |
| 1101 | AATTTCCGAA GAAGTTTGGA AATAA |

The PSORT algorithm predicts cytoplasm (0.2171).

The protein was expressed in E. coli and purified as a GST-fusion product (FIG. 111A) and also as a his-tagged product. The his-tag protein was used to immunise mice, whose sera were used in a Western blot (FIG. 111B), and for FACS analysis.

These experiments show that cp6408 is a surface-exposed and immunoaccessible protein, and that it is a useful immunogen. These properties are not evident from the sequence alone.

### Example 112

The following C. pneumoniae protein (PID 4376430) was expressed <SEQ ID 223; cp6430>:

| | |
|---|---|
| 1 | MKLYSISSDV DTPWIFQLMS KVDSYLFLGG NRIKVVSIVN QEPNLIIGKV |
| 51 | ENVRISTIVK ILKILSFLIF PLILIALALH YFLHAKYANH LLVSKILERA |
| 101 | PQYVPXPGRS GDTASHYKLT TLVPVSQKNL QAMGSNPLEV RAMARTTKPS |
| 151 | EECVPAKYRQ IIISSHGIRF SLDLBQLADD INLDSVSWPT EYLNSTMDEC |
| 201 | SKADKRVIQN VQNLRTGTYI NSVGKRSLLK FMLQHLFIDG ITQENPEALP |
| 251 | NNTSGRLTLF PSVRYIYSHF TPQNPTIWPQ VFFRQGPLDE DRGGGFEILB |
| 301 | QLQELGVRPP ICPSQGPDNP NFQGFQGIRX YWEDSYQPNK EV* |

The cp6430 nucleotide sequence <SEQ ID 224> is:

| | |
|---|---|
| 1 | ATGAAACTTT ATAGCATCTC TTCAGATGTA GATACACCTT GGATATTTCA |
| 51 | GCTTATGTCA AAGGTAGATT CTTATCTTTT CTTAGGCGGG AATAGAATCA |
| 101 | AGGTTGTATC TATAGTTATG CAAGAACCTA ACTTAATTAT TGGAAAAGTA |
| 151 | GAAAACGTTC GGNVCTCCAC AATAGTGAAA ATATTAAAAA TTTTATCCTT |
| 201 | CTTAATCTTC CCTCTGATTT TAATCGCTTT AGCCCTACAC TATTTTCTAC |
| 251 | ATGCTAAATA TGCTAATCAC TTACTTGTAT CTAAGATTTT AGAAAGAGCT |
| 301 | CCTCAGTATG TGCCTAATCC TGGTCGTTCA GGAGAQACGG CGTCTCATTA |
| 351 | TAAATTAACA ACATTGGTTC CAGTATCCCA AAAAAATOTA CAAGCPATGG |
| 401 | GATCAAATCC TCTAGAAGTT GAAGCGGCTC TTCGAACTAC AAAACCCTCT |
| 451 | TTTTTCTGTG TACCTGCAAA ATACCGTCAG ATTATAATTT CAAGICACGG |
| 501 | CATTCGCTTT TCTTTAGATC TTGAACAACT TGCTGATGAC ATTAATTTAG |
| 551 | ATTCGGTTTC CTGGCCTACG GAGTATCTTA ACTCTACTAT GGATTTTTGC |
| 601 | AGCAAGGCAG ATAAACGTGT TATACAGAAT GTACAAAATC TGCGGACAGG |
| 651 | AACTTACATA AATTCTGTAG GAAAGCGTAG CCTTTTAAAA TTCATGTTAC |
| 701 | AGCACCTATT TATTGATOGG ATCACACAAG AAAACCCTQA AGCCCTTCCT |
| 751 | AACAATACAT CTGGAAGACT GACTCTATTC CCTAGTGTTC GTTATATCTA |
| 801 | TTCTCATTTT ACTCCACAAA ATCCTACAAT ATGGCCGCAA GTCTTTTTCA |
| 851 | GACAAGGTCC TCTAGATGAA GATCGAGGAG GAGGATTTGA GATCTTAGAG |
| 901 | CAATTACAAG AGTTACGAGT TAGGTTTCCA ATTTGCCCCT CTCAAGGACC |
| 951 | AGACAATCCT AATTTTCAAG GTTTTCAAGG GATTCGTATC TATTGGGAAG |
| 1001 | ATTCCTATCA ACCCAATAAG GAGGTTTAA |

The PSORT algorithm predicts inner membrane (0.5140).

The protein was expressed in E. coli and purified as a GST-fusion product (FIG. 112A). The recombinant protein was used to immunise mice, whose sera were used in a Western blot (FIG. 112B) and for FACS analysis.

These experiments show that cp6430 is a surface-exposed and immunoaccessible protein, and that it is a useful immunogen. These properties are not evident from the sequence alone.

### Example 113

The following C. pneumoniae protein (PID 4376439) was expressed <SEQ ID 225; cp6439>:

| | |
|---|---|
| 1 | MSYDTLFKNL BKEDSVHKIC NEIFALVPRL NTIACTEAII KNLPKADIHV |
| 51 | HLPGTITPQL AWILGVKNGF LKWSYNSWTN HRLLSPKNPH RQYSNIFRNF |
| 101 | QDICHEKDPD LSVLQYNILN YDFNSFDRVM ATVQGHRFPP GGIQNEEDLL |
| 151 | LIFNNYLQQC LDDTIVYTEV QQNIRLAHVL YPSLPEKHAR MKFYQILYRA |
| 201 | SQTFSKHGIT LRFLNCENKT FAPQINTQEP AQEAVQWLQE VDSTFPGLFV |
| 251 | GIQSAGSESA PGACPKRLAS GYENAYDSGF GCEAHAGEGI ETRTIFSSAK |
| 301 | VNPEGLIEIT RVTFSSLKRK QPSSLPIRVT CQLG* |

The cp6439 nucleotide sequence <SEQ ID 226> is:

| | |
|---|---|
| 1 | ATGTCTTATG ATACGTTATT CAAGAATCTT GAAAAGGAAG ATTCTGTACA |
| 51 | TAAGATATGC AATGAGATCT TTGCATTAGT ACCACGACTC AATACAATCG |
| 101 | CTTGCACCGA AGCTATCATC AAAAACCTCC CCAAAGCAGA TATCCATGTA |
| 151 | CACCTTCCTG GGACCATAAC ACCTCAATTA GCTTGGATTT TAGGTGTGAA |
| 201 | AAATGGGTTC TTAAAATGGT CTTATAATTC TTGGACCAAT CATCGATTAC |
| 251 | TTTCTCCPAA GAATCCTCAT AAACAATACT CCAATATTTT CCGAAACTTT |
| 301 | CAAGATATCT GTCACGAAAA GGATCCGGAT TTAAGTGTAT TACAATATAA |
| 351 | TATCTTAAAT TACGATTTTA ATAGCTTTGA TAGAGTGATG GCTACAGTAC |
| 401 | AAGGACATCG CTTTCCTCCT GGTGGAATCC AAAATGAAGA AGACCTTCTT |
| 451 | CTCATTTTCA ATAACTATCT CCAGCAATGT CTGGACGATA CTATCGTGTA |
| 501 | TACTGAAGTA CAACAAAATA TCCGCCTTGC CCATGTTTTG TATCCTTCAT |
| 551 | TACCTGAAAA GCACGCGCGT ATGAAGTTTT ATCAAATCTT GTATCGTGCT |
| 601 | TCGCAAACGT TTTCAAAACA CGGGATTACT TTACGAATTT TAAACTGCTT |
| 651 | CAATAAAACA TTTGCTCCAC AAATTAACAC ACAAGAACCT GCCCAAGAAG |
| 701 | CTGTTCAATG GCTCCAAGAG GTTGATTCTA CATTTCCTGG TCTATTTGTA |
| 751 | GGGATACAAT CCGCAGGATC AGAATCTGCG CCCGGAGCCT GTCCTAAGCG |
| 801 | ATTAGCTTCT GGATATAGAA ATGCTTATGA CTCAGGGTTT GGTIGTGAAG |
| 851 | CTCATGCTGG AGAAGGCATA GAGACCCGGA CTATTTTTTC GTOAGCTTAG |
| 901 | GTAAATCCAG AGGGATTGAT CGAGATAACC CGAGTGACTT TCTCGTCTCT |
| 951 | TAAACGAAAA CAGCCATCTA GTTTACCCAT AAGAGTTACT TGCCAGTTAG |
| 1001 | GATAA |

The PSORT algorithm predicts cytoplasm (0.1628).

The protein was expressed in E. coli and purified as a GST-fusion product (FIG. 113A). The recombinant protein was used to immunise mice, whose sera were used in a Western blot FIG. 113B) and for FACS analysis.

These experiments show that cp6439 is a surface-exposed and immunoaccessible protein, and that it is a useful immunogen. These properties are not evident from the sequence alone.

### Example 114

The following C. pneumoniae protein (PID 4376440) was expressed <SEQ ID 227; cp6440>:

| | |
|---|---|
| 1 | LQSARRHLNT IFILDFGSQY TYVLAKQVRK LFVYCEVLPW NISVQCLKER |
| 51 | APLGIILSGG PHSVYENKAP HLDPEIYKLG IPILAXCYGM QLMARDFGGT |
| 101 | VSPGVGEFGY IPIHLYPCEL FKHIVDCESL ITEIRMSHRD HVTTIPEGFN |
| 151 | VIASTSQCSI SGIENTKQRL YGLQFHPEVS DSTPTGNKIL ETFVQEICSA |
| 201 | PTLWNPLYIQ QDLVSKIQDT VIEVFDEVAQ SLDVQWLAQG TIYSDVIESS |
| 251 | RSGHASEVIK SHHNVGGLPK NLKLKLVEPL RYLPKDEVRI LGEALGLSSY |
| 301 | LLDRHPFPGP GLTIRVIGEI LPEYLAILRR ADLIFIEELR KAKLYDKISQ |
| 351 | AFALFLPIKS VSVKGDCRSY GYTIALRAVE STDFMTGRWA YLPCDVLSSC |
| 401 | SSRIINBIPE VSRVVYDISD KPPATIEWE* |

The cp6440 nucleotide sequence <SEQ ID 228> is:

| | |
|---|---|
| 1 | TTGCAGAGTG CAAGGAGACA TTTGAACACC ATATTTATTC TAGATTTTGG |
| 51 | ATCTCAATAT ACTTATGTAT TAGCAAAGCA AGTGCGGAAG TTATTTGTAT |
| 101 | ATTGCGAAGT TCTTCCCTGG AATATCTCTG TGCAATGTTT AAAAGAAAGA |
| 151 | GCOCCTTTGG GGATCATTCT CTCAGGAGGT CCTCACTCTG TCTATCAAAA |
| 201 | CAAGGCTCCA CATTTATATC CTGAAATCTA TAAACTTGGC ATTCCAATTC |
| 251 | TAGCTATTTG CTATGGCATG CAGCTTATGG CTAGAGATTT TGGAGGGACT |
| 301 | GTAAGCCCPG GTGTAGGAGA ATTTGGATAT ACGCCCATCC ATCTGTATCC |
| 351 | TTGTGAGCTC TTCAAACACA TCGTCGACTG CGAATCTCTA GACACAGAGA |
| 401 | TTCGGATGAG CCATCGGGAT CATGTTACGA CAATTCCTGA AGGATTTAAT |
| 451 | GTAATCGCAT CCACCTCACA ATGCTCGATC TCAGGAATAG AAAATACCAA |
| 501 | ACAACGGTTG ThCGGGCTGC AATTTCATCC CGAGGTTTCT GACTCCACTC |
| 551 | CAACGGGAAA TAAGATTCTA GAAACTTTTG TTCAAGAGAT CTGTTCTGCT |
| 601 | CCCACACTAT GGAATCCCTT GTATATTCAG CAAGACCTTG TAAGTAAAAT |
| 651 | TCAAGATACC GTTATTGAAG TATTTGATGA AGTCGCTCAG TCATTAGACG |
| 701 | TACAATGGTT AGCTCAAGGA ACCATCTACT CAGATGTTAT TGAGTCCTCA |
| 751 | CGCTCTGGAC ATGCCTCCGA AGTAATAAAA TCACATCATA ATGTAGGGGG |
| 801 | GCTTCCAAAA AATCTTAAGC TGAAGTTAGT CGAGCCCTTA CGTTATTTAT |
| 851 | TTAAAGATGA AGTTCGAATT TTAGGAGAAG CCCTAGGACT TTCTAGCTAT |
| 901 | CTCTTGGACA GGCATCCTTT TCCTGGACCT GGCTTGACAA TTCGTGTGAT |
| 951 | TGGAGAGATC CTTCCTGAAT ATCTAGCCAT TTTACGACGG GCGGACCTCA |
| 1001 | TCTTTATAGA AGAGCTTAGG AAAGCAAAAC TCTACGATAA AATAAGCCAA |
| 1051 | GCCTTTGCTC TATTTCTTCC TATAAAATCA QTATCTGTAA AAGGAGATTG |
| 1101 | TAGAAGCTAT GGTTATACCA TAGCATTACG TGCTGTAGAA TCTACAGATT |
| 1151 | TCATGACAGG ACGATGGGCC TACCTTCCAT GCGA1GTTCT CAGTTCTTGC |
| 1201 | TCATCGCGAA TTATTAATGA AATACCCGAG GTAAGCCGAG TGGTCTATGA |
| 1251 | TATTTCTGAC AAGCCACCAG CAACTATAGA ATGGGAATAG |

The PSORT algorithm predicts cytoplasm (0.0481).

The protein was expressed in E. coli and purified as a GST-fusion product (FIG. 114A) and also as a his-tagged product. The recombinant proteins were used to immunise mice, whose sera were used in a Western blot (FIG. 114B) and for FACS analysis.

These experiments show that cp6440 is a surface-exposed and immunoaccessible protein, and that it is a useful immunogen. These properties are not evident from the sequence alone.

### Example 115

The following C. pneumoniae protein (PID 4376475) was expressed <SEQ ID 229; cp6475>:

| | |
|---|---|
| 1 | MNTYTFSPTL QKSFSLFLLE KLDSYFFPGG TRTQILVITP TNIRLAAKKR |
| 51 | GCKVSTIEKI IKILSFILLP IVIIAFILRY FLHKKFDKQF LCIPKVISNE |
| 101 | DEALLGSRPQ AVEKAVREIS PAFFSIPRKY QLIRIDTPKD DAPSILFPIG |
| 151 | IEIILKDLCI DTLKQSNLFL KREMDFLGHP EEKALFDSIC SIEKDQEWMS |
| 201 | LESKKLLITH FLKYLFVSGI EQLNPGFNPE NGRGYFSEIS TAKIHFHQHG |
| 251 | RYGPIRSSGP IMKEI* |

The cp6475 nucleotide sequence <SEQ ID 230> is:

| | |
|---|---|
| 1 | ATGAATACCT ATACCTTCTC TCCTACACTT CAGAAAAGCT TCAGCCTATT |
| 51 | TCTTTTAGAA AAATTAGACT CTTACTTTTT CTTTGGAGGG ACTCGTACAC |
| 101 | AAATCTTAGT CATCACACCA ACCAATATTA GATTAGCAGC TAAAAAAAGA |
| 151 | GGGTGTAAGG TTTCTACTAT AGAAAAGATA ATCAAGATCC TCTCTTTTAT |
| 201 | CCTGCTGCCC CTAGTTATCA TTGCCTTTAT ACTTCGCTAT TTCTTACATA |
| 251 | AGAAATTCGA TAAACAGTTC TTGTGTATCC CAAAAGTCAT TTCTAACGAA |
| 301 | GACGAAGCTC TTCTTGGATC TAGACCACAA GCAGTTGAAA AAGCAGTTCG |
| 351 | AGAAATATCT CCAGCCTTCT TCTCTATACC AAGAAAATAC CAACTTATTA |
| 401 | GAATCGACAC TCCTAAAGAT GACGCTCCCT CAATCCTTTT CCCTATAGGC |
| 451 | ATAGAGATCA TTCTCAAAGA TTTATGTATT GATACACTCA AGCAATCTAA |
| 501 | TCTTTTCCTT AAAAGAGAAA TGGATTTCTT AGGTCATCCA GAAGAAAAAG |
| 551 | CATTATTCGA CTCGATATGT TCTATAGAAA AAGATCAAGA ATGGATGAGC |
| 601 | TTGGAAAGTA AAAAACTTTT AATCACGCAC TTCCTAAAGT ATCTCTTTGT |
| 651 | CTCTGGAATC GAACAACTAA ATCCAGGCTT TAACCCAGAG AATGGGCGTG |
| 701 | GGTATTTTTC AGAAATAAGT ACAGCAAAGA TCCATTTTCA TCAGCACGGT |
| 751 | CGATATGGGC CAATCCGTTC TTCGGGACCC ATCATGAAGG AAATATAA |

The PSORT algorithm predicts inner membrane (0.5373).

The protein was expressed in E. coli and purified as a GST-fusion product (FIG. 115A). The recombinant protein was used to immunise mice, whose sera were used in a Western blot (FIG. 115B) and for FACS analysis.

These experiments show that cp6475 is a surface-exposed and immunoaccessible protein, and that it is a useful immunogen. These properties are not evident from the sequence alone.

### Example 116

The following C. pneumoniae protein (PID 4376482) was expressed <SEQ ID 231;cp6482>:

| | |
|---|---|
| 1 | MLVELEALKR EFAHLKDQKP TSDQEITSLY QCLDHLEFVL LGLGDKFLK |
| 51 | ATEDEDVLFE SQKAIDAWNA LLTKARDVLG LGDIGAIYQT IEFLGAYLSK |
| 101 | VNRRAFCIAS EIHFLKTAIR DLNAYYLLDP RWPLCKIEEF VDWGNDCVEI |
| 151 | AKRKLCTFEK ETKELNESLL REEHAMEKCS IQDLQRKLSD IIIELHDVSL |
| 201 | FCFSKTPSQE EYQKDCLYQS RLRYLLLLYE YTLLCKTSTD FQEQARAKEE |
| 251 | FIREKFSLLE LEKGIKQTKE LEFAIAKSKL ERGCLVMRKY EAAAKHSLDS |
| 301 | MFEEETVKSP RKDTE* |

The cp6482 nucleotide sequence <SEQ ID 232> is:

| | |
|---|---|
| 1 | ATGCTAGTAG AGTTAGAGGC TCTTAAAAGA GAGTTTGCGC ATTTAAAAGA |
| 51 | CCAGAAGCCG ACAAGTGACC AAGAGATCAC TTCACTTTAT CAATGTTTGG |
| 101 | ATCATCTTGA ATTCGTTTTA CTCGGGCTGG GCCAGGACAA ATTTTTAAAG |
| 151 | GCTACGGAAG ATGAAGATGT GCTTTTTGAG TCTCAAAAAG CAATCGATGC |
| 201 | GTGGAATGCT TTATTGACAA AAGCCAGAGA TGTTTTAGGT CTTGGGGACA |
| 251 | TAGGTGCTAT CTATCAGACT ATAGAATTCT TGGGTGCCTA TTTATCAAAA |
| 301 | GTGAATCGGA GGGCTTTTTG TATTGCTTCG GAGATACATT TTCTAAAAAC |
| 351 | AGCAATCCGA GATTTGAATG CATATTACCT GTTAGATTTT AGATGGCCTC |
| 401 | TTTGCAAGAT AGAAGAGTTT GTGGATTGGG GGAATGATTG TGTTGAAATA |
| 451 | GCAAAGAGGA AGCTATGCAC TTTTGAAAAA GAAACCAAGG AGCTCAATGA |
| 501 | GAGCCTTCTT AGAGAGGAGC ATGCGATGGA GAAATGCTCG ATTCAAGATC |
| 551 | TGCAAAGGAA ACTTAGCGAC ATTATTATTG AATTGCATGA TGTTTCTCTT |
| 601 | TTTTGTTTTT CTAAGACTCC CAGTCAAGAG GAGTATCAAA AGGATTGTTT |
| 651 | GTATCAATCA CGATTGAGGT ACTTATTGTT GCTGTATGAG TATACAPTGT |
| 701 | TATGTAAGAC ATCCACAGAT TTTCAAGAGC AGGCTAGGGC TAAAGAGGAG |
| 751 | TTCATTAGGG AGAAATTCAG CCTTCTAGAG CTCGAAAAGG GAATAAAACA |
| 801 | AACTAAAGAG CTTGAGTTTG CAATTGCTAA AAGTAAGTTA GAACGGGGCT |
| 851 | GTTTAGTTAT GAGGAAGTAT GAAGCTGCCG CTAAACATAG TTTAGATTCT |
| 901 | ATGTTCGAAG AAGAAACTGT GAAGTCGCCG CGGAAAGACA CAGAATAA |

The PSORT algorithm predicts cytoplasm (0.4607).

The protein was expressed in E. coli and purified as a GST-fusion product (FIG. 116A). The recombinant protein was used to immunise mice, whose sera were used in a Western blot (FIG. 116B) and for FACS analysis.

These experiments show that cp6482 is a surface-exposed and immunoaccessible protein, and that it is a useful immunogen. These properties are not evident from the sequence alone.

### Example 117

The following C. pneumoniae protein (PID 4376486) was expressed <SEQ ID 233; cp6486>:

| | |
|---|---|
| 1 | VVVVALFILG IFFLSGSLAF LVHTSCGVLL GAALPILCIG LVLLAVALIV |
| 51 | FLCHKHKTRQ DLDYYDQDLD SLVIHKKEIP NDISELRVTF EKLQNLFQFH |
| 101 | TKDFSDLSQE LQGKFINCME KWLTLEDEVT KFLIVRDRFL ETRRNFTTFG |
| 151 | EQVKGIQSNI FDLHEEKSSL YLELYRLRKD LQVLLNFFLL PPGILKVDYD |
| 201 | EIEAIKGLFI RLTSRLDKLD VKAQERKKFI NEMSREFKEV EKAFDIVDRA |
| 251 | TKKLMDRAKK ESPARLFMGR TESLLEMKKN EEALKNQGLD PENLSHPELF |
| 301 | SPYQQLLILN YLNSEIVLHH YEFLISGTVT SGLTLEECEN RMRAASTGLN |
| 351 | ALLVRKLQFR GAIKSAYFEK LTEIEKELRS LQDVIKSLEL ELIKHIKDIV |
| 401 | TEET* |

The cp6486 nucleotide sequence <SEQ ID 234> is:

| | |
|---|---|
| 1 | GTGGTGGTTG TCGCTTTATT TATCCTTGGG ATTTTCTTTT TATCTGGTTC |
| 51 | TCTTGCATTC CTTGTTCATA CGTCTTGCGG AGTTCTTTTA GGAGCGGCGC |
| 101 | TTCCCATACT TTGCATAGGT CTTGTTTTAT TGGCTGTAGC TCTTATTGTT |
| 151 | TTCTTATGTC ACAAACACAA GACTCGTCAA GATTTAGATT ATTATGATCA |
| 201 | AGATTTAGAT TCTTTGGTGA TTCATAAGAA AGAGATCCCC AATGACATCT |
| 251 | CTGAGTTGCG GGTAACATTT GAAAAGTTGC AAAATCTGTT TCAGTTCCAT |
| 301 | ACGAAAGATT TCTCTGATCT AAGCCAAGAG CTTCAGGGTA AATTTATCAA |
| 351 | TTGCATGGAG AAATGGCTAA CTTTAGAAGA CGAAGTGACT AAATTTCTTA |
| 401 | TTGTTCGAGA TAGATTTTTA GAAACCAGAA GAAATTTTAC CACTTTTGGA |
| 451 | GAACAGGTTA AAGGGATCCA AAGCAATATT TTTGATTTGC ATGAGGAAAA |
| 501 | GTCTTCATTA TATTTAGAAT TGTATAGGCT TAGGAAAGAC CTCCAAGTTC |
| 551 | TATTAAATTT TTTTCTGCTC CCCCCAGGTA TACTCAAGGT AGATTATGAT |
| 601 | GAAATTGAGG CTATCAAAGG TCTGTTTATA AGATTAACCT CTAGATTAGA |
| 651 | TAAGCTTGAT GTGAAAGCTC AGGAACGTAA GAAGTTCATT AATGAAATGA |
| 701 | GTAGGGAATT TAAAGAAGTA GAGAAAGCTT TTGATATTGT CGATAGGGCA |
| 751 | ACAAAAAAGC TTATGGATAG AGCCAAGAAA GAAAGTCCGG CACGTCTTTT |
| 801 | CATGGGTAGA ACTGAGTCTC TCTTAGAAAT GAAAAAAAAT GAAGAAGCCC |
| 851 | TTAAAAATCA GGGGCTAGAT CCTGAAAATC TTTCCCATCC TGAACTTTTT |
| 901 | AGTCCGTATC AACAGCTTTT AATTTTGAAT TATTTAAATA GCGAAATAGT |
| 951 | TCTGCATCAT TATGAGTTCC TTATTTCTGG AACAGTAACT TCTGGCCTAA |
| 1001 | CTCTTGAAGA ATGTGAAAAT CGAATGAGGG CGGCTTCTAC TGGGTTGAAC |
| 1051 | GCCCTTCTGG TGCGTAAGCT CCAGTTCAGA GGTGCTATAA AATCTGCGTA |
| 1101 | TTTTGAAAAA CTCACAGAGA TTGAAAAAGA GTTACGATCA CTTCAAGACG |
| 1151 | TAATAAAGTC ATTGGAACTA GAACTGATCC ATAAGATAAA AGATATAGTG |
| 1201 | ACAGAAGAAA CTTAG |

The PSORT algorithm predicts inner membrane (0.7474).

The protein was expressed in E. coli and purified as a GST-fusion product (FIG. 117A). The recombinant protein was used to immunise mice, whose sera were used in a Western blot (FIG. 117B) and for FACS analysis.

These experiments show that cp6486 is a surface-exposed and immunoaccessible protein, and that it is a useful immunogen. These properties are not evident from the sequence alone.

### Example 118

The following C. pneumoniae protein (PID 4376526) was expressed <SEQ ID 235; cp6526>:

| | |
|---|---|
| 1 | MSPFKKIVNR LLCYISFQKE SRTLPIIIRE PRMTTKSLGS FNSVISKNKI |
| 51 | HFISLGCSRN LVDSEVMLGI LLKAGYESTN EIEDADYLIL NTCAFLKSAR |
| 101 | DEAKDYLDHL IDVKKENAKI IVTGCMTSNH KDELKPWMSH IHYLLGSGDV |
| 151 | ENILSAIESR ESGEKISAKS YIEMGEVPRQ LSTPKHYAYL KVAEGCRKRC |
| 201 | AFCIIPSIKG KLRSKPLDQI LKEFRILVNK SVKEIILIAQ DLQDYGKDLS |
| 251 | TDRSSQLESL LHELLKEPGP YWLRMLYLYP DEVSDGIIDL MQSNPKLLPY |
| 301 | VDIPLQHIND RILKQMRRTT SREQILGFLE KLRAKVPQVY IRSSVIVGFP |
| 351 | GETQEEFQEL ADFIGEGWID NLGIFLYSQE ANTPAAELPD QIPEKVKESR |
| 401 | LKILSQIQKR NVDKHNQKLI GEKIEAVIDN YHPETNLLLT ARFYGQAPEV |
| 451 | DPCIIVNEAK LVSHFGERCF IEITGTAGYD LVGRVVKKSQ NQALLKTSKA |
| 501 | * |

The cp6526 nucleotide sequence <SEQ ID 236> is:

| | |
|---|---|
| 1 | ATGAGTCCTT TTAAGAAAAT AGTAAATCGC TTACTATGCT ATATTTCTTT |
| 51 | TCAAAAAGAA TCAAGAACTC TCCCAATCAT TATTAGAGAA CCTAGGATGA |
| 101 | CAACAAAAAG TTTAGGATCT TTCAATTCAG TTATTTCCAA AAATAAAATT |
| 151 | CATTTTATTA GTTTGGGATG CTCTCGGAAC CTTGTAGATA GCGAAGTCAT |
| 201 | GCTAGGCATT CTTCTTAAGG CAGGTTACGA GTCTACTAAT GAAATTGAAG |
| 251 | ATGCTGACTA TTTAATTTTA AATACCTGTG CGTTTTTAAA AAGTGCTAGA |
| 301 | GATGAAGCTA AAGATTATCT AGACCATCTA ATTGATGTAA AAAAAGAGAA |
| 351 | CGCTAAAATT ATTGTAACTG GATGCATGAC TTCCAACCAC AAAGATGAGC |
| 401 | TTAAACCCTG GATGTCACAC ATCCATTACC TACTAGGTTC TGGGGATGTT |
| 451 | GAGAATATTC TTTCTGCTAT TGAGTCTCGT GAATCTGGAG AAAAAATCTC |
| 501 | TGCAAAGAGT TACATTGAGA TGGGAGAAGT TCCAAGACAG CTTTCCACAC |
| 551 | CAAAACACTA TGCCTATTTA AAAGTTGCTG AGGGCTGTAG AAAACGTTGT |
| 601 | GCTTTTTGTA TTATTCCTTC CATTAAAGGA AAGCTCCGCA GCAAACCTCT |
| 651 | GGATCAAATT CTTAAAGAAT TCCGCATCCT TGTAAACAAG AGTGTGAAAG |
| 701 | AGATTATATT GATAGCTCAA GACCTAGGAG ATTATGGAAA GGATCTCTCT |
| 751 | AGAGACCGCA GTTCGCAGCT AGAATCACTA TTACATGAGT TACTGAAAGA |
| 801 | GCCTGGTGAT TATTGGCTGC GGATGTTGTA TTTATATCCT GATGAAGTGA |
| 851 | GTGATGCCAT TATAGATCTT ATGCAATCTA ATCCCAAACT TCTTCCCTAT |
| 901 | GTAGATATTC CCTTACAGCA CATTAACGAC CGTATTTTAA AGCAAATGCG |
| 951 | AAGAACGACT TCTAGGGAGC AAATCCTAGG ATTCCTAGAA AAATTACGTG |
| 1001 | CCAAGGTTCC TCAGGTCTAT ATCCGTTCTT CTGTTATTGT GGGTTTCCCC |
| 1051 | GGTGAAACTC AGGAAGAATT CCAGGAGTTA GCTGATTTTA TTGGTGAGGG |
| 1101 | TTGGATTGAT AATCTCGGAA TTTTCTTGTA CTCTCAAGAA GCGAATACCC |
| 1151 | CGGCAGCAGA ACTCCCTGAC CAGATACCAG AAAAAGTTAA AGAATCGAGG |
| 1201 | TTGAAAATTC TATCTCAAAT TCAGAAACGC AATGTGGATA AACATAATCA |
| 1251 | GAAGCTCATT GGGGAAAAAA TAGAAGCAGT TATTGATAAC TATCATCCTG |
| 1301 | AAACGAATCT TTTACTCACT GCAAGGTTCT ATGGACAAGC TCCTGAAGTG |
| 1351 | GACCCTTGTA TTATTGTAAA TGAGGCGAAG CTTGTTTCTC ATTTTGGAGA |
| 1401 | AAGATGCTTT ATAGAAATCA CAGGGACTGC TGGTTACGAC CTTGTAGGGC |
| 1451 | GTGTTGTAAA AAAATCTCAG AACCAAGCTT TGCTAAAAAC TAGCAAAGCT |
| 1501 | TAG |

The PSORT algorithm predicts cytoplasm (0.1296).

The protein was expressed in E. coli and purified as a GST-fusion product (FIG. 118A) and also as a his-tagged product. The recombinant proteins were used to immunise mice, whose sera were used in a Western blot (FIG. 118B) and for FACS analysis.

These experiments show that cp6526 is a surface-exposed and immunoaccessible protein, and that it is a useful immunogen. These properties are not evident from the sequence alone.

### Example 119

The following C. pneumoniae protein (PID 4376528) was expressed <SEQ ID 237; cp6528>:

| | |
|---|---|
| 1 | MKNNINNNEC YFKLDSTVDG DLLAANLKTF DTQAQGISST ETFSVQGNAT |
| 51 | FKDQVSATGL TSGTTYNLNA QNFTSSQISI DFKNNRLSNC ALPKEDCDPV |
| 101 | PANYVRSPEY FFCSKPLIGD FDFNSGESYL PLTGSEYTLY QSRNVNSIFR |
| 151 | FIGWKQSTRE LTVGGNTAIQ FLAAGTYIVS FTVGKRWGWN NGWGGAIYIN |
| 201 | NGLGQVQCES TIYSGGGYAT IGTLGTSIYR ASVDVAPNPN DPNASDRYRA |
| 251 | GIFYLSNGGS SAGIGNYSFS LLYYPDDRG* |

The cp6528 nucleotide sequence <SEQ ID 238> is:

| | |
|---|---|
| 1 | ATGAAAAACA ATATTAATAA TAATGAGTGC TATTTTAAAT TAGACTCAAC |
| 51 | TGTAGATGGT GATTTGTTAG CAGCCAATCT CAAGACCTTT GATACACAGG |
| 101 | CCCAAGGAAT CTCATCGACT GAAACATTTT CTGTTCAGGG GAATGCAACA |
| 151 | TTTAAAGATC AAGTTTCAGC AACTGGATTA ACTTCAGGAA CTACTTATAA |
| 201 | TTTAAATGCA CAAAACTTTA CTTCCTCCCA AATCTCTATA GATTTTAAAA |
| 251 | ATAATCGTCT GAGTAATTGT GCATTGCCAA AAGAAGACTG CGATCCGGTG |
| 301 | CCAGCGAATT ATGTTCGTTC TCCCGAATAT TTTTTCTGTT CCAAGCCTCT |
| 351 | GATCGGAGAT TTTGATTTTA ACTCAGGGGA ATCTTATTTG CCTCTGACTG |
| 401 | GTTCGGAATA TACTCTATAT CAGTCACGTA ATGTAAATAG TATATTTCGT |
| 451 | TTTATAGGAT GGAAGCAAAG TACACGAGAA TTAACTGTAG GGGGAAATAC |
| 501 | TGCGATACAA TTTCTTGCAG CAGGAACCTA TATCGTTTCA TTTACTGTTG |
| 551 | GTAAACGGTG GGGATGGAAT AATGGTTGGG GAGGAGCCAT TTATATCAAT |
| 601 | AATGGTTTAG GACAAGTCCA ATGTGAAAGC ACGATTTATA GTGGTGGAGG |
| 651 | GTATGCAACA ATAGGTACAC TGGGGACCTC AATATATAGA GCCTCTGTAG |
| 701 | ATGTAGCTCC TAATCCTAAT GATCCGAATG CTTCGGATCG CTATAGAGCG |
| 751 | GGTATTTTCT ATCTCAGTAA CGGTGGTTCT AGTGCAGGTA TAGGGAATTA |
| 801 | CTCCTTTTCT CTTCTCTATT ATCCGGACGA TAGAGGGTAG |

The PSORT algorithm predicts cytoplasm (0.1668).

The protein was expressed in E. coli and purified as a GST-fusion product (FIG. 119A). The recombinant protein was used to immunise mice, whose sera were used in a Western blot (FIG. 119B) and for FACS analysis.

These experiments show that cp6528 is a surface-exposed and immunoaccessible protein, and that it is a useful immunogen. These properties are not evident from the sequence alone.

### Example 120

The following C. pneumoniae protein (PID 43376627) was expressed <SEQ ID 239; cp6627>:

| | |
|---|---|
| 1 | MKCSPLTLVP HIFLKNDCEC HRSCSLKIRT IARLILGLVL ALVSALSFVF |
| 51 | LAAPISYAIG GTLALAAIVI LIITLVVALL AKSKVLPIPN ELQKIIYNRY |
| 101 | PKEVFYFVKT HSLTVNELKI FINCWKSGTD LPPNLHKKAE AFGIDILKSI |
| 151 | DLTLFPEFEE ILLQNCPLYW LSHFIDKTES VAGEIGLNKT QKVYGLLGPL |
| 201 | AFHKGYTTIF HSYTRPLLTL ISESQYKFLY SKASKNQWDS PSVKKTCEEI |
| 251 | FKELPHNMIF RKDVQGISQF LFLFFSHGIT WEQAQMIQLI NPDNWKMLCQ |
| 301 | FDKAGGHCSM ATFGGFLNTE TNMFDPVSSN YEPTVNFMTW KELKVLLEKV |
| 351 | KESPMHPASA LVQKICVNTT HHQNLLKRWQ FVRNTSSQWT SSLPQYAFHA |
| 401 | QTYKLEKKIE SSLPIRSSL* |

The cp6627 nucleotide sequence <SEQ ID 240> is:

| | |
|---|---|
| 1 | ATGAAGTGTA GTCCTTTAAC ACTAGTTCCC CATATATTTT TAAAAAATGA |
| 51 | CTGCGAATGT CATAGATCTT GTTCTTTAAA AATTAGGACA ATTGCCCGAC |
| 101 | TCATTCTTGG GCTTGTTCTA GCTCTTGTTA GCGCACTTTC TTTTGTTTTC |
| 151 | CTTGCTGCGC CGATTAGCTA TGCTATTGGA GGAACTTTAG CTTTAGCCGC |
| 201 | TATCGTAATC TTGATTATAA CGCTAGTCGT AGCACTGCTA GCTAAATCAA |
| 251 | AGGTTCTGCC CATCCCCAAC GAACTTCAGA AGATTATTTA CAATCGCTAT |
| 301 | CCTAAAGAAG TCTTTTATTT CGTGAAAACA CACTCCCTGA CTGTTAACGA |
| 351 | ATTAAAAATA TTTATTAATT GCTGGAAAAG CGGTATAGAC CTGCCTCCGA |
| 401 | ATTTACATAA AAAAGCAGAG GCTTTCCGGA TCGATATTCT AAAATCTATA |
| 451 | GATTTAACCC TGTTTCCAQA GTTCGAAGAG ATTCTTCTTC AAAACTGCCC |
| 501 | GTTATACTGG CTCTCCCATT TTATAGACAA AACTGAATCT GTTGCTGGGG |
| 551 | AAATCGGATT AAATAAAACA CAAAAAGTTT ATGGTTTACT TGGGCCCTTA |
| 601 | GCGTTTCATA AAGGATATAC AACTATTTTC CACTCTTATA CACGCCCTCT |
| 651 | ACTAACATTA ATCTCAGAAT CACAGTATAA GTTCCTATAT AGTAAAGCGT |
| 701 | CTAAGAATCA ATGGGATTCT CCTTCTGTGA AAAAAACCTG CGAAGAAATA |
| 751 | TTCAAGGAAC TCCCCCACAA TATGATTTTC CGGAAGGATG TTCAAGGAAT |
| 801 | CTCACAATTC TTATTTCTTT TCTTTTCTCA TGGTATCACT TGGGAACAGG |
| 851 | CTCAGATGAT TCAACTTATA AATCCTGATA ATTGGAAAAT GTTGTGTCAG |
| 901 | TTTGATAAAG CAGGAGGOCA CTGTTCCATG GCAACATTTG GAGGCTTTTT |
| 951 | GAATACTGAA ACAAATATGT TCGATCCAGT ATCCTCTAAC TATGAACCTA |
| 1001 | CAGTGAACTT CATGACGTGG AAAGAATTGA AGGTTTTACT AGAGAAAGTA |
| 1051 | AAAGAAAGTC CTATGCACCC AGCGAGTGCT CTTGTTCAGA AGATATGCGT |
| 1101 | AAATACAACG CACCATCAAA ATCTGTTAAA ACGATGGCAA TTTGTTCGTA |
| 1151 | ATACGAGTTC ACAATGGACA TCAAGCTTAC CTCAGTATGC TTTCCACGCC |
| 1201 | CAAACCTACA AACTAGAGAA AAAAATAGAA AGCAGTCTCC CTATACGATC |
| 1251 | TTCCCTATAA |

The PSORT algorithm predicts inner membrane (0.7198).

The protein was expressed in E. coli and purified as a GST-fusion product (FIG. 120A). The recombinant protein was used to immunise mice, whose sera were used in a Western blot (FIG. 120B) and for FACS analysis.

These experiments show that cp6627 is a surface-exposed and immunoaccessible protein, and that it is a useful immunogen. These properties are not evident from the sequence alone.

### Example 121

The following C. pneumoniae protein (PID 4376629) was expressed <SEQ ID 241; cp6629>:

| | |
|---|---|
| 1 | MSNITSPVIQ NNRSCNYYFE LKNSTTIHIV ISAILLCGAL IAFLCVAAPV |
| 51 | SYILSGALLG LGLLIALIGV ILGIKKITPM ISSKEQVFPQ ELVNRIRAHY |
| 101 | PKFVSDFVSE AKPNLKDLIS FIDLLNQLHS EVGSSTNYNV SEELQQKIDT |
| 151 | FEGIARLKNE VRTASLKRLE SAASSRPLFP SLPKXLQKVF PFFWLGEFIS |
| 201 | AGSKVVELHR VKKIGGSLEE DLSDYIKPEM LPTYWLIPLD FRPTNSSILN |
| 251 | LHTLVLARVL TRDVFQHLKY AALNGEWNLN HSDLNTMCQQ LFAKYHAAYQ |
| 301 | SYKHLSQPSL QEDEFYNLLL CIFKHRYSWK QMSLIKTVPA DLWENLCCLT |
| 351 | LDHTGRPQDM EFASLIGTLY TQGLIHKESE AFLSSLTLLS LDQFKTIRRQ |
| 401 | STNIAMFLEN LATHNSTFRS LPPITVHPLK RSVFSQPEED ESSLLIG* |

The cp6629 nucleotide sequence <SEQ ID 242> is:

| | |
|---|---|
| 1 | ATGAGTAATA TAACCTCGCC AGTTATTCAA AATAATCGCT CTTGTAATTA |
| 51 | TTATTTTGAA TTAAAGAATT CAACCACTAT TCATATTGTT ATCAGTGCCA |
| 101 | TCTTACTCTG CGGAGCTTTG ATAGCTTTCT TGTGTGTAGC AGCTCCTGTT |
| 151 | TCCTATATTC TAAGTGGCGC ATTGTTAGGA TTAGGATTAT TAATAGCCTT |
| 201 | GATTGGTGTG ATTTTAGGAA TAAAAAAAAT CACGCCTATG ATTTCATOAA |
| 251 | AAGAACAAGT ATTCCCCCAA GAACPCGTAA ATAGAATCAG GGCGCACTAT |
| 301 | CCTAAATTTG TCTCTGATTT TGTTTCAGAA GCTAAACCAA ATCTTAAAGA |
| 351 | TCTCATAAGT TTTATTGATC TTCTAAATCA ATTGCACTCT GAAGTTGGAT |
| 401 | CATCTACAAA TTACAACGTA TCTGAAGAAC TACAACAGAA AATAGATACG |
| 451 | TTCGAGGGTA TCGCACQCTT AAAAAATGAA GTCCGTACTG CTTCTCTTAA |
| 501 | AAGACTTGAA AGCGCTGCTT CTTCCCGTCC CCTCTTCCCC TCTTTACCAA |
| 551 | AAATCTTACA AAAGGTATTT CCATTTTTCT GGTTAGGAGA GTTTATTTCT |
| 601 | GCAGGCAGCA AGGTTGTAGA GCTCCATCGA GTTAAGAAAA TTGGAGGCAG |
| 651 | CCTCGAAGAA GACCTTAGTG ATTATATAAA ACCAGAGATG CTTCCTACCT |
| 701 | ATTGGTTGAT TCCTTTAGAT TTTAGACCAA CAAATTCCTC TATTCTAAAT |
| 751 | CTACACACAT TAGTTTTAGC TAGAGTCTTA ACTCGTGATG TTTTTCAACA |
| 801 | TCTTAAGTAT GCAGCATTAA ATGGCGAGTG GAACCTGAAT CATAGTGATC |
| 851 | TAAATACTAT GAAACAGCAG CTCTTTGCTA AATATCATGC GGCGTATCAA |
| 901 | TCCTATAAAC ATCTATCTCA ACCCTCTCTT CAAGAGGATG AATTCTATAA |
| 951 | CCTGCTCTTG TGTATTTTTA AGCATAGGTA CTCGTGGAAG CAGATGTCCT |
| 1001 | TAATAAAAAC AGTCCCGGCT GATTTATGGG AAAACCTCTG TTGCTTGACT |
| 1051 | TTAGACCATA CAGGACGACC CCAAGACATG GAATTTGCCT CTCTAATTGG |
| 1101 | TACTCTCTAC ACACAAGGCC TAATTCATAA AGAAAGCGAA GCATTTCTTT |
| 1151 | CTTCATTGAC ACTCCTTAGT TTAGATCAGT TTAAAACGAT CCGTCGTCAG |
| 1201 | TCAACCAATA TAGCGATGTT CCTTGAGAAT TTAGCAACTC ATAATTCCAC |
| 1251 | CTTTAGAAGC TTACCACCTA TAACAGTCCA TCCACTCAAG AGAAGCGTCT |
| 1301 | TCTCCCAACC TGAAGAAGAC GAGTCCTCCC TGCTGATAGG TTAG |

The PSORT algorithm predicts inner membrane (0.5776).

The protein was expressed in E. coli and purified as a GST-fusion product (FIG. 121A). The recombinant protein was used to immunise mice, whose sera were used in a Western blot (FIG. 121B) and for FACS analysis.

These experiments show that cp6629 is a surface-exposed and immunoaccessible protein, and that it is a useful immunogen. These properties are not evident from the sequence alone.

### Example 122

The following C. pneumoniae protein (PID 4376732) was expressed <SEQ ID 243; cp6732>:

| | |
|---|---|
| 1 | MEMMSPFQQP EQCHFDVVGS FLRPESLTRA RSDGEEGRIV YEQMRVVEDA |
| 51 | AIRNLIKKQT EAGLIFFTDG EFRRYSWDFW FMWGFHGVDR RRDSNDPEIG |
| 101 | VYLKDKISVS KHPFIEHFEF VKTFEKGNAK AKQTIPSPSQ FFHEMIFAPN |
| 151 | LKNTRKFYPT NQELIDDIVF YYRQVIQDLY AAGCRNLQLD DCAWCRLLDI |
| 201 | RAPSWYGVDS HDRLQEILEQ FLWIHNLVMK DRPEDLFVSL HVCRGDYQAE |
| 251 | FFSRRAYDSI EEPLFAKTDV DSYHYYWALD DKYSGGAEPL AYVSGEKHVC |
| 301 | LGLISSNHSC IEDRDAVVSR IYEAASYIPL ERLSLSPQCG FASCEGDHRM |

The cp6732 nucleotide sequence <SEQ ID 244> is:

| | |
|---|---|
| 1 | ATGGAAATGA TGAGCCCATT CCAACAACCT GAGCAATGTC ATTTTGATGT |
| 51 | TCTGGGAAGT TTCTTACGTC CTGAAAGTCT TACACGAGCA CGCTCTGATT |
| 101 | TTGAAGAAGG AAGAATTGTC TATGAGCAGA TGCGAGTTGT CGAAGATGCT |
| 151 | GCTATTCGTA ATCTCATAAA AAAGCAAACA GAAGCAGGTC TTATCTTTTT |
| 201 | TACTGATGGG GAATTCCGTA GGTATAGTTG GGATTTCGAC TTTATGTGGG |
| 251 | GATTCCATGG CGTGGATCGT CGCAGGGACT CTAATGACCC TGAAATTGGA |
| 301 | GTGTATCTTA AAGATAAAAT CTCCGTATCA AAACATCCGT TTATAGAACA |
| 351 | TTTCGAGTTT GTCAAAACTT TTGAGAAGGG AAATGCAAAA GCAAAACAAA |
| 401 | CGATTCCTTC TCCATCACAA TTTTTCCATG AGATGATTTT TGCTCCTAAT |
| 451 | CTGAAAAATA CTCGGAAGTT TTATCCTACG AATCAAGAGC TAATTGATGA |
| 501 | TATTGTCTTT TATTATCGCC AAGTCATCCA AGATCTTTAT GCTGCAGGTT |
| 551 | GTCGTAATTT GCAGTTGGAC GATTGTGCTT GGTGTCGCCT CTTGGATATA |
| 601 | CGAGCGCCTT CTTGGTATGG TGTTGATTCT CATGACAGGT TGCAGGAAAT |
| 651 | TTTAGAACAG TTTTTATGGA TCCATAATTT AGTGATGAAG GATAGACCCG |
| 701 | AGGATCTTTT TGTAAGTCTG CATGTCTGTC GTGGTGATTA TCAGGCCGAG |
| 751 | TTTTTCTCTA GACGAGCTTA TGATTCTATA GAGGAGCCTT TATTTGCTAA |
| 801 | GACCGATGTG GATAGTTATC ACTATTATTG GGCTCTTGAT GATAAGTATT |
| 851 | CAGGAGGTGC TGAGCCTTTA GCTTACGTCT CTGGAGAGAA ACACGPCTGC |
| 901 | TTGGGATTGA TCTCCAGCAA CCATTCTTGT ATTGAAGATC GAGATGCTGT |
| 951 | GGTTTCTCGT ATTTATGAAG CTGCGAGCTA CATTCCCTTA GAGAGACTTT |
| 1001 | CTTTGAGCCC GCAATGTGGG TTTGCTTCTT GTGAGGGAGA CCATAGAATG |
| 1051 | ACTGAAGAAG AACAGTGGAA GAAGATCGCC TTTGTGAAAG AGATTGCTAA |
| 1101 | AGAGATCTGG GGATAA |

The PSORT algorithm predicts cytoplasm (0.2196).

The protein was expressed in E. coli and purified as a GST-fusion product (FIG. 122A). The recombinant protein was used to immunise mice, whose sera were used in a Western blot (FIG. 122B) and for FACS analysis.

These experiments show that cp6732 is a surface-exposed and immunoaccessible protein, and that it is a useful immunogen. These properties are not evident from the sequence alone.

### Example 123

The following C. pneumoniae protein (PID 4376738) was expressed <SEQ ID 245; cp6738>:

| | |
|---|---|
| 1 | VWLRFLLLVS YDEKEKDVVV VCNHSEPNIL GLPPEAVSQL IEELSDEGYS |
| 51 | YLNVVRCDLS GETTVQQRLL LNADEGRSMT VVISELPEGH PDIRNLQLAS |
| 101 | ERIFVSREKE AADAYASGCK VVAFDDEHLP WVSSHIAYAE EIREKQEQTM |
| 151 | QGSLTEEQLG ALLCNTVSTE KNLAFALDAV IKQSVWRFRN PDLFAYEREA |
| 201 | LEASVTDALV SYVSNLDMIP YTSSQGIVIE DSSIVRTSQE HTLXVNCAAF |
| 251 | DKLASQIEFL CPSDVLPISG KDPLISDDED EELNPKVSSA ADSKDKT* |

The cp6738 nucleotide sequence <SEQ ID 246> is:

| | |
|---|---|
| 1 | GTGTGGCTGC GCTTTTTACT TTTAGTGTCC TATGATGAGA AGGAGAAAGA |
| 51 | CGTAGTTGTC GTTTGTAATC ATTCTGAACC TAATATCCTC GGCCTGCCTC |
| 101 | CTGAAGCAGT CTCTCAGCTT ATTGAAGAGC TTAGCGATGA AGGCTATAGC |
| 151 | TATCTGAATG TAGTGCGTTG TGATCTCTCC GOGGACACTA CGGTTCAACA |
| 201 | ACGTCTGCTA TTGAATGCCG ATGAAGGGAG ATCTATGACG GTGGTGATCT |
| 251 | CAGAGCTTCC TGAAGGGCAC CCCGATATTC GGAATTTGCA GTTGGCATCC |
| 301 | GAAAGAATTT TTGTTTCTCG TGAAAAAGAA GCTGCTGATG CCTATGCTTC |
| 351 | AGGATGTAAA GTGGTCGCTT TCGATQATGA GCATCTCCCT TGGGTOTCCA |
| 401 | GTCATATTGC CTACGCGGAG GAGATCAGAG AGAAACAAGA ACAAACAATG |
| 451 | CAAGGGTCTT TAACTGAAGA GCAGTTAGGA GCACTCCTCT GCAACACAGT |
| 501 | CTCCACAGAG AAAAATCTAG CCTTTGCTTP AGACGCCGTG ATAAAACAGT |
| 551 | CTGTGTGGAG ATTCCGCAAT CCGGATCTTT TTGCTTATGA GAGAGAAGCT |
| 601 | CTAGAGGCTT CAGTAACAGA TGCTTTAGTA TCTTACGTTT CAAATTTAGA |
| 651 | CATGATACCG TACACAAGTT CTCAGGGCAT AGTCATAGAA GATAGTAGTA |
| 701 | TCGTCCGTAC CTCTCAAGAG CATACACTCA TTGTGAACTG TGCAGCATTC |
| 751 | GATAAGTTAG CGAGCCAAAT AGAGTTCTTA TGCCCCAGTG ACGTGTTGCC |
| 801 | CATTTCTGGT AAAGACCCTT TGATTTCTGA TGATGAGGAT GAGGAACTGA |
| 851 | ATCCTAAAGT TTCATCTGCT GCAGACTCTA AAGATAAAAC CTAG |

The PSORT algorithm predicts cytoplasm (0.1587).

The protein was expressed in E. coli and purified as a GST-fusion product (FIG. 123A). The recombinant protein was used to immunise mice, whose sera were used in a Western blot (FIG. 123B) and for FACS analysis.

These experiments show that cp6738 is a surface-exposed and immunoaccessible protein, and that it is a useful immunogen. These properties are not evident from the sequence alone.

### Example 124

The following C. pneumoniae protein (PID 4376739) was expressed <SEQ ID 247; cp6739>:

| | |
|---|---|
| 1 | MTHCLHGWFS VVRHHFVQAF NFSRPLYSRI THFALGVIKA IPIVGHLVMG |
| 51 | VDWLISHCFE RGVSHPGFPS DIAPILKVEK IAGRDHISRI ENQLKSLRKT |
| 101 | IEVEDLDKVH GQYQENPYAD MASSEVLKLD KGVHVSELGK AFSRVRNRIT |
| 151 | RSYSYAPTPQ LDSIAIVGID LVSPEEQENI VRLANEVIQL YPKSKTTLYL |
| 201 | LIDFNKEWVG DISSDKEKQL RSLGLHSEVQ CLSVLEPQGA EGEDTKHFDL |
| 251 | MVGCYGKDSY LREGKILQQL LGTSLGTVPW VNVMHTLPSR YRSRLSLPIN |
| 301 | TEKDKTELYK EISRTHHQLH TLGMGLGAQD SGLLLDRQRL HAPLSQGSHC |
| 351 | HSYLADLTHE ELKILLFSAF VDAKNISKKE LREVSLNFAN DTSVECGCAF |
| 401 | YF* |

The cp6739 nucleotide sequence <SEQ ID 248> is:

| | |
|---|---|
| 1 | ATGACTCATT GCTTACATGG TTGGTTTTCT GTAGTTCGTC ATCACTTTGT |
| 51 | GCAGGCGTTT AATTTCTCAC GTCCTTTATA TTCTCGAATT ACCCACTTCG |
| 101 | CTTTAGGGGT GATTAAGGCC ATCCCCATTG TAGGGCATCT TGTTATGGGA |
| 151 | GTCGATTGGT TGATCTCTCA TTGCTTCGAG AGGGGAGTCT CACACCCTGG |
| 201 | GTTCCCTTCA GATATTGCTC CTATACTGAA AGTAGAAAAG ATCGCGGGCC |
| 251 | GAGATCATAT TTCTAGAATC GAAAATCAGC TAAAGAGCCT TAGGAAAACT |
| 301 | ATCGAGGTTG AAGATCTAGA TAAAGTCCAC GGGCAATATC AAGAGAATCC |
| 351 | TTATGCAGAT ATGGCCTCTA GTGAGGTTCT TAAACTCGAT AAGGGAGTTC |
| 401 | ATGTTAGCGA GCTTGGCAAA GCCTTTTCTA GAGTTCGCAA TCGCATCACC |
| 451 | AGATCCTATA GTTATGCCCC TACTCCTCAG TTGGACTCTA TAGCTATTGT |
| 501 | TGGTATAGAT CTCGTCAGTC CTGAAQAACA AGAGAATTTA GTACGCTTGG |
| 551 | CGAATGAGGT CATTCAACTC TATCCCAAAT CAAAGACAAC TCTATATCTT |
| 601 | CTTATCGATT TTAATAAGGA GTGGGTAGGG GATATCTCCT CTGATAAGGA |
| 651 | AAAACAGCTC CGTTCTCTAG GTCTACATTC TGAAGTTCAG TGTCTTTCCG |
| 701 | TCTTGGAACC TCAGGGTGCC GAGGGCGAAG ATACGAAACA CTTTGACCTT |
| 751 | ATGGTCGGCT GTTATGGGAA GGATTCTTAC TTAAGGGAGG GTAAAATTTT |
| 801 | ACAGCAGGCC CTAGGGACTT CGTTAGGTAC TGTTCCCTGG GTGAATGTTA |
| 851 | TGCACACATT GCCATCTAGG TATAGATCTC GGCTTTCCTT ACCTATAAAT |
| 901 | ACCGAAAAGG ATAAGACAGA GCTTTATAAA GAGATTTCTC GTACACACCA |
| 951 | TCAGTTGCAT ACTTTGGGAA TGGGACTTGG AGCCCAGGAT TCAGGATTGC |
| 1001 | TCTTAGACCG GCAACGACTC CATGCTCCTT TATCTCAAGG GTCTCACTGC |
| 1051 | CATTCCTATC TTGCAGATCT CACCCATGAA GAGCTGAAAA TTTTGTTATT |
| 1101 | TTCAGCATTT GTGGATGCTA AGAACATAAG TAAGAAAGAG CTTCGTGAGG |
| 1151 | TATCTCTAAA TTTTGCTAAC GATACTTCCG TAGAGTGTGG CTGCGCTTTT |
| 1201 | TACTTTTAG |

The PSORT algorithm predicts inner membrane (0.2190).

The protein was expressed in E. coli and puifled as a GST-fusion product (FIG. 124A). The recombinant protein was used to immunise mice, whose sera were used in a Western blot (FIG. 124B) and for FACS analysis.

These experiments show that cp6739 is a surface-exposed and immunoaccessible protein, and that it is a useful immunogen. These properties are not evident from the sequence alone.

### Example 125

The following C. pneumoniae protein (PID 4376741) was expressed <SEQ ID249; cp6741>:

| | |
|---|---|
| 1 | MASCLSAWFS IVREHFYRAF DFSLPFCARI TEFVLGVIKG |
| | IPVVGHIIVG |
| 51 | IEWLVSRYLE SFVTKPTFVS DVVSLLKTEK VAGRDHIARV |
| | VETLKRQRVA |
| 101 | VAPEDEDKVH GKIPVHPFGG IQPVEVLTLY PEVQDATLGL |
| | AFSKIRNRVR |
| 151 | QAYLQAPRPK LQKIYIIGND MNPFEVDDFL HLARLCNETQ |
| | RLYPDATISL |
| 201 | YLTASGGRNA MDKKNRRLLS DCELNPKIAC LDFNQGDVVK |
| | QATCDCWMVY |
| 251 | HGENDQGTLN QIQEELEKSG EETPWIHVGQ KPLSQBLNDF |
| | SPFSSLEMKG |
| 301 | DKEKALEYSE LEKEQLYSRL VYVGERSSVL SLGFGDSRSG |
| | ILMDPKRVHA |
| 351 | PLSEGHYCHS YLADLEWPGL QKTILAAPLN PKELSSTILQ |
| | PISLNLILNS |
| 401 | KTYLRQHPGF FERNSRSDRN VVVVVCDSWW GTDWKEEPSF |
| | QHFIMELECR |
| 451 | GYSHFNIFAF RSNSMCVEER RILNESSQEK AFTMIFCEDS |
| | VSQGDIRCLH |
| 501 | LASEGMLCGK ECYAVDVYTS GCANFMMEEV LTLERESNLW |
| | NRKHGLWKRE |
| 551 | VRKQKQEAAL DQDESEIYVC NQLTAQQNPA CS* |

The cp6741 nucleotide sequence <SEQ ID 250> is:

| | |
|---|---|
| 1 | ATGGCTTCTT GTTTATCTGC CTGGTTTTCT ATAGTTCGTG |
| | AGCACTTTTA |
| 51 | TCGAGCCTTT GATTTTTTCT TGCCGTTTTG TGCTCGTATT |
| | ACGGAATTTG |
| 101 | TATTAGGGGT CATCAAGGGG ATCCCTGTTG TGGGTCACAT |
| | TATTGTTGGG |
| 151 | ATAGAGTGGC TCGTTTCTAG GTATTTAGAG AGTTTCGTGA |
| | CCAAGCCGAC |
| 201 | ATTTGTCTCT GATGTGGTGA GTCTTCTGAA AACAGAGAAA |
| | GTTGCTGGTC |
| 251 | GCGATCACAT TGCTCGTGTh GTGGAGACTT TGAAGAGGCA |
| | GAGAGTCGCT |
| 301 | GTGGCTCCTG AAGATGAGGA TAAGGTCCAT GGGAAGATTC |
| | CTGTGCATCC |
| 351 | TTTCGGGGGA ATCCAACCTG TAGAAGTTCT CACTCTCTAT |
| | CCCGAAGTTC |
| 401 | AAGATGCAAC GTTAGGGCTT GCCTTCTCTA AAATTCGTAA |
| | TCGTGTAAGA |
| 451 | CAGGCGTATT TGCAAGCTCC ACGQCCAAAA CTGCAGAAGA |
| | TTTACATCAT |
| 501 | AGGAAACGAT ATGAATCCTT TTGAAGTTGA CGACTTCTTG |
| | CATCTAGCCC |
| 551 | GTCTCTGTAA TGAAACTCAA AGACTCTATC CTGACGCTAC |
| | GATTTCTCTA |
| 601 | TATCTAACAG CTTCTGGTGG TCGCAATGCT ATGGACAAAA |
| | AGAATCGGAA |
| 651 | GTTACTTAGT GATTGCGAAC TAAACCCCAA GATTGCTTGT |
| | TTGGACTTTA |
| 701 | ATCAGGGTGA TGTAGTCAAA CAAGCAACTT GTGACTGTTG |
| | GATGGTGTAT |
| 751 | CATGGGCAGA ATGATCAAGG TACGTTGAAT CAGATTCAGG |
| | AAGAGTTAGA |
| 801 | AAAGTCAGGG GAGGAAACCC CTTGGATTCA TGTGGGGCAA |
| | AAGCCTCTTT |
| 851 | CACAATCCTT GTGGGATTTC TCTCCATTTT CATCTTTGGA |
| | GATGAAGGGA |
| 901 | GATAAAGAGA AAGCTCTAGA GTACTCTGAA TTAGAAAAAG |
| | AACAGCTATA |
| 951 | TTCTCGATTG GTATACGTAG GAGAGCGCTC TTCGGTTCTT |
| | AGTTTGGGGT |
| 1001 | TTGGAGATAG TCGGGCAGGG ATCTTGATGG ACCCAAAACG |
| | GGTGCATGCT |
| 1051 | CCCTTATCTG AAGGGCATTA TTGTCATTCC TACCTTGCAG |
| | ACTTAGAAAA |
| 1101 | TCCCGGGTTA CAAAAAACAA TTTTAGCGGC ATTTCTGAAT |
| | CCTAAGGAGT |
| 1151 | TGAGCAGTAC CATACTGCAA CCTATATCTC TAAATCTTAT |
| | CTTAAATAGC |
| 1201 | AAAACTTACT TAAGGCAGCA CTTIGGCTTT TTTGAGAGGA |
| | TGAGCAGAAG |
| 1251 | TGATCGCAAT GTGGTTGTCG TTGTATGTGA TTCTTGGTGG |
| | GGTACCGACT |
| 1301 | GGAAGGAGGA GCCAAGCTTC CAACACTTTA TTATGGAGCT |
| | AGAGTGTCGA |
| 1351 | GGGTATTCGC ACTTCAATAT TTTTGCCTTT AGATCTAATA |
| | GCATGTGTGT |
| 1401 | AGAAGAACGT AGGATCTTAA ATGAAAGTTC TCAAGAGAAA |
| | GCCTTTACCA |
| 1451 | TGATTTTCTG TGAGGATTCA GTATCTCAAG GAGATATCCG |
| | CTGTTTGCAT |
| 1501 | TTGGCGTCTG AAGGAATGCT TTGTGGTAAA GAGTGCTATG |
| | CTGTCGATGT |
| 1551 | CTATACCTCA GGATGCCCGA ACTTTATGAT GGAAGAAGTC |
| | TTAACTTTGG |
| 1601 | AGCGAGAATC TAATCTGTGG AATAGAAAGC ATGGTCTTTG |
| | GAAAAGAGAA |
| 1651 | GTTAGAAAAC AGAAACAAGA AGCTGCTTTG GATCAAGACG |
| | AGAGCGAGAT |
| 1701 | TTACGTTTGT AATCAGCTGA CGGCGCAACA GAACTTCGCT |
| | TGTTCTTGA |

The PSORT algorithm predicts inner membrane (0.2869).

The protein was expressed in E. coli and purified as a GST-fusion product (FIG. 125A). The recombinant protein was used to immunise mice, whose sera were used in a Western blot (FIG. 125B) and for FACS analysis.

These experiments show that cp6741 is a surface-exposed and immunoaccessible protein, and that it is a useful immunogen. These properties are not evident from the sequence alone.

### Example 126

The following C. pneumoniae protein (PID 4376742) was expressed <SEQ ID 251; cp6742>:

| | |
|---|---|
| 1 | LFVSNFIFFV VMPIPYISSW ISTVRQHFVK AFDFSRPFCS |
| | RVTNFALGVI |
| 51 | KAIPIVGHIV MGGEWLVSSC VAGIITRSSF TSDVVQIVKT |
| | EKALGRDHIS |
| 101 | RVAEILQRER GTITPENQDK VHGKFPVCPF GRLKSEETLK |
| | LKPGEREGTL |
| 151 | DTVPSPIRTR VTRAYLQAPR PEIRTISIVG SKLKTPQDFS |
| | QFVSLANETQ |
| 201 | RLHPEALVCL YLTGLNRESQ MCDTTTAEKK QYLHNSGLDS |
| | RIQCKDSKBD |
| 251 | DAGSPENPEL WZGYYSREQQ HNIDGQYIQQ CLGKSADPIP |
| | WIHVTEDTKD |
| 301 | FYYPPNFTSY SHRTQSTDPT SPPRLPESEG DKDSLYGQLS |
| | RSYHHEYMLG |
| 351 | LGLKPEDAGL LMDPDRIYAP LSQGHYCHSY LADIENEDLR |
| | TLVLSPFLDP |
| 401 | GNLSSEDLRP VAFNIARLPL ELDSLEFRLV AGQQEGRNTV |
| | TLAHGTPRPE |
| 451 | DLDPDSMNIL TRRLQMSGYS YLNIFSYKSR KMIVKERQFF |
| | GDRSEGKSFT |
| 501 | LILPBDPISA ADFRCLQLAA EGMVARDLPS VADICASGCS |
| | CIQFSEMQSP |
| 551 | QAIEYRQWEA RVEDEAGEEA REPVIYSQDQ LSSHLTTQQN |
| | FVFSLDAVVK |
| 601 | QAIWRFRSKG LLTMERKALG EEFLTAIFSY LGSQERNENM |
| | GXRTTEEHBV |
| 651 | VISFEELDHM VQVLPAEVPA DSGNDPTRPV PNPDSNPDSS |
| | QNEGS* |

The cp6742 nucleotide sequence <SEQ ID 252> is:

| | |
|---|---|
| 1 | TTGTTTGTPT CTAATTTTAT TTTTTTTGTT GTTATGCCAA |
| | TTCCCTATAT |
| 51 | TTCTTCTTGG ATTTCTACCG TTCGACAGCA TTTTGTTAAG |
| | GCCTTTGATT |
| 101 | TCTCTCGTCC CTTTTGTTCT AGGGTTACGA ATTTTGCTTT |
| | AGGGGTCATC |
| 151 | AAGGCCATCC CTATTGTAGG ACATATTGTC ATGGGGATGG |
| | AGTGGTTAGT |
| 201 | TTCTTCCTGT GTTGCCGGGA TTATTACTAG GTCCTCCTTT |
| | ACCTCAGATC |
| 251 | TCGTTCAGAT TGTAAAGACT GAGAAGGCGT TAGGTCGAGA |
| | TCATATATCT |
| 301 | CGAGTGGCGG AGATATTGCA AAGAGAAAGG GGGACCATAA |
| | CTCCTGAGAA |
| 351 | TCAAGATAAG GTGCATGGGA AGTTTCCTGT CTGTCCTTTT |
| | GGTCGTTTAA |
| 401 | AATCCGAGGA AACTTTAAAA CTTAAGCCGG GACAAAGAGA |
| | GGGAACTTTA |
| 451 | GATACTGTAT TTTCTCCGAT TCGCACGCGC GTGACTCGTG |
| | CGTACTTACA |
| 501 | GGCCCCCCGA CCCGAAATAC GTACGATTTC TATTGTGGGT |
| | TCGAAACTTA |
| 551 | AAACTCCTCA AGATTTCTCG CAATTTGTGA GTCTCGCGAA |
| | TGAAACGCAG |
| 601 | AGACTGCATC CTGAAGCGTT AGTTTGTCTG AATTTGACAG |
| | GCTTGAATCG |
| 651 | CGAATCTCAG ATGTGCGATA CACCTACTGC AGAGAAGAAG |
| | CAGTACCTAC |
| 701 | ATAACTCAGG TCTCGACTCT AGAATCCAGT GCAAAGACAG |
| | TAAAGAAGAC |
| 751 | GACGCTGGCT CTCCTGAAAA TCCCGAACTT TGGATTGGCT |
| | ATTATTCACG |
| 801 | AGAGCAACAG CATAATATAG ACGGGCAGTA TATTCAGCAG |
| | TGTCTAGGGA |
| 851 | AGAGTGCAGA TCCAATTCCT TGGATTCATG TTACTGAAGA |
| | CACAAAGGAT |
| 901 | TTTTATTACC CACCAAACTT TACTTCATAC TCACATACAA |
| | GACAATCTAC |
| 951 | AGACCCAACA TCGCCACCAA GACTCCCTGA AAGTGAGGGG |
| | GATAAGGATT |
| 1001 | CCTTGTACGG ACAACTGAGT CGATCGTATC ACCATGAGTA |
| | TATGCTTGGT |
| 1051 | TTGGGATTAA AACCAGAGGA TGCAGGACTC CTGATGGACC |
| | CGGATAGAAT |
| 1101 | CTATGCTCCT CTATCCCAAG GGCATTATTG TCATTCCTAC |
| | CTTGCGGATA |
| 1151 | TAGAAAATGA GGATCTACGA ACTTTAGTCC TTTCGCCTTT |
| | CCTAGATCCT |
| 1201 | GGCAATCTTA GTAGCGAGGA TCTTCGTCCT GTAGCATTCA |
| | ATATCGCTAG |
| 1251 | ATTGCCATTA GAATGGGACT CGTTATTTTT CCGCCTTGTT |
| | GCGGGTCAGC |
| 1301 | AAGAAGGGAG AAACATAGTT ACCCTTGCCC ACGGAACTCC |
| | TCGTCCAGAA |
| 1351 | GATCTTGATC CTGACTCAAT GAACATTCTG ACCAGAAGAT |
| | TACAAATGTC |
| 1401 | TGGATATAGC TATTTGAACA TTTTCTCCTA TAAATCACGG |
| | AAAATGATTG |
| 1451 | TAAAAGAACG TCAGTTCTTT GGAGATCGTT CTGAAGGGAA |
| | GTCTTTCACA |
| 1501 | TTGATCTTAT TTGAGGATCC CATTAGTGCA GCAGATTTCC |
| | GTTGTTTGCA |
| 1551 | GCPAGCTCCA CAAGGTATGG TTCCTAAGGA TCTCCCCAGC |
| | GTAGCAGATA |
| 1601 | TTTGTGCCTC TGGATGTTCC TGCATTCAGT TTTCTGAGAT |
| | GCAGAGTCCT |
| 1651 | CAGGCTATTG AATATAGACA ATGGGAGGCA CGTGTCGAAG |
| | ATGAAGCAGG |
| 1701 | AGAAGAAGCC AGAGAACCAG TAATTTATTC TCAGGATCAA |
| | TTGAGCAGCA |
| 1751 | TGCTCACTAC ACAACAGAAT TTTGTATTTT CTCTAGATGC |
| | TGTGGTAAAA |
| 1801 | CAGGCGATCT GGAGATTCCG TTCGAAAGGT CTTCTTACTA |
| | TGGAAAGAAA |
| 1851 | GCCACTAGGC GAGGAGTTCT TAACTGCGAT ATTTTCCTAT |
| | TTAGGGAGTC |
| 1901 | AGGAGCGTAA TGAGAATATG GGGAAAAGAA CTACCGAAGA |
| | ACATGAGGTC |
| 1951 | GTTATCAGCT TCGAAGAGCT AGATCGCATG GTGCAAGTCC |
| | TCCCAGCCGA |
| 2001 | AGTCCCTGCA GATTCAGGCA ATGATCCTAC GCGTCCCGTT |
| | CCTAATCCAC |
| 2051 | ATAGTAACCC TGATTCCTCG CAAAATGAAG GCAGTTAG |

The PSORT algorithm predicts inner membrane (0.2338).

The protein was expressed in E. coli and purified as a GST-fusion product (FIG. 126A). The recombinant protein was used to immunise mice, whose sera were used in a Western blot (FIG. 126B) and for FACS analysis.

These experiments show that cp6742 is a surface-exposed and immunoaccessible protein, and that it is a useful immunogen. These properties are not evident from the sequence alone.

### Example 127

The following C. pneumoniae protein (PID 4376744) was expressed <SEQ ID 253; cp6744>:

| | |
|---|---|
| 1 | VIQHLLNFAL EETPSISVQY QEQEKLSPCD HSPEIGKKKR |
| | WNKLESFSTY |
| 51 | CSLFMSVKDH YKLNLGIQNS LSGWLLDPYR VCAPLSSPYS |
| | CPSYLLDLQN |
| 101 | KELRRSLLST FLDPKNLTSE TFRSVSINFG NSSPGQRWSE |
| | FLSRVLHDEK |
| 151 | EKHVAVVCND AKLLEEGLSP EALSLLEEDL RESGYSYLNI |
| | LSVSPEGVSK |
| 201 | VQERQILRRD LQGRSFTVMI TDLPLGSRDI RSLQLASDRX |
| | LVSSSLDAAD |
| 251 | ACASGCKVLV YENPNASWAQ ELENPYKQVE RRR* |

The cp6744 nucleotide sequence <SEQ ID 254> is:

| | |
|---|---|
| 1 | GTGATACAAC ATCTTCTAAA CTTTGCTCTA GAAGAGACCC |
| | CTTCCATTTC |
| 51 | CGTGCAATAC CAAGAACAAG AGAAGCTCTC TCCGTGCGAT |
| | CATTCCCCAG |
| 101 | AAATAGGTAA AAAGAAAAGA TGGAATAAGC TGGAATCCTT |
| | CTCCACGTAT |
| 151 | TGTTCTCTGT TTATGTCTGT TAAGGATCAT TATAAGCTGA |
| | ATCTAGGAAT |
| 201 | TCAGAATTCC CTGTCAGGGT GGCTTCTGGA TCCCTATAGG |
| | GTTTGCGCGC |
| 251 | CTTTATCTTC ACCGTACTCG TGTCCTTCCT ATCTTTTAGA |
| | TTTGCAAAAC |
| 301 | AAAGAGCTAC GTCGTTCCCT TCTGTCAACG TTTCTAGACC |
| | CTAAAAATCT |
| 351 | CACTAGCGAA ACATTCCGTT CTGTCTCTAT AAACTTTGGC |
| | AACTCTTCGT |
| 401 | TTGGACAGAG ATGGTCAGAG TTTCTATCTC GTGTTCTGCA |
| | CGACGAGAAA |
| 451 | GAAAAGCACG TAGCTGTTGT TTGTAATGAT GCAAAACTTC |
| | TGGAAGAAGG |
| 501 | ATTGTCCCCA GAGGCATTGT CTCTATTAGA AGAAGACTTA |
| | AGAGAATCAG |
| 551 | GGTATTCGTA TCTAAACATT CTCTCGGTGA GCCCCGAAGG |
| | AGTCTCCAAG |
| 601 | GTTCAGGAAC GTCAGATTCT AAGGCGAGAT CTCCAAGGAC |
| | GGTCCTTTAC |
| 651 | TGTCATGATT ACAGATCTTC CTTTAGGTAG CGAAGATATC |
| | CGTAGTTTAC |
| 701 | AATTAGCCTC GGATAGGATT TTAGTCTCCA GTTCTCTTGA |
| | TGCCGCGGAT |
| 751 | GCATGTGCTT CGGGATGTAA AGTCTTAGTC TACGAAAATC |
| | CAAATGCATC |
| 801 | CTGGGCTCAG GAATTGGAGA ACTTCTACAA ACAAGTTGAG |
| | AGAAGAAGGT |
| 851 | AG |

The PSORT algorithm predicts cytoplasm (0.3833).

The protein was expressed in E. coli and purified as a GST-fusion product (FIG. 127A). The recombinant protein was used to immunise mice, whose sera were used in a Western blot (FIG. 127B) and for FACS analysis.

These experiments show that cp6744 is a surface-exposed and immunoaccessible protein, and that it is a useful immunogen. These properties are not evident from the sequence alone.

### Example 128

The following C. pneumoniae protein (PID 4376745) was expressed <SEQ ID 255; cp6745>:

| | |
|---|---|
| 1 | VACPSISSWF TVVRQHFVNA FDFTHPVCSR ITNFALGIIK |
| | AIPVLGHIVM |
| 51 | GIEWLISWIP RHTVRHGMFT SDVSSAIKVE QTRGHNCLAP |
| | LEAYLSSLRV |
| 101 | PISQEDLGKV HGRTPEDPFV DITPTEIVQL LPDEELSTVD |
| | EALQGVRSRL |
| 151 | TYAYRSVEKP MIQDLALVCF GLRDSADLIN FVRLANGVQN |
| | HYPHTKVKLY |
| 201 | LAKNLADVWD CEISEEEKGQ LRALGLDPKI ESISLTSAGL |
| | PSVPEVATVD |
| 251 | FMITCYGKDQ EVQDP* |

The cp6745 nucleotide sequence <SEQ ID 256> is:

| | |
|---|---|
| 1 | GTGGCTTGTC CAAGTATTTC TTCTTGGTTT ACTGTCGTTC |
| | GACAGCATTT |
| 51 | TGTAAACGCC TTTGATTTCA CCCATCCCGT TTGTTCTCGG |
| | ATTACAAATT |
| 101 | TTGCTTTGGG GATCATTAAG GCAATTCCCG TATTAGGACA |
| | CATTGTCATG |
| 151 | GGAATCGAGT GGTTGATTTC CTGGATTCCC AGACACACCG |
| | TTCGTCATGG |
| 201 | AATGTTTACT TCTGATGTCT CTAGTGCTAT TAAAGTAGAA |
| | CAAACACGGG |
| 251 | GTCATAATTG TTTAGCTCCC CTAGAAGCCT ATTTAAGTAG |
| | CTTGAGAGTC |
| 301 | CCCATTTCCC AAGAAGATCT AGGCAAAGTA CACGGGAGAA |
| | CCCCAGAAGA |
| 351 | TCCCTTCGTA GATATCACAC CCACAGAAAT TGTCCAACTT |
| | CTCCCTGATG |
| 401 | AAGAACTCTC TACTGTAGAT GAGGCACTGC AAGGCGTTCG |
| | TAGTAGGTTA |
| 451 | ACCTATGCCT ATAGGTCCGT AGAGAAACCT ATGATTCAAG |
| | ATCTTGCTCT |
| 501 | TGTGGGTTTT GGTCTCCGAG ATTCTGCGGA CCTCATAAAT |
| | TTCGTGCGTC |
| 551 | TTGCTAATGG CGTGCAGAAT CACTATCCCC ATACTAAAGT |
| | GAAGCTCTAT |
| 601 | TTAGCGAAGA ACTTGGCAGA TGTCTGGGAC TGTGAAATTT |
| | CTGAAGAGGA |
| 651 | AAAAGGGCAA CTCCGAGCTC TAGGTTTAGA CCCTAAAATA |
| | GAGAGTATAT |
| 701 | CCCTTACGAG TGCAGGTCTT CCTTCAGTGC CAGAAGTCGC |
| | TACTGTCGAT |
| 751 | TTTATGATTA CCTGTTACGG GAAAGATCAG GAAGTCCAAG |
| | ATCCCTAG |

The PSORT algorithm predicts inner membrane (0.2253).

The protein was expressed in E. coli and purified as a GST-fusion product (FIG. 128A). The recombinant protein was used to immunise mice, whose sera were used in a Western blot (FIG. 128B) and for FACS analysis.

These experiments show that cp6745 is a surface-exposed and immunoaccessible protein, and that it is a useful immunogen. These properties are not evident from the sequence alone.

### Example 129

The following C. pneumoniae protein (PID 4376747) was expressed <SEQ ID 257; cp6747>:

| | |
|---|---|
| 1 | MMKQGVGQDA KELYTFLSRG NEHYQPCLWF SLEEELGFLF |
| | DEKMLCAPLS |
| 51 | EDHYCHSYLV DLVDQHLKDL ILSMFLDPQN ISAGELLKVS |
| | INVGDSFSPL |
| 101 | QQKDFLSMVL RDETGKNVVV VFKGVLSLPA TQVCKLVEEL |
| | NSKDYSYLNI |
| 151 | FSCHGDSSPQ LLFRKELEGT SGRYFTVICA LYLGDTDMRS |
| | LQLASERIMV |
| 201 | SREFDLVDAY AARCKLLKID HTNWRPGTFS RHADFADAVD |
| | VBAGPNSREF |
| 251 | KLITQANQGI LESGELPLPS KTFWEGFLAF CDRVTVTRHF |
| | IPMLDAAIKQ |
| 301 | AVWTHKHPSL IDKECEALDL RTQCLPSIVS YLEYVTNSHB |
| | KTSKGPFIQK |
| 351 | EIIADCSPLK EALFPGSDED VPSTSEDPSD DHPSDLEDS* |

The cp6747 nucleotide sequence <SEQ ID 258> is:

| | |
|---|---|
| 1 | ATGATGAAAC AAGGAGTCGG GCAGGATGCT AAAGAGCTAT |
| | ACACATTTCT |
| 51 | ATCTCGTGGG AATGAGCATT ACCAACCGTG TCTATGGTTC |
| | AGTCTCGAAG |
| 101 | AGGAACTCGG ATTCCTTTTC GATGAAAAAA TGCTCTGCGC |
| | CCCTCTATCT |
| 151 | GASGATCACT ATTGCCAGTC GTATCTTGTA GATCTAGTGG |
| | ATCAACATTT |
| 201 | AAAGGATTTA ATATTATOGA TGTTTTTAGA TCCTCAGAAT |
| | ATCTCAGCAG |
| 251 | GAGAACTCCT CAAGGTCTCT ATAAACGTTG GAGATTCTTT |
| | TTCTCCTCTA |
| 301 | CAACAGAAAG ATTTCCTCTC GATGGTCTTA CGTGATGAAA |
| | CGGGAAAAAA |
| 351 | CGTCGTCGTG GTTTTTAAAG GAGTTCTCTC CTTACCCGCA |
| | ACCCAAGTCT |
| 401 | GCAAATTAGT AGAGGAATTG AACTCTAAGG ACTACTCCTA |
| | CCTCAATATA |
| 451 | TTTTCTTGTC ACCGAGATAG TAGTCCTCAG CTTTTATTCC |
| | GTAAGGAATT |
| 501 | AGAGGGAACT TCAGGGCGTT ATTTTACAGT GATTTGCGCT |
| | TTATATCTAG |
| 551 | GGGATACAGA CATGCGTAGT TTACAACTTG CTTCTGAAAG |
| | GATCATGGTC |
| 601 | TCTAGAGAGT TTGATCTTGT AGATGCCTAT GCTGCAAGAT |
| | GCAAGCTCTT |
| 651 | GAAAATCGAT CATACAAATT GGAGACCTGG AACTTTCAGT |
| | CGCCACGCCG |
| 701 | ATTTCGCAGA TGCTGTAGAC GTATCAGCAG GATTTAACTC |
| | AAGAGAATTT |
| 751 | AAACTGATTA CGCAGGCGAA TCAAGGGATC CTAGAGTCTG |
| | GAGAACTCCC |
| 801 | GCTCCCTTCA AAAACCTTCT GGGAAGGATT CTTAGCATTC |
| | TGTGATCGAG |
| 851 | TGACTGTCAC GAGACACTTC ATTCCAATGT TAGACGCCGC |
| | TATAAAGCAA |
| 901 | GCGGTATGGA CTCATAAACA TCCCAGCTTG ATAGATAAAG |
| | AGTGTGAAGC |
| 951 | CCTAGACTTG AAAACACAGT GCTTGCCATC TATCGTATCG |
| | TACCTTGAAT |
| 1001 | ATGTCACAAA CTCTCACGAA AAAACATCGA AAGGCCCGTT |
| | CATACAAAAA |
| 1051 | GAGATTATCG CAGACTGTTC TCCTCTTAAA GAGGCGCTCT |
| | TCCCAGGTTC |
| 1101 | TGATGAAGAT GTTCCCTCTA CCTCTGAGGA TCCTTCAGAT |
| | GATCATCCTT |
| 1151 | CGGATCTTGA AGACTCTTAA |

The PSORT algorithm predicts inner membrane (0.1447).

The protein was expressed in E. coli and purified as a GST-fusion product (FIG. 129A) and also as a his-tagged product. The recombinant proteins were used to immunise mice, whose sera were used in a Western blot (FIG. 129B) and for FACS analysis.

These experiments show that cp6747 is a surface-exposed and immunoaccessible protein, and that it is a useful immunogen. These properties are not evident from the sequence alone.

### Example 130

The following C. pneumoniae protein (PID 4376756) was expressed <SEQ ID 259; cp6756>:

| | |
|---|---|
| 1 | NASGIGGSSG LGKIPPKPNG DRSRSPSPKG ELGSHEISLP |
| | PQEHGEEGAS |
| 51 | GSSHIHSSSS FLPBDQEBQS SSSAASSPGF FSRVRSGVDR |
| | ALKSFGNFFS |
| 101 | AESTSQARET RQAFVRLSRT ITADERRDVD SSSAAATEAR |
| | VAEDASVSGE |
| 151 | NPSQGVPETS SGPEPQRLFS LPSVKKQSGL GRLVQTVRDR |
| | IVLPSGAPPT |
| 201 | DSEPLSLYEL NLRLSSLRQE LSDIQSNDQL TPEEKAEATV |
| | TIQOLIQITE |
| 251 | FQCGYMEATQ SSVSLAEARF KGVETSDEIN SLCSELTDPE |
| | LQELMSDGDS |
| 301 | LQNLLDETAD DLEAALSHTR LSFSLDDNPT PIDNNPTLIS |
| | QEEPIYEEIG |
| 351 | GAADPQRTRE NWSTRLWNQI REALVSLLGM ILSILGSILH |
| | ELRIARHAAA |
| 401 | EAVGRCCTCR GEECTSSEED SNSVGSPSEI DETERTGSPH |
| | DVPRRNGSPR |
| 451 | EDSPLMNALV GWAHKHGAKT KESSESSTPE ISISAPIVRG |
| | WSQDSSVSFI |
| 501 | VNEDDHIFYD VPRRKDGIYD VPSSPRWSPA EELEEDVFGD |
| | YEVPITSAEP |
| 551 | SKDKNIYMTP RLATPAIYDL PSRPGSSGSS RSPSSDRVRS |
| | SSPNRRGVPL |
| 601 | PPVPSPAMSE EGSIYEDMSG ASGAGESDYE DMSRSPSPRG |
| | DLDEPIYANT |
| 651 | PEDNPFTQRN IDRILQERSG GASASPVEPI YDEIPWIHGR |
| | PPATLPRPEN |
| 701 | TLTNVSLRVS PGFGPEVRAA LLSESVSAVM VEAESIVPPT |
| | EPGDGESEYL |
| 751 | EPLGGLVATT KILLQKGWPR GESNA* |

The cp6756 nucleotide sequence <SEQ ID 260> is:

| | |
|---|---|
| 1 | ATGGCATCAG GAATCGGAGG ATCTAGTGGA TTAGGAAAGA |
| | TTCCACCTAA |
| 51 | AGATAATGGG GATAGAAGTC GATCGCCCTC TCCTAAGGGA |
| | GAACTTGGCA |
| 101 | GCCACGAGAT TTCCCTGCCT CCTCAAGAAC ATGGAGAGGA |
| | AGGAGCTTCA |
| 151 | GGATCTTCGC ATATACATAG CAGTTCCTCT TTTCTACCAG |
| | AAGATCAGGA |
| 201 | GTCTCAGAGC TCTTCTTCGG CAGCTTCTAG CCCGGGATTT |
| | TTTTCTCGCG |
| 251 | TACGTTCTGG GGTAGACAGG GCCTTAAAAT CATTTGGCAA |
| | CTTTTTTTCC |
| 301 | GCAGAGTCTA CGAGTCAAGC GCGTGAAACG CGACAAGCTT |
| | TTGTTAGATT |
| 351 | ATCAAAAACC ATCACCGCGG ATGAGAGACG GGATGTCGAT |
| | TCATCAAGTG |
| 401 | CTGCTGCTAC AGAAGCCCGA GTGGCAGAGG ACGCGAGTGT |
| | TTCAGGCGAA |
| 451 | AATCCTTCTC AGGGGGTTCC AGAAACCTCT TCTGGACCAG |
| | AACCTCAGCG |
| 501 | TTTATTTTCT CTTCCTTCAG TAAAAAAACA GAGCGGTTTG |
| | GGTCGGTTGG |
| 551 | TACAGACAGT TCGCGATCGC ATACTACTTC CTAGTGGGGC |
| | TCCACCTACA |
| 601 | GACAGCGAGC CTTTAAGTCT CTACGAGCTA AACCTCCGTT |
| | TGAGTAGTTT |
| 651 | ACGTCAGGAG CTCTCTGACA TACAAAGTAA TGATCAGTTG |
| | ACTCCAGAGG |
| 701 | AAAAAGCAGA AGCCACAGTT ACCATACAAC AGCTGATCCA |
| | AATTACAGAA |
| 751 | TTCCAATGCG GCTATATGGA GGCAACACAA TCTTCGGTAT |
| | CTCTAGCAGA |
| 801 | AGCTCGTTTT AAGGGGGTAG AAACTAGTGA TGAGATCAAT |
| | TCCCTCTGTT |
| 851 | CAGAACTGAC AGATCCTGAG CTTCAAGAAC TCATGAGTGA |
| | TGGAGACTCT |
| 901 | CTTCAAAACC TATTAGATGA GACTGCCGAC GATTTAGAAG |
| | CTGCTTTGTC |
| 951 | CCATACTCGA TTGAGTTTTT CTTTAGACGA TAATCCAACT |
| | CCGATAGACA |
| 1001 | ATAATCCAAC TCTGATTTCT CAAGAAGAGC CTATTTATGA |
| | GGAAATCGGA |
| 1051 | GGAGCTGCAG ATCCTCAAAG AACTCGGGAA AACTGGTCTA |
| | CAAGATTATG |
| 1101 | GAATCAGATT CGCGAGGCTC TGGTTTCTCT TTTAGGAATG |
| | ATTTTAAGCA |
| 1151 | TTCTAGGGTC CATCTTGCAC AGGTTGCGTA TTGCTCGTCA |
| | TCCAGCTCCT |
| 1201 | GAAGCAGTGG GTCGTTGTTG CACGTGCCGA GGAGAAGAGT |
| | GTACTTCTTC |
| 1251 | TGAAGAGGAC TCGATGTCGG TGGGGTCTCC TTCAGAAATT |
| | GATGAAACTG |
| 1301 | AAAGAACGGG CTCTCCGCAT GACGTTCCAC GCAGAAATGG |
| | AAGTCCACGT |
| 1351 | GAAGATTCTC CATTGATGAA TGCCTTAGTA GGATGGGCAC |
| | ATAAGCACGG |
| 1401 | TGCTAAAACC AAGGAGAGTT CAGAATCAAG TACCCCGGAA |
| | ATTTCGATTT |
| 1451 | CTGCTCCCAT AGTCAGAGGT TGGAGTCAAG ACAGTTCCGT |
| | CAGTTTTATT |
| 1501 | GTTATGGAAG ATGATCATAT TTTCTATGAT GTTCCTCGTA |
| | GAAAAGATGG |
| 1551 | AATCTATGAC GTTCCTAGTT CCCCTAGATG GAGTCCTGCG |
| | CGAGAGTTGG |
| 1601 | AAGAGGATGT TTTTGGAGAT TATGAAGTTC CTATAACCTC |
| | TGCTGAACCA |
| 1651 | TCTAAAGACA AGAACATCTA CATGACACCT AGATTAGCAA |
| | CTCCTGCTAT |
| 1701 | CTATGATCTT CCTTCACGTC CAGGATCGTC TGGAAGCTCA |
| | CGTTCTCCGT |
| 1751 | CTTCAGATCG CGTACGAAGC AGCTCACCAA ATAGACGGGG |
| | TGTGCCTCTT |
| 1801 | CCTCCAGTTC CTTCACCTGC TATGAGTGAG GAGGGGAGCA |
| | TTTATGAGGA |
| 1851 | TATGAGCGGT GCTTCAGGTG CAGGTGAAAG TGATTATGAA |
| | GATATGAGCC |
| 1901 | GTTCCCCCTC TCCTAGAGGC GACTTGGATG AACCCATATA |
| | TGCTAATACT |
| 1951 | CCTGAAGATA ATCCATTTAC TCAGAGAAAT ATAGATAGAA |
| | TTTTACAGGA |
| 2001 | GAGGTCAGGC GGTGCTTCCG CTTCTCCTGT AGAGCCTATT |
| | TATGATGAGA |
| 2051 | TCCCATGGAT TCATGGCAGG CCCCCTGCTA CACTTCCAAG |
| | ACCCGAGAAT |
| 2101 | ACATTGACTA ATGTTTCGCT TAGAGTGAGC CCAGGGTTTG |
| | GACCAGAAGT |
| 2151 | AAGAGCCGCT TTGCTTAGCG AGAGCGTGAG TGCTGTTATG |
| | GTCGAAGCAG |
| 2201 | AGAGTATTGT TCCTCCAACA GAGCCGGGGG ACGGAGAATC |
| | AGAATATCTA |
| 2251 | GAGCCCTTAG GGGGACTTGT AGCTACAACG AAAATCTTAC |
| | TACAAAAAGG |
| 2301 | ATCCCCTCCT GGAGAGTCGA ATGCTTAG |

The PSORT algorithm predicts inner membrane (0.3994).

The protein was expressed in E. coli and purified as a GST-fusion product (FIG. 130A). The recombinant protein was used to immunise mice, whose sera were used in a Western blot (FIG. 130B) and for FACS analysis.

These experiments show that cp6756 is a surface-exposed and immunoaccessible protein, and that it is a useful immunogen. These properties are not evident from the sequence alone.

### Example 131

The following C. pneumoniae protein (PID 4376761) was expressed <SEQ ID 261; cp6761>:

| | |
|---|---|
| 1 | MTVAEVKGTF KLVCLGCRVN QYEVQAYRDQ LTILGYQEVL |
| | DESEIPADLCI |
| 51 | INTCAVTASA ESSGRHAVRQ LCRQNPTAHI VVTGCLGESD |
| | KEFFASLDRQ |
| 101 | CTLVSNKEKS RLIEKIFSYD TTEPEFKIHS FEGKSRAFIK |
| | VQDGCNSFCS |
| 151 | YCIIPYLRGR SVSRPAEKIL AEIAGVVDQG YREVVIAGIN |
| | VGDYCDGERS |
| 201 | LASLIEQVDR IPGIERIRIS SIDPDDITED LHPAITSSRH |
| | TCPSSHLVLQ |
| 251 | SGSNSILKRM NRKYSRGDFL DCVEKFRASD PRYAFTTDVI |
| | VGFPGESDQD |
| 301 | FEDTLRIIED VGFIKVHSFP FSARRRTKAY TFDNQIPNQV |
| | IYERKKYLAE |
| 351 | VAKRVGQKEM MKRLGETTEV LVEKVTGQVA TGHSPYFEKV |
| | SFPVVGTVAI |
| 401 | NTLVSVRLDR VEEEGLIGEI V* |

The cp6761 nucleotide sequence <SEQ ID 262> is:

| | |
|---|---|
| 1 | ATGACGGTTG CGGAAGTCAA AGGAACATTT AAGCTGGTCT |
| | GTTTAGGCTG |
| 51 | TCGGGTGAAT CAGTATGAGG TCCAAGCATA TCGCGACCAG |
| | TTGACTATCT |
| 101 | TAGGTTACCA AGAGGTCCTG GATTCTGAAA TCCCTGCAGA |
| | TTTATGCATA |
| 151 | ATCAATACGT GTGCTGTCAC AGCTTCTGCT GAGAGTTCGG |
| | GTCGTCATGC |
| 201 | TGTGCGTCAG TTATGTCGTC AGAACCCTAC AGCACATATT |
| | GTTGTCACAG |
| 251 | GTTGTTTGGG GGAATCTGAC AAAGAGTTTT TTGCTTCTTT |
| | GGATCGGCAA |
| 301 | TGCACACTTG TTTCCAATAA AGAAAAATCC CGACTAATAG |
| | AAAAAATTTT |
| 351 | TTCCTATGAT ACGACCTTCC CTGAGTTCAA GATCCATAGT |
| | TTTGAGGGAA |
| 401 | AGTCTCGAGC TTTTATTAAA GTTCAAGATG GCTGTAATTC |
| | TTTTTGCTCG |
| 451 | TACTGCATTA TTCCTTATTT GCGGGGGCGT GCGGTTTCTC |
| | GTCCTGCTGA |
| 501 | GAAGATTTTA GCTGAAATCG CAGGGGTTGT AGACCAAGGA |
| | TATCGCGAAG |
| 551 | TTGTAATTGC AGGAATTAAT GTTGGAGATT ATTGCGATGG |
| | AGAGCGTTCA |
| 601 | TTAGCCTCTT TGATTGAACA GGTGGACCGG ATTCCTGGAA |
| | TTGAGAGGAT |
| 651 | TCGAATTTCC TCTATAGATC CTGATGATAT CACTGAAGAT |
| | CTGCACCGTG |
| 701 | CCATCACCTC ATCGCGTCAC ACTTGTCCTT CGTCACACCT |
| | TGTTCTTCAA |
| 751 | TCGGGGTCGA ATTCAATTTT AAAGAGAATG AACCGGAAGT |
| | ATTCTCGCGG |
| 801 | AGATTTTTTA GATTGTGTAG AGAAGTTCCG TGCTTCTGAT |
| | CCTCGCTATG |
| 851 | CCTTTACTAC AGATGTCATT GTCGGATTTC CTGGAGAGAG |
| | TGATCAAGAT |
| 901 | TTTGAAGATA CTTTGAGAAT TATTGAAGAT GTAGGCTTTA |
| | TTAAAGTGCA |
| 951 | TAGTTTCCCT TTCAGTGCTC GTCGTCGTAC TAAGGCATAT |
| | ACTTTTGATA |
| 1001 | ATCAGATTCC CAATCAGGTG ATCTATGAGA GGAAGAAGTA |
| | TCTTCCTGAG |
| 1051 | GTTGCTAAGA GGGTAGGCCA GAAAGAGATG ATGAAGCGTT |
| | TAGGAGAGAC |
| 1101 | TACAGAGGTG CTTGTTGAGA AAGTAACGGG GCAGGTTGCT |
| | ACGGGTCACT |
| 1151 | CTCCTTATTT TGAAAAGGTT TCTTTCCCTG TTGTAGGAAC |
| | GGTAGCTATC |
| 1201 | AACACTCTAG TTTCTGTGCG TCTTGATAGG GTAGAGGAAG |
| | AAGGGCTGAT |
| 1251 | TGGCGAGATT GTATGA |

The PSORT algorithm predicts inner membrane (0.1574).

The protein was expressed in E. coli and purified as a GST-fusion product (FIG. 131A) and also as a his-tagged product. The recombinant proteins were used to immunise mice, whose sera were used in a Western blot (FIG. 131B) and for FACS analysis.

These experiments show that cp6761 is a surface-exposed and immunoaccessible protein, and that it is a useful immunogen. These properties are not evident from the sequence alone.

### Example 132

The following C. pneumoniae protein (PID 4376766) was expressed <SEQ ID 263; cp6766>:

| | |
|---|---|
| 1 | MATSVPVTSS TSVGEANSSN ERFTERTSRM YYAALVLGAL |
| | SCLIFIAMIV |
| 51 | IFPQVGLWAV VLGFALGCLL LSLAIVPAVS GLVLGKTLEP |
| | SREATPPEIV |
| 101 | AQKEWTTQQD VLGNEYWRSE LISLFKRGDL HESLIVDSKD |
| | RSLDIDOSLO |
| 151 | NILKLEPLST TLSLLKKDCV HINIILHLVR QWNLLGVDLS |
| | PEVTAEAEEL |
| 201 | LLFLIEEQYY SPDILKLIRY GDALQATSPL MDWADSGSFS |
| | VDADGVFSCR |
| 251 | REECSPEDAL AQFDLLLALE NPDRRFLKDS FLTYIWSSSF |
| | FEKFLHRHLE |
| 301 | SLQRKLPETA IDVARYEAQI QTFLSRYFQK LDLINAMSLD |
| | WGYNCAEGEK |
| 351 | CYESANQRLD NLFIAFSSSV PAMKRLFDKY GSVVRVDRRQ |
| | IREQILSNTE |
| 401 | ILENESGPLC SLYEYPLSYL IDWAVLLDCV RGTEISLEDQ |
| | ADYTVCLQGL |
| 451 | DSMLSQFASR LQSGQKVLNP RDVLSEQAAV MLVHGLAAQG |
| | VSFQGLKALM |
| 501 | YLTAVPQRMW LGALPLFESF PVFNRMKEFL GESLGD* |

The cp6766 nucleotide sequence <SEQ ID 264> is:

| | |
|---|---|
| 1 | ATGGCAACCT CTGTTCCTGT AACTTCATCT ACTTCTGTAG |
| | GAGAGGCTAA |
| 51 | CTCCTCCAAC GAAAGATTTA CTGAACGAAC ATCGCGAATG |
| | TATTACGCAG |
| 101 | CTTTAGTCCT AGGGGCTTTG AGCTGTTTAA TTTTTATTGC |
| | TATGATTGTC |
| 151 | ATTTTCCCAC AGGTCGGATT GTGGGCTGTG GTCCTCGGGT |
| | TTGCTCTTGG |
| 201 | ATGTTTACTT TTAAGCTTAG CTATCGTTTT TGCTGTCTCC |
| | GGTCTCGTTT |
| 251 | TAGGCAAGAC TTTAGAACCT AGTCGAGAAG CGACTCCTCC |
| | AGAAATTGTT |
| 301 | GCGCAAAAGG AGTGGACTAC ACAACAAGAT GTCTTAGGGA |
| | ATGAGTATTG |
| 351 | GCGTTCCGAG TTGATTTCCT TGTTCTTACG AGGGGATCTC |
| | CACGAATCTC |
| 401 | TGATTGTTGA TTCTAAGGAT CGATCTTTAG ATATTGATCA |
| | GAGTTTACAA |
| 451 | AATATATTGA AACTTGAGCC CCTATCTACG ACACTTTCGC |
| | TGTTAAAGAA |
| 501 | AGATTGTGTC CACATCAATA TCATTTTACA TTTAGTGAGA |
| | CAGTGGAACT |
| 551 | TACTGGGAGT GGATCTTAGT CCTGAAGTCA CTGCGCACGC |
| | CGAGGAACTT |
| 601 | CTACTCTTTT TGATAGAAGA GCAGTATTAC TCTCCTGATA |
| | TTTTGAAATT |
| 651 | GATTCGCTAC GGAGATGCTT TACAAGCAAC GTCTCCTTTG |
| | ATGGATTGGG |
| 701 | CAGATTCAGG TTCCTTTAGT GTAGACGCAG ACGGGGTATT |
| | TAGCTGTCGC |
| 751 | AGAGAAGAAT GTTCTCCTGA GGATGCTTTG GCGCAATTCG |
| | ATCTTCTTTT |
| 801 | GGCGTTGGAA AATCCCGACA GACGCTTCTT AAAGGATTCT |
| | TTTCTTACCT |
| 851 | ACATTTGGTC GTCTTCATTT TTTGAGAAGT TTTTACATCG |
| | CCATCTAGAG |
| 901 | AGCTTGCAAA GAAAGCTCCC AGAGACAGCG ATCGATGTCG |
| | CCCGCTATGA |
| 951 | AGCACAAATA CAAACATTTC TCTCTCGCTA TTTTCAGAAG |
| | CTCGATTTGA |
| 1001 | TAAACGCAAT GTCCTTAGAT TGGGGATATA ACTGTGCTGA |
| | GGGAGAAAAA |
| 1051 | TGTTATGAGA GCGCAAATCA AAGATTAGAC AACCTATTTA |
| | TTGCTTTTTC |
| 1101 | TTCTTCTGTT CCTGCTATGA AGCGGCTCTT TGACAAATAT |
| | GGTTCTGTGG |
| 1151 | TACGGGTAGA TCGTAGGCAG ATTCGTGAGC AGATTCTTTC |
| | GAACACTGAA |
| 1201 | ATCTTAGAAA ATGAGTCAGG GTTCCTCTGC AGTTTGTATG |
| | AATATCCTTT |
| 1251 | ATCCTATTTG ATAGATTGGG CTGTTTTGCT AGACTGTGTT |
| | CGCGGTACCG |
| 1301 | AAATCTCTCT AGAAGATCAG GCCGATTACA CCGTTTGTTT |
| | GCAAGGCTTG |
| 1351 | GATTCTATGT TATCTCAATT TGCGAGTCGT TTACAGTCTG |
| | GACAAAAAGT |
| 1401 | ATTGAATCCT AGAGATGTTT TAAGTGAACA GGCTGCGGTT |
| | ATGCTTGTTC |
| 1451 | ATGGCTTGGC AGCACAGGGC GTGTCGTTTC AAGGATTGAA |
| | AGCTTTGATG |
| 1501 | TATTTGACAG CCGTTCCCCA AAGAATGTGG TTAGGAGCAT |
| | TGCCTTTATT |
| 1551 | TGAATCTTTT CCTGTCTTTA ATCGGATGAA AGAATTTCTT |
| | GGGGAATCTC |
| 1601 | TGGGAGACTA G |

The PSORT algorithm predicts inner membrane (0.6158).

The protein was expressed in E. coli and purified as a GST-fusion product (FIG. 132A) and also as a his-tagged product. The recombinant proteins were used to immunise mice, whose sera were used in a Western blot (FIG. 132B) and for FACS analysis.

These experiments show that cp6766 is a surface-exposed and immunoaccessible protein, and that it is a useful immunogen. These properties are not evident from the sequence alone.

### Example 133

The following C. pneumoniae protein (PID 4376804) was expressed <SEQ ID 265; cp6804>:

| | |
|---|---|
| 1 | MSNQLQPCIS LGCVSYINSF PLSLQLIKRN DIRCVLAPPA |
| | DLLNLLIEGK |
| 51 | LDVALTSSLG AISHNLGYVP GFGIAANQRI LSVNLYAAPT |
| | FFNSPQPRIA |
| 101 | ATLESRSSIG LLKVLCRHLW TIPTPHILRF ITTKVLRQTP |
| | ENYDGLLLIG |
| 151 | DAALQHPVLP GFVTYDLASG WYDLTKLPFV FALLLHSTSW |
| | KEHPLPNLAM |
| 201 | EEALQQFESS PEEVLKEAHQ HTGLPPSLLQ EYYALCQYRL |
| | GEEHYESFER |
| 251 | FREYYGTLYQ QARL |

The cp6804 nucleotide sequence <SEQ ID 266> is:

| | |
|---|---|
| 1 | ATGTCTAACC AACTCCAGCC ATGTATAAGC TThGGCTGCG |
| | TAAGTTATAT |
| 51 | TAATTCCTTT CCGCTGTCCC TACAACTCAT AAAAAGAAAC |
| | GATATTCGCT |
| 101 | GTGTTCTTGC TCCCCCTGCA GACCTCCTCA ACTTGCTAAT |
| | CGAAGGGAAA |
| 151 | CTCGATGTTG CTTTGACCTC ATCCCTAGGA GCTATCTCTC |
| | ATAACTTGGG |
| 201 | GTATGTCCCC GGCTTTGGAA TTGCAGCAAA CCAACGTATC |
| | CTCAGTGTAA |
| 251 | ACCTCTATGC AGCTCCCACT TTCTTTAACT CACCGCAACC |
| | TCGGATTGCC |
| 301 | GCAACTTTAG AAAGTCGCTC CTCTATAGGA CTCTTAAAAC |
| | TGCTTTGTCG |
| 351 | TCATCTCTGG CGCATCCCAA CTCCTCATAT CCTAAGATTC |
| | ATAACTACAA |
| 401 | AAGTACTCAG ACAAACCCCT GAAAATTATG ATGGCCTCCT |
| | CCTAATCGGA |
| 451 | GATGCAGCGC TACAACATCC TGTACTTCCT GGATTTGTAA |
| | CCTATGACCT |
| 501 | TGCCTCGGGG TGGTATGATC TTACAAAGCT ACCTTTTGTA |
| | TTTGCTCTTC |
| 551 | TTCTACACAG CACCTCTTGG AAAGAACATC CCCTACCCAA |
| | CCTTGCGATG |
| 601 | GAAGAAGCCC TCCAACAGTT CGAATCTTCA CCCGAAGAAG |
| | TCCTTAAAGA |
| 651 | AGCTCATCAA CATACAGGTC TGCCCCCTTC TCTTCTTCAA |
| | GAATACTATG |
| 701 | CCCTATGCCA GTACCGTCTA GGAGAAGAAC ACTACGAAAG |
| | CTTTGAAAAA |
| 751 | TTCCGGGAAT ATTATGGAAC CCTCTACCAA CAAGCCCGAC |
| | TGTAA |

The PSORT algorithm predicts inner membrane (0.060).

The protein was expressed in E. coli and purified as a GST-fusion product (FIG. 133A). The recombinant protein was used to immunise mice, whose sera were used in a Western blot (FIG. 133B) and for FACS analysis.

These experiments show that cp6804 is a surface-exposed and immunoaccessible protein, and that it is a useful immunogen. These properties are not evident from the sequence alone.

### Example 134

The following C. pneumoniae protein (PID 4376805) was expressed <SEQ ID 267; cp6805>:

| | |
|---|---|
| 1 | MSSLLSCGRI RPTRVTCSLK TYLEDTSQNQ LSTRLVRASV |
| | IFLCALLIIL |
| 51 | VCVALSSLIP SIMALATSFT VMGLILFVMS LLGDVAIISY |
| | LTYSTVTSYR |
| 101 | QNKRAFEIHK PARSVYYEGV RHWDLGRSSL GTGEIPIVRT |
| | LFSPFQNHGL |
| 151 | NHALAAKIFL FMEHFSPEPP NEPLVDWACL IRDFRPHVSS |
| | LCFVIEKQGS |
| 201 | SLRTKEGNTI CEAFRSDYDA HFAMVDCYRL IHSKLIIEKM |
| | GLKNIDIIPS |
| 251 | VHVRBPYPSR POEGYREOLL RMYGGKGAL* |

The cp6805 nucleotide sequence <SEQ ID 268> is:

| | |
|---|---|
| 1 | ATGTCATCAC TACTGAGCTG CGGAAGAATA GAQCCGACTC |
| | GGGTTACCTG |
| 51 | TAGCTTAAAG ACGTATCTTG AGGATACGAG TCAGAATCAG |
| | TTGAGCACAC |
| 101 | GTCTAGTTCG GGCAAGTGTC ATCTTTTTAT GCGCATTGTT |
| | GATCATTTTG |
| 151 | GTTTGTGTGG CCCTCTCTAG TTTGATTCCA AGCATTATGG |
| | CCTTGGCGAC |
| 201 | CTCTTTTACG GTAATGGGGT TAATTCTTTT TGTGATGTCA |
| | CTTCTTGCTG |
| 251 | ACGTTGCAAT TATAAGTTAT CTTACTTATA GCACTGTTAC |
| | GAGTTACCGG |
| 301 | CAAAATAAGA GAGCTTTTGA GATTCACAAG CCCGCTCGCT |
| | CCGTTTACTA |
| 351 | CGAGGGGGTC CGCCATTGGG ATTTAGGACG ATCATCTTTA |
| | GGCACAGGCG |
| 401 | AGATTCCTAT AGTAAGGACG TTATTCTCTC CATTTCAGAA |
| | CCATGGTCTT |
| 451 | AACCATGCCT TAGCTGCTAA AATTTTCCTA TTTATGGAGC |
| | ATTTCAGCCC |
| 501 | TGAGCCACCG AACGAGCCTT TGGTGGATTG GGCCTGTTTG |
| | ATTCGGGATT |
| 551 | TTAGGCCTCA CGTCAGTTCT TTGTGCTTTG TTATTGAAAA |
| | ACAAGGGTCA |
| 601 | TCGCTGAGGA CTAAGGAAGG CAATACGATT TGTGAGGCTT |
| | TCCGCTCTGA |
| 651 | TTACGACGCC CATTTTGCTA TGGTAGATTG CTACCGGTTG |
| | ATCCACTCTA |
| 701 | AGTTGATTAT AGAGAAAATG GGATTGAAGA ATATCGATAT |
| | CATTCCGAGT |
| 751 | GTCATGGTTC GTGAAGATTA TCCTAGCCGT CCTGGGGAGG |
| | GCTATCGCGA |
| 801 | AGGCCTATTA CGTATGTATG GTGGCAAGGG GGCTCTGTGA |

The PSORT algorithm predicts inner membrane (0.711).

The protein was expressed in E. coli and purified as a GST-fusion product FIG. 134A). The recombinant protein was used to immunise mice, whose sera were used in a Western blot (FIG. 134B) and for FACS analysis.

These experiments show that cp6805 is a surface-exposed and immunoaccessible protein, and that it is a useful immunogen. These properties are not evident from the sequence alone.

### Example 135

The following C. pneumoniae protein (PID 4376813) was expressed <SEQ ID 269; cp6813>:

| | |
|---|---|
| 1 | MSGPSRTBSS QVSVLSYVPR DKEIAPKKQF TIAKISTLAI |
| | LASLALGALV |
| 51 | AGISLTIVLG NPVFLALLIT TALFSVVTFL VYHQMTSKVS |
| | SNWQKVLEQN |
| 101 | FKPLGKAWQE KNVDCYSNEM QFYNNHLNPK FKVAIQTDAS |
| | QPFQPTFLTG |
| 151 | LRVIEKNQST GIIFNPVGPT NLIDNTATNL STILYSTLKD |
| | KSVWDTCKQR |
| 201 | EGGPAKGEDP FSPTEVRVVK LPNEALDQTF NLNLSSAEKK |
| | SILPTFLGHV |
| 251 | CGPKSEELPN QQEYYRQALL AYENCLKAAI ESHAAIVALP |
| | LFTSVYEVPP |
| 301 | EEILPKEGTF YWDNQTQAFC KRALLDAIQN TALRYPQRSL |
| | LVILQDPFNT |
| 351 | IESQSRSEE* |

The cp6813 nucleotide sequence <SEQ ID 270> is:

| | |
|---|---|
| 1 | ATGTCAGGAC CCTCACGTAC TGAGAGCTCT CAAGTTTCTG |
| | TACTATCCTA |
| 51 | TGTGCCTCGG GATAAAGAAA TTGCTCCTAA AAAACAGTTT |
| | ACCATAGCAA |
| 101 | AAATATCCAC TCTTGCAATC CTAGCTTCTT TAGCTTTAGG |
| | AGCTTTGGTG |
| 151 | GCTGGAATCT CTTTAACGAT AGTATTAGGG AACCCTGTAT |
| | TTTTGGCTCT |
| 201 | TCTCATTACC ACGGCCCTCT TCTCAGTTGT AACCTTCTTA |
| | GTCTACCACC |
| 251 | AAATGACCTC AAAGGTATCT TCTAACTGGC AGAAAGTTCT |
| | AGAGCAAAAC |
| 301 | TTCAAGCCTT TGGGAAAAGC GTGGCAAGAA AAAAACGTAG |
| | ACTGCTACTC |
| 351 | AAACGAGATG CAATTTTACA ATAATCACCT GAACCCTAAG |
| | TTCAAGGTAG |
| 401 | CGATACAAAC AGATGCGTCT CAACCATTTC AGCCTACTTT |
| | CTTAACTGGA |
| 451 | CTTAGAGTGA TCGAAAAAAA TCAATCCACA GGGATCATCT |
| | TTAATCCCGT |
| 501 | AGGCCCAACG AATCTGATCG ACAACACTGC AACGAACCTC |
| | TCTACTATCC |
| 551 | TTTACTCCAC CCTAAAAGAT AAAAGCGTGT GGGATACATG |
| | CAAGCAACGC |
| 601 | GAAGGGGGTC CCGCAAAAGG AGAAGACCCC TTTTCCCCTA |
| | CCGAAGTGAG |
| 651 | AGTAGTAAAA CTTCCAAACG AAGCTCTAGA TCAAACGTTT |
| | AATCTAAATT |
| 701 | TAATCTCTGC AGAAAAGAAA AGTATTCTTC CGACCTTTTT |
| | AGGCCACGTA |
| 751 | TGCOGCCCTA AATCTGAAGA GTTACCAAAT CAGCAAGAAT |
| | ATTATCGCCA |
| 801 | AGCTTTACTA GCGTACGAGA ACTGCCTTAA AGCAGCTATA |
| | GAAAGTCATG |
| 851 | CAGCAATCGT TGCTCTTCCT CTCTTTACTT CGGTCTATGA |
| | AGTGCCTCCA |
| 901 | GAAGAGATTC TTCCTAAAGA AGGCACTTTC TATTGGGACA |
| | ACCAAACTCA |
| 951 | AGCGTTTTGC AAACCCGCTT TATTGGACGC TATTCAAAAT |
| | ACGGCCCTAC |
| 1001 | GCTATCCTCA AAGATCTTTA CTTGTTATAC TCCAAGATCC |
| | TTTTAATACT |
| 1051 | ATAGAATCAC AAAGTCGTTC TGAGGAGTAA |

The PSORT algorithm predicts inner membrane (0.4291).

The protein was expressed in E. coli and purified as a GST-fusion product (FIG. 135A). The recombinant protein was used to immunise mice, whose sera were used in a Western blot (FIG. 135B) and for FACS analysis.

These experiments show that cp6813 is a surface-exposed and immunoaccessible protein, and that it is a useful immunogen. These properties are not evident from the sequence alone.

### Example 136

The following C. pneumoniae protein (PID 4376844) was expressed <SEQ ID 271; cp6844>:

| | |
|---|---|
| 1 | MWRVVLRFLI IFILGEAVFP LRASESFSWE TSTCLTVLGI |
| | PFIDIILTTN |
| 51 | EDFVAQCGLQ IGTISSTNNA KIKEIFLIYK EKFPEASISF |
| | KRKEPLNLSQ |
| 101 | SHLSDLGILC MRNGETYAEG MANKENGPAL KQPKDLRLVL |
| | RCPNQPDTLL |
| 151 | YSEKEAEKGI ETNTCLCNQG YTLLDGQLIL YGDSIEKFLK |
| | ETKRRNNHTL |
| 201 | VDLCDSQVVT TFLGFFWSLL NYVQVLFLSE DSAKILAGIP |
| | DLAQATQLLS |
| 251 | HTVPLLFIYT NDSIHIIEQG KESSFTYNQD LTEPILGFLF |
| | GYINRGSMEY |
| 301 | CFNCAQSSLG ET* |

The cp6844 nucleotide sequence <SEQ ID 272> is:

| | |
|---|---|
| 1 | ATGTGGCGCG TTGTCCTCAG ATTCCTTATh ATTTTTATCT |
| | TGGGAAGAGC |
| 51 | CGTCTTCCCT CTAAGAGCTT CAGAAAGCTT CPCCTGGGAA |
| | ACATCGACCT |
| 101 | GTTTAACAGT GCTAGGGATT CCTTTCATAG ATATTATCCT |
| | CACAACGAAT |
| 151 | GAGGACTTTG TTGCCCAGTG CGGCCTGCAA ATAGGAACCA |
| | TTTCTTCGAC |
| 201 | TAATAACGCA AAAATAAAAG AAATTTTTTT GATATATAAG |
| | GAAAAATTTC |
| 251 | CAGAAGCCTC TATCAGTTTC AAACGAAAAG AACCTCTAAA |
| | CCTTTCCCAA |
| 301 | TCCCATCTCT CGGATTTAGG TATTTTATGT ATGCGTAACG |
| | GAGAAACTTA |
| 351 | CGCTGAGGGA ATGGCAAATA AAGAAAACGG ACCCGCTCTA |
| | AAACAACCCA |
| 401 | AGGATCTAAG ATTAGTTTTA CGTTGTCCTA ACCAACCAGA |
| | TACCCTGCTC |
| 451 | TACTCGGAAA AAGAAGCAGA AAAGGGCATA GAAACAAATA |
| | CTTGCCTATG |
| 501 | CAATCAGGGA TACACACPCC TGGATGGGCA ATTGATTCTC |
| | TACGGGGATA |
| 551 | GTATAGAAAA GTTTCTGAAA GAGACCAAAA GAAAGAATAA |
| | CCACACGCTT |
| 601 | GTTGATCTTT GTGACTCACA AGTCGTGACC ACGTTCCTCG |
| | GTCGCTTTTG |
| 651 | GTCTCTTCTA AACTACGTTC AAGTTCTTTT CCTATCTGAA |
| | GACTCCGCTA |
| 701 | AAATTCTTGC GGGCATCCCA GACCTAGCTC AAGCTACGCA |
| | ATTGCTTTCC |
| 751 | CACACCGTAC CTTTGCTTTT TATTTATACC AACGATTCTA |
| | TTCACATCAT |
| 801 | AGAACAAGGC AAAGAAAGTA GTTTTACCTA TAACCAAGAT |
| | TTAACAGAGC |
| 851 | CCATTTTAGG ATTTCTCTTT GGTTACATAA ATCGCGGCTC |
| | TATGGAATAC |
| 901 | TGCTTTAATT GTGCACAGTC TTCATTAGGA GAAACCTAA |

The PSORT algorithm predicts inner membrane (0.1786).

The protein was expressed in E. coli and purified as a GST-fusion product (FIG. 136A) and also in his-tagged form. The recombinant proteins were used to immunise mice, whose sera were used in a Western blot (FIG. 136B) and for FACS analysis.

These experiments show that cp6844 is a surface-exposed and immunoaccessible protein, and that it is a useful immunogen. These properties are not evident from the sequence alone.

### Example 137

The following C. pneumoniae protein (PID 4377201) was expressed <SEQ ID 273; cp7201>:

| | |
|---|---|
| 1 | VLVGICPSLY PEHPRSFYYR VSGDIGSRPD DRGFVNSGVE |
| | TLPYSSGSFG |
| 51 | IFWISFTDPT FNFAIVNTFM RTAGINEVSR PMTQDTETSL |
| | IEMRDLSEQQ |
| 101 | EANNTDSLEQ EESLMGIVGH TVGGVSMTVT SSPNIFYRIQ |
| | TLLGLPETLA |
| 151 | EAEENPTFPN STIDSLAEIM MNLVRISDAV SIFWIFPIVD |
| | TTYNGVLLAV |
| 201 | CIGFFGINGI CSTFLMLTNP RSRRDRWRNL RIMVLCYRSL |
| | GSGMNLFDLS |
| 251 | NNVRMAARRH VTSCTVALYA NVTLFGWTVA IQDALQYGPP |
| | SVRDAPYRYC |
| 301 | LRHRYCLTQR NEDSLQTTGT RFQVTRTHLE DQQMVASILN |
| | LSVFGLFFGF |
| 351 | VGLMTTFGGL EISPSCRWDA ANNRTVGIF* |

The cp7201 nucleotide sequence <SEQ ID 274> is:

| | |
|---|---|
| 1 | GTGCTCGTTG GTATCTGTCC TTCTCTATAT CCAGAACATC |
| | CTCGCTCCTT |
| 51 | TTATTATCGT GTTTCTGGAG ATATAGGCTC CCGATTCGAC |
| | GATAGAGGAT |
| 101 | TTGTAAACTC TGGAGTCGAA ACCCTGCCAT ACTCTTCAGG |
| | CAGCTTTGGG |
| 151 | ATTTTTTGGA TCTCGTTTAC GGATOCCACA TTTAATTTTG |
| | CTATCGTAAA |
| 201 | TACCTTTATG CGAACTGCAG GGATCAATGA AGTCTCTAGA |
| | CCCATGACAC |
| 251 | AAGATACAGA AACTTCATTG ATAGAAATGA GAGACCTAAG |
| | TGAACAACAA |
| 301 | GAAGCGAATA ACACAGATTC TTTAGAGCAA GAAGAGAGCT |
| | TAATGGGTAT |
| 351 | TGTAGGACAT ACTGTGGGAG GAGTTTCCAT GACCGTGACC |
| | TCCAGTCCAA |
| 401 | ATATCTTTTA TCGTATACAA ACACTTCTGG GACTGCCAGA |
| | GACTCTTGCA |
| 451 | GAAGCTGAAG AAAATCCTAC CTTCCCAAAT TCTACTATAG |
| | ATAGCCTTGC |
| 501 | AGAAATAATG ATGAACCTCG TAAGGATCTC TGATGCTGTC |
| | TCTATTTTCT |
| 551 | GGATTTTTCC TATCGTAGAT ACTACATATA ATGGAGTTTT |
| | ATTAGCCGTC |
| 601 | TGTATCGGCT TCTTCGGAAT CAATGGGATT TGTTCCACGT |
| | TCCTTATGCT |
| 651 | TACGAATCCA CGCTCTCGTC GAGATAGATG GAGGAATTTA |
| | CGCATCATGG |
| 701 | TTCTTTGCTA TCGTTCTTTG GGAAGCGGAA TGAATCTCTT |
| | TGATCTTAGC |
| 751 | AATAATGTGC GCATGGCAGC ACGTAGGCAT GTGACATCAT |
| | GTACAGTAGC |
| 801 | TCTCTATGCT ATGGTCACTC TATTTGGATG GACAGTAGCA |
| | ATACAAGATG |
| 851 | CTTTGCAATA TGGTTTCCCT AGCGTTCGGG ATGCCTTCTA |
| | TAGATATTGC |
| 901 | TTACGCCACA GATATTGCTT AACTCAAAGA AACGAAGACT |
| | CTCTGCAAAC |
| 951 | TACAGGAACG CGCTTTCAGG TTACCCGTAC ACATCTAGAA |
| | GATCAACTGA |
| 1001 | TGGTGGCTTC TATTTTGAAT TTGAGTGTTT TTGGGCTCTT |
| | TTTTGGATTC |
| 1051 | GTAGGGCTAA TGACCACGTT TGGAGGATTA GAAATCTCAC |
| | CATCTTGTCG |
| 1101 | GTGGGATGCA GCAAATAACC GAACGGTAGG TATTTTTTAG |

The PSORT algorithm predicts inner membrane (0.3102).

The protein was expressed in E. coli and purified as a GST-fusion product (FIG. 137A). The recombinant protein was used to immunise mice, whose sera were used in a Western blot (FIG. 137B) and for FACS analysis.

These experiments show that cp7201 is a surface-exposed and immunoaccessible protein, and that it is a useful immunogen. These properties are not evident from the sequence alone.

### Example 138

The following C. pneumoniae protein (PID 4377251) was expressed <SEQ ID 275; cp7251>:

| | |
|---|---|
| 1 | MAPIHGSNAF VEDIIMSHPS PQATYFSSTR AQKLHEFKDR |
| | HPVLTRIASV |
| 51 | IIKIFKVLIG LIILPLGIYW LCQTLCTNSI LPSKNLLKIF |
| | KKQPNTKTLK |
| 101 | TNYLHALQDY SSKNRVASMR RVPILQDNVL IDTLEICLSQ |
| | APTNRWMLIS |
| 151 | LGSDCSLEEI ACKEIFDSWQ RFAKLIGANI LVYNYPGVHS |
| | STGSBSLKDL |
| 201 | ASAHNICTRY LKDKEQGPGA KEIITYGYSL GGLIQAHALR |
| | DQKIVANDDT |
| 251 | TWIAVKDRCP LFISPEGEHS CRRIGKLVAR LFGWGTKAVE |
| | RSQDLPCLBZ |
| 301 | FLYPTDSLRR STVRQNKLLA PELTLAHAIR NSPYVQNKEF |
| | IEVRLSSDID |
| 351 | PIDSKTRVAL ATPILKKLS* |

The cp7251 nucleotide sequence <SEQ ID 276> is:

| | |
|---|---|
| 1 | ATGGCTCCAA TTCACGGAAG TAATGCGTTT GTTGAGGATA |
| | TTTTACATTC |
| 51 | CCACCCTTCT CCACAAGCGA CTTATTTTTC TTCAACACGC |
| | GCCCAAAAAC |
| 101 | TTCATGAGTT TAAAGACAGG CATCCCGTGC TTACACGGAT |
| | TGCTTCTGTA |
| 151 | ATTATTAAAA TTTTTAAAGT TCTGATAGGG CTGATCATCC |
| | TTCCCTTAGG |
| 201 | AATCTACTGG CTATGTCAAA CGCTTTGTAC AAACTCGATT |
| | CTCCCTTCCA |
| 251 | AGAATTTATT AAAAATTTTC AAGAAGCAAC CCAACACTAA |
| | AACCTTAAAA |
| 301 | ACTAATTATT TGCATGCTTT GCAAGATTAT TCCTCGAAAA |
| | ACCGCGTTGC |
| 331 | TTCCATGAGA CGAGTTCCTA TCCTCCAGGA TAATGTTCTC |
| | ATCGACACTT |
| 401 | TGGAAATATG CCTTTCACAA GCACCTACGA ATCGTTGGAT |
| | GCTCATTTCT |
| 451 | TTAGGAAGTG ACTGTAGCTT GGAAGAAATC GCTTGTAAGG |
| | AGATCTTTGA |
| 501 | TTCTTGGCAA AGATTTGCCA AGTTGATAGG GGCCAATATA |
| | CTCGTTTATA |
| 551 | ACTACCCCGG AGTCATGTCC AGCACAGGGA GCAGCAGCCT |
| | AAAGGACCTA |
| 601 | GCATCAGCTC ATAATATTTG TACAAGATAC CTTAAAGATA |
| | AAGAACAGGG |
| 651 | CCCTGGAGCA AAAGAAATCA TTACCTATGG GTACTCCCTA |
| | GGAGGTTTGA |
| 701 | TACAAGCAGA AGCATTGCCA GACCAGAAGA TTGTTGCAAA |
| | CGATGATACT |
| 751 | ACTTGGATAG CAGTCAAAGA TAGGTGTCCT CTCTTTATAT |
| | CTCCAAAAGG |
| 801 | TTTCCACAGT TGCAGACGCA TAGGAAAGCT AGTAGCTCGT |
| | CTTTTTGGCT |
| 851 | GGGGGACCAA AGCCGTAGAG AGAAGCCAAG ACCTTCCCTG |
| | CCTAGAAATT |
| 901 | TTTCTCTATC CTACGGATTC CTTACGAAGA TCAACAGTCA |
| | GACAGAACAA |
| 951 | GCTCTTAGCA CCTGAACTTA CTCTCGCTCA TGCGATAAAA |
| | AATAGTCCCT |
| 1001 | ATGTTCAAAA TAAAGAATTT ATAGAAGTAC GATTATCGTC |
| | TGATATCGAT |
| 1051 | CCCATCGACA GCAAAACAAG AGTGGCTCTT GCCACACCAA |
| | TTTTGAAAAA |
| 1101 | GCTCTCTTAG |

The PSORT algorithm predicts inner membrane (0.4545).

The protein was expressed in E. coli and purified as a GST-fusion product (FIG. 138A). The recombinant protein was used to immunise mice, whose sera were used in a Western blot (FIG. 138B) and for FACS analysis.

These experiments show that cp7251 is a surface-exposed and immunoaccessible protein, and that it is a useful immunogen. These properties are not evident from the sequence alone.

### Example 139

The following C. pneumoniae protein (PID 4377288) was expressed <SEQ ID 277; cp7288>:

| | |
|---|---|
| 1 | MHMSNPISLF SPAELIARYN LIPKTSPIYP RRTELIILEE |
| | NACQTRLTNV |
| 51 | AQVLHPSSLF SMSKKILNPC GCSGGPLCWV ELNILAFIIT |
| | SVLFIILLPV |
| 101 | NLIVAGLRLF MPLPPKKIVE DLSEPTTEET NEVIQPFIFA |
| | LQALLFEDNK |
| 151 | LRSFKIVEQS VGKAPLPNPF LNRLVAISPQ ESQEAMRKIP |
| | DLCSQLKKVL |
| 201 | KSLGVLTPEW KHMLKYFEGL KNEHDSNPDK KTFPILIKLL |
| | IEALTGKSSL |
| 251 | PKTPSTKEKM QAALFIASSC KTCKPTWGEV ITRSLNRLYS |
| | IANEGDNQLL |
| 301 | IWVQEFKERE LMSIQDGDDA EEYRFAAQQH GERYTEAIEQ |
| | VLRNESAAKL |
| 351 | QWHVINTMKF FHGKNLGLVT EHLQDTLGAL TLRQTTVDTH |
| | QGREDADLSA |
| 401 | ALFLNKYLNS GNQLVNSVFK SMQIADPETK ALIREFALDI |
| | LYASLELPOT |
| 451 | SAHTEVFSTL LMDPETYEPN KACIAYLLYV LKIIEL* |

The cp7288 nucleotide sequence <SEQ ID 278> is:

| | |
|---|---|
| 1 | ATGCATATGT CTAACCCCAT CTCTTTGTTT TCCCCTGCAG |
| | AGTTAATAGC |
| 51 | AAAGTACAAT TTAATTCCAA AAACTTCGCC GATTTATCCT |
| | CGGAGGACGG |
| 101 | AACTTATTAT CTTGGAAGAA AATGCGTGTC AAACACGCCT |
| | AACCAACGTG |
| 151 | GCTCAGGTCC TACATCCTTC TAGCCTATTC AGTATGTCAA |
| | AAAAAATACT |
| 201 | GAATCCCTGC GGGTGCTCTG GTGGTCCCTT ATGTTGGGTG |
| | ATTCTCAACA |
| 251 | TCCTAGCATT TATTATTACT TCAGTACTGT TTATCATTCT |
| | TTTACCGGTG |
| 301 | AATCTCATCG TAGCAGGTCT TCGTCTCTTC ATGCCTCTTC |
| | CCCCTAAAAA |
| 351 | AATCGTAGAG GATTTAAGTG AACCTACTAC TGAAGAAACG |
| | AATGAGGTCA |
| 401 | TTCAACCCTT CATTTTCGCT TTGCAAGCGT TGCTTTTTGA |
| | GGATAACAAA |
| 451 | CTTCGCTCTT TTAAAATTGT TGAACAAAGT GTAGGCAAAG |
| | CACCCTTACC |
| 501 | TAATCCCTTT TTAAATAGAC TAGTAGCAAT TTCGCCGCAA |
| | GAAAGCCAAG |
| 551 | AAGCCATGCG GAAGATTCCG GATCTATGCT CACAACTGAA |
| | AAAAGTATTA |
| 601 | AAGTCTCTAG GCGTGCTAAC TCCAGAATGG AAGCACATGC |
| | TGAAGTACTT |
| 651 | TGAGGGACTG AAAAACGAAC ATGATAGTAA TCCTGATAAA |
| | AAGACGTTCC |
| 701 | CAATATTGAT CAAGCTCCTC ATAGAACCTC TTACTGGAAA |
| | GTCCTCTTTA |
| 751 | CCCAAAACTC CTAGTACAAA GGAAAAAATG CAAGCGGCCT |
| | TATTTATTGC |
| 801 | AAGTTCTTGC AAGACTTGTA AGCCGACTTG GGGAGAAGTC |
| | ATAACCAGAT |
| 851 | CTCTTAACAG ACTCTATAGT ATAGCTAATG AAGGAGACAA |
| | TCAGCTTCTG |
| 901 | ATTTGGGTTC AAGAGTTTAA AGAACCAGAG CTGATGTCCA |
| | TCCAAGATGG |
| 951 | TGATGATGCT GAAGAGTATC GGTTTGCGGC TCAGCAACAC |
| | GGTGAGCGTT |
| 1001 | ACACAGAGGO AATAGAACAA GTTCTACGAA ACGAGTCAGC |
| | AGCCAAACTA |
| 1051 | CAATGGCATG TGATCAACAC TATGAAATTC TTCCATGGGA |
| | AAAATCTCGG |
| 1101 | TCTAGTTACA GAACACCTAC AAGATACTCT CGGCGCCCTA |
| | ACTTTACGTC |
| 1151 | AAACTACAGT GGACACACAT CAAGGCAGAG AAGACCCTGA |
| | TTTGTCAGCT |
| 1201 | GCTCTTTTCC TAAATAAGTA TTTAAATTCT GGAAATCAAC |
| | TTGTTAATAG |
| 1251 | CGTCTTTAAA TCCATGCAAA AAGCAGATCC AGAAACCAAA |
| | GCTTTAATCC |
| 1301 | GTGAGTTTGC TCTAGATATA TTATATGCAT CCTTACGGCT |
| | TCCTCAAACT |
| 1351 | TCCGCTCATA CCGAGGTCTT TTCTACACTC TTAAPGGACC |
| | CAGAGACCTA |
| 1401 | TGAACCTAAT AAAGCTTGTA TCGCCTACTT GCTCTATGTA |
| | TTAAAGATCA |
| 1451 | TCGAACTATA A |

The PSORT algorithm predicts inner membrane (0.5989).

The protein was expressed in E. coli and purified as a GST-fusion product (FIG. 139A). The recombinant protein was used to immunise mice, whose sera were used in a Western blot (FIG. 139B) and for FACS analysis.

These experiments show that cp7288 is a surface-exposed and immunoaccessible protein, and that it is a useful immunogen. These properties are not evident from the sequence alone.

### Example 140

The following C. pneumoniae protein (PID 4377359) was expressed <SEQ ID 279; cp7359>:

| | |
|---|---|
| 1 | MPGSVSSPPL SPVIVRERVP SSSGSDLIQP HAVLKISILI FALVTILGIV |
| 51 | LVVLSSALGA LPSYVLTVSG CIAIAVGLIG LGILVTRLIL STIRKVDAMG |
| 101 | YDAAVKEEQY LSRIRELESE NREIRDRNRA VEDQCAHLSE ENKDLRDPEY |
| 151 | LHGMTERLIA SLEIENQALV AENILLKDWN ASLSRDFRAY KQKFPLGALE |
| 201 | PWKEDIACIM EQNLFLKPEC IAMVKSLPLE TQRLFLYPKG FQSLVNRPAP |
| 251 | RSRFFQTPKY EYNSRNENED GKVAAVCARL KKEFFSAVLG ACSYEELGGI |
| 301 | CERAVALKET LPLPEAVYDT LVQEFPNLLT AESLWKEWCF YSYPYLRPYL |
| 351 | SVDYCKRLFV QLFEELCLKL FTTGSPEDQA LVRLFSYYRN HIPAVLASFG |
| 401 | LPPPETGGSV FVLLPKQENL LWSQIEVLAT RYLKDTFVRN SEWTGSFEMM |
| 451 | FSYNEMCKEI SEGRIRFAED YETRHSEEFP PSPLSBEGEG EEFLPPCSEE |
| 501 | EVSVLERPDL DVDSMWVWUP PVPKGPL* |

The cp7359 nucleotide sequence <SEQ ID 280> is:

| | |
|---|---|
| 1 | ATCCCAGGTT CTGTGTCATC ACCTCCTTTG TCTCCTGTAA TTGTCCGTGA |
| 51 | AAGGGTCCCA TCCTCTTCAG GATCCGACCT CATACAGCCT CATGCTGTTT |
| 101 | TAAAGATCTC CATCCTAATT TTTGCGCTTG TGACAATTTT AGGAATTGTT |
| 151 | CTTGTAGTGT TGTCTAGTGC TTTAGGAGCT CTTCCTAGTT TAGTTTTGAC |
| 201 | GGTTTCTGGT TGTATTGCAA TAGCTGTAGG CCTGATTGGT TTAGGGATTC |
| 251 | TTGTGACACG GCTGATTCTC TCTACGATCA GAAAAGTAGA TGCCATGGGT |
| 301 | TATGATGCTG CGGTCAAAGA AGAGCAGTAT TTGTCACGTA TCAGAGAATT |
| 351 | AGAGTCTGAA AATAGAGAGA TTAGAGATAG AAATCGTGCT GTCGAAGATC |
| 401 | AGTGTGCCCA TTTATCCGAA GAGAACAAGG ACCTTAGGGA TCCCGAATAT |
| 451 | CTACATGGAA TGACTGAAAG GCTCATTGCG AGCTTAGAAA TAGAGAATCA |
| 501 | AGCTCTCGTA GCTGAGAACA TTCTTCTCAA AGACTGGAAT GCAAGCCTAT |
| 551 | CTAGAGATTT CCGCGCATAT AAGCAAAAAT TTCCTCTTGG GGCATTAGAA |
| 601 | CCCTGGAAAG AAGATATTGC ATGTATCATG GAACAAAATC TCTTTTTAAA |
| 651 | ACCGGAATGT ATCGCGATGG TTAAGTCTCT TCCATTAGAG ACGCAACGGC |
| 701 | TGTTTTTATA TCCAAAAGGA TTTCAGTCTT TAGTTAATCG ATTTGCTCCG |
| 751 | CGGTCTCGCT TTTTCCAGAC TCCAAAGTAT GAATATAACA GTAGGAATGA |
| 801 | AAATGAGGAC GGAAAGGTAG CCGCAGTGTG CGCCCGTTTG AAAAAAGAAT |
| 851 | TCTTCAGTGC TGTTTTAGGA GCCTGTAGTT ACGAAGAACT ACGGGGCATT |
| 901 | TGTGAAAGAG CAGTAGCACT TAAAGAGACG TTGCCATTGC CTGAAGCTGT |
| 951 | CTATGATACC CTAGTTCAGG AGTTCCCAAA TCTTCTTACT GCTGAGAGTT |
| 1001 | TATGGAAAGA ATGGTGCTTC TATTCCTATC CCTACCTTCG TCCCTATCTT |
| 1051 | TCTGTGGATT ACTGTAAGAG GTTATTTGTA CAACTTTTTG AGGAACTCTG |
| 1101 | CCTAAAGCTT TTTACAACGG GATCTCCAGA AGACCAAGCT TTGGTTCCCC |
| 1151 | TTTTCTCTTA CTATAGGAAT CATATTCCCG CAGTCTTGGC CTCATTTGGT |
| 1201 | TTCCCCCCGC CTGAGACAGG GGGGTCTGTA TTTGTATTGC TACCAAAAGA |
| 1251 | AGAAAACCTT CTTTCGAGTC AAATTGAGGT GCTGGCTACA AGGTATCTCA |
| 1301 | AAGATACCTT CGTGAGAAAC TCAGAATGGA CGGGCTCTTT CGAGATGATG |
| 1351 | TTTTCTTATA ACGAGATGTG TAAGGAGATC TCCGAAGGAA GGATTCGTTT |
| 1401 | TGCTGAAGAC TATGAAACGA GGCATTCCGA AGAATTCCCT CCTTCCCCTC |
| 1451 | TCTCTGAAGA AGGAGAGGGC GAAGAATTCC TTCCTCCTTG CTCTGAAGAA |
| 1501 | GAGGTTTCGG TTCTTGAGCG CCCAGATCTA GATGTAGACT CTATGTGGGT |
| 1551 | CTGGCATCCG CCGGTCCCTA AGGGACCTCT TTAA |

The PSORT algorithm predicts inner membrane (0.7453).

The protein was expressed in E. coli and purified as a GST-fusion product (FIG. 140A). The recombinant protein was used to immunise mice, whose sera were used in a Western blot (FIG. 140B) and for FACS analysis.

These experiments show that cp7359 is a surface-exposed and immunoaccessible protein, and that it is a useful immunogen. These properties are not evident from the sequence alone.

### Example 141

The following C. pneumoniae protein (PID 4377374) was expressed <SEQ ID 281; cp7374>:

| | |
|---|---|
| 1 | MDKQSSGNSG CIWHPFTQSA LDSTPIKIVR GEGAYLYABS GTRYLDAISS |
| 51 | WWCNLHGHGH PYITKKLCEQ AQKLEHVIFA NFTHEPALEL VSKLAPLLPE |
| 101 | GLERFFFSDN GSTSIEIAMK IAVQYYYNQN KARSHFVGLS NAYHGDTFGA |
| 151 | MSIAGTSPTT VPFHDLFLPS STIAAPYYGK EELAIAQAKT VFSESNIAAF |
| 201 | IYEPLLQGAG GMLMYNPEGL KEILKLAKHY GVLCIADEIL TGFGRTGPLF |
| 251 | ASEPTDIPPD IICLSKGLTG GYLPLALTVT TKEIHDAFVS QDRMKALLHG |
| 301 | HTFTGNPLGC SAALASLDLT LSPBCLQQRQ MIERCHQEFQ EAHGSLWQRC |
| 351 | EVLGTVLALD YPAEATGYFS QYRDHLNRFF LEFGVLLRPL GNTLYVLPPY |
| 401 | CIQEEDLRII YSHLQDALCL QPQ* |

The cp7374 nucleotide sequence <SEQ ID 282> is:

| | |
|---|---|
| 1 | ATGGACAAGC AATCATCAGG GAATTCAGGG TGTATCTGGC ACCCCTTCAC |
| 51 | TCAATCTGCA TTAGATTCTA CACCCATAAA GATTGTAAGG GGAGAAGGTG |
| 101 | CTTACCTCTA TGCGGAATCA GGAACAAGAT ATCTTGATGC GATATCTTCA |
| 151 | TGGTGGTGCA ACCTCCACGG TCATGGGCAT CCCTACATTA CAAAAAAATT |
| 201 | ATGTGAGCAA GCACAGAAGT TAGAACATGT GATCTTCGCA AATTTCACCC |
| 251 | ATGAACCGGC TCTAGAGCTC GTATCGAAAC TCGCTCCCCT CCTTCCTGAA |
| 301 | GGTCTAGAAC GTTTCTTTTT CTCTGACAAC GGATCAACGT CTATCGAAAT |
| 351 | AGCAATGAAA ATTGCTGTGC AATATTACTA CAATCAAAAC AAGGCTAAGA |
| 401 | GCCATTTTGT TGGACTCAGC AATGCCTATC ACGCAGATAC ATTTGGAGCT |
| 451 | ATGTCGATAG CTGGCACGAG CCCTACTACA GTTCCCTTTC ATGATCTTTT |
| 501 | TCTTCCTTCC AGTACAATTG CTGCTCCCTA TTATGGCAAG GAAGAGCTTG |
| 551 | CCATTGCCGA AGCAAAAACA GTCTTTTCTG AAAGCAATAT CGCAGCGTTT |
| 601 | ATCTATGAGC CGCTATTGCA AGGTGCTGGA GGGATGTTAA TGTATAATCC |
| 651 | CGAAGGCCTA AAGGAGATTC TCAAGCTTGC CAAGCATTAC GGGGTTCTCT |
| 701 | GTATTGCTGA TGAAATTCTT ACTGGCTTTG GCCGTACGGG TCCACTGTTT |
| 751 | GCTTCTGAAT TTACAGACAT TCCTCCTGAC ATTATCTGTC TCCACTGTTT |
| 801 | TCTTACAGGA GGCTATCTCC CTCTAGCCTT GACAGTAACC ACTAAAGAAA |
| 851 | TTCATGATGC CTTTGTCTCC CAAGATCGGA TGAAGGCACT GCTTCATGGC |
| 901 | CATACCTTCA CAGGAAATCC TTTAGGCTGT AGTGCPGCCC TCGCTTCTTT |
| 951 | GGATCTCACC CTATCTCCAG AATGCCTACA AGAAAGGCAA ATGATAGAAC |
| 1001 | GGTGTCATCA AGAGTTTCAA GAAGCTCATG GTTCCCTATG GCAACGGTGT |
| 1051 | GAGGTTCTGG GCACGGTACT CGCTCTAGAT TACCCTGCAG AAGCTACAGG |
| 1101 | ATATTTTTCA CAATATAGAG ACCATCTCAA TCGCTTTTTC TTAGAACGTG |
| 1151 | GAGTCCTTCT TCGTCCTTTA GGGAACACAC TGTATGTGCT GCCCCCCTAC |
| 1201 | TGTATCCAAG AAGAAGATCT CCGGATTATT TATTCTCACC TACAGGATGC |
| 1251 | CCTATGTCTA CAACCACAGT AA |

The PSORT algorithm predicts cytoplasm (0.2930).

The protein was expressed in E. coli and purified as a GST-fusion product (FIG. 141A) and also in his-tagged form. The recombinant proteins were used to immunise mice, whose sera were used in a Western blot (FIG. 141B) and for FACS analysis.

These experiments show that cp7374 is a surface-exposed and immunoaccessible protein, and that it is a useful immunogen. These properties are not evident from the sequence alone.

### Example 142

The following C. pneumoniae protein (PID 4377377) was expressed <SEQ ID 283; cp7377>:

| | |
|---|---|
| 1 | MREETVSWSL EDIREIYHTP VFELIHKANA ILRSNFLHSE LQTCYLISIK |
| 51 | TGGCVEDCAY CAQSSRYHTH VTPEPMMKIV DVVERAKRAV ELGATRVCLG |
| 101 | AAWRNAKODR YFDRVLAMVK SITDLGABVC CALGMLSEEQ AKKLYDAGLY |
| 151 | AYNHNLDSSP EFYETIITTR SYEDRLNTLD VVNKSGISTC CQGXVGMGES |
| 201 | EEDRIKLLHV LATRDHIPES VPVNLLWPID GTPLQDQPPI SFWEVLRTIA |
| 251 | TARVVFPRSM VRLAAGRAFL TVEQQTLCFL AGANSIFYGD KLLTVENNDI |
| 301 | DEDAEMIKLL GLIPRPSFGI ERGNPCYANN S* |

The cp7377 nucleotide sequence <SEQ ID 284> is:

| | |
|---|---|
| 1 | ATGCGTGAAG AAACTGTATC CTGGTCATTA GAAGACATCC GCGAAATTTA |
| 51 | TCACACTCCC GTATTTGACC TGATTCACAA AGCCAATGCC ATATTGCGTA |
| 101 | GTAATTTCCT CCATTCAGAA CTGCAGACTT GCTATCTGAT TTCGATTAAA |
| 151 | ACTGGTGGAT GCGTTGAAGA TTGCGCCTAC TGTGCCCAAT CTTCCCGCTA |
| 201 | TCATACCCAC GTCACACCAG AACCTATGAT GAAAATTGTA GACGTTGTGG |
| 251 | AAAGGGCAAA ACGTGCTGTA GAGCTAGCCG CCACTCGTGT GTGTCTTGGG |
| 301 | GCTGCCTGGC GCAATGCTAA GGACGATCGA TACTTTGATA GAGTCCTCGC |
| 351 | TATGGTGAAA AGTATCACAG ATCTCGGAGC CGAGGTTTGT TGTGCTTTAG |
| 401 | GCATGCTCTC CGAAGAGCAA GCTAAAAAAC TGTATGATGC AGGACTTTAT |
| 451 | GCCTACAATC ATAATTTAGA CTCTTCTCCG GAATTCTATG AAACTATAAT |
| 501 | CACAACACGT TCTTATGAAG ATCGCCTCAA CACTCTTGAT GTAGTAAATA |
| 551 | AATCTGGCAT TAGTACATGC TGCGGTGGTA TTGTAGGTAT GGGAGAATCT |
| 601 | GAAGAAGACC GTATAAAGCT TCTTCATGTT CTTGCAACAA GAGATCATAT |
| 651 | CCCAGAATCC GTACCTGTAA ATTTACTTTG GCCGATTGAC GGCACGCCTT |
| 701 | TGCAAGACCA GCCTCCGATT TCTTTCTGGG AAGTCTTGCG AACCATAGCA |
| 751 | ACGGCACGGG TTGTTTTCCC CAGATCCATG GTACGACTTG CTGCAGGACG |
| 801 | CGCTTTCCTC ACAGTAGAAC AACAAACCTT ATGTTTTCTA GCCGGTGCCA |
| 851 | ACTCCATATT CTATGGAGAT AAACTGTTGA CTGTAGAAAA CAATGATATA |
| 901 | GATGAAGATG CTGAAATGAT CAAACTTTTA GGCTTAATCC CTCGCCCTTC |
| 951 | ATTTGGAATA GAAAGAGGTA ACCCATGTTA TGCCAACAAT TCCTAA |

The PSORT algorithm predicts cytoplasm (0.2926).

The protein was expressed in E. coli and purified as a GST-fusion product (FIG. 142A) and also in his-tagged form. The recombinant proteins were used to immunise mice, whose sera were used in a Western blot (FIG. 142B) and for FACS analysis.

These experiments show that cp7377 is a surface-exposed and immunoaccessible protein, and that it is a useful immunogen. These properties are not evident from the sequence alone.

### Example 143

The following C. pneumoniae protein (PID 4377407) was expressed <SEQ ID 285; cp7407>:

| | |
|---|---|
| 1 | MVCPNNSWPR MCGNFNCEWV EVTTTEETTR QSASDISEEA GSSGGAAPIT |
| 51 | TQPTKITKVE KRVQFNTAQG DESTIHMIQE AGELVDSILS HRRTQGCTEY |
| 101 | CYDSYATGCG QRCGSFGRLI CGTYKACCLD REDNQVAGLV HECEQTHGPI |
| 151 | AVALAAKTMG LNLMELVEKN TILSEEQKNE FRQHCSBAKT QLYGTMQSLS |
| 201 | QNFFLEGVNS IRERGLDDSL VQAVLSFIAT RSWEKTIESE EASGTSSASN |
| 251 | STRIPACYIL NTSPLTTSRL SCGSRDAPXP SSVGAEPQYV AKKYNDNGMA |
| 301 | RQLGKIQVTN LKTGDFSALG PFGLLIVKML NSFLLSASQS TSSILKHTGG |
| 351 | EICYTCPNFR DIVVLLMLAI GYCPANTDBT SVVDIHMIDD PIMTIFYELQ |
| 401 | YSYRTGRTSA SFLKKKPSLV RQESLDCPTP AESVPLMSSL EEEDENEDDD |
| 451 | EDGNLAYQQR ILECSGULQT LFLGIKINKE * |

The cp7407 nucleotide sequence <SEQ ID 286> is:

| | |
|---|---|
| 1 | ATGGTTTGCC CAAATAATTC TTGGTTCAGA ATGTGTGGAA ATTTCAACTG |
| 51 | CGAATGGGTT GAAGTAACAA CAACAGAAGA AACAACGCGG CAATCGGCTT |
| 101 | CAGATATAAG CGAAGAAGCT GGTTCGAGTG GAGGAGCTGC TCCTATAACT |
| 151 | ACGCAACCTA CTAAAATTAC AAAAGTAGAG AAACGTGTCC AATTTAATAC |
| 201 | TGCTCAAGGT GATGAAAGTA CAATAOACAT GATCCAAGAA GCAGGAGAAT |
| 251 | TGGTAGACTC CATTCTATCA CATAGACGAA CGCAAGGATG TACAGAGTAT |
| 301 | TGTTATGACA GTTACGCAAC TGGATGTGGT CAGCGTTGCG GATCTTTTGG |
| 351 | AAGACTCATT TGTGGAACGT ATAAAGCGTG TTGCTTAGAC AGAGAGGATA |
| 401 | ATCAGGTTGC TGGACTTGTC CATGAATGCG AACAGACCCA TGGTCCTATT |
| 451 | GCCGTTGCTT TAGCTGCTAA AACTATGGGC CTCAACTTAA TGGAACTTGT |
| 501 | AGAAAAAAAC ACTATTTTGT CTGAAGAACA GAAAAATGAA TTTAGACAGC |
| 551 | ATTGCTCGGA AGCTAAAACC CAACTCTATG GAACGATGCA GAGCCTTTCT |
| 601 | CAAAACTTTT TCCTTGAAGG AGTCAACAGC ATTAGAGAAC GCGGTCTAGA |
| 651 | CGATTCACTA GTCCAAGCCG TGCTAAGCTT TATTGCTACA AGGTCTTGGG |
| 701 | AAAAAACTAT AGAATCAGAG GAAGCCTCAG GAACATCTTC TGCTTCTAAT |
| 751 | TCTACACGCA TTCCTGCGTG CTATATCTTA AATACGAGCC CCTTAACGAC |
| 801 | GTCACCCCTA TCCTGTGGAT CAAGAGATGC GCGACGCCCA TCTTCAGTCG |
| 851 | GTGCAGAGCC CCAGTACGTA GCAAAAAAAT ACAATGACAA TGGCATGGCC |
| 901 | AGACAATTAG GAAAAATCCA AGTCACCAAT CTAAAAACAG GAGATTTTTC |
| 951 | AGCTTTAGGT CCTTTTGGTC TCCTGATTGT GAAAATGCTG AATAGCTTTC |
| 1001 | TCTTATCTGC ATCACAAAGC ACATCTTCTA TTCTAAAGCA CACAGGTGGA |
| 1051 | GAAATATGTT ATACGTGCCC AAATTTTCGT GATATCGTCG TTTTATTGAT |
| 1101 | GTPAGCGATT GGCTATTGCC CTGCAAATAC CGATGAGACA TCTGTCGTAG |
| 1151 | ATATACACAT GATAGATGAT CCGATTATGA CCATCTTCTA TCGACTACAA |
| 1201 | TACAGCTATA GAACAGGGAA AACTTCAGCA TCGTTTTTAA AAAAGAAACC |
| 1251 | CTCATTAGTA AGACAGGAAA GTCTTGATTG TCCTACCCCT GCAGAATCTG |
| 1301 | TCCCTCTCAT GTCAAGTCTC GAAGAAGAAG ATGAAAATGA AGATGATGAT |
| 1351 | GAGGATGGGA ATTTGGCGTA TCAACAGCGT ATCCTTGAAT GCTCGGGTCA |
| 1401 | TTTACAAACT CTATTTTTAG GGATAAAAAT AAACAAAGAA TAA |

The PSORT algorithm predicts inner membrane (0.1319).

The protein was expressed in E. coli and purified as a GST-fusion product (FIG. 143A). The recombinant protein was used to immunise mice, whose sera were used in a Western blot (FIG. 143B) and for FACS analysis.

These experiments show that cp7407 is a surface-exposed and immunoaccessible protein, and that it is a useful immunogen. These properties are not evident from the sequence alone:

### Example 144

The following C. pneumoniae protein (PID 4376432) was expressed <SEQ ID 287; cp6432>:

| | |
|---|---|
| 1 | MTRSTIESSD SLCSRSFSQK LSVQTLKNLC ERSLMKITSL VIAFLTLIVG |
| 51 | GALIALAGGO VLSFPLGLIL GSVLVLFSSX YLVSCCKFFT LKEMTMTCSV |
| 101 | KSKINIWFEK QRNKDIEKAL ENPDLFGENK RNVGNRSARN QLEMILHETD |
| 151 | GIILKRYNKG ARMYFYL* |

The cp6432 nucleotide sequence <SEQ ID 288> is:

| | |
|---|---|
| 1 | ATGACTAGAA GTACTATTGA AAGCAGTGAT TCGCTATGCT CAAGGTCTTT |
| 51 | TTCTCAAAAA TTAAGTGTCC AGACATTAAA AAATCTCTGT GAAAGTAGAT |
| 101 | TAATGAAGAT CACTTCTCTT GTGATTGCTT TCCTAACTCT AATTGTGGGG |
| 151 | GGTGCTCTTA TAGCTTTAGC AGGAGGGGGG GTTCTTTCTT TCCCTCTTGG |
| 201 | GCTAATCTTA GGAAGCGTAC TCGTTTTGTT TTCTTCTATC TATTTAGTCT |
| 251 | CTTGTTGTAA ATTTTTTACT TTAAAAGAGA TGACAATGAC CTGTAGTGTC |
| 301 | AAATCTAAAA TCAATATATG GTTTGAAAAG CAACGAAACA AAGACATCGA |
| 351 | AAAGGCATTA GAGAATCCAG ATCTCTTTGG AGAAAATAAG AGAAATGTTG |
| 401 | GAAATCGTTC GGCAAGAAAT CAACTAGAAA TGATCTTACA CGAGACTGAC |
| 451 | GGAATTATTT TGAAAAGATA TATGAAAGGA GCTAAAATGT ACTTTTATTT |
| 501 | ATGA |

The PSORT algorithm predicts inner membrane (0.5394).

The protein was expressed in E. coli and purified as a his-tagged product (FIG. 144A). The recombinant protein was used to immunise mice, whose sera were used in a Western blot (FIG. 144B) and for FACS analysis.

These experiments show that cp6432 is a surface-exposed and immunoaccessible protein, and that it is a useful immunogen. These properties are not evident from the sequence alone.

### Example 145

The following C. pnuemoniae protein (PID 4376433) was expressed <SEQ ID 289; cp6433>:

| | |
|---|---|
| 1 | MNWVPKTIDH VDPESEIDIR KVVSCYKLIK ECQPEFRSLI SELLGVIRCG |
| 51 | LRLLKRSKYQ EQARTVSDED APLFCLTRSY YQDGYLTPLR AGPRDLINHY |
| 101 | IHLRRRENPK HFFSPKHPCY YARLAFNESV CVYRELFDIE RLTKMYVEGD |
| 151 | YSKEQEKNLQ AILSFVKTLD EGKDFLIEHK DTDLIGRGFT DVFCT* |

The cp6433 nucleotide sequence <SEQ ID 290> is:

| | |
|---|---|
| 1 | ATGAATTGGG TTCCAAAAAC AATAGACCAT GTAGATCCAG AATCAGAGAT |
| 51 | AGATATACOT AAAGTCGTCT CCTGCTATAA GTTGATAAAA GAATGTCAAC |
| 101 | CTGAATTTCG ATCTCTTATA AGTGAATTAC TAGGAGTGAT TCGGTGTGGC |
| 151 | TTAAGACTAT TAAAACGTTC TAAGTATCAA GAACAGGCTA GAACTGTATC |
| 201 | TGATGAAGAT GCACCTCTTT TCTGCCTGAC TCGTTCTTAT TATCAAGATG |
| 251 | GTTATCTCAC GCCATTAAGA GCAGGACCTC GTGATCTTAT AAATCACTAT |
| 301 | ATACACTTGC GTCGCCGAGA GAATCCTAAG CATTTTTTCA GTCCTAAGCA |
| 351 | TCCATGTTAT TATGCTCGAT TGGCTTTTAA TGAGTCAGTG TGTGTCTATA |
| 401 | GAGAACTCTT TGATATAGAG CGACTTACAA AAATGTATGT CGAGGGTGAT |
| 451 | TATTCTAAAG AACAAGAGAA AAACCTACAG GCTATTCTTA GTTTTGTGAA |
| 501 | AACTCTAGAT GAAGGAAAGG ACTTTCTTAT TGAACATAAA GATACCGATC |
| 551 | TCATTGGGAG AGGTTTTACT GATGTGTTCT GCACTTAA |

The PSORT algorithm predicts cytoplasm (0.4068).

The protein was expressed in E. coli and purified as a his-tagged product (FIG. 145A). The recombinant protein was used to immunise mice, whose sera were used in a Western blot (FIG. 145B) and for FACS analysis.

These experiments show that cp6433 is a surface-exposed and immunoaccessible protein, and that it is a useful immunogen. These properties are not evident from the sequence alone.

### Example 146

The following C. pneumoniae protein (PID 4376643) was expressed <SEQ ID 291; cp6643>:

| | |
|---|---|
| 1 | MGYLPVSATD VLFESPAAPL INSANTQNQK LIELKGKQQA ESSPRTITSV |
| 51 | ILBVLLVIGC CLIVLSLLAI RPALQFTLET GHPAAIAVLA VSGTILLVAV |
| 101 | IILFCFLAAV PFAAKKTYKY VKTVDDYASW HSHQQTPTLG TIFSGIVYAE |
| 151 | SQAQL* |

The cp6643 nucleotide sequence <SEQ ID 292> is:

| | |
|---|---|
| 1 | ATGGGATATC TTCCAGTATC TGCTACGGAC GTTCTTTTTG AAAGTCCAGC |
| 51 | CGCTCCCTTA ATCAATAGCG CAAACACACA AAATCAGAAA CTCATAGAAC |
| 101 | TCAAGGGGAA GCAGCAAGCT GAGTCTTCTC CACGGACAAT CACTTCTGTC |
| 151 | ATATTGGAAG TTCTCCTAGT GATCGGATGC TGCCTCATAG TTCTTAGTTT |
| 201 | ATTGGCAATC CGCCCTGCTC TGCAATTCAC TCTAGAAACT GGACATCCAG |
| 251 | CTGCCATTGC AGTCCTTGCT GTCTCAGGAA CAATTCTATT GGTGGCTGTT |
| 301 | ATCATCTTGT TTTGCTTTCT AGCAGCTGTG CCATTCGCTG CTAAGAAAAC |
| 351 | TTATAAATAT GTTAAGACGG TTGATGACTA TGCTTCTTGG CATTCTCATC |
| 401 | AGCAAACACC GACCCTAGGC ACTATCTTTT CAGGTATCGT CTATGCAGAA |
| 451 | TCCCAGGCGC AATTATAG |

The PSORT algorithm predicts inner membrane (0.6859).

The protein was expressed in E. coli and purified as a his-tagged product (FIG. 146A). The recombinant protein was used to immunise mice, whose sera were used in a Western blot (FIG. 146B) and for FACS analysis.

These experiments show that cp6643 is a surface-exposed and immunoaccessible protein, and that it is a useful immunogen. These properties are not evident from the sequence alone.

### Example 147

The following C. pneumoniae protein (PID 4376722) was expressed <SEQ ID 293; cp6722>:

| | |
|---|---|
| 1 | VSSTLNGVFP SSLPEESADL FITNKEIVAL GEKGNVFLTH SIPMHIAAIT |
| 51 | ILVIVALAGI AIICLGCYSQ SILLIAVGIV LTILTLLCLQ ALVGGIKFIR |
| 101 | QLPQQLHTTV QFIREKIRPE SSLQLVTNAQ RKTTQDTLKL YEELCDLSQK |
| 151 | EFKLQSTLYQ KRPELSHKNE RTNQN* |

The cp6722 nucleotide sequence <SEQ ID 294> is:

| | |
|---|---|
| 1 | GTGTCTAGTA CTTTAAACGG GGTATTTCCC TCATCCCTTC CGGAAGAGTC |
| 51 | TGCTGATTTA TTCATTACGA ATAAGGAGAT CGTAGCTTTG GGGGAGAAGG |
| 101 | GCAATGTTTT TCTCACCCAC TCCATTCCTA TGCATATTGC TGCGATTACG |
| 151 | ATCTTAGTGA TTGTAGCTCT TGCTGGAATC GCTATTATCT GTTTGGGTTG |
| 201 | CTATAGCCAA AGCATTCTGT TGATTGCCGT TGGCATTGTT CTTACTATTT |
| 251 | TGACTCTTCT CTGCCTACAA GCCTTGGTAG GATTTATTAA ATTCATCCGG |
| 301 | CAGCTCCCTC AGCAGCTCCA TACGACAGTA CAATTTATCA GGGAGAAGAT |
| 351 | TCGACCTGAA TCCTCTCTAC AGCTTGTAAC CAATGCACAG AGAAAAACCA |
| 401 | CTCAAGATAC GCTAAAGTTA TACGAAGAAC TCTGCGACCT CTCACAAAAA |
| 451 | GAGTTCAAAC TGCAATCAAC TCTTTATCAA AAACGTTTTG AGCTTTCTCA |
| 501 | CAAGAATGAA AAGACAAATC AAAACTAG |

The PSORT algorithm predicts inner membrane (0.6668).

The protein was expressed in E. coli and purified as a his-tagged product (FIG. 147A). The recombinant protein was used to immunise mice, whose sera were used in a Western blot (FIG. 147B) and for FACS analysis.

These experiments show that cp6722 is a surface-exposed and immunoaccessible protein, and that it is a useful immunogen. These properties are not evident from the sequence alone.

### Example 148

The following C. pneumoniae protein (PID 4377253) was expressed <SEQ ID 295; cp7253>:

| | |
|---|---|
| 1 | MSELAPCSTG LQMVPHTQVH HALDTRRVIL TIAACLSLIA GIVLVGLGAA |
| 51 | AILPSLFGVI GGMIIILFSS IALIYLYKKT RBVDQIALEP LPEMISKDQS |
| 101 | IIDFVKTRDY ASLEKXATFA YTHTHYYDGS MVFYREIPRF MLGSYLALRK |
| 151 | DNDRQALF* |

The cp7253 nucleotide sequence <SEQ ID 296> is:

| | |
|---|---|
| 1 | ATGAGCGAGC TCGCCCCCTG CTCGACAGGA TTGCAGATGG TCCCCCATAC |
| 51 | GCAGGTCCAT CATGCCCTTG ATACGCGGAG AGTCATTCTA ACGATAGCCG |
| 101 | CCTGTCTGTC TTTAATTGCA GGAATCGTGT TGGTTGGCTT AGGTGCTGCA |
| 151 | GCAATCCTGC CCTCGCTTTT TGGAGTCATT GGAGGAATGA TTCTTATTCT |
| 201 | GTTTTCTTCG ATCGCCCTCA TTTATTTATA CAAGAAGACA AGGGAGGTGG |
| 251 | ATCAGATTGC TCTGGAGCCT CTTCCTGAGA TGATTTCTAA AGATCAAAGC |
| 301 | ATTATAGATT TTGTAAAGAC ACGAGACTAT GCATCTTTAG AAAAGAAAGC |
| 351 | GACCTTTGCT TATACTCATA CTCATTATTA CGATGGAAGC ATGGTCTTCT |
| 401 | ATAGGGAGAT CCCTAGATTT ATGTTAGGCT CTTATCTCGC GCTTCGCAAA |
| 451 | GACATGGACC GCCAAGCTCT TTTTTGA |

The PSORT algorithm predicts inner membrane (0.5394),

The protein was expressed in E. coli and purified as a his-tagged product (FIG. 148A). The recombinant protein was used to immunise mice, whose sera were used in a Western blot (FIG. 148B) and for FACS analysis.

These experiments show that cp7253 is a surface-exposed and immunoaccessible protein, and that it is a useful immunogen. These properties are not evident from the sequence alone.

### Example 149

The following C. pneumoniae protein (PID 4376264) was expressed <SEQ ID 297; cp6264>:

| | |
|---|---|
| 1 | VISGLLFLLV RRRVPTVRSE EIPRGVSVTP SEEPALBKAQ KEPETKKILD |
| 51 | RLPKELDQLD TYIQEVDACL ERLRDPKYED RGLLTEAXEK LRVFDVVEIG |
| 101 | MMSEFLDIQR VLNEEAYYVE HCQDPLENIA YBIFSSQELP DYYCAGVCGY |
| 151 | LPSGDARADR LKRSVKEVMD RFMRVTWKSW EASVMLDHSY GVARELFKKA |
| 201 | VGVLEESVYK ILFKSYRDAF YECEKMCIQR DGRFKWL* |

The cp6264 nucleotide sequence <SEQ ID 298> is:

| | |
|---|---|
| 1 | GTGATTTCGG GACTTCTATT CCTTCTAGTA AGACGAGAGG TTCCGACAGT |
| 51 | ACGTTCAGAG GAAATTCCCA GAGGGGTTTA TGTGACCCCT TCTGAAGAGC |
| 101 | CTGCTCTAGA GAAGGCTCAA AAAGAACCGG AGACAAAGAA AATTTTAGAT |
| 151 | CGGTTGCCGA ACGAATTGGA TCAGTTAGAT ACGTATATTC AGGAAGTGTT |
| 201 | TGCATGTTTA GAGAGGCTGA AGGATCCTAA GTACGAAGAT CGAGGTCTTT |
| 251 | TAACAGAGGC GAAGGAGAAA CTTCGAGTTT TTGACGTTGT TGAGAAAGAT |
| 301 | ATGATGTCAG AGTTTTTAGA CATACAACGA GTGTTGAATG AGGAAGCATA |
| 351 | TTATGTAGAA CATTGTCAAG ATCCCCTAGA GAATATAGCC TACGAGATTT |
| 401 | TCTCTTCCCA AGAGCTTCGT GATTACTACT GTGCAGGGGT GTGTGGGTAT |
| 451 | TTGCCTTCTG GGGATGCTCG AGCGGATCGA TTAAAGAGAT CAGTTAAGGA |
| 501 | GGTAATGGAT CGCTTTATGA GGGTGACCTG GAAATCTTGG GAGGCATCAG |
| 551 | TCATGTTGGA TCATAGCTAT GGGGTAGCGC GAGAGTTATT CAAGAAGGCA |
| 601 | GTAGGAGTAC TAGAGGAGAG TGTCTATAAA ATTCTGTTTA AGAGCTATAG |
| 651 | AGATGCGTTT TATGAATGTG AGAAGGCAAA GATCCAGAGG GATGGGCGTT |
| 701 | TCAAATGGTT ATAG |

The PSORT algorithm predicts cytoplasm (0.2817).

The protein was expressed in E. coli and purified as a his-tagged product (FIG. 149A). The recombinant protein was used to immunise mice, whose sera were used in a Western blot (FIG. 149B) and for FACS analysis.

These experiments show that cp6264 is a surface-exposed and immunoaccessible protein, and that it is a useful immunogen. These properties are not evident from the sequence alone.

### Example 150

The following C. pneumoniae protein (PID 4376266) was expressed <SEQ ID 299; cp6266>:

| | |
|---|---|
| 1 | MLLLISGALF LTLGIPGLSA AISFGLGIGL SALGGVLMIS GLLCLLVKRE |
| 51 | IPTVRPEEIP EGVSLAPSEE PALQAAQKTL AQLPKELDQL DTDIQEVFAC |
| 101 | LRKLKDSKYE SRSFLNDAKK ELRVFDFVVE DTLSEIFELR QIVAQEGWDL |
| 151 | NFLINGGRSL MMTAESESLD LFHVSKELGY LPSGDVRGEG LKKSAKEIVA |
| 201 | RLMSLHCEIH KVAVAFDRNS YAMAEKAFAK ALGALEESVY RSLTQSYRDK |
| 251 | FLESERAKIP WNGHITWLRD DAKSGCAEKK LGMPRNVGRN LGKQSFG* |

The cp6266 nucleotide sequence <SEQ ID 300> is:

| | |
|---|---|
| 1 | ATGCTCTTAC TGATTTCAGG AGCTCTCTTT CTGACGTTAG GGATTCCAGG |
| 51 | ATTGAGTGCA GCAATTTCTT TTGGATTAGG CATCGGTCTC TCCGCATTAG |
| 101 | GAGGAGTGCT GATGATTTCG GGACTACTAT GTCTTTTAGT AAAACGAGAG |
| 151 | ATTCCGACAG TACGACCAGA AGAAATTCCT GAAGGGGTTT CGCTGGCTCC |
| 201 | TTCTGAGGAG CCAGCTCTAC AGGCAGCTCA GAAGACTTTA GCTCAGCTGC |
| 251 | CTAAGGAATT GGATCAGTTA GATACAGATA TTCAGGAAGT GTTCGCATGT |
| 301 | TTAAGAAAGC TGAAACATTC TAAGTATGAA AGTCGAAGTT TTTTAAACGA |
| 351 | TGCTAAGAAG GAGCTTCGAG TTTTTCACTT TGTGGTTGAG GATACCCTCT |
| 401 | CGGAGATTTT CGAGTTGCGG CAGATTGTGG CTCAAGAGGG ATGGGATTTA |
| 451 | AACTTTTTGA TCAATGGGGG ACGAAGCCTC ATGATGACTG CAGAATCTGA |
| 501 | ATCGCTTGAT TTGTTTCATG TATCGAAGCG GCTAGGGTAT TTACCTTCTG |
| 551 | GGGATGTTCG AGGGGAGGGG TTAAAGAAAT CTGCGAAGGA GATAGTCGCT |
| 601 | CGTTTGATGA GCTTGCATTG CGAGATTCAC AAGGTGGCGG TAGCGTTTGA |
| 651 | TAGGAATTCC TATGCGATGG CAGAAAAGGC GTTTGCGAAA GCGTTGGGAG |
| 701 | CTTTAGAAGA GAGTGTGTAT CGGAGTCTGA CGCAGAGTTA TAGAGATAAA |
| 751 | TTTTTGGAGA GCGAGAGGGC GAAGATCCCA TGGAATGGGC ATATAACCTG |
| 801 | GTTAAGAGAT GATGCGAAGA GTGGGTGTGC TGAAAAGAAG CTCGGGATGC |
| 851 | CGAGGAACGT TGGAAGAAAT TTAGGAAAGC AGTCTTTTGG GTAG |

The PSORT algorithm predicts inner membrane (0.3590).

The protein was expressed in E. coli and purified as a his-tag product (FIG. 150A). The recombinant protein was used to immunise mice, whose sera were used in a Western blot (FIG. 150) and for FACS analysis.

These experiments show that cp6266 is a surface-exposed and immunoaccessible protein and that they it is a useful immunogen. These properties are not evident from the sequence alone.

### Example 151

The following C. pneumoniae protein (PID 4376895) was expressed <SEQ ID 301; cp6895>:

| | |
|---|---|
| 1 | MKIKKSFQYS LCQAKRFQNM LPNHFDPCLQ PVNLQLKQDR LAYGELIILL |
| 51 | SKYQQKTFSS LLKEETCSLN RAKQHLLYKI LRDFNTMQHL RSLGLNGWGE |
| 101 | IPMSPCL* |

The cp6895 nucleotide sequence <SEQ ID 302> is:

| | |
|---|---|
| 1 | ATGAAGATTA AAAAATCTTT TCAATACAGT TTATGCCAAG CAAAGAGATT |
| 51 | TCAGAACATG CTGCCAAACC ACTTTGATCC ATGTTTGCAG CCAGTGAATT |
| 101 | TACAACTCAA ACAAGACAGA TTGGCATACG GGGAGCTCAT CATATTGCTA |
| 151 | TCTAAATATC AACAAAAGAC CTTTTCCTCT TTGTTGAAGG AAGAAACATG |
| 201 | TTCTCTTAAT CGTGCGAAGC AGCACTTATT GTATAAGATT TTGAGAGATT |
| 251 | TTAATAOPAT GCAGCATCTA AGGTCCCTCG GATTAAATGG TTGGGGAGAG |
| 301 | ATCCCTATGA GTCCTTGCCT CTAA |

The PSORT algorithm predicts cytoplasm (0.3264).

The protein was expressed in E. coli and purified as a his-tag product (FIG. 151A). The recombinant protein was used to immunise mice, whose sera were used in a Western blot (FIG. 151B) and for FACS analysis.

These experiments show that cp6895 is a surface-exposed and immunoaccessible protein and that it is a useful immunogen. These properties are not evident from the sequence alone.

### Example 152 and Example 153

The following C. pneumoniae protein (PID 4376282) was expressed <SEQ ID 303; cp6282>:

| | |
|---|---|
| 1 | MSLLNLPSSQ DSASEDSTSQ SQIFDPIRNR ELVSTPEEKV RQRLLSFLMH |
| 51 | KLNYPKKLII IEKELKTLFP LLMRKGTLIP KRRPDILIIT PPTYTDAQGN |
| 101 | THNLGDPRPL LLIECKALAV NQNALKQLLS YNYSIGATCI AMAGKHSQVS |
| 151 | ALFNPKTQTL DFYPGLPEYS QLLNYFISLN L* |

The cp6282 nucleotide sequence <SEQ ID 304> is:

| | |
|---|---|
| 1 | ATGTCCTTAT TGAACCTTCC CTCAAGCCAG GATTCTGCAT CTGAGGACTC |
| 51 | CACATCGCAA TCTCAAATCT TCGATCCCAT TAGAAATCGG GAGTTAGTTT |
| 101 | CTACTCCCGA AGAAAAAGTC CGCCAAAGGT TGCTCTCCTT CCTAATGCAT |
| 151 | AAGCTGAACT ACCCTAAGAA ACTCATCATC ATAGAAAAAG AACTCAAAAC |
| 201 | TCTTTTTCCT CTGCTTATGC GTAAAGGAAC CCTAATCCCA AAACGCCGCC |
| 251 | CAGATATTCT CATCATCACT CCCCCCACAT ACACAGACGC ACAGGGAAAC |
| 301 | ACTCACAACC TAGGCGACCC AAAACCCCTG CTACTTATCG AATGTAAGGC |
| 351 | CTTAGCCGTA AACCAAAATG CACTCAAACA ACTCCTTAGC TATAACTACT |
| 401 | CTATCGGAGC CACCTGCATT GCTATGGCAG GGAAACACTC TCAAGTGTCA |
| 451 | GCTCTCTTCA ATCCAAAAAC ACAAACTCTT GATTTTTATC CTGGCCTCCC |
| 501 | AGAGTATTCC CAACTCCTAA ACTACTTTAT TTCTTTAAAC TTATAG |

The PSORT algorithm predicts cytoplasm (0.362).

The following C. pneumoniae protein (PID 4377373) was also expressed <SEQ ID 305; cp7373>:

| | |
|---|---|
| 1 | MSTTTVKHFI HTASRWEPVL KEIVASNYWH AQWINTLSFL ENSGAKKISA |
| 51 | SEHPTEVKEE VLKHAAEEFR HGHYLKTQIS RISETSLPDY TSKNLLGGLL |
| 101 | TKYYLHLLDL RTCRVLENEY SLSGQTLKTA AYILVTYAIE LHASELYPLY |
| 151 | HDILKEAQSK ITVKSIILEE QGHLQEMERE LKDLPHGEEL LGYACQFEGE |
| 201 | LCLQFVERLE QMIFDPSSTF TKF* |

The cp7373 nucleotide sequence <SEQ ID 306> is:

| | |
|---|---|
| 1 | ATGTCTACAA CCACAGTAAA ACACTTTATC CACACAGCCT CTCGTTGGGA |
| 51 | GCCCGTTCTC AAAGAGATCG TAGCTTCCAA CTATTGGCAT GCACAATGGA |
| 101 | TAAATACCCT GTCCTTTTTA GAAAATAGTG GAGCAAAAAA AATCTCCGCA |
| 151 | AGTGAACATC CTACGGAGGT AAAGGAAGAA GTTTTAAAAC ATGCTGCTGA |
| 201 | AGAATTTCGT CATGGTCACT ATCTAAAAAC TCAGATTTCT AGAATCTCAG |
| 251 | AGACTTCTCT CCCIGACTAT ACATCTAAAA ATCTTCTGGG AGGCTTACTT |
| 301 | ACAAAATATT ACCTCCATCT TCTAGATWTA AGGACGTGCC GAGTACTGGA |
| 351 | AAATGAATAC TCCCTATCGG GACAAACGTT AAAAACTGCA GCGTATATTT |
| 401 | TAGTTACCTA CGCAATCGAA CTTCGTGCTT CTGAACTTTA TCCTCTGTAT |
| 451 | CACGATATTC TGAAAGAAGC TCAAAGTAAA ATAACGGTAA AATCCATTAT |
| 501 | CTTAGAAGAG CAAGGCCATC TGCAAGAGAT GGAACGTGAA CTTAAAGATC |
| 551 | TCCCCCACGG GGAGGAACTC TTAGGCTATG CTTGCCAATT CGAAGGGGAG |
| 601 | CTTTGCTTGC AGTTTGTAGA GAGATTAGAA CAAATGATCT TCGATCCTTC |
| 651 | CTCGACTTTT ACAAAGTTCT AG |

The PSORT algorithm predicts cytoplasm (0.1069).

The proteins were expressed in E. coli and purified as his-tag products (FIG. 152A; 6282 lanes 8 & 9; 7373=lanes 24). The recombinant proteins were used to immunise mice, whose sera were used in Western blots (FIGS. 152B & 153) and for FACS analysis.

These experiments show that cp6282 & cp7373 are surface-exposed and immunoaccessible proteins and that they are useful immunogens. These properties are not evident from the sequence alone.

### Example 154, Example 155, Example 156, Example 157 and Example 158

The following C. pneumoniae protein (PID 4376412) was expressed <SEQ ID 307; cp6412>:

| | |
|---|---|
| 1 | MSSSEVVFQT VHGLGFGGLS SKSVVPFKKS LSDAPRVVCS ILVLTLGLGA |
| 51 | LVCGIAITCW CVPGVILMGG ICAIVLGAIS LALSLFWLWG LFSNCCGSKR |
| 101 | VLPGEGLLRD KLLDGGFSRA APSGMGLPGD GSPRASTPSC LEELQAEIQA |
| 151 | VTQAIDQMSD D* |

The cp6412 nucleotide sequence <SEQ ID 308> is:

| | |
|---|---|
| 1 | ATGAGCAGTT CGGAAGTTGT TTTCCAGACA GTTCATGOCC TTGGCTTTGG |
| 51 | TGGATTGTCT TCAAAAAGTG TTGTCCCTTT TAAGAAAAGT CTTTCGGATG |
| 101 | CGCCCCGTGT TGTGTGCTCG ATTTTAGTTT TGACTCTGGG CTTGGGAGCG |
| 151 | CTTGTTTGTG GTATTGCCAT TACTTGTTGG TGTGTCCCGG GAGTTATTTT |
| 201 | AATGGGGGGA ATTTGCOCTA TAGTTTTAGG TGCAATTTCT TTAGCTTTAA |
| 251 | GTCTATTTTC GTTGTGGGGT TTATTTTCTA ATTGTTGTGG TTCTAAGAGA |
| 301 | GTTTTACCGC GTGAGGGATT GCTACGGGAT AAGCTTTTAG ATGGTGGATT |
| 351 | TTCAAGAGCG GCACCTTCAG GAATGGGACT TCCGGGTGAT GGATCTCCAA |
| 401 | GAGCGTCAAC GCCATCTTGC CTAGAGGAAC TTCAAGCAGA GATACAGGCA |
| 451 | GTTACTCAAG CTATCGATCA GATGTCAGAT GATTGA |

The PSORT algorithm predicts inner membrane (0.4864).

The following C. pneumoniae protein (PID 4376431) was also expressed <SEQ ID 309; cp6431>:

| | |
|---|---|
| 1 | LRAGGSLVTT YPKEGQRLRS PEQLRVLDDL VQSYPNHLHA IELDCGAIPQ |
| 51 | DLIGATYIIT FADFSTYILS LRSYQANSPS DDTWGIWFGS IDDPVQAVIS |
| 101 | FLKDHGFALP STLAQDPLLC TNK* |

The cp6431 nucleotide sequence <SEQ ID 310> is:

| | |
|---|---|
| 1 | TTGCGAGCAG GAGGTAGTCT TGTTACAACA TACCCTAAGG AAGGTCAGAG |
| 51 | ATTGCGCTCC CCAGAACAGT TAAGAGTTCT GGATGATTTA GTGCAAAGCT |
| 101 | ATCCAAATCA CCTACATGCG ATTGAACTTG ATTGTGGTGC AATCCCTCAA |
| 151 | GATTTGATCG GAGCCACCTA TATCATCACG TTCGCCGATT TTTCCACCTA |
| 201 | TATTCTCTCT TTAAGAAGCT ACCAAGCCAA TTCTCCCTCC GATGATACAT |
| 251 | GGGGGATTTG GTTTGGATCT ATTGACGATC CTGTTCAAGC AGTCATATCA |
| 301 | TTTTTAAAAG ATCATGGATT TGCTCTTCCC TCGACCTTAG CTCAAGATCC |
| 351 | TTTGCTTTGT ACTAACAAGT AA |

The PSORT algorithm predicts cytoplasm (0.2115).

The following C. pneumoniae protein (PID 4376443) was also expressed <SEQ ID 311; cp6443>:

| | |
|---|---|
| 1 | MIMTTISNSP SPALNPELSL IPPPTLVSSG TQTSLAYTIP AQGRRSTLRI |
| 51 | ILDIFIIILG LATIISTFIV IFFLNGLNLL STPSIISSSC LIIVGLLFLI |
| 101 | MGLYFMISSL DQGLVGLLQK ELSQAEEREE EYIQEIEALR GAPRAESPTE |
| 151 | SPSTWL* |

The cp6443 nucleotide sequence <SEQ ID 312> is:

| | |
|---|---|
| 1 | ATGATTATGA CTACTATATC TAACTCACCC TCCCCTGCAT TGAATCCCGA |
| 51 | ACTTTCCCTT ATTCCTCCAC CAACACTTGT ATCTTCAGGT ACGCAAACAT |
| 101 | CTCTAGCTTA TACGATCCCC GCACAAGGAC GAAGATCCAC CCTACGTATT |
| 151 | ATATTAGATA TATTCATTAT CATTCTTGGT TTAGCTACGA TCATTTCTAC |
| 201 | CTTTATTGTT ATTTTCTTTT TAAATGGGCT GAACTTGCTC TCGACCCCAT |
| 251 | CTATTATCTC TTCGTCATGT TTAATCATTG TTGGATTGCT TTTTTTGATT |
| 301 | ATGGGGTTAT ATTTCATGAT CTCGAGTTTG GATCAGGGGC TTGTAGGCCT |
| 351 | TCTGCAAAAG GAACTCTCTC AAGCCGAAGA AAGAGAAGAA GAGTATATCC |
| 401 | AGGAAATCGA AGCTTTAAGA GGAGCTCCTA GAGCAGAATC TCCCACAGAG |
| 451 | TCTCCTAGTA CCTGGTTATG A |

The PSORT algorithm predicts inner membrane (0.5585).

The following C. pneumoniae protein (PID 4376496) was also expressed <SEQ ID 313; cp6496>:

| | |
|---|---|
| 1 | MLIGRYSSDD QFTEATKNTP TIIKLGFVRD NLEGTNPIS EIVSETSSSI |
| 51 | KDSVLRSLPI LGSILGCARL YSTLSTNDPL DETQEKIWHT IFGALETLGL |
| 101 | GILILLFKII FVILHCIFHL VIGFCK* |

The cp6496 nucleotide sequence <SEQ ID 314> is:

| | |
|---|---|
| 1 | ATGCTAATAG GCAGATACAG TAGTGATGAC CAATTCACTG AAGCAACAAA |
| 51 | AAACACCCCA ACCATAATTA AGCTAGGTTT TGTTAGAGAT AATCTCGAGG |
| 101 | GATTAACGAA CCCTATCTCT GAAATCGTCT CGGAAACCTC CTCTTCTATT |
| 151 | AAAGATTCCG TTCTTCGCTC TCTTCCTATT TTAGGGPCCA TTTTAGGATG |
| 201 | CGCCCGACTT TACAGCACAC TCTCTACAAA TGATCCTCTT GACGAAACTC |
| 251 | AAGAAAAGAT TTGGCACACT ATATTTGGAG CCTTAGAAAC CTTAGGCTTA |
| 301 | GGGATTCTCA TCCTCTTATT TAAAATTATT TTTGTTATAT TACACTGCAT |
| 351 | ATTTCATCTA GTTATTGGGT TCTGCAAATA A |

The PSORT algorithm predicts inner membrane (0.5989).

The following C. pneumoniae protein (PID 4376654) was also expressed <SEQ ID 315; cp6654>:

| | |
|---|---|
| 1 | MKTKMNSRKK AGQWAIFNSP TPGVSSTLVL AWTPWGYYDK DVQDILERKD |
| 51 | PMSSSLSEKD SKEFLKNLFV DLLENGFTSV HIHAEEAFTP LDHTGKPHFK |
| 101 | RDNVYLPGKL LGALNEAAVQ ANVSADTQFT LFLTQDECNP FHDKKRG* |

The cp6654 nucleotide sequence <SEQ ID 316> is:

| | |
|---|---|
| 1 | ATGAAAACTA AAATGAACTC TAGAAAAAAA GCAGGTCAAT GGGCAATTTT |
| 51 | CAATTCTCCA ACTCCTGGTG TCAGTTCAAC TTTAGTTTTA GCATGCACTC |
| 101 | CTTGGGGTTA TTACGACAAG GATGTACAAG ATATCTTAGA AAGAAAAGAT |
| 151 | CCGATGAGCT CTTCGCTTTC TGAAAAAGAC TCAAAGGAGT TCTTGAAAAA |
| 201 | TCTGTTTGTA GATCTCTTAG AAAATGGCTT CACATCAGTA CATATTCACG |
| 251 | CAGAAGAAGC TTTCACTCCT CTTGATCATA CCGGGAAACC TCACTTTAAA |
| 301 | AGAGACAATG TGTACTTACC CGGAAAGTTG TTAGGCGCCT TGAATGAGGC |
| 351 | TGCGGTACAA GCCAATGTAA GTGCGGATAC TCAATTTACA TTGTTCCTTA |
| 401 | CTCAAGATGA GTGCAATCCT TTTCATGATA AGAAAAGAGG TTAA |

The PSORT algorithm predicts cytoplasm (0.0730).

The proteins were expressed in E. coli and purified as his-tag products (FIG. 154A; 6412 lanes 2-3; 6431=lanes 11-12; 6443=lanes 5-6; 6496=lanes 8-9; 6654=lane 10; markers in lanes 1, 4, 7). The recombinant proteins were used to immunise mice, whose sera were used in Western blots (FIGS. 154B, 155, 156, 157 & 158) and for FACS analysis.

These experiments show that cp6412, cp6431, cp6443, cp6496 & cp6654 are surface-exposed and immunoaccessible proteins and that they are useful immunogens. These properties are not evident from their sequences alone.

### Example 159 and Example 160

The following C. pneumoniae protein (PID 4376477) was expressed <SEQ ID 317; cp6477>:

| | |
|---|---|
| 1 | LLKFFLVCEE LCILTVATHR ALLETPLALS FFKELKTKYV YRAKDILQLH |
| 51 | NYKGFTILNT SPLCS* |

The cp6477 nucleotide sequence <SEQ ID 318> is:

| | |
|---|---|
| 1 | TTGCTAAAGT TCTTTCTAGT ATGTGAAGAG TTATGTATAC TTACTGTTGC |
| 51 | TACACATAGA GCTCTCTTAG AAACTCCTTT AGCTCTATCA TTTTTTAAAG |
| 101 | AACTTAAGAC AAAATATGTC TACAGGGCGA AAGACATACT ACAACTACAT |
| 151 | AACTATAAAG GATTTACTAT CCTTAATACA TCACCGTTAT GTTCTTAA |

The PSORT algorithm predicts inner membrane (0.128).

The following C. pneumoniae protein (PID 4376435) was also expressed <SEQ ID 319; cp6435>:

| | |
|---|---|
| 1 | LWSHFPRGFF MLPFCPTILL AKPFLNSENY GLERLAATVD |

SYFDLGQSQI

| | |
|---|---|
| 51 | VFLSKQDQGI TVEELSAKDR KPKPGSMNCT LYTEDPILPA |

HNSPSNCSDI

| | |
|---|---|
| 101 | QMRTPISPIH * |

The cp6435 nucleotide sequence <SEQ ID 320> is:

| | |
|---|---|
| 1 | TTGTGGTCGC ATTTCCCAAG AGGATTTTTT ATGCTCCCTT |

TTTGCCCTAC

| | |
|---|---|
| 51 | CATCCTTCTT GCTAAACCTT TTTTAAATAG CGAGAATTAC |

GGCTTAGAAC

| | |
|---|---|
| 101 | GTTTAGCTGC AACCGTAGAT TCTTATTTTG ATCTGGGACA |

GTCTCAAATA

| | |
|---|---|
| 151 | GTCTTCCTAA GCAAACAGGA TCAAGGAATC ACTGTGGAAG |

AATTGAGTGC

| | |
|---|---|
| 201 | TAAAGATAGG AAATTCAAGC CAGGCTCTAT GAACTGTACA |

CTGTACACTG

| | |
|---|---|
| 251 | AAGATCCTAT CTTACCTGCT CATAATTCCT TTAGTAATTG |

CTCTGATATT

| | |
|---|---|
| 301 | CAAATGCGTA CTCCGATTAG CCCTATACAT TAA |

The PSORT algorithm predicts periplasmic space (0.4044).

The proteins were expressed in E. coli and purified as his-tag products (FIG. 159A; 6435 lanes 2-4; 6477=lanes 5-7). The recombinant proteins were used to immunise mice, whose sera were used in Western blots (FIGS. 159B & 160) and for FACS analysis.

These experiments show that cp6477 & cp6435 are surface-exposed and immunoaccessible proteins and that they are useful immunogens. These properties are not evident from the sequences alone.

### Example 161 and Example 162 and Example 163

The following C. pneumoniae protein (PID 4376441) was expressed <SEQ ID 321; cp6441>:

| | |
|---|---|
| 1 | VEAGANVLVI DTAHAHSKGV FQTVLEIKSQ FPQKSLVVGN |

LVTAEAAVSL

| | |
|---|---|
| 51 | AEIGVDAVKV GIGPGSICTT RIVSGVGYPQ ITAITNVAKA |

LKNSAVTVIA

| | |
|---|---|
| 101 | DGRIRYSGDV VKALAAGADC VMLGSLLAGT DEAPGDIVSI |

DEKLFKRYRG

| | |
|---|---|
| 151 | MGSLGAMKQG SADRYFQTQG QKRLVPGGVE QLVAYKGSVH |

DVLYQILGGI

| | |
|---|---|
| 201 | RSGMGYVGAE TLKDLKTKAS PVRKTESGRA ESHIHNIYKV |

QPTLNY

The cp6441 nucleotide sequence <SEQ ID 322> is:

| | |
|---|---|
| 1 | GTGGAAGCTG GAGCAAATGT TCTAGTCATT GACACAGCTC |

ATGCACACTC

| | |
|---|---|
| 51 | TAAAQGAGTA TTCCAAACAG TTTTAGAAAT AAAATCCCAG |

TTCCCACAAA

| | |
|---|---|
| 101 | TTTCTTTAGT TGTAGGGAAT CTTGTTACAG CTGAAGCCGC |

AGTTTCCTTA

| | |
|---|---|
| 151 | GCTGAGATTG GAGTTGACGC TGTAAAGGTA GGTATTGGCC |

CAGGATCTAT

| | |
|---|---|
| 201 | CTGTACAACT AGAATCGTTT CAGGGGTCGG TTATCCACAA |

ATTACTGCCA

| | |
|---|---|
| 251 | TTACAAACGT AGCAAAAGCT CTTAAAAACT CTGCCGTGAC |

TGTAATTGCT

| | |
|---|---|
| 301 | GATGGGAGAA TCCGCTATTC TGGAGATGTG GTAAAAGCAT |

TAGCAGCACG

| | |
|---|---|
| 351 | AGCAGACTGT GTCATGCTAG GAAGTTTGCT TGCAGGGACT |

GATGAAGCTC

| | |
|---|---|
| 401 | CTGGGGATAT CGTTTCTATC GATGAGAAGC TTTTTAAAAG |

GTACCGCGGC

| | |
|---|---|
| 451 | ATGGGATCTT TAGGCGCTAT GAAACAAGGA AGTGCTGACC |

GGTATTTTCA

| | |
|---|---|
| 501 | AACACAGGGA CAGAAAAAGC TGGTTCCTGG GGGAGTTGAA |

GGACCGCTCG

| | |
|---|---|
| 551 | CTTATAAAGG CTCTGTCCAC GATGTCCTCT ATCAAATTTT |

AGGAGGAATA

| | |
|---|---|
| 601 | CGCTCAGGTA TGGGGTATGT TGGAGCTGAA ACTCTCAAAG |

ATTTAAAAAC

| | |
|---|---|
| 651 | TAAGGCTTCC TTTGTTCGAA TTACTGAATC TGGAAGAGCT |

GAAAGTCATA

| | |
|---|---|
| 701 | TTCATAATAT TTACAAAGTT CAACCAACCT TAAATTATTA |

A

The PSORT algorithm predicts bacterial inner membrane (0.132).

The following C. pneumoniae protein (PID 4376748) was also expressed <SEQ ID 323; cp6748>:

| | |
|---|---|
| 1 | LFSEGTALNL FRIFAPLRNR VTTEYSRARQ PDLHRIAIVY |

IGVLDSESSK

| | |
|---|---|
| 51 | ILERLISYMS CIYSESQMYL RFFMGKNVNQ SAVLSKLHVE |

NLHIRCGFFS

| | |
|---|---|
| 101 | EDAVPESEPF DLSIYVHTDR SCPLPTKKRS SSWELQTVEL |

PESIYPQSEF

| | |
|---|---|
| 151 | LLMRORMLS* |

The cp6748 nucleotide sequence <SEQ ID 324> is:

| | |
|---|---|
| 1 | TTGTTCTCTG AGGGGACAGC TCTAAATTTA TTTCGTATAT |

TTGCTCCACT

| | |
|---|---|
| 51 | ACGCAACCGT GTGACThCAG AATACAGTCG TGCTAGGCAA |

CCCGACCTAC

| | |
|---|---|
| 101 | ATAGAATTGC CATCGTCTAT ATAGGAGTTC TCGATTCAGA |

AAGTTCCAAG

| | |
|---|---|
| 151 | ATCCTAGAGC GGCTAATCTC TTATATGAGT TGTATCTATT |

CTGAATCGCA

| | |
|---|---|
| 201 | AATGTATTTA AGATTCTTTA TGGGCAAGAA TGTAAATCAA |

AGTGCTGTAC

| | |
|---|---|
| 251 | TCTCAAAATT ACATGTAGAA AATCTGCACA TCCGTTGTGG |

CTTTTTCAGC

| | |
|---|---|
| 301 | GAGGATGCTG TTCCAGAGAG TGAGCCCTTC GATCTCTCCA |

TCTACGTGCA

| | |
|---|---|
| 351 | CACAGATCGT AGCTGTCCTC TCCCTACGAA AAAACGGAGC |

AGCTCCTGGG

| | |
|---|---|
| 401 | AACTCCAAAC TGTAGAACTC CCAGAGTCAA TATATCCACA |

GTCGGAATTC

| | |
|---|---|
| 451 | CTATTGATGA GACCTCGAAT GCTTTCGTAG |

The PSORT algorithm predicts cytoplasm (0.170).

The following C. pneumoniae protein (PID 4376881) was also expressed <SEQ ID 325; cp6881>:

| | |
|---|---|
| 1 | MRPHRKHVSS KSLALKQSAS THVEITTKAF RLSMPLKQLI |

LEKSDHLPPM

| | |
|---|---|
| 51 | ETIRVVLTSH KDKLGTBVHV VASHGKEILQ TKVHNANPYT |

AVINAFKKIR

| | |
|---|---|
| 101 | TMANKHSNKR KDRTKHDLGL AAKEERIAIQ EEQEDRLSNE |

WLPVEGLDAW

| | |
|---|---|
| 151 | DSLKTLGYVP ASAKKKISKK KMSIRMLSQD EAIRQLESAA |

ENFLIFLNEQ

| | |
|---|---|
| 201 | EHRIQCIYKR HDGNYVLIEP SLKPGFCI* |

The cp6881 nucleotide sequence <SEQ ID 326> is:

| | |
|---|---|
| 1 | ATGAGACCTC ATCGTAAACA CGTATCATCT AAAAGCTTAG |

CTTTAAAGCA

| | |
|---|---|
| 51 | ATCTGCATCA ACTCATGTAG AGATCACAAC AAAAGCCTTT |

CGTCTCTCTA

| | |
|---|---|
| 101 | TGCCTCTAAA ACAGCTGATC CTAGAGAAAA GCGACCACCT |

CCCCCCTATG

| | |
|---|---|
| 151 | GAAACAATCC GTGTGGTGCT AACCTCTCAT AAAGATAAGC |

TAGGCACCGA

| | |
|---|---|
| 201 | GGTGCATGTT GTAGCTTCTC ATGGCAAAGA AATCCTTCAA |

ACTAAGCTTC

| | |
|---|---|
| 251 | ATAACGCAAA CCCATACACT GCAGTGATCA ATGCTTTTAA |

GAAAATCCGC

| | |
|---|---|
| 301 | ACCATGGCAA ATAAGCACTC CAATAAACGT AAAGACAGGA |

CAAAACATGA

| | |
|---|---|
| 351 | TCTAGGTCTT GCAGCAAAAG AAGAACGTAT CGCAATACAG |

GAAGAACAAG

| | |
|---|---|
| 401 | AAGATCGCCT TAGCAACGAG TGGCTTCCTG TCGAAGGCCT |

CGATGCCTGG

| | |
|---|---|
| 451 | GATTCTCTAA AAACTCTTGG GTATGTTCCC GCATCAGCGA |

AAAAGAAGAT

| | |
|---|---|
| 501 | CTCCAAGAAA AAGATGAGCA TTCGTATGCT ATCTCAAGAC |

GAGGCTATCC

| | |
|---|---|
| 551 | GCCAGCTAGA GTCTGCCGCA GAAAACTTCC TGATCTTCTT |

GAACGAGCAA

| | |
|---|---|
| 601 | GAGCATAAAA TCCAATGCAT TTATAAAAAA CATGACGGCA |

ACTATGTCCT

| | |
|---|---|
| 651 | TATTGAACCT TCCCTCAAGC CAGGATTCTG CATCTGA |

The PSORT algorithm predicts cytoplasm (0.249).

The proteins were expressed in E. coli and purified as his-tag products (FIG. 161A; 6441=lanes 7-9; 6748=lanes 2-3; 6881=lanes 4-6). The recombinant protein was used to immunise mice, whose sera were used in Western blots (FIGS. 161B, 162 & 163) and for FACS analysis.

These experiments show that cp6441, cp6748 & cp6881 are surface-exposed and immunoaccessible proteins and that they are useful immunogens. These properties are not evident from the sequence alone.

### Example 164 and Example 165 Example 166

The following C. pneumoniae protein (PID 4376444) was expressed <SEQ ID 327; cp6444>:

| | |
|---|---|
| 1 | MEQPNCVKQD TTTVLYALNS FDPRLSDDTH RLGKQSPLEA |

ENALGEFIEG

| | |
|---|---|
| 51 | LDTNSFPLEE VAIPILPGYH PKFYLSFIDR DDQGVHYEVL |

DGVFLKTVAA

| | |
|---|---|
| 101 | CIIENSFLTD SHSPELLSEV KEALKR* |

The cp6444 nucleotide sequence <SEQ ID 328> is:

| | |
|---|---|
| 1 | ATGGAGCAAC CCAATTGTGT GATTCAGGAT ACTACAACTG |

TTTTGTATGC

| | |
|---|---|
| 51 | CTTAAATAGC TTTGATCCTA GACTTAGTQA TGACACTCAC |

AGACTTGGGA

| | |
|---|---|
| 101 | AGCAATCACC TCTTGAAGCA GAAAATGCTC TTGGAGAATT |

TATTGAAGGT

| | |
|---|---|
| 151 | TTGGATACAA ATAGCTTTCC TTTAGAGGAA GTTGCCATTC |

CCATCCTGCC

| | |
|---|---|
| 201 | AGGTTATCAC CCTAAGTTTT ATTTATCTTT CATAGATAGG |

GACGATCAAG

| | |
|---|---|
| 251 | GTGTCCACTA TGAAGTTTTA GATGGCGTAT TTTTAAAGAC |

AGTCGCTGCT

| | |
|---|---|
| 301 | TGTATTATAG AGAACTCCTT CTTAACTGAT TCTATGAGCC |

CGGAGCTTCT

| | |
|---|---|
| 351 | CAGCGAAGTT AAGGAAGCTC TGAAACGATG A |

The PSORT algorithm predicts cytoplasm (0.2031).

The following C. pneumoniae protein (PID 4376413) was also expressed <SEQ ID 329; cp6413>:

| | |
|---|---|
| 1 | MAVQSIKEAV TSAATSVGCV NCSREAIPAF NTEERATSIA |

RSVIAAIIAV

| | |
|---|---|
| 51 | VAISLLGLGL VVLAGCCPLG MAAGAITMLL GVALLAWAIL |

ITLRLLNIPK

| | |
|---|---|
| 101 | ABIPSPGNNG EPNBRNSATP PLBGGVAGEA GRGGGSPLTQ |

LDLNSGAGS*

The cp6413 nucleotide sequence <SEQ ID 330> is:

| | |
|---|---|
| 1 | ATGGCTGTTC AATCTATAAA AGAAGCCGTA ACATCAGCCG |

CAACATCAGT

| | |
|---|---|
| 51 | AGGATGTGTA AACTGTTCTA GAGAGGCTAT ACCAGCATTT |

AATACAGAGG

| | |
|---|---|
| 101 | AGAGAGCAAC GAGTATTGCT AGATCTGTTA TAGCAGCTAT |

CATTGCTGTT

| | |
|---|---|
| 151 | GTAGCTATCT CCTTACTCGG ACTAGGTCTT GTAGTTCTTG |

CTGGTTGCTG

| | |
|---|---|
| 201 | TCCTTPAGGA ATGGCTGCGG GTGCTATAAC AATCCTGCTG |

GGTGTAGCAT

| | |
|---|---|
| 251 | TATTAGCTTG GGCAATACTG ATTACTTTGA GACTGCTTAA |

TATACCTAAG

| | |
|---|---|
| 301 | GCTGAAATAC CGAGTCCAGG GAACAACGGT GAGCCTAATG |

AAAGAAATTC

| | |
|---|---|
| 351 | AGCAACTCCT CCTCTAGAGG GTGGTGTTGC AGGAGAAGCC |

GGTCGCGGCG

| | |
|---|---|
| 401 | GGGGGTCACC TTTAACCCAA CTTGATCTCA ATTCAGGGGC |

GGGAAGTTAG

The PSORT algorithm predicts inner membrane (0.6180).

The following C. pneumoniae protein (PID 4377391) was also expressed <SEQ ID 331; cp7391>:

| | |
|---|---|
| 1 | NNLRVIELPL LPIKQALEKA PVQYNSYKAR LTKVEPCFRE |

SPAYITSEER

| | |
|---|---|
| 51 | LQSLDQTLER AYKEYQKRFQ EPSRLESEVS GCREHLREQV |

KQFETQGLDL

| | |
|---|---|
| 101 | IKGELIFVSD VLFRKMVSCL VSTVHVPFMB FYYEYFELHR |

LRLRAQWMAN

| | |
|---|---|
| 151 | AEIYSKVRKA FPEMLKETLE KAKAPREEGY WLLCEEGRSR |

EKRLILNKIE

| | |
|---|---|
| 201 | AAQQRVKDLE PPPIKETGKQ REKKEYSFFI RLKS* |

The cp7391 nucleotide sequence <SEQ ID 332> is:

| | |
|---|---|
| 1 | ATGATGCTTC GTGTCATAGA GCTTCCACTA CTTCCTATAA |

AGCAAGCGTT

| | |
|---|---|
| 51 | GGAGAAGGCT TTTGTACAAT ATAATAQCTA CAAAGCGAAG |

TTAACCAAGG

| | |
|---|---|
| 101 | TAGAACCTTG CTTTAGAGAG AGCCCTGCCT ATATAACTAG |

CGAAGAGCGA

| | |
|---|---|
| 151 | CTCCAGAGTT TGGATCAGAC TTTAGAACGT GCGTACAAAQ |

AGTACCAGAA

| | |
|---|---|
| 201 | GAGATTCCAG GAGCCTTCAC GTTTGGAATC GGAAGTAAGT |

GGATGTAGAG

| | |
|---|---|
| 251 | AGCATCTTAG AGAGCAGGTA AAACAATTTG AAACTCAAGG |

ACTAGACTTG

| | |
|---|---|
| 301 | ATCAAAGAAG AGCTTATTTT TGTTAGTGAT GTGTGATTCC |

GAAAAGTGGT

| | |
|---|---|
| 351 | CAGTTGTCTA GTGTCGACAG TGCATGTTCC CTTTATGGAG |

TTTTATTATG

| | |
|---|---|
| 401 | AGTATTTTGA GTTGCATAGA TTGAGGTTGC GGGCCCAATG |

GATGGCGAGT

| | |
|---|---|
| 451 | GCCGAGATTT ATAGCAAAGT TAGAAAAGCA TTCCCAGAGA |

TGTTGAAGGA

| | |
|---|---|
| 501 | GACCTTAGAA AAAGCTAAGG CTCCCAGAGA AGAAGAGTAT |

TGGTTACTTT

| | |
|---|---|
| 551 | GCGAGGAGAC AAAGAGTAAG GAGAAGCGTT TGATTCTCAA |

CAAGATAGAG

| | |
|---|---|
| 601 | GCAGCTCAGC AGCGGGTAAA AGATTTAGAA CCTCCTCCTA |

TTAAAGAGAC

| | |
|---|---|
| 651 | AGGGAAACAG AAACGGAAGA AAGAATATTC GTTTTTCATT |

CGATTAAAAT

| | |
|---|---|
| 701 | CGTGA |

The PSORT algorithm predicts inner membrane (0.1489).

The proteins were expressed inE. coli and purified as his-tag and GST-fusion products (FIG. 164A; 6444-lanes 11-12; 7391=lanes 2-3; 6413=lanes 4-6). The recombinant protein was used to immunise mice, whose sera were used in Western blots (FIGS. 164B, 165 & 166) and for FACS analysis.

These experiments show that cp6444, cp6413 & cp7391 are surface-exposed and immunoaccessible proteins and that they are useful immunogens. These properties are not evident from the sequence alone.

### Example 167, Example 168, Example 169 and Example 170

The following C. pneumoniae protein (PID 4376463) was expressed <SEQ ID 333; cp6463>:

| | |
|---|---|
| 1 | MKKKVTIDEA LKEILRLEGA ATQEELCAKL LAQGFATTQS |

SVSRWLRKIQ

| | |
|---|---|
| 51 | AVKVAGERGA RYSLPSSTEK TTTRHLVLSI RHNASLIVIR |

TVPGSASWIA

| | |
|---|---|
| 101 | ALLDGGLKDE ILGTLAGDDT IFVTPIDEGR LPLLMVSIAN |

LLGVPLD*

The cp6463 nucleotide sequence <SEQ ID 334> is:

| | |
|---|---|
| 1 | ATGAAAAAAA AAGTAACTAT AGATGAGGCT TTAAAAGAAA |

TTTTACGTCT

| | |
|---|---|
| 51 | TGAAGGAGCG GCAACTCAGG AGGAATTATG TGCAAAACTC |

TTAGCTCAAG

| | |
|---|---|
| 101 | GTTTTGCTAC AACCCAGTCG TCTGTATCTC GTTGGCTACG |

AAAGATTCAG

| | |
|---|---|
| 151 | GCTGTAAAGG TTGCTGGAGA GCGTGGTGCT CGTTATTCTT |

TACCCTCTTC

| | |
|---|---|
| 201 | AACAGAGAAG ACCACGACCC GTCATTTGGT GCTCTCTATT |

CGCCATAACG

| | |
|---|---|
| 251 | CCTCTCTTAT TGTAATTCGT ACGGTTCCTG GTTCAGCTTC |

TTGGATCGCT

| | |
|---|---|
| 301 | GCTTTGTTAG ATCAAGGGCT CAAAGATGAA ATTCTTGGAA |

CTTTGGCAGG

| | |
|---|---|
| 351 | AGATGACACG ATTTTTGTCA CTCCTATAGA TGAAGGGAGG |

CTCCCATTGT

| | |
|---|---|
| 401 | TGATGGTTTC GATTGCAAAT TTACTGCAAG TTTTCTTGGA |

TTAA

The PSORT algorithm predicts inner membrane (0.1510).

The following C. pneumoniae protein (PID 4376540) was also expressed <SEQ ID 335; cp6540>:

| | |
|---|---|
| 1 | MSGCGSSSTS TWEWMKSFVP NWRNPTPPLS PIPSEDRFIL |

AYEPFVLPKT

| | |
|---|---|
| 51 | DPENAGGNPP GTSTPNVENG IDDLNPLLGG PNEGNNANNP |

GTSGSNPTSL

| | |
|---|---|
| 101 | PAPERLPGTE ENSGEEEGGS GNNEDLIG* |

The cp6540 nucleotide sequence <SEQ ID 336> is:

| | |
|---|---|
| 1 | ATGTCTCAAT GTCAGAGTAG CAGTACATCT ACCTGGGAAT |

GGATGAAATC

| | |
|---|---|
| 51 | TTTTGTGCCA AACTGGAAGA ATCCAACTCC CCCCTTATCT |

CCTATACCTT

| | |
|---|---|
| 101 | CTGAGGACGA ATTTATATTA GCATACGAGC CATTTGTTCT |

ACCGAAAACA

| | |
|---|---|
| 151 | GATCCAGAAA ACGCACAAGC TAATCCTCCA GGCACATCTA |

CACCGAATGT

| | |
|---|---|
| 201 | AGAAAACGGG ATCGATGATC TCAACCCTCT TCTGGGGCAA |

CCCAACGAAC

| | |
|---|---|
| 251 | AAAACAATGC CAACAATCCA GGAACTTCTG GATCTAATCC |

TACATCTCTA

| | |
|---|---|
| 301 | CCCGCCCCCG AACGACTCCC TGAAACTGAA GAGAACAGCC |

AAGAAGAAGA

| | |
|---|---|
| 351 | ACAAGGATCT CAAAATAATG AGGATCGGAT AGGATAA |

The PSORT algorithm predicts cytoplasm (0.3086).

The following C. pneumoniae protein (PID 4376743) was also expressed <SEQ ID 337; cp6743>:

| | |
|---|---|
| 1 | LREEGSVSFR EYPRAYMCDK IVAGKNFLFT LDAVIKGAGW |

RSGEKLNLFY

| | |
|---|---|
| 51 | VESGALGREI KVSLEEYIQS MVGILGSGRT KKSFKFSVDP |

TPLGGALGER

| | |
|---|---|
| 101 | CSSDDDGDAT ATSTATGGTA SPTDMHEDE* |

The cp6743 nucleotide sequence <SEQ ID 338> is:

| | |
|---|---|
| 1 | TTGAGAGAAG AAGGTAGTGT TTCTTTCAGA GAATATTTCA |

GAGCCTATAT

| | |
|---|---|
| 51 | GTGTGATAAA ATCGTGGCAC ACAAGAACTT CTTATGTACT |

TTAGACGCTG

| | |
|---|---|
| 101 | TAATTAAACA GGCCGGTTGG AGATCACAAG AGAAACTCAA |

TTTATTTTAT

| | |
|---|---|
| 151 | GTTGAAAGTC AGGCTTTAGG AAGAGAAATC AAAGTCAGCT |

TAGAGGAATA

| | |
|---|---|
| 201 | TATTCAGAGT ATGGTCGGGA TTTTGGGATC TCAGAGAACC |

AAGAAAAGCT

| | |
|---|---|
| 251 | TTAAGTTTTC TGTCGACTTT ACCCCTTTAG AGCAGGCTCT |

ACAAGAAAGA

| | |
|---|---|
| 301 | TGCTCTTCTG ATGATGACGA AGATGCAACA GCAACTTCGA |

CCGCTACAGG

| | |
|---|---|
| 351 | GGCAACAGCA TCTCCGACTG ACATGCACGA AGATGAGTAA |

The PSORT algorithm predicts cytoplasm (0.2769).

The following C. pneumoniae protein (PID 4377041) was also expressed <SEQ ID 339; cp7041>:

| | |
|---|---|
| 1 | MLMMLMMIIG ITGGSGAGKT TLTQNIKEIF GEDVSVICQD NYYKDRSHYT |
| 51 | PEERANLIWD HPDAFNDDLL ISDIKRLKNN EIVQAPVFDF VLGNRSKTEI |
| 101 | ETIYPSXVIL VEGILVFENQ ELRDLMDIRI FVDTDADERI LRRMVRDVQE |
| 151 | QGDSVDCIMS RYLSMVKPMH EKFIEPTRKY ADIIVHGNYR QNVVTNILSQ |
| 201 | KIKNHLENAL ESDETYYMVN SK* |

The cp7041 nucleotide sequence <SEQ ID 340> is:

| | |
|---|---|
| 1 | ATGTTGATGA TGCTTATGAT GATTATTGGA ATTACAGOAG GTTCTGGAGC |
| 51 | TGGGAAAACC ACCCTAACCC AAAACATTAA AGAAATTTTC GGTGAGGATG |
| 101 | TGAGTGTTAT CTGCCAAGAT AATTATTACA AAGATAGAPC TCATTATACT |
| 151 | CCTGAAGAAC GTGCCAATTT AATTTGGGAT CATCCGGACG CCTTTGATAA |
| 201 | TGACTTATTA ATTTCAGACA TAAAACGTCT AAAAAATAAT GAGATTGTCC |
| 251 | AAGCCCCAGT TTTTGATTTT GTPTTAGGTA ATCGATCTAA AACGGAGATA |
| 301 | GAAACGATCT ATCCATCTAA AGTTATTCTT GTTGAAGGTA TTCTGGTCTT |
| 351 | TGAAAATCAA GAACTTAGAG ATCTTATGGA TATTAGGATC TTTGTAGACA |
| 401 | CCGATGCTGA TGAAAGGATA CTACGCCGTA TGGTTCGAGA TGTTCAAGAA |
| 451 | CAAGGAGATA GCGTGGACTG CATCATGTCT CGTTATCTTT CTATGGTAAA |
| 501 | GCCTATGCAT GAGAAATTTA TAGAGCCGAC TCGGAAATAT GCTGATATCA |
| 551 | TTGTACATGG AAATTACCGA CAAAACGTAG TAACAAATAT TTTGTCACAG |
| 601 | AAAATTAAAA ATCATTTAGA GAATGCCCTG GAAAGCGATG AGACGTATTA |
| 651 | TATGGTCAAC TCTAAGTAA |

The PSORT algorithm predicts inner membrane (0.1022).

The proteins were expressed in E. coli and purified as his-tag products (FIG. 167A; 6463 lanes 2-4; 6540=lanes 5-7; 6743=lanes 8-9; 7041=lanes 10-11). The recombinant proteins were used to immunise mice, whose sera were used in Western blots (FIGS. 167B, 168, 169 & 170) and for FACS analysis.

These experiments show that cp6463, cp6540, cp6743 & cp7041 are surface-exposed and immunoaccessible proteins and that they are useful immunogens. These properties are not evident from the sequence alone.

### Example 171 and Example 172 and Example 173

The following C. pneumoniae protein (PID 4376632) was expressed <SEQ ID 341; cp6632>:

| | |
|---|---|
| 1 | VQLFQYNNES GWDWLCDFPS QGEGFQLSRL VGLLHSSWAL YEAKEQFYLP |
| 51 | EVSLLTWEEL IEMQLLSKPT KHGVAKDLCN VPHKHFQRFR QYLGSLDLNQ |
| 101 | RFENTFLNYP KYHLDRE* |

The cp6632 nucleotide sequence <SEQ ID 342> is:

| | |
|---|---|
| 1 | GTGCAATTAT TTCAATATAT GAATGAGTCC GGATGGGATT GGCTTTGTGA |
| 51 | TTTTGATTCT CAAGGCGAGG GATTCCAGTT ATCACGTCTG GTTGGGCTGT |
| 101 | TACATTCGTC CTGGGCATTA TACGAAGCAA AAGAGCAATT TTACCTTCCT |
| 151 | GAGGTTTCTC TATTGACCTG GGAAGAACTG ATAGAAATGC AGTTATTAAG |
| 201 | CAAACCAACA AAACACGGGG TTGCAAAAGA TCTTTGTAAT GTATTTGAAA |
| 251 | AACACTTTCA AAGGTTTAGA CAGTACCTAG GTTCCTTAGA TCTAAATCAA |
| 301 | AGGTTCGAAA ATACCTTCTT GAATTATCCT AAATACCATT TAGATAGGGA |
| 351 | GTGA |

The PSORT algorithm predicts cytoplasm (0.3627).

The following C. pneumoniae protein (PID 4376648) was also expressed <SEQ ID 343; cp6648>:

| | |
|---|---|
| 1 | MPVSSAPLPT SHRPSSGNLG LMEPNSKALK AKHQDKTTKT IKLLVEILVA |
| 51 | ILVIEVLGII AAFFIPGTPP ICLIILGGLI LTPVLCVLLL VIKLALVNKT |
| 101 | EGTTAEQQIK RKLSSKSIS* |

The cp6648 nucleotide sequence <SEQ ID 344> is:

| | |
|---|---|
| 1 | ATGCCCGTGT CCTCAGCCCC CCTACCCACA AGCCACCGCC CTTCCTCTGG |
| 51 | AAATCTACGC CTCATGGAAC CAAATTCCAA AGCTCTAAAA GCAAAGCATC |
| 101 | AAGATAAAAC GACGAAGACG ATTAAACTTT TAGTTAAAAT CCTTGTTGCC |
| 151 | ATTCTAGTAA TAGAAGTTTT AGGAAThATT GCAGCTTTCT TTATTCCTGG |
| 201 | GACTCCTCCC ATCTGCTTGA TTATCCTAGG AGGCCTTATT CTTACAACAG |
| 251 | TACTCTGTGT GCTTCTTCTT GTTATAAAGC TTGCCCTTGT AAACAAAACC |
| 301 | GAAGGAACAA CTGCTGAACA GCAGATAAAA CGTAAACTCT CTTCTAAAAG |
| 351 | TATTTCTTAG |

The PSORT algorithm predicts inner membrane (0.6074).

The following C. pneumoniae protein (PID 4376497) was also expressed <SEQ ID 345; cp6497>:

| | |
|---|---|
| 1 | MRPNSIIFLE NTKHYPDIFR EGEVRDRMGL NEASDWLLST EITIIRSILG |
| 51 | AIPILGNILG AGRLYSVWYT SDEDWRKQVV * |

The cp6497 nucleotide sequence <SEQ ID 346> is:

| | |
|---|---|
| 1 | ATGAAGCCAA ATAGTATTAT TTTTTTAGAA AATACTAAGC ATTATCCCGA |
| 51 | CATCTTTCGA GAAGGATTTG TTCGTGATCG TCATGGACTA ATGGAAGCCT |
| 101 | CGGATTGGTT ACTTTCTACG GAAATTACGA TCATTCGCTC CATTCTGGGA |
| 151 | GCTATCCCTA TTTTAGGAAA TATTCTTGGA GCCGGACGAC TCTATAGCGT |
| 201 | TTGGTATACA AGTGACGAAG ATTGGAAAAA ACAAGTGGTT TGA |

The PSORT algorithm predicts inner membrane (0.145).

The proteins were expressed in E. coli and purified as his-tag products (FIG. 171A; 6632=lanes 5-7; 6648=lanes 8-10; 6497=lanes 2-4). The recombinant proteins were used to immunise mice, whose sera were used in Western blots (FIGS. 171B, 172, 173) and for FACS analysis.

These experiments show that cp6632, cp6648 and cp6497 are surface-exposed and immunoaccessible proteins and that they are useful immunogens. These properties are not evident from the sequence alone.

### Example 174, Example 175, Example 176, Example 177 and Example 178

The following C. pneumoniae protein (PID 4377200) was expressed <SEQ ID 347; cp7200>:

| | |
|---|---|
| 1 | MPVPIDNSSR NLQEVPESLE DLBQHAEESP THQSAESSSL QLSLASSAIS |
| 51 | SRVEQLSSLV LGNENSDPSS LRDVPIFSAI YESSTHTPVP TPLVGVGYIN |
| 101 | GSQSGYYDTQ RBSLHLSQLL GSRRVEVVYN QGNFNEASLL NLCPRRPRRD |
| 151 | PSPISLALLE LWEAPFLEHP PGSTFNPIFP W* |

The cp7200 nucleotide sequence <SEQ ID 348> is:

| | |
|---|---|
| 1 | ATGCCCGTTC CTATAGATAA TTCCTCTCGC AACCTACAAG AAGTTCCAGA |
| 51 | AAGCCTAGAA GACCTCGAAC AACACGCAGA AGAATCTCCT ACTCATCAAA |
| 101 | GTGCAGAAAG CAGTTCTTTG CAACTGTCTC TAGCCTCCTC AGCAATTTCT |
| 151 | AGTAGAGTAG AACAACTATC TTCCCTCGTC TTAGGAATGG AAAATTCAGA |
| 201 | TTTCTCCTCT TTAAGAGACG TTCCTATCTT CTCAGCTATC TACGAATCTT |
| 251 | CAACACACAC ACCTGTCCCC ACTCCTCTAG TTGGCGTGGG ATATATCAAC |
| 301 | GGAAGTCAAT CAGGATACTA CGATACACAA AGAGAATCTC TTCACCTCAG |
| 351 | CCAATTGTTA GGAAGCCGAA GAGTTGAAGT TGTCTATAAC CAAGGAAACT |
| 401 | TCATGGACGC CTCTTTGCTA AATCTGTGCC CCAGAAGACC TCGAAGAGAT |
| 451 | CCCTCTCCAA TTTCTTTAGC TCTATTAGAG CTCTGGGAAG CATTTTTTTT |
| 501 | AGAACACCCC CCAGGTAGCA CTTTTAATCC AATATTTTTT TGGTAA |

The PSORT algorithm predicts cytoplasm (0.3672).

The following C. pneumoniae protein (PID 4377235) was also expressed <SEQ ID 349; cp7235>:

| | |
|---|---|
| 1 | LNFVSTLTQS DFYAPVLEKL EEAFADTTGQ VILFSSSPDF |

IVHPIAQQLG

| | |
|---|---|
| 51 | ISSWYASCYR DQSAEQTIYK KCLTGDKKAQ ILSYIKKINQ |

ARSHTFSDHI

| | |
|---|---|
| 101 | LDLPFLMLGE EKTVVRPQGR LKKMAKKYYW NIV* |

The cp7235 nucleotide sequence <SEQ ID 350> is:

| | |
|---|---|
| 1 | TTGAATTTTG TATCGACTCT GACCGGCTCC GATTTTTATG |

CTCCTGTTTT

| | |
|---|---|
| 51 | AGAAAAACTA GAAGAAGCTT TTGCAGATAC CACAGGACAG |

GTGATCCTTT

| | |
|---|---|
| 101 | TTTCTTCTTC TCCAGACTTT ATTGTCCACC CCATAGCGCA |

GCAACTCGGG

| | |
|---|---|
| 151 | ATTAGTTCTT GGTATGCGTC GTGTTATCGC GATCAGTCTG |

CAGAACAGAC

| | |
|---|---|
| 201 | GATCTATAAA AAATGTCTTA CAGGGGATAA AAAAGCGCAA |

ATTTTGAGTT

| | |
|---|---|
| 251 | ATATTAAAAA AATTAATCAA GCAAGAAGCC ATACCTTCTC |

CGACCATATT

| | |
|---|---|
| 301 | TTAGATCTTC CTTTTCTTAT GCTGGGAGAA GAGAAAACCG |

TCGTTCGCCC

| | |
|---|---|
| 351 | TCAGGGACGA CTCAAGAAAA TGGCAAAAAA ATATTACTGG |

AATATCGTTT

| | |
|---|---|
| 401 | AA |

The PSORT algorithm predicts cytoplasm (0.3214).

The following C. pneumoniae protein (PID 4377268) was also expressed <SEQ ID 351; cp7268>:

| | |
|---|---|
| 1 | MMHRYFIPLL ALLIFSPSLV RAELQPSENR KGGWPTQLSC |

AEGSQLFCKF

| | |
|---|---|
| 51 | EAAYNNAIEE GKPGILVFFS ERPTPEFADL TNGSFSLSTP |

IAKGFNVVVL

| | |
|---|---|
| 101 | CPGLISPLDF FHKMDPVILY HGSFLEMFPE VEAVSGPRLC |

YILIDEQGGA

| | |
|---|---|
| 151 | QCQAVLPLET KN* |

The cp7268 nucleotide sequence <SEQ ID 352> is:

| | |
|---|---|
| 1 | ATGATGCACC GTTATTTTAT TCCTTTATTA GCACTTCTCA |

TTTTCTCTCC

| | |
|---|---|
| 51 | TTCTTTAGTC AGGGCAGAGC TACAACCAAG TGAAAACAGA |

AAAGGGGGGT

| | |
|---|---|
| 101 | GGCCTACACA ACTTTCCTGT GCAGAAGGTT CGCAACTCTT |

CTGTAAATTC

| | |
|---|---|
| 151 | GAAGCTGCCT ATAATAATGC AATTGAGGAA GGGAAACCTG |

GGATTTTAGT

| | |
|---|---|
| 201 | CTTTTTCTCT GAGCGACCCA CACCAGAATT TGCCGACTTA |

ACGAATGGTT

| | |
|---|---|
| 251 | CATTTTCTCT CTCTACGCCA ATCGCCAAGG GCTTTAATGT |

CGTTGTGTTA

| | |
|---|---|
| 301 | TGCCCCGGGC TTATCAGTCC CTTAGACTTT TTCCACAAAA |

TGGATCCTGT

| | |
|---|---|
| 351 | GATTCTCTAT ATGGGAAGTT TTCTAGAGAT GTTCCCTGAA |

GTGGAGGCAG

| | |
|---|---|
| 401 | TTAGTGGCCC TCGCTTATGT TATATCTTAA TAGATGAACA |

GGGTGGGGCT

| | |
|---|---|
| 451 | CAATGTCAGG CTGTCCTGCC TTTAGAAACA AAGAATTAG |

The PSORT algorithm predicts inner membrane (0.1235).

The following C. pneumoniae protein (PID 4377375) was also expressed <SEQ ID 353; cp7375>:

| | |
|---|---|
| 1 | MQRIIIVGID TGVGKTIVSA ILARALNAEY WKPIQAGNLE |

NSDSNIVHEL

| | |
|---|---|
| 51 | SGAYCHPEAY RLHKPLSPHK AAQIDNVSIE ESHICAPKTT |

SNLIIETSGG

| | |
|---|---|
| 101 | FLSPCTSKRL QGDVFSSWSC SWILVSQAYL GSINHTCLTV |

EAMRSRNLNI

| | |
|---|---|
| 151 | LGMVVNGYPE DEEHWLTQEI KLPIIGTLAK EKEITKTIIS |

CYAEQWKWVW

| | |
|---|---|
| 201 | TSNHQGIQGV SGTPSLNLH* |

The cp7375 nucleotide sequence <SEQ ID 354> is:

| | |
|---|---|
| 1 | ATGCAACGTA TCATCATTGT AGGAATCGAC ACTGGCGTAG |

GAAAAACCAT

| | |
|---|---|
| 51 | TGTCAGTGCT ATCCTTGCTA GAGCACTTAA CGCAGAATAC |

TGGAAACCTA

| | |
|---|---|
| 101 | TACAAGCAGG GAATCTAGAA AATTCAGATA GCAATATTGT |

TCATGAGCTA

| | |
|---|---|
| 151 | TCGGGAGCCT ACTGTCATCC CGAAGCTTAT CGATTGCATA |

AGCCCTTGTC

| | |
|---|---|
| 201 | TCCACACAAG GCAGCGCAAA TCGATAATGT AAGTATCGAA |

GAGAGTCATA

| | |
|---|---|
| 251 | TTTGTGCGCC AAAAACAACT TCGAATCTGA TTATTGAGAC |

TTCAGGAGGA

| | |
|---|---|
| 301 | TTTTTATCCC CCTGCACATC AAAAAGACTT CAGGGAGATG |

TGTTTTCTTC

| | |
|---|---|
| 351 | TTGGTCATGT TCTTGGATTT TAGTGAGCCA AGCATATCTC |

GGAAGTATCA

| | |
|---|---|
| 401 | ATCACACCTG TTTAACGGTA GAAGCAATGC GCTCACGAAA |

CCTCAATATC

| | |
|---|---|
| 451 | TTAGGTATGG TGGTAAATGG GTATCCAGAG GACGAAGAGC |

ACTGGCTAAC

| | |
|---|---|
| 501 | TCAAGAAATC AAGCTTCCTA TAATCGGGAC TCTTGCCAAG |

GAAAAAGAAA

| | |
|---|---|
| 551 | TCACAAAGAC AATCATAAGC TGTTATGCCG AACAATGGAA |

GGAAGTATGG

| | |
|---|---|
| 601 | ACAAGCAATC ATCAGGGAAT TCAGGGTGTA TCTGGCACCC |

CTTCACTCAA

| | |
|---|---|
| 651 | TCTGCATTAG |

The PSORT algorithm predicts cytoplasm (0.0049).

The following C. pneumoniae protein (PID 4377388) was also expressed <SEQ ID 355; cp7388>:

| | |
|---|---|
| 1 | MQVLLSPQLP PPPQHSVGSI SSPSKLRVLA ITELVFGMLL |

LISGALFLTL

| | |
|---|---|
| 51 | GIPGLSAAIS FGLGIGLSAL GGVLMISGLL CLLVKREIPT |

VRPEEIPEGV

| | |
|---|---|
| 101 | SLAPSEEPAL QAAQKTLAQL PKELDQLDTD IQFVFACLRK |

LKDSKYESRS

| | |
|---|---|
| 151 | FLNDAKKELR VFDFVVEDTL SEIFELRQIV AQEGWDLNFL |

INGGRSLMMT

| | |
|---|---|
| 201 | AESESLDLFH VSKRLGYLPS GDVRGEGLKK SAKEIVARLM |

SLHCEIHKVA

| | |
|---|---|
| 251 | VAFDRNSYAH AEKAFAKALG ALEESVYRSL TQSYRDKFLE |

SERAKIPWNG

| | |
|---|---|
| 301 | HITWLRDDAK SGCAEKKLRD AEERWKKFRK AVFWVEEDGG |

FDINNLLGDW

| | |
|---|---|
| 351 | GTVLDPYRQE RMDEITFHEL YEKTTFLKRL HRKCALAKTT |

FEKKRSKKNL

| | |
|---|---|
| 401 | QAVEEANARR LKYVRDWYDQ EFQKAGERLE KLHALYPEVS |

VSIRENKIQE

| | |
|---|---|
| 451 | TRSNLEKAYE AIEENYRCCV REQEDYWKEE EKREAEFRER |

GNKILSPEEL

| | |
|---|---|
| 501 | ESSLEQFDHG LKNFSEKLME LEGHILKLQK EATAEVENKI |

LSDAESRLEI

| | |
|---|---|
| 551 | VFEDVKEMPC RIEEIEKTLR MAELPLLPTK KAFEKACSQY |

NSCAEMLEKV

| | |
|---|---|
| 601 | KPYCKESLAY VTSKERLVSL DEDLRRAYTE CQKRFQGDSG |

LESEVRACRE

| | |
|---|---|
| 651 | QLRERIQEPE TQGLDLVEKE LLCVSSRLRN TECDCVSGVK |

KEAPPGKKFY

| | |
|---|---|
| 701 | AQYYDEIYRV RVQSRWMTMS ERLEEGVQAC NKMLKAGLSE |

EDKVLKEEEY

| | |
|---|---|
| 751 | WLYREERINK EKRLVGTKIV ATQQRVAAFE SIEVPEIPEA |

PEEKPSLLDK

| | |
|---|---|
| 801 | ARSLPTREDH T |

The cp7388 nucleotide sequence <SEQ ID 356> is:

| | |
|---|---|
| 1 | ATGCAAGTAC TTCTATCTCC GCAGCTACCC CCCCCCCCCC AACACTCTGT |
| 51 | AGGGTCGATT TCTTCTCCAT CTAAACTTCG CGTTTTAGCG ATTACTTTTT |
| 101 | TAGTTTTTGG TATGCTCTTA CTGATTTCAG GAGCTCTCTT TCTGACGTTA |
| 151 | GGGATTCCAG GATTGAGTGC AGCAATTTCT TTTGGATTAG GCATCGGTCT |
| 201 | CTCCGCATTA GGAGGAGTGC TGATGATTTC GGGACTACTA TGTCTTTTAG |
| 251 | TAAAACGAGA GATTCCGACA GTACGACCAG AAGAAATTCC TGAAGGGGTT |
| 301 | TCGCTGGCTC CTTCTGAGGA GCCAGCTCTA CAGGCAGCTC AGAAGACTTT |
| 351 | AGCTCAGCTG CCTAAGGAAT TGGATCAGTT AGATACAGAT ATTCAGGAAG |
| 401 | TGTTCGCATG TTTAAGAAAG CTGAAAGATT CTAAGTATGA AAGTCGAAGT |
| 451 | TTTTTAAACG ATGCTAAGAA GGAGCTTCGA GTTTTTGACT TTGTGGTTGA |
| 501 | GGATACCCTC TCGGAGATTT TCGAGTTGCG GCAGATTQTG GCTCAAGAGG |
| 551 | GATGGGATTT AAACTTTTTG ATCAATGGGG GACGAAGCCT CATGATGACT |
| 601 | GCAGAATCTG AATCGCTTGA TTTGTTTCAT GTATCGAAGC GGCTAGGGTA |
| 651 | TTTACCTTCT GGGGATGTTC GAGGGGAGGG GTTAAAGAAA TCTGCGAAGG |
| 701 | AGATAGTCGC TCGTTTGATG AGCTTGCATT GCGAGATTCA CAAGGTGGCG |
| 751 | GTAGCGTTTG ATAGGAATTC CTATGCGATG GCAGAAAAGG CGTTTGCGAA |
| 801 | AGCGTTGGGA GCTTTAGAAG AGAGTGTGTA TCGGAGTCTT ACGCAGAGTT |
| 851 | ATAGAGATAA ATTTTTGGAG AGCGAGAGGG CGAAGATCCC ATGGAATGGG |
| 901 | CATATAACCT GGTTAAGAGA TGATGCGAAG AGTGGGTGTG CTGAAAAGAA |
| 951 | GCTTCGGGAT GCCGAGGAAC GTTGGAAGAA ATTTAGGAAA GCAGTCTTTT |
| 1001 | GGGTAGAAGA AGACGGGGGC TTTGACATCA ATAATCTCCT TGGAGACTGG |
| 1051 | GGGACAGTGC TTGATCCTTA TAGACAAGAG AGAATGGACG AGATAACGTT |
| 1101 | CCATGAGTTG TATGAAAAAA CTACGTTTTT GAAAAGACTG CACAGAAAGT |
| 1151 | GTGCGTTAGC GAAAACAACC TTTGAAAAGA AGAGATCTAA AAAGAATTTG |
| 1201 | CAGGCAGTCG AGGAGGCGAA TGCACGTAGG TTGAAATATG TAAGGGATTG |
| 1251 | GTATGATCAG GAGTTTCAGA AAGCAGGGGA GAGATTAGAG AAACTGCATG |
| 1301 | CTTTGTATCC TGAGGTTTCA GTCTCTATAA GAGAGAACAA AATACAAGAG |
| 1351 | ACGCGCTCTA ATTTAGAGAA AGCCTATGAG GCTATCGAAG AGAACTATCG |
| 1401 | TTGCTGTGTC CGAGAGCAAG AGGACTACTG GAAAGAAGAA GAGAAAAGGG |
| 1451 | AAGCGGAGTT TAGGGAGAGG GGAAACAAGA TTCTTTCTCC TGAGGAGCTG |
| 1501 | GAAAGTTCTT TGGAGCAATT CGACCATGGT TTGAAAAATT TTTCTGAGAA |
| 1551 | ATTAATGGAA TTGGAAGGGC ATATCTTAAA ACTTCAGAAA GAAGCCACAG |
| 1601 | CAGAGGTGGA GAATAAAATA CTTTCAGATG CAGAGAGCCG CCTTGAGATT |
| 1651 | GTATTTGAAG ATGTCAAGGA GATGCCCTGT CGAATTGAGG AGATAGAGAA |
| 1701 | GACGCTGCGT ATGGCGGAGC TGCCCCTACT TCCTACGAAG AAGGCGTTTG |
| 1751 | AGAAGGCCTG CTCACAATAT AATAGCTGCG CAGAGATGTT GGAGAAGGTG |
| 1801 | AAGCCTTACT GCAAGGAGAG CCTCGCCTAT GTGACTAGCA AAGAGCGTTT |
| 1851 | AGTGAGCTTG GATGAAGATT TACGACGAGC CTACACAGAG TGTCAGAAGA |
| 1901 | GATTCCAGGG GGATTCGGGT TTGGAGTCGG AAGTAAGAGC CTGTCGAGAG |
| 1951 | CAACTGCGAG AGCGGATCCA AGAGTTTGAA ACTCAAGGGC TGGACTTGGT |
| 2001 | GGAAAAAGAG TTGCTTTGTG TGAGTAGTAG ATTAAGAAAT ACAGAGTGCG |
| 2051 | ATTGTGTATC TGGTGTTAAG AAAGAAGCAC CTCCTGGTAA GAAGTTTTAT |
| 2101 | GCCCAGTATT ATGATGAGAT TTATCGAGTT AGAGTTCAAT CCCGATGGAT |
| 2151 | GACGATGTCT GAGAGATTGA GAGAGGGAGT TCAAGCATGC AACAAGATGT |
| 2201 | TGAAGGCAGG CCTAAGCGAA GAAGATAAGG TTCTTAAAGA AGAAGAGTAT |
| 2251 | TGGTTGTATC GAGAGGAGAG AAAGAATAAA GAGAAACGTT TGGTTGGTAC |
| 2301 | TAAGATAGTA GCAACGCAGC AGCGAGTTGC AGCATTTGAA TCCATAGAAG |
| 2351 | TTCCTGAGAT TCCTGAGGCC CCAGAGGAGA AACCGAGTTT GCTGGATAAA |
| 2401 | GCGCGTTCTT TATTTACTCG CGAGGACCAT ACCTAG |

The PSORT algorithm predicts inner membrane (0.461).

The proteins were expressed in E. coli and purified as his-tag products (FIG. 174: 7200lanes 2-3; 7236=lanes 45; 7268=lanes 6-8; 7375=lanes 9-10; 7388=lanes 11-12). The recombinant proteins were used to immunise rice, whose sera were used in Western blots (FIGS. 174, 175, 176, 177 & 178) and for FACS analysis.

These experiments show that cp7200, cp7235, cp7268, cp7375 & cp7388 are surface-exposed and immunoaccessible proteins and that they are useful immunogens. These properties are not evident from the sequence alone.

### Example 179

The following C. pneumoniae protein (PID 4376723) was expressed <SEQ ID 357; cp6723>:

| | |
|---|---|
| 1 | MATSVAPSPV PESSPLSHAT EVLNLPNAYI TQPHPIPAAP |

WETFRSKLST

| | |
|---|---|
| 51 | KHTLCFALTL LLTLGGTISA GYAGYTGNWI ICGIGLGIIV |

LTLILALLLA

| | |
|---|---|
| 101 | IPLKNKQTGT KLIDEISQDI SSIGSGFVQR YGLMFSTIKS |

VHLPELTTQN

| | |
|---|---|
| 151 | QEKTRILNEI EAKKESIQNL ELKITECQNK LAQKQPKRKS |

SQKSFMRSIK

| | |
|---|---|
| 201 | HLSRNPVILF DC* |

The cp6723 nucleotide sequence <SEQ ID 358> is:

| | |
|---|---|
| 1 | ATGCCAACTT CCGTAGCCCC ATCACCAGTC CCCGAGAGCA |

GCCCTCTCTC

| | |
|---|---|
| 51 | TCATGCTACA GAAGTTCTCA ATCTTCCTAA TGCTTATATT |

ACGCAGCCTC

| | |
|---|---|
| 101 | ATCCGATTCC AGCGGCTCCT TGGGAGACCT TTCGCTCCAA |

ACTTTCCACA

| | |
|---|---|
| 151 | AAGCATACGC TCTGTTTTGC CTTAACACTA CTGTTAACCT |

TAGGGGGAAC

| | |
|---|---|
| 201 | GATCTCAGCA GGTTACGCAG GATATACTGT AAACTGGATC |

ATCTGTGGCA

| | |
|---|---|
| 251 | TCGGCTTGGG AATTATCGTA CTCACACTGA TTCTTGCTCT |

TCTTCTAGCA

| | |
|---|---|
| 301 | ATCCCTCTTA AAAATAAGCA GACAGGAACA AAACTGATTG |

ATGAGATATC

| | |
|---|---|
| 351 | TCAAGACATT TCCTCTATAG GATCAGGATT TGTTCAGAGA |

TACGGGTTGA

| | |
|---|---|
| 401 | TGTTCTCTAC AATTAAAAGC GTGCATCTTC CAGAGCTGAC |

AACACAAAAT

| | |
|---|---|
| 451 | CAAGAAAAAA CAAGATTTTT AAATGAAATT GAAGCGAAAA |

AGGAATCGAT

| | |
|---|---|
| 501 | CCAAAATCTT GAGCTTAAAA TTACTGAGTG CCAAAACAAG |

TTAGCACAGA

| | |
|---|---|
| 551 | AACAGCCGAA ACGGAAATCA TCTCAGAAAT CATTTATGCG |

TAGTATTAAG

| | |
|---|---|
| 601 | CACCTCTCCA AGAACCCTGT AATTTTGTTC GATTGCTGA |

The PSORT algorithm predicts inner membrane (0.6095).

The protein was expressed in E. coli and purified as a his-tag product (FIG. 179A). The recombinant protein was used to immunise mice, whose sera were used in a Western blot (FIG. 179B) and for FACS analysis.

These experiments show that. cp6723 is a surface-exposed and immunoaccessible protein, and that it is a useful immunogen. These properties are not evident from the sequence alone.

### Example 180

The following C. pneumoniae protein (PID 4376749) was expressed <SEQ ID 359; cp6749>:

| | |
|---|---|
| 1 | MSYYFSLWYL KVQQHFQAAF DFTRSLCSRI SNFALGVIAL |

LPIIGQLYVG

| | |
|---|---|
| 51 | LDWLLSRIKK PEFPSDVDQI VRVEHVVGHD HRSRVEDILK |

RQRLSLEPRD

| | |
|---|---|
| 101 | EGKVHGDLPS APFF* |

The cp6749 nucleotide sequence <SEQ ID 360> is:

| | |
|---|---|
| 1 | ATGAGTTATT ACTTTTCTCT TTGGTATCTG AAGGTGCAAC |

AGCACTTTCA

| | |
|---|---|
| 51 | AGCAGCATTT GATTTTACTC GCTCCCTGTG TTCACGAATT |

TCTAATTTTG

| | |
|---|---|
| 101 | CTTTGGGAGT GATTGCATTG CTTCCTATTA TTGGGCAGTT |

GTATGTAGGG

| | |
|---|---|
| 151 | CTGGACTGGC TCCTCTCTAG GATAAAAAAG CCAGAATTTC |

CTTCCGATGT

| | |
|---|---|
| 201 | GGATCAGATC GTGCGAGTAG AACACGTCGT GGGTCACGAC |

CATAGAAGTC

| | |
|---|---|
| 251 | GAGTTGAAGA TATTCTAAAG AGACAAAGGC TCTCATTAGA |

GCCTAGAGAC

| | |
|---|---|
| 301 | GAGGGGAAGG TTCACGGAGA TCTGCCTTCA GCTCCTTTTT |

TTTGA

The PSORT algorithm predicts inner membrane (0.2996).

The protein was expressed in E. coli and purified as a his-tag product (FIG. 180A). The recombinant protein was used to immunise mice, whose sera were used in a Western blot (FIG. 180B) and for FACS analysis.

These experiments show that cp6749 is a surface-exposed and immunoaccessible protein, and that it is a useful immunogen. These properties are not evident from the sequence alone.

### Example 181 Example 182, Example 183, Example 184 and Example 185

The following C. pneumoniae protein (PID 4376301) was expressed <SEQ ID 361; cp6301>:

| | |
|---|---|
| 1 | LNQDLQNVYQ ECQKATGLES EVSAYELHLR EQXTEFETQG |

LDVIKEELLF

| | |
|---|---|
| 51 | VSSTLKSKLS YDPLIADIPC MKFYEEYYDG IDKARVQSRW |

LEKSERYRKA

| | |
|---|---|
| 101 | KKGFQEMLKE GLFKEDQALK KAEYRLLREK RMNKEKLLIC |

NKIEAAQQRV

| | |
|---|---|
| 151 | QEFGPSDS* |

The cp6301 nucleotide sequence <SEQ ID 362> is:

| | |
|---|---|
| 1 | TTGAATCAGC ATTTACAAAA TGTATACCAA GAGTGCCAGA |

AGGCTACAGG

| | |
|---|---|
| 51 | TTTAGAATCG GAAGTGAGTG CATATAGAGA TCATCTTAGA |

GAGCAGATCA

| | |
|---|---|
| 101 | CAGAGTTTGA AACTCAAGGG CTGGACGTGA TAAAAGAAGA |

ACTTCTTTTT

| | |
|---|---|
| 151 | GTGAGTAGTA CTCTCAAAAG TAAATTGAGC TATGATCCAT |

TAATAGCAGA

| | |
|---|---|
| 201 | CATTCCCTGT ATGAAGTTTT ATGAGGAGTA TTATGATGGC |

ATTGATAAAG

| | |
|---|---|
| 251 | CGAGAGTTCA ATCCCGATGG CTGGAGAAGT CTGAGAGGTA |

TAGAAAGGCG

| | |
|---|---|
| 301 | AAGAAGGGAT TCCAAGAGAT GCTGAAGGAA GGCCTATTCA |

AAGAAGATCA

| | |
|---|---|
| 351 | GGCTTTGAAA AAAGCAGAGT ATAGATTACT TCGAGAGAAG |

AGAATGAATA

| | |
|---|---|
| 401 | AGGAGAAGCT TTTGATTTGC AATAAGATAG AAGCAGCTCA |

GCAGCGAGTC

| | |
|---|---|
| 451 | CAAGAATTTG GACCCTCGGA TTCATAA |

The PSORT algorithm predicts cytoplasm (0.4621).

The following C. pneumoniae protein (PID 4376558) was also expressed <SEQ ID 363; cp6558>:

| | |
|---|---|
| 1 | MNIPAPQVPV IDEPVVNNTS SYGLSLKSSL RPITYLILAI |

LAIATLMSVL

| | |
|---|---|
| 51 | YFCGIXSVGT FVLGMLIPLS VCSVLCVAYL FYQQSSIEKT |

KVTSITSPSV

| | |
|---|---|
| 101 | FFSDEDLNLL LGREEDSVSA IDELLKNFPA DDFRRPITT |

YSNFLDEQGR

| | |
|---|---|
| 151 | PNESREEDSH TSKIL* |

The cp6558 nucleotide sequence <SEQ ID 364> is:

| | |
|---|---|
| 1 | ATGAACATAC CCGCTCCCCA AGTACCAGTC ATAGATGAGC |

CPGTAGTGAA

| | |
|---|---|
| 51 | CAACACAAGT AGCTATGGTC TTTCATTGAA AAGTAGTTTA |

AGACCGATTA

| | |
|---|---|
| 101 | CTTATTTGAT TTTAGCTATC TTAGCTATAG CCACACTGAT |

GTCTGTTCTC

| | |
|---|---|
| 151 | TACTTTTGTG GCATCATTAT TGTTGGGACG TTTGTTTTGG |

GCATGCTGAT

| | |
|---|---|
| 201 | CCCTCThTCG GTCTGCTCTG TTCTTTGCGT TGCCTATTTA |

TTCTATCAGC

| | |
|---|---|
| 251 | AATCTTCThT AGAAAAGACT AAGGTCTTTT CTATAACCAG |

TCCTTCAGTA

| | |
|---|---|
| 301 | TTTTTCTCTT ATGAGGATCT TAATTTACTC TTAGGTCGAG |

AAQAAGATTC

| | |
|---|---|
| 351 | AGTGTCTGCA ATTGATGAAC TTCPTAAGAA CTTTCCAGCT |

GATGATTTCC

| | |
|---|---|
| 401 | GTAGGCCGAA GATGCTTCCT TATTCAAATT TTCTAGATGA |

GCAGGGAAGG

| | |
|---|---|
| 451 | CCTAAIGAGA GTAGGGAAGA AGACTCTCAT ACTTCCAAGA |

TCTTATAA

The PSORT algorithm predicts inner membrane (0.4630).

The following C. pneumoniae protein (PID 4376630) was also expressed <SEQ ID 365; cp6630>:

| | |
|---|---|
| 1 | MSMTIVPHAL FKNHCECHST FPLSSRTIVR IAIASLFCIG |

ALAALGCLAP

| | |
|---|---|
| 51 | PVSYIVUSVL APIAFVILSL VILALIFGEK KLPPTTRIIP |

DRFTHVIDTA

| | |
|---|---|
| 101 | YGLSISAFVR EQQVTIAEFR QFSTALLCNI SPEEKIRQLP |

SELRSKVEST

| | |
|---|---|
| 151 | GISPLAGDLE KNNWPIFEDL LSQTCPLYWL QKFISAGDPQ |

VCRDLGVPRE

| | |
|---|---|
| 201 | CYGYYWLGPL GYSTAXAPIP CKETHHILQQ LTKEDVLLLK |

NKALQERWDT

| | |
|---|---|
| 251 | DEVKAIVTRI YTTYTARGTL KTEAGGLTKB TISKELLLLS |

LHGYSFDQLQ

| | |
|---|---|
| 301 | LITQLPRDAW DWLCPVDNST AYNLQLCALV GALSSQNLLD |

EBSIDFDVNL

| | |
|---|---|
| 351 | GLYVIQDLKE AVQAPSASDE PKKELGKFLL RHLSSVSKEL |

ESVLRQGLHR

| | |
|---|---|
| 401 | IALEHGNARA RVYDVNFVTQ ARIHRKTSIF FKD* |

The cp6630 nucleotide sequence <SEQ ID 366> is:

| | |
|---|---|
| 1 | ATGAGCATGA CGATCGTTCC ACATGCTTTA TTTAAAAATC |

ATTGCGAGTG

| | |
|---|---|
| 51 | TCATTCTACC TTTCCTTTGA GTTCAAGGAC TATTGTAAGT |

ATAGCCATTG

| | |
|---|---|
| 101 | CCAGCCTCTT TTGTATTGGT GCATTAGCAG CTTTAGCCTG |

TTTGGCTCCT

| | |
|---|---|
| 151 | CCCGTTTCTT ATATTGTTGG GAGTGTTTTA GCTTTTATTG |

CCTTTGTCAT

| | |
|---|---|
| 201 | TCTTTCTTTA GTAATTTTAG CTTTGATTTT TGGAGAGAAG |

AAGCTTCCAC

| | |
|---|---|
| 251 | CAACACCAAG AATCATTCCT QATATATTTA CTCACGTGAT |

AGATQTQCJT

| | |
|---|---|
| 301 | TATGGCCTTT CAATCTCTGC ATTTGTAAGA GAACAGCAGQ |

TAACATTAGC

| | |
|---|---|
| 351 | CGAGTTTAGA CAATTTTCTA CTGCCCTCTT GTGTAACATA |

TCTCCTGAAG

| | |
|---|---|
| 401 | AGAAAATCAA ACAATTCCCT TCTGAATTGC GAAGTAAAGT |

AGAGAGTTTT

| | |
|---|---|
| 451 | GGTATTAGCA GGCTCGCAGG TGATTTAGAA AAGAATAATT |

GGCCAATATT

| | |
|---|---|
| 501 | TGAAGATCTT TTAAGCCAAA CCTTCCCGTT ATATTGGCTT |

CAGAAATTTA

| | |
|---|---|
| 551 | TATCAGCAGG AGATCCACAA GTTTGTAGAG ACCTAGGTGT |

CCCTAGAGAA

| | |
|---|---|
| 601 | TGTTATGGGT ACTATTGGCT AGGGCCTTTG CGATACAGTA |

CAGCTAAGGC

| | |
|---|---|
| 651 | TACAATTTTT TGTAAAGAGA CGCATCATAT TCTTCAACAA |

TTAACGAAAG

| | |
|---|---|
| 701 | AGGACGTTCT TTTATTAAAA AACAAGGCTC TTCAAGAGAA |

ATGGGATACT

| | |
|---|---|
| 751 | GATGAAGTCA AAGCAATTGT AGAGCGTATC TACACTACCT |

ATACGGCACG

| | |
|---|---|
| 801 | AGGAACTCTA AAGACCGAAG CAGGGGTACT TACAAAAGAG |

ACAATCAGTA

| | |
|---|---|
| 851 | AGGAATTGCT ATTGTTGAGC TTGCATGGCT ATTCTTTTGA |

TCAGCTACAG

| | |
|---|---|
| 901 | CTGATCACTC AACTTOCTAG AGATGCTTTG GATTGGCTGT |

GTTTTGTAGA

| | |
|---|---|
| 951 | TAACAGTACC GCATACAACC TTCAGCTTTG TGCTCTTGTA |

GGAGCTTTGT

| | |
|---|---|
| 1001 | CATCCCAAAA TCTTCTTGAC GAATCTTCTA TCGATTTTGA |

TGTAAACCTA

| | |
|---|---|
| 1051 | GGCCTGTATG TGATTCAGGA TCTAAAAGAA TCTGTTCAAG |

CATTTTCTGC

| | |
|---|---|
| 1101 | TTCTGATGAG CCAAAGAAAG AACTAGGTAA ATTCTTGTTA |

AGGCATTTGA

| | |
|---|---|
| 1151 | GTTCAGTTTC TAAGCGATTA GAGAGTGTAT TAAGACAGGG |

TCTTCACAGA

| | |
|---|---|
| 1201 | ATAGCTCTAG AGCATGGAAA TGCCAGAGCT ATGGTTTATG |

ACGTCAATTT

| | |
|---|---|
| 1251 | TGTAACAGGA GCTAGAATTC ATAGGAAGAC GAGThTCTTC |

TTTAAAGACT

| | |
|---|---|
| 1301 | AA |

The PSORT algorithm predicts inner membrane (0.7092).

The following C. pneumoniae protein (PID 4376633) was also expressed <SEQ ID 367; cp6633>:

| | |
|---|---|
| 1 | MVNIQPVYRW TQVNYSQATQ FSVCQPALSL IIVSVVAAVL |

AIVALVCSQS

| | |
|---|---|
| 51 | LLSIELGTAL VLVSLILFAS AMFTMIYTRQ BPKBLLIPKK |

IMTLIQEHYP

| | |
|---|---|
| 101 | SIVVDFIRDQ EVSIYEIHHL ISILTRTNVF DKAPVYLQEK |

LLQFGIEKFK

| | |
|---|---|
| 151 | DVHPSKLPNF EEILLQHCPL HWLGRLVYPM VSDVTTGTYG |

YYWCGPLGLY

| | |
|---|---|
| 201 | ENAPSLFERR SLLLLKKISF GEFALLEDGL KTNTWSSSEL |

VQIRQNLFTR

| | |
|---|---|
| 251 | YYADKTEVDE AELNADYEQE DSLLHLIFSH KLS* |

The cp6633 nucleotide sequence <SEQ ID 368> is:

| | |
|---|---|
| 1 | ATGGTTAATA TACAGCCTGT GTATAGQAAT ACCCAAGTCA |

ACTATAGTCA

| | |
|---|---|
| 51 | GGCTACCCAA TTTTCGGTGT GCCAGCCAGC GCTTAOCCTG |

ATTATCGTTT

| | |
|---|---|
| 101 | CTGTTGTTGC TGCTGTACTC GCTATTGTAG CTTTGGTATG |

CAGTCAATCT

| | |
|---|---|
| 151 | CTTTTATCCA TAGAGTTAGG AACTGCTCTT GTTCTAGPTT |

CTCTTATTCT

| | |
|---|---|
| 201 | TTTTGCTTCT GCTATGTTTA TGATTTATAA GATGAGACAA |

GAACCTAAGG

| | |
|---|---|
| 251 | AGTTGCTGAT CCCTAAGAAA ATCATGGAAC TCATCCAAGA |

ACATTATCCA

| | |
|---|---|
| 301 | AGTAATGTTG TTGATTTTAT TAGAGATCAG GAGGTTTCCA |

TTTATGAGAT

| | |
|---|---|
| 351 | ACATCACTTG ATCTCTATTC TTAATAAGAC GAATGTTTTC |

GACAAAGCAC

| | |
|---|---|
| 401 | CAGTATATTT ACAAGAAAAA CTCTTACAGT TTGGCATTGA |

GAAGTTCAAA

| | |
|---|---|
| 451 | GATGTACATC CAAGTAAGCT CCCTAATTTT GAAGAAATTC |

TTCTACAGCA

| | |
|---|---|
| 501 | TTGCCCATTG CATTGGTTGG GACGTCTGGT ATATCCCATG |

GTATCGGATG

| | |
|---|---|
| 551 | TCACTCCAGG AACCTATGGA TACTATTGGT GTGGTCCTTT |

AGGACTGTTC

| | |
|---|---|
| 601 | GAGAACGCTC CCTCTCTTTT TGAACGTCGA TCTCTTCTAT |

TGTTAAAGAA

| | |
|---|---|
| 651 | AATTAGCTTT GGAGAGTTTG CTCTTTTAQA AGWTGGTCTC |

AAGAAAAACA

| | |
|---|---|
| 701 | CGTGGAGTTC TTCGGAACTC GTTCAAATCT GACAAAACCT |

TTTTACAAGA

| | |
|---|---|
| 751 | TATTATGCTG ATAAAGAAGA GGTAGATGAA GCAGAGTTAA |

ACTCTGATTA

| | |
|---|---|
| 801 | CGAACAGTDT GATTCCCTCC TTCACCTTAT TTTTTCTCAC |

AAGCTCTCTT

| | |
|---|---|
| 851 | GA |

The PSORT algorithm predicts inner membrane (0.7283).

The following C. pneumoniae protein (PID 4376642) was also expressed <SEQ ID 369; cp6642>:

| | |
|---|---|
| 1 | MATISPISLT VDHPLVDTKK KSCSNFDKIQ SRILLITAIF AVLVTIGTLL |
| 51 | IGLLLNIPVI YFLTGISFIA VVLSNFILYX RATTLLRPRA CGKHKEIKPK |
| 101 | RVSTNLQYSS ISIAINRSKE NWEHQPKDLQ NLPAPSALLT DNPYEIWKAX |
| 151 | HSLFSLVSLL PGGNPEHLLI SASENLGKTL LIEETSQNAP ISSYVDTTPS |
| 201 | PRSLLNEAIQ ETRVEINTEL PAGDSGERLY WQPDFRGRVF LPQIPTTPEA |
| 251 | IYQYYYALYV TYIQTAINTN TQIIQIPLYS LREHLYSREL PPQSRMQQSL |
| 301 | AMITAVKYMA ELHPEYPLTI ACVERSLAQL PQESIEDLS* |

The cp6642 nucleotide sequence <SEQ ID 370> is:

| | |
|---|---|
| 1 | ATGGCTACAA TCTCACCCAT ATCTTTAACT GTAGATCATC CCCTAGTAGA |
| 51 | CACTAAAAAA AAATCCTGCA QCAACTTTGA TAAGATTCAG TCTCGAATTC |
| 101 | TATTGATTAC TGCAATCTTT GCTGTCTTAQ TTACTATAGG GACCCTACTT |
| 151 | ATTGGTTTGC TTTTAAATAT TCCTGTTATC TATTTCCTCA CAGGAATTTC |
| 201 | ATTTATTGCT GTTGTTCTTA GCAACTTTAT CCTTTATAAA CGAGCAACCA |
| 251 | CCCTCTTAAA ACCGCGTGCT TGTGGCAAAC ACAAAGAAAT AAAACCAAAA |
| 301 | AGGGTCTCCA CCAACCTACA GTATTCTTCT ATCTCTATCG CAATCAATCG |
| 351 | TTCTAAAGAA AACTGGGAAC ACCAACCCAA GGACCTACAG AATCTCCCCG |
| 401 | CACCCTCTGC ATTACTCACA GATAACCCTT ACGAGATATG GAAAGCTAAA |
| 451 | CATTCACTGT TTTCCCTAGT ATCCCTCCTA CCGGGAGQCA ATCCAGAACA |
| 501 | TCTCTTAATT TCAGCTTCCG AAAATTTAGG AAAGACTCTG TTAATTGAAG |
| 551 | AAACCTCGCA AAATGCGCCT AWATCCTCCT ACGTAGAQAC CACTCCCTCC |
| 601 | CCAAAATCCT TGCTCAATGA GGCAATTCAG GAAACCAGGG TAGAAATAAA |
| 651 | TACAGAACTC CCTGCGGGAG ATTCAGGAGA ACGTTTATAC TGGCAACCCG |
| 701 | ATTTCCGAGG CCGCGTCTTC CTCCCACAAA TACCAACAAC TCCTGAAGCC |
| 751 | ATCTACCAAT ACTACTATGC ACTCTATGTC ACTTATATCC AGACAGCGAT |
| 801 | CAATACGAAC ACCCAAATTA TCCAAATCCC TTTATACAAC TTGAGGGAGC |
| 851 | ATCTCTATTC TAGAGAATTG CCCCCGCAAT CAAGAATGCA ACAATCTTTG |
| 901 | GCTATGATTA CAGCAGTAAA ATACATGGCC GAGCTGCACC CAGAATATCC |
| 951 | GCTAACTATT GCTTGTGTTG AAAGATCCTT AGCCCAACTA CCTCAAGAAA |
| 1001 | GTATTGAGGA TCTCTCTTAG |

The PSORT algorithm predicts inner membrane (0.5288).

The proteins were expressed in E. coli and purified as GST-fusion products. The recombinant proteins were used to immunise mice, whose sera were used in Western blots (FIGS. 181-185) and for FACS analysis.

These experiments show that cp6301, cp6558, cp6630, cp6633 and cp6642 are surface-exposed and immunoaccessible proteins, and that they are useful immunogens. These properties are not evident from their sequences alone.

### Example 186

The following C. pneumoniae protein (PID 4376389) was expressed <SEQ ID 371; cp6389>:

| | |
|---|---|
| 1 | MSEVKPLFLK NDSFDLATQR FQNLINMLQA QAEIYNEYHE KNARVQNEIK |
| 51 | EQKDFVKRCI EDFEMAGLGV LKEELASLTR DFHDKAKAET SMLIECPCIG |
| 101 | FYYSIHQEEQ RQEQERLQKM AERYRDCKQV LEAVQVEQKD MISSRVVVDD |
| 151 | SYFEEEKEEQ DVKNEKKEQD * |

The cp6389 nucleotide sequence <SEQ ID 372> is:

| | |
|---|---|
| 1 | ATGDCAGAAG TGAAGCCTTT GTTTTTAAAG AATGACTCTT TTGATTTGGC |
| 51 | AACTCAGAGA TTCCAGAATC TAATTAACAT GCTACAAGAG CAAGCCGAGA |
| 101 | TATATAACGA GTATGAAGAA AAGAATGCTA GGGTTCWJAA TGAGATTAAG |
| 151 | GAGCAAAAGG ACTTTGTGAA AAGATGCATA GAGGACTTTG AAGCCAGAGG |
| 201 | ACTGGGGGTG CTAAAAGAAG AGCTTGCATC TTTGACGCGT GATTTCCATG |
| 251 | ATAAAGCAAA AGCAGAGACT TCTATGCTCA TTGAATGTCC TTGTATTGGT |
| 301 | TTTTATTATA GTATTCATCA GGAGGAACAA AGGCAAAGGC AAGAAAGGCT |
| 351 | TCAAAAGATG GOTGAGCGCT ATAGGGACTG TAAACAAGTC TTGGAGGCTG |
| 401 | TCCAGQTGGA GCAAAAAGAT ATGATATCTT CTAGAGTCGT TGTCGATGAC |
| 451 | AGCTACTTTG AAGAAGAAAA AGAAGAACAA AAGGTGGATA ACAGAAAGAA |
| 501 | AGAACAGGAC TAG |

The PSORT algorithm predicts cytoplasm (0.3193).

The protein was expressed in E. coli and purified as a GST-fusion product (FIG. 186A) and also in his-tagged form. The recombinant proteins were used to immunise mice, whose sera were used in a Western blot (FIG. 186B) and for FACS analysis

These experiments show that cp6389 is a surface-exposed and immunoaccessible protein, and that it is a useful immunogen. These properties are not evident from the sequence alone.

### Example 187

The following C. pneumoniae protein (PID 4376792) was expressed <SEQ ID 373; cp6792>:

| | |
|---|---|
| 1 | VLQEHFFLSE DVITLAQQLL GHKLITTHEG LITSGYIVET EAYRGPDDKA |
| 51 | CHAYNYRKTQ RNRAMYLKGG SAYLYRCYGM HHLLNVVTGP EDIPHAVLIR |
| 101 | AILPDQGKEL MIQRRQWRDK PPHLLTNGPG KVCQALGISL ANNRQRLNTP |
| 151 | ALYISKEKIS GTLTATARIG IDYAQEYRDV PWRFLLSPED SGKVLS* |

The cp6792 nucleotide sequence <SEQ ID 374> is:

| | |
|---|---|
| 1 | GTGCTACAAG AACATTTTTT TCTATCGGAA GATGTAATTA CACTAGCGCA |
| 51 | ACAGCTTTTA GGACATAAAC TCATCACAAC ACAAGAGGGA CTGATAACTT |
| 101 | CAGGTTACAT TGTAGAAACC GAAGCGTATC GTGGCCCTGA TGACAAAGCA |
| 151 | TGCCACGCCT ACAACTACAG AAAAACTCAG AGGAACAGAG CGATGTACCT |
| 201 | GAAAGGAGGC TCTGCTTACC TCTACCGTTG CTATGGCAEG CATCACCTAT |
| 251 | TGAATGTTGT CACTGGACCT GAGGACATTC CCCATGCCGT CCTGATCCGG |
| 301 | GCCATCCTTC CTGATCAAGG CAAAGAACTT ATGATCCAAC GCCGCCAATG |
| 351 | GAGAGATAAA CCCCCACACC TTCTCACCAA TGGACCCGGA AAAGTGTGCC |
| 401 | AAGCTCTAGG AATCTCTTTG GAAAACAATA GGCAACGCCT AAATACCCCA |
| 451 | GCTCTCTATA TCAGCAAAGA AAAAATCTCT GGGACTCTAA CAGCAACTGC |
| 501 | CCGGATCGGC ATCQATTATG CTCAAGAGPA TCGTGATGTC CCATGGAGAT |
| 551 | ATCTCCTATC CCCAGAAGAT TCGGGAAAAG TTTTATCTTA A |

The PSORT algorithm predicts cytoplasm (0.180).

The protein was expressed in E. coli and purified as a his-tagged product (FIG. 187A; lanes 2-4). The recombinant protein was used to immunise mice, whose sera were used in a Western blot (FIG. 187B) and for FACS analysis.

These experiments show that cp6792 is a surface-exposed and immunoaccessible protein, and that it is a useful immunogen. These properties are not evident from the sequence alone.

### Example 188

The following C. pneumoniae protein (PID 4376868) was expressed <SEQ ID 375; cp6868>:

| | |
|---|---|
| 1 | MVETVLHNFQ RYLSKYLYRV FRPPCRKKTF LSSHRVLARP SFPVDYCPGK |
| 51 | IYDLQEIYEE LNAQLFQGAL RLQIGWFGRK ATRKGKSVVL GLFHENEQLI |
| 101 | RIHRSLDRQE IPRFFMETAV YHEHVHSVVP REYSLSGRSI FMGKKFKEYE |
| 151 | QRFPLYDRAV AWEKANAYLL RGYKKRVGGG YGRA* |

The cp6868 nucleotide sequence <SEQ ID 376> is:

| | |
|---|---|
| 1 | ATGGTTGAAA CAGTACTTCA TAATTTCCAA CGTTATCTGA GCAAGTATCT |
| 51 | CTATAGGGTA TTTCGCTTCC CATGTCGTAA AAAGACGTTC CTATCTTCGC |
| 101 | ACAGGGTTCT TGCTCGTCCT TCATTCCCAG TAAACTACTG TCCGGGAAAG |
| 151 | ATCTATGATT TCCAGGAGAT CTATGAGGAA TTGAATGCGC AGTTATTTCA |
| 201 | AGGTGCACTG CGTTTACAGA TTGGTTGGTT CGGAAGGAAA GCTACCAGAA |
| 231 | AAGGCAAGAG TGTTGTCTTG GGATTGTTTC ATGAAAATGA ACAGTTAATT |
| 301 | CGAATTCATC GTTCTTTAGA TCGGCAGGAA ATCCCAAGAT TTTTTATGGA |
| 351 | ATATCTTGTG TATCATGAAA TGGTTCATAQ TGTAGTCCCT AGAGAGTATT |
| 401 | CTCTATCGGG GCGTTCGATT TTTCATGGTA AAAAGTTTAA AGAATACGAA |
| 451 | CAACGTTTCC CCTTGTATGA TCGTGCTGTT GCTTGGGAAA AGGCAAACGC |
| 501 | TTATTTATTG CGAGGGTATA AAAAAAGAGT AGGTGGAGGA TATGGCAGGG |
| 551 | CATAG |

The PSORT algorithm predicts bacterial cytoplasm (0.325).

The protein was expressed in E. coli and purified as a his-tag product (FIG. 188A; lanes 2-3). The recombinant protein was used to immunise mice, whose sera were used in a Western blot (FIG. 188B) and for FACS analysis.

These experiments show that cp6868 is a surface-exposed and immunoaccessible protein, and that it is a useful immunogen. These properties are not evident from the sequence alone.

### Example 189

The following C. pneumoniae protein (PID 4376894) was expressed <SEQ ID 377; cp6894>:

| | |
|---|---|
| 1 | MYKRCVLDKI LKGXVAGSLI LLYWSSDLLE RDIKSIKGNV RDIQEDIREI |
| 51 | SRVVKQQQTS QAIPAAPGVM LAPKLVRDEA FALLPGPPSY PNLLSLDPYK |
| 101 | QQTLPELLGT NFHPHGILRA AHVGKPENLS PPNGFDYVVG FYDLCIPSIA |
| 151 | SPHVGKYBEF SPDLAVKIAE HLVEDGSGDK EPHIYLRPNV FWRPIDPAAL |
| 201 | PKHVQLDEVF QRPHPVTAHD IKPFADAVMA PYVATNRAVA LRSCYEDVVS |
| 251 | VSVENDLKLV VRWKAHTVIN EEGKEERKVL YSAFBNTLSL QPLPRFVYQY |
| 301 | PANGEKIIED ENIDTYRTNS IWAQNFTMHW ANNYIVSCGA YAFAGWDDEK |
| 351 | IVFERNPDFY DPLAALIDKR FVYFDESTDS LFQDFKTGKI DIBYLPPNQR |
| 401 | DNFYSPNKSS AYNKQVAKGG AVRATVSADR AYTYIGWNCF SLFPQSRQVA |
| 451 | CAMNMAIDEE RIIEQCLDGQ GYTISGPFAB SSPSYNKQIE GWHYSPERAA |
| 501 | ALLEEEGWTD TDGDGIRBKV IDGVIVPFRF ELCYYVKBVT AHTIADYVAT |
| 551 | AGNEIGIECS LLGLDMADLS QAFDEKNFDA LLMGWCLGIP PEDPRALWHS |
| 601 | EGA4EKGSAN VVGFHNEEAD KIIDRLSYEY DLKERNRLYH AFHEIIHEEA |
| 651 | PYAPLFSRMC SLLYKDYVKN IFVPTHRTDL IPEAQDETVN VTMVWLEKKE |
| 701 | DPCLSTS* |

The cp6894 nucleotide sequence <SEQ ID 378> is:

| | |
|---|---|
| 1 | ATGTATAAAA GATGTGTGCT AGATAAAATT TTAAAGGGGA TTGTCGCCGG |
| 51 | TTCTTTAATT TTGTPATACT GGTCCTCAGA CCTACTTGAA AGAGACATTA |
| 101 | AGTCGATAAA AGGTAACGTA AGAGATATTC AAGAAGACAT TCGTGAAATC |
| 151 | TCACGCGTAG TGAAACAACA GCAGACATCA CAAGCTATCC CTGCGGCACC |
| 201 | TGGGGTGATG CTCGCTCCTA AGCTCGTCAG AGACGAAGCT TTTGCTCTAC |
| 251 | TCTTTGGAGA TCCTAGTTAT CCTAATTTAC TTTCCCTAGA CCCCTATAAA |
| 301 | CAGCAGACTC TTCCTGAACT TCTAGGAACA AATTTCCACC CTCATGGTAT |
| 351 | CCTACGCACT GCCCATGTCG GAAAACCCGA AAATCTGAGC CCTTTTAATG |
| 401 | GCTTTGATTA TGTCGTGGGC TTTTACGATC TCTGTATTCC TAGTTTAGCT |
| 451 | TCTCCCCACG TAGGGAAATA CGAAGAATTT TCTCCAGATC TCGCTGTGAA |
| 501 | AATAGAAGAA CATCTTGTTG AAGATGGTTC TGGGGATAAA GAGTTTCACA |
| 551 | TCTATCTGAG GCCGAATGTT TTTTGGCGTC CTATAGATCC TAAGGCCCTT |
| 601 | CCAAAACACG TTCAGTTAGA CGAAGTATTT CAACGTCCTC ATCCTGTGAC |
| 651 | AGCTCATGAT ATTAAGTTTT TCTACGACGC TGTTATGAAC CCTTATGTAG |
| 701 | CAACCATGCG AGCAGTGGCT CTGCGCTCTT G1TATGAAGA TGTGGTTTCA |
| 751 | GTCTCAGTAG AAAACGATTT AAAATTAGTA GTCAGATGGA AAGCACACAC |
| 801 | GGTAATCAAT GAAGAAGGAA AGGAAGAGCG CAAAATGCTC TACTCTGCAT |
| 851 | TTTCTAATAC CTTAAGCTTG CAGCCCCTCC CTAGATTTGT ATATCAGTAT |
| 901 | TTTGCTAACG GGGAAAAAAT CATTGAAGAT GAGAAPATCG ATACCTACCG |
| 951 | AACCAATTCC ATTTGGGCGC AAAACTTCAC TATGCATTGG GCAAACAACT |
| 1001 | ATATTGTAAG TTGTGGAGCC TACTACTTTG CAGGGATGGA TGATGAGAAA |
| 1051 | ATCGTGTTTT CTAGAAATCC TGACTTCTAT GATCCTCTTG CGGCTCTTAT |
| 1101 | TGACAAGCGT TTCGTCTATT TTAAGGAAAG CACAGACTCC CTATTCCAAG |
| 1151 | ATTTTAAGAC AGGGAAAATA GACATCTCTT ACCTTCCACC CAACCAAAGA |
| 1201 | GATAATTTCT ATAGTTTTAT GAAAAGCTCC GCTTATAACA AACAGGTAGC |
| 1251 | TAAGGGAAGA GCCGTCCGTG AAACAGTCTC AGCAGATCCA GCATATACGT |
| 1301 | ACATAGGATG GAATTACTTT TCATTATCTT TCCAAAGCCG ACAGGTGCGC |
| 1351 | TGTGCTATGA ACATGGCAAT CGATAGAGAG AGGATTATCG AACAGTGCTT |
| 1401 | GGATGGCCAA GGCTATACGA TTAGTGGGCC TTTTGCTTCG AGTTCTCCTT |
| 1451 | CTTATAATAA ACAGATCGAA GGGTGGCATT ATTCTCCAGA AGAAGCAGCT |
| 1501 | CGTCTCCTGG AAGAAGAGGG ATCGATAGAT ACCGATGGCG ATGGAATCCG |
| 1551 | AGAAAAAGTT ATCGATGGTG TGATTGTCCC GTTCCGTTTC CGTTTATGCT |
| 1601 | ATTATGTAAA GAGTGTCACC GCTCATACCA TWCCAGATTA CGTAGCTACT |
| 1651 | GCTTGTAAGG AAATCGGAAT CGAGTGTAGC CTTCTAGGAC TAGATATGGC |
| 1701 | CGATCTTTCG CAAGCTTTTG ATGAAAAGAA TTTCGATGCT CTTTTAATGG |
| 1751 | GATGGTGTTT AGGAATTCCT CCTGAGGATC CTAGGGCTTT ATGGCATTCT |
| 1801 | GAAGGGGCTA AGGAAAAGGG TTCAGCGAAT GTTGTAGGTT TCCATAATGA |
| 1851 | AGAAGCTGAT AAAATCATAG ACAGACTCAG CTACGAATAC GATCTGAAAG |
| 1901 | AACGTAATCG CCTGTACCAC CGTTTCCATG AAATTATTCA TGAGGAAGCT |
| 1951 | CCTTATGCTP TCTTGTTCTC ACGACATTGT TCCTTACTTT ATAAGGATTA |
| 2001 | TGTAAAAAAT ATTTTCATAC CTACACATAG AACAGATTTA ATTCCTGAAG |
| 2051 | CTCAGGATGA GACTGTCAAC GTAACTATGG TATGACTTGA GAAGAAGGAG |
| 2101 | GATCCGTGCT TAAGTACATC CTAA |

The PSORT algorithm predicts inner membrane (0.162).

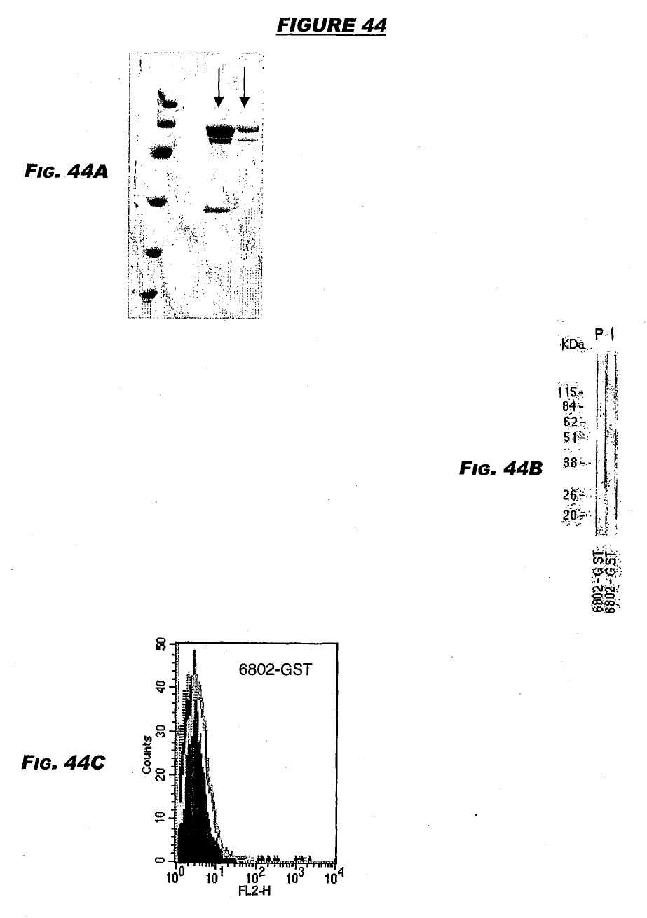

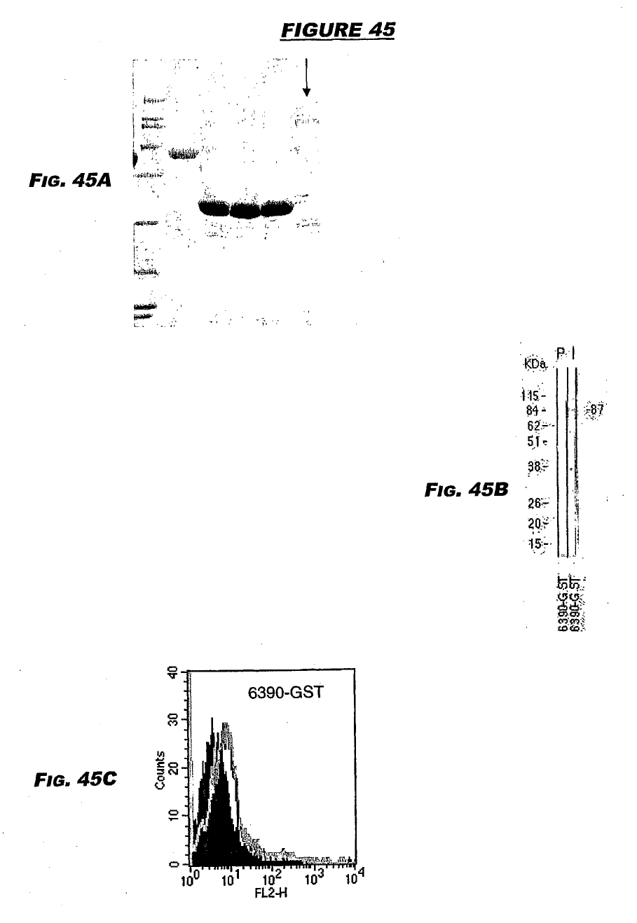

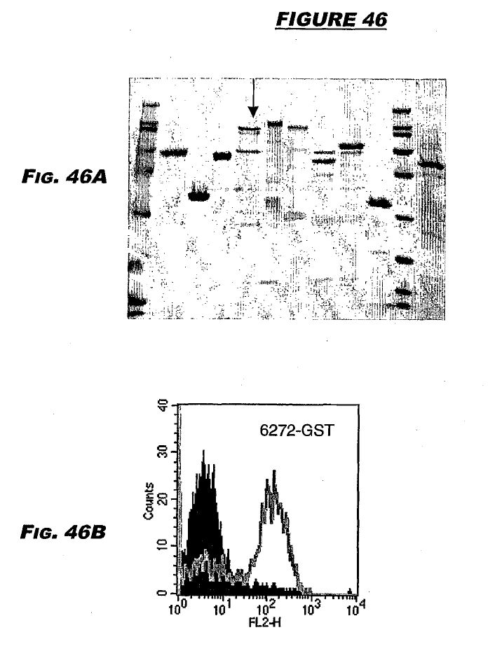

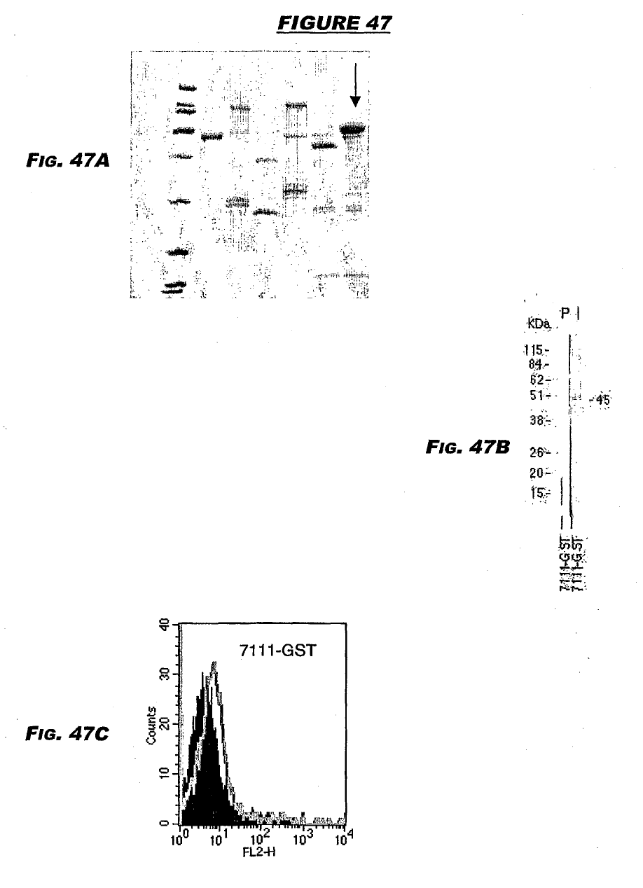

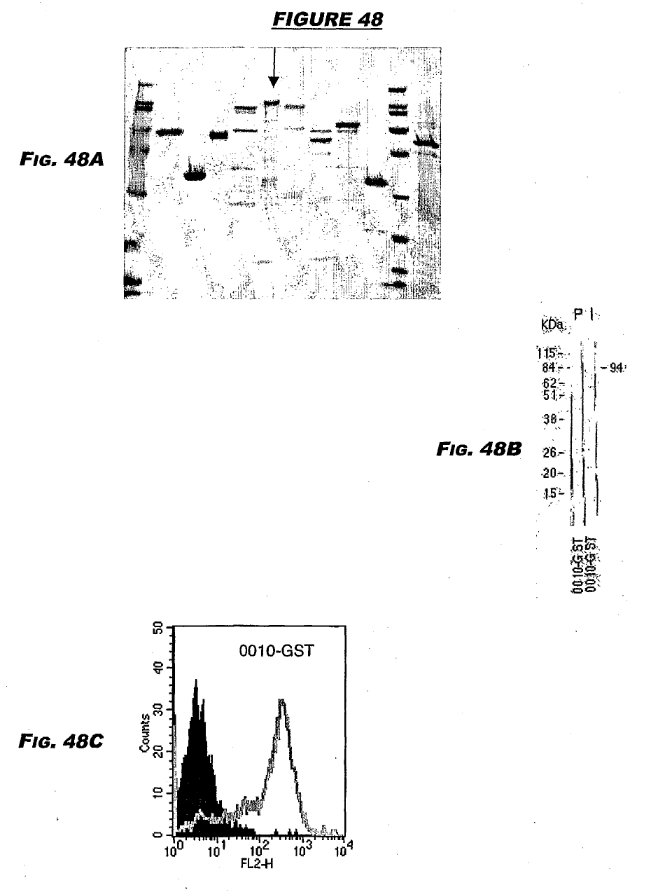

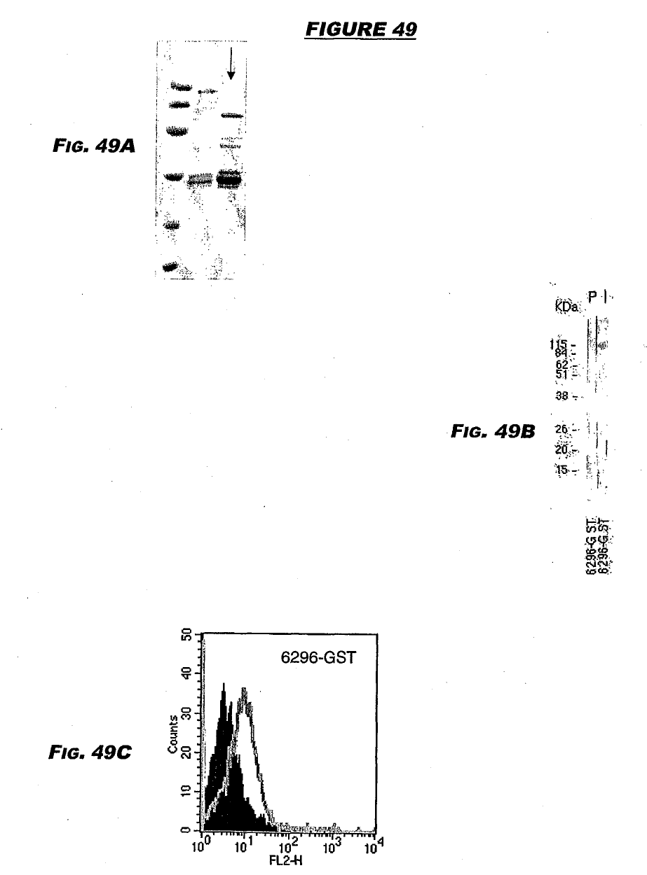

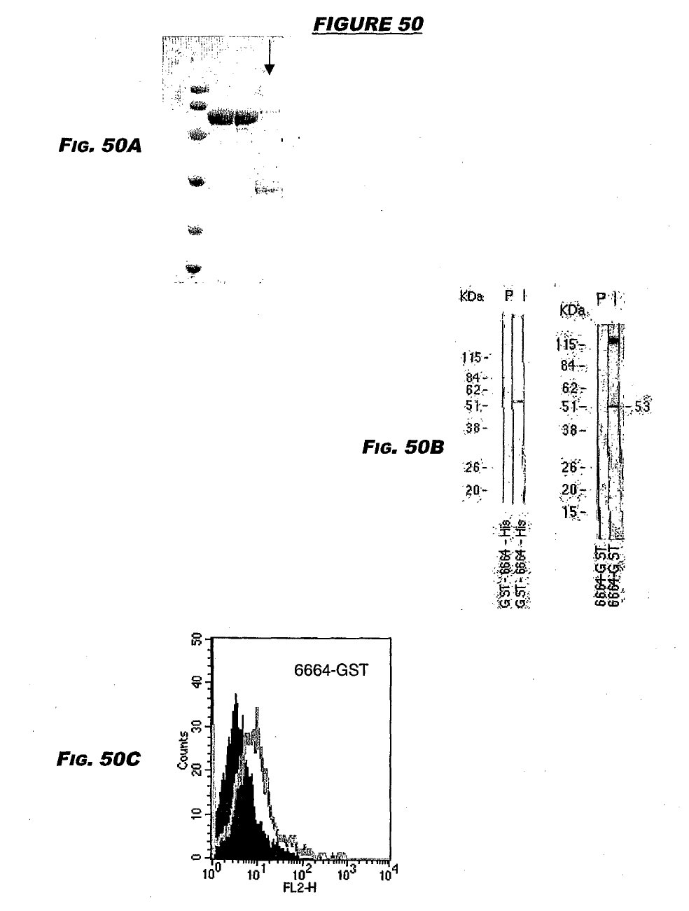

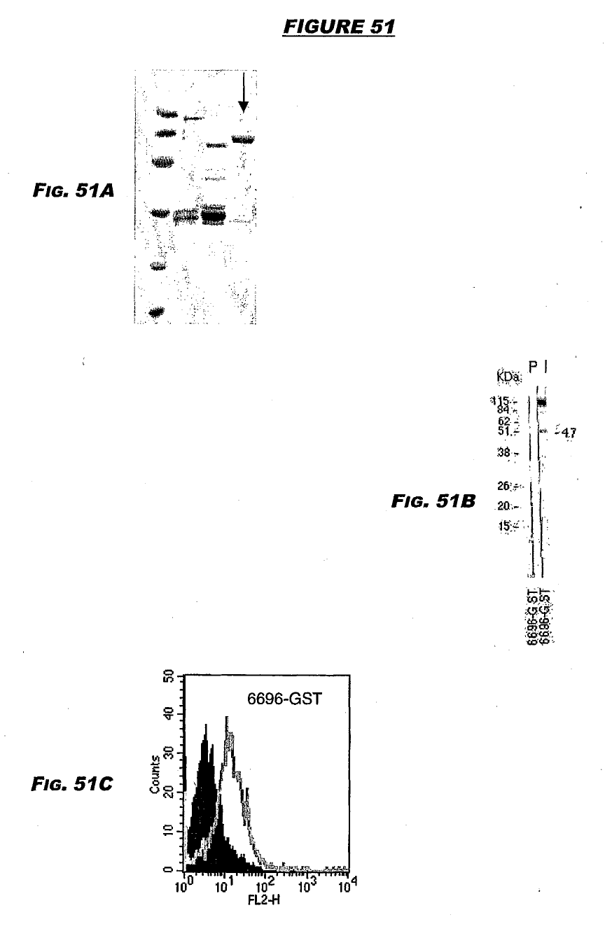

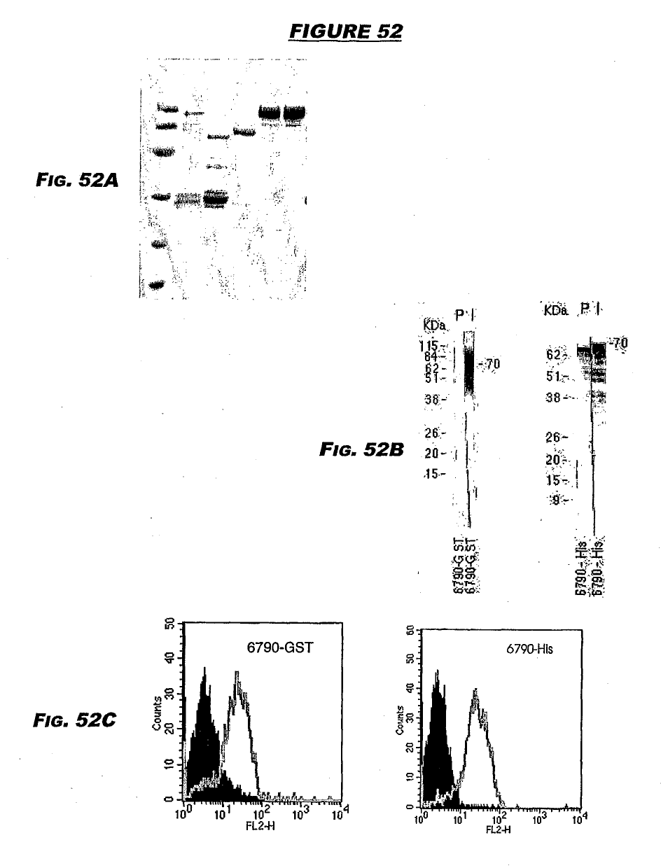

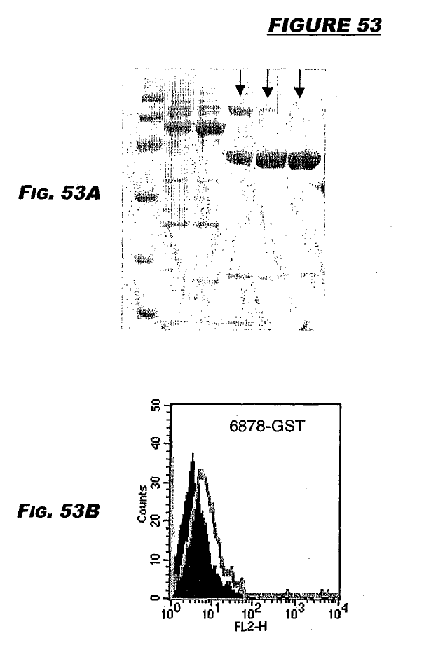

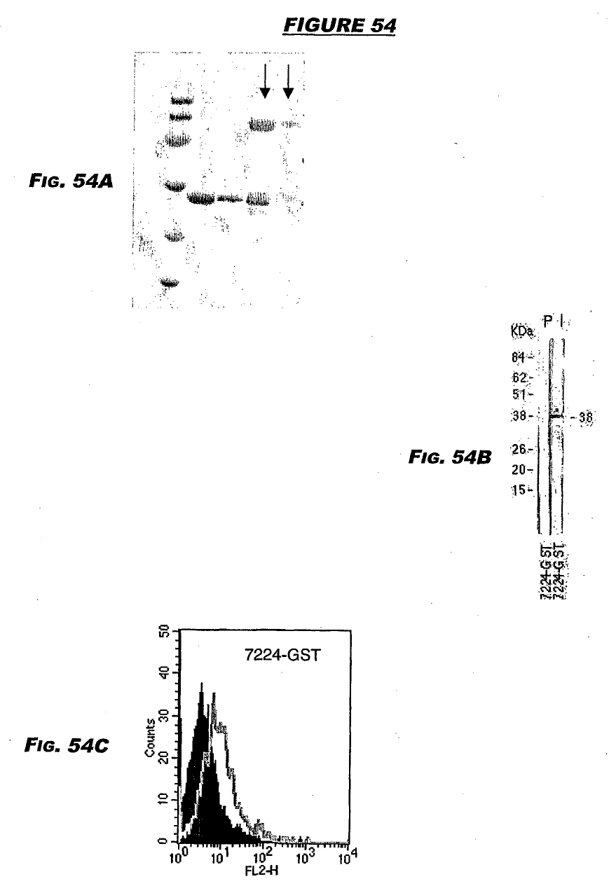

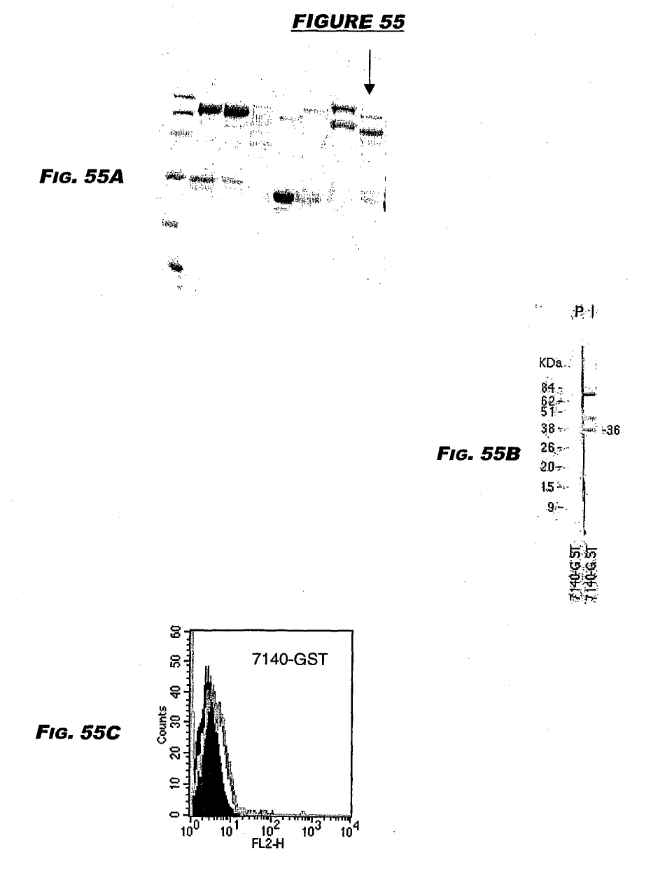

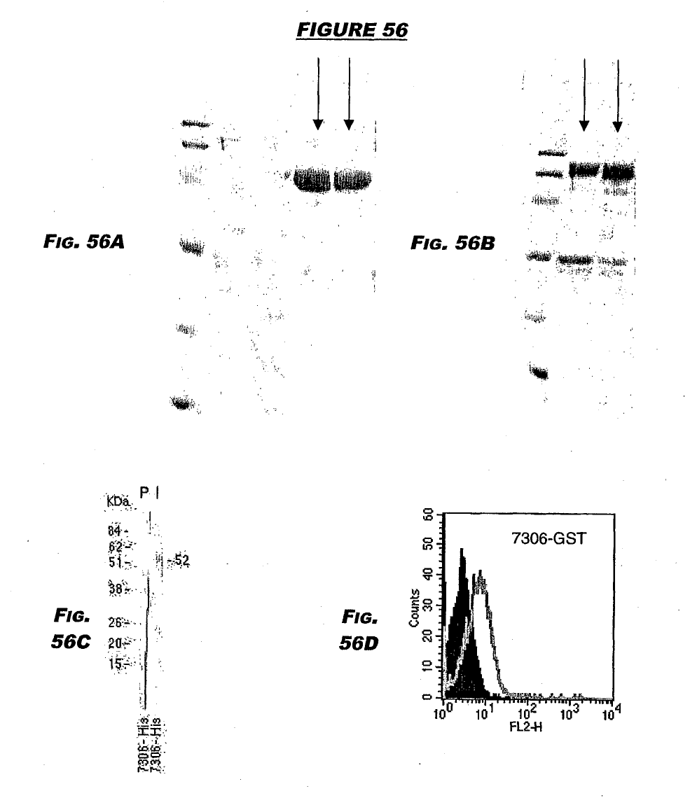

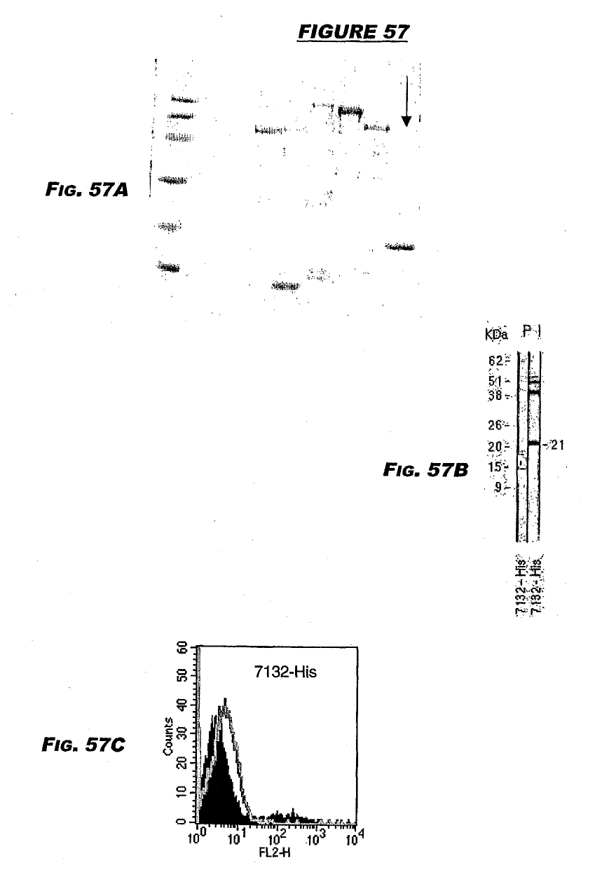

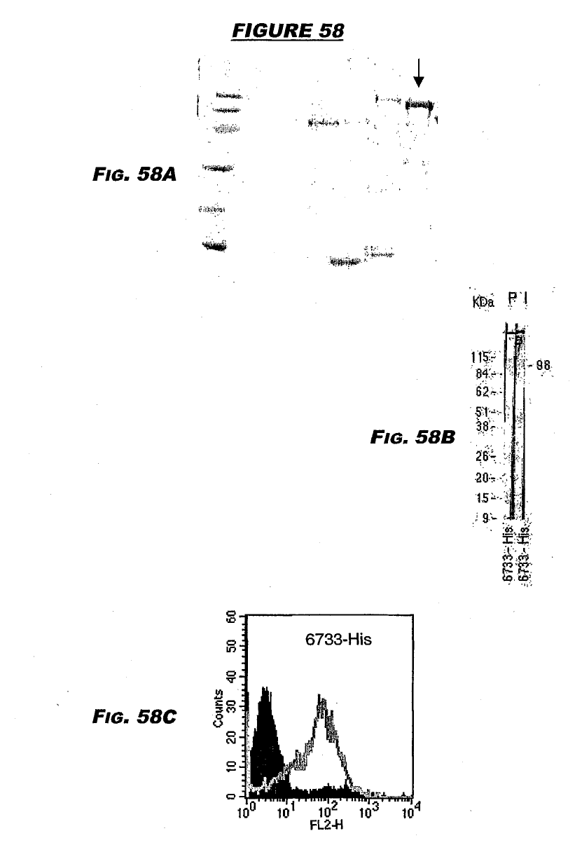

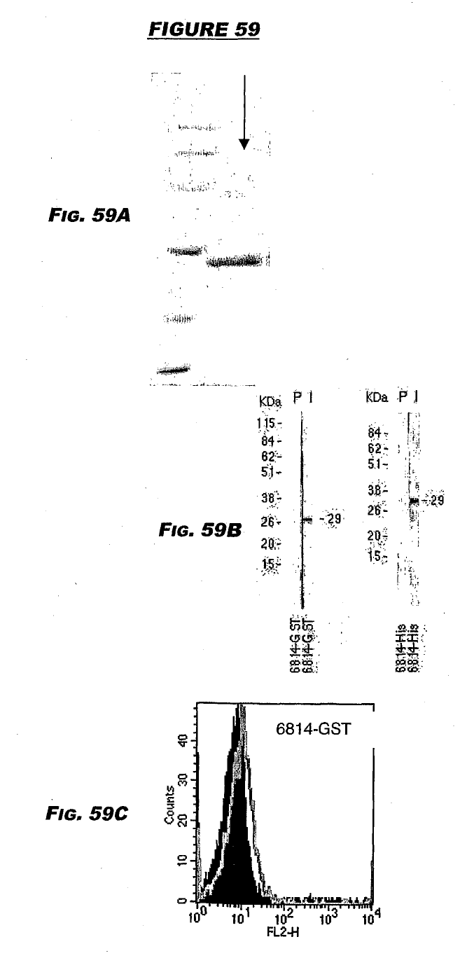

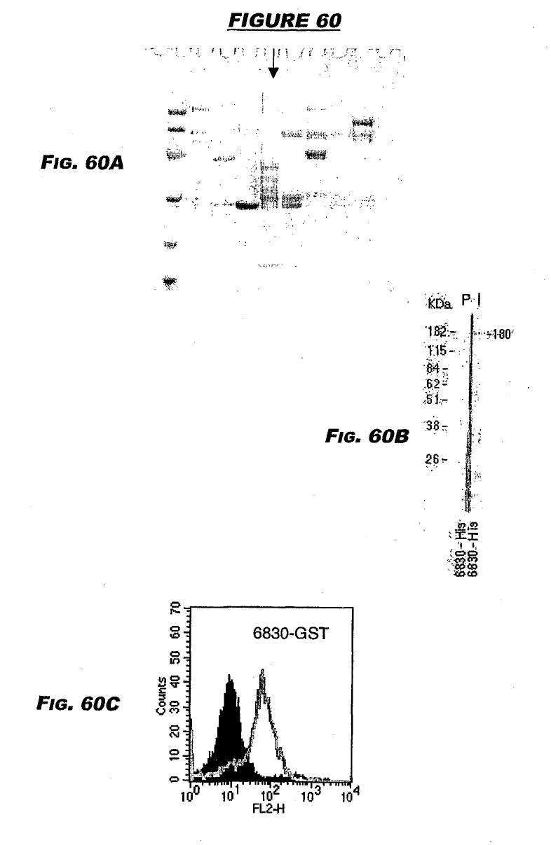

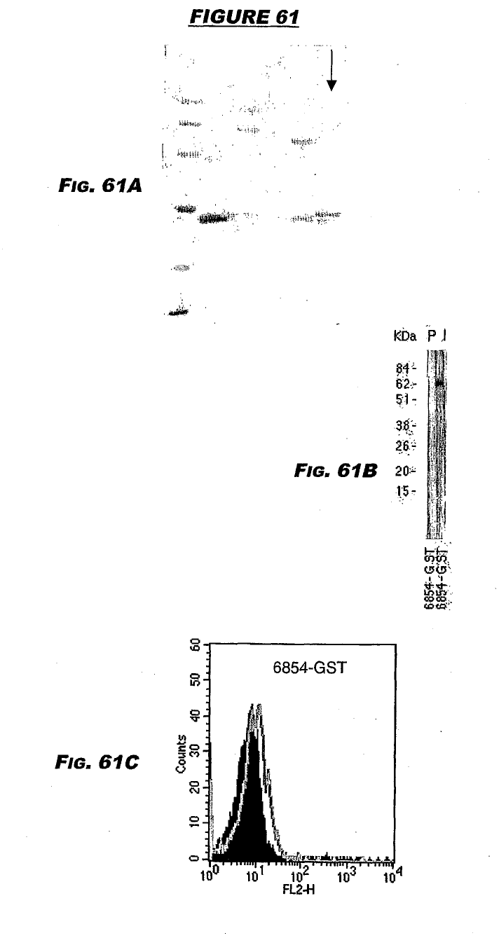

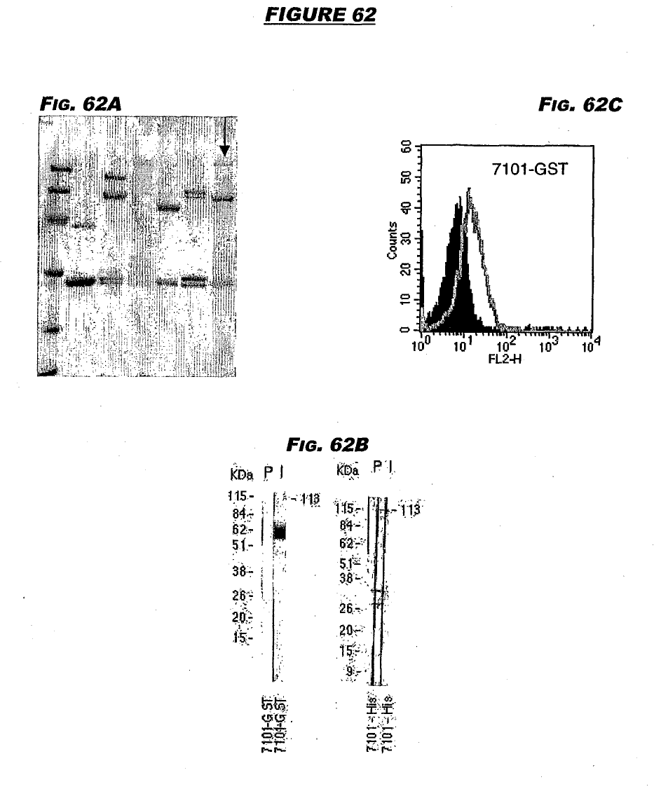

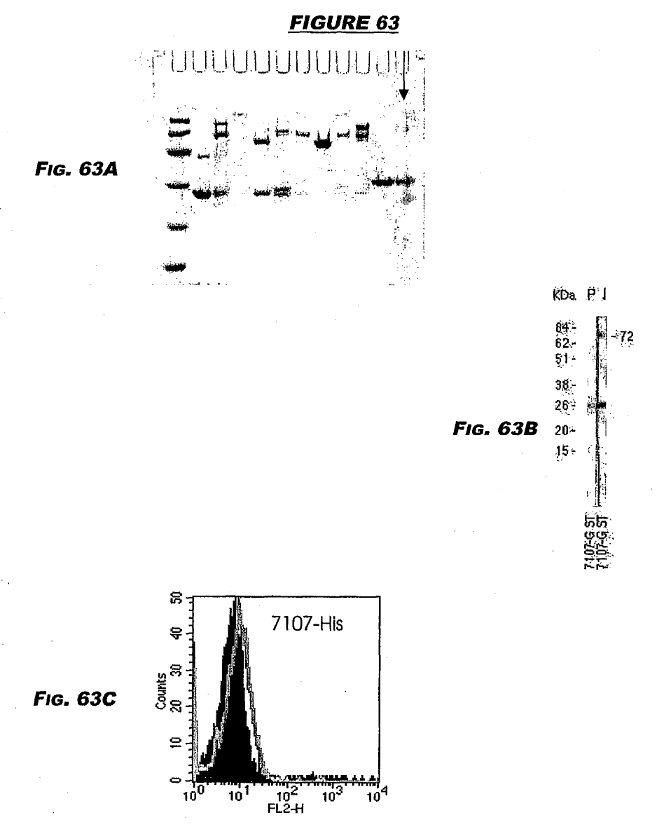

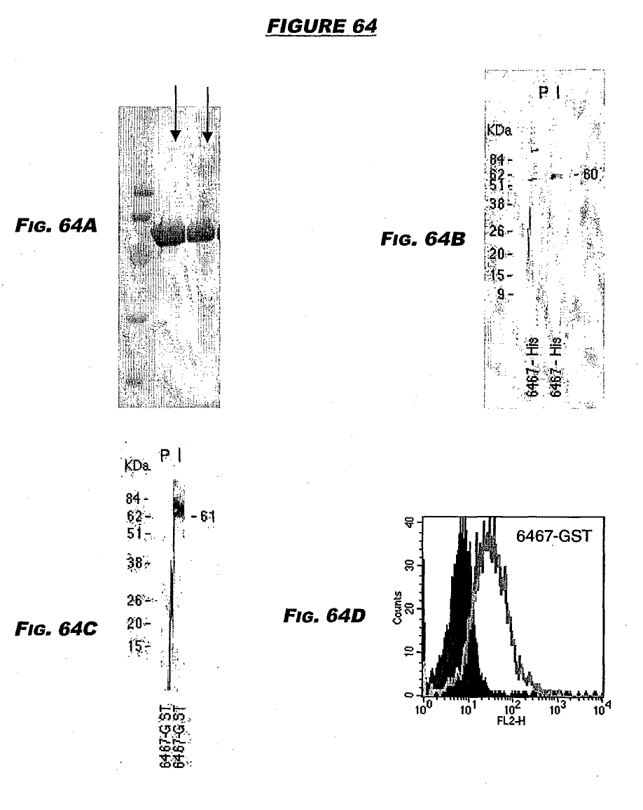

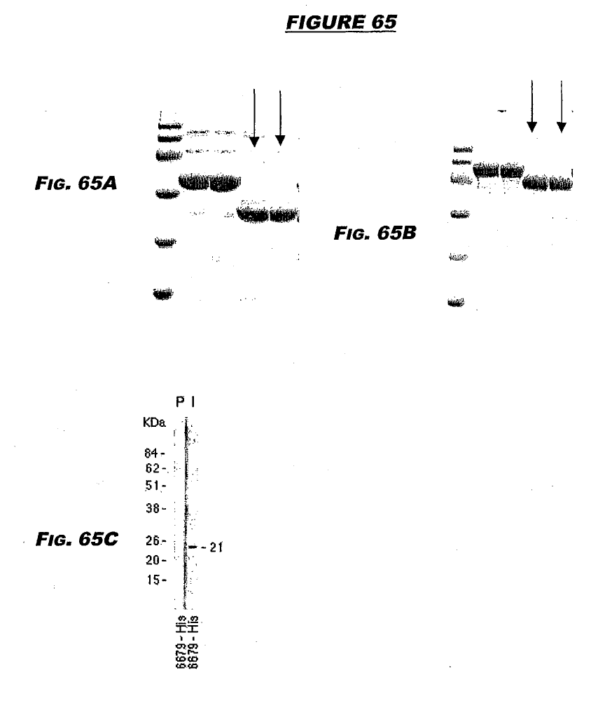

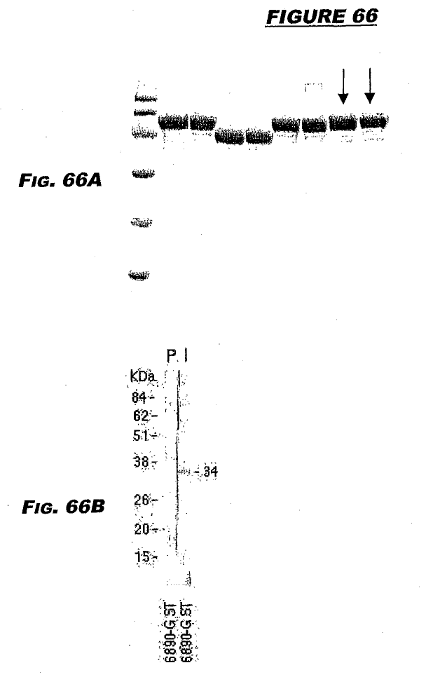

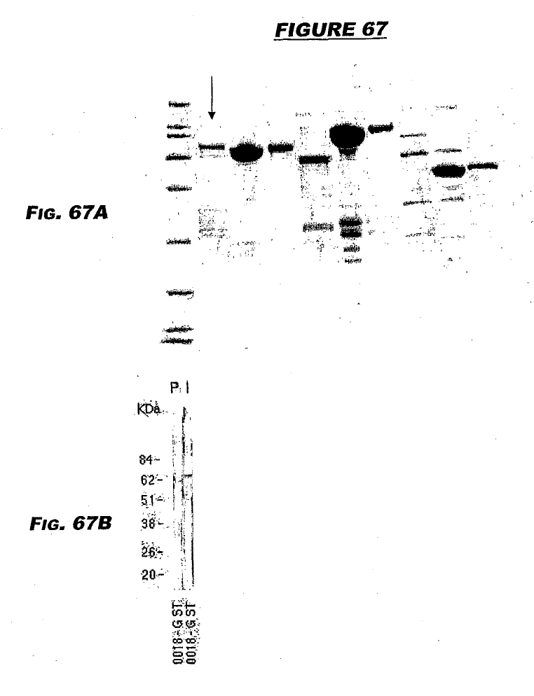

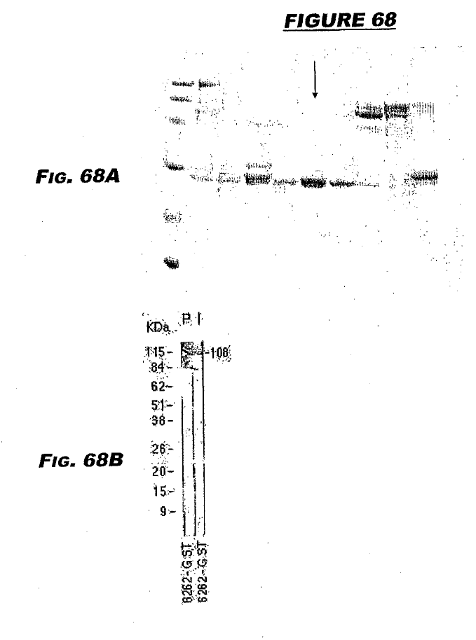

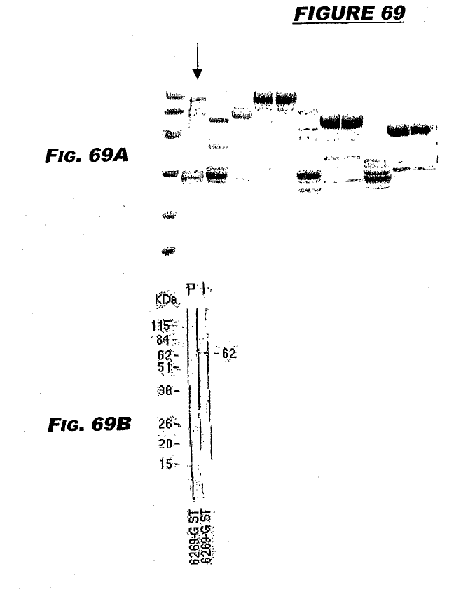

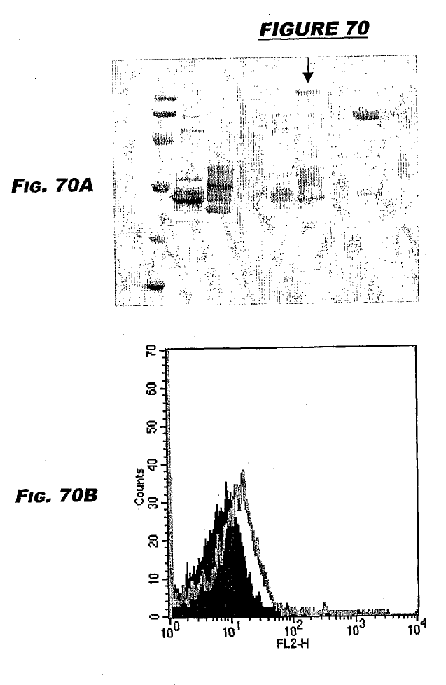

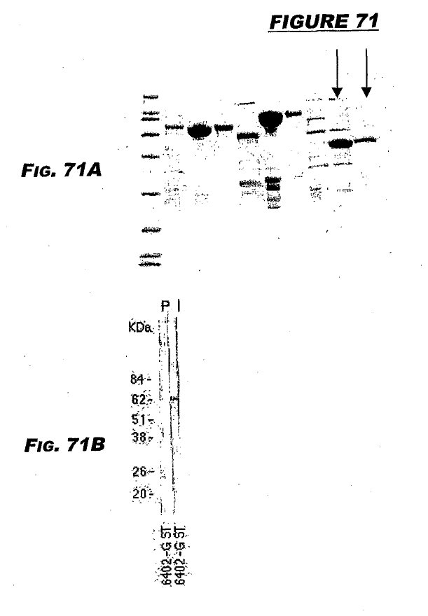

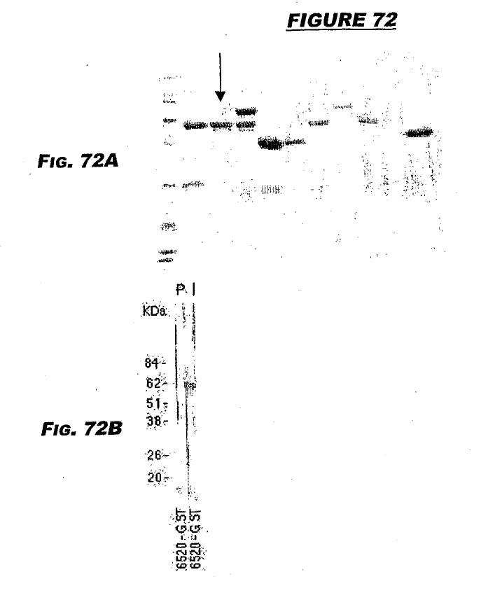

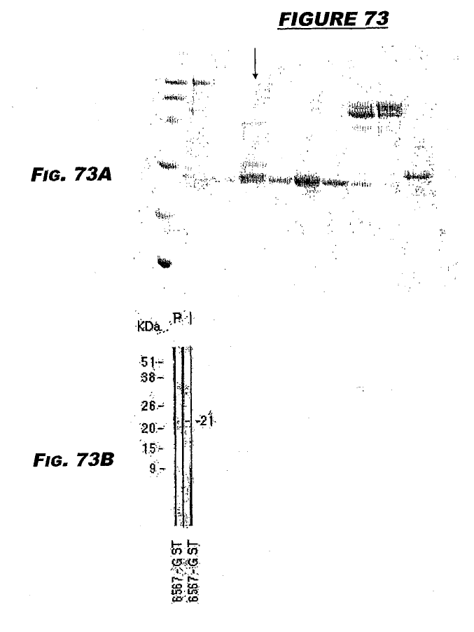

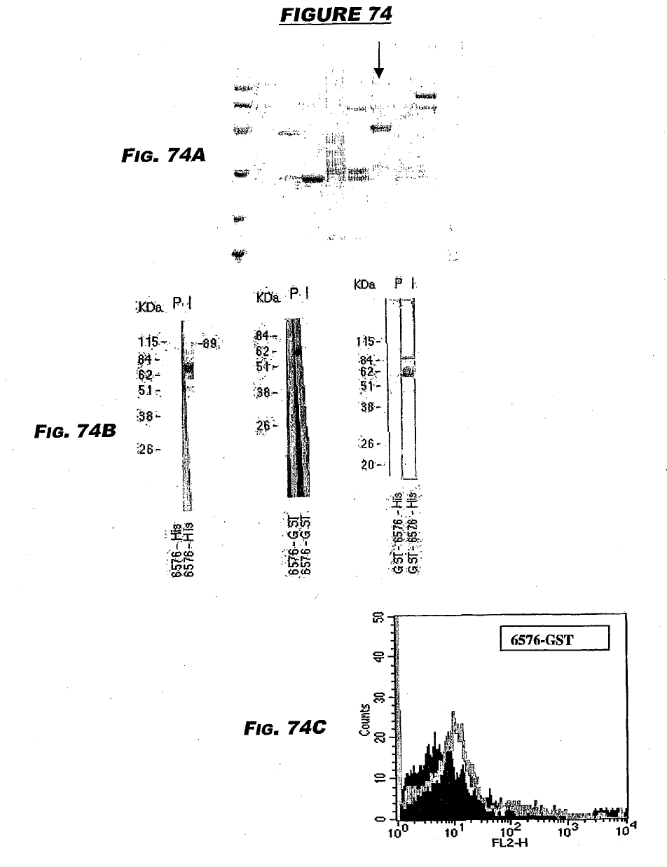

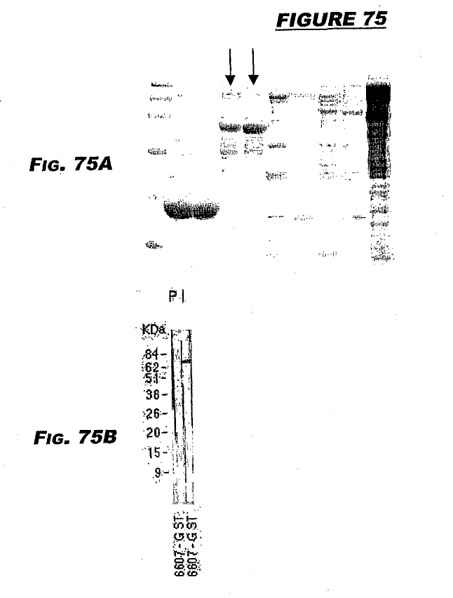

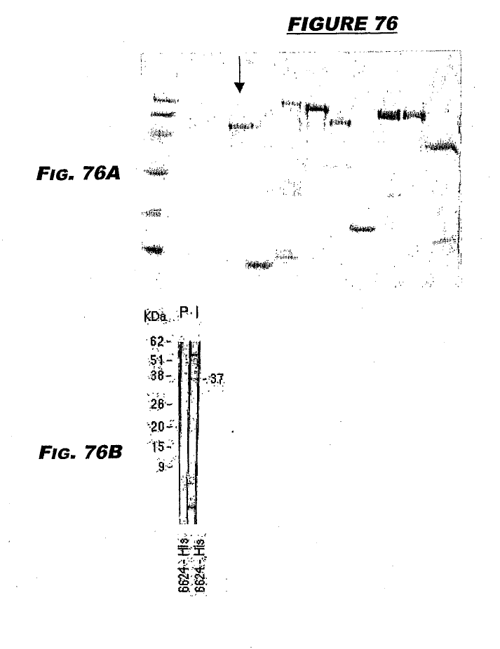

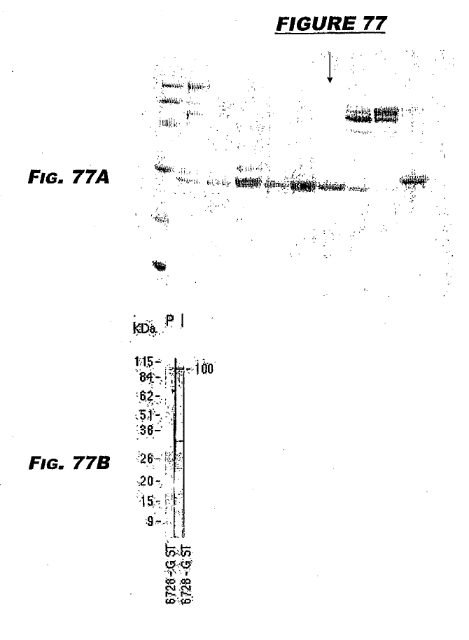

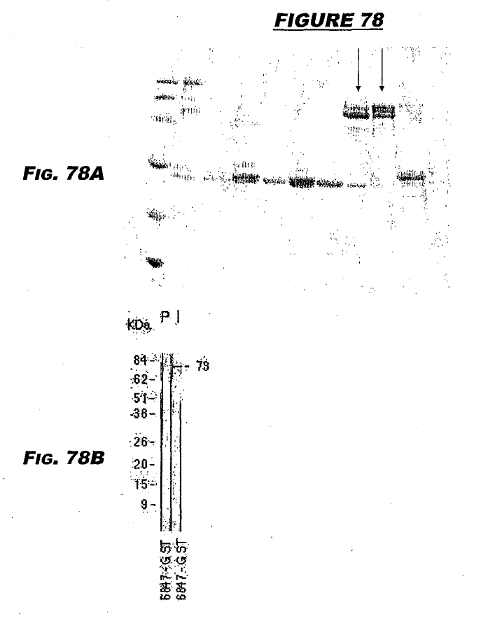

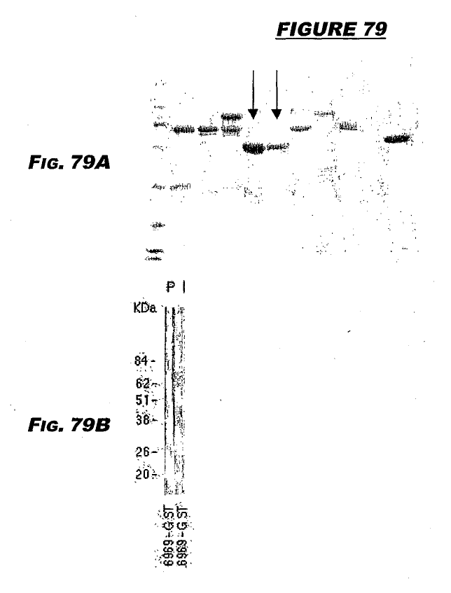

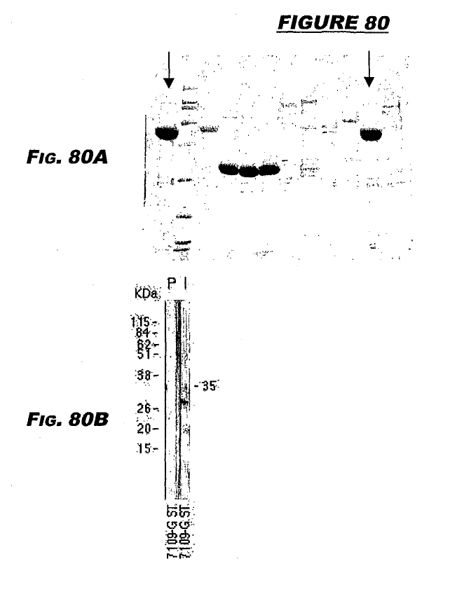

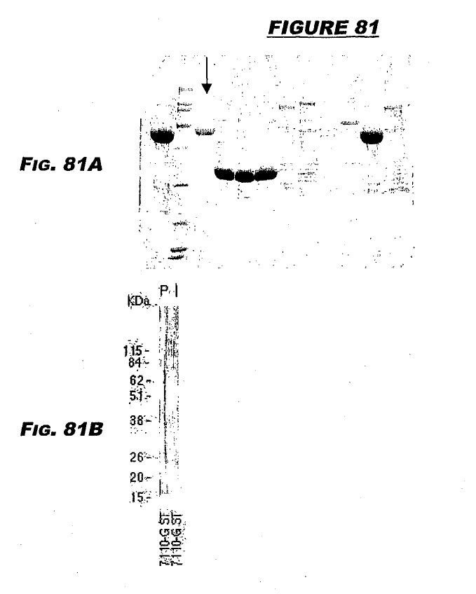

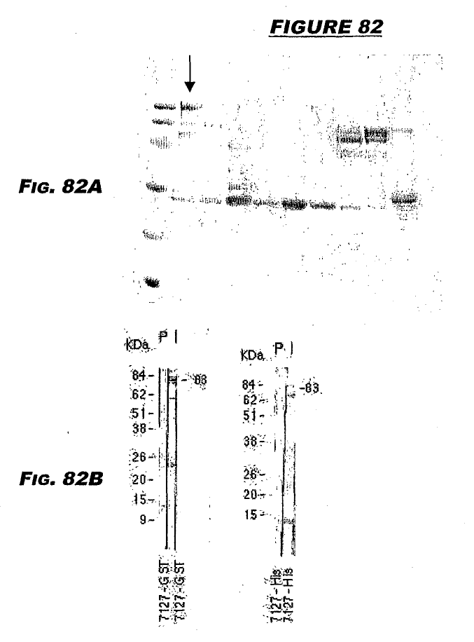

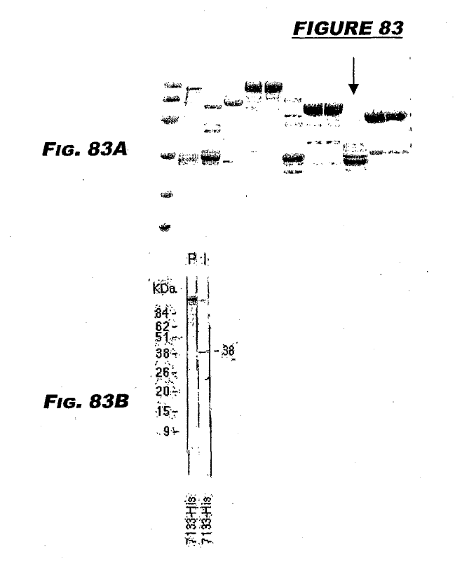

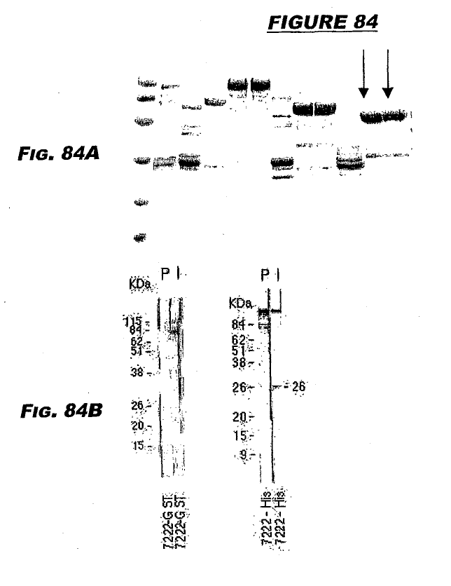

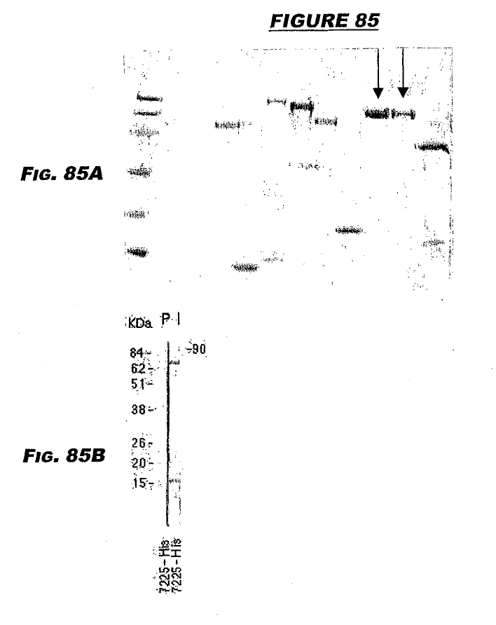

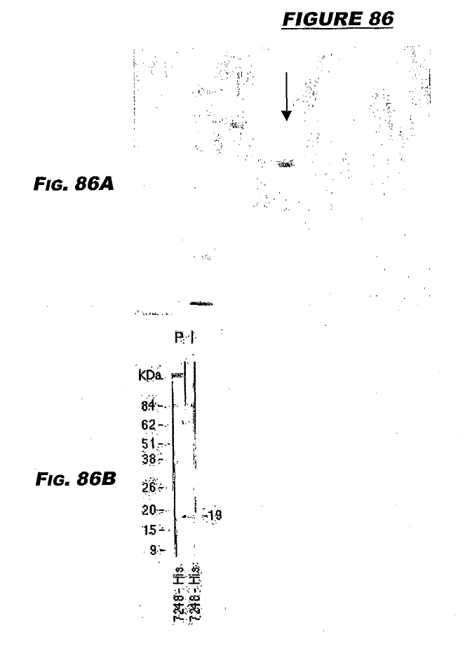

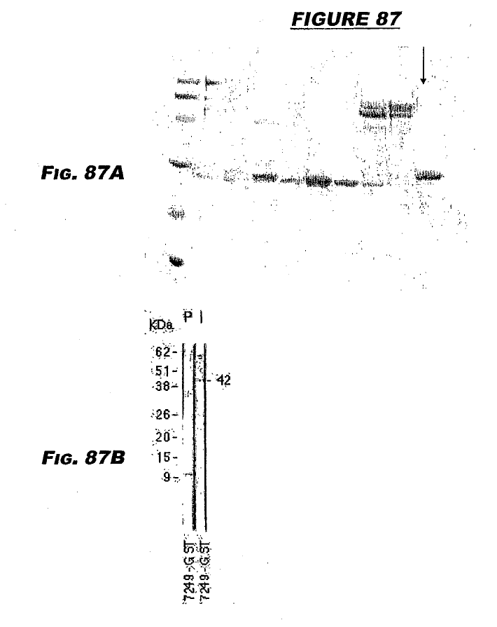

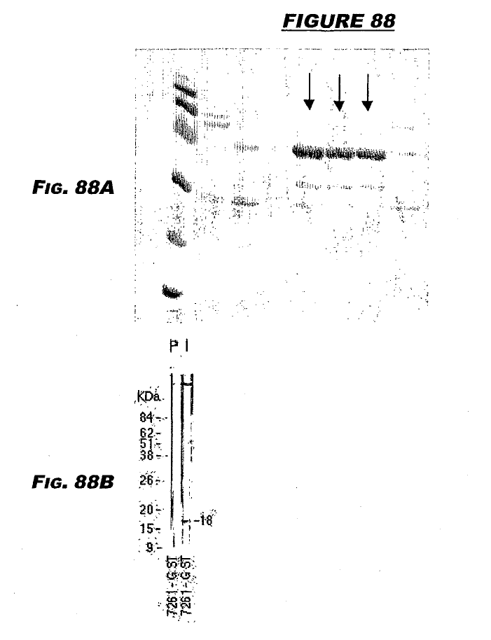

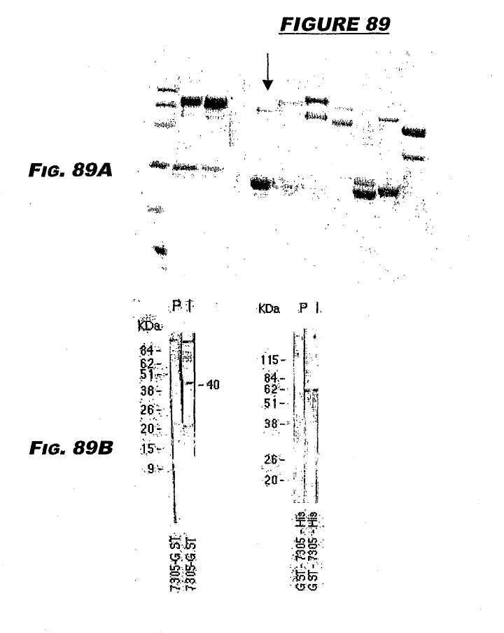

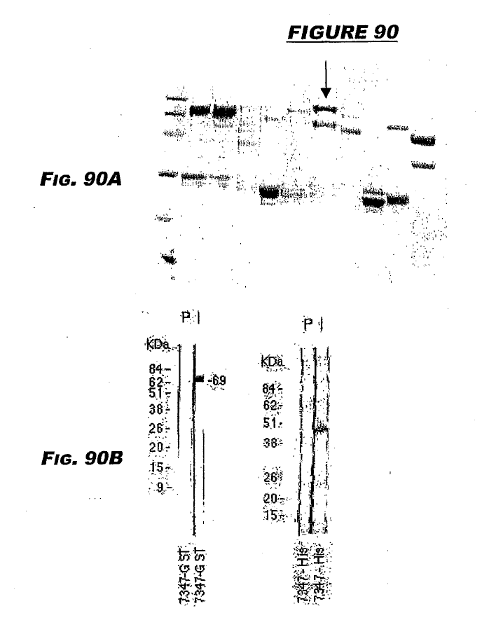

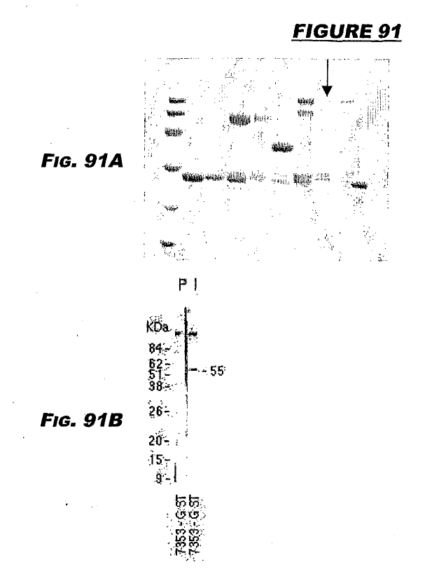

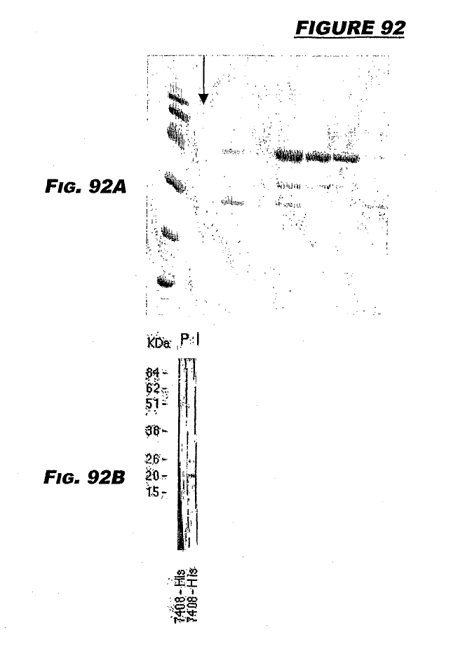

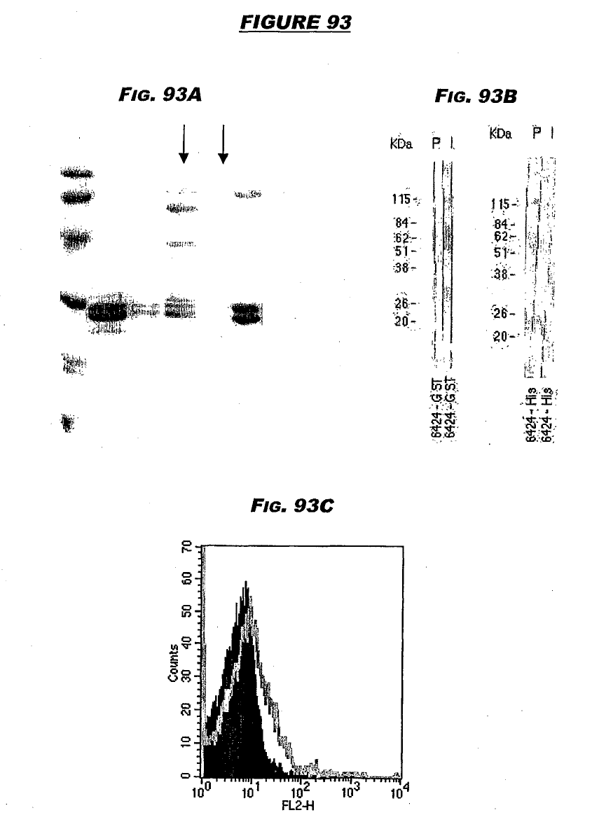

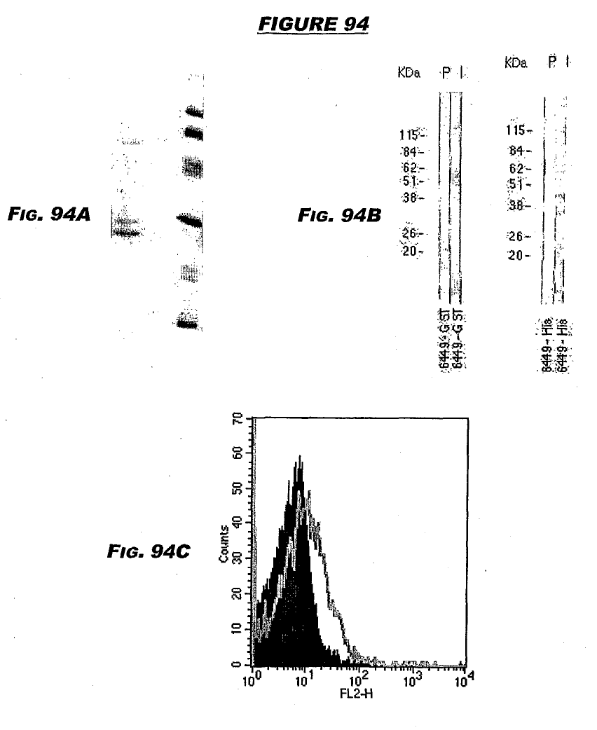

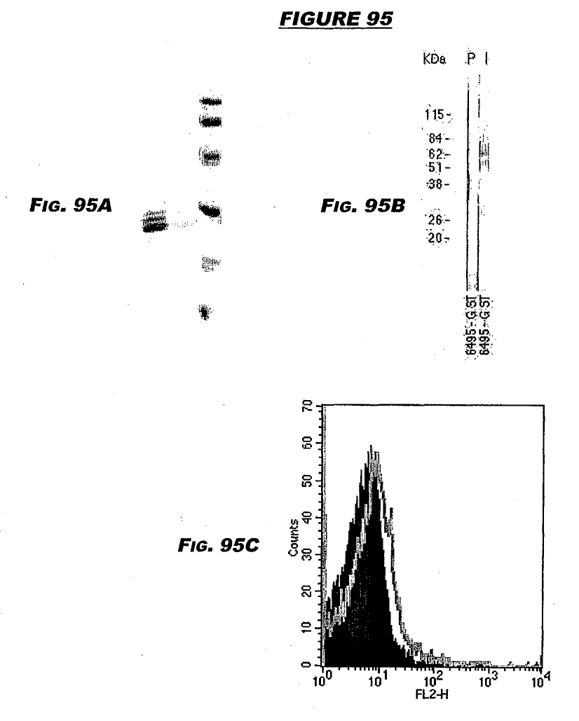

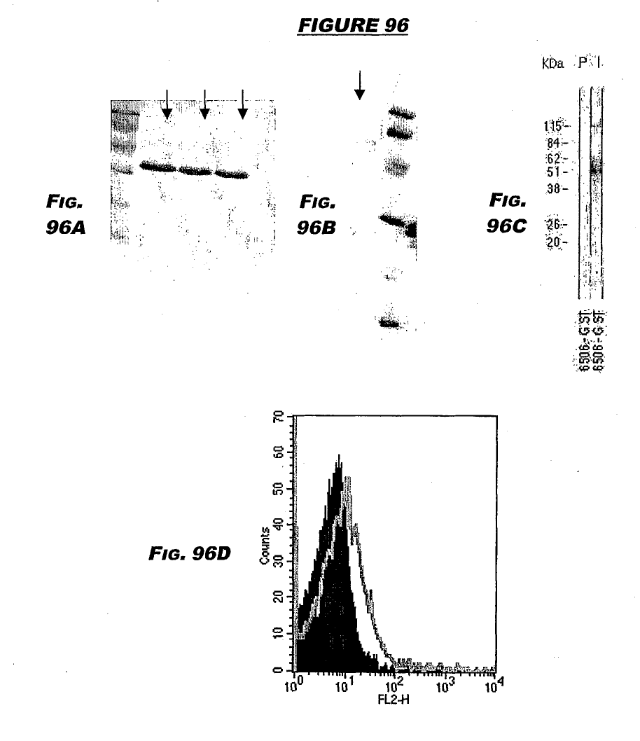

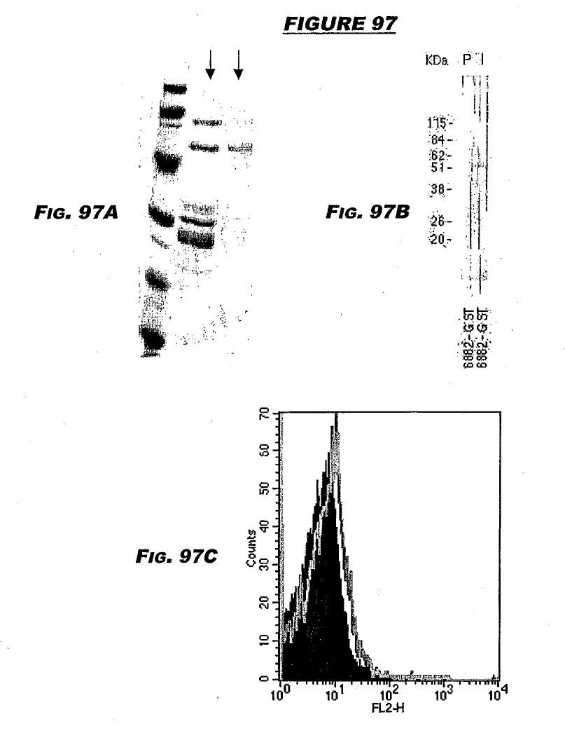

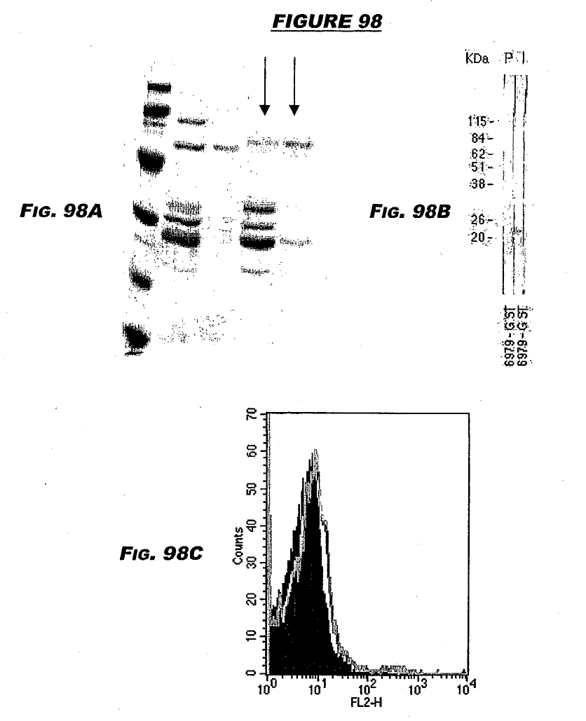

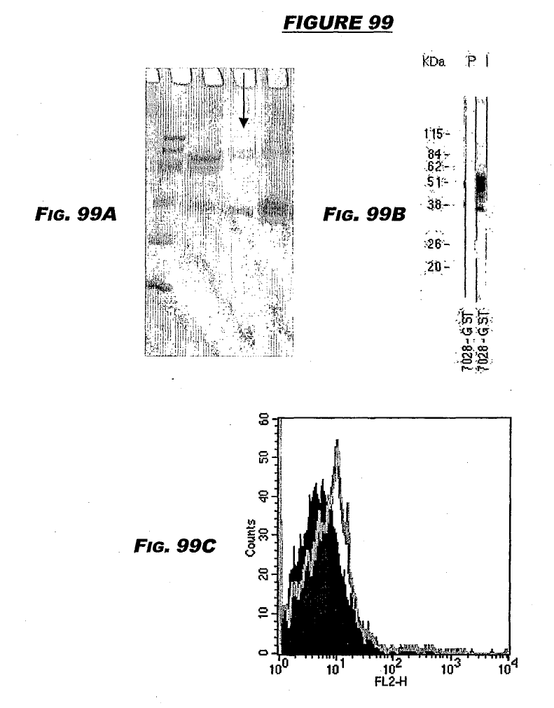

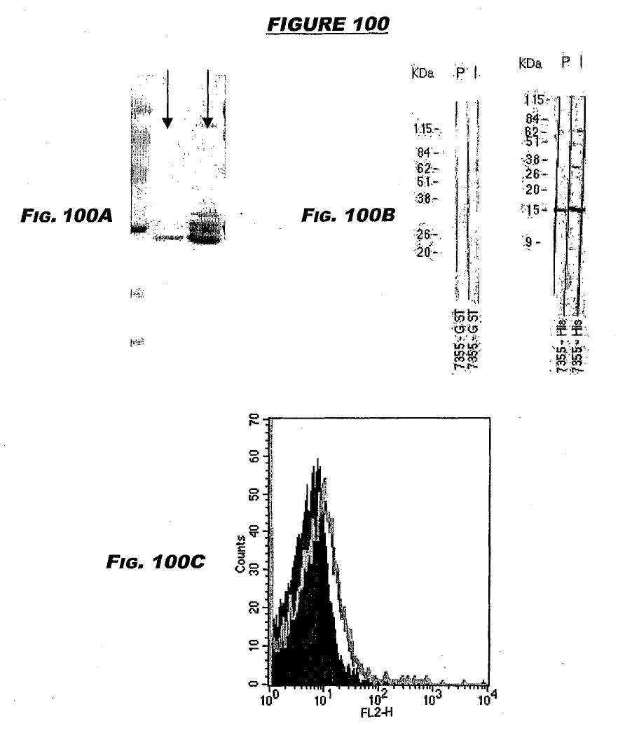

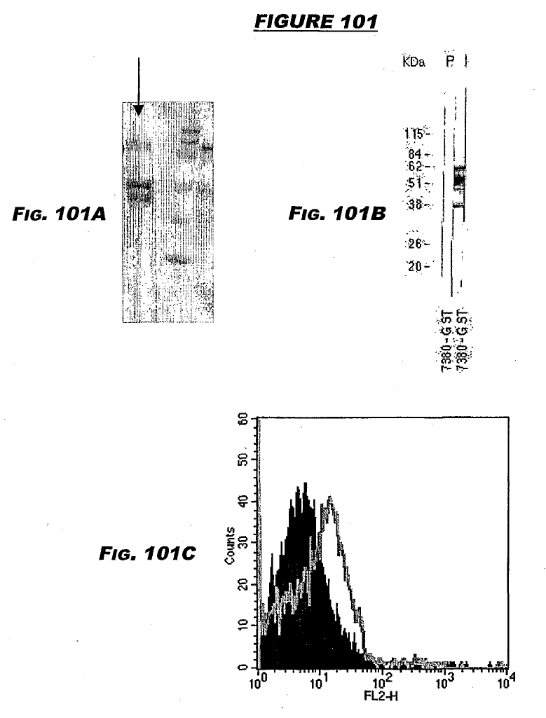

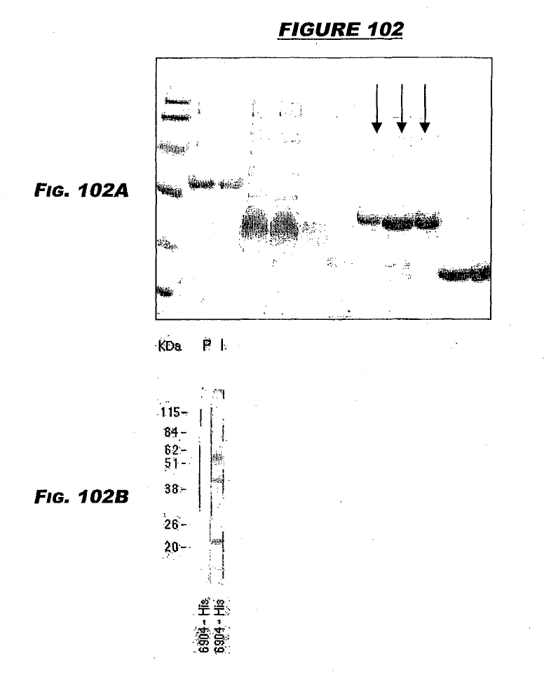

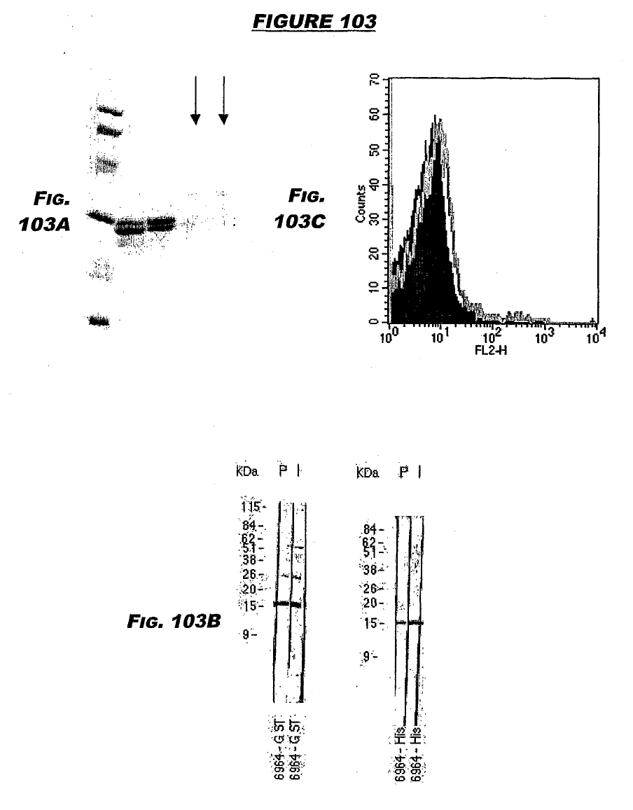

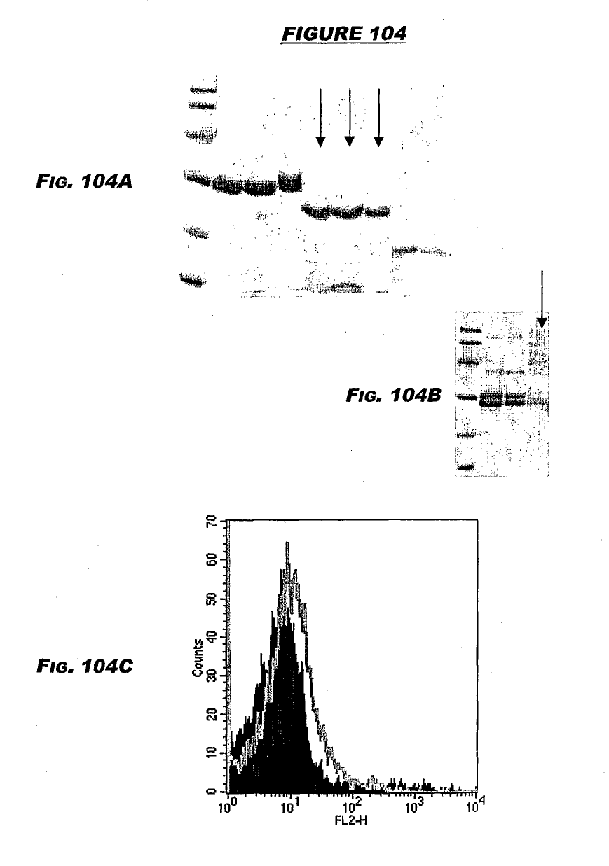

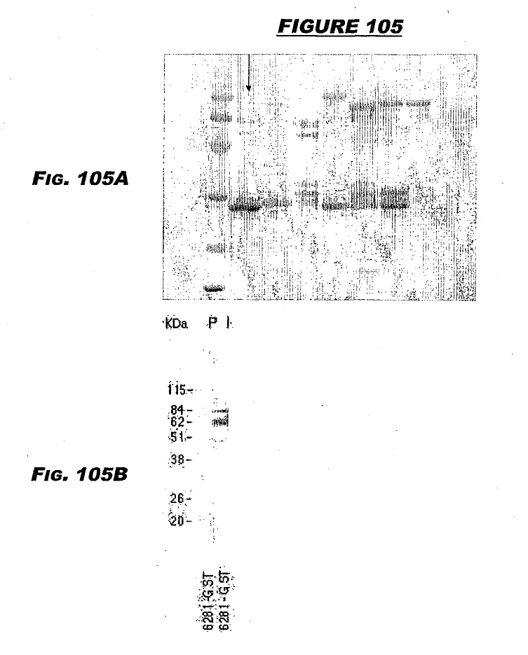

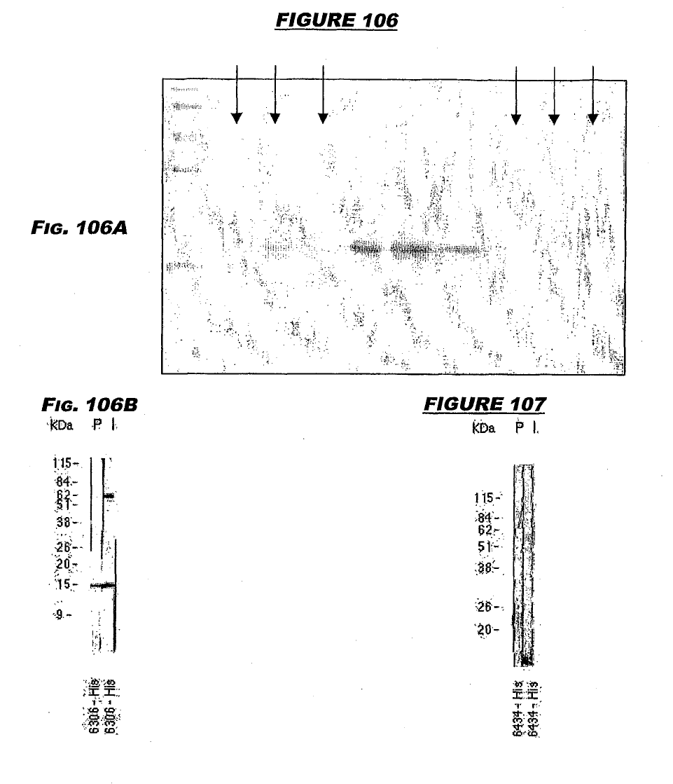

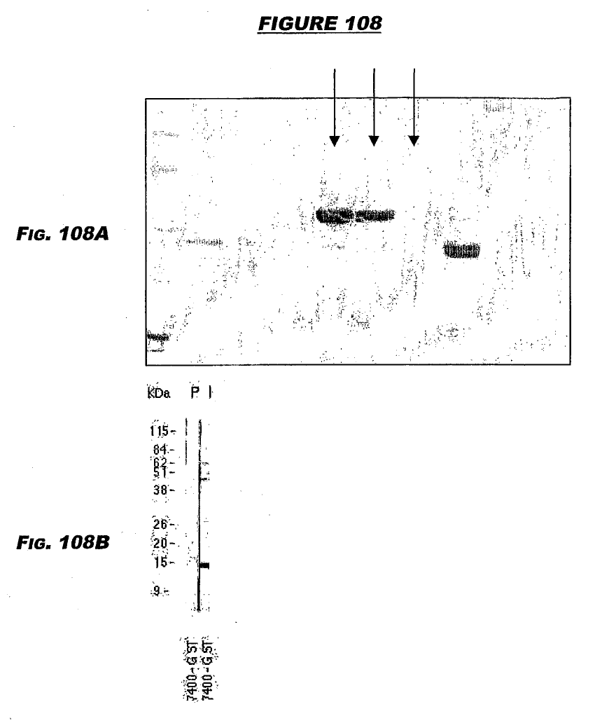

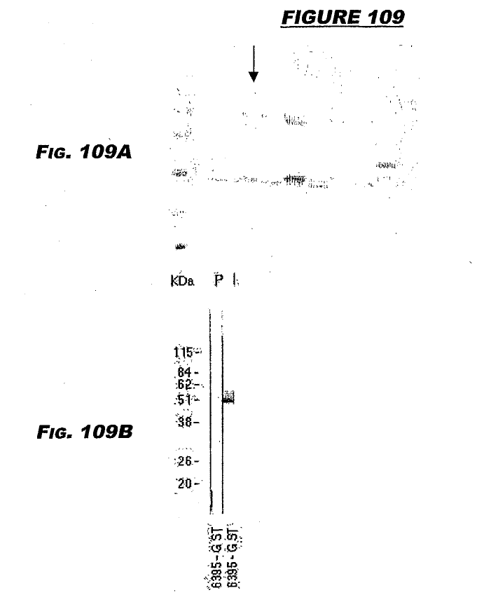

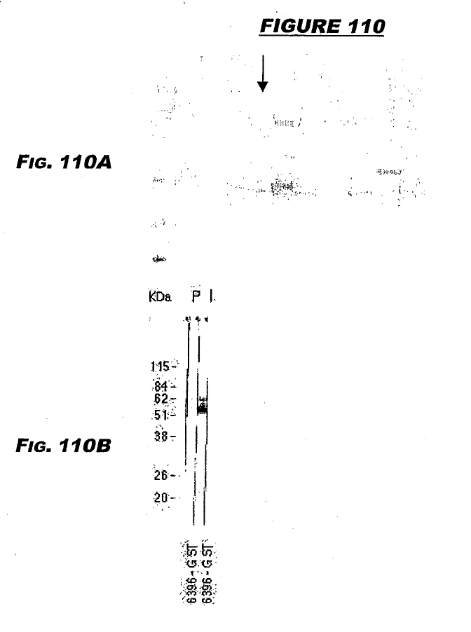

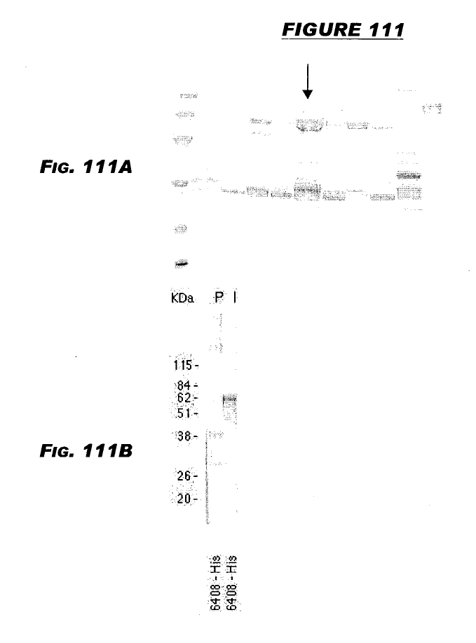

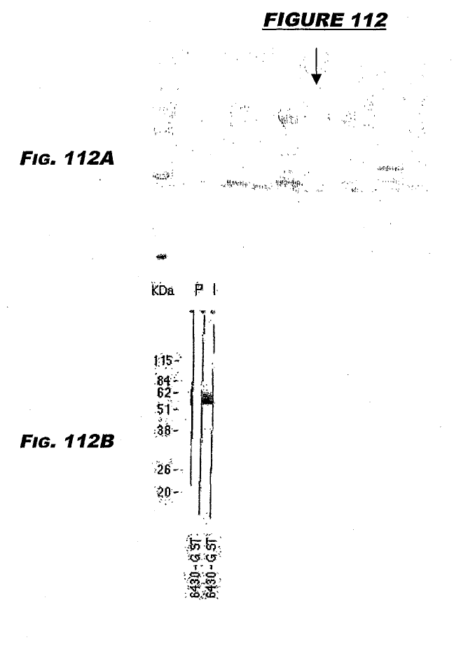

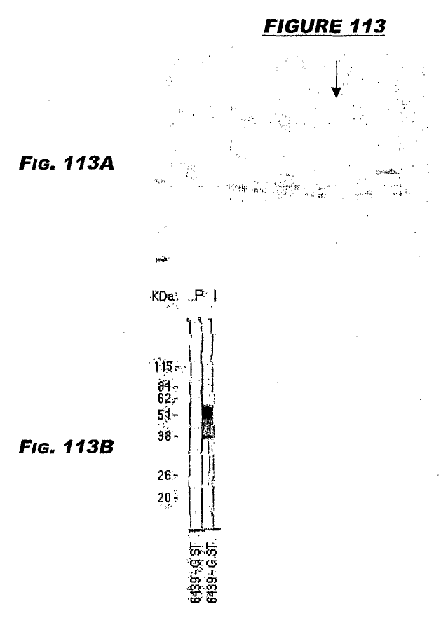

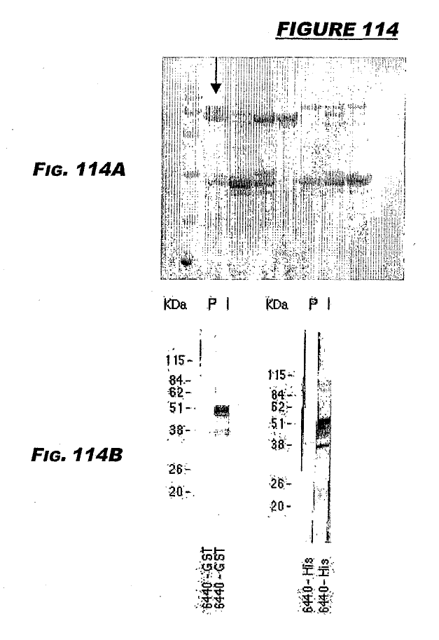

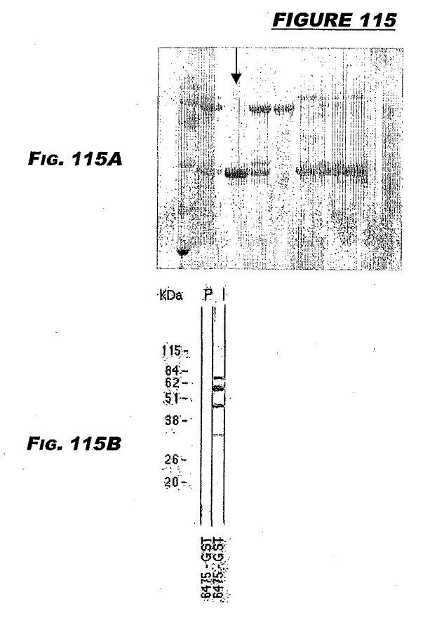

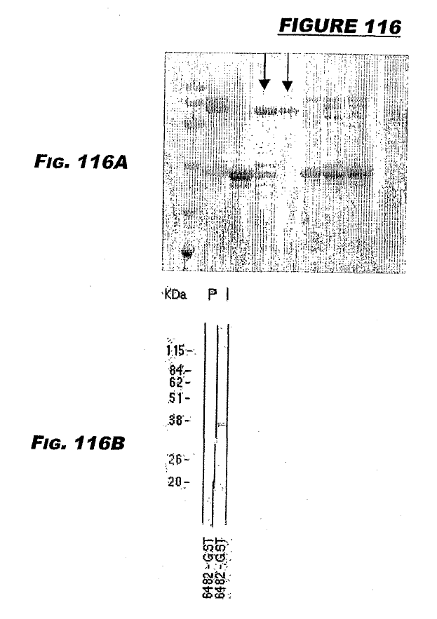

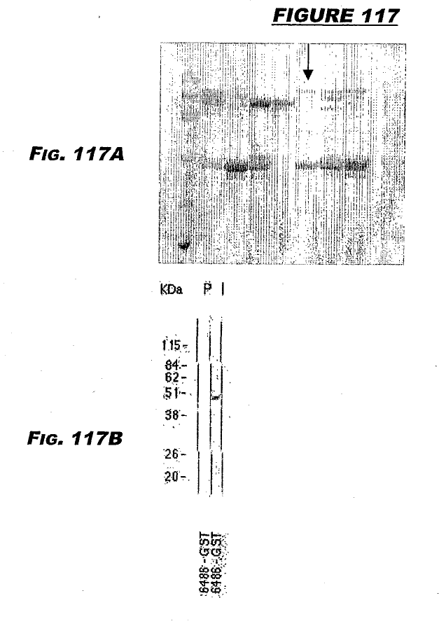

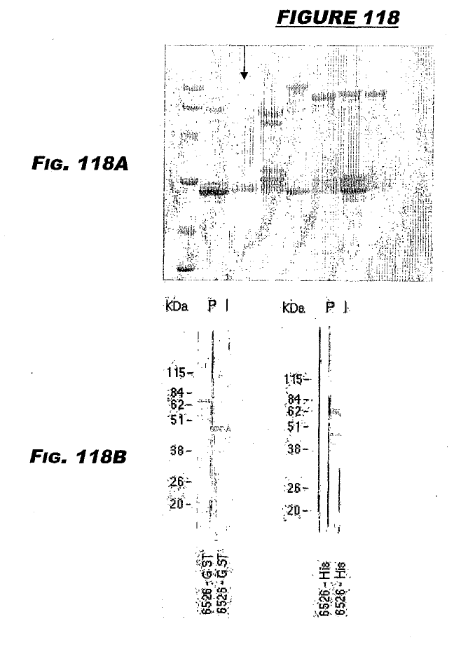

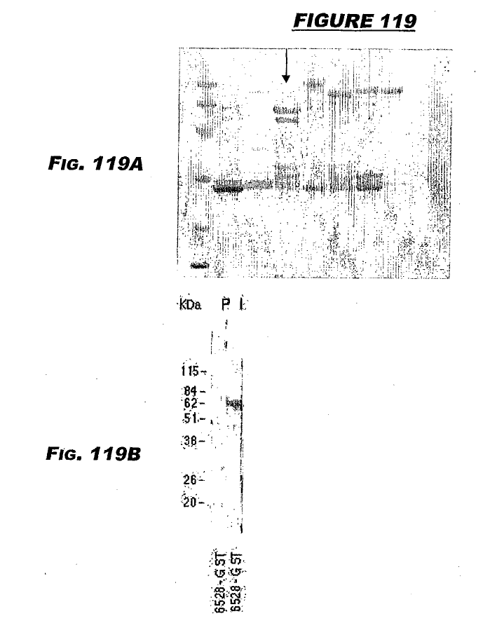

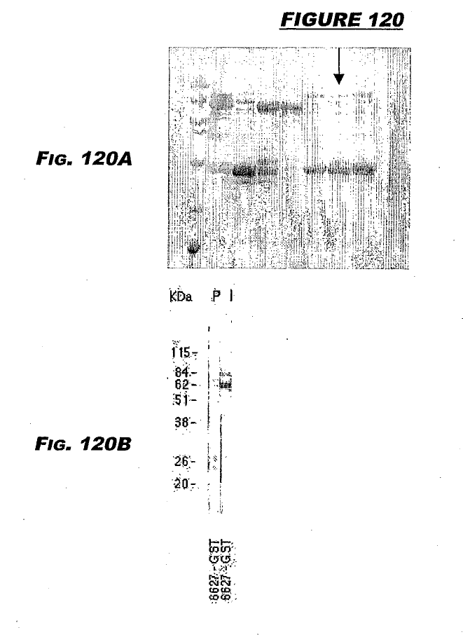

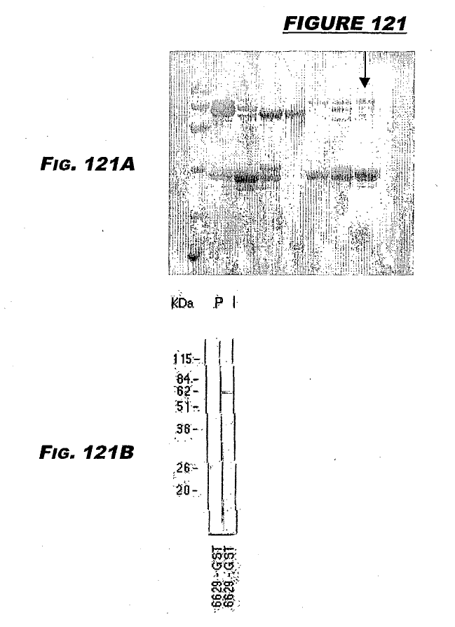

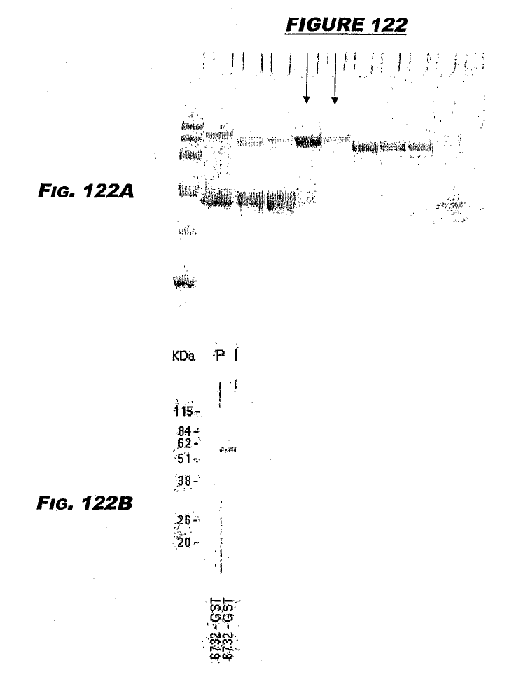

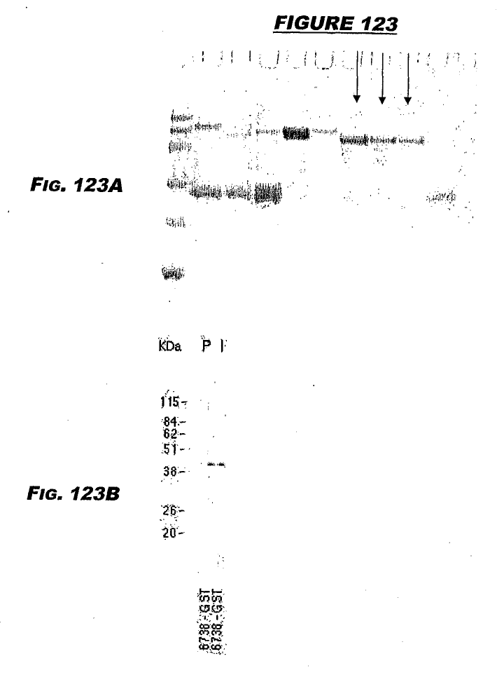

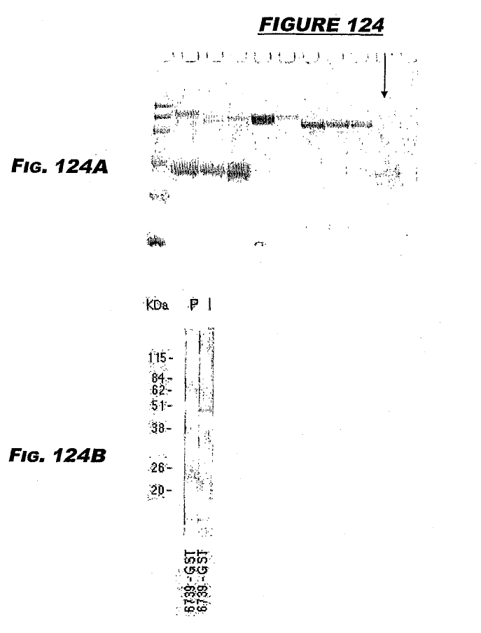

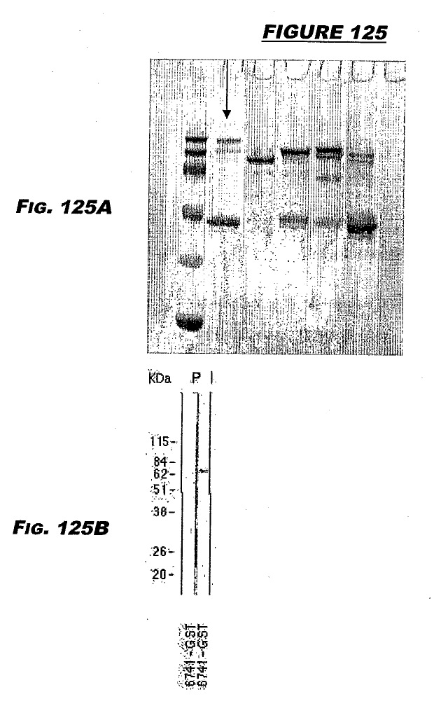

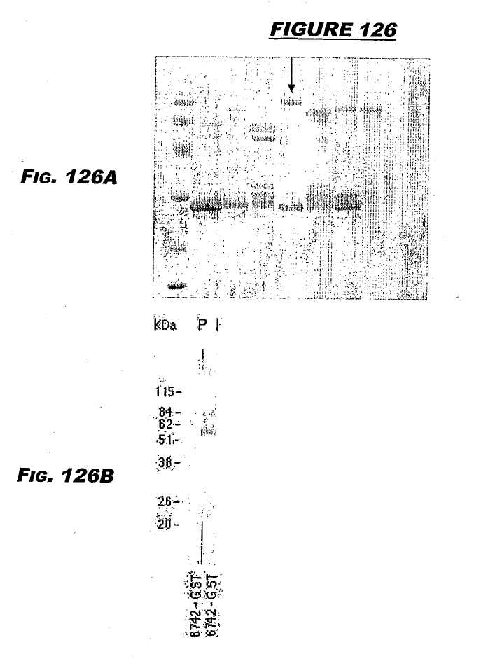

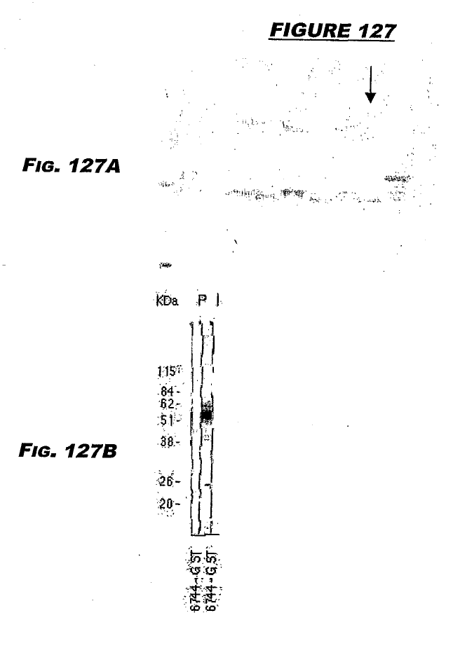

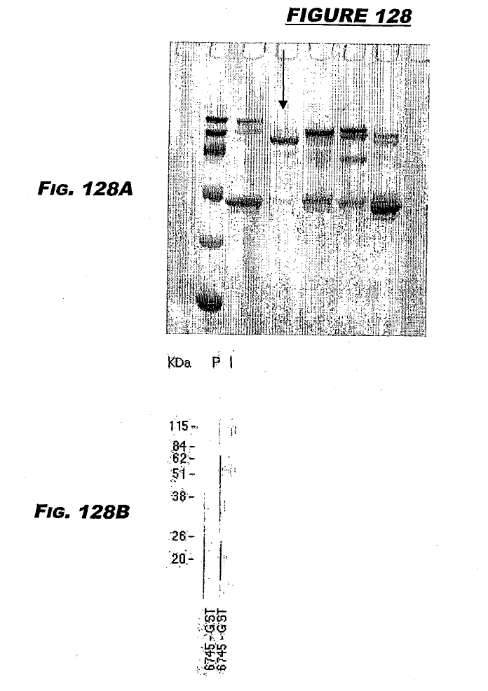

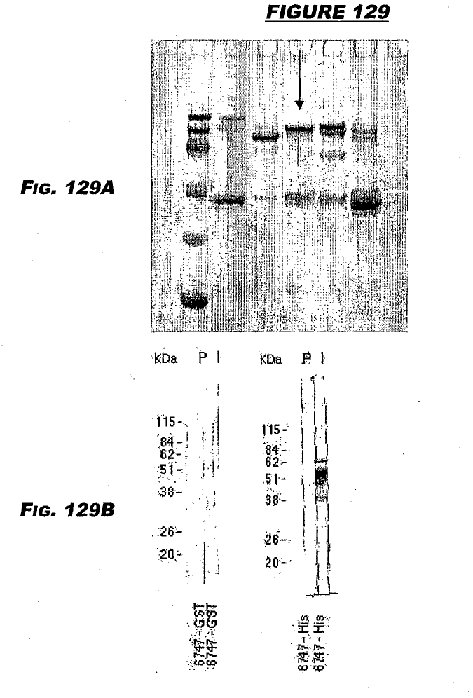

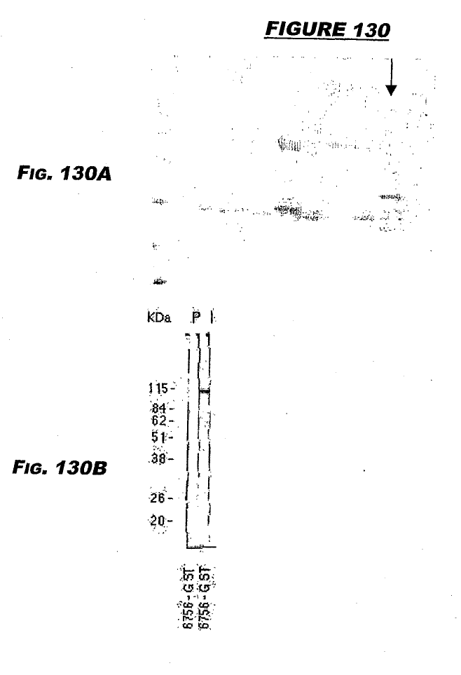

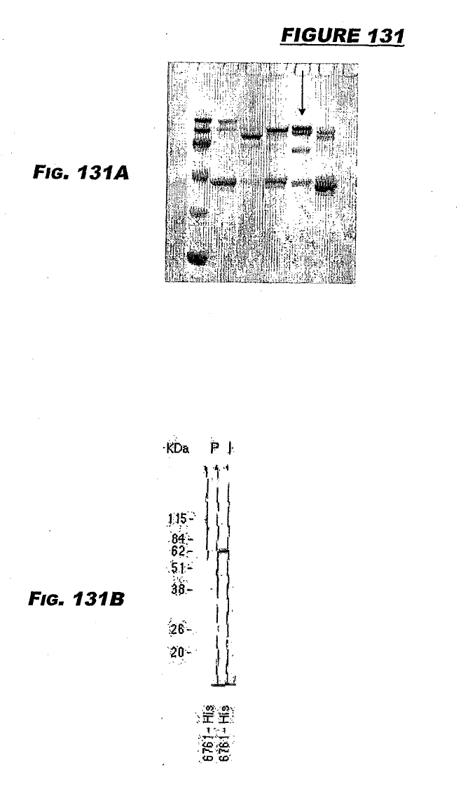

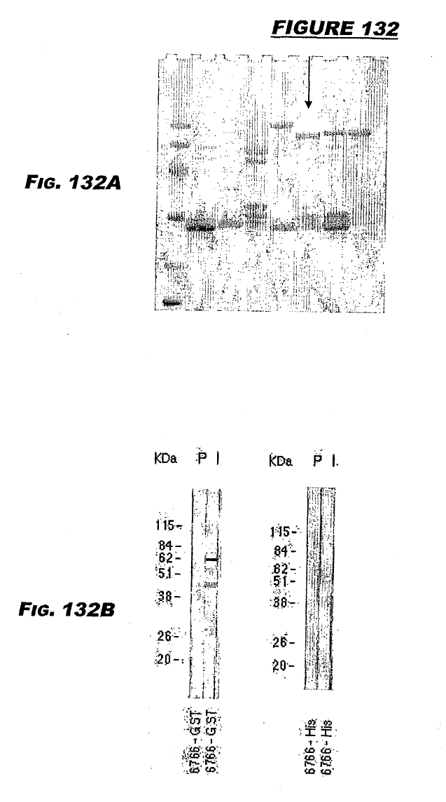

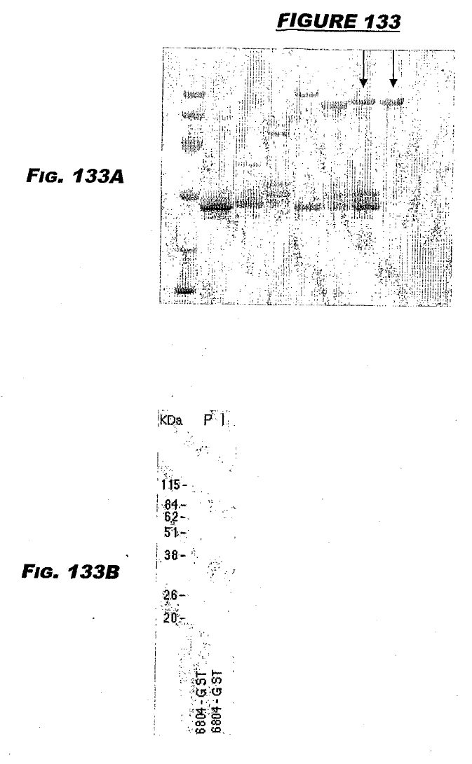

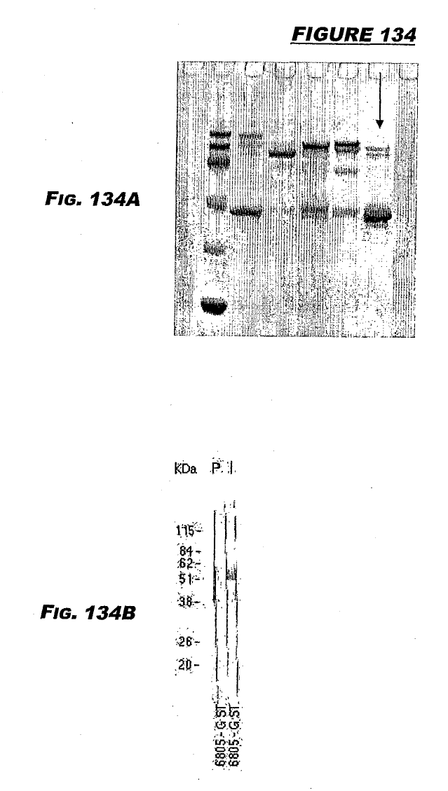

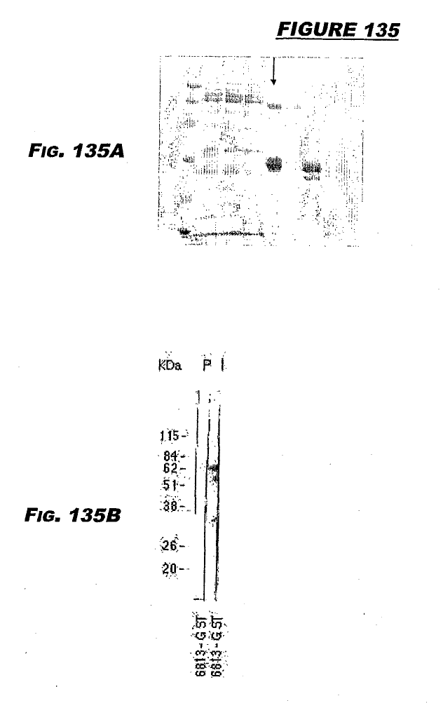

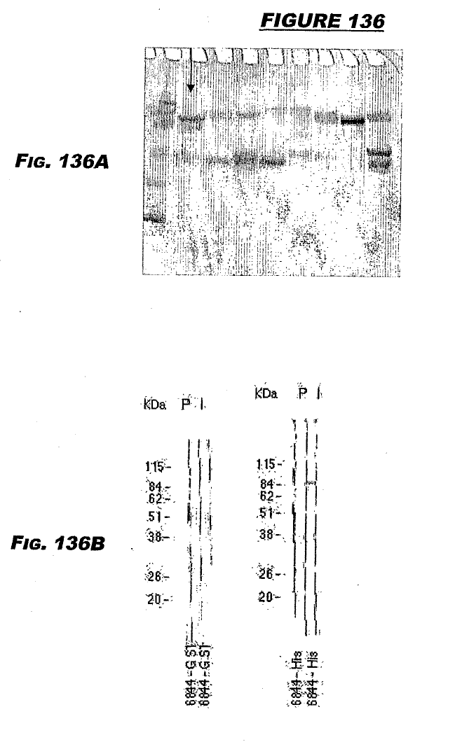

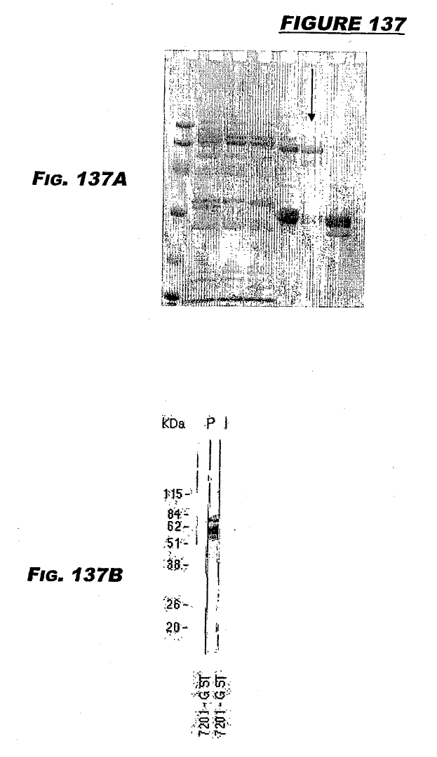

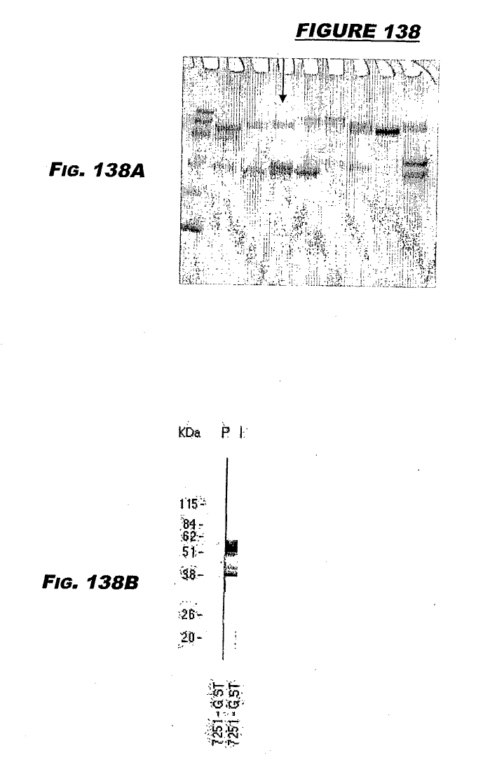

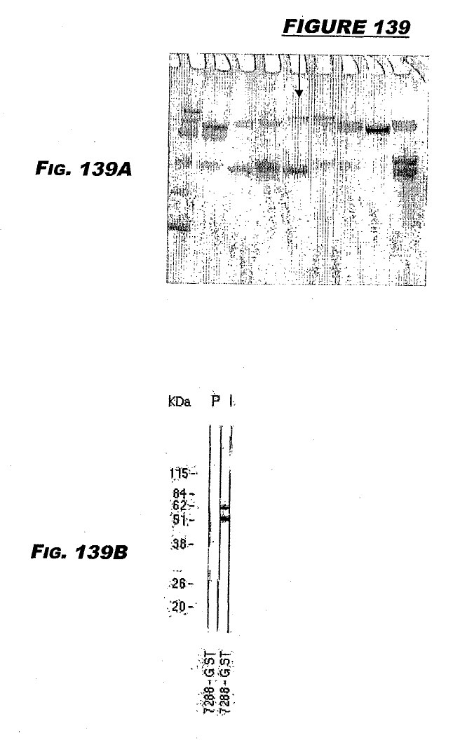

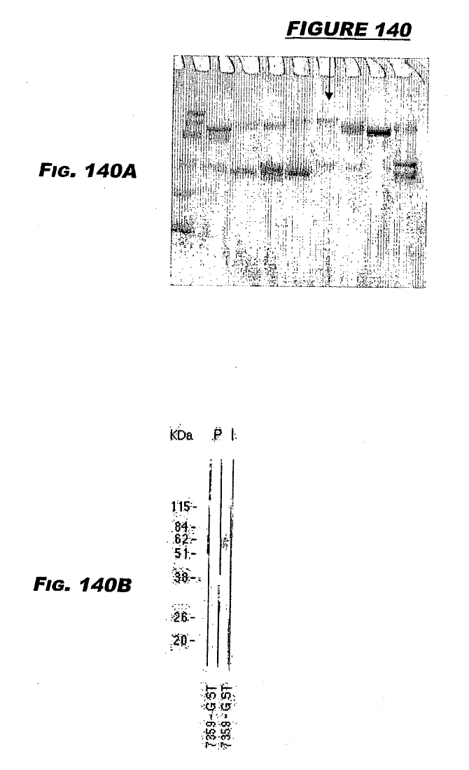

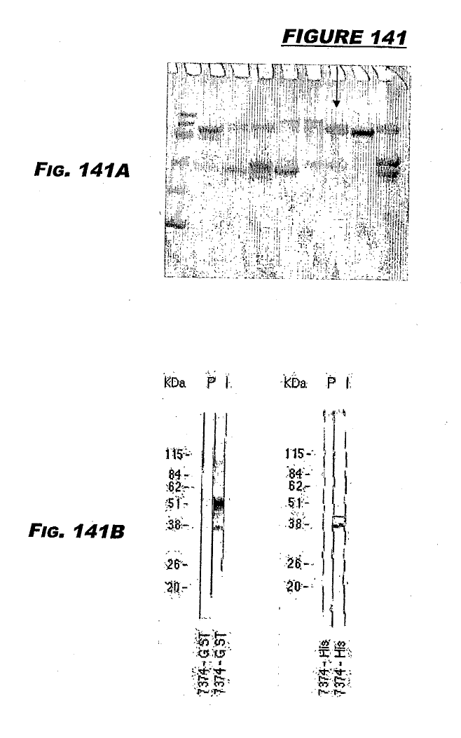

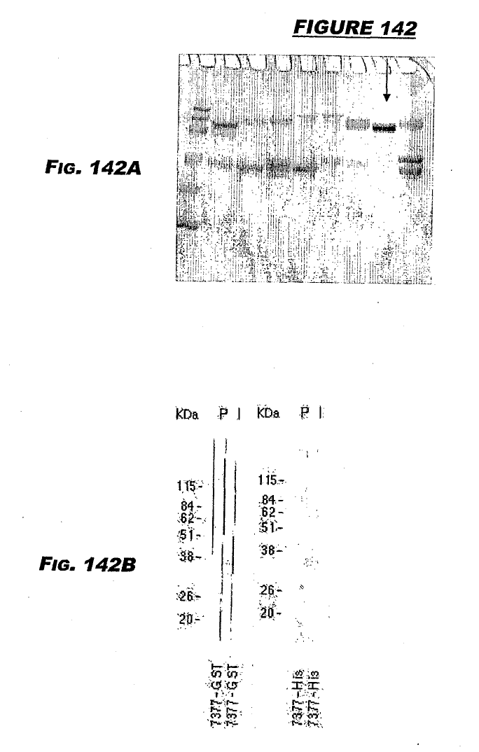

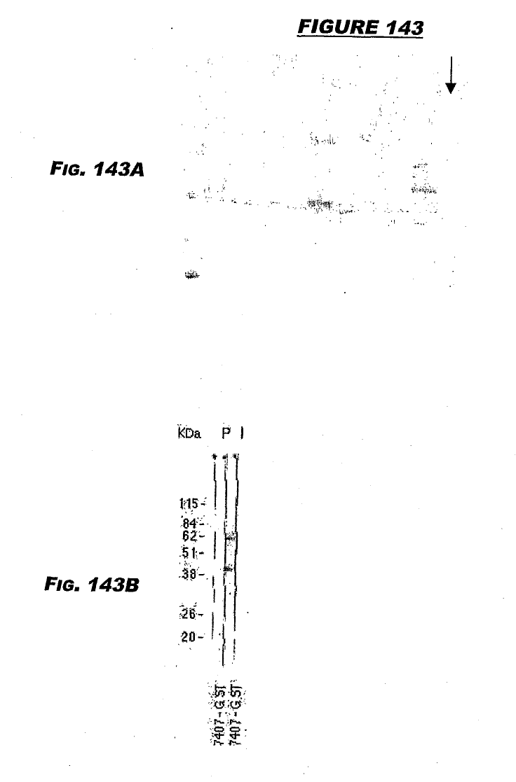

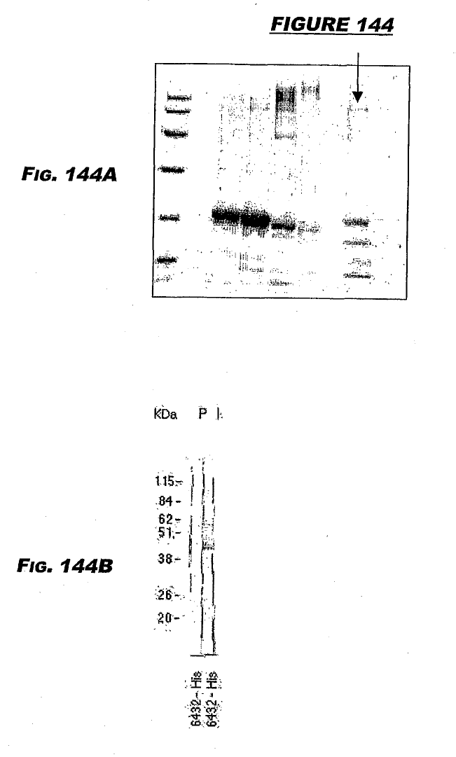

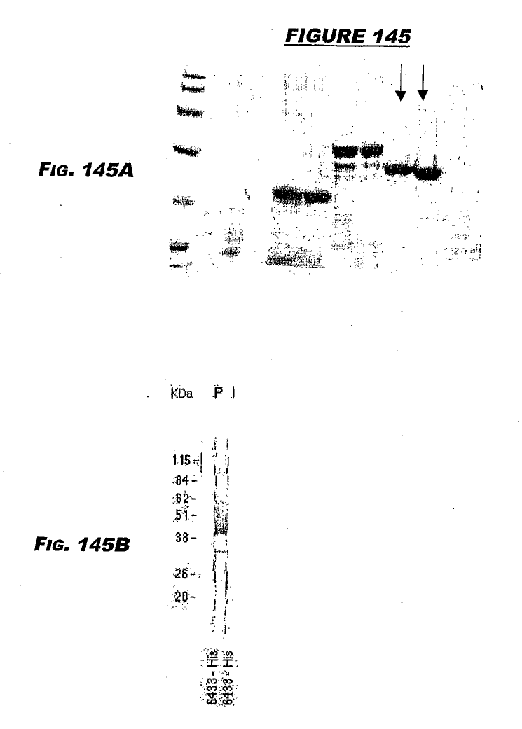

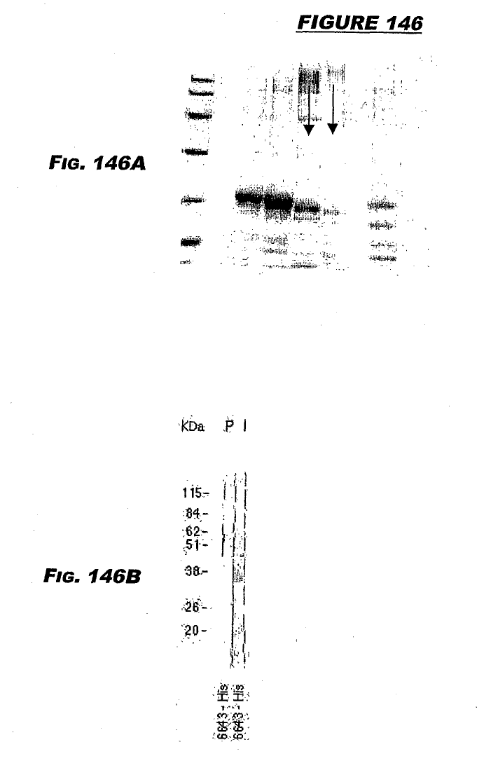

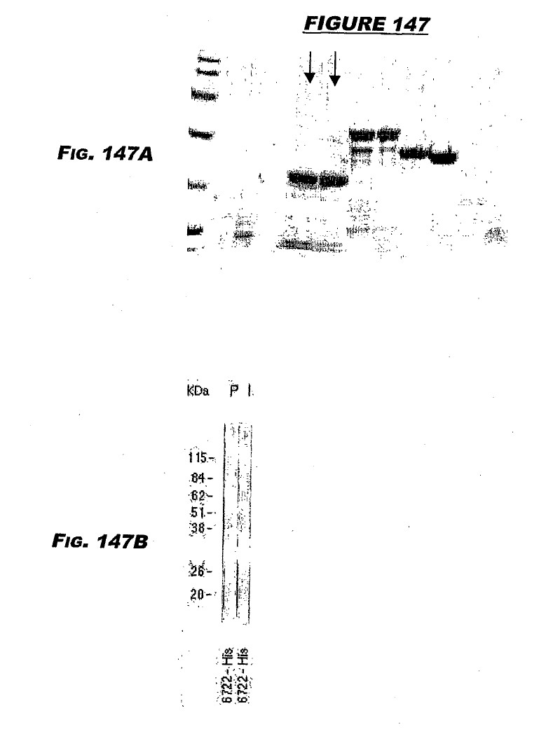

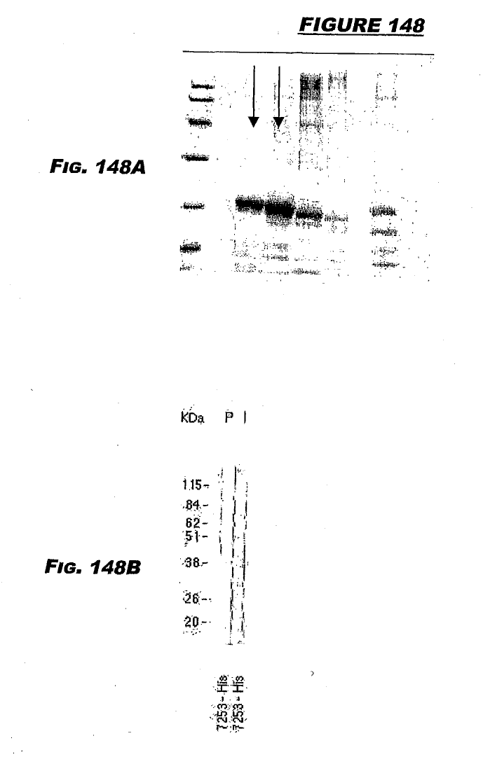

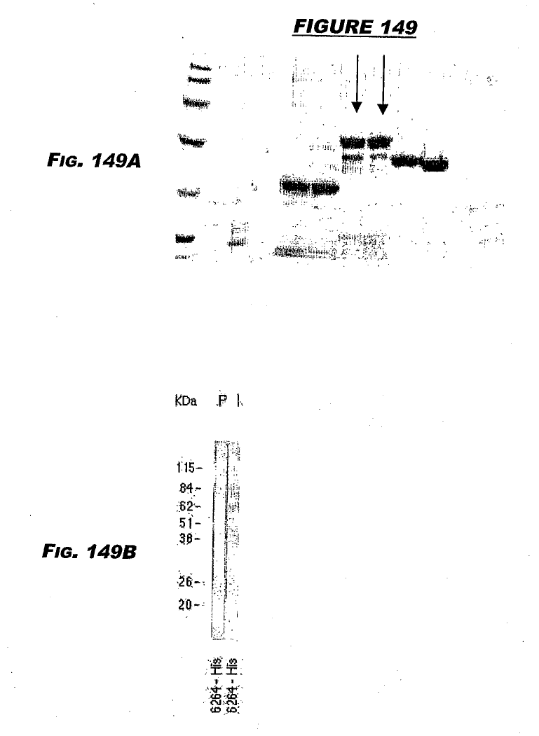

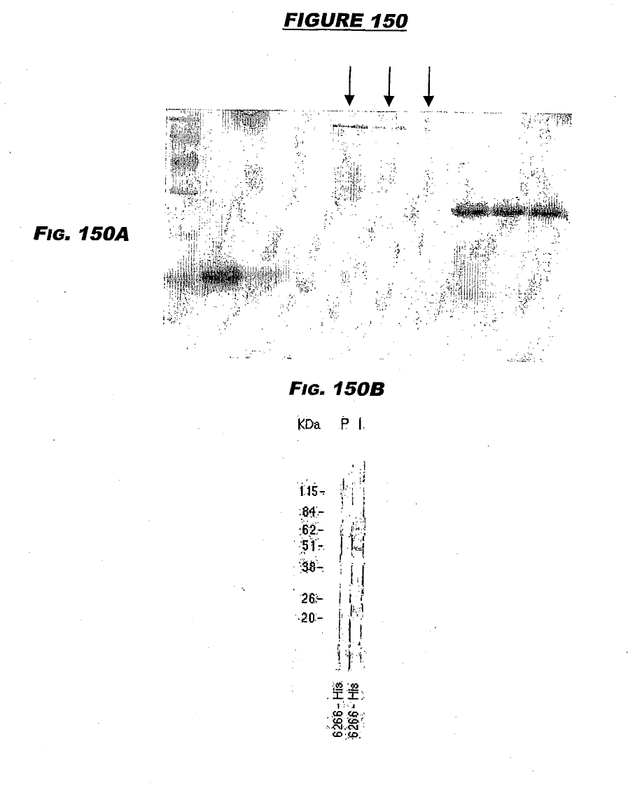

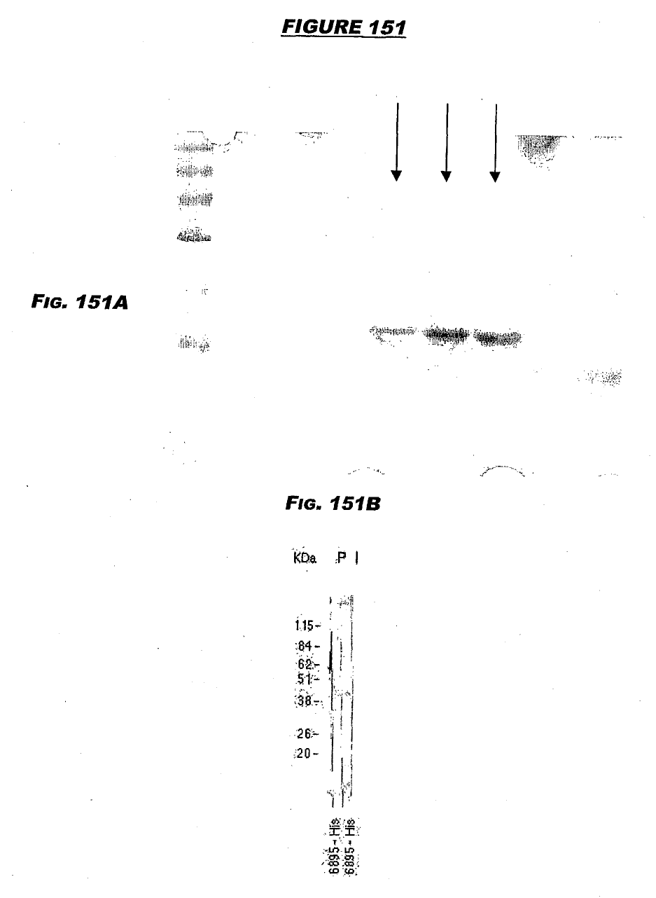

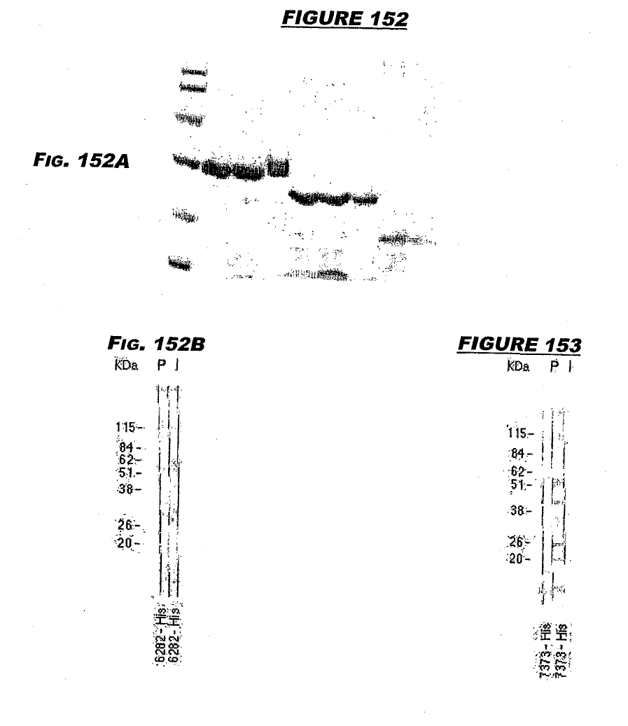

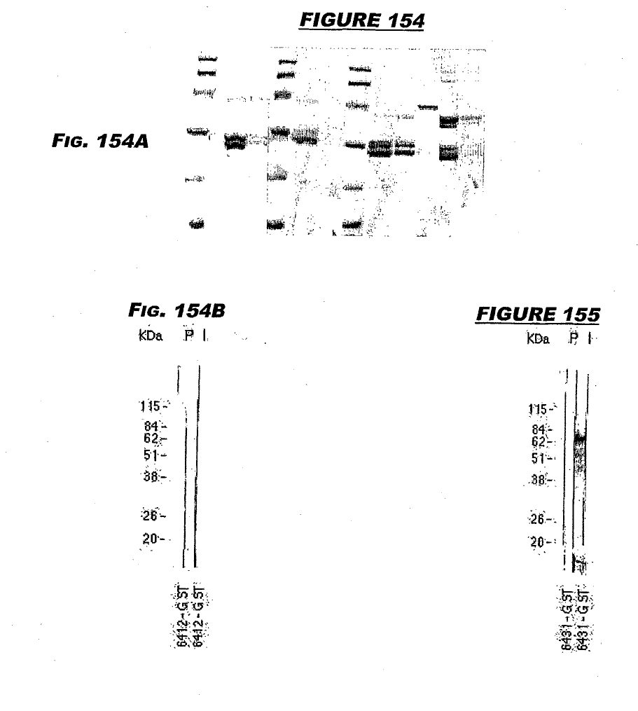

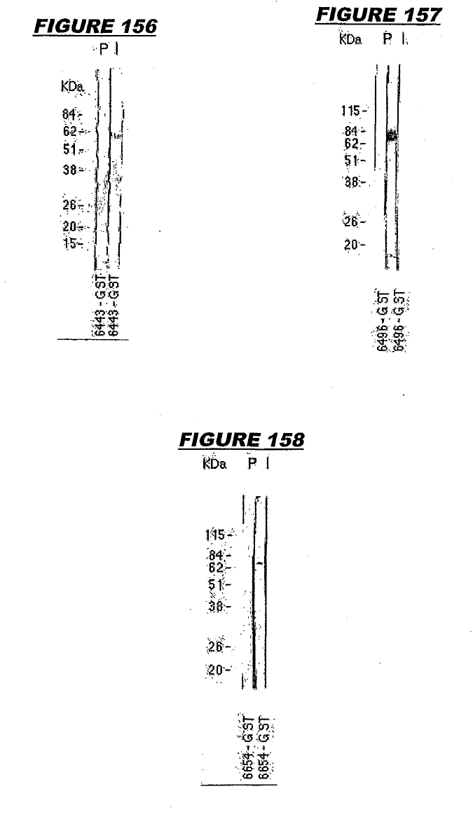

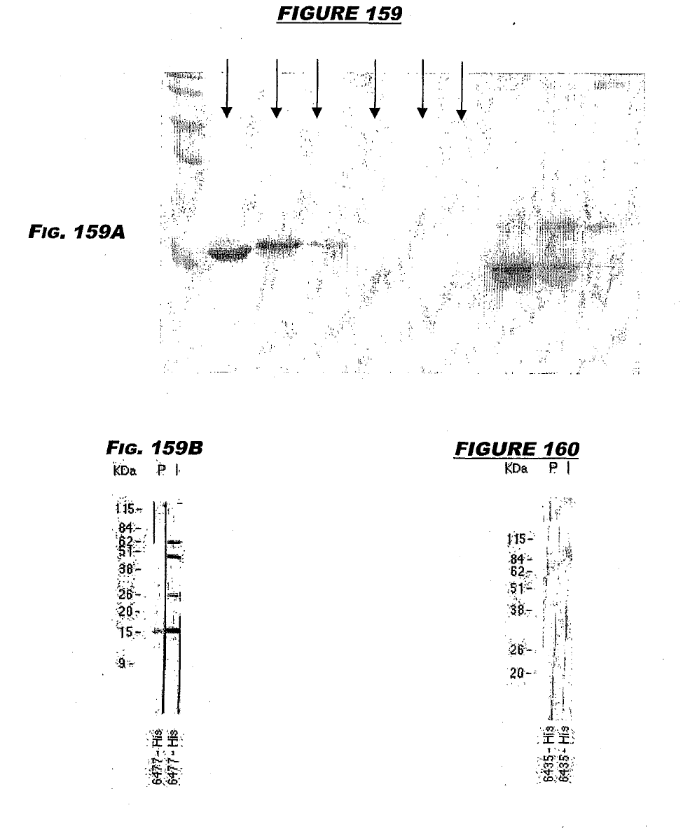

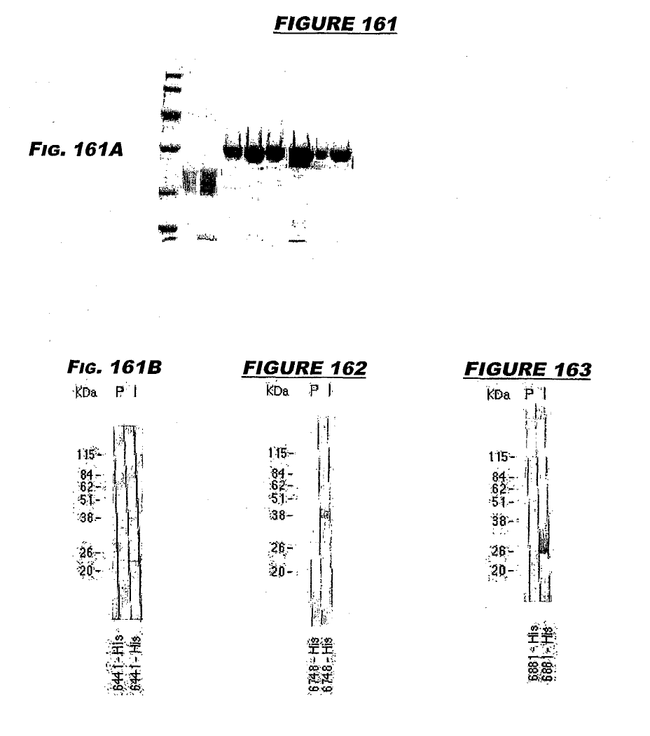

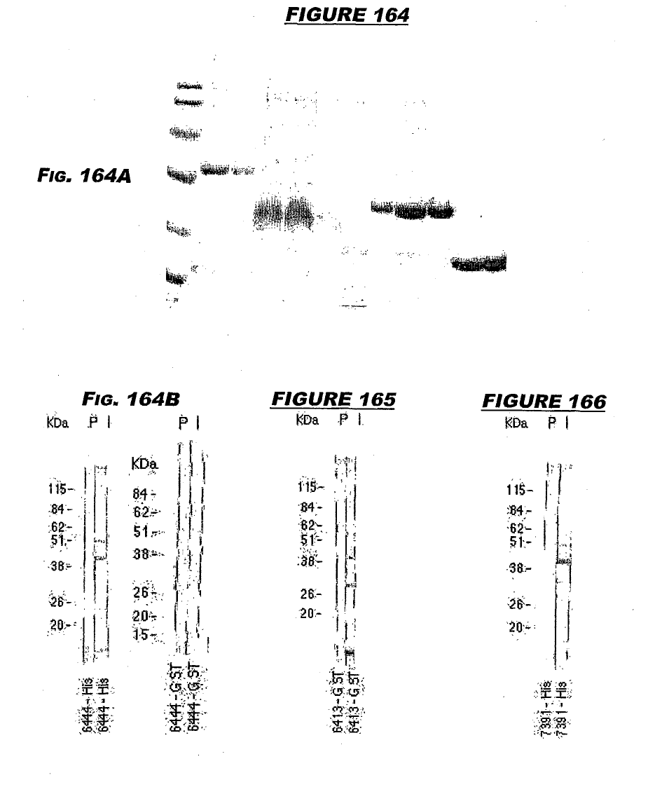

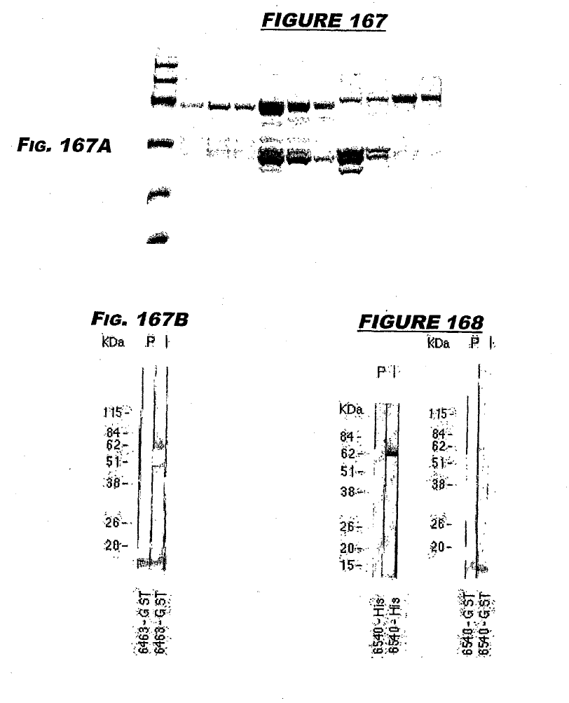

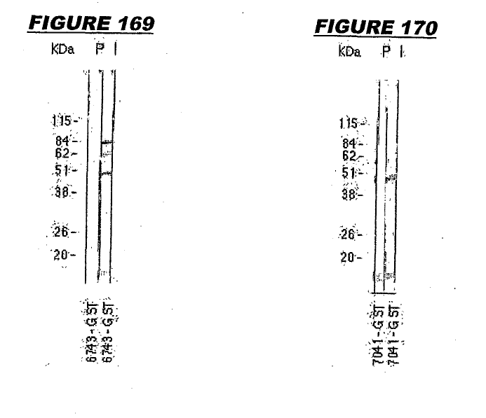

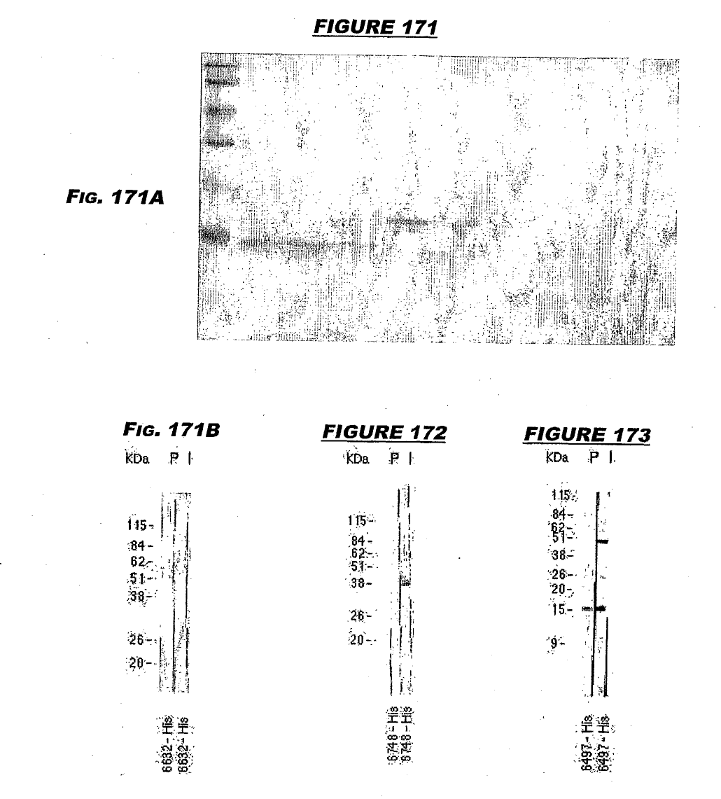

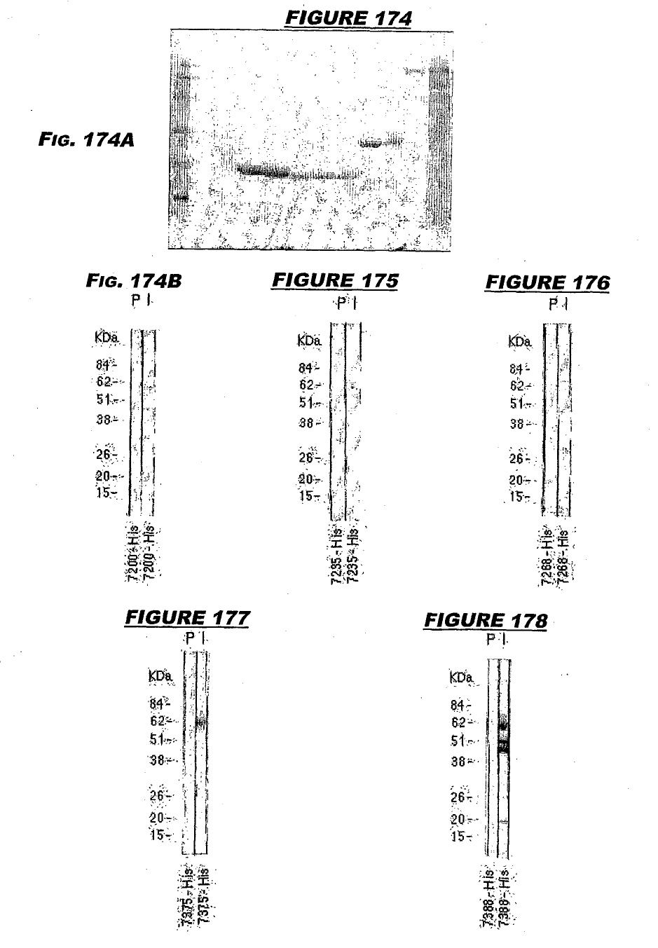

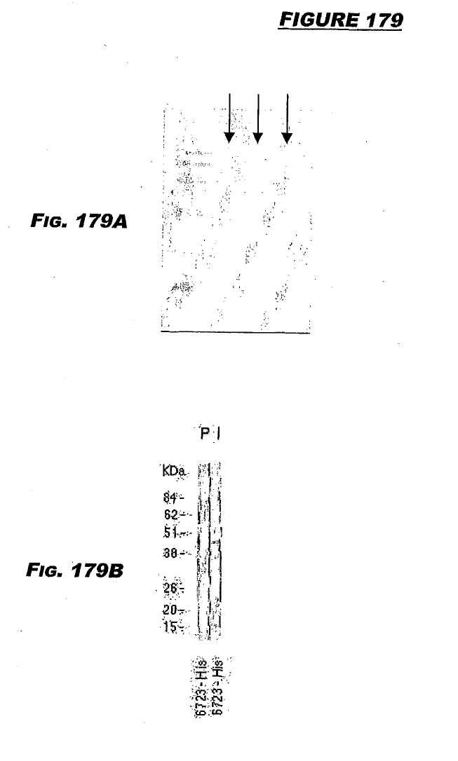

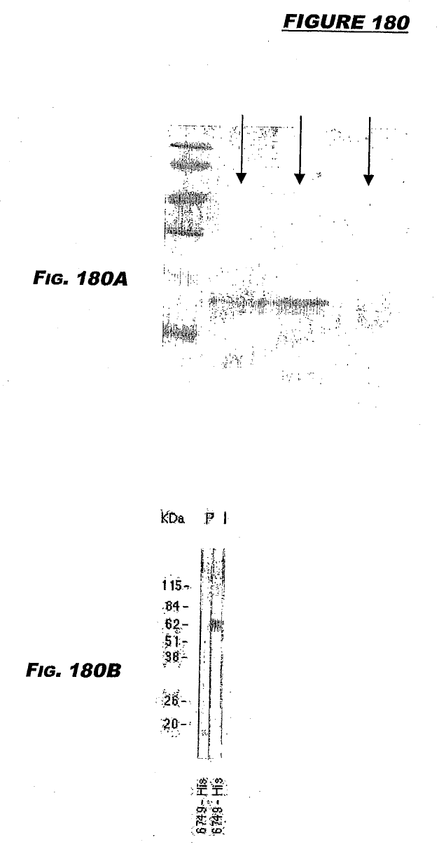

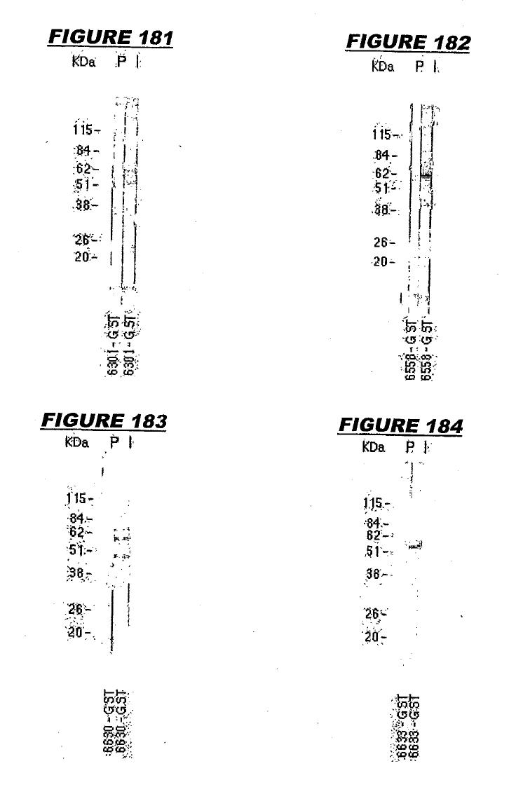

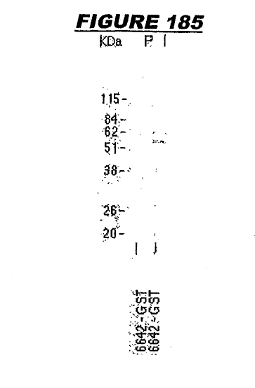

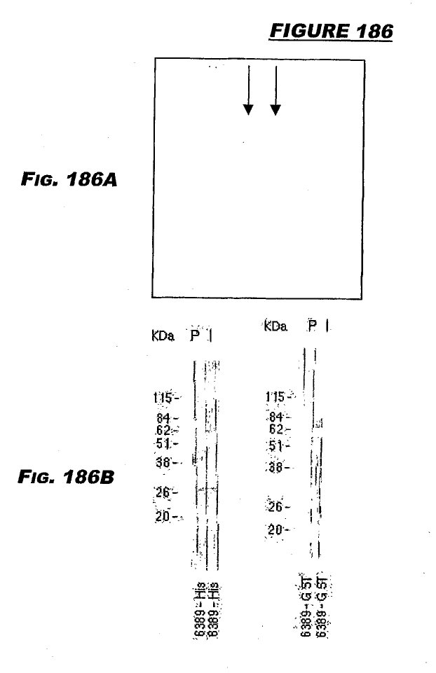

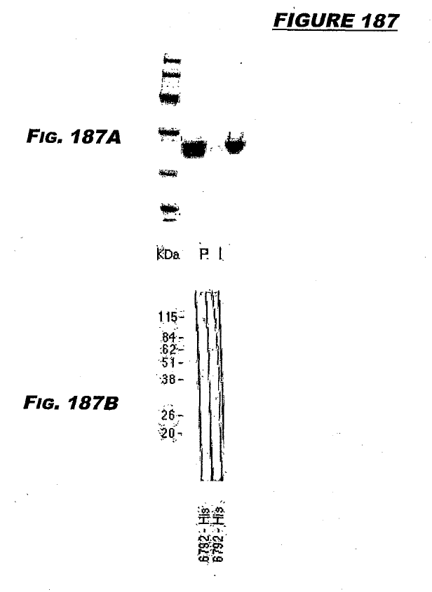

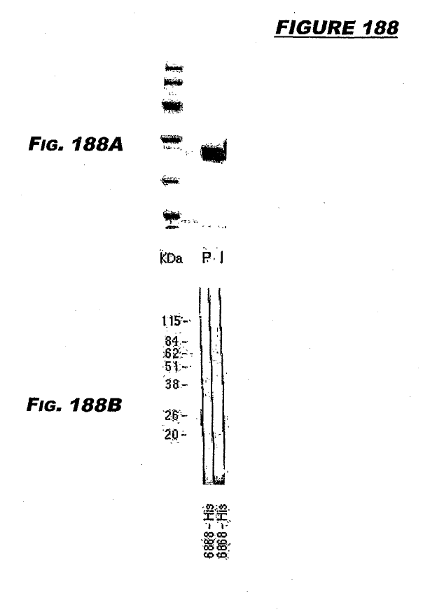

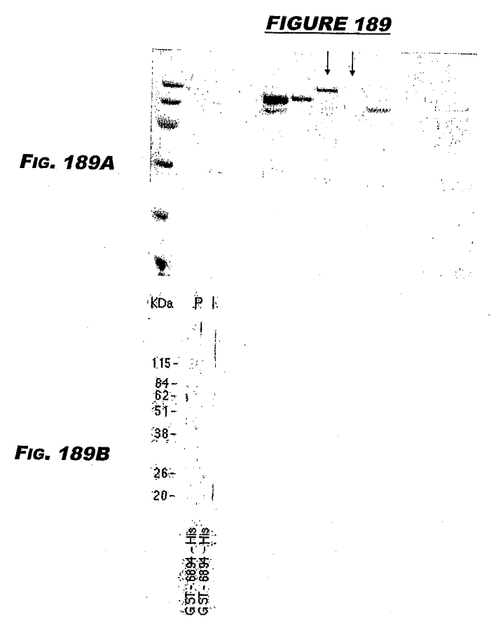

The protein was expressed in E. coli and purified as a his-tag product (FIG. 189A) and also in GST/his form. The recombinant proteins were used to immunise mice, whose sera were used in a Western blot (FIG. 189B) and for FACS analysis.

These experiments show that cp6894 is a surface-exposed and immunoaccessible protein, and that it is a useful immunogen. These properties are not evident from the sequence alone.

### Example 190

The following C. pneumoniae protein (PID 4377193) was identified in the 2D-PAGE experiment <SEQ ID 379; cp7193>:

| | |
|---|---|
| 1 | MKRVIYKTIF CGLTLLTSLS SCSLDPKGYN LETKNSRDLN QESVILKENR |
| 51 | ETPSLVKRLS RRSRRLFARR DQTQKDTLQV QANTKTYAEK ISEQDERDLS |
| 101 | FVVSSAAEKS SISLALSQGE IKDALYRIRE VHPLALIEAL AENPALIEGM |
| 151 | KKMQGRDWIW NLFLTWLSEV FSQAWSQGVI SREDIAAFAS TLGLDSGTVA |
| 201 | SIVQGERWPE LVDIVIT* |

A predicted leader peptide is underlined.

The cp7193 nucleotide sequence <SEQ ID 380> is:

| | |
|---|---|
| 1 | ATGAAAAGAG TCATTTATAA AACCATATTT TGCGGGTTAA CTTTACTTAC |
| 51 | AAGTTTGAGT AGTTGTTCCC TGGATCCTAA AGGATATAAC CTAGAGACAA |
| 101 | AAAACTCGAG GGACTTAAAT CAAGAGTCTG TTATACTGAA GGAAAACCGT |
| 151 | GAAACACCTT CTCTTGTTAA GAGACTCTCT CGTCGTTCTC GAAGACTCTT |
| 201 | CGCTCGACGT GATCAAACTC AGAAGGATAC GCTGCAAGTG CAAGCTAACT |
| 251 | TTAAGACCTA CGCAGAAAAG ATTTCAGAGA AGGACGAAAG AGACCTTTCT |
| 301 | TTCGTTGTCT CGTCTGCTGC AGAAAAGTCT TCAATTTCGT TAGCTTTGTC |
| 351 | TCAGGGTGAA ATTAAGGATG CTTTGTACCG TATCCGAGAA GTCCACCCTC |
| 401 | TAGCTTTAAT AGAAGCTCTT GCTGAAAACC CTGCCTTGAT AGAAGGGATG |
| 451 | AAAAAGATGC AAGGCCGTGA TTGGATTTGG AATCTTTTCT TAACACAATT |
| 501 | AAGTGAAGTA TTTTCTCAAG CTTGGTCTCA AGGGGTTATC TCTGAAGAAG |
| 551 | ATATCGCCGC ATTTGCCTCC ACCTTAGGTT TGGACTCCGG GACCGTTGCG |
| 601 | TCCATTGTCC AAGGGGAAAG GTGGCCCGAG CTTGTGGATA TAGTGATAAC |
| 651 | TTAA |

The PSORT algorithm predicts periplasmic (0.925).

This shows that cp7193 is an immunoaccessible protein in the EB and that it is a useful immunogen. These properties are not evident from the protein's sequence alone.

It will be appreciated that the invention has been described by way of example only and that modifications may be made whilst remaining within the spirit and scope of the invention.

TABLE II

Sequences of the Primers used to Amplify Cpn Genes.

| | | |
|---|---|---|
| Orf ID | N-terminus final primer | C-terminus final primer |

| | | |
|---|---|---|
| CP0014P | GCGTC CCG GGTCATATG AAGTCTTCTTTCCCCA | GCGT CTC GAG ATGAAAGAGTTTTTGCG |
| CP0015P | GCGTCCCGGGTCATATG TCAGCTCTGTTTTCTGA | GCGT CTC GAG GAATTGGTATTTTGCTC |
| CP0016P | GCGTCCCGGGTCATATG GCCGATCTCACATTAG | GCGT CTC GAG GTCCAAGTTAAGGTAGCA |
| CP0017P | GCGT CCG GGTCATATG GGTATCAAGGGAACTG | GCCGT CTC GAG AAATCCGAATCTTCC |
| CP0019P | GCGTCCCGGGTCAT ATGCAAGACTCTCAAGACTATAG | GOUT CTC GAG AAATCGGTATTTACCC |
| CP626OP | GCGTC CCG GGT GCTAGCACTACGATTTCTTTAACCC | GCGT CTC GAG AAAACGAAATTTGCTTC |
| CP6397P | GCGTC CCG GGTCATATGTTTAAACTGCTAAAAAATCTATT | GCGT CTC GAG ATGAAAGAAGAGTCCTCG |
| CP6456P | GCGTC CCG GGT CATATG TCATCTCCTGTAAATAACA | CGGT CTC GAG CTGACCATCTCCTGTT |
| CP6466P | GCGTC CCG GGT CAT ATG TGCAAGGAGTCCAGT | GCGT CTC GAG ATTTTCCTTAGCATAACG |
| CP6467P | GCGTC CCG GGT CAT ATG TGTTCCCCATCOOAA | GCGT CTC GAG TAGTTTTTCTATAAAACGAAAGTCT |
| CP6468P | GCGTC CCG GGT CAT ATG TGCTCCTCCTACTCTTC | GCGT CTC GAG GGGGAAATAGGTATATTTGA |
| CP6469P | GCGTC CCG GGT CAT ATG AGCTGCTCAAAGCAA | GCGT CTC GAG ACTTAAGATATCGATATTTTTGA |
| CP6552P | GCGTC CCG GGT CAT ATC TGCCATAAGGAAGATG | GCGT CTC GAG ACCATTGTCTTGAGTCAT |
| CP6567P | GCGTC CCG GGT CAT ATG ACCTCACCGATCCCC | GCGT CTC GAG AGAAGCCGGTAGAGGC |
| CP6576P | GCGTC CCG GGT CAT ATG ACTGAAAAAGTTAAAGAAGG | GCGT CTC GAG GAA CATGCCCCCTAA |
| CP6727P | GCGTC CCG GGT CATATCGTACATCCACTAATGGC | GCGT CTC GAG GAAAGAATAACGAGTTCC |
| CP6729P | GCGTC CCG GGT CAT ATGGCAGATGCTTCTTTATC | GCGT CTC GAG GAATGAGTATCTTAGCC |
| CP6731P | GCGTC CCG GGT CATATGGCTGTTGTTGAAATCAAT | GCGTC CAT GGC GGC CGC GAACTGGAACTTACCTCC |
| CP6736P | GCGTC CCG GGT GCT AGCGTAGAAGTTATCATGCCTT | GCGTC CAT GGC GGC CGC AAATCGTAATTTGCTTC |
| CP6737P | GCGT GGA TCC CAT ATG GAGACTAGACTCGGAGG | GCGT CTC GAG AAATGTGGATTTTAGTCC |
| CP6751P | GCGTC CCG GGT GCT AGC AATGAAGGTCTCCAACT | GCGT CTC GAG AAATCTCATTCTACTCGC |
| CP6752P | GCGTGA ATT CAT ATGTTCGGGATGACTCCT | GCGT CTC GAG GAATTTTAAGGTACTTCCTG |
| CPG753P | GCGTC CCG GGT GCT AGCACTCCCTACTCTCATAGAG | GCGT CTC GAG AAACTTAAAGGTCGTTC |
| CP6767P | GCGTC CCG GGT CAT ATG ATAAAACAAATAGGCCGT | GCGT CTC GAG TTCGTAAGCAACTTCAGA |
| CP6829P | GCGTC CCG GGT CAT ATG AAGCAGATGCGTCTTT | GCGTC CAT GGC GGC CGC GAATACAAACCGGATCC |
| CP6830P | GCGTC CCG GGT CAT ATG GATCCCGCGTCTGTT | GCGT CTC GAG TAAACTAGAAAAAGTCGTC |
| CP6832P | GCGTC CCG GGT CAT ATG CATAAAGTAATAGTTTTCATTT | GCGT CTC GAG TAAACTAGAAAAAGTCGT |
| CP6848P | GCGTC CCG GGT CAT ATG TCATCAAATCTACATCCC | GCGT CTC GAG AACCGGAGCTATTTTAC |
| CP6849P | GCGTC CCC GGT GCT AGC AGCGGGGGTATAGAG | GCGT CTC GAG ATACACGTGGGTATTTTC |
| CPG850P | GCGTC CCG GGT CAT ATG TGCCGCATTGTAGAT | GCGT CTC GAG CTGTTTGCATCTGCC |
| CP6854P | GCGTC CCG GGT GCT AGC TCAATAGCTATTGCAAG | GCGT CTC GAG TTATCGAAATGTCTTTG |
| CP6879P | GCGTC CCG GGT CAT ATG GCAACACCCCGTCAA | GCGTC CAT GGC GCG CCG TCCTTGAAATTGCTCTTCG |
| CP6894P | GCGTC CCG GGT CAT ATG TATAAAAGATGTGTCGTAGA | GCGT CTC GAG GGATGTACTTAAGCACG |
| CP6900P | GCGTC CCG GGT CAT ATG AAGATAAAATTTTCTTGGAAG | GCGT AAG CTT GGGAAGACGATACCG |
| CP6952P | GCGTC CCG GGT CAT ATG CTCTCGGATCAATATATAGG | GCGT CTC GAG TCGAATTTCTTTTTTAGC |
| CP7034P | GCGTC CCG GGT CAT ATG AAAAAACAGGTATATCAATG | GCGT AAG CTT AAACGCTGAAATTATACC |
| CP7090P | GCGTC CCG GGT CAT ATC TGTAGCCTTTCCCCT | GCGT CTC GAG GCGTGCATGAATCTTA |
| CP7091P | GCGTC CCG GGT CAT ATC GAAGAATTAGAAGTTGTTGT | GCGT CTC GAG TAGTGTTCTCTTTATCGGT |
| CP7170P | GCGTC CCG GGT CAT ATG CTAGGGGCTGGAAACC | GCGT AAG CTT AAACTGCAGACCTGACG |
| CP7228P | GCGTC CCG GGT CAT ATG ACTGCTGTTCTTATTCTTACA | GCGT CTC GAG ATCTGAAAGCGGAGG |
| CP7249P | GCGTC CCG GGT CAT ATG ATCCCATCCCCTACC | GCGT CTC GAG ATCAGGTTGCTGAGACTT |
| CP7250P | GCGTC CCG GGT CAT ATG AATCTCAAACAGGTCT | GCGT CTC GAG ATTTTTTCTAGAGAGACTCTC |
| CP0018P | GTGCGT CATATG GCAACCACTCCACTAA | ACTCGCTA GCGGCCGC TAATGAGGTCCCCAG |
| CP6270P | GTGCGT CATATG AATTTATTAGGAGCTGCT | ACTCGCTA GCGGCCGC AAATTTGATTTTGCTACC |
| CP6735P | GTCGGT CATATG GCAGCACAAGTTGTATAT | ACTCGCTA GCGGCCGC TGGCGTAGAAGTCATC |
| CP6998P | GTCGGT CATATG TTGCCTGTAGGGAAC | ACTCGCTA GCGGCCGC GAATCTGAACTGACCAGA |
| CP7933P | GTGCGT CATATG GTTAATCCTATTGGTCCA | ACTCGCTA GCGGCCGC TTGGAGATAACCAGAATATA |
| CP7287P | GTGCGT CATATG TTACACAGCTCAGAACTAGA | ACTCGCTA GCGGCCGC GAAAATAATACGGATACCA |
| CP0010P | GTGCGT CATATG GCAACTGCTGAAAATATA | GCGT CTCGAG GAATTGGAACTTACCC |
| CP0468P | GTGCGT GCTAGC ATTTTTTATGACAAACTCTAT | GCGT CTCGAG AAATGTGCAATGACTCT |
| CP6272P | GTGCGT CATATG TTGACTCATCAAGAGGCT | GCGT CTCGAG GAAGGGAGGTTTTTTAGGT |
| CP6273P | GTGCGT CATATG ACATATCTGGAAGCTC | ACTCGCTA GCGGCCGC CTCCACAATTTTTATG |
| CP6362P | GTGCGT CATATC CCCTTTGATATTACTTATTATACA | GCGT CTCGAG TCGTTTCCAAATCCA |
| CP6372P | GTGCGT CATATG AAACAACACTATTCTCTAAATA | GCGT CTCGAG TTTCTDGTGGTTTTTCT |
| CP6390P | GTGCGT CATATG CGAGAGGTGCCTAAG | ACTCGCTA GCGGCCGC TCTCCTAGACAGCCTT |
| CP6402P | GTGCGT CATATG AATGTTGCGGATCTCCTTT | GCGT CTCGAG GAAGGGGTTGGCCGT |
| CP6446P | GTGCGT CATATG TGTAATCAAAAGCCCTCTT | GCGT CTCGAG GGGCTGAGGAGGAAC |
| CP6520P | GTGCGT GCTAGC AAACACTACCTATCATTTTCT | GCGT CTCGAG CAGAAAGGCTTTTCTTT |
| CP6577P | GTGCGT CATATG AATTTAGGCTATGTTAATTTA | GCGT CTCGAG GTTTTGTTTTTTGAAAGA |
| CP6602P | GTGCGT CATATC GCAGCATCAGGAGGCA | GCGT CTCGAG TGACCAAGGATAGGGTTTAG |
| CP6807P | GTGCGT CATATG CCTCGTGGTGACACTTT | GCGT CTCCAG CGCTGCTTCTTGCTC |
| CP6615P | GTGCGT CATATG TGCTCTCAAAAAACGACAA | GCGT CTCGAG TGAAGAGGCGCCATC |
| CP6624P | GTGCGT CATATG GATGCGAAAATGGGA | GCGT CTCGAG TCTTTGACATTCAAGAGC |
| CP6672P | GTGCGT CATATG ATTCCTACCATGTTAATG | GCGT CTCGAG GTCATACAATTTCCTTATATA |
| CP6679P | GTGCGT CATATG TGCACTCACTTAGGCT | GCGT CTCGAG CGAGTAGTTAGCACAAAC |
| CP6717P | CTGCCT GCTACG AACACAATCGTAGCTTCA | ACTCGCTA GCGGCCGC GGCTGGCATATAGGT |
| CP6784P | GTGCGT GCTAGC AAATCAAGATGTTCTATTGATA | GCGT CTCGAG TCCAAAACAACCCTCT |
| CP6802P | GTGCGT CATATG TGCGTAAGTTATATTAATTCCTT | GCGT CTCGAG CAGTCGGGCTTGTTG |
| CP6847P | GTGCGT CATATG TCGGATCTTTTACGAG | GCGT CTCGAG TTTTCTACACTGTTGTAATAAA |
| CP6884P | GTGCGT CATATG AATCAGCTGCTTTCT | GCGT CTCGAG AGAGAAGGTAATTGTACC |
| CP6886P | GTGCGT CATATG TGTCTACTTATTATCTATCTCTAC | GCGT CTCGAG TTCAGAAAAATGGCT |
| CP6890P | GTGCGT CATATG TCCCCACGACCACAA | GCGT CTCGAG TCCTGCAGCATTTAGC |
| CP6960P | GTGCGT CATATG TGTGACGTACGGTCTA | ACTCGCTA GCGGCCGC TTCACCTTGATTTCCT |
| CP6968P | GTGCGT CATATG TGCGATGCAAAAC | ACTCGCTA GCGGCCGC GGAGTATGCTTAGATATT |
| CP6969P | GTGCGT CATATC TCCTGTGGTTACTCTATT | ACTCGCTA GCGGCCGC AAAAAGGTCATAGTATACCT |
| CP7005P | GTGCGT CATATC AAAACTGTGATATTGAACA | GCGT CTCGAG CTGAGCTTCTATTTCTATTAT |
| CP7072P | GTGCGT CATATG CCCATTTATGGGAAA | GCGT CTCAG GTTGAGCAAAGGTTTG |
| CP7101P | GTGCGT CATATG TATTCGTGTTACAGCAA | GCGT CTCGAG GAAAAATTCTTTAGGGAG |
| CP7102P | GTGCGT CATATG GCCGCTAAAGCAAAT | GCGT CTCGAG TGAAAATGAAAGGATGGT |
| CP7105P | GTGCGT GCTAGC AGTCTATATCAAAAATGGTG | GCGT CTCGAG ATCTTTCATTTGGTTATCT |
| CP7106P | GTGCGT CATATG AAAGATTTGGGGACTCT | GCGT CTCGAG GAATCCTAAGGCATACCTA |
| CP7107P | GTGCGT GCTAGC AGTATAGTCAGAAATTCTGCA | GCGT CTCGAG GAAGCTAAGATTATAGCTACTTT |
| CP7108P | GTGCGT GCTAGC GCGGCCCTTTCCA | ACTCGCTA GCGGCCGC TTTATGTTATATGGAACAGATAGG |
| CP7109P | GTGCGT CATATG GGACATTTTATTGATATTG | ACTCGCTA GCGGCCGC ATCATCAAGGTAGATAAAG |
| CP7110P | GTGCGT CATATG GGTTATTGCTATGTAATTACA | GCGT CTCGAG TTCTGATTGGACTCCA |
| CP7127P | GTGCGT CATATG GTGGCTTTAACGATAGC | ACTCGCTA GCGGCCG GCAGCCATCGTATTC |
| CP7130P | GTGCGT CATATG TTCAATATGCGAGG | GCGT CTCGAG CTTCTTATTTGAACTTTG |
| CP7140P | GTGCGT CATATG ACAGCCGGAGCAGCT | GCGT CTCGAG AGCACCCTCAATTTCATTG |
| CP7182P | GTGCGT CATATG GGATATGTTTTCTATGTGATC | GCGT CTCGAG GCTACTAAATCGAATCGA |
| CP6262P | GTGCGT CATATG ATCCCTGGATTAAGTTCA | ACTCGCTA GCGGCCGC TTCACTGGGAGCTTGA |
| CP6269P | GTGCGT CATATG TACCAGGAGAATCTAAGAT | ACTCGCTA GCGGCCGC GATTTTCTTCTTCAGCTC |
| CP6298P | GTGCGT CATATG GAGGAGGTGTCTGAGTAT | ACTCGCTA GCGGCCGC ATGTTTCTTTTTACTCTTTCT |
| CP6419P | GTGCGT CATATG GCTCCAGTCCGTGTT | GCGT CTCCAG AAGTGTTCGTTGGAAGT |
| CP6601P | GTGCGT CATATG AATAAGCTACTCAATTTCGT | GCGT CTCGAG GAAAATCTGAATTCTTCCT |
| CP6639P | GTGCGT CATATG TTAAATTCAAGCAATTCA | GCGT CTCGAG AGGAACTAAAACCTCATCT |
| CP6664P | GTGCGT GCTAGC GTTTTATTTCATGCTCAA | ACTCGCTA GCGGCCGC CTTAGAAAGACTATTTTCTAAGTA |
| CP6696P | GTGCGT CATATG TGCGTGATAATGGG | GCGT CTCGAG ATTCATCTTCGTAAAGAT |
| CP6757P | GTGCGT CATATG GCAGTTGGTGGCGT | ACTCGCTA GCGGCCGC CTGTCCCTCTGGAGC |
| CP6790P | GTGCGT GCTAGC AGTGAACACAAAAAATCA | ACTCGCTA GCGGCCGC CTTATCGTCGTTATCAATA |
| CP6814P | GTGCGT CATATG CATGACGCACTTCTAAG | GCGT CTCGAG TACAGCTGCGCGA |
| CP6834P | GTGCGT CATATG GTTATGGGAACCTATATCG | GCGT CTCGAG TACATTTGTATTGATTTCAG |
| CP6878P | GTGCGT CATATG AACGTCCCTGATTCC | GCGT CTCCAG GCTAGCGGCTCTTTC |
| CP6892P | GTGCGT CATATG CAGAAGCATCCTTCCT | ACTCGCTA GCGGCCGC TCCTCTTTAGGAAATGG |
| CP6909P | GTGCGT CATATG TCCTCTTTAGGAAATGG | GCGT CTCGAG CAGTGCCAAGTAGGGA |
| CP7015P | GTGCGT CATATG GCAGTACGATTAATTGTTG | GCGT CTCGAG TTTATTGTAGTCTATTTTATATTTC |
| CP7035P | GTGCGT GCTAGC AGCAGAAAAGACAATGA | GCGT CTCGAG ATTTTGAGTGTCTTGCA |
| CP7073P | GTGCGT CATATG ATTACCATAAATCACGTG | GCGT CTCGAG TATCCATCGACTTATAGC |
| CP7085P | CTGCGT GCTAGC TGTATTTTCCCTTACGTA | ACTCGCTA GCGGCCGC GGATTCTGCATACTCTG |
| CP7092P | GTCCGT CATATG TCTCCTCTTCCTAAAAAA | GCGT CTCGAG GGATTCATTACTGACCA |
| CP7093P | GTGCGT CATATG AAATACCGCTTCACG | GCGT CTCGAG ATTCTCTAGGGCTACGT |
| CP7094P | GTGCGT CATATG GTACACTTCTCTCATAACCC | GCGT CTCGAG TAAGTTTGTATTGCGGTAT |
| CP7132P | GTGCGT CATATG TTGTTATTAGGGACTTTAGGA | GCGT CTCGAG TTTCCCAACCGCA |
| CP7133P | GTGCGT CATATG GCTGCCAATGCTC | GCGT CTCGAG TAATTTAATACTCTTTGAAGG |
| CP7177P | GTGCGT CATATG CCTACTCAACTTAAAACAGA | GCGT CTCCAG AAGTTTATATTTCAGCACTT |
| CP7184P | GTGCCT GCTAGC CATATAGGATTTTCCCA | GCGT CTCGAG GTACTTACCAAAGCGAT |
| CP7206P | GTGCGT GCTAGC AAGAACCTATATCACCCTA | GCGT CTCGAG CACACCGAGCAAAC |
| CP7222P | GTGCGT CATATG GTAGTTTCAGAACAAAAAGTC | GCGT CTCGAG ACGTATGCGCAACTG |
| CP7223P | GTGCGT CATATG GAAGTATTAGACCGCTCT | CCGT CTCGAG CGAGAAAAAGCTTCC |
| CP7224P | GTGCGT CATATG ATGAAGAAAATTCGAAA | ACTCGCTA GCGGCCGC TAAGCATTCACAAATGA |
| CP7225P | GTGCGT CATATG CATAGTTTGCTTCATCGT | GCGT CTCGAG TCTTTTAACTAAATCTTGTTCTT |
| CP7303P | GTGCGT CATATG CTTGTCTATTGTTTTGATCC | GCGT CTCCAG AAAATATACGGAACTCGC |
| CP7304P | GTGCGT GCTAGC GAAGTTTATAGTTTTTCCC | GCGT CTCGAG TTTTTGATTCCTTAAGAAG |
| CP7305P | GTGCGT CATATG GAAGTTTATAGTTTTCACCCT | GCGT CTCGAG ACTCCTTGAGAAGGGAA |
| CP7307P | GTGCGT CATATG CTTAATCATGCTIGAAGC | ACTCCCTA GCCGCCGC CTCTTTTATTTTAGGAAGCT |
| CP7342P | GTGCGT CATATG AAAAAAAAATTTATTTTCTACT | ACTCGCTA GCGGCCGC CACACTCTGTTCTTCTG |
| CP7347P | GTGCGT CATATG TTTTCTAAGGATTTGACTAA | GCGT CTCGAG CGAAGCAGAAGTCGT |
| CP7353P | GTGCGT CATATG AATATGCCTGTTCCTTCT | GCGT CTCGAG GGGGCGTAGGTTGTA |
| CP7193P | GTGCGT CATATG TGTTCCCTGGATCCT | ACTCGCTA GCGGCCGC AGTTATCACTATATCCACAAG |
| CP7248P | GTGCCT GCTAGC CTTGAACATTCTAAACAACAT | GCGT CTCGAC ACGTAGTTTAAGAGCAGACT |
| CP7261P | GTGCGT CATATG TGTCTATCTGCCTACATAG | GCGT CTCGAG TTTTGATGCTTCTTTCA |
| CP7280P | CTGCGT CATATC GACCAGAAAATTGAAAA | GCGT CTCGAG AGAGGTCTTCTGAGTGC |
| CP7302P | GTGCGT CATATG AATTTCCATTGTAGTGTAGT | GCGT CTCGAG GAACAGTTCGATTTGTG |
| CP73O6P | GTGCGT CATATG CTTCCTTTATCAGGGCA | ACTCGCGA GCGCCCGC TTCTTCAGGTTTCAGG |
| CP7367P | GTGCGT GCTAGC CGTTATGCCGACGTC | GCGT CTCGAG TTCGTGCATTTGGTG |
| CP7408P | GTGCGT CATATG TTGAAAATCCACAAAAA | GCGT CTCGAG ATTTTTCGGAAGAG |
| CP7409P | GTGCGT CATATG AGACCTTATCTTGTCATGGT | GCGT CTCGAG CCCTTTGCTCTTTACATAG |
| CP6733P | GTGCGT ACTAGT TGTCACCTACAGTCACTAG | GCGT CTCGAG GAATCGGAGTTTGGTA |
| CP6728P | GTGCGT ACTAGT AAGTCCTCTGTCTCTTGG | GCGT CTCGAG GAAACAAAACTTAGAGCCC |

TABLE III
Proteins with best results in FACS analysis

| | Molecular Weight (kDa) | | |
|---|---|---|---|
| cp number | Theoretical | Western Blot | Fusion type |
| 6260 | 97.5 | 94; 70 | GST |
| 6270 | 87.5 | — | GST |
| 6272 | 78.0 | 90 | GST |
| 6273 | 58.6 | 74; 64; 50 | GST |
| 6296 | 31.1 | — | GST |
| 6390 | 88.9 | 102 | GST |
| 6456 | 42.5 | 89; 6745 | GST |
| 6466 | 57.5 | 59; 56 | His |
| 6467 | 59.0 | 67 | GST |
| 6552 | 28.4 | 50; 27 | GST |
| 6576 | 86.0 | 79; 70; 62; 45 | GST |
| 6577 | 17.3 | 12 | GST |
| 6602 | 43.4 | 53; 42; 34 | GST |
| 6664 | 54.5 | 104; 45 | GST |
| 6696 | 47.9 | 95; 53 | GST |
| 6727 | 130.0-142.9 | 123; 61; 39 | His |
| 6729 | 94.8 | multiple bands | GST |
| 6731 | 95.5 | 97 | HST |
| 6733 | 97.1 | 104 | His |
| 6736 | 100.1 | 98; 93; 66; 60 | GST |
| 6737 | 101.2 | multiple bands | GST |
| 6751 | 100.2 | 95;71 | GST |
| 6752 | 102.1 | 97;48 | His |
| 6767 | 29.1 | 28 | GST |
| 6784 | 32.9 | 35 | GST |
| 6790 | 71.3 | multiple bands | His |
| 6802 | 29.7 | — | GST |
| 6814 | 29.6 | 28 | GST |
| 6830 | 177.4 | 174; 91; 13 | GST |
| 6849 | 57.3 | multiple bands | GST |
| 6850 | 7.4-9.4 | 61; 14; 8 | GST |
| 6854 | 42.2 | — | GST |
| 6878 | 40.4 | — | GST |
| 6900 | 28.0 | — | GST |
| 6960 | 25.6 | 75; 35 | GST |
| 6968 | 34.6 | 83; 53; 35 | GST |
| 6998 | 39.3 | multiple bands | GST |
| 7033 | 68.2 | multiple bands | GST |
| 7101 | 113 | 105 | GST |
| 7102 | 63.4 | — | GST |
| 7105 | 29.2 | 30 | GST |
| 7106 | 39.5 | 72;46 | GST |
| 7107 | 71.4 | 67;31 | His |
| 7108 | 35.9 | 35 | GST |
| 7111 | 46.1 | 51 | GST |
| 7132 | 17.9 | 57; 47; 17 | His |
| 7140 | 36.2-29.8 | 50; 38; 34 | GST |
| 7170 | 34.4 | 77;33 | GST |
| 7224 | 39.4 | 40 | GST |
| 7287 | 167.3 | 180 | GST |
| 7306 | 50.1 | 50 | GST |

TABLE V
FACS-positive proteins not found in C.trachomatis

| | |
|---|---|
| cp7105 | cp6390 |
| cp7106 | cp6784 |
| cp7107 | cp6296 |
| cp7108 | |

TABLE V
Proteins identified by MALDI-TOF following 2D electrophoresis

| | | |
|---|---|---|
| cp6270 | cp6733 | cp6900 |
| cp6552 | cp6736 | cp6960 |
| cp6576 | cp6737 | cp6998 |
| cp6577 | cp6752 | cp7033 |
| cp6602 | cp6767 | cp7108 |
| cp6664 | cp6784 | cp7111 |
| cp6727 | cp6790 | cp7170 |
| cp6728 | cp6830 | cp7287 |
| cp6729 | cp6849 | cp7306 |

1. A protein comprising an amino acid sequence selected from the group consisting of SEQ IDs 97, 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, & 377.

2. A protein having 50% or greater sequence identity to a protein according to claim 1.

3. A protein comprising a fragment of an amino acid sequence selected from the group consisting of SEQ IDs 97, 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, & 377.

4. A nucleic acid molecule which encodes a protein according to any one of claims 1 to 3.

5. A nucleic acid molecule according to claim 4, comprising a nucleotide sequence selected from the group consisting of SEQ IDs 98, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, & 378.

6. A nucleic acid molecule comprising a fragment of a nucleotide sequence selected from the group consisting of SEQ IDs 98, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, & 378.

7. A nucleic acid molecule comprising a nucleotide sequence complementary to a nucleic acid molecule according to any one of claims 4 to 6.

8. A nucleic acid molecule comprising a nucleotide sequences having 50% or greater sequence identity to a nucleic acid molecule according to any one of claims 4 to 7.

9. A nucleic acid molecule which can hybridise to a nucleic acid molecule according to any one of claims 4 to 8 under high stringency conditions.

10. A composition comprising a protein or a nucleic acid molecule according to any preceding claim.

11. A composition according to claim 10 being a vaccine composition.

12. A composition according to claim 10 or claim 11 for use as a pharmaceutical.

13. The use of a composition according to claim 10 in the manufacture of a medicament for the treatment or prevention of infection due to Chlamydia bacteria, particularly Chlamydia pneumoniae.
